(19) United States
(12) Patent Application Publication
(10) Pub. No.: US 2005/0113450 A1
(43) Pub. Date: May 26, 2005

(54) Antibacterial agents

(76) Inventors: Atli Thorarensen, O'Fallon, MI (US); J. Craig Ruble, Greenwood, IN (US); Donna Lee Romero, Chesterfield, MI (US)

Correspondence Address: PHARMACIA & UPJOHN, 301 HENRIETTA ST, 0228-32-LAW, KALAMAZOO, MI, 49007, US

(21) Appl. No.: 10/645,732

(22) Filed: Aug. 20, 2003

Related U.S. Application Data
(60) Provisional application No. 60/405,464, filed on Aug. 23, 2002.

(51) Int. Cl. A61K 31/195

(52) U.S. Cl. 514/562; 514/563; 514/567; 514/565; 562/426; 562/450; 562/439

(57) ABSTRACT

The present invention relates to antibacterial agents that are useful for sterilization, sanitation, antisepsis, and disinfection

### CROSS REFERENCE

This application claims the benefit of U.S. Ser. No. 60/405,464, filed on Aug. 23, 2002, under 35 U.S.C. § 119(e)(i).

### FIELD OF THE INVENTION

The present invention relates to antibacterial agents that are useful for sterilization, sanitation, antisepsis, and disinfection.

### BACKGROUND

The inappropriate growth of a variety of bacteria has been a problem for many years. Bacteria have caused degradation of natural product materials, infection in humans and other animals, and spoilage of foods.

Sterilization denotes the use of either physical or chemical agents to eliminate all viable bacteria from a material, while disinfection generally refers-to the use of germicidal chemical agents to destroy the potential infectivity of a material. Sanitizing refers to procedures used to simply lower the bacterial content of utensils used for food. Antisepsis refers to the topical application of chemicals to a body surface to kill or inhibit pathogenic microbes. Disinfectants are widely used for skin antisepsis in preparation for surgery.

Bacteria are the smallest organisms that contain all the machinery required for growth and self-replication. A bacterium includes a rigid cell wall surrounding the cytoplasmic membrane, which itself encloses a single naked chromosome without a nuclear membrane. The cytoplasmic membrane consists primarily of a bi-layer of lipid molecules.

The fundamental criterion of bactericidal action is loss of the ability of the organism to propagate indefinitely, when placed in a suitable environment. Bactericidal action suggests microbe damage of various types, including the triggering of irreversible damage to the cytoplasmic cell membrane or irreversible impairment of the DNA (or viral RNA replication. Accordingly, sterilization is not identical with destruction of microbes. Additionally, it is understood that damage to nucleic acids (DNA or RNA) is not always irreversible, as it is known that ultraviolet light-induced damage to viral nucleic acids can be repaired by enzymatic and genetic mechanisms.

### SUMMARY OF THE INVENTION

The invention relates to antibacterial agents that are useful for sterilization, sanitation, antisepsis, and disinfection.

In one aspect, the invention features methods of using antibacterial agents of formula I for sterilizing, sanitizing, antisepsis, or disinfecting. The method includes applying the antibacterial agent to a location in need of sterilization, sanitation, antisepsis, and disinfection. Specifically, a method of sterilization, sanitation, antisepsis, and disinfection, includes applying antimicrobial compounds to a surface in need of sterilization, sanitation, antisepsis, and disinfection. The antimicrobial compounds are applied in a therapeutically acceptable amount, e.g., an amount sufficient to kill or hinder the growth of bacteria on the surface to be sterilized, sanitized, or disinfected.

In general, the antibacterial agents have the formula 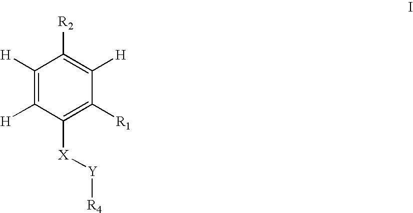
or a pharmaceutically acceptable salt thereof,
wherein
X═NH
Y═CO, CS, —C(═N—CN) or
X and Y together form an alkene, or C₃-C₅ cycloalkyl;
R₁ is —COOH;
R₂ is an electron withdrawing group;
R₄ is an optionally substituted aryl, provided that the aryl is not simultaneously substituted with a sulfonamide and a urea or thiourea, further provided that the aryl is not solely substituted at the ortho-position relative to Y, and still further provided that the aryl is not substituted with a group selected from 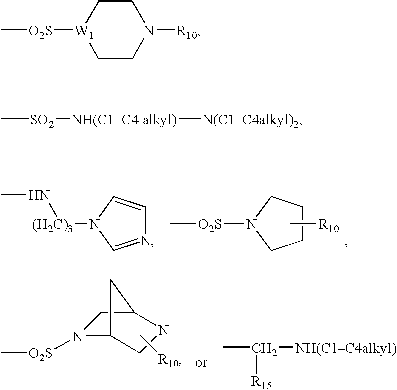
W₁ is N or CH;
R₁₀ is C₁-C₄ alkyl, C₁-C₄ substituted alkyl, Het, substituted Het, aryl, or substituted aryl; and
R₁₅ is H, C₁-C₄ alkyl, C₁-C₄ substituted alkyl, Het, substituted Het, C₄-C₇ cycloalkyl.

### DETAILED DESCRIPTION OF THE INVENTION

The term “halo” refers to a halogen atom selected from Cl, Br, I, and F.

The term “alkyl” refers to both straight- and branched-chain moieties. Unless otherwise specifically stated alkyl moieties include between 1 and 9 carbon atoms.

The term “alkenyl” refers to both straight- and branched-chain moieties containing at least one —C═C—. Unless otherwise specifically stated alkenyl moieties include between 1 and 9 carbon atoms.

The term “alkynyl” refers to both straight- and branched-chain moieties containing at least one —C≡C—. Unless otherwise specifically stated alkynyl moieties include between 1 and 9 carbon atoms. between 1 and 6 carbon atoms

The term “alkoxy” refers to —O-alkyl groups.

The term “cycloalkyl” refers to a cyclic alkyl moiety. Unless otherwise specifically stated cycloalkyl moieties will include between 3 and 9 carbon atoms.

The term “cycloalkenyl” refers to a cyclic alkenyl moiety. Unless otherwise specifically stated cycloalkenyl moieties will include between 5 and 9 carbon atoms and at least one —C═C— group within the cyclic ring.

The term “amino” refers to —NH₂.

The term “sulfonamide” refers to a —S(O)₂—N(Q₁₀)₂

The term “aryl” refers to phenyl and naphthyl.

The term “het” refers to mono- or bi-cyclic ring systems containing at least one heteroatom selected from O, S, and N. Each mono-cyclic ring may be aromatic, saturated, or partially unsaturated. A bi-cyclic ring system may include a mono-cyclic ring containing at least one heteroatom fused with an cycloalkyl or aryl group. A bi-cyclic ring system may also include a mono-cyclic ring containing at least one heteroatom filsed with another het, mono-cyclic ring system.

Examples of “het” include, but are not limited to, pyridine, thiophene, furan, pyrazoline, pyrimidine, 2-pyridyl, 3-pyridyl, 4-pyridyl, 2-pyrimidinyl, 4-pyrimidinyl, 5-pyrimidinyl, 3-pyridazinyl, 4-pyridazinyl, 3-pyrazinyl, 4-oxo-2-imidazolyl, 2-imidazolyl, 4-imidazolyl, 3-isoxazolyl, 4-isoxazolyl, 5-isoxazolyl, 3-pyrazolyl, 4-pyrazolyl, 5-pyrazolyl, 2-oxazolyl, 4-oxazolyl, 4-oxo-2-oxazolyl, 5-oxazolyl, 1,2,3-oxathiazole, 1,2,3-oxadiazole, 1,2,4-oxadiazole, 1,2,5-oxadiazole, 1,3,4-oxadiazole, 2-thiazolyl, 4-thiazolyl, 5-thiazolyl, 3-isothiazole, 4-isothiazole, 5-isothiazole, 2-furanyl, 3-furanyl, 2-thienyl, 3-thienyl, 2-pyrrolyl, 3-pyrrolyl, 3-isopyrrolyl, 4-isopyrrolyl, 5-isopyrrolyl, 1,2,3,-oxathiazole-1-oxide, 1,2,4-oxadiazol-3-yl, 1,2,4-oxadiazol-5-yl, 5-oxo-1,2,4-oxadiazol-3-yl, 1,2,4-thiadiazol-3-yl, 1,2,4-thiadiazol-5-yl, 3-oxo-1,2,4-thiadiazol-5-yl, 1,3,4-thiadiazol-5-yl, 2-oxo-1,3,4-thiadiazol-5-yl, 1,2,4-triazol-3-yl, 1,2,4-triazol-5-yl, 1,2,3,4-tetrazol-5-yl, 5-oxazolyl, 3-isothiazolyl, 4-isothiazolyl, 5-isothiazolyl, 1,3,4,-oxadiazole, 4-oxo-2-thiazolinyl, 5-methyl-1,3,4-thiadiazol-2-yl, thiazoledione, 1,2,3,4-thiatriazole, 1,2,4-dithiazolone, phthalimide, quinolinyl, morpholinyl, benzoxazoyl, diazinyl, triazinyl, quinolinyl, quinoxalinyl, naphthyridinyl, azetidinyl, pyrrolidinyl, hydantoinyl, oxathiolanyl, dioxolanyl, imidazolidinyl, and azabicyclo[2.2.1]heptyl.

The term “heteroaryl” refers to a mono- or bicylic het in which at least one cyclic ring is aromatic.

The term “substituted alkyl” refers to an alkyl moiety including 1-4 substituents selected from halo, het, cycloalkyl, cycloalkenyl, aryl, —OQ₁₀, —SQ₁₀, —S(O)₂Q₁₀, —S(O)Q₁₀, —OS(O)₂Q₁₀, —C(═NQ₁₀)Q₁₀, —C(═N—O-Q₁₀)Q₁₀, —S(O)₂—N═S(O)(Q₁₀)₂, —S(O)₂—N═S(Q₁₀)₂, —NQ₁₀Q₁₀, —C(O)Q₁₀, —C(S)Q₁₀, —C(O)OQ₁₀, —OC(O)Q₁₀, —C(S)NQ₁₀Q₁₀, —N(Q₁₀)C(S)NQ₁₀Q₁₀, —C(O)NQ₁₀Q₁₀, —C(O)C(Q₁₆)₂OC(O)Q₁₀, —CN, ═O, ═S, —NQ₁₀C(O)Q₁₀, —NQ₁₀C(O)NQ₁₀Q₁₀, —S(O)₂NQ₁₀Q₁₀, —NQ₁₀S(O)₂Q₁₀, —NQ₁₀S(O)Q₁₀, —NQ₁₀SQ₁₀, —NO₂, and —SNQ₁₀Q₁₀. Each of the het, cycloalkyl, cycloalkenyl, and aryl being optionally substituted with 1-4 substituents independently selected from halo and Q₁₅.

The term “substituted aryl” refers to an aryl moiety having 1-3 substituents selected from —OQ₁₀, —SQ₁₀, —S(O)₂Q₁₀, —S(O)Q₁₀, —OS(O)₂Q₁₀, —C(═NQ₁₀)Q₁₀, —C(═NOQ₁₀)Q₁₀, —S(O)₂—N═S(O)(Q₁₀)₂, —S(O)₂—N═S(Q₁₀)₂, —NQ₁₀Q₁₀, —C(O)Q₁₀, —C(S)Q₁₀, —C(O)OQ₁₀, —OC(O)Q₁₀, —C(O)NQ₁₀Q₁₀, —C(O)C(Q₁₆)₂OC(O)Q₁₀, —CN, —NQ₁₀C(O)Q₁₀, —N(Q₁₀)C(S)NQ₁₀Q₁₀, —N(Q₁₀O)C(S)Q₁₀, —NQ₁₀C(O)NQ₁₀Q₁₀, —S(O)₂NQ₁₀Q₁₀, —NQ₁₀S(O)₂Q₁₀, —NQ₁₀S(O)Q₁₀, —NQ₁₀SQ₁₀, —NO₂, —SNQ₁₀Q₁₀, alkyl, substituted alkyl, alkenyl, alkynyl, het, halo, cycloalkyl, cycloalkenyl, and aryl. The het, cycloalkyl, cycloalkenyl, alkenyl, alkynyl, and aryl being optionally substituted with 1-3 substitutuents selected from halo and Q₁₅.

The term “substituted het” refers to a het moiety including 1-4 substituents selected from —OQ₁₀, —SQ₁₀, —S(O)₂Q₁₀, —S(O)Q₁, —OS(O)₂Q₁₀, —C(═NQ₁₀Q₁₀, —C(═NOQ₁₀)Q₁₀, —S(O)₂—N═S(O)(Q₁₀)₂, —S(O)₂—N═S(Q₁₀)₂, —NQ₁₀Q₁₀, —C(O)Q₁₀, —C(S)Q₁₀, —C(O)OQ₁₀, —OC(O)Q₁₀, —C(O)NQ₁₀Q₁₀, —C(O)C(Q₁₆)₂OC(O)Q₁₀, —CN, ═O, ═S, —NQ₁₀C(O)Q₁₀, —NQ₁₀C(S)Q₁₀, —NQ₁₀C(O)NQ₁₀Q₁₀, —NQ₁₀C(S)NQ₁₀Q₁₀, —S(O)₂NQ₁₀Q₁₀, —NQ₁₀S(O)₂Q₁₀, —NQ₁₀S(O)Q₁₀, —NQ₁₀SQ₁₀, —NO₂, —SNQ₁₀Q₁₀, alkyl, substituted alkyl, het, halo, cycloalkyl, cycloalkenyl, and aryl. The het, cycloalkyl, cycloalkenyl, and aryl being optionally substituted with 1-3 substitutuents selected from halo and Q₁₅.

The term “substituted alkenyl” refers to a alkenyl moiety including 1-3 substituents —OQ₁₀, —SQ₁₀, —S(O)₂Q₁₀, —S(O)Q₁₀, —OS(O)₂Q₁₀, —C(═NQ₁₀)Q₁₀, —C(═NOQ₁₀)Q₁₀, —S(O)₂—N═S(O)(Q₁₀)₂, —S(O)₂—N═S(Q₁₀)₂, —NQ₁₀Q₁₀, —C(O)Q₁₀, —C(S)Q₁₀, —C(O)OQ₁₀, —OC(O)Q₁₀, —C(O)NQ₁₀Q₁₀, —C(S)NQ₁₀Q₁₀, —C(O)C(Q₁₆)₂OC(O)Q₁₀, —CN, ═O, ═S, ═NQ₁₀C(S)Q₁₀, —NQ₁₀C(O)Q₁₀, —NQ₁₀C(O)NQ₁₀Q₁₀, —NQ₁₀C(S)NQ₁₀Q₁₀, —S(O)₂NQ₁₀Q₁₀, —NQ₁₀S(O)₂Q₁₀, —NQ₁₀S(O)Q₁₀, —NQ₁₀SQ₁₀, —NO₂, —SNQ₁₀Q₁₀, alkyl, substituted alkyl, het, halo, cycloalkyl, cycloalkenyl, and aryl. The het, cycloalkyl, cycloalkenyl, and aryl being optionally substituted with 1-3 substitutuents selected from halo and Q₁₅.

The term “substituted alkoxy” refers to an alkoxy moiety including 1-3 substituents —OQ₁₀, —SQ₁₀, —S(O)₂Q₁₀, —S(O)Q₁₀, —OS(O)₂Q₁₀, —C(═NQ₁₀)Q₁₀, —C(═NOQ₁₀)Q₁₀, —S(O)₂—N═S(O)(Q₁₀)₂, —S(O)₂—N═S(Q₁₀)₂, —NQ₁₀Q₁₀, —C(O)Q₁₀, —C(S)Q₁₀, —C(O)OQ₁₀, —OC(O)Q₁₀, —C(O)NQ₁₀Q₁₀, —C(S)NQ₁₀Q₁₀, —C(O)C(Q₁₆)₂OC(O)Q₁₀, —CN, ═O, ═S, —NQ₁₀C(S)Q₁₀, —NQ₁₀C(O)Q₁₀, —NQ₁₀C(O)NQ₁₀Q₁₀, —NQ₁₀C(S)NQ₁₀Q₁₀, —S(O)₂NQ₁₀Q₁₀, —NQ₁₀S(O)₂Q₁₀, —NQ₁₀S(O)Q₁₀, —NQ₁₀SQ₁₀, —NO₂, —SNQ₁₀Q₁₀, alkyl, substituted alkyl, het, halo, cycloalkyl, cycloalkenyl, and aryl. The het, cycloalkyl, cycloalkenyl, and aryl being optionally substituted with 1-3 substitutuents selected from halo and Q₁₅.

The term “substituted cycloalkenyl” refers to a cycloalkenyl moiety including 1-3 substituents —OQ₁₀, 13 SQ₁₀, —S(O)₂Q₁₀, —S(O)Q₁₀, —OS(O)₂Q₁₀, —C(═NQ₁₀)Q₁₀, —C(═NOQ₁₀)Q₁₀, —S(O)₂—N═S(O)(Q₁₀)₂, —S(O)₂—N═S(Q₁₀)₂, —NQ₁₀Q₁₀, —C(O)Q₁₀, —C(S)Q₁₀, —C(O)OQ₁₀, —OC(O)Q₁₀, —C(O)NQ₁₀Q₁₀, —C(S)NQ₁₀Q₁₀, —C(O)C(Q₁₆)₂OC(O)Q₁₀, —CN, ═O, ═S, —NQ₁₀C(S)Q₁₀, —NQ₁₀C(O)Q₁₀, NQ₁₀C(O)NQ₁₀Q₁₀, —NQ₁₀C(S)NQ₁₀Q₁₀, —S(O)₂NQ₁₀Q₁₀, —NQ₁₀S(O)₂Q₁₀, —NQ₁₀S(O)Q₁₀, —NQ₁₀SQ₁₀, —NO₂, —SNQ₁₀Q₁₀, alkyl, substituted alkyl, het, halo, cycloalkyl, cycloalkenyl, and aryl. The het, cycloalkyl, cycloalkenyl, and aryl being optionally substituted with 1-3 substitutuents selected from halo and Q₁₅.

The term “substituted amino” refers to an amino moiety in which one or both of the amino hydrogens are replaced with a group selected from —OQ₁₀, —SQ₁₀, —S(O)₂Q₁₀, —S(O)Q₁₀, —OS(O)₂Q₁₀, —C(═NQ₁₀)Q₁₀, —C(═NOQ₁₀)Q₁₀, —S(O)₂—N═S(O)(Q₁₀)₂, —S(O)₂—N═S(Q₁₀)₂, —NQ₁₀Q₁₀, —C(O)Q₁₀, —C(S)Q₁₀, —C(O)OQ₁₀, —OC(O)Q₁₀, —C(O)NQ₁₀Q₁₀, —C(S)NQ₁₀Q₁₀, —C(O)C(Q₁₆)₂OC(O)Q₁₀, ═CN, ═O, ═S, —NQ₁₀C(O)Q₁₀, —NQ₁₀C(S)Q₁₀, —NQ₁₀C(O)NQ₁₀ Q₁₀, —NQ₁₀C(S)NQ₁₀Q₁₀, —S(O)₂NQ₁₀Q₁₀, —NQ₁₀S(O)₂Q₁₀, —NQ₁₀S(O)Q₁₀, —NQ₁₀SQ₁₀, —NO₂, —SNQ₁₀Q₁₀, alkyl, substituted alkyl, het, halo, cycloalkyl, cycloalkenyl, and aryl. The het, cycloalkyl, cycloalkenyl, and aryl being optionally substituted with 1-3 substitutuents selected from halo and Q₁₅.

Each Q₁₀ is independently selected from —H, alkyl, cycloalkyl, het, cycloalkenyl, and aryl. The het, alkyl, cycloalkyl, cycloalkenyl, and aryl being optionally substituted with 1-3 substitutuents selected from halo and Q₁₃.

Each Q₁₁ is independently selected from —H, halo, alkyl, aryl, cycloalkyl, and het. The alkyl, aryl, cycloalkyl, and het being optionally substituted with 1-3 substituents independently selected from halo, —NO₂, —CN, ═S, ═O, and Q₁₄.

Each Q₁₃ is independently selected from Q₁₁, —OQ₁₁, —SQ₁₁, —S(O)₂Q₁₁, —S(O)Q₁₁, —OS(O)₂Q₁₁, —C(═NQ₁₁)Q₁₁, —S(O)₂—N═S(O)(Q₁₁)₂, —S(O)₂—N═S(Q₁₁)₂, —SC(O)Q₁₁, —NQ₁₁Q₁₁, —C(O)Q₁₁, —C(S)Q₁₁, —C(O)OQ₁₁, —OC(O)Q₁₁, —C(O)NQ₁₁Q₁₁, —C(S)NQ₁₁Q₁₁, —C(O)C(Q₁₆)₂OC(O)Q₁₀, —CN, ═O, ═S, —NQ₁₁C(O)Q₁₁, —NQ₁₁C(S)Q₁₁, —NQ₁₁C(O)NQ₁₁Q₁₁, —NQ₁₁C(S)NQ₁₁Q₁₁, —S(O)₂NQ₁₁Q₁₁, —NQ₁₁S(O)₂Q₁₁, —NQ₁₁S(O)Q₁₁, —NQ₁₁SQ₁₁, —NO₂, and —SNQ₁₁Q₁₁.

Each Q₁₄ is —H or a substituent selected from alkyl, cycloalkyl, phenyl, or naphthyl, each optionally substituted with 1-4 substituents independently selected from —F, —Cl, —Br, —I, —OQ₁₆, —SQ₁₆, —S(O)₂Q₁₆, —S(O)Q₁₆, —OS(O)₂Q₁₆, —NQ₁₆Q₁₆, —C(O)Q₁₆, —C(S)Q₁₆, —C(O)OQ₁₆, —NO₂, —C(O)NQ₁₆Q₁₆, —C(S)NQ₁₆Q₁₆, —CN, —NQ₁₆C(O)Q₁₆, —NQ₁₆C(S)Q₁₆, —NQ₁₆C(O)NQ₁₆Q₁₆, —NQ₁₆C(S)NQ₁₆Q₁₆, —S(O)₂NQ₁₆Q₁₆, and —NQ₁₆S(O)₂Q₁₆. The alkyl, cycloalkyl, and cycloalkenyl being furher optionally substituted with ═O or ═S.

Each Q₁₅ is alkyl, cycloalkyl, heterocycloalkyl, heteroaryl, phenyl, or naphthyl, each optionally substituted with 1-4 substituents independently selected from —F, —Cl, —Br, —I, —OQ₁₆, —SQ₁₆, —S(O)₂Q₁₆, —S(O)Q₁₆, —OS(O)₂Q₁₆, —C(═NQ₁₆)Q₁₆, —S(O)₂—N═S(O)(Q₁₆)₂, —S(O)₂—N═S(Q₁₆)₂, —SC(O)Q₁₆, —NQ₁₆Q₁₆, —C(O)Q₁₆, —C(S)Q₁₆, —C(O)OQ₁₆, —OC(O)Q₁₆, —C(O)NQ₁₆Q₁₆, —C(S)NQ₁₆Q₁₆, —C(O)C(Q₁₆)₂OC(O)Q₁₆, —CN, —NQ₁₆C(O)Q)₁₆, —NQ₁₆C(S)Q)₁₆, —NQ₁₆C(O)NQ₁₆Q₁₆, —NQ₁₆C(S)NQ₁₆Q₁₆, —S(O)₂NQ₁₆Q₁₆, —NQ₁₆S(O)₂Q₁₆, —NQ₁₆S(O)Q₁₆, —NQ₁₆SQ₁₆, —NO₂, and —SNQ₁₆Q₁₆. The alkyl, cycloalkyl, and cycloalkenyl being furher optionally substituted with ═O or ═S.

Each Q₁₆ is independently selected from —H, alkyl, and cycloalkyl. The alkyl and cycloalkyl optionally including 1-3 halos. Mammal denotes human and animals.

Each Q₇ is independently selected from —H, —OH, and alkyl optionally including 1-3 halos and —OH.

The term “electron withdrawing group” refers to the ability of a substituent to withdraw electrons relative to that of hydrogen if the hydrogen atom occupied the same position on the molecule. The term “electron withdrawing group” is well understood by one skilled in the art and is discussed in Advanced Organic Chemistry by J. March, John Wiley & Sons, New York, N.Y., (1985) and the discussion therein is incorporated herein by reference. Electron withdrawing groups include, but are not limited to, groups such as halo, nitro, carboxy, cyano, aryl optionally substituted, aromatic het (excluding pyridine) optionally substituted, —OC(Zₙ)₃, —C(Zₙ)₃, —C(Zₙ)₂—O—C(Zₘ)₃, —(CO)—Q₁₇, —SO₂—C(Zₙ)₃, —SO₂-aryl, —C(NQ₁₇)Q₁₇, —CH═C(Q₁₇)₂, —C≡C—Q₁₇, in which each Zn and Zm is independently H, halo, —CN, —NO₂—OH, or C₁₋₄alkyl optionally substituted with 1-3 halo, —OH, NO₂, and provided that at least one of Zn is halo, —CN, or NO₂, and further provided that Q₁₇ is not —OH when the the electron withdrawing group is —(CO)-Q₁₇.

It is to be understood that the present invention encompasses any racemic, optically-active, polymorphic, tautomeric, or stereoisomeric form, or mixture thereof, of a compound of the invention, which possesses the useful properties described herein.

In cases where compounds are sufficiently basic or acidic to form stable nontoxic acid or base salts, use of the compounds as pharmaceutically acceptable salts may be appropriate. Examples of pharmaceutically acceptable salts which are within the scope of the present invention include organic acid addition salts formed with acids which form a physiological acceptable anion and inorganic salts. Examples of pharmaceutically acceptable salts include, but are not limited to, the following acids acetic, aspartic, benzenesulfonic, benzoic, bicarbonic, bisulfuric, bitartaric, butyric, calcium edetate, camsylic, carbonic, chlorobenzoic, citric, edetic, edisylic, estolic, esyl, esylic, formic, fumaric, gluceptic, gluconic, glutamic, glycollylarsanilic, hexamic, hexylresorcinoic, hydrabamic, hydrobromic, hydrochloric, hydroiodic, hydroxynaphthoic, isethionic, lactic, lactobionic, maleic, malic, malonic, mandelic, methanesulfonic, methylnitric, methylsulfuric, mucic, muconic, napsylic, nitric, oxalic, p-nitromethanesulfonic, pamoic, pantothenic, phosphoric, monohydrogen phosphoric, dihydrogen phosphoric, phthalic, polygalactouronic, propionic, salicylic, stearic, succinic, sulfamic, sulfanilic, sulfonic, sulfuric, tannic, tartaric, teoclic toluenesulfonic, primary, secondary, and tertiary amines, substituted amines including naturally occurring substituted amines, cyclic amines, such as arginine, betaine, caffeine, choline, N,N-dibenzylethylenediamine, diethylamine, 2-diethylaminoethanol, 2-dimethylamino-ethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, and the like.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

The antibacterial agents of this invention have useful activity against a variety of organisms. The in vitro activity of compounds of this invention can be assessed by standard testing procedures such as the determination of minimum inhibitory concentration (MIC) by agar dilution as described in “Approved Standard. Methods for Dilution Antimicrobial Susceptibility Tests for Bacteria That Grow Aerobically”, 3rd. ed., published 1993 by the National Committee for Clinical Laboratory Standards, Villanova, Pa., USA.

The antibacterial agents described herein are useful for sterilization, sanitation, antisepsis, and disinfection. The antibacterial agents can be applied to a location in need of sterilization, sanitation, antisepsis, or disinfection, by methods known to those skilled in the art. For instance, the antibacterial agents may be incorporated into a cleaning solution that is applied, such as by spraying or pouring, to an item in need of sterilization, sanitation, antisepsis, or disinfection. The antibacterial agents may be used alone or in combination, e.g., agents disclosed herein with one another or agent(s) disclosed herein with other antibacterial agents. The antibacterial agents may be applied in varying concentrations depending upon the bacterial susceptibility to antibacterial agent(s) being applied and the desired level of sterilization, sanitation, antisepsis, or disinfection.

The antibacterial compounds of this invention may be synthesized by various methods known to those skilled in the art. Non-limiting examples of synthetic schemes for producing the antibacterial agents are described below.

### EXAMPLES

Without further elaboration, it is believed that one skilled in the art can, using the preceding description, practice the present invention to its fullest extent. The following detailed examples describe how to prepare the various compounds and/or perform the various processes of the invention and are to be construed as merely illustrative, and not limitations of the preceding disclosure in any way whatsoever. Those skilled in the art will promptly recognize appropriate variations from the procedures both as to reactants and as to reaction conditions and techniques.

### Example 1

### Sulfonyl Derivatives

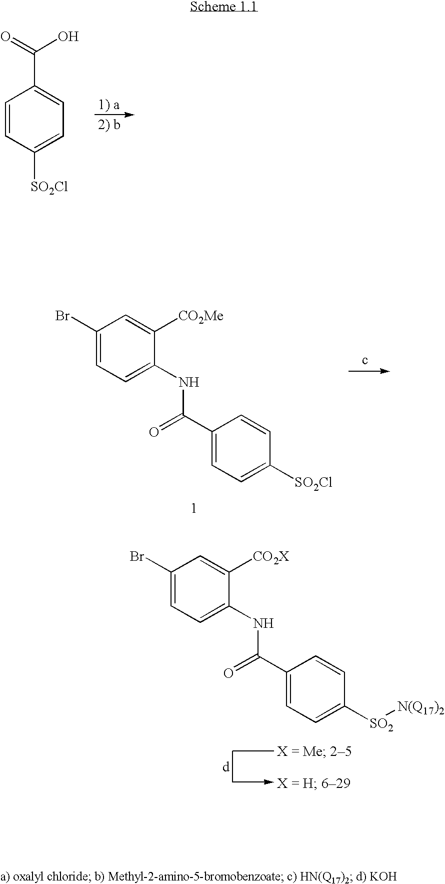

### Methyl 5-bromo-2-{[4-(chlorosulfonyl)benzoyl]amino}benzoate

Methyl 5-bromo-2-{[4-(chlorosulfonyl)benzoyl]amino}benzoate (1) was prepared as a common intermediate for the formation of sulfonamides by the procedure below: 4-(chlorosulfonyl)benzoic acid (18.37 g, 8.33 mmol) was suspended in CH₂Cl₂ (140 mL) and 4 drops of DMF. The solution was cooled to 0° C. and oxalyl chloride (1.8 mL, 20.6 mmol) was added and stirred for 1 hour, removed from ice bath, and stirred overnight. The clear solution was concentrated in vacuo, redissolved in CH₂Cl₂, and concentrated in vacuo. The resulting product was dissolved in toluene (140 mL) and refluxed for 30 minutes to remove any HCl gas. After cooling to room temperature, methyl-2-amino-5-bromobenzoate (15.96 g, 69.4 mmol) was added, and the suspension was refluxed overnight. The suspension was cooled to 0° C. and filtered, washing with toluene and quickly with ethyl acetate. The solid was dried in a vacuum oven overnight to obtain sulfonyl chloride 1 (19.8 g, 66%). ¹H NMR (CDCl₃) δ 12.19, 8.82, 8.27-8.19, 7.73, 4.00; IR 1700, 1683, 1604, 1585, 1524 (s), cm⁻¹; MS (ESI—) for C₁₅H₁₁BrClNO₅S m/z 429.8 (M−H)⁻.

General Method A (Sulfonamide Preparation with Anilines, Primary, and Secondary Amines)

### Methyl 5-bromo-2-({4-[(diethylamino)sulfonyl]benzoyl}amino)benzoate

To a solution of the sulfonyl chloride 1, (694.1 mg, 1.61 mmol, 1.0 eq) in toluene (4.0 mL) was added diethyl amine (500 μL, 4.83 mmol, 3.0 eq). The suspension was shaken at 50° C. for overnight. The product was extracted with EtOAc, washed with 1 N HCl and water, and concentrated in vacuo. The compound was dried in a vacuum oven at 50° C. overnight to obtain 624.4 mg (83%). ¹H NMR (300 MHz, DMSO-d₆) δ 11.48, 8.31, 8.11, 8.05, 7.99, 7.87, 3.86, 3.20, 1.04; IR 1700, 1676 (s), 1600, 1519 (s), 1338, 1330, 1306 (s), cm⁻¹. Anal. Calcd for C₁₉H₂₁BrN₂O₅S: C, 48.62; H, 4.51; N, 5.97; Br, 17.02; S, 6.83. Found: C, 48.76; H, 4.53; N, 5.89; Br, 16.98; S, 6.73.

General Method B (Hydrolysis of the Methyl Ester)

### 5-bromo-2-({4-[(diethylamino)sulfonyl]benzoyl}amino)benzoate, 8

Methyl 5-bromo-2-({4-[(diethylamino)sulfonyl]benzoyl}amino)benzoate (329.6 mg, 0.704 mmol) was dissolved in 2 mL of dioxane and 0.2 mL of water. KOH (1 pellet, ˜80 mg) was added to the mixture as it was heated at 50° C. for 3 hours. The reaction was cooled, extracted with EtOAc, washed with 1 N HCl and brine, dried (Na₂SO₄), concentrated in vacuo, and dried in a vacuum oven at 50° C. overnight to yield 313.8 mg (98%). ¹H NMR (300 MHz, DMSO-d₆) δ 12.05, 8.55, 8.11, 8.09, 8.00, 7.86, 3.19, 1.04; IR 1703, 1661, 1202, 1185, cm⁻¹. MS (FAB) m/z (rel. intensity) 455 (MH⁺, 45), 457 (37), 455 (45), 240 (99). HRMS (FAB) calcd for C₁₈H₁₉BRN₂O₅S+H₁ 455.0276, found 455.0260. Anal. Calcd for C₁₈H₁₉BrN₂O₅S: C, 47.48; H, 4.21; N, 6.15; Br, 17.55; S, 7.04. Found: C, 47.31; H, 4.25; N, 6.12.

5-bromo-2-({4-[(dimethylamino)sulfonyl]benzoyl}amino)benzoic acid 6, was prepared by method B from its methyl ester, i.e., Methyl 5-bromo-2-({4-[(dimethylamino)sulfonyl]benzoyl}amino)benzoate, in a 47% yield. ¹H NMR (300 MHz, CDCl₃) δ 8.89, 8.31, 8.18, 7.96, 7.78, 2.78; IR 3135, 1700, 1350 (s), 1191 (s), cm⁻¹. MS (ESI—) for C₁₆H₁₅BrNO₅S m/z 426.9 (M−H, Br isotope)⁻. Anal. Calcd for C₁₆H₁₅BrN₂O₅S: C, 44.98; H, 3.54; N, 6.56; Br, 18.70; S, 7.50. Found: C, 44.82; H, 3.55; N, 6.46; Br, 18.43; S, 7.36.

5-bromo-2-({4-[(1H-indol-5-ylamino)sulfonyl]benzoyl}amino)benzoate 7, was prepared by general method B from PNU-263551 in a 52% yield. ¹H NMR (300 MHz, DMSO-d₆) δ 12.05 (s, 1 H), 11.05 (s, 1 H), 10.00 (s, 1 H), 8.52 (d, J=9 Hz, 1 H), 8.10 (d, J=2 Hz, 1 H), 8.02 (d, J=8 Hz, 2 H), 7.85 (m, 3 H), 7.30 (t, J=1 Hz, 1 H), 7.25440 (s, 1 H), 7.24 (d, J=9 Hz, 1 H), 6.82 (dd, J=9, 1 Hz, 1 H), 6.34 (s, 1 H); IR 1687, 1664, 1607, 1524, 1338, 1314, 1300, 1189, 1170 (s), 825, 801, 756, 743, 681, 616 (s), cm⁻¹. MS (FAB) m/z (rel. intensity) 514 (MH⁺, 55), 516 (59), 515 (67), 514 (55), 132 (99), 131 (97). HRMS (FAB) calcd for C₂₂H₁₆BRN₃O₅S+H₁ 514.0073, found 514.0066. HPLC [1] shows one main peak at 16.3 min (95%). Anal. Calcd for C₂₂H₁₆BrN₃O₅S: C, 51.37; H, 3.13; N, 8.17; Br, 15.53; S, 6.23. Found: C, 51.16; H, 3.23; N, 8.01.

5-bromo-2-[(4-{[(3-furylmethyl)amino]sulfonyl}benzoyl)amino]benzoate 9, was prepared by method B from PNU-276173 in a 48% yield. ¹H NMR (300 MHz, DMSO-d₆) δ 8.60 (d, J=9 Hz, 1 H), 8.41 (t, J=6 Hz, 1 H), 8.14 (d, J=2 Hz, 1 H), 8.07 (d, J=8 Hz, 2 H), 7.93 (d, J=8 Hz, 2 H), 7.87 (dd, J=9, 2 Hz, 1 H), 7.46 (s, 1 H), 6.28 (s, 1 H), 6.18 (s, 1 H), 4.08 (d, J=6 Hz, 2 H); IR 3252, 1702, 1172 (s), 1165 (s), cm⁻¹. MS (FAB) m/z (rel. intensity) 479 (MH⁺, 13), 481 (14), 479 (13), 135 (99), 73 (64). HRMS (FAB) calcd for C₁₉H₁₅BRN₂O₆S+H₁ 478.9913, found 478.9922. Anal. Calcd for C₁₉H₁₅BrN₂O₆S: C, 47.61; H, 3.15; N, 5.84; Br, 16.67; S, 6.69. Found: C, 47.55; H, 3.22; N, 5.69; Br, 16.26; S, 6.60.

5-bromo-2-[(4-{[4-(ethoxycarbonyl)-1-piperazinyl]sulfonyl}benzoyl)amino]benzoic acid 10 was prepared by method A followed by B with a 26% yield over both steps. The methyl ester was not fully characterized. ¹H NMR (300 MHz, DMSO-d₆) δ 8.60 (d, J=9 Hz, 1 H), 8.18 (d, J=8 Hz, 2 H), 8.13 (d, J=2 Hz, 1 H), 7.94 (d, J=8 Hz, 2 H), 7.79 (dd, J=9, 2 Hz, 1 H), 3.97 (q, J=7 Hz, 2 H), 3.45 (br. s, 4 H), 2.95 (br. s, 4 H), 1.12 (t, J=7 Hz, 3 H); IR 1692 (s), 1675 (s), 1584, 1518 (s), 1287, 1276, 1250, cm⁻¹. MS (FAB) m/z (rel. intensity) 540 (MH⁺, 46), 542 (44), 540 (46), 159 (95), 157 (99). HRMS (FAB) calcd for C₂₁H₂₂BRN₃O₇S+H₁ 540.0440, found 540.0428. HPLC [1] shows one major peak at 16.2 min (97%). Anal. Calcd for C₂₁H₂₂BrN₃O₇S: C, 46.67; H, 4.10; N, 7.78; Br, 14.79; S, 5.93. Found: C, 46.34; H, 4.19; N, 7.63; Br, 14.18; S, 5.79.

5-bromo-2-{[4-({methyl[2-(2-pyridinyl)ethyl]amino}sulfonyl)benzoyl]amino}benzoic acid 11 was prepared by method A followed by B with a 57% yield over both steps. The methyl ester was not fully characterized. ¹H NMR (300 MHz, DMSO-d₆) δ 12.19 (s, 1 H), 8.58 (d, J=9 Hz, 1 H), 8.52 (d, J=4 Hz, 1 H), 8.13 (d, J=3 Hz, 1 H), 8.12 (d, J=6 Hz, 2 H), 7.96 (d, J=8 Hz, 2 H), 7.87 (dd, J=9, 2 Hz, 1 H), 7.78 (td, J=8, 2 Hz, 1 H), 7.35 (d, J=8 Hz, 1 H), 7.30 (td, J=6, 2 Hz, 1 H), 3.42 (t, J=7 Hz, 2 H), 2.99 (t, J=8 Hz, 2 H), 2.77 (s, 3 H); IR 1692 (s), 1518 (s), 1340 (s), 1297 (s), 1162 (s), 763 (s), 755 (s), 747 (s) cm⁻¹. MS (ES—) for C₂₂H₂₀BrN₃O₅S m/z 518.0 (M−H⁺, Br isotope); HRMS (FAB) calcd for C₂₂H₂₀BRN₃O₅S+H₁ 518.0386, found 518.0388. HPLC [1] shows one major peak (13.58 min, 99%).

2-({4-[(benzylamino)sulfonyl]benzoyl}amino)-5-bromobenzoic acid 12 was prepared by method A followed by B with a 17% yield over both steps. The methyl ester was not fully characterized. ¹H NMR (300 MHz, DMSO-d₆) δ 12.09 (s, 1 H), 8.60 (d, J=9 Hz, 1 H), 8.39 (t, J=6 Hz, 1 H), 8.14 (d, J=2 Hz, 1 H), 8.08 (d, J=8 Hz, 2 H), 7.97 (d, J=8 Hz, 2 H), 7.88 (dd, J=9, 2 Hz, 1 H), 7.30-7.20 (m, 5 H), 4.05 (d, J=6 Hz, 2 H); HRMS (FAB) calcd for C₂₁H₁₇BRN₂O₅S+H₁ 489.0120, found 489.0129; HPLC [1] shows one major peak (20.60 min, 99%).

5-bromo-2-[(4-{[(2-hydroxy-1-methylethyl)amino]sulfonyl}benzoyl)amino]benzoic acid 14 was prepared by method A followed by B with a 35% yield over both steps. The methyl ester was not fully characterized. ¹H NMR (300 MHz, DMSO-d₆) δ 8.56 (d, J=9 Hz, 1 H), 8.11 (d, J=2 Hz, 1 H), 8.09 (d, J=8 Hz, 2 H), 8.00 (d, J=8 Hz, 2 H), 7.86 (dd, J=9, 2 Hz, 1 H), 7.76 (d, J=7 Hz, 1 H), 3.26 (m, 2 H), 3.12 (m, 1 H), 0.89 (d, J=6 Hz, 3 H); MS (ES—) for C₁₇H₁₇BrN₂O₆S m/z 454.9 (M−H⁺); HPLC [1] shows one major peak (14.08 min, 96%).

5-bromo-2-({4-[(4-carboxyanilino)sulfonyl]benzoyl}amino)benzoic acid 15 was prepared from method A followed by method B in a 10% yield. The methyl ester was not fully characterized. ¹H NMR (300 MHz, DMSO-d₆) δ 12.45 (br. s, 1 H), 11.15 (s, 1 H), 8.52 (d, J=9 Hz, 1 H), 8.08 (d, J=8 Hz, 3 H), 8.03 (d, J=9 Hz, 2 H), 7.81 (d, J=9 Hz, 3 H), 7.26 (d, J=9 Hz, 2 H); HPLC [1] shows one major peak (15.15 min, 90%).

5-bromo-2-{[4-(3,4-dihydro-1(2H)-quinolinylsulfonyl)benzoyl]amino}benzoic acid 16 was prepared by method A followed by method B in a 48% yield. The methyl ester was not fully characterized. ¹H NMR (300 MHz, DMSO-d₆) δ 12.05 (s, 1 H), 8.52 (d, J=9 Hz, 1 H), 8.11 (d, J=3 Hz, 1 H), 8.05 (d, J=9 Hz, 2 H), 7.86 (dd, J=9, 2 Hz, 1 H), 7.82 (d, J=8 Hz, 2 H), 7.61 (d, J=8 Hz, 1 H), 7.25-7.05 (m, 3 H), 3.83 (t, J=6 Hz, 2 H), 2.45 (t, J=7 Hz, 2H), 1.63 (quintet, J=6 Hz, 2 H); IR 1667, 1601, 1584, cm⁻¹. HRMS (FAB) calcd for C₂₃H₁₉BRN₂O₅S+H₁ 515.0276, found 515.0264. Anal. Calcd for C₂₃H₁₉BrN₂O₅S: C, 53.60; H, 3.72; N, 5.43. Found: C, 53.52; H, 3.96; N, 5.57.

5-bromo-2-{[4-({[2-(3,5-dimethoxyphenyl)ethyl]amino}sulfonyl)benzoyl]amino}benzoic acid 17 was prepared by method A followed by B with a 56% yield over both steps. The methyl ester was not fully characterized. ¹H NMR (300 MHz, DMSO-d₆) δ 8.60 (d, J=9 Hz, 1 H), 8.13 (d, J=3 Hz, 1 H), 8.09 (d, J=8 Hz, 2 H), 7.95 (d, J=9 Hz, 2 H), 7.87 (dd, J=9, 2 Hz, 1 H), 6.79 (d, J=8 Hz, 1 H), 6.73 (d, J=2 Hz, 1 H), 6.64 (dd, J=8, 2 Hz, 1 H), 3.70 (s, 3 H), 3.68 (s, 3 H), 3.02 (q, J=6 Hz, 2 H), 2.61 (t, J=7 Hz, 2 H); MS (FAB) m/z (rel. intensity) 563 (MH⁺, 86), 565 (86), 564 (82), 563 (86), 562 (56), 348 (77), 199 (46), 165 (56), 164 (32), 152 (49), 151 (99). HRMS (EI) calcd for C₂₄H₂₃BRN₂O₇S 562.0410, found 562.0438. HPLC [1] shows one major peak (16.16 min, 97%).

5-bromo-2-[(4-{[(3S)-3-hydroxypyrrolidinyl]sulfonyl}benzoyl)amino]benzoic acid 13 was prepared by method A followed by B in a 15% yield over both steps. The methyl ester was not fully characterized. ¹H NMR (300 MHz, DMSO-d₆) δ 8.62 (d, J=9 Hz, 1 H), 8.18 (d, J=8 Hz, 2 H), 8.11 (d, J=3 Hz, 1 H), 7.92 (d, J=11 Hz, 2 H), 7.78 (dd, J=9, 2 Hz, 1 H), 5.16 (m, 1 H), 3.50-3.20 (m, 4 H), 2.10-1.90 (m, 2 H); HPLC [1] shows one major peak (18.94 min, 97%).

5-bromo-2-({4-[(ethylanilino)sulfonyl]benzoyl}amino)benzoic acid 19 was prepared by method A followed by B with a 75% yield over both steps. The methyl ester was not fully characterized. ¹H NMR (300 MHz, CD₃OD) δ 8.75 (d, J=9 Hz, 1 H), 8.24 (d, J=2 Hz, 1 H), 8.11 (d, J=8 Hz, 2 H), 7.76 (dd, J=9, 2 Hz, 1 H), 7.74 (d, J=8 Hz, 2 H), 7.34 (m, 3 H), 7.06 (m, 2 H), 3.69 (q, J=7 Hz, 2 H), 1.07 (t, J=7 Hz, 3 H); MS (ES—) for C₂₂H₁₉BrN₂O₅S m/z 502.8 (M−H⁺; Br isotope); HRMS (FAB) calcd for C₂₂H₁₉BRN₂O₅S+H₁ 503.0276, found 503.0265. HPLC [1] shows one major peak (18.60 min, 99%).

5-bromo-2-({4-[(3,5-dimethoxyanilino)sulfonyl]benzoyl}amino)benzoic acid 20 was prepared by method A followed by B with a 69% yield over both steps. The methyl ester was not fully characterized. ¹H NMR (300 MHz, CD₃OD) δ 8.73 (d, J=9 Hz, 1 H), 8.24 (d, J=2 Hz, 1 H), 8.09 (d, J=9 Hz, 2 H), 7.96 (d, J=9 Hz, 2 H), 7.74 (dd, J=9, 2 Hz, 1 H), 6.32 (s, 1 H), 6.31 (s, 1 H), 6.20 (s, 1 H), 3.70 (s, 6 H); MS (ES—) for C₂₂H₁₉BrN₂O₇S m/z 532.8 (M−H⁺); HPLC [1] shows one major peak (17.06 min, 96%).

5-bromo-2-[(4-{[(2-hydroxy-2-phenylethyl)(methyl)amino]sulfonyl}benzoyl)amino]benzoic acid 21 was prepared by method A followed by B with a 15% yield over both steps. The methyl ester was not fully characterized. ¹H NMR (300 MHz, CD₃OD) δ 12.10 (s, 1 H), 8.57 (d, J=9 Hz, 1 H), 8.12 (d, J=2 Hz, 1 H), 8.11 (d, J=9 Hz, 2 H), 7.95 (d, J=8 Hz, 2 H), 7.87 (dd, J=9, 3 Hz, 1 H), 7.35-7.27 (m, 5 H), 4.76 (t, J=7 Hz, 1 H), 3.22-3.13 (m, 2 H), 2.77 (s, 3 H); MS (FAB) m/z (rel. intensity) 533 (MH⁺, 61), 535 (64), 533 (61), 517 (99), 516 (24), 515 (90), 318 (46), 152 (27), 134 (25), 132 (33), 44 (44). HRMS (FAB) calcd for C₂₃H₂₁BRN₂O₆S+H₁ 533.0382, found 533.0386. HPLC [1] shows one majorpeak (17.06, 97%).

5-bromo-2-{[4-(2,3-dihydro-1H-indol-1-ylsulfonyl)benzoyl]amino}benzoic acid 22 was prepared by method A followed by B in a 55% yield over both steps. The methyl ester was not fully characterized. ¹H NMR (300 MHz, DMSO-d₆) δ 12.00 (s, 1 H), 8.51 (d, J=9 Hz, 1 H), 8.10-8.01 (m, 5 H), 7.84 (dd, J=9, 3 Hz, 1 H), 7.50 (d, J=8 Hz, 1 H), 7.22 (t, J=8 Hz, 1 H), 7.17 (d, J=8 Hz, 1 H), 7.00 (1, J=7 Hz, 1 H), 3.98 (t, J=8 Hz, 2 H), 2.93 (t, J=8 Hz, 2 H); IR 1687, 1667, 1601, 1525 (s), 1365 (s), 1245 (s), 1172 (s), cm⁻¹. MS (FAB) m/z (rel. intensity) 501 (MH⁺, 36), 503 (41), 502 (43), 501 (36), 500 (31), 286 (35), 118 (99). HRMS (FAB) calcd for C₂₂H₁₇BRN₂O₅S+H₁ 501.0120, found 501.0118. Anal. Calcd for C₂₂H₁₇BrN₂O₅S: C, 52.71; H, 3.42; N, 5.59; Br, 15.94; S, 6.39. Found: C, 52.65; H, 3.47; N, 5.58; Br, 15.88; S, 6.24.

5-bromo-2-({4-[(5-methoxy-2,3-dihydro-1H-indol-1-yl)sulfonyl]benzoyl}amino)benzoic acid 23 was prepared by method A followed by B in a 17% yield over both steps. The methyl ester was not fully characterized. ¹H NMR (300 MHz, DMSO-d₆) δ 12.05 (s, 1 H), 8.51 (d, J=9 Hz, 1 H), 8.10 (d, J=2 Hz, 1 H), 8.05 (d, J=8 Hz, 2 H), 7.95 (d, J=9 Hz, 2 H), 7.86 (dd, J=9, 2 Hz, 1 H), 7.42 (d, J=9 Hz, 1 H), 6.78 (d, J=8 Hz, 1 H), 6.77 (s, 1 H), 3.96 (t, J=8 Hz, 2 H), 3.68 (s, 3 H), 2.80 (t, J=8 Hz, 2 H); IR 1702, 1606, 1518, 1489 (s), 1358, 1199 (s), 1168 (s), cm⁻¹. MS (FAB) m/z (rel. intensity) 531 (MH⁺, 29), 533 (30), 531 (29), 530 (38), 148 (99). HRMS (EI) calcd for C₂₃H₁₉BRN₂O₆S 530.0148, found 530.0156. Anal. Calcd for C₂₃H₁₉BrN₂O₆S: C, 51.99; H, 3.60; N, 5.27; Br, 15.04; S, 6.03. Found: C, 52.08; H, 3.61; N, 5.29.

5-bromo-2-({4-[(5-fluoro-2,3-dihydro-1H-indol-1-yl)sulfonyl]benzoyl}amino)benzoic acid 24 was prepared by method A followed by B with a 41% yield over both steps. The methyl ester was not fully characterized. ¹H NMR (300 MHz, DMSO-d₆) δ 12.05 (s, 1 H), 8.51 (d, J=9 Hz, 1 H), 8.10 (d, J=2 Hz, 1 H), 8.07 (d, J=9 Hz, 2 H), 7.99 (d, J=9 Hz, 2 H), 7.85 (dd, J=9, 2 Hz, 1 H), 7.49 (dd, J=10, 5 Hz, 1 H), 7.07-7.02 (m, 2 H), 4.01 (t, J=8 Hz, 2 H), 2.89 (t, J=8 Hz, 2 H); MS (ES—) for C₂₂H₁₆BrN₂O₅S m/z 518.9 (M−H⁺, Br isotope); HPLC [2] shows one major peak (6.35 min, 96%).

2-{[4-(1H-benzimidazol-1-ylsulfonyl)benzoyl]amino}-5-bromobenzoic acid 26 was prepared from method A followed by hydrolysis of the methyl ester by the hydrolysis procedure in method C below. ¹H NMR (300 MHz, DMSO-d₆) δ 11.98 (s, 1 H), 8.91 (s, 1 H), 8.47 (d, J=9 Hz, 1 H), 8.41 (d, J=9 Hz, 2 H), 8.13 (d, J=9 Hz, 2 H), 8.09 (d, J=2 Hz, 1 H), 7.93 (d, J=7 Hz, 1 H), 7.85 (dd, J=9, 3 Hz, 1 H), 7.78 (d, J=7 Hz, 1 H), 7.47 (t, J=6 Hz, 1 H), 7.40 (t, J=6 Hz, 1 H); IR 1686, 1607, 1522, 1391, 1296, 1262, 1190, cm⁻¹. MS (ESI—) for C₂₁H₁₄BrN₃O₅S m/z 497.7 (M−H)⁻. HPLC [2] shows one major peak at 6.01 min (96%). Anal. Calcd for C₂₁H₁₆BrN₃O₅S: C, 50.21; H, 3.21; N, 8.36; Br, 15.91; S, 6.38. Found: C, 50.06; H, 2.85; N, 7.93; Br, 15.34; S, 6.22.

General Method C (Sulfonamide Preparation with Indoles and Pyrrole):

Reaction of sulfonyl chloride intermediate 1 with indole derivatives requires modified conditions. Deprotonation of the indole nitrogen with sodium hydride in THF and reaction with the sulfonyl chloride 1 provided the desired intermediate methyl esters. Two equivalents of the indole anion were required because of competitive deprotonation of the amide in 1. Attempted hydrolysis of such methyl esters with aqueous KOH results in hydrolysis of the newly formed sulfonamide. Therefore, dealkylative deesterification conditions were utilized (Scheme 1.2).

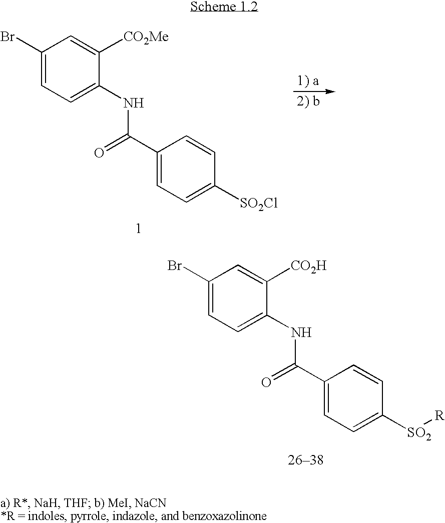

5-bromo-2-({4-[(5-fluoro-1H-indol-1-yl)sulfonyl]benzoyl}amino)benzoic acid 26 was prepared by the following procedure: 5-fluoroindole (497.1 mg, 3.68 mmol, 2.2 eq) was dissolved in anhydrous THF (8 mL) and cooled to 0° C. NaH (60% dispersion in mineral oil, 150 mg, 3.75 mmol, 2.2 eq) was added and the cloudy mixture was stirred for 1 hr. at 0-25° C. The suspension was then cooled to 0° C. and Methyl 5-bromo-2-{[4-(chlorosulfonyl)benzoyl]amino}benzoate (722.0 mg, 1.68 mmol, 1.0 eq) was added neat and stirred overnight at room temperature. After quenching with water, the product was extracted with EtOAc and washed with 1 N HCl, concentrated in vacuo, triturated with MeOH, filtered and washed with MeOH. A mixture of the carboxylic acid and ester (469.0 mg) was obtained. The mixture of products were both committed to the hydrolysis conditions: 4 mL dioxane, 0.4 mL water, and 1 KOH pellet (˜90 mg) were added to the mixture of acid and ester and shook at 50° C. for 3 hrs. The hydrolysis was monitored by HPLC. The product was dissolved in EtOAc and washed with 1 N HCl, concentrated in vacuo, triturated with MeOH, filtered, and washed with MeOH to obtain 246.8 mg (28%) of 5-bromo-2-({4-[(5-fluoro-1H-indol-1-yl)sulfonyl]benzoyl}amino)benzoic acid. ¹H NMR (300 MHz, DMSO-d₆) δ 11.95 (s, 1 H), 8.43 (d, J=9 Hz, 1 H), 8.19 (d, J=9 Hz, 2 H), 8.07 (d, J=3 Hz, 1 H), 8.05 (d, J=9 Hz, 2 H), 7.96 (dd, J=9, 4 Hz, 1 H), 7.91 (d, J=4 Hz, 1 H), 7.82 (dd, J=9, 2 Hz, 1 H), 7.42 (dd, J=9, 3 Hz, 1 H), 7.20 (td, J=9, 3 Hz, 1 H), 6.86 (d, J=4 Hz, 1 H); IR (drift) 1692, 1670, 1601, 1524 (s), 1462, 1388 (s), 1290, 1242, 1234, 1218 (s), 1181 (s), 1140 (s), 742,649 (s), 607 (s), cm⁻¹. MS (ESI—) for C₂₂H₁₄BrFN₂O₅S m/z 516.9 (M−H, Br isotope)⁻. HPLC [2] shows one major peak at 6.56 min (98%). Anal. Calcd for C₂₂H₁₄BrFN₂O₅S: C, 51.08; H, 2.73; N, 5.41; Br, 15.44; S, 6.20. Found: C, 51.05; H, 2.64; N, 5.39.

Other compounds were prepared by the above procedure making non-critical variations.

5-bromo-2-{[4-(1H-indol-1-ylsulfonyl)benzoyl]amino}benzoic acid, 5-bromo-2-({4-[(6-fluoro-1H-indol-1-yl)sulfonyl]benzoyl}amino)benzoic acid, 5-bromo-2-({4-[(5-chloro-1H-indol-1-yl)sulfonyl]benzoyl}amino)benzoic acid, 5-bromo-2-({4-[(6-chloro-1H-indol-1-yl)sulfonyl]benzoyl}amino)benzoic acid, 5-bromo-2-({4-[(6-chloro-5-fluoro-1H-indol-1-yl)sulfonyl]benzoyl}amino)benzoic acid, 5-bromo-2-{[4-(1H-pyrrol-1-ylsulfonyl)benzoyl]amino}benzoic acid, 5-bromo-2-({4-[(5-methoxy-1H-indol-1-yl)sulfonyl]benzoyl}amino)benzoic acid, 5-bromo-2-{[4-(1H-pyrrolo[2,3-]pyridin-1-ylsulfonyl)benzoyl]amino}benzoic acid

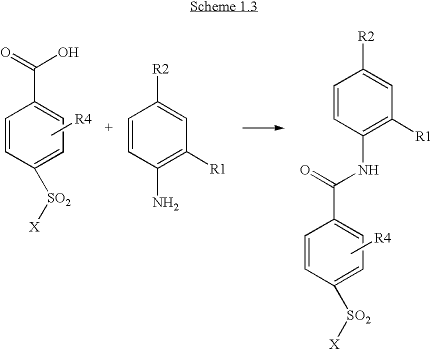

### Preparation of Methyl 3-bromo-5-[(5-bromo-2,3-dihydro-1H-indol-1-yl)sulfonyl]benzoate

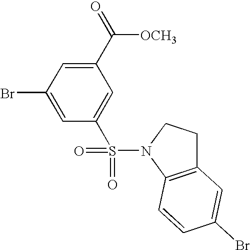

A solution of 5-bromoindoline (528 mg, 2.67 mmol, Lancaster) and triethylamine (650 μL, 4.67 mmol) in CH₂Cl₂ (8 mL) was added to a solution of methyl 3-bromo-5-chlorosulfonyl)benzoate (737 mg, 2.35 mmol) in CH₂Cl₂ (10 mL). The mixture was stirred overnight and then diluted to 100 mL with CH₂Cl₂. This solution was washed with 2×100 mL of 1 M aqueous HCl and 100 mL of brine. The CH₂Cl₂ was evaporated in the presence of silica gel, and the product was purified by chromatography using a Biotage Flash 40 M silica cartridge with a gradient from 50% CH₂Cl₂/heptane to 75% CH₂Cl₂/heptane as eluent. Yield was 945 mg of pale yellow solid.

### Preparation of 3-Bromo-5-[(5-bromo-2,3-dihydro-1H-indol-1-yl)sulfonyl]benzoic acid

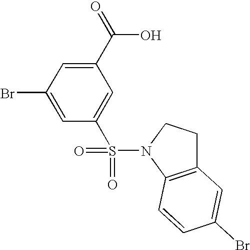

To a mixture of the corresponding methyl ester (841 mg, 1.77 mmol) in methanol (20 mL) was added 1 M aqueous sodium hydroxide (3.0 mL). The mixture was stirred in a 50° C. oil bath for 10 minutes and then at 60° C. for 15 minutes. The mixture was still a slurry, so 10 mL of dioxane was added. Heat was removed after an additional 40 minutes. The reaction mixture was added to a separatory funnel with 100 mL of 1 M aqueous HCl, and the product was extracted into 100 mL of CH₂Cl₂. The organics were washed with an additional 100 mL of 1 M aqueous HCl followed by 100 mL of water. They were then dried over MgSO₄ and evaporated yielding 807 mg of white solid.

### Methyl 5-bromo-2-({3-bromo-5-[(5-bromo-2,3-dihydro-1H-indol-1-l)sulfonyl]benzoyl}amino)benzoate

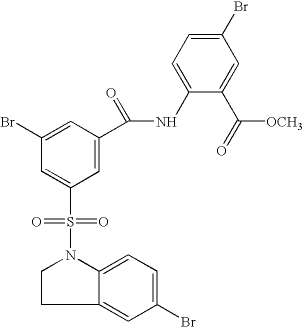

To 3-bromo-5-[(5-bromo-2,3-dihydro-1H-indol-1-yl)sulfonyl]benzoic acid (583 mg, 1.26 mmol) in CH₂Cl₂ (25 mL) was added DMF (20 μL) and oxalyl chloride (220 μL, 2.52 mmol). The mixture was stirred for 1 hour, and the solvent and excess oxalyl chloride were removed by rotary evaporation. The residue was dissolved in CH₂Cl₂ (10 mL), and methyl 2-amino-5-bromobenzoate (267 mg, 1.16 mmol, Avocado) in pyridine (4 mL) was added. The mixture was stirred overnight and then added to a separatory funnel with 100 mL of CH₂Cl₂. Some THF was added to help solubility. This mixture was washed with 2×100 mL of 1 M aqueous HCl and 100 mL of brine. The organics were evaporated, and the residue was dissolved in hot THF. This solution was evaporated in the presence of silica gel, and the product was purified by chromatography using a Biotage Flash 40 M silica cartridge with a gradient from 50% CH₂Cl₂/heptane to 100% CH₂Cl₂ as eluent. Yield was 603 mg of white solid.

General Method D: (hydrolysis of the methyl ester)

### 5-Bromo-2-({3-bromo-5-[(5-bromo-2,3-dihydro-1H-indol-1-yl)sulfonyl]benzoyl}amino)benzoic acid

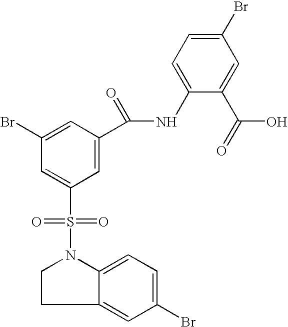

To a mixture of the corresponding methyl ester (374 mg, 0.556 mmol) in dioxane (30 mL) was added 1 M aqueous sodium hydroxide (1.1 mL). The mixture was stirred in a 60° C. oil bath for 90 minutes. The reaction mixture was added to a separatory funnel with 100 mL of 1 M aqueous HCl, and the product was extracted into 100 mL of CH₂Cl₂. The organics were washed with an additional 100 mL of 1 M aqueous HCl followed by 100 mL of water. They were then dried over MgSO₄ and evaporated. The residue was recrystallized from hot ethanol/THF. The solids were washed with ethanol and then dried at 100° C. under vacuum yielding 266 mg of white solid. ¹H NMR (400 MHz, DMSO-d₆) δ 12.14 (s, 1 H), 8.48 (d, J=8.7 Hz, 1 H), 8.36 (s, 1 H), 8.31 (s, 1 H), 8.19 (s, 1 H), 8.12 (d,J=2.0 Hz, 1 H), 7.86 (dd, J=8.7, 2.5 Hz, 1 H), 7.39-7.49 (m, 3 H), 4.04 (t, J=8.4 Hz, 2 H), 2.99 (t, J=8.4 Hz, 2 H).

### Preparation of Methyl 4-[(5-chloro-2,3-dihydro-1H-indol-1-yl)sulfonyl]benzoate

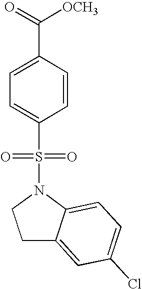

To 4-[(5-chloro-2,3-dihydro-1H-indol-1-yl)sulfonyl]benzoic acid (456 mg, 1.35 mmol) in CH₂Cl₂ (30 mL) was added DMF (15 μL) and oxalyl chloride (150 μL, 1.72 mmol). The mixture was stirred for 5 hours, and the solvent and excess oxalyl chloride were removed by rotary evaporation. The residue was dissolved in CH₂Cl₂ (10 mL). Methanol (2 mL) and pyridine (2 mL) in CH₂Cl₂ (6 mL) were added. The mixture was stirred for 30 minutes and then added to a separatory funnel with 100 mL of CH₂Cl₂. This solution was washed with 100 mL of 1 M aqueous HCl, 100 mL of saturated aqueous NaHCO₃, another 100 mL of HCl, and 100 mL of brine. The CH₂Cl₂ was dried over MgSO₄ and evaporated yielding 464 mg of white solid.

### Preparation of {4-[(5-Chloro-2,3-dihydro-1H-indol-1-yl)sulfonyl]phenyl}methanol

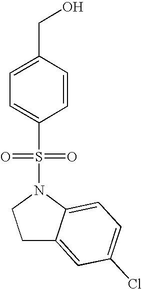

To a solution of methyl 4-[(5-chloro-2,3-dihydro-1H-indol-1-yl)sulfonyl]benzoate (396 mg, 1.13 mmol) in THF (20 mL) was added lithium borohydride (0.40 mL of 2.0 M solution in THF, 0.80 mmol, Aldrich). HPLC analysis after 1.5 hours indicated <10% reaction, so lithium aluminum hydride (0.60 mL of 1.0 M solution in THF, Aldrich) was added at −78 ° C. The mixture was stirred at −78 ° C. for 15 minutes and then warmed to room temperature. The reaction was quenched by the addition of water (25 μL) followed by 6 M aqueous NaOH (25 μL) followed by another portion of water (75 μL). The mixture was filtered, and the filtrate was evaporated in the presence of silica gel. The product was purified by chromatography using a Biotage Flash 40 M silica cartridge with a gradient from CH₂Cl₂ to 10% EtOAC in CH₂Cl₂ as eluent. Yield was 290 mg of white solid.

### Preparation of t-butyl 2-nitrobenzoate

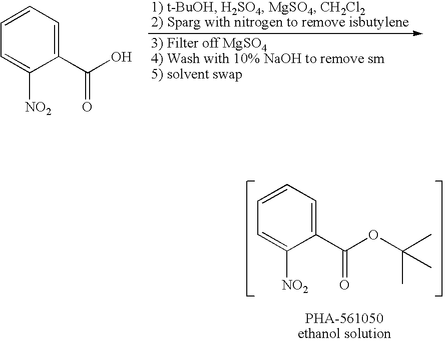

A 22 L round bottom flask, equipped with an mechanical stirrer, thermocouple, and a 1 L addition funnel, was charged with 500 g (2.99 moles, 1.0 equiv) of 2-nitrobenzoic acid (Avocado Research Chemicals Ltd, 98%) and 1.44 kg (11.97 moles, 4 equiv) of anhydrous magnesium sulfate (EM Science, 98%). To the solids were charged 12.5 L (25 mL/g) of CH₂Cl₂ (EM Science, 99.96%) and 1.43 L (2.99 moles, 1.0 equiv) of t-butyl alcohol (Aldrich, 99+% A.C.S. Reagent). The addition funnel was charged with 1.59 mL (2.99 moles, 1.0 equiv) of concentrated sulfuric acid (Mallinckrodt, 95.7%) and the entire system was sealed via use of a Teflon cap (loose fit; internal pressure does not exceed 11 psi; theory=10.5 psi). The resulting suspension was cooled to 16° C. using a water bath and 159 mL (2.99 moles, 1.0 equiv) of concentrated sulfuric acid was added at a rate of 2.8 mL/min, maintaining an internal temperature less than 25° C. The resulting off-white suspension was stirred at room temperature for 14 hours at which time the HPLC assay indicated the reaction was at 92% conversion. The suspension was sparged with nitrogen for 15 min using ½ inch ID Teflon tubing and filtered through a sintered glass funnel (course) with the aid of house vacuum (ca. 16 torr; filtration time of 1.0 h). The cake was rinsed with CH₂Cl₂ (500 mL, 1 mL/g). The combined filtrates were charged to a 30 L wash tank and diluted with 2 L of water (pH=1.0). To the resulting biphasic mixture was added 2.5 L of 10% NaOH over a 15 min period (8° C. exotherm; pH=12.0). The resulting yellow-colored aqueous layers were separated from the clear, colorless organic layer. The organic layer was concentrated in vacuo at 16 torr using a 37° C. water bath to provide a 93% yield (621 g, 2.78 moles) as a light yellow oil. To ensure removal of residual CH₂Cl₂, the oil was dissolved in 2 L of absolute ethanol (AAPER, 200 proof) and concentrated in vacuo at 16 torr using a 57° C. water bath. The potency of the material was determined to be 99.2% (GC) and 99.0% (HPLC) and was taken on directly to the next step without further purification.

### Preparation of t-butyl 2-aminobenzoate

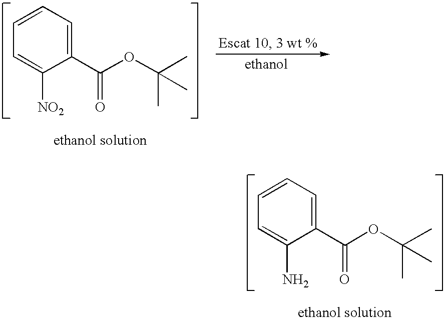

Escat 10 catalyst (18.63 g, 3 wt %) was charged to the 10 L autoclave followed by t-butyl nitrobenzoate (621 g, 2.78 moles) in ethanol (7 L). The vessel was sealed and purged three times with nitrogen (60 psig) and three times with hydrogen (60 psig). The vessel was then pressurized to 50 psig with hydrogen and allowed to run holding the exotherm at 40° C. through external cooling. The reaction was run until the hydrogen uptake stopped (45 minutes). The reaction was determined to be complete by both TLC and HPLC after 1 h and 10 min. The reaction was filtered through a 0.4 μ filter to remove the catalyst, and the catalyst cake was rinsed with ethanol (1.5 L). The product solution was then concentrated in vacuo at 16 torr using a 45° C. water bath to a volume of 1620 mL (3 mL/g) and taken on directly into the next step. An aliquot of the solution was concentrated and analyzed by both NMR and GC. The GC potency of the final product was 100%, and the NMR spectra were consistent with the structure of the title compound.

### Preparation of t-butyl 2-amino-5-iodobenzoate

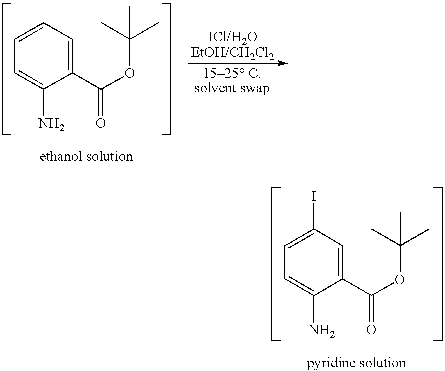

A 12 L round bottom flask, equipped with a thermocouple, nitrogen adapter and a 1 L addition funnel, was charged with a solution of t-butyl 2-aminobenzoate (537 g, 2.78 moles; lot 36648-tjb-40) in ethanol (1620 ml, 3 ml/g). To this golden solution was added water (615.6 ml) resulting in a biphasic mixture. This mixture was cooled to between 15 and 20° C. with a cold-water bath. A 1.0 M solution of ICl in CH₂Cl₂ (Aldrich lot #14127JO, 3.11 L, 3.11 moles, 1.12 equiv.) was charged in portions to the addition funnel and was added to the rapidly stirred mixture maintaining the temperature between 15 and 25° C. The addition time was 2.25 hours and the temperature range observed was 16.5 to 20.4° C. The resulting red brown mixture was stirred at room temperature for 1 hour at which time the GC assay showed the reaction was complete. The reaction was diluted with 920 mL of water and quenched with 456 mL of 38% aq. sodium bisulfite (Webb Chem lot #10464519) resulting in a slight exotherm to 24.0° C. This mixture was stirred for 15 minutes before separating the phases. The methylene chloride layer was combined with water (3.7 L) and stirred for 15 minutes before separating the phases. A NaOH solution was prepared by diluting 10% NaOH (460 ml) in water (2.3 L). To the methylene chloride layer was added this dilute NaOH solution (2.1 L). The pH of the basic phase was 6.56. The phases were separated and the methylene chloride layer was concentrated to a low volume in vacuo at 16 torr using a bath temp of 45° C. Pyridine (4 L) was added, and the resulting solution was concentrated to ca. 1.0 mL/g in vacuo at 16 torr using a 62° C. water bath. The low volume pyridine/product mixture was diluted with pyridine to the target volume of 3.1 L (3.5 mL/g). A sample (10 mL) was concentrated removing the pyridine on the rotovap and high vacuum to yield 3.12 g of an orange brown solid of 96% potecy by GC. GC assay of pyridine solution indicated that neither EtOH nor methylene chloride were present, so the solution was taken on directly into the next step.

### Preparaion of t-butyl 2-amino-5-cyanobenzoate

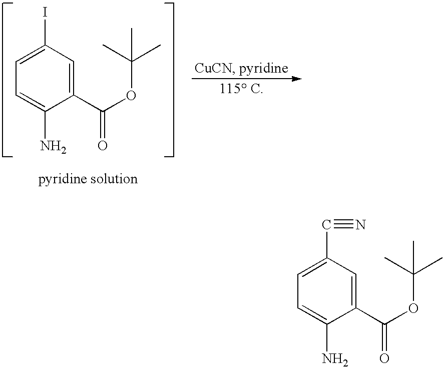

A 5 L Morton flask equipped with a mechanical stirrer (sturdy blade), thermocouple, and a reflux condenser was charged with 299 g (3.34 moles, 1.2 equiv) of CuCN (Aldrich, 99%). To the slowly stirred CuCN was added a cool (10° C.) solution of t-butyl 2-amino-5-iodobenzoate (887 g, 2.78 moles, 1.0 equiv) in pyridine (3.5 mL/g including the volume occupied by t-butyl 2-amino-5-iodobenzoate). The resulting orange suspension was heated to 115° C. over 45 min to produce a black solution. The solution was maintained at 115° C. for 14 h at which point GC indicated the reaction was complete. The solution was cooled to 90° C. and transferred by ½ inch ID Teflon cannula to a stirred suspension of solka floc (powdered cellulose, 460 g) in 14 L of methyl-tert-butyl ether (EM Science, 99.95%) at 2° C., maintaining an internal temperature less than 13° C. The resulting yellow-green suspension was filtered through a sintered glass frit (course frit, 16 torr vacuum) and the cake was rinsed with 4 L of MTBE (EM Science, 99.95%). The filtrate was washed (1×8 L H₂O, 3×2 L of 10% NH₄OH in 23% NH₄Cl), and the organics were concentrated in vacuo at 16 torr using a 50° C. water bath to a volume of 3 L (3.4 mL/g). The solution was split in half and crystallized in two portions. One half of the solution was charged to a 22 L flask containing heptanes (8 L). The flask was set up for atmospheric distillation and heptanes (4 L) was added to bring total volume of heptanes to 12 L. The mixture was distilled atmospherically to remove 4 L of distillate (pot temp of 98° C.; head temp of 96° C.). The pot was charged with 4 L of heptanes, and another 4 L of distillate was removed. A second 4 L charge of heptanes was made and 2.4 L of distillate was removed via atmospheric distillation; thus reducing the pot volume to 8.9 L (20 mL/g). GC assay of the final distillate indicated the following volume percent ratios of pyridine and MTBE, respectively: 2.08% and 1.51%. The heating mantle was removed, and the solution was cooled to induce crystallization (crystal formation was first noted at about 56° C.). The slurry was stirred at room temperature for 4 h, and the solids were isolated by vacuum filtration on a 3 L frit. The cake was slurry washed with room temperature heptanes (2×500 ml) and dried on a nitrogen press to produce 224.2 g of an off-white solid (GC potency of 100%). Crystallization of the second half of the material produced another 241 g; thus a 70% yield from 2-nitrobenzoic acid was achieved.

An alternative methodology for producing t-butyl 2-amino-5-cyanobenzoate is shown below.

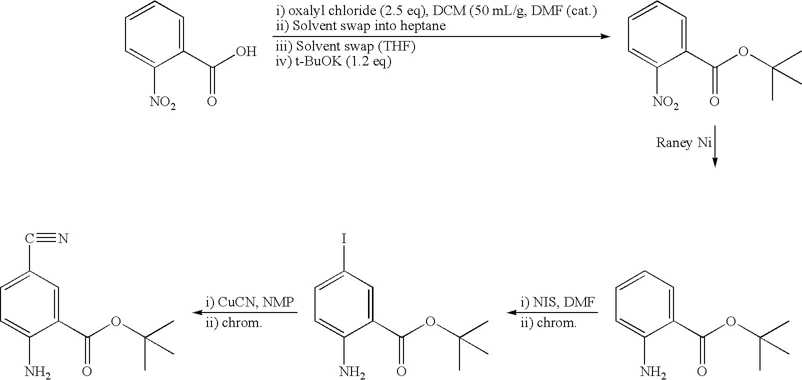

### 5-Cyano-2-({3-[(3,3-dimethyl-2,3-dihydro-1H-indol-1-yl)sulfonyl]benzoyl}amino)benzoic acid

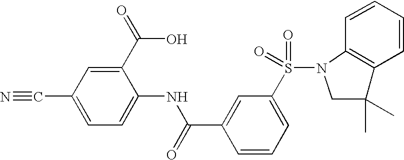

To a solution of 3-(chlorosulfonyl)benzoic acid (456 mg, 2.07 mmol, Aldrich) in CH₂Cl₂ (15 mL) was added DMF (15 μL) followed by oxalyl chloride (270 μL, 3.10 mmol). After stirring for 1.5 hours, the solvent and excess oxalyl chloride were removed by rotary evaporation. The residue was dissolved in toluene (15 mL), and methyl 2-amino-5-cyanobenzoate (370 mg, 2.10 mmol) was added. The mixture was heated in a 105° C. oil bath for 2 hours, and the toluene was then removed by rotary evaporation. The residue was dissolved in CH₂Cl₂ (6 mL), and a mixture of 3,3-dimethylindoline, descrinbed by Kucerovy et al. in Synth. Commun. 1992, 22(5), 729-733, (342 mg, 2.32 mmol) and triethylamine (600 μL, 4.31 mmol) in CH₂Cl₂ (6 mL) was added. This mixture was stirred overnight and then added to a separatory funnel with 100 mL of CH₂Cl₂. This solution was washed with 2×100 mL of 1 M aqueous HCl and 100 mL of brine. The CH₂Cl₂ was evaporated in the presence of silica gel, and the product was purified by chromatography using a Biotage Flash 40 M silica cartridge with a gradient from CH₂Cl₂ to 1% EtOAc in CH₂Cl₂ as eluent. Yield was 728 mg of white solid as the methyl ester. The methyl ester was hydrolyzed according to method D yielding 292 mg of white solid. ¹H NMR (400 MHz, DMSO-d₆) δ 12.57 (s, 1 H), 8.80 (d, J=8.7 Hz, 1 H), 8.41-8.44 (m, 2 H), 8.24 (d, J=7.9 Hz, 1 H), 8.09-8.14 (m, 2 H), 7.83 (t, J=7.9 Hz, 1 H), 7.55 (d, J=8.1 Hz, 1 H), 7.24 (t, J=7.7 Hz, 1 H), 7.18 (d, J=7.7 Hz, 1 H), 7.02 (t, J=7.5 Hz, 1 H), 3.73 (s, 2 H), 1.08 (s, 6 H).

### 5-Bromo-2-[(4-{[(4-chlorophenyl)(methyl)amino]sulfonyl}benzoyl)amino]benzoic acid

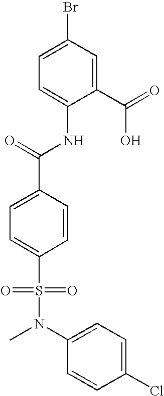

Dimethyl formamide (15 μL) and oxalyl chloride (1.5 mL, 17 mmol) were added sequentially to a mixture of 4-{[(4-chlorophenyl)(methyl)amino]sulfonyl}benzoic acid (2.82 g, 8.66 mmol) in CH₂Cl₂ (60 mL). The resulting solution was stirred for 3 hours after which the solvent and excess oxalyl chloride were removed by rotary evaporation. The residue was dissolved in CH₂Cl₂ (50 mL), and methyl 2-amino-5-bromobenzoate (1.83 g, 7.95 mmol, Avocado) in pyridine (I5 mL) was added. The mixture was stirred overnight and then added to a separatory funnel with 150 mL of CH₂Cl₂. The resulting solution was washed with 2×100 mL of 1M aqueous HCl and 100 mL of brine. The CH₂Cl₂ was evaporated in the presence of silica gel, and the product was purified by chromatography using a Biotage Flash 40 s siliga cartridge with CH₂Cl₂ as the eluent. Product was isolated as 3.73 g (87%) of a white solid as the methyl ester. The methyl ester was hydrolyzed according to method B. ¹H NMR (400 MHz, DMSO-d₆) δ 12.12 (s, 1 H), 8.56 (d, J=8.7 Hz, 1 H), 8.10-8.14 (m, 3 H), 7.88 (dd, J=8.7, 2.5 Hz, 1 H), 7.74 (d, J=8.1 Hz, 2 H), 7.43 (d, J=8.7 Hz, 2 H), 7.18 (d, J=8.7 Hz, 2 H), 3.18 (s, 3 H).

### Preparation of 4-Bromo-3-(2,3-dihydro-1H-indol-1-ylsulfonyl)benzoic acid

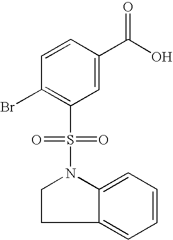

A solution of indoline (4.1 g, 34 mmol, Aldrich) and triethylamine (7.0 mL, 50 mmol) in methanol (20 mL) was added by cannula to solid 4-bromo-3-(chlorosulfonyl)benzoic acid (7.30 g, 24.4 mmol) with stirring in an ice bath. The mixture was allowed to warm slowly to room temperature and stirred overnight. It was added to a separatory funnel with 80 mL of aqueous 1 M NaOH, and this solution was washed with 2×100 mL of CH₂Cl₂. The aqueous layer was then acidified with concentrated HCl. The precipitate was washed with water followed by heptane and then recrystallized from toluene/ethanol. The crystals were washed with toluene followed by heptane and then dried at 100° C. under vacuum yielding 2.75 g of white solid. A second crop of 1.39 g of tan solid was also collected.

### Preparation of 4-Cyano-3-(2,3-dihydro-1H-indol-1-ylsulfonyl)benzoic acid

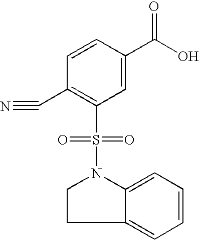

A mixture of copper (I) cyanide (755 mg, 8.43 mmol) and 4-bromo-3-(2,3-dihydro-1H-indol-1-ylsulfonyl)benzoic acid (2.05 g, 5.36 mmol) in NMP (15 mL) was heated to 160° C. under nitrogen for 1 hour. The mixture was added to a flask with 150 mL of EtOAc and 100 mL of water and stirred for 30 minutes. It was then filtered through a plug of celite. The phases were separated, and the water was extracted with an additional 2×100 mL of EtOAc. The combined EtOAc was washed with 3×100 mL of water and dried over MgSO₄. The solvent was removed, and the brown residue was recrystallized from hot ethanol. The crystals were washed with methanol followed by heptane and then dried at 100° C. under vacuum. Yield was 1.25 g of tan solid.

### 5-Bromo-2-{[4-cyano-3-(2,3-dihydro-1H-indol-1-ylsulfonyl)benzoyl]amino}benzoic acid

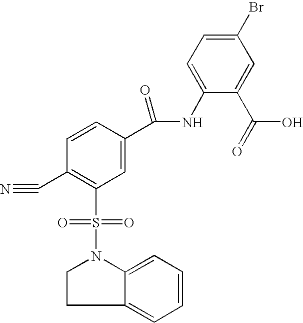

To 4-cyano-3-(2,3-dihydro-1H-indol-1-ylsulfonyl)benzoic acid (1.22 g, 3.72 mmol) in CH₂Cl₂ (30 mL) was added DMF (20 μL) and oxalyl chloride (650 μL, 7.45 mmol). The mixture was stirred for 2.3 hours, and the solvent and excess oxalyl chloride were removed by rotary evaporation. The residue was dissolved as best as possible in CH₂Cl₂ (30 mL), and methyl 2-amino-5-bromobenzoate ( 762 mg, 3.31 mmol, Avocado) in pyridine (15 mL) was added. The mixture was stirred overnight and then added to a separatory funnel with 100 mL of CH₂Cl₂. This solution was washed with 2×100 mL of 1 M aqueous HCl and 100 mL of brine. The CH₂Cl₂ was evaporated in the presence of silica gel, and the product was purified by chromatography using a Biotage Flash 40 M silica cartridge with CH₂Cl₂ as eluent. Yield was 1.31 g of yellow solid. The methyl ester was hydrolyzed according to Method D to yield 615 mg of yellow solid. ¹H NMR (400 MHz, DMSO-d₆) δ 12.24 (s, 1 H), 8.57 (s, 1 H), 8.51 (d, J=8.7 Hz, 1 H), 8.37 (d, J=7.6 Hz, 1 H), 8.32 (d, J=7.6 Hz, 1 H), 8.14 (d, J=2.5 Hz, 1 H), 7.88 (dd, J=8.9, 2.3 Hz, 1 H), 7.43 (d, J=8.1 Hz, 1 H), 7.16-7.24 (m, 2 H), 7.01 (t, J=7.6 Hz, 1 H), 4.20 (t, J=8.4 Hz, 2 H), 3.05 (t, J=8.4 Hz, 2 H).

### Preparation of Methyl 3-(chlorosulfonyl)-2-methylbenzoate

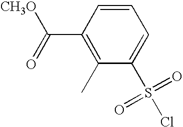

A flask was charged with methyl 2-methyl-3-nitrobenzoate (Aldrich, 5.0 g, 25.6 mmol) and tin (II) chloride dihydrate (28.9 g, 128 mmol, 5.0 eq). The solids were suspended in EtOAc (80 mL), and upon heating to reflux under N₂ the solids completely dissolved. After two hours the cooled reaction was poured into 350 mL EtOAc and then washed 4× with 1.0M NaOH, 1× with water and 1× with brine (350 mL each). The organic layer was dried over Na₂SO₄, filtered and the solvent evaporated. The resultant crude oil (2.9 g) was suspended in 60 mL of a 2:1 solution of concentrated HCl and glacial acetic acid. The reaction was cooled to −10° C. and a solution of sodium nitrite (1.33 g, 19.34 mmol) in 3.0 mL water was added drop wise over stirring at a rate that maintained the internal reaction temperature below −5° C. The reaction became an orange solution as the SM slowly dissolved. In a separate flask, copper (I) chloride (435 mg, 25 mol %) was suspended in 30 mL of a saturated (30% w/w) solution of sulfur dioxide gas in glacial acetic acid. The mixture was cooled on an ice bath over stirring, and after 2.5 hours the diazonium solution was added portion wise to the copper mixture over 15 minutes. The addition evolved gas and produced a lime green solution, which came to RT and was stirred overnight. The reaction was poured into ice water (200 mL) to afford an oil at the bottom of a pale blue solution. The solution was extracted 2× with CH₂Cl₂ (150 mL ea) and the organic phase was washed 2× with saturated NaHCO₃ and brine (250 mL ea). The golden organic solution was dried over Na₂SO₄, filtered and the solvent evaporated. The crude residue was purified on a Biotage Flash 40M+ (100 g) silica cartridge using a gradient of 20% heptane in CH₂Cl₂ to 100% CH₂Cl₂. The combined fractions were evaporated and the product was dried under high vacuum at RT to afford 2.2 g of pale pink solid. ¹H NMR (400 MHz, DMSO-d₆) δ 7.96 (dd, J=7.7, 1.5 Hz, 1 H), 7.59 (dd, J=7.7, 1.5 Hz, 1 H), 7.23 (t, J=7.7 Hz, 1 H), 3.82 (s, 3 H), 2.56 (s, 3 H).

### Preparation of 3-{[(4-chlorophenyl)(methyl)amino]sulfonyl}-2-methylbenzoate

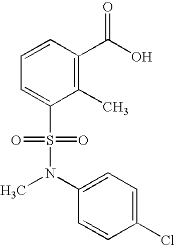

Methyl 3-(chlorosulfonyl)-2-methylbenzoate, (494 mg, 1.99 mmol) was taken up in dry CH₂Cl₂ (10 mL) and treated with 4-chloro-N-methylaniline (1.01 mL, 8.35 mmol, Aldrich) in dry pyridine (15 mL). The bright yellow solution was heated to 75° C. After one hour HPLC indicated the reaction was complete and the mixture was poured into EtOAc (125 mL). The organic phase was washed 3× with 1.0M HCl, 1× with saturated NaHCO₃ and 1× with brine (100 mL each). After drying over Na₂SO₄ the solution was filtered and the solvent was evaporated to afford an amber oil, which was purified on a Biotage Flash 40M+ (100 g) silica cartridge using a linear gradient of 35% to 5% heptane in CH₂Cl₂. The solvent was evaporated from the product fractions and the product was dried under high vacuum at RT to afford 637 mg (90%) of a colorless oil. 508 mg, 1.44 mmol of the oil was dissolved in MeOH (15 mL) and treated with 1.0M LiOH (3.0 mL, 3.0 mmol). After stirring at 40° C. for 1 hour and then overnight at RT, the reaction was complete by HPLC and OAMS showed the correct m/z for product. The reaction was poured into 1.0M HCl (100 mL), and the white precipitate was extracted into EtOAc (150 mL). The organic layer was then 1× with 1.0M HCl and 1× with brine (125 mL each). The organic layer was dried over MgSO₄, filtered and evaporated to dryness. The resultant product was dried under vacuum at 100° C. overnight to afford 461 mg (94%) of off-white solid. ¹H NMR (400 MHz, DMSO-d₆) δ 13.41 (br s, 1 H), 7.94 (d, J=3.3 Hz, 1 H), 7.92 (d, J=3.1 Hz, 1H), 7.49 (t, J=7.9 Hz, 1 H), 7.39-7.47 (m, 2 H), 7.22-7.31 (m, 2 H), 3.21 (s, 3 H), 2.45 (s, 3 H).

### 5-Bromo-2-[(3-{[(4-chlorophenyl)(methyl)amino]sulfonyl}-2-methylbenzoyl)amino]benzoic acid

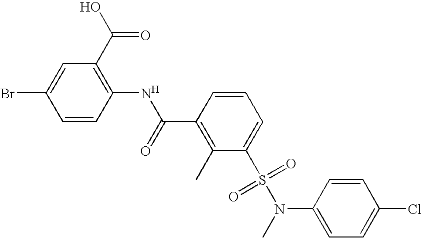

3-{[(4-chlorophenyl)(methyl)amino]sulfonyl}-2-methylbenzoate (404 mg, 1.19 mmol) was suspended in dry CH₂Cl₂ (10 mL) and DMF (10 μL) under N₂. The solution was treated with oxalyl chloride (Aldrich, 0.192 mL, 2.2 mmol) and stirred while gas evolved. After one hour the excess solvent and oxalyl chloride were evaporated and the resultant residue was taken up in dry CH₂Cl₂ (10 mL). Methyl-2-amino-5-bromobenzoate (Aldrich, 230 mg, 1.0 mmol) was added as a solution in pyridine (3 mL) and the amber solution stirred at RT. After 2 hours HPLC indicated the reaction was complete. The mixture was diluted with CH₂Cl₂ (100 mL) and washed 2× with 1.0M HCl followed by brine (100 mL each). The organic layer was evaporated and purified on a Biotage Flash 25M+ (40 g) silica cartridge using CH₂Cl₂. The combined fractions were evaporated and the product was dried under vacuum at 100° C. to afford 535 mg (97%) of a glass-like solid. ¹H NMR (400 MHz, DMSO-d₆) δ 10.88 (s, 1 H), 8.05 (d, J=8.9 Hz, 1 H), 7.99 (d, J=2.3 Hz, 1 H), 7.93 (D, J=7.5 Hz, 1 H), 7.86 (dd, J=8.8, 2.4 Hz, 1 H), 7.80 (d, J=7.3 Hz, 1 H), 7.57 (t, J=7.9 Hz, 1 H), 7.45 (d, J=8.7 Hz, 2 H), 7.29 (d, J=8.7 Hz, 2 H), 3.83 (s, 3 H), 3.24 (s, 3 H), 2.39 (s, 3 H). 322 mg of the methyl ester solid was dissolved in hot dioxane (10 mL), and after cooling was treated with 1.0M LiOH (1.0 mL, 1.0 mmol). After stirring overnight at RT the reaction was complete by HPLC and OAMS showed correct m/z for the product. The solvent was evaporated and the residue was poured into 1.0M HCl (100 mL) to afford a white precipitate. The product was extracted into EtOAc (125 mL) and washed 3× with 1.0M HCl, and 1× with brine (100 mL each). The organic layer was dried over Na₂SO₄, filtered and evaporated to dryness. The crude product was re-crystallized from hot MeOH/EtOH. The resultant product was dried at 100° C. under vacuum to afford 213 mg (68%) of white crystals. ¹H NMR (400 MHz, DMSO-d₆) δ 11.35 (s, 1 H), 8.39 (d, J=8.9 Hz, 1 H), 8.07 (d, J=2.5 Hz, 1 H), 7.92 (dd, J=8.1, 1.0 Hz, 1 H), 7.81-7.89 (m, 2 H), 7.56 (t, J=7.8 Hz, 1 H), 7.41-7.48 (m, 2 H), 7.24-7.34 (m, 2 H), 3.23 (s, 3 H), 2.39 (s, 3 H).

### Preparation of 3-[(5-Chloro-2,3-dihydro-1H-indol-1-yl)sulfonyl]-2-methylbenzoic acid

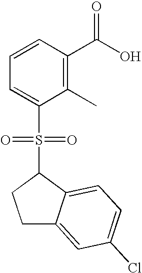

Methyl 3-(chlorosulfonyl)-2-methylbenzoate, (673 mg, 2.71 mmol) was taken up in dry CH₂Cl₂ (5 mL) and dry pyridine (5 mL). The golden solution was cooled to −10° C. and treated with 5-chloroindoline (1.01 mL, 8.35 rnmol, Aldrich) in dry CH₂Cl₂ (5 mL) to afford an intensely red-orange solution. A precipitate formed as the reaction warmed to RT. After one hour HPLC indicated the reaction was complete and the mixture was diluted to 150 mL with CH₂Cl₂. The organic phase was washed 1× with 1.0M HCl, 1× with 1.0M NaOH, 1× with 1.0M HCl and 1× with brine (125 mL each). After drying over Na₂SO₄ the solution was filtered and the solvent was evaporated. The resultant product was dried under high vacuum at RT to afford 900 mg (90%) of a peach colored oil. 780 mg (2.13 mmol) of the oil was dissolved in MeOH (15 mL) and treated with 1.0M LiOH (5.0 mL, 5.0 mmol). After stirring at 40° C. for 1 hour and then overnight at RT, the reaction was complete by HPLC and OAMS showed the correct m/z for product. The reaction was poured into 1.0M HCl (125 mL), and the yellowish precipitate was extracted into EtOAc (150 mL). The organic layer was then 2× with 1.0M HCl, 1× with water and 1× with brine (125 mL each). The organic layer was dried over MgSO₄, filtered and evaporated to dryness. The resultant product was dried under vacuum at 100° C. overnight to afford 711 mg (95%) of pinkish-orange solid. ¹H NMR (400 MHz, DMSO-d₆) δ 13.46 (br s, 1 H), 7.98 (d, J=8.1 Hz, 1 H), 7.93 (d, J=7.7 Hz, 1 H), 7.50 (t, J=7.9 Hz, 1 H), 7.34 (d, J=1.7 Hz, 1 H), 7.19 (dd, J=8.5, 2.1 Hz, 1 H), 7.09 (d, J=8.5 Hz, 1 H), 4.05 (t, J=8.5 Hz, 2 H), 3.12 (t, J=8.5 Hz, 2 H), 2.66 (s, 3 H).

### 2-({3-[(5-Chloro-2,3-dihydro-1H-indol-1-yl)sulfonyl]-2-methylbenzoyl}amino)-5-cyanobenzoic acid

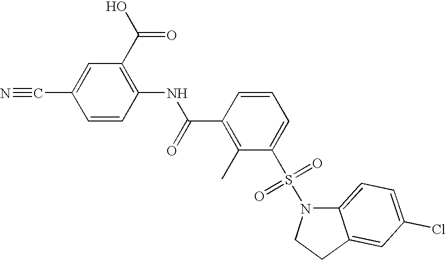

3-[(5-Chloro-2,3-dihydro-1H-indol-1-yl)sulfonyl]-2-methylbenzoic acid (553 mg, 01.57 mmol) was suspended in dry CH₂Cl₂ (15 mL) and DMF (10 μL) under N₂. The solution was treated with oxalyl chloride (0.274 mL, 3.14 mmol, Aldrich) and stirred while gas evolved. The reaction became homogenous and after one hour the excess solvent and oxalyl chloride was evaporated and the resultant residue was taken up in dry CH₂Cl₂ (10 mL). Methyl-2-amino-5-cyanobenzoate (PHA-522499, 264 mg, 1.5 mmol) was added as a solution in pyridine (4 mL) and the amber solution stirred at RT. After 2.5 days HPLC indicated the reaction was nearly complete. After briefly boiling the reaction and cooling, the mixture was diluted to 150 mL with CH₂Cl₂ and washed 2× with 1.0M HCl followed by brine (125 mL each). The organic layer was dried over Na₂SO₄, filtered and evaporated. The resultant crude product was purified on a Biotage Flash 25M+ (40 g) silica cartridge using a linear gradient of 0-2% EtOAc in CH₂Cl₂. The resultant product still contained a small amount of residual cyanoanthranilate. The combined fractions were evaporated and the product was purified a second time on a Biotage Flash 40M+ (100 g) silica cartridge using 100% CH₂Cl₂. The combined fractions were evaporated and dried under high vacuum at RT to afford 594 mg (77%) of an off-white solid. ¹H NMR (400 MHz, DMSO-d₆) δ 11.21 (s, 1 H), 8.29 (d, J=1.9 Hz, 1 H), 8.26 (d, J=8.7 Hz, 1 H), 8.11 (dd, J=8.6, 2.0 Hz, 1 H), 7.99 (dd, J=8.1, 1.0 Hz, 1 H), 7.84 (dd, J=7.7, 1.0 Hz, 1 H), 7.60 (t, J=7.9 Hz, 1 H), 7.36 (d, J=1.7 Hz, 1 H), 7.21 (dd, J=8.7, 2.3 Hz, 1 H), 7.16 (d, J=8.7 Hz, 1 H), 4.09 (t, J=8.6 Hz, 2 H), 3.84 (s, 3 H), 3.15 (t, J=8.4 Hz, 2 H), 2.61 (s, 3 H). The methyl ester was hydrolyzed as described above to afford 300 mg (77%) of white solid. ¹H NMR (400 MHz, DMSO-d₆) δ 11.73 (s, 1 H), 8.62 (d, J=8.7 Hz, 1 H), 8.36 (d, J=2.1 Hz, 1 H), 8.10 (dd, J=8.7, 2.1 Hz, 1 H), 7.97 (d, J=8.1 Hz, 1 H), 7.90 (d, J=6.8 Hz, 1 H), 7.57 (t, J=7.9 Hz, 1 H), 7.37 (s, 1 H), 7.20 (dd, J=8.7, 2.1 Hz, 1 H), 7.16 (d, J=8.5 Hz, 1 H), 4.07 (t, J=8.6 Hz, 2 H), 3.15 (t, J=8.5 Hz, 2 H), 2.62 (s, 3 H).

### Preparation of 3-{[(4-Chlorophenyl)(methyl)amino]sulfonyl}-2-methoxybenzoic acid

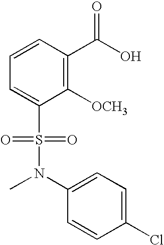

Methyl 3-amino-2-methoxybenzoate (1.27 g, 6.72 mmol) was dissolved in 30 mL of a 2:1 solution of concentrated HCl and glacial acetic acid. The reaction was cooled to −10° C. and a solution of sodium nitrite (696 mg, 10.1 mmol) in 3.0 mL water was added drop wise over stirring at a rate that maintained the internal reaction temperature below −5° C. The reaction became a cloudy yellow-orange suspension. In a separate flask, copper (I) chloride (166 mg, 25 mol %) was suspended in 30 mL of a saturated (30% w/w) solution of sulfur dioxide gas in glacial acetic acid. The mixture was cooled on an ice bath over stirring, and after 30 minutes diazonium solution was added portion wise to the copper mixture over 15 minutes. The addition evolved gas and produced a dark green solution. The reaction was warmed to RT and was stirred for 3 hours with sulfur dioxide bubbling into the solution. The reaction was poured into ice water (200 mL) to afford a fine white precipitate in a pale blue solution. The solution was extracted 3× with EtOAc (150 mL ea) and the organic phase was neutralized by washing 3× with saturated NaHCO₃ (300 mnL ea). The organic phase was then washed 2× with water and 1× with brine (250 mL ea). The golden organic solution was dried over Na₂SO₄, filtered and the solvent evaporated. The crude residue was dried under high vacuum to afford a dark red oil. The oil was taken up in pyridine (15 mL) and treated with 4-chloro-N-methylaniline (0.280 mL, 2.3 mmol, Aldrich). The amber solution was heated stirred at RT, and after one hour HPLC indicated the reaction was complete. The mixture was diluted to 150 mL with DCM and then washed 2× with 1.0M HCl, 1× with 1.0M NaOH and 1× with brine (125 mL each). The solvent was evaporated to afford an amber oil, which was purified on a Biotage Flash 40M (90 g) silica cartridge using a linear gradient of 0 to 0.75% EtOAc in CH₂Cl₂. The solvent was evaporated from the product fractions and the product was dried under high vacuum at RT to afford 614 mg (72%) of a straw colored oil as the methyl ester. The methyl ester was hydrolyzed as described above to afford 544 mg (97%) of peach colored solid. ¹H NMR (400 MHz, DMSO-d₆) δ 13.50 (s, 1 H), 7.99 (dd, J=7.7, 1.9 Hz, 1 H), 7.80 (dd, J=7.9, 1.7 Hz, 1 H), 7.36-7.42 (m, 2 H), 7.30 (t, J=7.9 Hz, 1 H), 7.19-7.26 (m, 2 H), 3.83 (s, 3 H), 3.32 (s, 3 H).

### 5-Bromo-2-[(3-{[(4-chlorophenyl)(methyl)amino]sulfonyl}-2-methoxybenzoyl)amino]benzoic acid

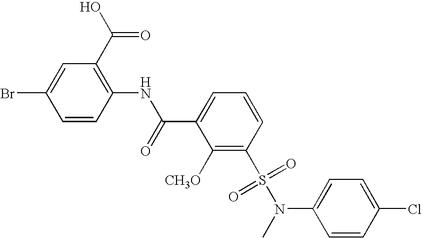

3-{[(4-Chlorophenyl)(methyl)amino]sulfonyl}-2-methoxybenzoic acid (PHA-733277, 474 mg, 01.33 mmol) was dissolved in dry CH₂Cl₂ (10 mL) and DMF (25 μL) under N₂. The solution was treated with oxalyl chloride (0.232 mL, 2.66 mmol, Aldrich) and stirred while gas evolved. The reaction was stirred at RT and after one hour the excess solvent and oxalyl chloride was evaporated and the resultant residue was taken up in dry CH₂Cl₂ (10 mL). Methyl-2-amino-5-bromobenzoate (288 mg, 1.25 mmol, Avocado) was added as a solution in pyridine (3 mL) and the amber solution stirred at RT. After 90 minutes HPLC indicated the reaction was complete. The mixture was diluted to 150 mL with CH₂Cl₂ and washed 2× with 1.0M HCl followed by brine (100 mL each). The organic layer was dried over Na₂SO_{4,} filtered and evaporated. The resultant crude product was purified on a Biotage Flash 40M (90 g) silica cartridge using CH₂Cl₂. The combined fractions were evaporated and dried under vacuum at 100° C. to afford 530 mg (72%) of an off-white solid as the methyl ester. ¹H NMR (400 MHz, DMSO-d₆) δ 11.52 (s, 1 H), 8.48 (d, J=8.7 Hz, 1 H), 8.10 (d, J=2.5 Hz, 1 H), 7.98 (dd, J=7.8, 1.8 Hz, 1 H), 7.91 (dd, J=8.9, 2.5 Hz, 1 H), 7.81 (dd, J=7.9, 1.7 Hz, 1 H), 7.32-7.43 (m, 5 H), 3.89 (s, 3 H), 3.82 (s, 3 H), 3.40 (s, 3 H). The corresponding methyl ester was hydrolyzed as described above to afford a white solid. ¹H NMR (400 MHz, DMSO-d₆) δ 12.02 (s, 1 H), 8.70 (d, J=9.1 Hz, 1 H), 8.14 (d, J=2.5 Hz, 1 H), 8.01 (dd, J=7.7, 1.2 Hz, 1 H), 7.90 (dd, J=9.0, 2.4 Hz, 1 H), 7.76 (dd, J=7.9, 1.5 Hz, 1 H), 7.11-7.44 (m, 5 H), 3.81 (s, 3 H), 3.39 (s, 3 H).

### 5-bromo-2-[(4-{[methyl(pyridin-2-yl)amino]sulfonyl}benzoyl)amino]benzoic acid

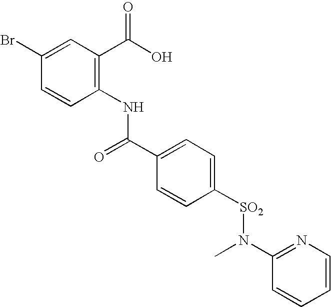

4-{[methyl(pyridin-2-yl)amino]sulfonyl}benzoic acid (292 mg, 1.0 mmol) was suspended in CH₂Cl₂ (10 mL) and (COCl)₂ added (725 mg, 0.5 mL, 5.7 mmol). A catalytic amount of DMF was then added and the mixture stirred for 4 hrs. The solvent was then removed in vacuo to give the acid chloride as an oil. The oil was dissolved in CHCl₃ (10 mL). Methyl 2-amino-5-bromobenzoate (230 mg, 1.0 mmol) was added followed by pyridine (1 mL). The solution was stirred at room temperature for an additional 12 hrs then poured into 1 M HCl (20 mL) and extracted with EtOAc (3×20 mL). The combined organic solutions were dried over Na₂SO₄ and concentrated in vacuo. The resulting residue was purified by silica gel chromatography (20% EtOAc in hexane) to provide 317 mg of the desired methyl ester (63%). The ester was treated with LiOH in 1:1:1 THF/MeOH/H₂O for 12 hrs followed by acidification and extraction with EtOAc. The organic solution was dried over Na₂SO₄ and then concentrated in vacuo. The title compound (281 mg, 91%, 57% overall) was obtained as a tan solid after recrystalization from MeOH. H NMR (400 MHz, DMSO) 3.72 (s, 3H), 7.28 (dd, 1H), 7.56 (d, 1 H), 7.81-7.91 (m, 4H), 8.07 (d, 2H), 8.12 (d, 1H), 8.32 (dd, 1H), 8.54 (d, 1H), 12.10 (s, 1H). C NMR (100 MHz, DMSO) 36.10, 101.83, 115.38, 120.21, 120.30, 122.15, 122.90, 128.33, 128.42, 133.62, 136.95, 138.77, 139.91, 140.30, 148.52, 153.13, 163.81, 168.81. MS (FAB) m/z (rel. intensity) 490 (MH⁺, 30), 492 (32), 490 (30), 414 (28), 413 (83), 109 (31), 107 (36), 95 (25), 91 (99), 57 (73), 55 (28). HRMS (FAB) calcd for C₂₀H₁₆BRN₃O₅S+H₁ 490.0073, found 490.0067.

### 5-bromo-2-{[3-(1H-indol-1-ylsulfonyl)benzoyl]amino}benzoic acid

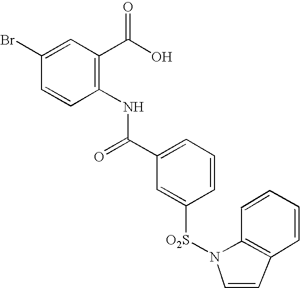

3-(1H-indol-1-ylsulfonyl)benzoic acid (301 mg, 1.0 mmol) was suspended in CH₂Cl₂ (10 mL) and (COCl)₂ added (725 mg, 0.5 mL, 5.7 mmol). A catalytic amount of DMF was then added and the mixture stirred for 4 hrs. The solvent was then removed in vacuo to give the acid chloride as an oil. The oil was dissolved in CHCl₃ (10 mL). Methyl 2-amino-5-bromobenzoate (230 mg, 1.0 mmol) was added followed by pyridine (1 mL). The solution was stirred at room temperature for an additional 12 hrs then poured into 1 M HCl (20 mL) and extracted with EtOAc (3×20 mL). The combined organic solutions were dried over Na₂SO₄ and concentrated in vacuo. The resulting residue was purified by silica gel chromatography (10% EtOAc in hexane) to provide 287 mg of the desired methyl ester (56%). The ester was treated with LiOH in 1:1:1 THF/MeOH/H₂O for 12 hrs followed by acidification and extraction with EtOAc. The organic solution was dried over Na₂SO₄ and then concentrated in vacuo. The title compound (53 mg, 11%, 6% overall) was obtained as a white solid after recrystalization from MeOH. H NMR (400 MHz, DMSO) 6.90 (d, 1H), 7.27 (t, 1H), 7.37 (t, 1H), 7.62 (d, 1H), 7.82 (t, 1H), 7.87-7.89 (m, 2H), 8.00 (d, 1H), 8.05 (d, 1H), 8.19-8.25 (m, 3H), 8.47 (s, 1H), 11.35 (s, 1H).

### 5-bromo-2-{[3-(2,3-dihydro-1H-indol-1-ylsulfonyl)benzoyl]amino}benzoic acid

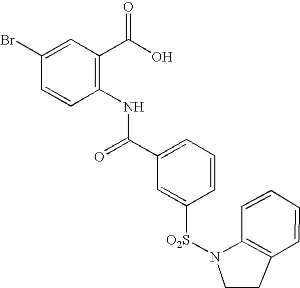

3-(2,3-dihydro-1H-indol-1-ylsulfonyl)benzoic acid (305 mg, 1.0 mmol) was suspended in CH₂Cl₂ (10 mL) and (COCl)₂ added (725 mg, 0.5 mL, 5.7 mmol). A catalytic amount of DMF was then added and the mixture stirred for 4 hrs. The solvent was then removed in vacuo to give the acid chloride as an oil. The oil was dissolved in CHCl₃ (10 mL). Methyl 2-amino-5-bromobenzoate (230 mg, 1.0 mmol) was added followed by pyridine (1 mL). The solution was stirred at room temperature for an additional 12 hrs then poured into 1 M HCl (20 mL) and extracted with EtOAc (3×20 mL). The combined organic solutions were dried over Na₂SO₄ and concentrated in vacuo. The resulting residue was purified by silica gel chromatography (10% EtOAc in hexane) to provide 381 mg of the desired methyl ester (74%). The ester was treated with LiOH in 1:1:1 THF/MeOH/H₂O for 12 hrs followed by acidification and extraction with EtOAc. The organic solution was dried over Na₂SO₄ and then concentrated in vacuo. The title compound (344 mg, 93%, 68% overall) was obtained as a white solid after recrystalization from MeOH. H NMR (400 MHz, DMSO) 2.94 (t, 2H), 4.00 (t, 2H), 6.99 (t, 1H), 7.15-7.23 (m, 2H), 7.52 (d, 1H), 7.80 (t, 1H), 7.89 (dd, 1H), 8.05-8.07 (m, 2H), 8.20 (d, 1 H), 8.28 (d, 1H), 8.35 (s, 1H), 11.40 (s, 1H).

### 5-bromo-2-{[4-(pyrrolidin-1-ylsulfonyl)benzoyl]amino}benzoic acid

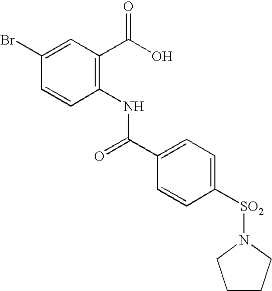

4-(pyrrolidin-1-ylsulfonyl)benzoic acid (255 mg, 1.0 mmol) was suspended in CH₂Cl₂ (10 mL) and (COCl)₂ added (725 mg, 0.5 mL, 5.7 mmol). A catalytic amount of DMF was then added and the mixture stirred for 4 hrs. The solvent was then removed in vacuo to give the acid chloride as an oil. The oil was dissolved in CHCl₃ (10 mL). Methyl 2-amino-5-bromobenzoate (230 mg, 1.0 mmol) was added followed by pyridine (1 mL). The solution was stirred at room temperature for an additional 12 hrs then poured into 1 M HCl (20 mL) and extracted with EtOAc (3×20 mL). The combined organic solutions were dried over Na₂SO₄ and concentrated in vacuo. The resulting residue was purified by silica gel chromatography (10% EtOAc in hexane) to provide 331 mg of the desired methyl ester (71%). The ester was treated with LiOH in 1:1:1 THF/MeOH/H₂O for 12 hrs followed by acidification and extraction with EtOAc. The organic solution was dried over Na₂SO₄ and then concentrated in vacuo. The title compound (308 mg, 96%, 68% overall) was obtained as a pale yellow solid after recrystalization from MeOH. H NMR (400 MHz, DMSO) 1.67 (m, 4H), 3.19 (m, 4H), 7.88 (dd, 1H), 8.02 (d, 2H), 8.12-8.16 (m, 3H), 8.58 (d, 1H), 12.10 (s, 1H).

### 5-cyano-2-{[4-(pyrrolidin-1-ylsulfonyl)benzoyl]amino}benzoic acid

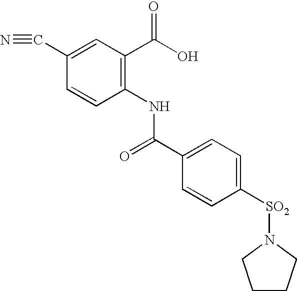

4-(pyrrolidin-1-ylsulfonyl)benzoic acid (255 mg, 1.0 mmol) was suspended in CH₂Cl₂ (10 mL) and (COCl)₂ added (725 mg, 0.5 mL, 5.7 mmol). A catalytic amount of DMF was then added and the mixture stirred for 4 hrs. The solvent was then removed in vacuo to give the acid chloride as an oil. The oil was dissolved in CHCl₃ (10 mL). Methyl 2-amino-5-cyanobenzoate (176 mg, 1.0 mmol) was added followed by pyridine (1 mL). The solution was stirred at room temperature for an additional 12 hrs then poured into 1 M HCl (20 mL) and extracted with EtOAc (3×20 mL). The combined organic solutions were dried over Na₂SO₄ and concentrated in vacuo. The resulting residue was purified by silica gel chromatography (10% EtOAc in hexane) to provide 293 mg of the desired methyl ester (71%). The ester was treated with LiOH in 1:1:1 THF/MeOH/H₂O for 12 hrs followed by acidification and extraction with EtOAc. The organic solution was dried over Na₂SO₄ and then concentrated in vacuo. The title compound (262 mg, 92%, 65% overall) was obtained as a pale yellow solid after recrystalization from MeOH. H NMR (400 MHz, DMSO) 1.67 (m, 4H), 3.20 (m, 4H), 8.04 (d, 2H), 8.11-8.18 (m, 3H), 8.42 (d, 1H), 8.80 (d, 1H), 12.25 (s, 1H),

### 5-bromo-2-{[3-(pyrrolidin-1-ylsulfonyl)benzoyl]amino}benzoic acid

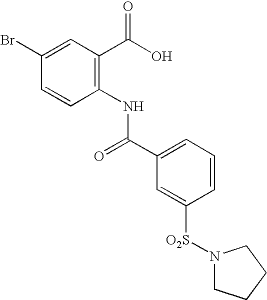

3-(pyrrolidin-1-ylsulfonyl)benzoic acid (255 mg, 1.0 mmol) was suspended in CH₂Cl₂ (10 mL) and (COCl)₂ added (725 mg, 0.5 mL, 5.7 mmol). A catalytic amount of DMF was then added and the mixture stirred for 4 hrs. The solvent was then removed in vacuo to give the acid chloride as an oil. The oil was dissolved in CHCl₃ (10 mL). Methyl 2-amino-5-bromobenzoate (230 mg, 1.0 mmol) was added followed by pyridine (1 mL). The solution was stirred at room temperature for an additional 12 hrs then poured into 1 M HCl (20 mL) and extracted with EtOAc (3×20 mL). The combined organic solutions were dried over Na₂SO₄ and concentrated in vacuo. The resulting residue was purified by silica gel chromatography (10% EtOAc in hexane) to provide 333 mg of the desired methyl ester (71%). The ester was treated with LiOH in 1:1:1 THF/MeOH/H₂O for 12 hrs followed by acidification and extraction with EtOAc. The organic solution was dried over Na₂SO₄ and then concentrated in vacuo. The title compound (309 mg, 96%, 68% overall) was obtained as a pale yellow solid after recrystalization from MeOH. H NMR (400 MHz, DMSO) 1.67 (m, 4H), 3.20 (m, 4H), 7.85-7.89 (m, 2H), 8.08 (d, 1H), 8.13 (d, 1H), 8.25 (d, 1H), 8.32 (s, 1H), 8.60 (d, 1H), 12.20 (s, 1H).

### 5-cyano-2-({3-[(2-methylpyrrolidin-1-yl)sulfonyl]benzoyl}amino)benzoic acid

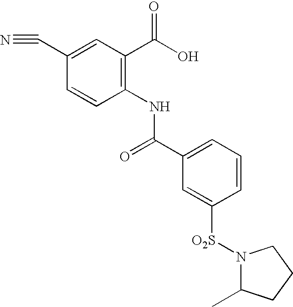

3-[(2-methylpyrrolidin-1-yl)sulfonyl]benzoic acid (269 mg, 1.0 mmol) was suspended in CH₂Cl₂ (10 mL) and (COCl)₂ added (725 mg, 5.7 mmol). A catalytic amount of DMF was then added and the mixture stirred for 4 hrs. The solvent was then removed in vacuo to give the acid chloride as an oil. The oil was dissolved in CHCl₃ (10 mL). Methyl 2-amino-5-cyanobenzoate (176 mg, 1.0 mmol) was added followed by pyridine (1 mL). The solution was stirred at room temperature for an additional 12 hrs then poured into 1 M HCl (20 mL) and extracted with EtOAc (3×20 mL). The combined organic solutions were dried over Na₂SO₄ and concentrated in vacuo. The resulting residue was purified by silica gel chromatography (10% EtOAc in hexane) to provide 350 mg of the desired methyl ester (82%). The ester was treated with LiOH in 1:1:1 THF/MeOH/H₂O for 12 hrs followed by acidification and extraction with EtOAc. The organic solution was dried over Na₂SO₄ and then concentrated in vacuo. The title compound (308 mg, 91%, 75% overall) was obtained as a white solid after recrystalization from MeOH. H NMR (400 MHz, DMSO) 1.25 (d, 3H), 1.41-1.47 (m, 2H), 1.59-1.67 (m, 1H), 1.77-1.83 (m, 1H), 3.12-3.18 (m, 1H), 3.36-3.42 (m, 1H), 3.69 (m, 1H), 7.88 (t, 1H), 8.12 (d, 1H), 8.13 (d, 1H), 8.25 (d, 1H), 8.34 (s, 1H), 8.42 (d, 1H), 8.83 (d, 1H), 12.55 (s, 1H).

### 5-cyano-2-({3-[(2,5-dimethylpyrrolidin-1-yl)sulfonyl]benzoyl}amino)benzoic acid

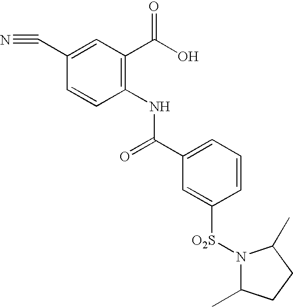

3-[(2,5-dimethylpyrrolidin-1-yl)sulfonyl]benzoic acid (283 mg, 1.0 mmol) was suspended in CH₂Cl₂ (10 mL) and (COCl)₂ added (725 mg, 5.7 mmol). A catalytic amount of DMF was then added and the mixture stirred for 4 hrs. The solvent was then removed in vacuo to give the acid chloride as an oil. The oil was dissolved in CHCl₃ (10 mL). Methyl 2-amino-5-cyanobenzoate (176 mg, 1.0 mmol) was added followed by pyridine (1 mL). The solution was stirred at room temperature for an additional 12 hrs then poured into 1 M HCl (20 mL) and extracted with EtOAc (3×20 mL). The combined organic solutions were dried over Na₂SO₄ and concentrated in vacuo. The resulting residue was purified by silica gel chromatography (10% EtOAc in hexane) to provide 293 mg of the desired methyl ester (66%). The ester was treated with LiOH in 1:1:1 THF/MeOH/H₂O for 12 hrs followed by acidification and extraction with EtOAc. The organic solution was dried over Na₂SO₄ and then concentrated in vacuo. The title compound (273 mg, 97%, 64% overall) was obtained as a tan solid after recrystalization from MeOH. H NMR (400 MHz, DMSO) 1.29 (d, 6H), 1.49 (m, 4H), 3.67 (m, 2H), 7.88 (t, 1H), 8.12 (d, 1H), 8.13 (d, 1H), 8.25 (d, 1H), 8.34 (s, 1H), 8.42 (d, 1H), 8.83 (d, 1H), 12.55 (s, 1H).

### 5-cyano-2-{[3-(pyrrolidin-1-ylsulfonyl)benzoyl]amino}benzoic acid was produced from methyl 2-{[3-(chlorosulfonyl)benzoyl]amino}-5-cyanobenzoate. H NMR (300 MHz, DMSO) 1.67 (m, 4H), 3.20 (m, 4H), 7.88 (t, 1H), 8.09-8.14 (m, 2H), 8.26 (d, 1H), 8.33 (s, 1H), 8.42 (d, 1H), 8.83 (d, 1H), 12.56 (s, 1H)

### 5-chloro-2-{[3-(morpholin-4-yIsulfonyl)benzoyl]amino}benzoic acid

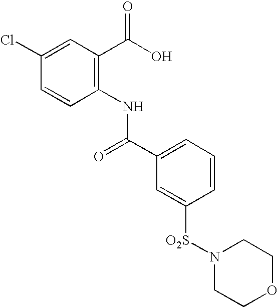

3-(morpholin-4-ylsulfonyl)benzoic acid (271 mg, 1.0 mmol) was suspended in CH₂Cl₂ (10 mL) and (COCl)₂ added (725 mg, 5.7 mmol). A catalytic amount of DMF was then added and the mixture stirred for 4 hrs. The solvent was then removed in vacuo to give the acid chloride as an oil. The oil was dissolved in CHCl₃ (10 mL). Methyl 2-amino-5-chlorobenzoate (185 mg, 1.0 mmol) was added followed by pyridine (1 mL). The solution was stirred at room temperature for an additional 12 hrs then poured into 1 M HCl (20 mL) and extracted with EtOAc (3×20 mL). The combined organic solutions were dried over Na₂SO₄ and concentrated in vacuo. The resulting residue was purified by silica gel chromatography (20% EtOAc in hexane) to provide 382 mg of the desired methyl ester (87%). The ester was treated with LiOH in 1:1:1 THF/MeOH/H₂O for 12 hrs followed by acidification and extraction with EtOAc. The organic solution was dried over Na₂SO₄ and then concentrated in vacuo. The title compound (351 mg, 95%, 83% overall) was obtained as a white solid after recrystalization from MeOH. H NMR (400 MHz, DMSO) 2.93 (m, 4H), 3.65 (m, 4H), 7.77 (dd, 1H), 7.91 (t, 1H), 7.99-8.02 (m, 2H), 8.25-8.29 (m, 2H), 8.65 (d, 1H), 12.17 (s, 1H).

5-bromo-2-{[3-(morpholin-4-ylsulfonyl)benzoyl]amino}benzoic acid and 2-{[3-(morpholin-4-ylsulfonyl)benzoyl]amino}-5-nitrobenzoic acid were produced in a similar fashion utilizing appropriate starting materials.

### 5-fluoro-2-{[3-(morpholin-4-ylsulfonyl)benzoyl]amino}benzoic acid

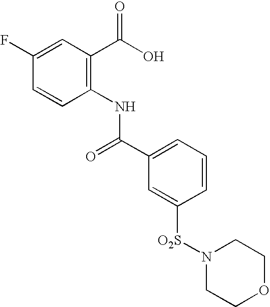

3-(morpholin-4-ylsulfonyl)benzoic acid (271 mg, 1.0 mmol) was suspended in CH₂Cl₂ (10 mL) and (COCl)₂ added (725 mg, 5.7 mmol). A catalytic amount of DMF was then added and the mixture stirred for 4 hrs. The solvent was then removed in vacuo to give the acid chloride as an oil. The oil was dissolved in CHCl₃ (10 mL). Methyl 2-amino-5-fluorobenzoate (170 mg, 1.0 mmol) was added followed by pyridine (1 mL). The solution was stirred at room temperature for an additional 12 hrs then poured into 1 M HCl (20 mL) and extracted with EtOAc (3×20 mL). The combined organic solutions were dried over Na₂SO₄ and concentrated in vacuo. The resulting residue was purified by silica gel chromatography (20% EtOAc in hexane) to provide 367 mg of the desired methyl ester (87%). The ester was treated with LiOH in 1:1:1 THF/MeOH/H₂O for 12 hrs followed by acidification and extraction with EtOAc. The organic solution was dried over Na₂SO₄ and then concentrated in vacuo. The title compound (328 mg, 92%, 80% overall) was obtained as a white solid after recrystalization from MeOH. H NMR (400 MHz, DMSO) 2.93 (m, 4H), 3.65 (m, 4H), 7.58 (m, 1H), 7.77 (dd, 1H), 7.90 (t, 1H), 8.00 (d, 1H), 8.26-8.29 (m, 2H), 8.60 (dd, 1H), 12.02 (s, 1H).

### 5-cyano-2-{[3-(piperidin-1-ylsulfonyl)benzoyl]amino}benzoic acid

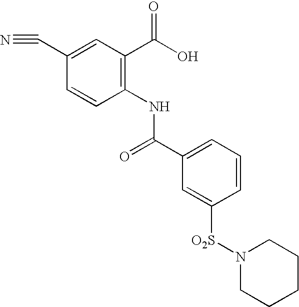

3-(piperidin-1-ylsulfonyl)benzoic acid (269 mg, 1.0 mmol) was suspended in CH₂Cl₂ (10 mL) and (COCl)₂ added (725 mg, 5.7 mmol). A catalytic amount of DMF was then added and the mixture stirred for 4 hrs. The solvent was then removed in vacuo to give the acid chloride as an oil. The oil was dissolved in CHCl₃ (10 mL). Methyl 2-amino-5-cyanobenzoate (176 mg, 1.0 mmol) was added followed by pyridine (1 mL). The solution was stirred at room temperature for an additional 12 hrs then poured into 1 M HCl (20 mL) and extracted with EtOAc (3×20 mL). The combined organic solutions were dried over Na₂SO₄ and concentrated in vacuo. The resulting residue was purified by silica gel chromatography (10% EtOAc in hexane) to provide 307 mg of the desired methyl ester (72%). The ester was treated with LiOH in 1:1:1 THF/MeOH/H₂O for 12 hrs followed by acidification and extraction with EtOAc. The organic solution was dried over Na₂SO₄ and then concentrated in vacuo. The title compound (279 mg, 94%) was obtained as a white solid after recrystalization from MeOH. H NMR (400 MHz, DMSO) 1.37 (m, 2H), 1.56 (m, 4H), 2.95 (m, 4H), 7.90 (t, 1H), 8.02 (d, 1H), 8.13 (dd, 1H), 8.27 (m, 2H), 8.42 (d, 1H), 8.83 (1H), 12.55 (s, 1H).

### 5-cyano-2-{[3-(1H-indol-1-ylsulfonyl)benzoyl]amino}benzoic acid

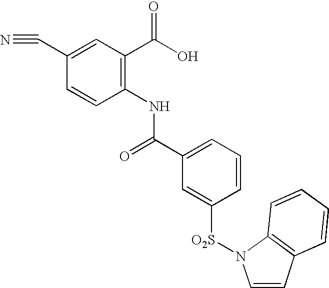

Indole (150 mg, 1.25 mmol) was dissolved in 15 ml of THF. NaH (100 mg, 60% disp. in oil, 2.5 mmol) was added and resulting suspension stirrted for 1 h. Methyl 2-{[3-(chlorosulfonyl)benzoyl]amino}-5-cyanobenzoate (378 mg, 1.0 mmol) was then added and the reaction stirred at room temperature of 12 hr. The mixture was poured into 1 M HCl (20 mL) and extracted with EtOAc (3×20 mL). The combined organic solutions were dried over Na₂SO₄ and concentrated in vacuo. The resulting residue was purified by silica gel chromatography, providing 252 mg (55%) of the desired methyl ester. The ester was treated with LiOH in 1:1:1 THF/MeOH/H₂O for 12 hrs followed by acidification and extraction with EtOAc. The organic solution was dried over Na₂SO₄ and then concentrated in vacuo. The title compound (24 mg, 10%) was obtained as a tan solid after recrystalization from MeOH. H NMR (400 MHz, DMSO) 6.89 (d, 1H), 7.28 (t, 1H), 7.37 (t, 1H), 7.61 (d, 1H), 7.81-7.86 (m, 2H), 8.01 (d, 1H), 8.11 (dd, 1H), 8.24 (t, 2H), 8.42 (d,iH), 8.52 (t, 1H), 8.75 (d, 1H).

### 5-cyano-2-({3-[(5-methoxy-1H-indol-1-yl)sulfonyl]benzoyl}amino)benzoic acid

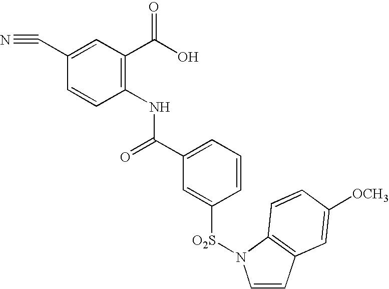

5-Methoxyindole (190 mg, 1.25 mmol) was dissolved in 15 ml of THF. NaH (100 mg, 60% disp. in oil, 2.5 mmol) was added and resulting suspension stirrted for 1 h. Methyl 2-{[3-(chlorosulfonyl)benzoyl]amino}-5-cyanobenzoate (378 mg, 1.0 mmol) was then added and the reaction stirred at room temperature of 12 hr. The mixture was poured into 1 M HCl (20 mL) and extracted with EtOAc (3×20 mL). The combined organic solutions were dried over Na₂SO₄ and concentrated in vacuo. The resulting residue was purified by silica gel chromatography, providing 236 mg (48%) of the desired methyl ester. The ester was treated with LiOH in 1:1:1 THF/MeOH/H₂O for 12 hrs followed by acidification and extraction with EtOAc. The organic solution was dried over Na₂SO₄ and then concentrated in vacuo. The title compound (31 mg, 13%) was obtained as a white solid after recrystalization from MeOH. H NMR (400 MHz, DMSO) 3.73 (s, 3H), 6.81 (d, 1H), 6.97 (dd, 1H), 7.11 (d, 1H), 7.79 (d, 1H), 7.82 (t, 1H), 7.89 (d, 1H), 8.11 (dd, 1H), 8.21 (t, 1H), 8.42 (d, 1H), 8.48 (t, 1H), 8.76 (d, 1H).

5-cyano-2-({3-[(7-methoxy-1H-indol-1-yl)sulfonyl]benzoyl}amino)benzoic acid was produced using 7-Methoxyindole. H NMR (400 MHz, DMSO) 3.84 (s, 3H), 6.79 (d, 1H), 6.83 (d, 1H), 7.29 (t, 1H), 7.61 (d, 1H), 7.75 (d, 1H), 7.80 (m, 2H), 8.17 (d, 1H), 8.28 (d, 1H), 8.32 (d, 1H), 8.56 (t, 1H), 8.73 (d, 1H).

5-cyano-2-({3-[(6-methoxy-1H-indol-1-yl)sulfonyl]benzoyl}amino)benzoic acid was produced using 6-Methoxyindole. H NMR (300 MHz, DMSO) 3.85 (s, 3H), 6.78 (d, 1H), 6.89 (dd, 1H), 7.47-7.49 (m, 2H), 7.71 (d, 1H), 7.79-7.85 (m, 2H), 8.20 (d, 1H), 8.29 (d, 1H), 8.34 (d, 1H), 8.59 (t, 1H), 8.75 (d, 1H).

### 2-({3-[(5-chloro-1H-indol-1-yl)sulfonyl]benzoyl}amino)-5-cyanobenzoic acid

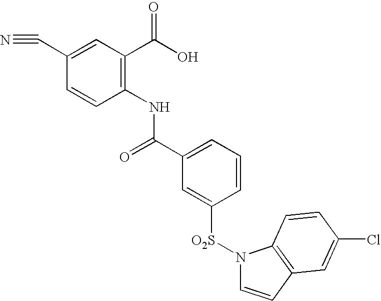

5-Chloroindole (190 mg, 1.25 mmol) was dissolved in 15 ml of THF. NaH (100 mg, 60% disp. in oil, 2.5 mmol) was added and resulting suspension stirrted for 1 h. Methyl 2-{[3-(chlorosulfonyl)benzoyl]amino}-5-cyanobenzoate (378 mg, 1.0 mmol) was then added and the reaction stirred at room temperature of 12 hr. The mixture was poured into 1 M HCl (20 mL) and extracted with EtOAc (3×20 mL). The combined organic solutions were dried over Na₂SO₄ and concentrated in vacuo. The resulting residue was purified by silica gel chromatography, providing 311 mg (63%) of the desired methyl ester. The ester was treated with LiOH in 1:1:1 THF/MeOH/H₂O for 12 hrs followed by acidification and extraction with EtOAc. The organic solution was dried over Na₂SO₄ and then concentrated in vacuo. The title compound (27 mg, 9%) was obtained as a white solid after recrystalization from MeOH. H NMR (400 MHz, DMSO) 6.88 (d, 1H), 7.42 (dd, 1H), 7.71 (d, 1H), 7.85 (t, 1H), 7.94 (d, 1H), 8.02 (d, 1H), 8.12 (dd, 1H), 8.25 (m, 2H), 8.43 (d, 1H), 8.52 (t, 1H), 8.76 (d, 1H).

5-cyano-2-({3-[(5-fluoro-1H-indol-1-yl)sulfonyl]benzoyl}amino)benzoic acid was produced utilizing 5-Fluoroindole. H NMR (400 MHz, DMSO) 6.89 (d, 1 H), 7.23 (dt, 1H), 7.44 (dd, 1H), 7.85 (t, 1H), 7.96 (d, 1H), 8.00 (dd, 1H), 8.13 (dd, 1H), 8.22 (d, 1H), 8.27 (d, 1H), 8.37 (d, 1H), 8.51 (m, 2H).

### 5-cyano-2-{[3-({methyl[(1R)-1-phenylethyl]amino}sulfonyl)benzoyl]amino}benzoic acid

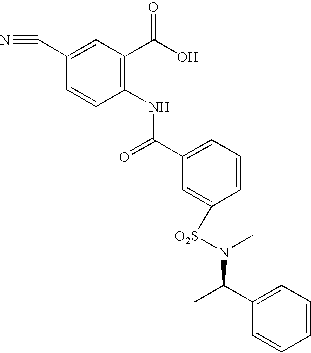

Methyl 2-{[3-(chlorosulfonyl)benzoyl]amino}-5-cyanobenzoate (378 mg, 1.0 mmol) was dissolved in 15 mL of CHCl₃. N-methyl-N—[(1R)-1-phenylethyl]amine (270 mg, 2.0 mmol) and Et₃N (1 mL) were then added and the reaction stirred at room temperature for 12 hr. The mixture was poured into 1 M HCl (20 mL) and extracted with EtOAc (3×20 mL). The combined organic solutions were dried over Na₂SO₄ and concentrated in vacuo. The resulting residue was purified by silica gel chromatography, providing 400 mg (84%) of the desired methyl ester. The ester was treated with LiOH in 1:1:1 THF/MeOH/H₂O for 12 hrs followed by acidification and extraction with EtOAc. The organic solution was dried over Na₂SO₄ and then concentrated in vacuo. The title compound (300 mg, 77%) was obtained as a white solid after recrystalization from MeOH. H NMR (300 MHz, DMSO) 1.23 (d, 3H), 2.61 (s, 3H), 5.22 (q, 1H), 7.26-7.35 (m, 5H), 7.87 (t, 1H), 8.12 (d, 1H), 8.13 (d, 1H), 8.25 (d, 1H), 8.36 (s, 1H), 8.42 (d, 1H), 8.83 (d, 1H), 12.55 (s, 1H).

5-cyano-2-{[3-({methyl[(1S)-1-phenylethyl]amino}sulfonyl)benzoyl]amino}benzoic acid was produced from N-methyl-N-[(1S)-1-phenylethyl]amine. H NMR (300 MHz, DMSO) 1.23 (d, 3H), 2.61 (s, 3H), 5.22 (q, 1H), 7.26-7.35 (m, 5H), 7.87 (t, 1I), 8.12 (d, 1H), 8.13 (d, 1H), 8.25 (d, 1H), 8.36 (s, 1H), 8.42 (d, 1H), 8.83 (d, 1H), 12.55 (s, 1H).

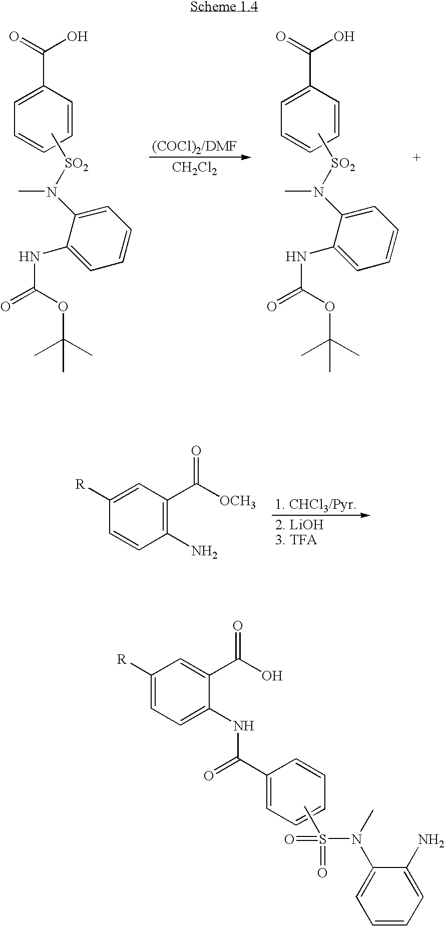

### 2-[(4-{[(2-aminophenyl)(methyl)amino]sulfonyl}benzoyl)amino]-5-bromobenzoic acid

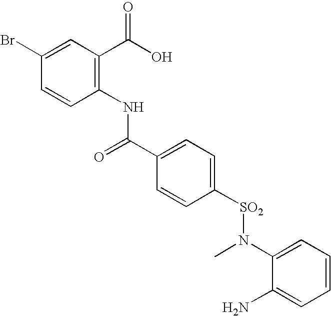

4-{[{2-[(tert-butoxycarbonyl)amino]phenyl}(methyl)amino]sulfonyl}benzoic (406 mg, 1.0 mmol) was suspended in CH₂Cl₂ (10 mL) and (COCl)₂ added (725 mg, 5.7 mmol). A catalytic amount of DMF was then added and the mixture stirred for 4 hrs. The solvent was then removed in vacuo to give the acid chloride as an oil. The oil was dissolved in CHCl₃ (10 mL). Methyl 2-amino-5-bromobenzoate (230 mg, 1.0 mmol) was added followed by pyridine (1 mL). The solution was stirred at room temperature for an additional 12 hrs then poured into 1 M HCl (20 mL) and extracted with EtOAc (3×20 mL). The combined organic solutions were dried over Na₂SO₄ and concentrated in vacuo. The resulting residue was purified by silica gel chromatography to provide 346 mg of the desired methyl ester (56%). The ester was treated with LiOH in 1:1:1 THF/MeOH/H₂O for 12 hrs followed by acidification. The resulting solid was dried in the air then dissolved in CH₂Cl₂/TFA and stirred for 10 additional hours. The solvent was removed in vacuo and the remaining solid was recrystalized from MeOH to give the title compound (163 mg, 58%) as a white solid. H NMR (400 MHz, DMSO) 3.12 (s, 3H), 6.36-6.43 (m, 2H), 6.78 (d, 1H), 6.99-7.04 (m, 1H), 7.83-7.93 (m, 3H), 8.13-8.16 (m, 2H), 8.28-8.29 (m, 1H), 8.60 (t, 1H (s, 1H).

### 2-[(3-{[(2-aminophenyl)(methyl)amino]sulfonyl}benzoyl)amino]-5-cyanobenzoic acid

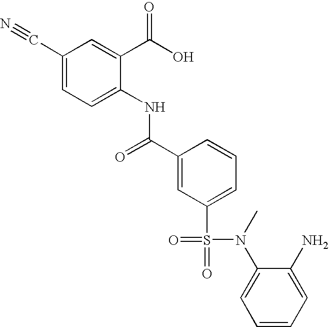

3-{[{2-[(tert-butoxycarbonyl)amino]phenyl}(methyl)amino]sulfonyl}benzoic (406 mg, 1.0 mmol) was suspended in CH₂Cl₂ (10 mL) and (COCl)₂ added (725 mg, 5.7 mmol). A catalytic amount of DMF was then added and the mixture stirred for 4 hrs. The solvent was then removed in vacuo to give the acid chloride as an oil. The oil was dissolved in CHCl₃ (10 mL). Methyl 2-amino-5-cyanobenzoate (176 mg, 1.0 mmol) was added followed by pyridine (1 mL). The solution was stirred at room temperature for an additional 12 hrs then poured into 1 M HCl (20 mL) and extracted with EtOAc (3×20 mL). The combined organic solutions were dried over Na₂SO₄ and concentrated in vacuo. The resulting residue was purified by silica gel chromatography to provide 344 mg of the desired methyl ester (61%). The ester was treated with LiOH in 1:1:1 THF/MeOH/H₂O for 12 hrs followed by acidification. The resulting solid was dried in the air then dissolved in CH₂Cl₂/TFA and stirred for 10 additional hours. The solvent was removed in vacuo and the remaining solid was recrystalized from MeOH to give the title compound (34 mg, 12%) as a white solid. H NMR (400 MHz, DMSO) 3.11 (s, 1H), 6.37 (m, 2H), 6.76 (d, 1H), 7.00 (m, 1H), 7.84-7.93 (m, 2H), 8.31 (dd, 1H), 8.31 (m, 2H), 8.42 (d, 1H), 8.83 (d, 1H), 12.55 9s, 1H).

### Preparation of Methyl 2-amino-5-formylbenzoate

To a solution of methyl anthranilate (7.75 g, 51.3 mmol, Aldrich) in DMF (50 mL) was added NIS (11.5 g, 51.3 mmol, Aldrich). The solution was stirred for 63 hours before being added to a separatory funnel with 200 mL of MTBE and washed with 5×200 mL of water. The organics were dried over MgSO₄ and evaporated yielding 13.8 g of tan solid as methyl 2-amino-5-iodobenzoate. A mixture of methyl 2-amino-5-iodobenzoate (3.13 g, 11.3 mmol) and tetrakis(triphenylphosphine)palladium(0) (282 mg, 0.244 mmol, Strem) was placed under 1 atm of CO. THF (20 mL) was added, and the solution was heated to 60° C. Tri-n-butyltin hydride (3.7 mL, 12.7 mmol, Aldrich) was added dropwise with rapid stirring over 4 hours. The dark orange solution was heated a further 45 minutes and then added to a separatory funnel with 150 mL of EtOAc. This solution was washed with 2×150 mL of saturated aqueous NaHCO₃ followed by 100 mL of brine. It was dried over MgSO₄ and evaporated leaving a brown oil that was purified by chromatography using a Biotage Flash 40 M silica cartridge with a gradient from CH₂Cl₂ to 5% EtOAc in CH₂Cl₂ as eluent. This chromatography failed to remove all of the tin, so the product was re-chromatographed using a Biotage Flash 40 M silica cartridge with 5% EtOAc in CH₂Cl₂ as eluent. Yield was 863 mg of white solid.

### 5-Formyl-2-{[3-(morpholin-4-ylsulfonyl)benzoyl]amino}benzoic acid

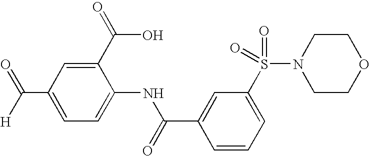

To 3-(morpholin-4-ylsulfonyl)benzoic acid (1.12 g, 4.13 mmol) in CH₂Cl₂ (60 mL) was added DMF (20 μL) and oxalyl chloride (450 μL, 5.16 mmol). The mixture was stirred for 3.75 hours, and the solvent and excess oxalyl chloride were removed by rotary evaporation. The residue was dissolved in CH₂Cl₂ (20 mL), and methyl 2-amino-5-formylbenzoate (637 mg, 3.56 mmol) in pyridine (8 mL) was added. The mixture was stirred overnight and then added to a separatory funnel with 100 mL of CH₂Cl₂. This solution was washed with 2×100 mL of 1 M aqueous HCl and 100 mL of brine. The CH₂Cl₂ was evaporated in the presence of silica gel, and the product was purified by chromatography using a Biotage Flash 40 M silica cartridge with a gradient from 5% EtOAc in CH₂Cl₂ to 10% EtOAc in CH₂Cl₂ as eluent. Yield was 636 mg of yellow solid as the methyl ester. To a mixture of the corresponding methyl ester (318 mg, 0.735 mmol) in dioxane (15 mL) was added 1 M aqueous sodium hydroxide (1.5 mL). The mixture was stirred at room temperature for 2 hours. The reaction mixture was added to a separatory funnel with 100 mL of 1 M aqueous HCl, and the product was extracted into 100 mL of EtOAc. The EtOAc was washed with an additional 100 mL of 1 M aqueous HCl followed by 100 mL of water. It was then dried over MgSO₄ and evaporated. The residue was recrystallized from hot ethanol. The solids were washed with ethanol followed by heptane and then dried at 100° C. under vacuum yielding 64 mg of tan solid. ¹H NMR (400 MHz, DMSO-d₆) δ 12.65 (s, 1 H), 10.00 (s, 1 H), 8.89 (d, J=8.7 Hz, 1 H), 8.60 (d, J=2.1 Hz, 1 H), 8.29-8.33 (m, 2 H), 8.20 (dd, J=8.7, 2.1 Hz, 1 H), 8.03 (d, J=8.1 Hz, 1 H), 7.93 (t, J=7.8 Hz, 1 H), 3.63-3.68 (m, 4 H), 2.92-2.97 (m, 4 H).

### 5-bromo-2-{[3-(morpholin-4-ylsulfonyl)benzoyl]amino}benzoic acid

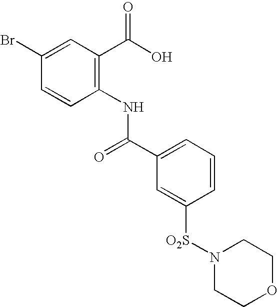

3-(morpholin-4-ylsulfonyl)benzoic acid (271 mg, 1.0 mmol) was suspended in CH₂Cl₂ (10 mL) and (COCl)₂ added (725 mg, 5.7 mmol). A catalytic amount of DMF was then added and the mixture stirred for 4 hrs. The solvent was then removed in vacuo to give the acid chloride as an oil. The oil was dissolved in CHCl₃ (10 mL). Methyl 2-amino-5-bromobenzoate (230 mg, 1.0 mmol) was added followed by pyridine (1 mL). The solution was stirred at room temperature for an additional 12 hrs then poured into 1 M HCl (20 mL) and extracted with EtOAc (3×20 mL). The combined organic solutions were dried over Na₂SO₄ and concentrated in vacuo. The resulting residue was purified by silica gel chromatography (20% EtOAc in hexane) to provide 367 mg of the desired methyl ester (76%). The ester was treated with LiOH in 1:1:1 THF/MeOH/H₂O for 12 hrs followed by acidification and extraction with EtOAc. The organic solution was dried over Na₂SO₄ and then concentrated in vacuo. The title compound (328 mg, 92%, 70% overall) was obtained as a white solid after recrystalization from MeOH. H NMR (400 MHz, DMSO) 2.93 (m, 4H), 3.65 (m, 4H), 7.88 (dd, 1H), 7.90 (d, 1H), 8.00 (d, 1H), 8.13 (d, 1H), 8.25-8.29 (m, 2H), 8.59 (d, 1H), 12.21 (s, 1H).

### 2-{[3-(morpholin-4-ylsulfonyl)benzoyl]amino}-5-nitrobenzoic acid

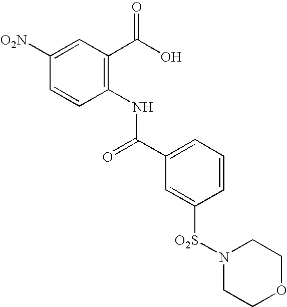

3-(morpholin-4-ylsulfonyl)benzoic acid (271 mg, 1.0 mmol) was suspended in CH₂Cl₂ (10 mL) and (COCl)₂ added (725 mg, 5.7 mmol). A catalytic amount of DMF was then added and the mixture stirred for 4 hrs. The solvent was then removed in vacuo to give the acid chloride as an oil. The oil was dissolved in CHCl₃ (10 mL). Methyl 2-amino-5-nitrobenzoate (196 mg, 1.0 mmol) was added followed by pyridine (1 mL). The solution was stirred at room temperature for an additional 12 hrs then poured into 1 M HCl (20 mL) and extracted with EtOAc (3×20 mL). The combined organic solutions were dried over Na₂SO₄ and concentrated in vacuo. The resulting residue was purified by silica gel chromatography (20% EtOAc in hexane) to provide 108 mg of the desired methyl ester (24%). The ester was treated with LiOH in 1:1:1 THF/MeOH/H₂O for 12 hrs followed by acidification and extraction with EtOAc. The organic solution was dried over Na₂SO₄ and then concentrated in vacuo. The title compound (70 mg, 67%, 16% overall) was obtained as a yellow solid after recrystalization from MeOH. H NMR (400 MHz, DMSO) 2.94 (m, 4H), 3.65 (m, 4H), 7.94 (t, 1H), 8.04 (d, 1H), 8.29-8.33 (m, 2H), 8.55 (dd, 1H), 8.80 (d, 1H), 8.91 (d, 1H), 12.76 (s, 1H)

Methyl 2-({3-[(5-chloro-2,3-dihydro-1H-indol-1-yl)sulfonyl]benzoyl}amino)-5-formylbenzoate was prepared as described above using 3-[(5-chloro-2,3-dihydro-1H-indol-1-yl)sulfonyl]benzoic acid. 2-({3-[(5-Chloro-2,3-dihydro-1H-indol-1-yl)sulfonyl]benzoyl}amino)-5-formylbenzoic acid was prepared by hydrolizing the corresponding methyl ester. ¹H NMR (400 MHz, DMSO-d₆) δ 12.69 (s, 1 H), 10.00 (s, 1 H), 8.87 (d, J=8.7 Hz, 1 H), 8.61 (d, J=2.1 Hz, 1 H), 8.40 (s, 1 H), 8.27 (d, J=7.9 Hz, 1 H), 8.19 (dd, J=8.7, 2.1 Hz, 1 H), 8.09 (d, J=8.5 Hz, 1 H), 7.84 (t, J=7.8 Hz, 1 H), 7.53 (d, J=8.5 Hz, 1 H), 7.24-7.29 (m, 2 H), 4.02 (t, J=8.5 Hz, 2 H), 2.95 (t, J=8.4 Hz, 2 H).

### 2-({3-[(5-Chloro-2,3-dihydro-1H-indol-1-yl)sulfonyl]benzoyl}amino)-5-[(E)-(methoxyimino)methyl]benzoic acid

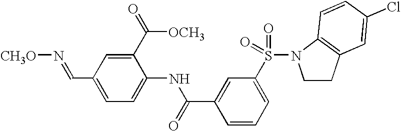

A slurry of methyl 2-({3-[(5-chloro-2,3-dihydro-1H-indol-1-yl)sulfonyl]benzoyl}amino)-5-formylbenzoate (475 mg, 0.952 mmol) and O-methylhydroxylamine hydrochloride (526 mg, 6.30 mmol, Aldrich) in 1:1 ethanol/pyridine (25 mL) was stirred for 2 days. The mixture was then added to a separatory funnel with 120 mL of CH₂Cl₂. This solution was washed with 2×100 mL of 1 M aqueous HCl followed by 100 mL of brine. The CH₂Cl₂ was evaporated in the presence of silica gel, and the product was purified by chromatography using a Biotage Flash 40 M silica cartridge with a gradient from CH₂Cl₂ to 2% EtOAc in CH₂Cl₂ as eluent. Yield was 411 mg of white solid as the methyl ester. To a mixture of the corresponding methyl ester (288 mg, 0.545 mmol) in dioxane (20 mL) was added 1 M aqueous sodium hydroxide (1.5 mL). The mixture was stirred at room temperature for 4.5 hours and then in a 50° C. oil bath for 30 minutes. The reaction mixture was added to a separatory funnel with 100 mL of 1 M aqueous HCl, and the product was extracted into 100 mL of EtOAc. The EtOAc was washed with an additional 100 mL of 1 M aqueous HCl followed by 100 mL of water. It was then dried over MgSO₄ and evaporated. The residue was recrystallized from hot ethanol/THF. The solids were washed with ethanol followed by heptane and then dried at 100° C. under vacuum yielding 127 mg of white solid. ¹H NMR (400 MHz, DMSO-d₆) δ 12.40 (s, 1 H), 8.70 (d, J=8.7 Hz, 1 H), 8.38 (s, 1 H), 8.31 (d, J=2.1 Hz, 1 H), 8.30 (s, 1 H), 8.25 (d, J=7.9 Hz, 1 H), 8.07 (d, J=8.1 Hz, 1 H), 7.91 (dd, J=8.7, 2.1 Hz, 1 H), 7.83 (t, J=7.9 Hz, 1 H), 7.52 (d, J=8.5 Hz, 1 H), 7.24-7.29 (m, 2 H), 4.02 (t, J=8.5 Hz, 2 H), 3.91 (s, 3 H), 2.95 (t, J=8.4 Hz, 2 H).

### 2-({3-[(5-Chloro-2,3-dihydro-1H-indol-1-yl)sulfonyl]benzoyl}amino)-5-[(E)-(hydroxyimino)methyl]benzoic acid

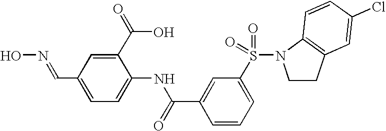

A slurry of methyl 2-({3-[(5-chloro-2,3-dihydro-1H-indol-1-yl)sulfonyl]benzoyl}amino)-5-formylbenzoate (627 mg, 1.26 mmol) and hydroxylamine hydrochloride (656 mg, 9.44 mmol, Mallinckrodt) in 1:1 ethanol/pyridine (25 mL) was stirred for 2 days. The mixture was then added to a separatory funnel with 120 mL of CH₂Cl₂. This solution was washed with 2×100 mL of 1 M aqueous HCl followed by 100 mL of brine. The CH₂Cl₂ was evaporated in the presence of silica gel, and the product was purified by chromatography using a Biotage Flash 40 M silica cartridge with 5% EtOAc in CH₂Cl₂ as eluent. Yield was 478 mg of white solid as the methyl ester. To a mixture of the corresponding methyl ester (363 mg, 0.706 mmol) in dioxane (20 mL) was added 1 M aqueous sodium hydroxide (1.5 mL). The mixture was stirred at room temperature for 4.5 hours. The reaction mixture was added to a separatory funnel with 100 mL of 1 M aqueous HCl, and the product was extracted into 100 mL of EtOAc. The EtOAc was washed with an additional 100 mL of 1 M aqueous HCl followed by 100 mL of water. It was then dried over MgSO₄ and evaporated. The residue was recrystallized from hot ethanol/THF. The solids were washed with ethanol followed by heptane and then dried at 100° C. under vacuum yielding 280 mg of white solid. Because NMR and CHN analysis were consistent with this material containing residual solvent, 200 mg of the material was heated in 50 mL of methanol. Solvent was removed, and the residue was again dried at 100° C. under vacuum yielding 183 mg of white solid. ¹H NMR (400 MHz, DMSO-d₆) δ 12.37 (s, 1 H), 11.31 (s, 1 H), 8.68 (d, J=8.7 Hz, 1 H), 8.38 (s, 1 H), 8.29 (d, J=1.9 Hz, 1 H), 8.25 (d, J=7.9 Hz, 1 H), 8.20 (s, 1 H), 8.07 (d, J=8.1 Hz, 1 H), 7.90 (dd, J=8.8, 2.0 Hz, 1 H), 7.83 (t, J=7.9 Hz, 1 H), 7.53 (d, J=8.5 Hz, 1 H), 7.24-7.29 (m, 2 H), 4.01 (t, 8.5, 2 H), 2.95 (t, J=8.4 Hz, 2 H).

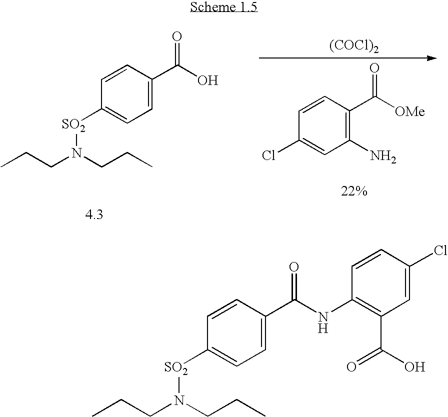

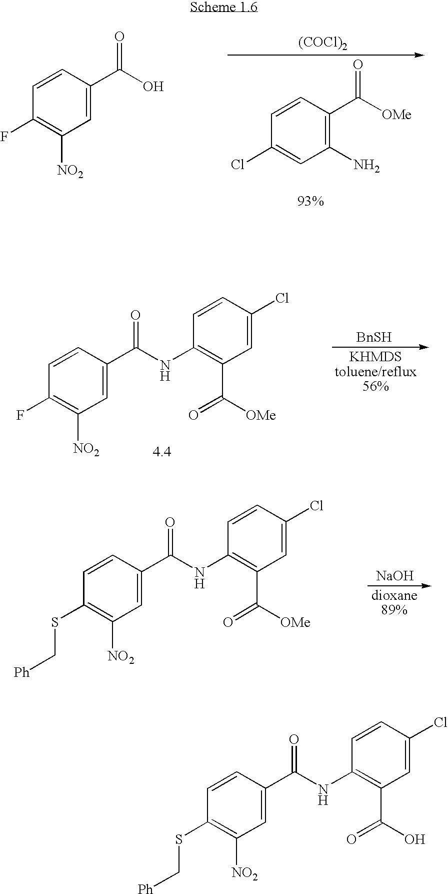

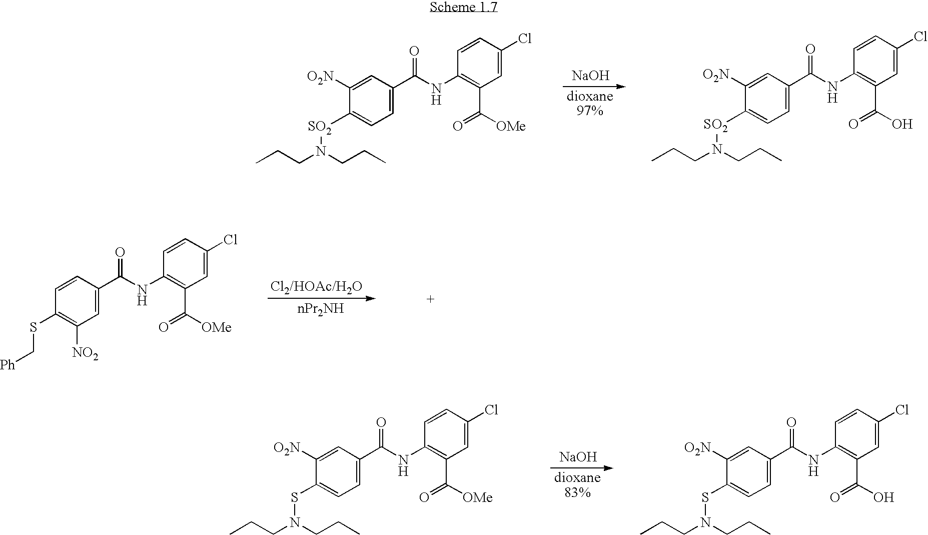

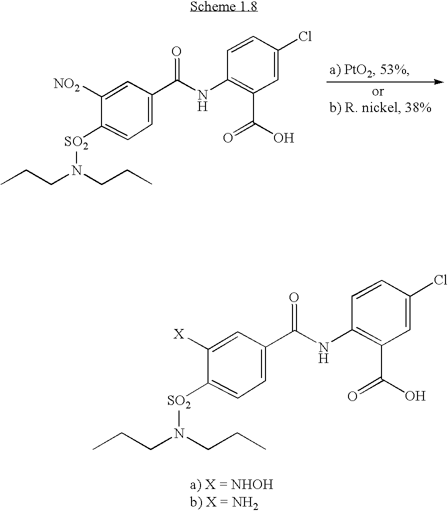

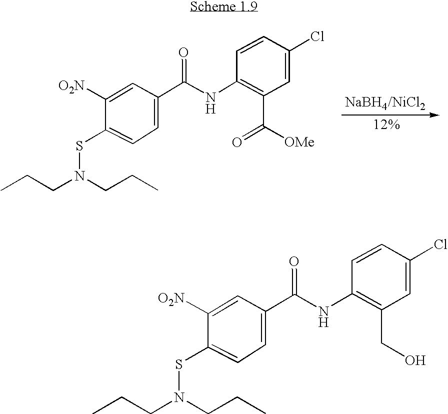

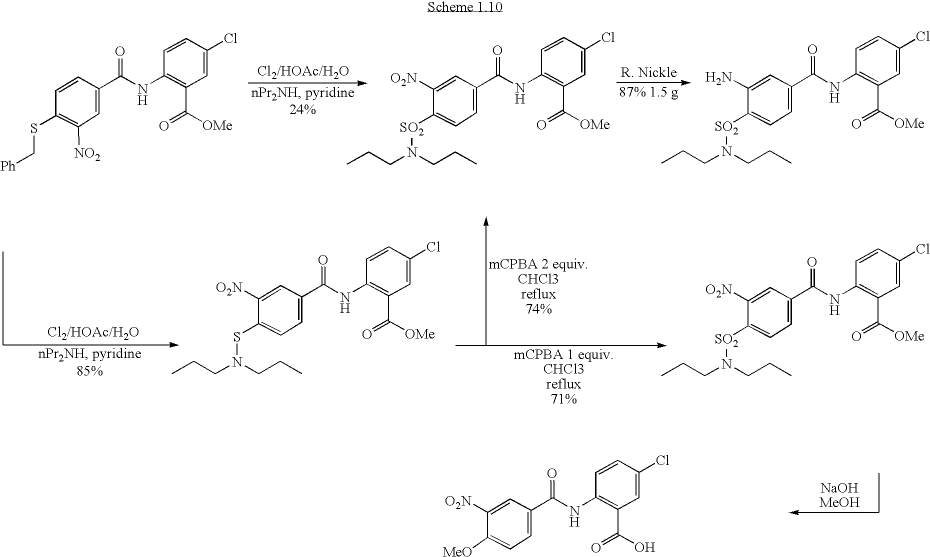

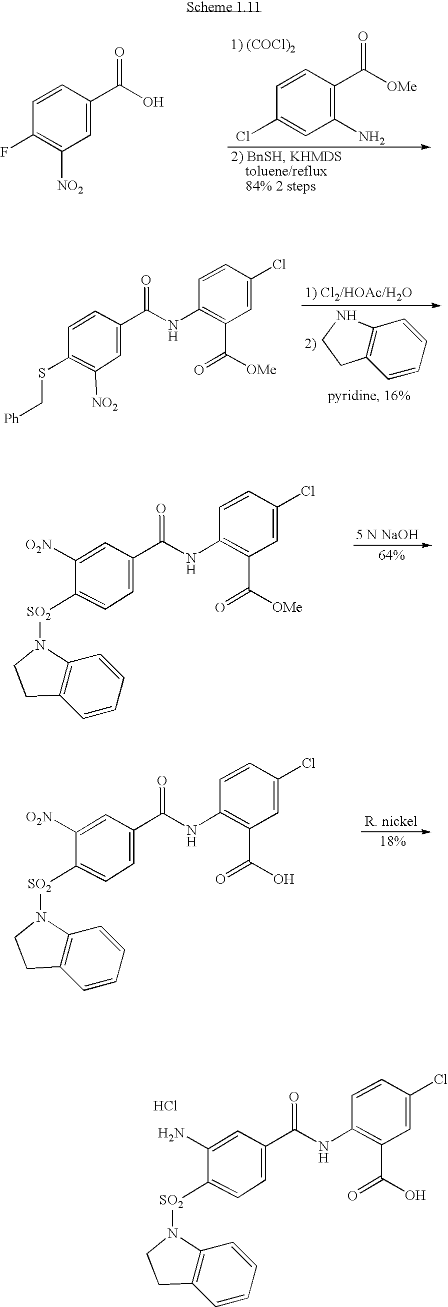

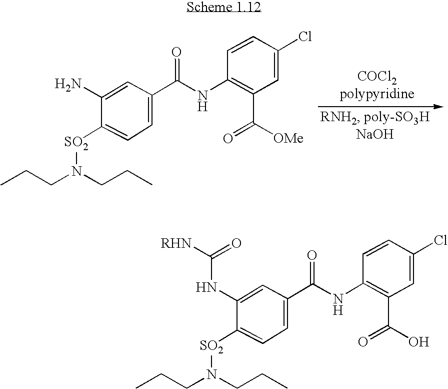

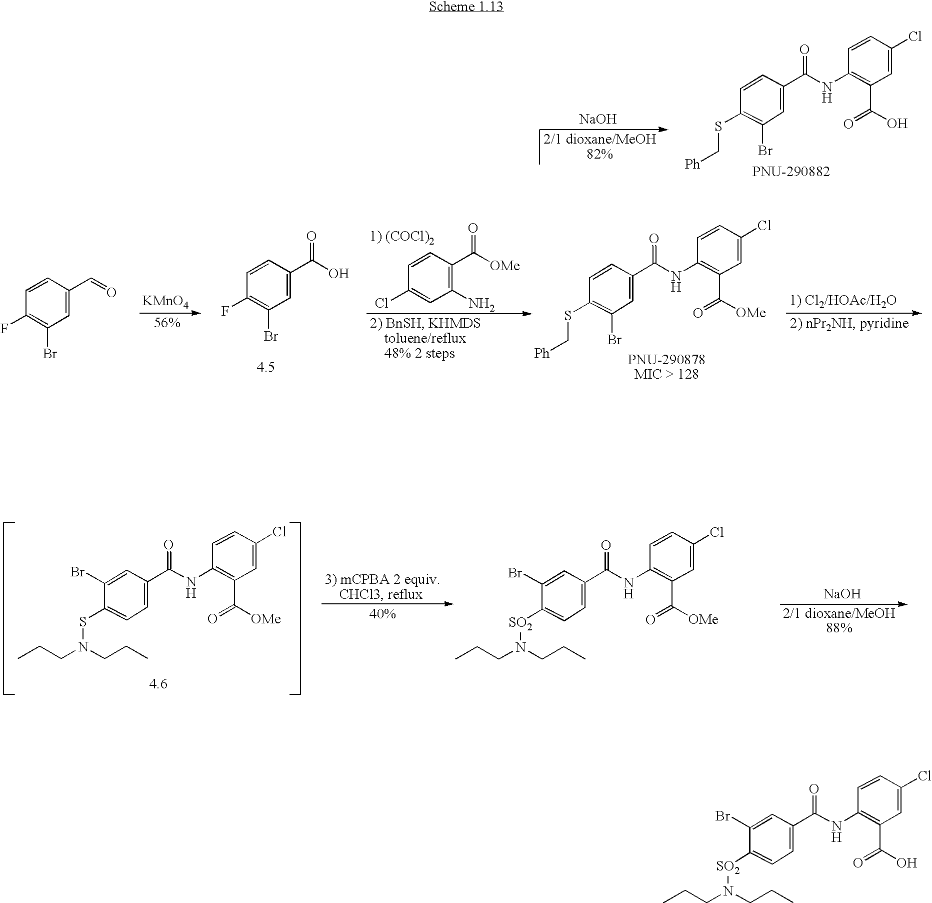

Compounds produced via the above-descirbed synthetic schemes include, but are not limited to, the following:
5-Chloro-2-({4-[(dipropylamino)sulfonyl]benzoyl}amino)benzoic acid
5-Chloro-2-({4-[(dipropylamino)sulfonyl]-3-nitrobenzoyl}amino)benzoic acid
5-Chloro-2-{[4-[(dipropylamino)sulfonyl]-3-(hydroxyamino)benzoyl]amino}benzoic acid hydrochloride
2-({3-Amino-4-[(dipropylamino)sulfonyl]benzoyl}amino)-5-chlorobenzoic acid hydrochloride
2-{[4-(Benzylsulfanyl)-3-nitrobenzoyl]amino}-5-chlorobenzoic acid
5-Chloro-2-({4-[(dipropylamino)sulfanyl]-3-nitrobenzoyl}amino)benzoic acid
Methyl 5-chloro-2-({4-[(dipropylamino)sulfinyl]-3-nitrobenzoyl}amino)benzoate
5-Chloro-2-{[4-(2,3-dihydro-1H-indol-1-ylsulfonyl)-3-nitrobenzoyl]amino}benzoic acid
Cyano 2-{[3-(2,3-dihydro-1H-indol-1-ylsulfonyl)benzoyl]amino}-5-ethynylbenzoic acid
Methyl 2-({3-amino-4-[(dipropylamino)sulfonyl]benzoyl}amino)-5-chlorobenzoate
2-({3-Bromo-4-[(dipropylamino)sulfonyl]benzoyl}amino)-5-chlorobenzoic acid

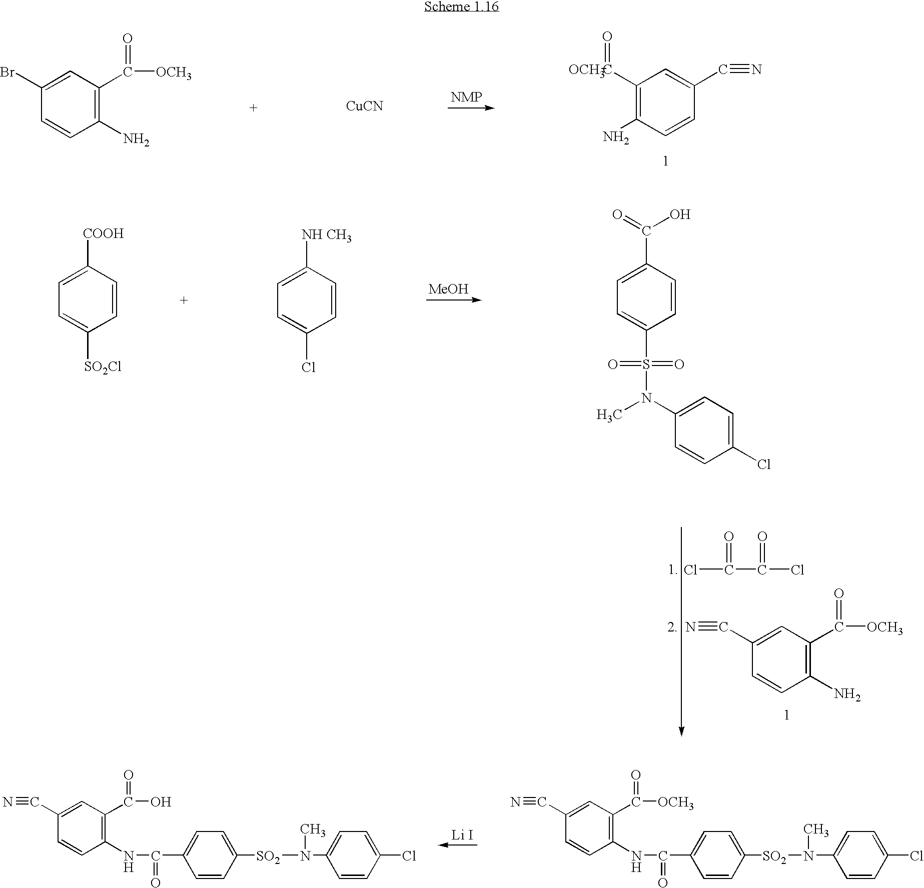

### Preparation of 4-{[4-chloro(methyl)anilino]sulfonyl}benzoic acid

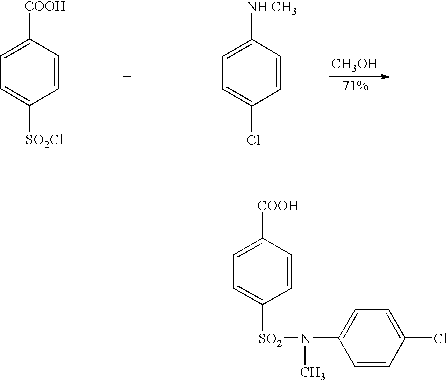

A solution of 4-chloro-N-methylaniline (10.0 g, 0.0706 mol, 1.1 eq) and triethylamine (7.78 g, 0.0770 mol, 1.2 eq) in 140 mL of methanol, cooled in an ice bath at 0-5° C., was treated portionwise over a one minute period with solid 4-chlorosulfonyl benzoic acid (14.2 g, 0.0642 mol, 1.0 eq). After the addition was complete, the cooling bath was removed and the reaction mixture was stirred under a nitrogen atmosphere while warming to room temperature on its own. After 5.5 h, the contents were poured into 270 mL of ice water containing 130 mL of 3 N NaOH, washed the milky solution with methylene chloride (2×100 mL), acidified the aqueous layer with 35 mL of concentrated HCl. After cooling the mixture in an ice bath, the white precipitated product was collected and dried in a vacuum oven at 70° C. overnight to yield 14.92 g (71%) of 2. ¹H NMR (DMSO-d₆) δ 13.53 (brs, 1 H), 8.11 (dd, J=2, 7 Hz, 2 H), 7.63 (dd, J=2, 7 Hz, 2 H), 7.42 (dd, J=2, 7 Hz, 2 H), 7.14 (dd, J=2, 7 Hz, 2 H), 3.15 (s, 3 H) ppm.

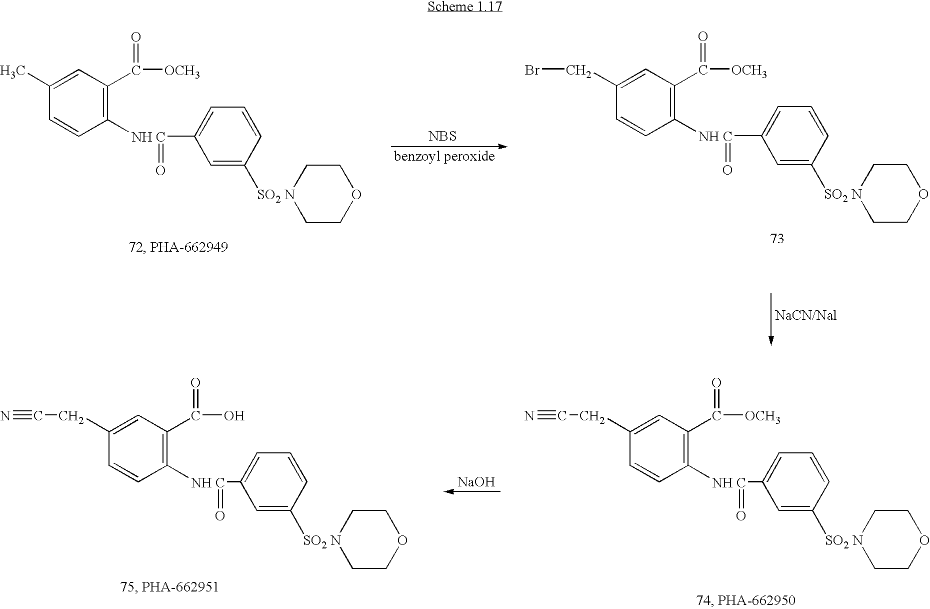

To 21 mL of carbon tetrachloride at room temperature was added benzoyl peroxide (0.095 g, 0.393 mmol, 0.10 eq). The solution was slowly heated to reflux at which time N-bromosuccinimide (0.769 g, 4.32 mmol, 1.1 eq) was added at once followed by a slurry of compound 72 (1.64 g, 3.93 mmol, 1.0 eq) in 9 mL of carbon tetrachloride plus 6 mL of carbon tetrachloride as a rinse. Vigorous refluxing was continued for 2 h, the reaction mixture filtered hot and the solids rinsed with additional hot carbon tetrachloride. The filtrate was concentrated at reduced pressure to give more than theoretical amount of crude bromomethyl compound 73. This was dissolved in 35 mL of acetone, treated with NaCN (0.289 g, 5.90 mmol, 1.5 eq) and NaI (0.029 g, 0.197 mmol, 0.05 eq) and the mixture refluxed for 24 h. An additional 0.50 eq (0.096 g) of NaCN was added and refluxing continued for 3 h longer. The cooled reaction mixture was filtered, the filtrate concentrated at reduced pressure, the residue dissolved in ethyl acetate and washed successively with 10 mL of water and 10 mL of 50% saturated brine. The combined aqueous washings were back extracted once with ethyl acetate, the combined organic extracts dried with anhydrous sodium sulfate and the filtrate concentrated in vacuo. Chromatography with 100 g of silica gel, packed and eluted with acetone-methylene chloride-heptane (1:4:5), afforded cyanomethyl ester 74 in 20% yield (based on 72) as a white solid. Base hydrolysis of 74 (0.297 g, 0.670 mmol) in 4 mL of methylene chloride, 4 mL of methanol and 1 mL of water using 1N NaOH (3.02 mL, 4.5 eq) at room temperature gave a 55% yield of acid 75 as a white solid. 73: TLC (silica gel GF): R_{f}=0.36 acetone-methylene chloride-hexane(1:3:6); ¹H NMR (CDCl₃) δ 8.89 (d, J=7 Hz, 1 H), 8.41 (t, J=1 Hz, 1 H), 8.27 (m, 1 H), 8.14 (d, J=2 Hz, 1 H), 7.97 (m, 1 H), 7.75 (t, J=6 Hz, 1 H), 7.66 (dd, J=2, 6 Hz, 1 H), 4.52 (s, 2 H), 3.98 (s, 3 H), 3.78 (t, J=3 Hz, 4 H), 3.10 (t, J=4 Hz, 4 H) ppm.

Scheme 1.18 outlines the solid phase synthesis of halogenated anthranilic acid substrates 5.

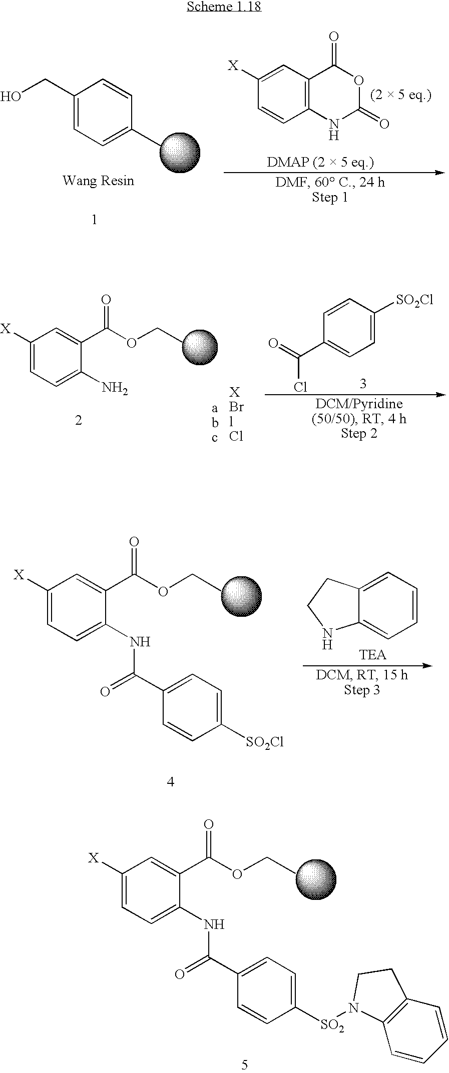

Resin bound iodide 6 was stannylated using the conditions shown in Scheme 10.2. Hunigs base, although not directly involved in the reactions, was used as a proton scavenger. A library based on this template was successfully prepared using Suzuki cross-coupling conditions.

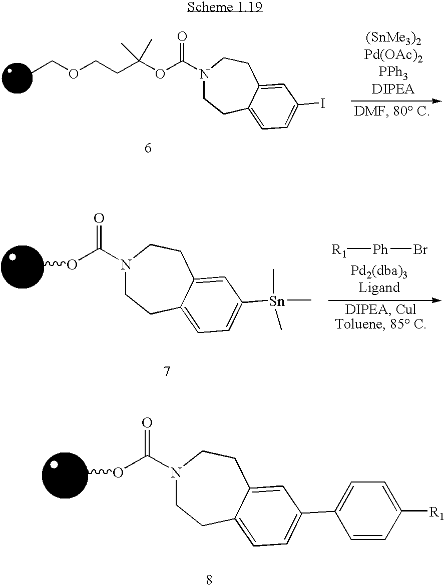

Applying the Stille conditions to the template, stannylated product 9 was prepared from iodide 5b. The reaction was monitored via observance of the protodestannylation product after TFA cleavage from resin. Stannylation of the corresponding solid-phase bromide 5a was less successful.

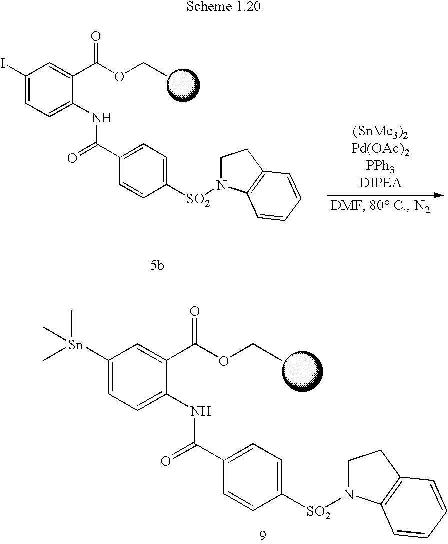

Attempts at coupling aryl bromides and iodides with the stannylated resin gave some product, but not in quantities suitable for library production (Scheme 1.21). Protodestannylation and homocoupling were the major competing reactions, leaving product purities in the 25% range. The reactions were monitored by HPLC (at 210 nm), and product identities were confirmed by LC/MS.

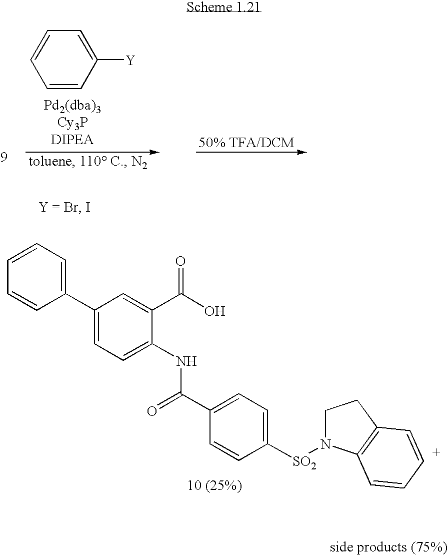

Suzuki coupling chemistry was conducted under the conditions shown in Scheme 1.22.

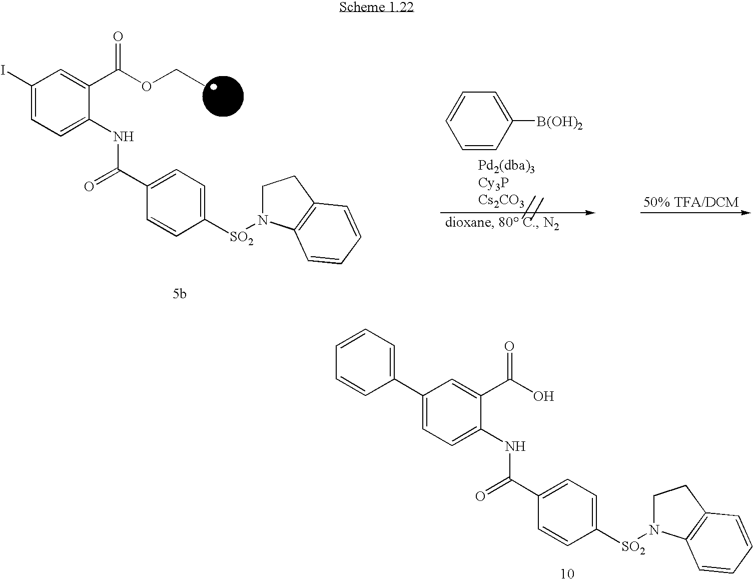

The cross-coupling reaction from the other direction is shown in Scheme 1.23, in which purchased aryl tin compounds were coupled with the resin-bound iodide.

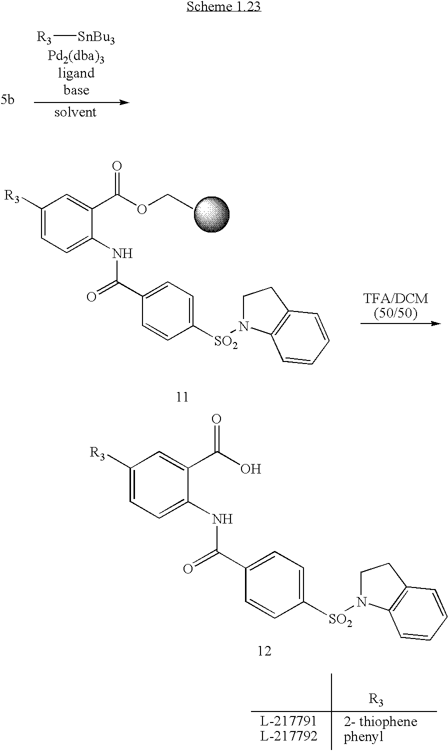

The best results in the case of tributylphenyl tin were obtained in toluene with 1,1′-bis(diphenylphosphino)-ferrocene as ligand and a reaction time of 2.5 hours at 115° C. In the case of 2-(tributylstannyl)thiophene, toluene was the solvent of choice and tricyclohexylphosphine, triphenyl arsine, and 1,1′-bis(diphenylphosphino)-ferrocene worked equally well after 2.5 hours at 115° C.

TABLE 1.1
Commercially Available Aryl Tin Compounds

| | | | |
|---|---|---|---|
| 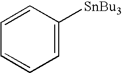 | 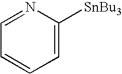 | 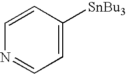 | 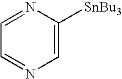 |
| 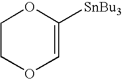 | 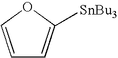 | 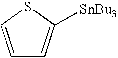 | 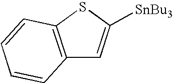 |
| 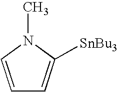 | 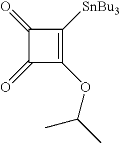 | | |

Installation of Ketones via Palladium-Catalyzed Coupling with Acid Chlorides

Acid chlorides were coupled with 9 (see scheme 1.20) using similar, but milder conditions (Scheme 10.7). The ketone product (13) was produced using triphenylphosphine as ligand and THF as solvent in 75% yield and 70% purity. A carbon monoxide atmosphere was used to eliminate small amounts of the corresponding aryl-aryl product formation (12), while Hunigs base was employed as the proton scavenger to help avoid protodestannylation.

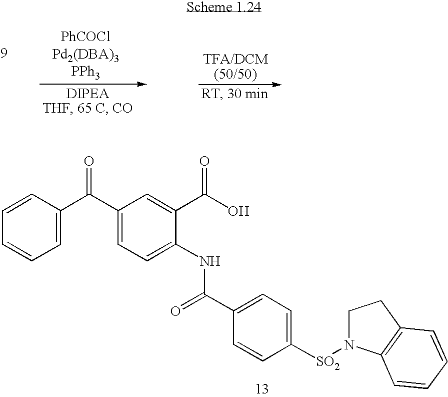

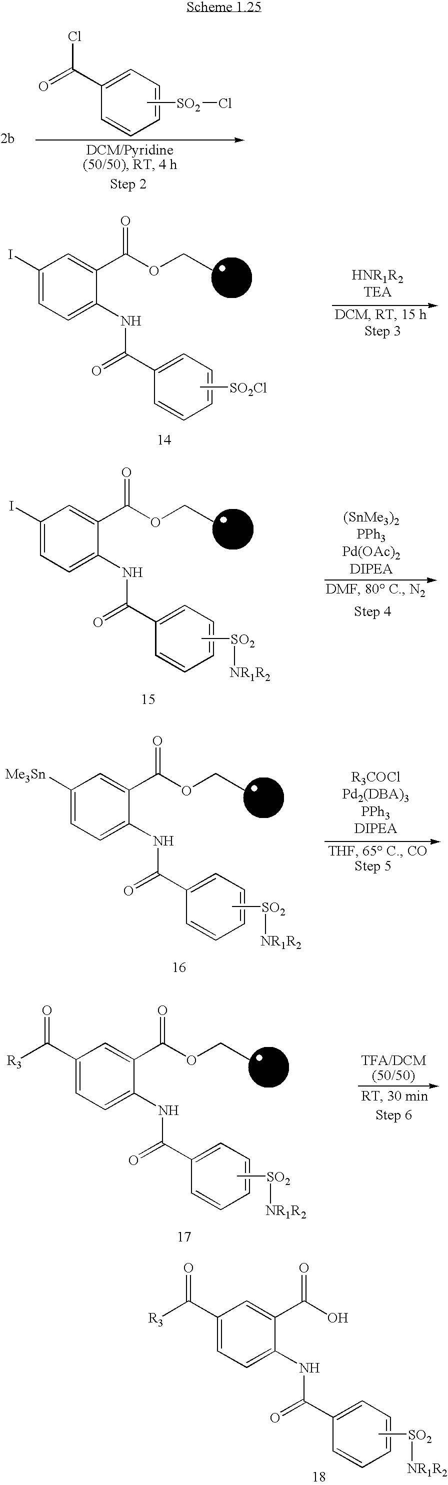

where R3 is a C1-4 alkyl optionally substituted with halo, —OH, CN, and NO₂.

TABLE 1.2
Diversity Elements (no. of ≧ 70% pure products/no. attempted)

| sulfonyl | | | |
|---|---|---|---|
| chlorides | amines | acid chlorides | |
| 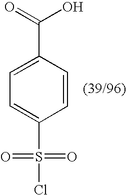 | 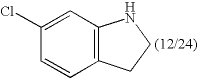 | 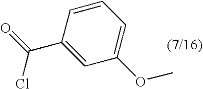 | 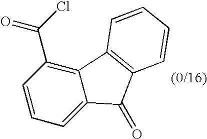 |
| 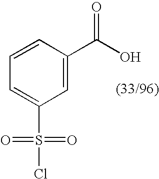 | 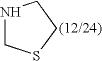 | 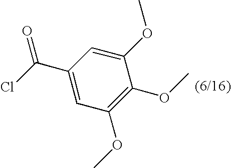 | 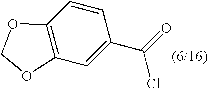 |
| | 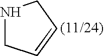 | 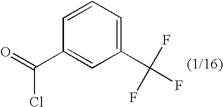 | Iodo (11/16) |
| | 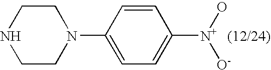 | 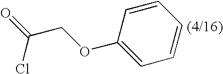 | H (13/16) |
| | 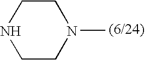 | 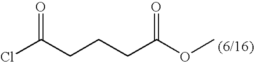 | |
| | 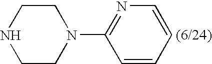 | 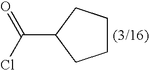 | |
| | 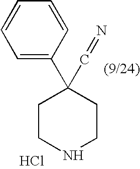 | 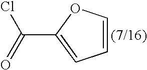 | |
| | 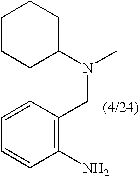 | 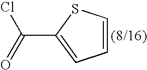 | |

Derivatization of Aryl Ketones: Derivatizing the Ketones as oximes, alkoxyanines, hydrazones, and amines

Oximes and alkoxyamines (20) were prepared in reasonable purities from their corresponding hydroxylamine hydrochlorides and resin 19 in pyridine (Scheme 1.26). Hydrazone, sulfonylhydrazone, and acyl-hydrazone formations (21) using literature conditions, however, were sluggish and could never be pushed to completion.

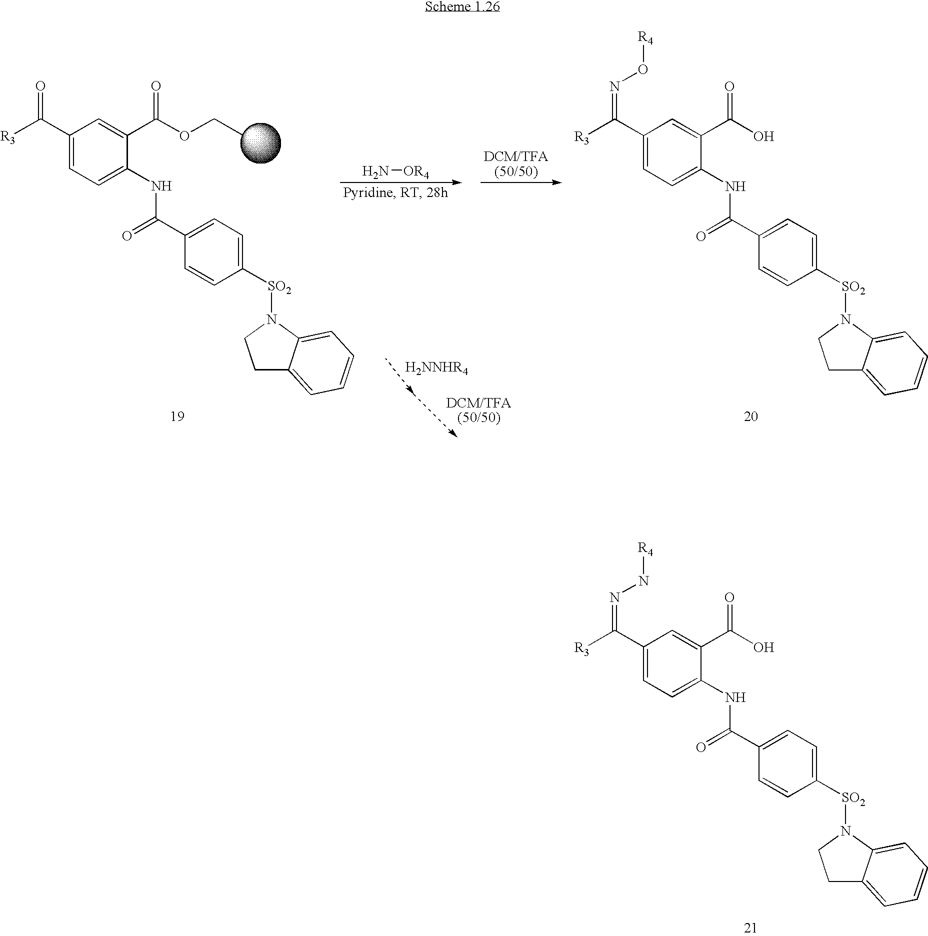

Amines 22 were prepared on solid-phase using reductive amination. Imine formation, mediated by titanium isopropoxide, typically took four to six hours to go to completion. The sodium triacetoxy borohydride reduction was allowed to proceed overnight to give good quality amine products.

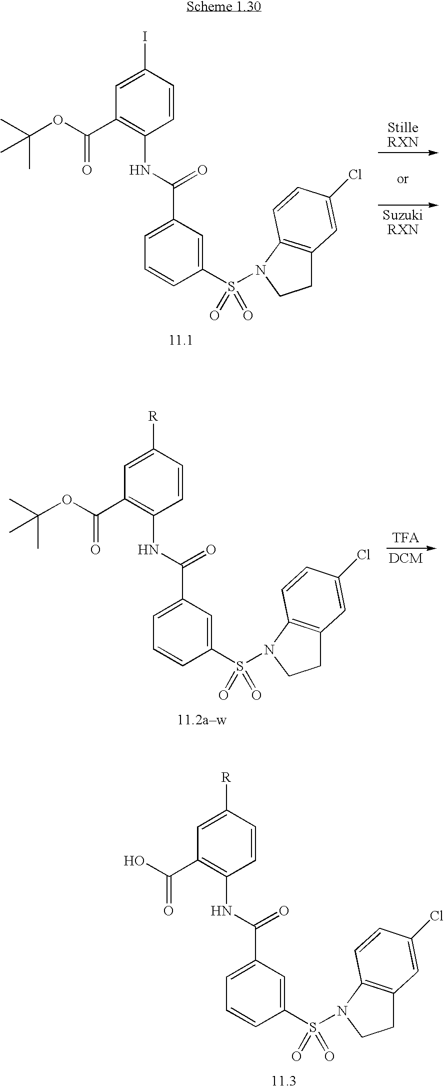

### t-Butyl 2-({3-[(5-chloro-2,3-dihydro-1H-indol-1-yl)sulfonyl]benzoyl}aamino)-5-iodobenzoate, a, Compound 11.1

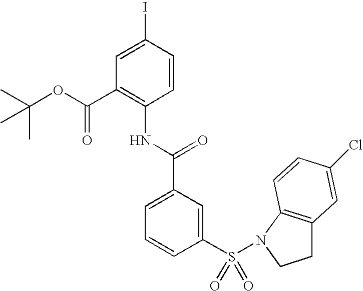

3-[(5-chloro-2,3-dihydro-1H-indol-1-yl)sulfonyl]benzoic acid (2.3 g, 6.9 mmol, 1 equivalent) and oxyl chloride (2.6 g, 20.5 rnmol, 3 equivalent) were dissolved in methylene chloride (30 ml), followed by the addition of DMF (0.4 ml). Gas evolution was observed. The mixture was stirred at room temperature for 2 h later, then heptane (30 ml) was added. The solution was concentrated to dryness, and the residue was re-dissolved in DCM (30 ml), followed by the dropwise addition of PHA-561052 (2.2 g, 6.9 mmol, 1 equivalent) in DCM (20 ml) and pyridine (1.2 ml). The resulting solution was stirred overnight, then diluted with MTBE (200 ml) and washed with 0.1N HCl, 1N NaOH, brine, dried (MgSO₄), filtered, and concentrated in vacuo. The residue was recrystallized from hepane to afford 2.4 g (55%) of 1 as a yellow solid. ¹H NMR (400 MHz, DMSO-d₆) δ 12.34 (s, 1 H), 8.67 (d, J=9.0 Hz, 1 H), 8.54 (s, 1 H), 8.33 (m, 1 H), 8.24 (d, J=8.5 Hz, 1 H), 7.97 (d, J=8.4 Hz, 1 H), 7.88 (d, J=8.5 Hz, 1 H), 7.64 (m, 2 H), 7.17 (d, J=8.5 Hz, 1 H), 7.06 (s, 1 H), 4.08 (t, J=8.5 Hz, 2 H), 2.95 (t, J=8.4 Hz, 2 H), 1.67 (s, 9 H).

### 2-({3-[(5-Chloro-2,3-dihydro-1H-indol-1-yl)sulfonyl]benzoyl}amino)-5-iodobenzoic acid

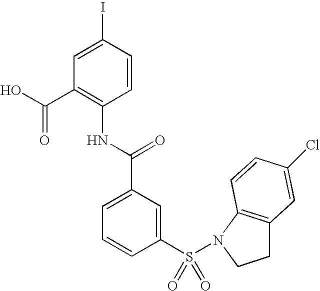 General Method E: (Hydrolysis of the alkyl ester)

Ester 11.1 (150 mg, 0.24 mmol) was dissolved in DCM (6 ml), followed by the addition of TFA (1.2 ml). The solution was shaken overnight, then diluted with DCM (5 ml) and heptane (1 ml). The solution was concentrated in vacuo to dryness, the residue was pumped for about 1 h, then triturated with methanol, filtered to afford 102 mg (75%) of a white solid. ¹H NMR (400 MHz, DMSO-d₆) δ 12.25 (s, 1 H), 8.44 (d, J=9 Hz, 1 H), 8.33 (s, 2 H), 8.31 (m, 1 H), 8.05 (m, 2 H), 7.81 (t, J=8.5 Hz, 1 H), 7.71 (d, J=9 Hz, 1 H), 7.24 (m, 2 H), 4.01 (t, J=8.1 Hz, 2 H), 2.95 (t, 2 H).

### t-Butyl 4-({3-[(5-chloro-2,3-dihydro-1H-indol-1-yl)sulfonyl]benzoyl}amino)[1,1′-biphenyl]-3-carboxylate, 2a

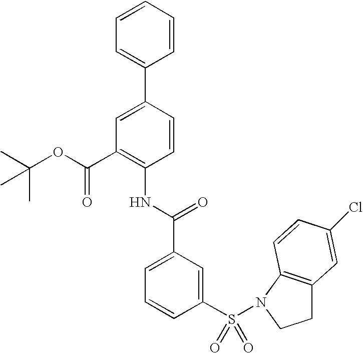 General Method F:

Ester 11.1 (150 mg, 0.235 mmol) and tetrakis(triphenylphosphine) palladium(0) (13.6 mg, 0.01175 mmol) were placed in a 50 ml one-necked round bottom flask. The system was evacuated and filled with argon several times. Then tributylstannylbenzene (91.75 mg, 0.25 mmol) in toluene (10 ml) was added. The resulting solution was heated at 100° C. overnight, cooled to room temperature, then KF (87 mg, ) was added. The mixture was stirred at room temperature for 2 h, filtered through celite. The filtrate was concentrated in vacuo and the residue was purified by silica gel chromatography (EtOAc/heptane 1/25, 1/10) to afford 120 mg (88%) of 11.2a as a yellow solid.

General Method G:

Ester 11.1 (150 mg, 0.235 mmol) and dichlorobis(triphylphosphine) palladium (II) (8.4 mg, 0.012 mmol) were placed in a 50 ml one-necked round bottom flask. The system was evacuated and filled with argon several times. Then tributylstannylbezene (91.7 mg, 0.25 mmol) in THF (10 ml) was added. The resulting solution was heated at 80° C. overnight, cooled to room temperature, KF (87 mg) was added. The mixture was stirred at room temperature for 2 h, filtered through celite. The filtrate was concentrated in vacuo and the residue was purified by silica gel chromatography (EtOAc/heptane 1/25, 1/10) to afford 101 mg (74%) of 11.2a as a yellow solid. ¹H NMR (400 MHz, DMSO-d₆) δ 11.60 (s, 1 H), 8.42 (s, 1 H), 8.35 (D, J=9 Hz, 1 H), 8.27 (d, J=8 Hz, 1 H), 8.15 (d, J=2 Hz, 1 H), 8.06 (d, J=8 Hz, 1 H), 7.98 (d, J=9 Hz, 1 H), 7.84 (t, J=8 Hz, 1 H), 7.70 (d, J=7 Hz, 2 H), 7.50 (m, 3 H), 7.41 (t, J=7 Hz, 1 H), 7.25 (d, J=7 Hz, 2 H), 4.05 (t, J=8 Hz, 2 H), 2.97 (t, J=8 Hz, 2 H), 1.53 (s, 9 H).

### t-Butyl 4′-chloro-4-({3-[(5-chloro-2,3-dihydro-1H-indol-1-yl)sulfonyl]benzoyl}amino)[1,1′-biphenyl]-3-carboxylate, 11.2c

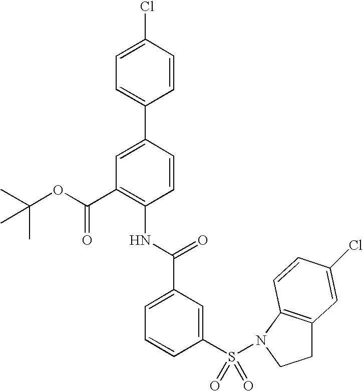 General Method H:

Ester 11.1 (160 mg, 0.25 mmol), tetrakis(triphenylphosphine) palladium(0) (14.5 mg, 0.0125 mmol), sodium carbonate (101 mg, 0.95 mmol) and 4-chlorobenzeneboronic acid (43 mg, 0.275 mmol) were placed in a 100 ml one-necked round bottom flask. The system was evacuated and filled with argon several times. Then THF (50 ml) and distilled water (5 ml) were added. The solution was heated at reflux temperature for 20 h, the solvent was removed in vacuo and residue was purified by silica gel chromatography (EtOAc/hepatane 1/25, 1/10) to get 92 mg (59%) of 11.2c as a yellow solid. ¹H NMR (300 MHz, CDCl₃) δ 12.40 (s, 1 H), 8.93 (d, J=9 Hz, 1 H), 8.59 (s, 1 H), 8.28 (d, J=8 Hz, 1 H), 8.24 (d, J 2.3 Hz, 1 H), 7.95 (d, J=8 Hz, 1 H), 7.80 (dd, J=2.5, 8.2 Hz, 1 H), 7.64 (m, 6 H), 7.20 (d, J=8 Hz, 1 H), 7.08 (s, 1 H), 4.12 (t, J=8 Hz, 2 H), 2.98 (t, J=8 Hz, 2 H), 1.71 (s, 9 H).

### 5-cyano-2-({3-[(1-pyrrolidinylsulfonyl)methyl]benzoyl}amino)benzoic acid PHA-630852

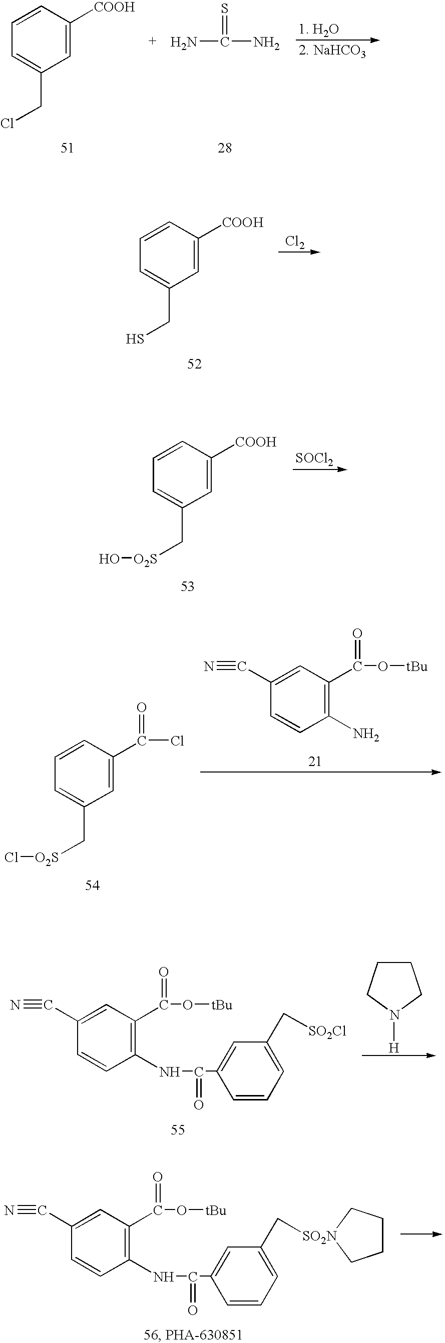

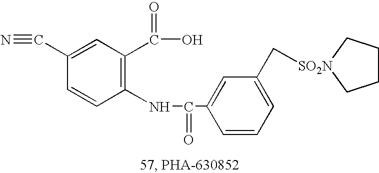

3-chloromethyl)benzoic)acid 51, gave thiomethyl compound 52 in 82% yield⁷. In a manner similar to that described for the preparation of compound 13 above, compound 52 was sequentially treated with gaseous chlorine to obtain the crude sulfonic acid 53 in theoretical yield followed by reaction with thionyl chloride which provided the crude acid chloride 54 as a waxy white solid. This was reacted directly with anthranilate 21 to provide sufficiently pure sulfonyl chloride 55, which was reacted with pyrrolidine to give a 26% yield of ester 56. Subsequent hydrolysis with trifluroacetic acid afforded the acid 57 in 83% yield as a white solid. 57: ¹H NMR (DMSO-d₆) δ 12.48 (s, 1 H), 8.86 (d, J=7 Hz, 1 H), 8.42 (d, J=2 Hz, 1 H), 8.12 (dd, J=2, 7 Hz, 1 H), 8.05 (s, 1 H), 7.95 (d, J=6 Hz, 1 H), 7.72 (d, J=6 Hz, 1 H), 7.64 (t, J=6 Hz, 1 H), 4.58 (s, 2 H), 3.20 (t, J=5 Hz, 4 H), 1.82 (m, 4 H) ppm.

### 2-[(1,3-Benzoxazol-2-ylcarbonyl)amino]-5-cyanobenzoic acid (36310jcr-135a, PHA-734774, SPS #0281864)

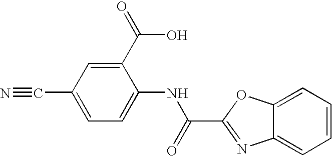

To a solution of benzyl 1,3-benzoxazole-2-carboxylate (233 mg, 0.920 mmol) in 1:1 ethanol/THF (20 mL) was added palladium on carbon (56 mg of 5%, Aldrich) and triethylamine (180 μL, 1.29 mmol, Aldrich). The mixture was stirred under 1 ATM of hydrogen for 2 hours and then filtered through a plug of celite. Removal of the solvent left the triethylamine salt as an orange oil (the protonated form of the acid rapidly decarboxylates and should be avoided). This oil was dissolved in CH₂Cl₂ (20 mL) and treated with DMF (20 μL) followed by oxalyl chloride (220 μL, 2.52 mmol, Aldrich). Solvent and excess oxalyl chloride were removed by rotary evaporation after 76 hours. The residue was dissolved in CH₂Cl₂ (20 mL), and benzyl 2-amino-5-cyanobenzoate (250 mg, 0.991 mmol) in pyridine (8 mL) was added. The mixture was stirred overnight and then added to a separatory funnel with 100 mL of CH₂Cl₂. This solution was washed with 2×100 of 1.0 M HCl and 100 mL of brine. Product was adsorbed onto silica gel and purified on a Biotage Flash 40 M siliga gel cartridge using CH₂Cl₂ as eluent. Product was collected as 218 mg of white solid as the benzyl ester. A mixture of benzyl 2-[(1,3-benzoxazol-2-ylcarbonyl)amino]-5-cyanobenzoate (168 mg, 0.423 mmol) and palladium on carbon (33 mg of 5%, Aldrich) in 2:1 THF/ethanol (30 mL) was stirred under 1 ATM of hydrogen for 25 minutes. The mixture was filtered through a plug of celite and then evaporated. The residue was dried at 100° C. under vacuum yielding 116 mg of white solid. ¹H NMR (400 MHz, DMSO-D6) δ ppm 7.56 (t, J=7.67 Hz, 1 H) 7.63 (t, J=7.88 Hz, 1 H) 7.94 (d, J=8.29 Hz, 1 H) 8.00 (d, J=7.67 Hz, 1 H) 8.16 (dd, J=8.81, 1.97 Hz, 1 H) 8.45 (d, J=2.07 Hz, 1 H) 8.87 (d, J=8.71 Hz, 1 H) 13.16 (s, 1 H).

The following compounds were produced via the methods described above using appropriate starting materials and making non-critical variations.
4-({3-[(5-Chloro-2,3-dihydro-1H-indol-1-yl)sulfonyl]benzoyl}amino)[1,1′-biphenyl]-3-carboxylic acid
2-({3-[(5-Chloro-2,3-dihydro-1H-indol-1-yl)sulfonyl]benzoyl}amino)-5-(2-furyl)benzoic acid
2-({3-[(5-Chloro-2,3-dihydro-1H-indol-1-yl)sulfonyl]benzoyl}amino)-5-(2-thienyl)benzoic acid
2-({3-[(5-Chloro-2,3-dihydro-1H-indol-1-yl)sulfonyl]benzoyl}amino)-5-(2-pyrazinyl)benzoic acid,
2-({3-[(5-Chloro-2,3-dihydro-1H-indol-1-yl)sulfonyl]benzoyl}amino)-5-(1-methyl-1H-pyrrol-2-yl)benzoic acid
4′-Chloro-4-({3-[(5-chloro-2,3-dihydro-1H-indol-1-yl)sulfonyl]benzoyl}amino)[1,1′-biphenyl]-3-carboxylic acid
4-({3-[(5-Chloro-2,3-dihydro-1H-indol-1-yl)sulfonyl]benzoyl}amino)-3′-nitro[1,1′-biphenyl]-3-carboxylic acid
4-({3-[(5-Chloro-2,3-dihydro-1H-indol-1-yl)sulfonyl]benzoyl}amino)-4′-cyano[1,1′-biphenyl]-3-carboxylic acid
2-({3-[(5-Chloro-2,3-dihydro-1H-indol-1-yl)sulfonyl]benzoyl}amino)-5-(5-chloro-2-thienyl)benzoic acid
2-({3-[(5-Chloro-2,3-dihydro-1H-indol-1-yl)sulfonyl]benzoyl}amino)-5-(4-methyl-2-thienyl)benzoic acid
4-({3-[(5-Chloro-2,3-dihydro-1H-indol-1-yl)sulfonyl]benzoyl}amino)-4′-fluoro[1,1′-biphenyl]-3-carboxylic acid
4-({3-[(5-Chloro-2,3-dihydro-1H-indol-1-yl)sulfonyl]benzoyl}amino)-2′-(trifluoromethyl)[1,1′-biphenyl]-3-carboxylic acid
4-({3-[(5-Chloro-2,3-dihydro-1H-indol-1-yl)sulfonyl]benzoyl}amino)-3′,5′-bis(trifluoromethyl)[1,1′-biphenyl]-3-carboxylic acid
2-({3-[(5-Chloro-2,3-dihydro-1H-indol-1-yl)sulfonyl]benzoyl}amino)-5-(5-methyl-2-thienyl)benzoic acid
4-({3-[(5-Chloro-2,3-dihydro-1H-indol-1-yl)sulfonyl]benzoyl}amino)-2′,4′-difluoro[1,1′-biphenyl)-3-carboxylic acid
4′-t-Butyl-4-({3-[(5-chloro-2,3-dihydro-1H-indol-1-yl)sulfonyl]benzoyl}amino)[1,1′-biphenyl]-3-carboxylic acid
4-({3-[(5-Chloro-2,3-dihydro-1H-indol-1-yl)sulfonyl]benzoyl}amino)-3′-trifluoromethyl)[1,1′-biphenyl]-3-carboxylic acid
4-({3-[(5-Chloro-2,3-dihydro-1H-indol-1-yl)sulfonyl]benzoyl}amino)-4′-(trifluoromethyl)[1,1′-biphenyl]-3-carboxylic acid
4-({3-[(5-Chloro-2,3-dihydro-1H-indol-1-yl)sulfonyl]benzoyl}amino)-2′-methyl[1,1′-biphenyl]-3-carboxylic acid
2-({3-[(5-Chloro-2,3-dihydro-1H-indol-1-yl)sulfonyl]benzoyl}amino)-5-(3,5-dimethyl-4-isoxazolyl)benzoic acid
2-({3-[(5-Chloro-2,3-dihydro-1H-indol-1-yl)sulfonyl]benzoyl}amino)-5-(2,4-dimethoxy-5-pyrimidinyl)benzoic acid
2-[(3-{[(4-Chlorophenyl)(methyl)amino]sulfonyl}benzoyl)amino]-5-(trifluoromethyl)benzoic acid,
2-[(3-Bromo-5-{[(4-chlorophenyl)(methyl)amino]sulfonyl}benzoyl)amino]-5-chlorobenzoic acid
5-Bromo-2-[(3-{[(4-chlorophenyl)(methyl)amino]sulfonyl}-5-nitrobenzoyl)amino]benzoic acid
2-({3-[(5-Chloro-2,3-dihydro-1H-indol-1-yl)sulfonyl]benzoyl}amino)-5-cyanobenzoic acid
5-Bromo-2-{[3-cyano-5-(2,3-dihydro-1H-indol-1-ylsulfonyl)benzoyl]amino}benzoic acid
5-Cyano-2-{[3-(2,3-dihydro-1H-indol-1-ylsulfonyl)-5-methylbenzoyl]amino}benzoic acid
Methyl 2-{[3-[2-(acetyloxy)ethyl]-5-(2,3-dihydro-1H-indol-1-ylsulfonyl)benzoyl]amino}-5-cyanobenzoate
5-Cyano-2-{[3-(2,3-dihydro-1H-indol-1-ylsulfonyl)-5-(2-hydroxyethyl)benzoyl]amino}benzoic acid
2-{[3-Bromo-5-(2,3-dihydro-1H-indol-1-ylsulfonyl)benzoyl]amino}-5-chlorobenzoic acid
5-Chloro-2-[(3-{[(4-chlorophenyl)(methyl)amino]sulfonyl}benzoyl)amino]benzoic acid
2-[(3-Bromo-5-{[(4-chlorophenyl)(methyl)amino]sulfonyl}benzoyl)amino]-5-cyanobenzoic acid
5-cyano-2-[(3-{[(2-hydroxyphenyl)(methyl)amino]sulfonyl}benzoyl)amino]benzoic acid
5-Bromo-2-[(5-{[(4-chlorophenyl)(methyl)amino]sulfonyl}-2-methoxybenzoyl)amino]benzoic acid
5-Bromo-2-[(5-{[(4-chlorophenyl)(methyl)amino]sulfonyl}-2-methylbenzoyl)amino]benzoic acid
5-Bromo-2-[(2-bromo-5-{[(4-chlorophenyl)(methyl)amino]sulfonyl}benzoyl)amino]benzoic acid
5-Bromo-2-[(3-{[(4-chlorophenyl)(methyl)amino]sulfonyl}-4-methoxybenzoyl)amino]benzoic acid
5-Bromo-2-[(3-{[(4-chlorophenyl)(methyl)amino]sulfonyl}-4-methybenzoyl)amimo]benzoic acid
5-Bromo-2-[(4-bromo-3-{[(4-chlorophenyl)(methyl)amino]sulfonyl}benzoyl)amino]benzoic acid
2-[(3-{[(4-Chlorophenyl)(methyl)amino]sulfonyl}benzoyl)amino]-5-nitrobenzoic acid 2-[(4-{[(4-Chlorophenyl)(methyl)amino]sulfonyl}benzoyl)amino]-5-nitrobenzoic acid 5-Bromo-2-[(3-{[(4-chlorophenyl)(methyl)amino]sulfonyl}-4-morpholin-4-ylbenzoyl)amino]benzoic acid
5-Bromo-2-[(3-bromo-5-{[(4-chlorophenyl)(methyl)amino]sulfonyl}benzoyl)amino]benzoic acid
2-{[3-Bromo-5-(2,3-dihydro-1H-indol-1-ylsulfonyl)benzoyl]amino}-5-cyanobenzoic acid
2-{[3-Bromo-5-(morpholin-4-ylsulfonyl)benzoyl]amino}-5-chlorobenzoic acid
5-Chloro-2-{[3-(2,3-dihydro-1H-indol-1-ylsulfonyl)-5-methylbenzoyl]amino}benzoic acid
5-Iodo-2-{[3-(morpholin-4-ylsulfonyl)benzoyl]amino}benzoic acid
2-({4-[(5-Chloro-2,3-dihydro-1H-indol-1-yl)sulfonyl]benzoyl}amino)-5-cyanobenzoic acid
2-{[3-(Morpholin-4-ylsulfonyl)benzoyl]amino}-5-thiocyanatobenzoic acid

### Example 2

### Amine, Ether, and Thioether Derivatives

### Preparation of 3-Bromo-4-fluorobenzoic acid

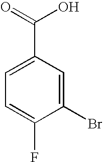

3-Bromo-4-fluoro-benzaldehyde (10.0 g, 49 mmol) in H₂O (150 mL, followed by the addition of KMnO₄ (15.5 g, 98 mmol) heated at reflux (foams extensively) for 1 h, then added additional KMnO₄ (15.5 g, 98 mmol) and continued heating for another 3 h. The reaction was cooled to rt, then filtered through Celite. The solution was acidified with HCl, and the resulting white precipitate was filtered off, to afford 6.1 g (56%) of a white solid.

### Preparation of 3-Anilinobenzoic acid

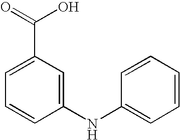

Methyl 3-bromobenzoate (1000 mg, 4.65 mmol), Pd₂(dba)₃ (53 mg, 0.058 mmol), Cs₂CO₃ (2120 mg, 1.4 mmol) and N-[2′-(dicyclohexylphosphino)-1,1′-biphenyl-2-yl]-N,N-dimethylamine (27 mg, 0.07 mmol) were placed in a 100 ml one-necked round bottom flask. The system was evacuated and filled with argon several times. Then aniline (519 mg, 5.58 mmol) was added, followed by the addition of toluene (50 ml). The solution was heated at 100° C. for 20 h, the solvent was removed in vacuo and residue was purified by silica gel chromatography (EtOAc/hepatane 1/3) to get 180 mg (18%) of methyl ester as a yellow solid, which was hydrolyzed by LiOH (50 mg)) in THF (4 ml) and water (1 ml) to afford 140 mg (82%) of 3-Anilinobenzoic acid as a white solid. ¹H NMR (400 MHz, DMSO-d₆) δ 8.02 (s, 1 H), 7.65 (s, 1 H), 7.33 (d, J=7.5 Hz, 1 H), 7.19 (t, J=8.3 Hz, 2 H), 7.10 (d, J=7.7 Hz, 1 H), 7.03 (d, J=7.6 Hz, 2 H), 6.96 (m, 1 H), 6.76 (t, J=7.3 Hz, 1 H);

### 2-[(3-Anilinobenzoyl)amino]-5-cyanobenzoic acid

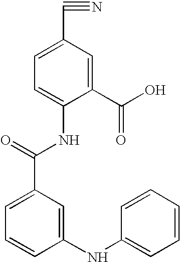

Prepared according to the general methods described above: 3-Anilinobenzoic acid (140 mg, 0.66 mmol) and PHA-561053 (130 mg, 0.59 mmol) afforded 61 mg (25%) of t-butyl ester as a yellow solid, which was hydrolyzed to 48 mg (91 %) of a green solid.

Analytical Data for PHA-610938

¹H NMR (300 MHz, DMSO-d₆) δ 8.81 (d, J=9.0 Hz, 1 H), 8.46 (s, 1 H), 8.35 (d, J=2.2 Hz, 1 H), 7.82 (dd, J=1.9, 8.8 Hz, 1 H), 7.72 (s, 1 H), 7.42 (m, 2 H), 7.27 (m, 3 H), 7.14 (d, J=7.8 Hz, 2 H), 6.88 (t, J=7.3 Hz, 1 H).

### Preparation of 3-[(Pyridin-4-ylmethyl)thio]benzoic acid

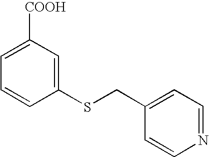

Water (10 mL) was added to a flask containing 3-mercaptobenzoic acid (2.08 g, 13.5 mmol, Aldrich) and sodium hydroxide (1.16 g, 29.0 mmol). To the resulting solution was added 4-picolyl chloride hydrochloride (2.31 g, 14.1 mmol, Aldrich) and ethanol (20 mL). The mixture was heated in a 75° C. oil bath for 1 hour and then added to a separatory funnel with 100 mL of water and 100 mL of CH₂Cl₂. This resulted in a suspension in the aqueous layer. This suspension was washed with an additional 100 mL of CH₂Cl₂ and then filtered. The solid was then dried at 100° C. under vacuum yielding 2.80 g of white solid.

### Preparation of 3-[(Phenylthio)methyl]benzoic acid

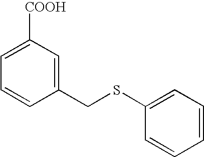

To a solution of the corresponding methyl ester described by Holoboski, M. A.; Koft, E. in J. Org. Chem., 1992, 57, 965-969, (1.23 g, 4.76 mmol) in methanol (15 mL) was added 1.0 M aqueous NaOH (8.0 mL). The resulting mixture was heated in a 50° C. oil bath for 1.5 hours. Most of the methanol was removed by rotary evaporation, and the residue was added to a separatory funnel with 100 mL of 1.0 M aqueous HCl and 100 mL of CH₂Cl₂. The CH₂Cl₂ was washed with another 100 mL of 1.0 M aqueous HCl followed by 100 mL of water and then dried over Na₂SO₄. Solvent was removed, and the residue was dried at 100° C. yielding 1.11 g of white solid.

### 5-Bromo-2-({3-[(phenylthio)methyl]benzoyl}amino)benzoic acid

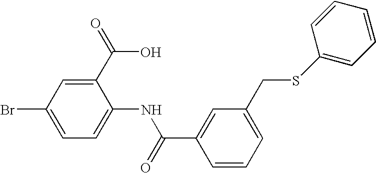

To 3-[(phenylthio)methyl]benzoic acid (400 mg, 1.64 mmol) in CH₂Cl₂ (15 mL) was added DMF (20 μL) and oxalyl chloride (200 μL, 2.29 mmol). The mixture was stirred for 1.5 hours, and the solvent and excess oxalyl chloride were removed by rotary evaporation. The residue was dissolved in CH₂Cl₂ (15 mL), and methyl 2-amino-5-bromobenzoate (330 mg, 1.43 mmol, Avocado) in pyridine (8 mL) was added. The mixture was stirred overnight and then added to a separatory funnel with 100 mL of CH₂Cl₂. This solution was washed with 2×100 mL of 1 M aqueous HCl and 100 mL of brine. The CH₂Cl₂ was evaporated in the presence of silica gel, and the product was purified by chromatography using a Biotage Flash 40 M silica cartridge with a gradient from 50% CH₂Cl₂/heptane to 75% CH₂Cl₂/heptane as eluent. Yield was 544 mg of white solid as the methyl ester.

To a mixture of the corresponding methyl ester (386 mg, 0.845 mmol) in dioxane (20 mL) was added 1 M aqueous sodium hydroxide (2.0 mL). The mixture was stirred for at room temperature for 1.25 hours and then at 50° C. for 1.5 hours. The reaction mixture was added to a separatory funnel with 100 mL of 1 M aqueous HCl, and the product was extracted into 100 mL of CH₂Cl₂. The CH₂Cl₂ was washed with an additional 100 mL of 1 M aqueous HCl followed by 100 mL of brine. It was then dried over Na₂SO₄ and evaporated. The residue was recrystallized from hot ethanol (8 mL). The solids were washed with ethanol followed by heptane and then dried at 100° C. under vacuum yielding 279 mg of white solid. ¹H NMR (400 MHz, DMSO-d₆) δ 12.08 (s, 1 H), 8.64 (d, J=9.2 Hz, 1 H), 8.12 (d, J=2.5 Hz, 1 H), 7.97 (s, 1 H), 7.86 (dd, J=9.2, 2.5 Hz, 1 H), 7.80 (d, J=7.6 Hz, 1 H), 7.61 (d, J=7.6 Hz, 1 H), 7.51 (t, J=7.6 Hz, 1 H), 7.35 (d, J=7.1 Hz, 2 H), 7.29 (t, J=7.9 Hz, 2 H), 7.18 (t, J=7.1 Hz, 1 H), 7.35 (s, 2 H).

Other compounds produced via the above-described methodology using appropriate starting materials and maiking non-critical variations include:
2-{[3-(benzylthio)benzoyl]amino}-5-bromobenzoate
2-{[3-(Benzyloxy)benzoyl]amino}-5-bromobenzoic acid
5-Bromo-2-{[3-(ethylthio)benzoyl]amino}benzoic acid
Methyl-5-Bromo-2-({3-[(pyridin-4-ylmethyl)thio]benzoyl}amino)benzoate
5-Bromo-2-({3-[(pyridin-4-ylmethyl)thio]benzoyl}amino)benzoic acid
5-bromo-2-({3-[(pyridin-4-ylmethyl)thio]benzoyl}amino)benzoic acid hydrochloride
5-Bromo-2-[(3-phenoxybenzoyl)amino]benzoic acid
5-Bromo-2-{[3-(phenylthio)benzoyl]amino}benzoic acid
5-Cyano-2-[(3-phenoxybenzoyl)amino]benzoic acid
5-Cyano-2-({3-[(pyridin-4-ylmethyl)thio]benzoyl}amino)benzoic acid
5-Cyano-2-({3-[(pyridin-4-ylmethyl)thio]benzoyl}amino)benzoic acid hydrochloride
2-{[3-(Benzyloxy)benzoyl]amino}-5-cyanobenzoic acid
2-{[3-(Benzylthio)benzoyl]amino}-5-cyanobenzoic acid
5-cyano-2-({3-[(1-phenylethyl)thio]benzoyl}amino)benzoic acid
5-cyano-2-{[3-(cyclopentylthio)benzoyl]amino}benzoic acid
5-cyano-2-{[3-(cyclopentylsulfinyl)benzoyl]amino}benzoic acid
5-Chloro-2-[(4-methoxy-3-nitrobenzoyl)amino]benzoic acid
2-{[4-(Benzylsulfanyl)-3-bromobenzoyl]amino}-5-chlorobenzoic acid
5-Cyano-2-{[3-(3-fluorophenoxy)benzoyl]amino}benzoic acid
5-Cyano-2-{[3-(2-methylphenoxy)benzoyl]amino}benzoic acid
5-Cyano-2-{[3-(4-methoxyphenoxy)benzoyl]amino}benzoic acid
5-Cyano-2-{[3-(3-nitrophenoxy)benzoyl]amino}benzoic acid

### Example 3

### Ketone Derivatives

### 2-[(3-Benzoylbenzoyl)amino]-5-bromobenzoic acid

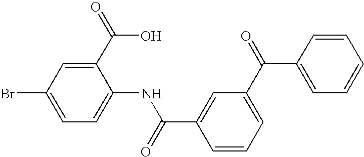

To 3-benzoylbenzoic acid (633 mg, 2.80 mmol, Aldrich) in CH₂Cl₂ (20 mL) was added DMF (20 μL) and oxalyl chloride (450 μL, 5.16 mmol). The mixture was stirred for 1.7 hours, and the solvent and excess oxalyl chloride were removed by rotary evaporation. The residue was dissolved in CH₂Cl₂ (20 mL), and methyl 2-amino-5-bromobenzoate (565 mg, 2.46 mmol, Avocado) in pyridine (6 mL) was added. The mixture was stirred overnight and then added to a separatory funnel with 100 mL of CH₂Cl₂. This solution was washed with 2×100 mL of 1 M aqueous HCl and 100 mL of brine. The CH₂Cl₂ was evaporated in the presence of silica gel, and the product was purified by chromatography using a Biotage Flash 40 M silica cartridge with a gradient from 75% CH₂Cl₂/heptane to 100% CH₂Cl₂ as eluent. Yield was 825 mg of white solid as the methyl ester. To a mixture of the corresponding methyl ester (645 mg, 1.47 mmol) in dioxane (20 mL) was added 1 M aqueous sodium hydroxide (3.0 mL). The mixture was stirred in a 50° C. oil bath for 2 hours. The reaction mixture was added to a separatory funnel with 100 mL of 1 M aqueous HCl, and the product was extracted into 100 mL of CH₂Cl₂. The organics were washed with an additional 100 mL of 1 M aqueous HCl followed by 100 mL of water. They were then dried over MgSO₄ and evaporated. The residue was recrystallized from hot ethanol/THF. The solids were washed with ethanol followed by pentane and then dried at 100° C. under vacuum yielding 329 mg of white solid. ¹H NMR (400 MHz, DMSO-d₆) δ 12.17 (s, 1 H), 8.61 (d, J=9.2 Hz, 1 H), 8.31 (s, 1 H), 8.23 (d, J=7.6 Hz, 1 H), 8.12 (d, J=2.0 Hz, 1 H), 7.99 (d, J=7.6 Hz, 1 H), 7.87 (dd, J=9.2, 2.5 Hz, 1 H), 7.77-7.82 (m, 3 H), 7.73 (t, J=7.4 Hz, 1 H), 7.61 (t, J=7.6 Hz, 2 H).

### 5-Bromo-2-({3-[hydroxy(phenyl)methyl]benzoyl}amino)benzoic acid

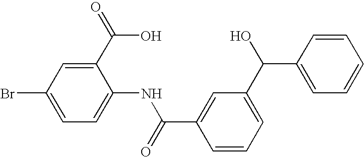

Solid sodium borohydride (82 mg, 2.2 mmol) was added in one portion to a slurry of methyl 2-[(3-benzoylbenzoyl)amino]-5-bromobenzoate (826 mg, 1.88 mmol) in 40 mL of 1:1 methanol/THF. The mixture was stirred for 75 minutes before being quenched by the addition of 1 M aqueous HCl (50 mL). The organics were removed by rotary evaporation, and the product was extracted into 100 mL+50 mL of CH₂Cl₂. The CH₂Cl₂ was evaporated in the presence of silica gel, and the product was purified by chromatography using a Biotage Flash 40 M silica cartridge with a gradient from CH₂Cl₂ to 5% EtOAc/CH₂Cl₂ as eluent. Yield was 433 mg of white solid as the methyl ester. To a mixture of the corresponding methyl ester (348 mg, 0.788 mmol) in dioxane (20 mL) was added 1 M aqueous sodium hydroxide (1.5 mL). The mixture was stirred at room temperature overnight and then heated in a 50° C. oil bath for 30 minutes. The reaction mixture was added to a separatory funnel with 100 mL of 1 M aqueous HCl, and the product was extracted into 100 mL of CH₂Cl₂. The organics were washed with an additional 100 mL of 1 M aqueous HCl followed by 100 mL of water. They were then dried over MgSO₄ and evaporated. The residue was recrystallized from hot ethanol (10 mL). The solids were washed with ethanol followed by pentane and then dried at 100° C. under vacuum yielding 130 mg of white solid. ¹H NMR (400 MHz, DMSO-d₆) δ 12.12 (s, 1 H), 8.66 (d, J=8.7 Hz, 1 H), 8.13 (d, J=2.5 Hz, 1 H), 8.05 (s, 1 H), 7.85 (dd, J=9.2, 2.5 Hz, 1 H), 7.79 (d, J=7.6 Hz, 1 H), 7.62 (d, J=8.1 Hz, 1 H), 7.51 (t, J=7.6 Hz, 1 H), 7.42 (d, J=7.1 Hz, 2 H), 7.32 (t, J=7.6 Hz, 2 H), 7.22 (t, J=7.1 Hz, 1 H), 6.07 (br s, 1 H), 5.81 (s, 1 H).

### 5-Bromo-2-({3-[(methoxyimino)(phenyl)methyl]benzoyl}amino)benzoic acid (PHA-522146)

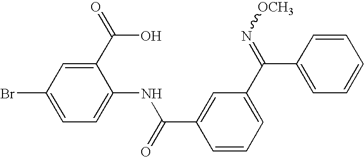

Methyl 2-[(3-benzoylbenzoyl)amino]-5-bromobenzoate (763 mg, 1.74 mmol) was dissolved in 60 mL of 1:1 EtOH/pyridine with warming. After this solution was allowed to cool, solid O-methylhydroxylamine hydrochloride (350 mg, 4.19 mmol, Aldrich) was added in one portion. The resulting slurry was stirred at room temperature for 6 days, after which it was a solution. The solvents were removed by rotary evaporation, and the residue was dissolved in 100 mL of CH₂Cl₂. This solution was washed with 2×100 mL of 1 M aqueous HCl and 100 mL of brine. The CH₂Cl₂ was dried over MgSO₄ and evaporated leaving 785 mg of white solid that was approximately a 1:1 mixture of oxime isomers by 1 H NMR. To a mixture of the corresponding methyl ester (470 mg, 1.01 mmol) in dioxane (15 mL) was added 1 M aqueous sodium hydroxide (2.0 mL). The mixture was stirred at room temperature overnight. The reaction mixture was added to a separatory funnel with 100 mL of 1 M aqueous HCl, and the product was extracted into 100 mL of CH₂Cl₂. The organics were washed with an additional 100 mL of 1 M aqueous HCl followed by 100 mL of water. They were then dried over MgSO₄ and evaporated. The orange residue was recrystallized from hot ethanol (10 mL). The solids were washed with ethanol followed by heptane and then dried at 100° C. under vacuum yielding 255 mg of white solid that was approximately a 1:1 mixture of oxime isomers by 1H NMR. Due to the presence of 2 isomers, the NMR is difficult to assign. At 400 MHz in DMSO-d₆, the amide protons appear as singlets at 12.10 and 12.07 ppm. The aromatic protons appear between 7.32 and 8.63 ppm. The methyl peaks appear as singlets at 3.93 and 3.92 ppm.

### 5-cyano-2-{[3-(cyclopentylcarbonyl)benzoyl]amino}benzoic acid

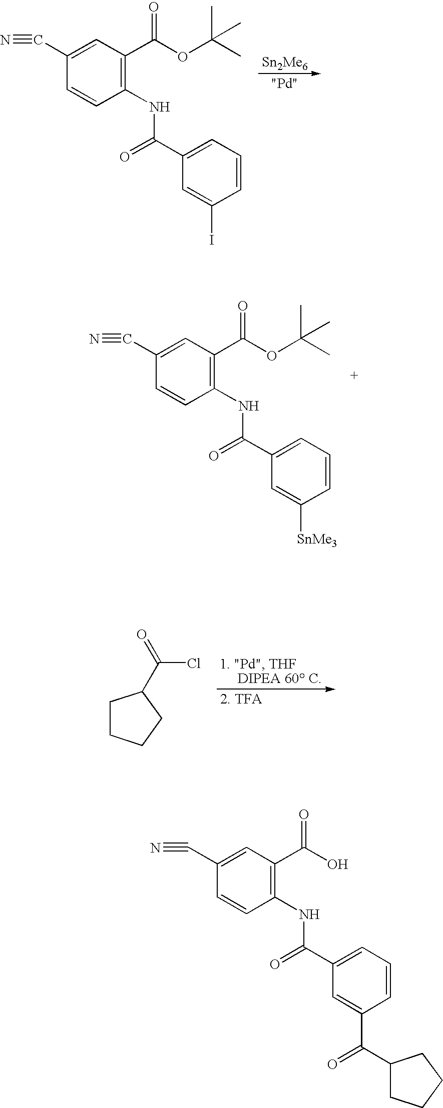

tert-Butyl 5-cyano-2-[(3-iodoberizoyl)amino]benzoate (1.0 g, 2.23 mmol) was dissolved in 20 ml of CH₂Cl₂. Hexamethylditin (1.1 g, 3.35 mmol) and allylpalladium chloride dimer (73 mg, 0.2 mmol) were then added and the mixture stirred at room temperature for 5 hr. The reaction was diluted with CH₂Cl₂ then washed with water. The organic solution was dried over Na₂SO₄ and concentrated in vacuo. The remaining oil was purified via silica gel chromatography to give 670 mg (62%) of the desired tin compound. This product was subsequently dissolved in 15 mL of THF. To this was added DIPEA (1 mL), Pd₂dba₃ (115 mg, 0.125 mmol) and cyclopentanecarbonyl chloride (230 mg 1.73 mmol). The reaction was then warmed to 60° C. and stirred for 10 additional hr. After cooling to room temperature the reaction was poured into 1 M HCl (20 mL) and extracted with EtOAc (3×20 mL). The organic solution was dried over Na₂SO₄ and concentrated in vacuo. The remaining residue was purified via silica gel chromatography, giving 415 mg (72%) of the desired ketone. The ketone was treated with CH₂Cl₂/TFA and stirred for 10 additional hours. The solvent was removed in vacuo and the remaining solid was recrystalized from MeOH to give the title compound (329 mg, 91%) as a white solid. 1H NMR (400 MHz, DMSO) 1.62-1.67 (m, 4H), 1.73-1.80 (m, 2H), 1.92-1.98 (m, 2H), 3.90 (quint, 1H), 7.77 (t, 1H), 8.11 (dd, 1H), 8.19 (d, 1H), 8.27 (d, 1H), 8.41 (d, 1H), 8.53 (s, 1H), 8.84 (d, 1H), 12.55 (s, 1H)

Other compounds produced via the above-described methodology using appropriate starting materials and maiking non-critical variations include:
2-[(3-Benzoylbenzoyl)amino]-5-chlorobenzoic acid
2-[(4-Acetylbenzoyl)amino]-5-bromobenzoic acid
2-[(4-Benzoylbenzoyl)amino]-5-bromobenzoic acid
2-[(3-Acetylbenzoyl)amino]-5-bromobenzoic acid
5-Bromo-2-({3-[(hydroxyimino)phenyl)methyl]benzoyl}amino)benzoic acid
(+)-5-Bromo-2-({3-[hydroxy(phenyl)methyl]benzoyl}amino)benzoic acid
(−)-5-bromo-2-({3-[hydroxy(phenyl)methyl]benzoyl}amino)benzoic acid
2-[(3-Benzoylbenzoyl)amino]-5-nitrobenzoic acid
2-[(3-Benzoylbenzoyl)amino]-5-cyanobenzoic acid
5-Cyano-2-({3-[(hydroxyimino)(phenyl)methyl]benzoyl}amino)benzoic acid
5-Cyano-2-({3-[(methoxyimino)(phenyl)methyl]benzoyl}amino)benzoic acid
   Solid Phase Synthesis

Additional methodologies for producing compounds of this invention are shown below. 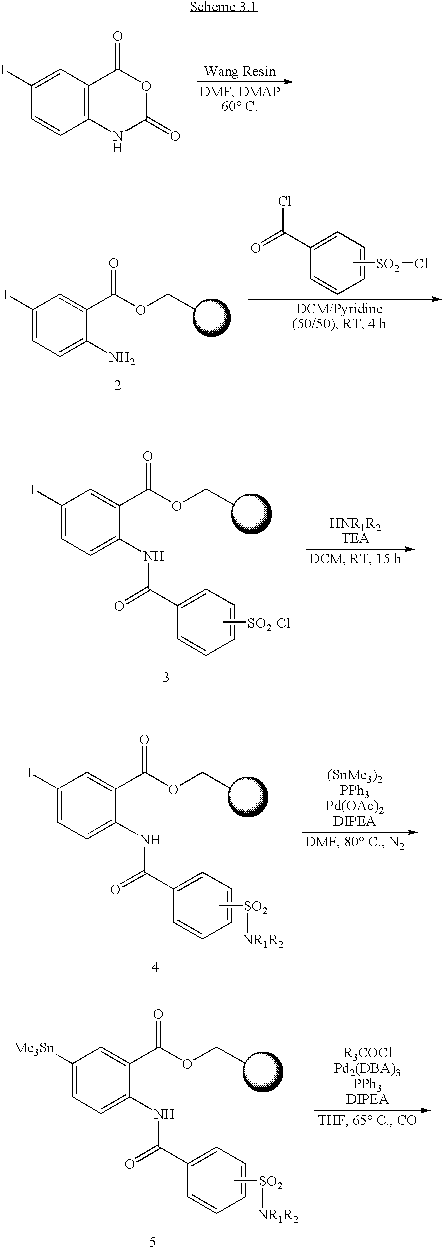 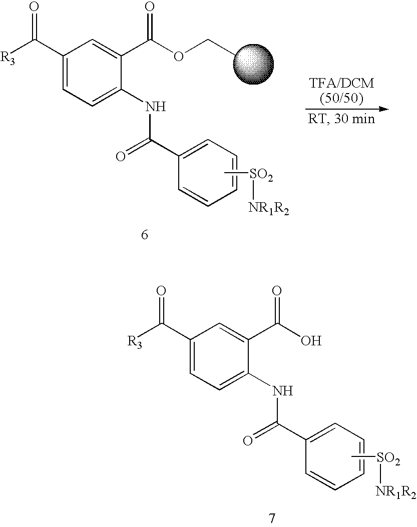
R₃ is a C₁₋₄alkyl optionally substituted with 1-3 halo, —OH, NO₂, or —CN.

Development of a solid phase route to ketones 1 was effected by a similar route and is summarized in Scheme 3.2. Chlorine was selected as the anthranilic acid 5-substituent instead of the 5-bromine of the ketone leads in order to avoid the potential for competing reactions in the ensuing palladium-catalyzed stannylation. Solid-supported aryl halide 8 was prepared by reaction of chloroisatoic anhydride with Wang resin. Coupling with halo (X═Br or I) aroyl chlorides then afforded benzamides 9, which were stannylated with hexamethyl distannane under the influence of palladium catalyst using the same conditions that were applied in Scheme 3.1. The subsequent carbonylation reactions were found to be optimal using the slightly modified conditions of Ellman.⁸ Eliminating the ligand altogether and adding potassium carbonate as another proton scavenger slightly enhanced the rate of the reactions and the product purities in the end. Carbon monoxide was not necessary to eliminate aryl-aryl coupling by-products. One other modification in the synthetic conditions was to decrease the amount of TFA used in the cleavage cocktail in order to avoid trace amounts of a cleavage impurity. 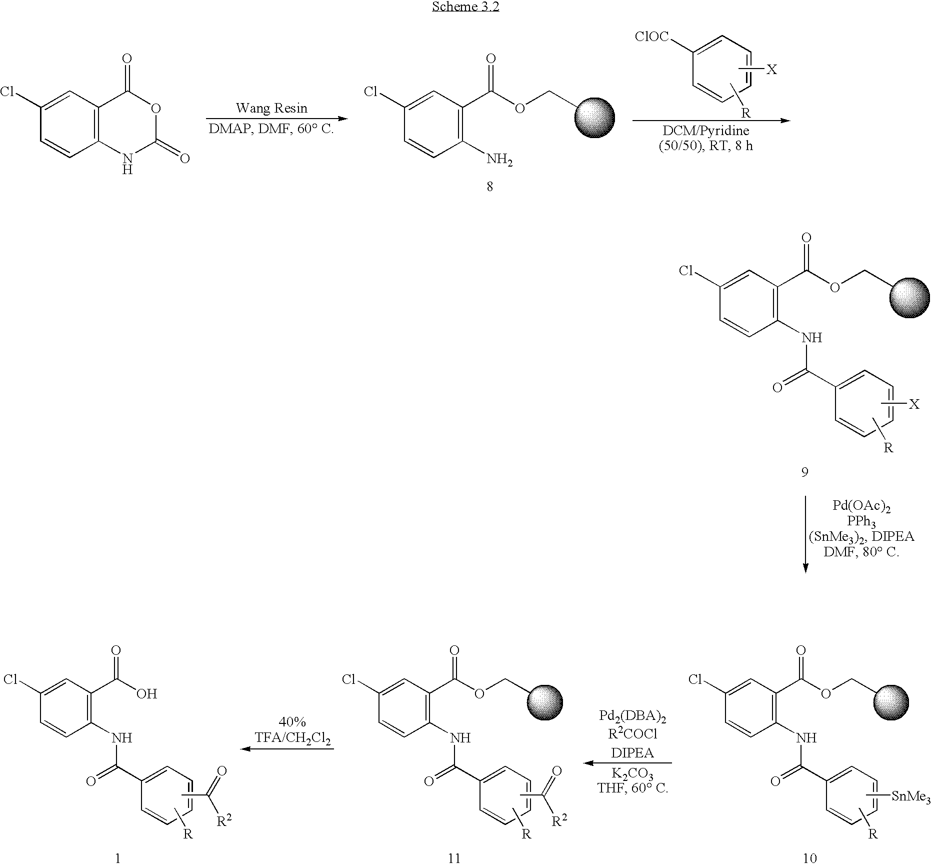
Generation of Oximes and Amines from Solid-Supported Ketones

Chemistry was developed for amine (12) and oxime (13) derivatization of the ketones on solid-phase (Scheme 3.3). Following TFA cleavage (Scheme 3.4), the amines could be successfully purified by trapping the products on sulfonic acid resin and then washing off with 2 N NH₄OH/methanol. The remaining compounds were subjected to preparative HPLC purification. 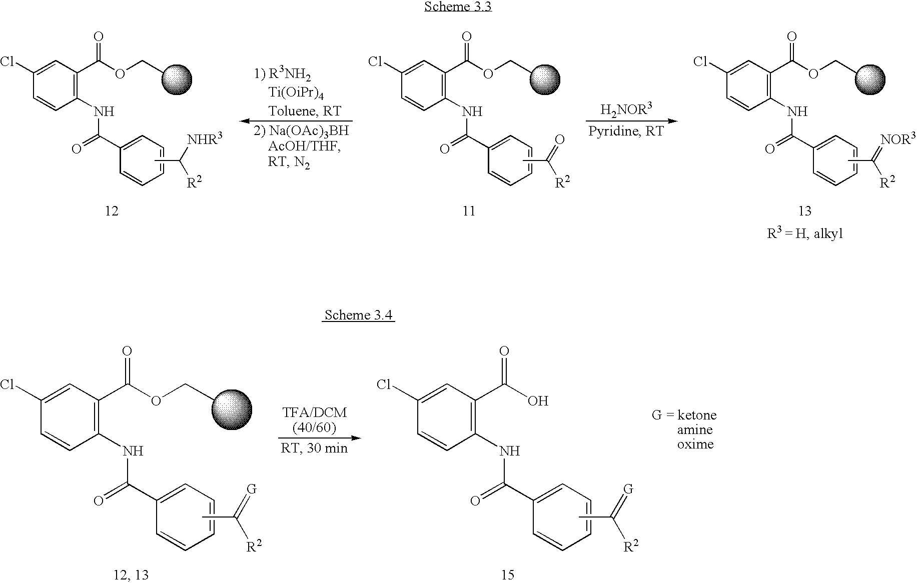
Ketones
Step 1: Preparation of 8

To 4.5 grams of Wang resin (Irori Unisphere, 1.36 mmol/g loading, 6.12 mmol) in a 125 mL serum bottle, 60 mL of DMF were added followed by 6.1 grams (5 eq., 30.6 mmol) of 5-chloroisotoic anhydride and 3.74 grams (5 eq., 30.6 mmol) of 4-dimethylaminopyridine. The serum bottle was purged with nitrogen, capped, and shaken on an orbital mixer at 60° C. Initially, the reagent cocktail was not homogeneous, but after several hours, a concentrated solution had formed around the swelled resin. After 18 hours, the reaction slurry was cooled and transferred to a 60 mL syringe-barrel reaction vessel. The reagent cocktail was then drained and the resin washed as follows: 3× (acetonitrile, DMF), then 3× (acetonitrile, methylene chloride). The resin was treated a second time with 60 mL of DMF, 6.1 grams (5 eq., 30.6 mmol) of 5-chloroisotoic anhydride, and 3.74 grams (5 eq., 30.6 mmol) of 4-dimethylaminopyridine. Following mixing at 60° C. for 6 hours, the reagent cocktail was again drained and the resin washed as above. In a vacuum oven at 25° C., the resin was dried for 72 hours to give a final weight of 5.36 grams (1.14 mmol/g loading).

Step 2: Preparation of 9

To 6.7 mmol of the halo benzoic acids suspended in 20 mL of methylene chloride, 20 μL of DMF and 1.17 mL (1.7 grams, 13.4 mmol, 2 eq.) of oxalyl chloride were added. The flasks were sealed and stirred with occasional release of gas build-up. After stirring overnight, the reaction mixtures had become almost completely homogeneous with no more gas build-up. Solvent and excess oxalyl chloride were then evaporated in vacuo to dryness. The acid chlorides were re-dissolved in IO mL of methylene chloride and added to 1 gram of resin 8 (1.14 mmol/gram loading, 1.14 mmol) swollen with 10 mL of pyridine in 25 mL vials. Some fuming was observed initially. The mixtures were purged with nitrogen for 10 seconds then the vials capped, and the mixtures shaken at room temperature for 4 hours. By that time, the resins had taken on a light orange color and a tan precipitate had formed in the supernatant. The reagent solutions were then drained in syringe-barrel reaction vessels and the resins rinsed five times with alternating acetonitrile and methylene chloride washes. The resins were kept wet with methylene chloride until used in the next step. Cleavage aliquots (40% TFA/CH₂Cl₂) had purities of >80% by HPLC and were registered as PHA compounds (Table 1).

Step 3: Preparation of 10

A stock solution of palladium acetate (0.1 eq., 0.01 mmol, 0.0022 g per 1 mL), triphenylphosphine (0.25 eq., 0.025 mmol, 0.0065 g per 1 mL), and diisopropylethylamine (0.5 eq., 0.05 mmol, 0.0065 g, 0.0087 mL per 1 mL) in 6.5 mL DMF (degassed with N₂) was prepared. To each of the resins (9) in 8 mL vials, 1 mL of stock catalyst solution was added, followed by 0.042 mL of hexamethyl ditin (2.0 eq., 0.2 mmol, 0.065 g). Each vial was purged with nitrogen and then capped. The reaction mixtures were then heated to 60° C. and mixed in an orbital shaker for 17 h. By that time, the resins had all turned black in color. Following cooling, the reaction mixtures were transferred to filter vessels, and reagents were drained. This was followed by washing three times with DMF, three times with alternating acetonitrile/DMF, three times with alternating acetonitrile/methylene chloride, and twice with THF. Cleavage aliquots were taken (cleaved in 40/60 TFA/CH₂Cl₂) to check for completion of reaction by monitoring the protodestannylation products.

Step 4: Preparation of 11

To each of the 8 mL vials holding resins 10, 2 mL of a THF (degassed with carbon monoxide) stock solution containing: 0.0046 g of tris (dibenzylidene acetone)dipalladium (0) (0.05 eq., 0.005 mmol, per 2 mL THF); 0.0052 g of triphenylphosphine (0.2 eq, 0.02 mmol, per 2 mL THF); and 0.139 mL diisopropyl ethylamine (8 eq., 0.80 mmol, 0.103 g, 0.139 mL per 2 mL) were added. Commercially available acid chlorides (8 eq., 0.8 mmol) were then added. The reaction vessels were purged with carbon monoxide, capped and shaken at 60° C. for 18 h. When cool, the reaction mixtures were filtered through ffitted syringe barrels, then the resins rinsed six times with alternating acetonitrile/methylene chloride washes and dried under vacuum at room temperature.

Step 5: Preparation of 1

To each of the fritted vessels containing resins 11, 2 mL of the cleavage cocktail (40/60 TFA/CH₂Cl₂) were added and the mixtures swirled for 45 minutes. Cleavage filtrates were then collected in tared vials followed by stripping of solvents in vacuo. The residues were analyzed by HPLC and ESMS separately. The library was then purified by preparative HPLC. Results for the library both pre- and post-purification are compiled in Table 5.

Preparation of Oximes 13

Ketone precursors to the oxime derivatives were produced as shown above. To 0.1 gram (˜0.12 mmol) of the ketone resins 11 in a 48 well Robbins Block, 2 mL of pyridine were added followed by 10 equivalents (1.2 mmol) of each alkoxyamine (hydroxylamine hydrochloride; methoxyamine hydrochloride; o-benzylohydroxyamine hydrochloride; and o-allylhydroxylamine hydrochloride). The reaction block was sealed and mixed overnight at room temperature in the rotating oven. After 20 hours, the resins resins were drained and washed with 3× (MeOH, CH₂Cl₂) and 3× (MeCN, CH₂Cl₂). Methanol was used early in the wash cycle because MeCN and CH₂Cl₂ left a precipitate in the supernatant at that point. Treatment of the resins with 40% TFA/CH₂Cl₂ for 45 minutes afforded crude products. Four of the library compounds (shown in Table 3) were then successfully purified (>90% pure) via LC/MS.

Amine Derivatives

Preparation of Amines 12

Into four 8 mL vials containing 0.1 grams (˜0.12 mmols) of ketone resin 11, 1.5 mL of toluene along with 0.12 grams (0.42 mmol) of titanium isopropoxide and 2.5 equivalents (0.30 mmol) of each respective amine were added. The vials were purged with nitrogen, sealed with teflon-lined caps, and mixed at room temperature for 16 hours on an orbital shaker. At that time, 0.5 mL of THF, 0.1 mL of acetic acid, and 0.24 grams (1.14 mmol) of sodium triacetoxyborohydride were added and the slurry was mixed at room temperature. After 4 hours, the reagents were drained and the resin washed: 3× (MeOH, DMF), 4× (MeOH, CH₂Cl₂). Treatment of the resin with 40% TFA/CH₂Cl₂ for 45 minutes afforded crude products in the purities included in Table 6. Crude product identities were confirmed by ES/MS.

Step 1: Preparation of 8

To 10.0 grams of Wang resin (frori Unisphere, 1.36 mmol/g loading, 13.6 mmol) in a 250 mL serum bottle, 90 mL of DMF were added followed by 13.4 grams (5 eq., 68 mmol) of 5-chloroisotoic anhydride and 8.3 grams (5 eq., 68 mmol) of 4-dimethylaminopyridine. The serum bottle was purged with nitrogen, capped, and shaken on an orbital mixer at 60° C. Initially, the reagent cocktail was not homogeneous, but after several hours, a concentrated solution had formed around the swelled resin. After 18 hours, the reaction slurry was cooled and transferred to a 60 mL syringe-barrel reaction vessel. The reagent cocktail was then drained and the resin washed as follows: 3× (acetonitrile, DMF), then 3× (acetonitrile, methylene chloride). The resin was treated a second time with 90 mL of DMF, 13.4 grams (5 eq., 68 mmol) of 5-chloroisotoic anhydride, and 13.4 grams (5 eq., 68 mmol) of 4-dimethylaminopyridine. Following mixing at 60° C. for 6 hours, the reagent cocktail was again drained and the resin washed as above. In a vacuum oven at 25° C., the resin was dried for 72 hours to give a final weight of 10.46 grams (1.30 mmol/g loading).

Step 2: Preparation of 9 (R=H)

To 6.2 grams (25 mmol) of the meta- and para-iodo benzoic acids suspended in 70 mL of methylene chloride, 40 μL of DMF and 4.4 mL (6.35 grams, 50 mmol, 2 eq.) of oxalyl chloride were added. The serum bottles were sealed and stirred with occasional release of gas build-up. After stirring for 5 hours, the reaction mixtures had become almost completely homogeneous with no more gas build-up. Solvent and excess oxalyl chloride were then evaporated in vacuo to dryness. The acid chlorides were re-dissolved in 30 mL of methylene chloride and added to 4 gram of resin 8 (1.30 mmol/gram loading, 5.2 mmol) swollen with 30 mL of pyridine in 125 mL serum bottles. Some fuming was observed initially. The mixtures were purged with nitrogen for 10 seconds then the vials capped, and the mixtures shaken at room temperature for 4 hours. By that time, the resins had taken on a light orange color and a tan precipitate had formed in the supernatant. The reagent solutions were then drained in syringe-barrel reaction vessels and the resins rinsed five times with alternating acetonitrile and methylene chloride washes. The resins were then dried in vacuo to afford 5.14 g of the meta-iodo product and 5.09 g of the para-iodo product. Cleavage aliquots were >95 % pure by HPLC, with their identities confirmed by ESMS.

Step 3: Preparation of 10 (R═H)

A stock solution of palladium acetate (0.012 M), triphenylphosphine (0.03 M), and diisopropylethylamine (0.06 M) in 80 mL DMF (degassed with N₂) was prepared. To 4.0 grams (˜5.0 mmol) of each resin (9) in 125 mL serum bottles, 40 mL of the stock catalyst solution were added, followed by 2.0 mL of hexamethyl ditin (2.0 eq., 9.6 mmol, 3.14 g). Each bottle was purged with nitrogen and then capped. The reaction mixtures were then heated to 60° C. and mixed in an orbital shaker for 17 h. By that time, the two resins had turned black in color. Following cooling, the reaction mixtures were transferred to filter vessels, and reagents were drained. This was followed by washing three times with DMF, three times with alternating acetonitrile/DMF, three times with alternating acetonitrile/methylene chloride, and twice with THF. Cleavage aliquots were taken (cleaved in 40/60 TFA/CH₂Cl₂) to check for completion of reaction by monitoring the protodestannylation products. Following cleavage, the meta-substituted resin gave 87% of the expected destannylated product by HPLC, while the para-substituted isomer gave 70%. Little to no iodide starting material remained. The major impurity in both cases was an unidentified peak with [M+H]⁺=369 m/z.

Step 4: Preparation of 11

To each carboxylic acid weighed into a 20 mL vial (2.88 mmol), 6.5 mL of THF, 10 μL of DMF, and 0.293 ml of oxalyl chloride (0.95 eq., 2.7 mmol, 3.35 g) were added. The vials were sealed and reaction mixtures shaken at room temperature for 4 hours with occasional release of evolved gas. In the meantime, the two stannylated resins (10) were distributed into Irori minikans (60 mg per kan), and the 72 kans were then distributed into twelve 125 mL serum bottles (six kans per bottle). To each of the bottles, 20 mL of a nitrogen degassed THF stock solution containing: tris (dibenzylidene acetone)dipalladium (0) (0.001 M); potassium carbonate (0.02 M); and diisopropyl ethylamine (0.10 M) were added. The THF solutions of acid chlorides (2.88 mmol, 6 eq.) were then added to their respective set of six bottles. The capped reaction vessels were purged with nitrogen, degassed, and shaken at 65° C. for 18 h. When cool, the resin containing kans were rinsed five times with alternating acetonitrile/methylene chloride washes and dried under vacuum at room temperature. A cleavage aliquot revealed that ketone formation had gone to completion.

Step 5a: Preparation of Resin-Bound Amines 12

To a 125 mL serum bottle containing 24 Irori cans loaded with resin 11, 30 mL of toluene were added, followed by 1.23 grams (6.0 mmol, 3.5 eq.) of titanium isopropoxide and 0.25 grams (4.3 mmol, 2.5 eq.) of propyl amine. The bottle was degassed to remove air bubbles from the Irori kans, then purged with nitrogen, sealed and mixed for 17 hours at room temperature. At that time, 10 mL of toluene, 2 mL of acetic acid, and 3.5 grams (16.3 mmol, 9.5 eq.) of sodium triacetoxy borohydride were added, and bottle re-purged and sealed, and mixed for 14 hours. Reagents were then drained and the resins washed three times with methanol and five times with alternating methanol/methylene chloride.

Step 5b: Preparation of Resin-Bound Oximes 13

To a 125 mL serum bottle containing 24 Irori kans loaded with resin 11, 40 mL of pyridine were added followed by 1.2 grams (17.2 mmols, 10 eq.) of hydroxylamine hydro chloride. The bottle was degassed to remove air bubbles from the Irori kans, then purged with nitrogen, sealed and mixed for 17 hours at room temperature. At that time, reagents were drained and the resins were washed three times with methanol, and five times with alternating methanol/methylene chloride.

Step 6: Preparation of 15

The 72 kans containing resins 12,13 were distributed into tared 8 mL vials and treated with 3 mL of TFA/CH₂Cl₂ (40/60). The vials were degassed, capped, and mixed at room temperature for 1.5 hours. The kans were then plucked out of the vials using a syringe needle and washed with another 1 mL of CH₂Cl₂. Solvent in the vials was evaporated in vacuo (Genevac), leaving product residue.

### Preparation of 5-Iodoisatoic Anhydride

To a red-brown solution of 2-amino-5-iodobenzoic acid (25 grams, 95 mmol) in 300 mL of dioxane, 9.58 grams (32.3 mmol) of triphosgene were carefully added. The resulting slurry was refluxed for 4 hours. By that time, all starting material had disappeared by HPLC. The solid product was then filtered, washed once with ethyl ether, then dried overnight in a vacuum oven at 40° C. The tan colored needles amounted to 22.9 grams (83%). HPLC (MRH1 method): t_{R}=2.15 min. (100%); ¹H NMR (400 MHz, DMSO-d₆) δ 8.12 (s, 1 H), 8.00 (d, J=8.6 Hz, 1 H), 6.95 (d, J=8.5 Hz, 1 H); MS (ES) m/z (rel. intensity) 288 (M—, 100), 244 (5), 289 (5); 577 (10).

### Preparation of 6-Chloroindoline

In a 250 mL round bottom flask, 12.4 grams of sodium cyanoborohydride (198 mmol, 2 eq.) were added potion-wise over 5 minutes to a solution of 15 grams (98.9 mmol) of 6-chloroindole. After stirring for 22 hours, the mixture had become a brown solution and analysis by HPLC (MRH 1 method) revealed no starting material remaining and a mixture of two product peaks. The mixture was diluted with 100 ml of water, then made basic with ˜200 mL of 6N sodium hydroxide. The desired product was extracted into 3×400 mL of methylene chloride. The extracts were then dried over anhydrous magnesium sulfate and evaporated in vacuo leaving a cloudy oil. The crude product was chromatographed over a plug of silica in 100% methylene chloride giving a mixed fraction (Rf=0.9 and 0.7), a pure product fraction (Rf=0.7), and a baseline fraction (Rf=0.0-0.2). The pure fraction was evaporated to dryness in vacuo to yield a clear, colorless oil weighing 10.90 grams (72%). It was stored at 4° C. and saved for future use. ¹H NMR (300 MHz, DMSO-d₆) δ 6.95 (d, J=5 Hz, 1 H), 6.46 (d, J=5 Hz, 2 H), 3.43 (t, J=6, 2 H), 2.86 (t, J=6, 2 H).

### Example 4

### Amide Derivatives

Standard procedure for attaching 5-bromoanthranilc acid to hydroxymethyl styrene resin:

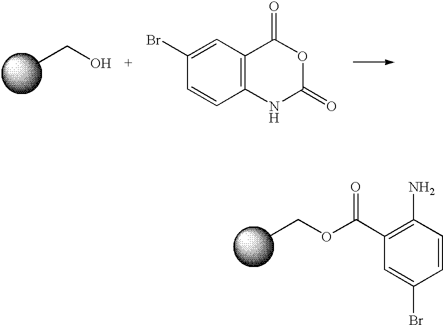

To a slurry of 24.8 g (36.7 mmol) hydroxymethyl styrene resin in 1 L DMF was added 24 g (197 mmol) 4-dimethylamino pyridine and 50 g (207 mmol) 5-bromoisatocanhydride. The mixture was stirred at 60° C. for 18 hours and room temperature for four hours. The mixture was then filtered and the resin washed repeatedly alternating with dichloromethane and DMF (3×) then repeatedly alternating with dichloromethane and methanol (3×) followed by methanol (3×). The resin was dried over night in a vacuum oven.

Resin 2 and 3:

Standard procedure for attaching 3 or 4-N-boc-amino benzoic acid to resin 1.

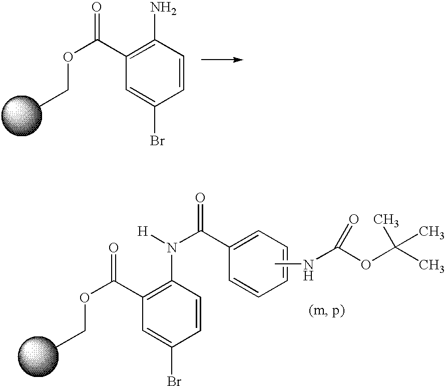

To 5.1 g (21.5 mmol) 3-N-boc-aminobenzoic acid in 200 mL of anhydrous THF was added 100 μL DMF and 2.3 mL (25.8 mmol) oxalyl chloride in five portions over 20 minutes. After 40 minutes the mixture was concentrated in vacuo and then dissolved in 50 mL dichloromethane. This was added to a slurry of 3.79 g (4.32 mmol) resin 1 in 150 mL dichloromethane and 3.7 mL diisopropylethyl amine. The mixture was heated to reflux over night. The resin was then collected by vacuum filtration and washed repeatedly alternating with dichloromethane and methanol (4×) followed by methanol (3×) and dried in a vacuum oven. The same procedure was followed to prepare resin 3 from 4-N-boc-aminobenzoic acid.

Standard procedure for the acylation of resins 2 and 3 with acid chlorides, isocyanates, and isothiocyanates.

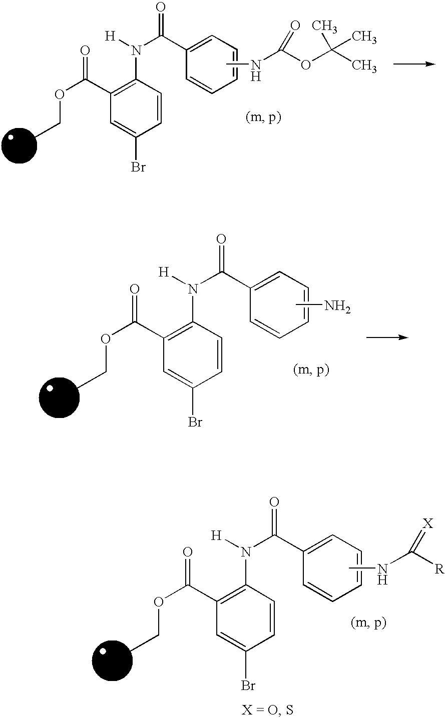

On average 55 mg (Ca. 0.055 mmol) resin was treated with 33% TFA in DCM for two hours. The resin was collected by filtration and washed repeatedly alternating with dichloromethane and methanol (4×) followed by methanol (3×) and dried in a vacuum oven. The resin is then treated with 0.6 mmol of the acylating reagent and 0.86 mmol diisopropylethyl amine in DCM and shaken over night. The resin was then collected by vacuum filtration and washed repeatedly alternating with dichloromethane and methanol (4×) followed by methanol (3×) and dried in a vacuum oven

Standard procedure for the acylation of resins 2 and 3 with sulfonyl chlorides:

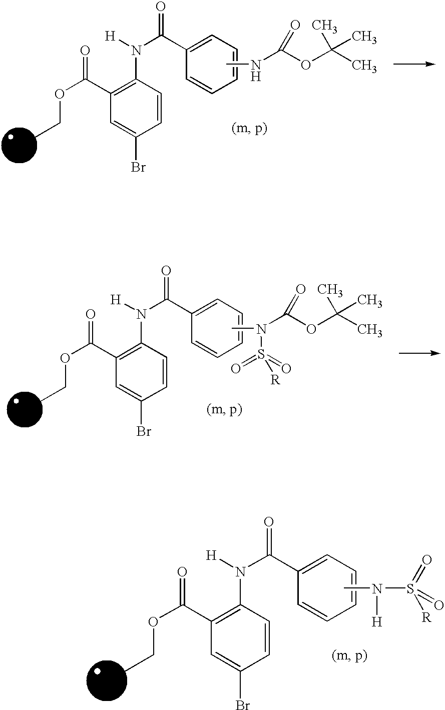

On average, to 60 mg (Ca. 0.06 mmol) resin in 2 mL DCM was added 10 equivalents of a sulfonyl chloride and 174 μL (0.6 mmol) 2-tert-butylimino-2-diethyl-amino-1,3-diemthylperhydro-1,3,2-diazaphosphorine (BEMP). After mixing overnight, the resin was collected by vacuum filtration and washed repeatedly alternating with dichloromethane and methanol (4×) followed by methanol (3×) and dried in a vacuum oven. The resin was then treated with 2 mL of 40% TFA in DCM for one hour and then collected by vacuum filtration and washed repeatedly alternating with dichloromethane and methanol (4×) followed by methanol (3×) and dried in a vacuum oven.

Standard cleavage procedure to provide products.

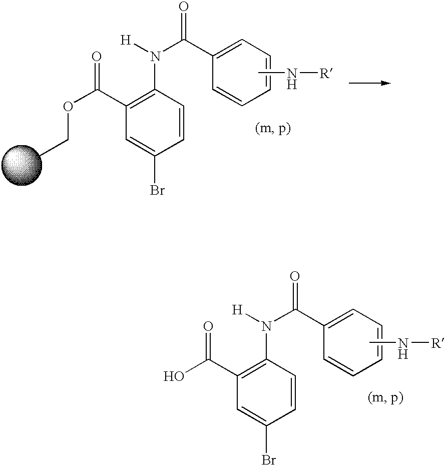

The resin was treated with 1.5 mL THF and 0.5 mL 1 N sodium hydroxide over night. The mixtures were filtered and the collected filtrate was treated with 250 mg of IR-120 acidic resin for 2.5 hours. The mixtures were filtered and the filtrates concentrated to provide the following products. If initial purity was less than 80% by HPLC those products were purified by chromatography.

Several compounds were produced by the above-described methodologies.
2-{[3-(benzoylamino)benzoyl]amino}-5-bromobenzoic acid
5-bromo-2-{[3-(2-furoylamino)benzoyl]amino}benzoic acid
5-bromo-2-({3-[(thien-2-ylacetyl)amino]benzoyl}amino)benzoic acid
5-bromo-2-({3-[(mesitylcarbonyl)amino]benzoyl}amino)benzoic acid
5-bromo-2-({4-[(mesitylcarbonyl)amino]benzoyl}amino)benzoic acid
2-({3-[(1,3-benzodioxol-5-ylcarbonyl)amino]benzoyl}amino)-5-bromobenzoic acid
5-bromo-2-({3-[(2,4-dimethoxybenzoyl)amino]benzoyl}amino)benzoic acid
5-bromo-2-[(3-{[(phenylthio)acetyl]amino}benzoyl)amino]benzoic acid
5-bromo-2-({3-[(methoxyacetyl)amino]benzoyl}amino)benzoic acid
2-({3-[(anilinocarbonyl)amino]benzoyl}amino)-5-bromobenzoic acid
5-bromo-2-{[3-({[(2,4-difluorophenyl)amino]carbonyl}amino)benzoyl]amino}benzoic acid
5-bromo-2-{[3-({[(3-cyanophenyl)amino]carbonyl}amino)benzoyl]amino}benzoic acid
5-bromo-2-{[3-({[(3-chlorophenyl)amino]carbonyl }amino)benzoyl]amino}benzoic acid
5-bromo-2-({3-[({[3-(methylthio)phenyl]amino}carbonyl)amino]benzoyl}amino)benzoic acid
2-{[3-({[(3-acetylphenyl)amino]carbonyl}amino)benzoyl]amino}-5-bromobenzoic acid
5-bromo-2-({4-[(phenylsulfonyl)amino]benzoyl}amino)benzoic acid
5-bromo-2-{[3-({[4-(trifluoromethoxy)phenyl]sulfonyl}amino)benzoyl]amino}benzoic acid
5-bromo-2-{[4-({[4-(trifluoromethoxy)phenyl]sulfonyl}amino)benzoyl]amino}benzoic acid
5-bromo-2-[(4-{[(3,4-dichlorophenyl)sulfonyl]amino}benzoyl)amino]benzoic acid
5-bromo-2-({4-[(thien-2-ylacetyl)amino]benzoyl}amino)benzoic acid
5-bromo-2-({3-[(5-nitro-2-furoyl)amino]benzoyl}amino)benzoic acid
5-bromo-2-({4-[(5-nitro-2-furoyl)amino]benzoyl}amino)benzoic acid
5-bromo-2-{[4-({[(2,4-difluorophenyl)amino]carbonyl}amino)benzoyl]amino}benzoic acid
5-bromo-2-{[3-({[(3,5-dichlorophenyl)amino]carbonyl}amino)benzoyl]amino}benzoic acid
5-bromo-2-{[3-({[(5-chloro-2-methoxyphenyl)amino]carbonyl}amino)benzoyl]amino}benzoic acid
5-bromo-2-{[3-({[(4-phenoxyphenyl)amino]carbonyl3amino)benzoyl]amino}benzoic acid
5-bromo-2-{[4-({[(4-phenoxyphenyl)amino]carbonyl}amino)benzoyl]amino}benzoic acid
2-{[3-({[(4-acetylphenyl)amino]carbonyl}amino)benzoyl]amino}-5-bromobenzoic acid
5-bromo-2-{[4-({[(4-nitrophenyl)amino]carbonothioyl}amino)benzoyl]amino}benzoic acid
5-bromo-2-({3-[({[2-(trifluoromethyl)phenyl]amino}carbonothioyl)amino]benzoyl}amino)benzoic acid
5-bromo-2-{[3-({[(3,4,5-trimethoxyphenyl)amino]carbonothioyl}amino)benzoyl]amino}benzoic acid
5-bromo-2-({3-[({[3-(methylthio)phenyl]amino}carbonothioyl)amino]benzoyl}amino)benzoic acid
2-{[3-({[(3-acetylphenyl)amino]carbonothioyl}amino)benzoyl]amino}-5-bromobenzoic acid
5-bromo-2-({3-[(phenylsulfonyl)amino]benzoyl}amino)benzoic acid
5-bromo-2-[(3-{[(3,4-dichlorophenyl)sulfonyl]amino}benzoyl)amnino]benzoic acid
5-bromo-2-[(4-{[(4-methylphenyl)sulfonyl]amino}benzoyl)amino]benzoic acid

Analogs with an alternative linkage, such as ureas, in place of the sulfonamides described in Example 1 were also synthesized.

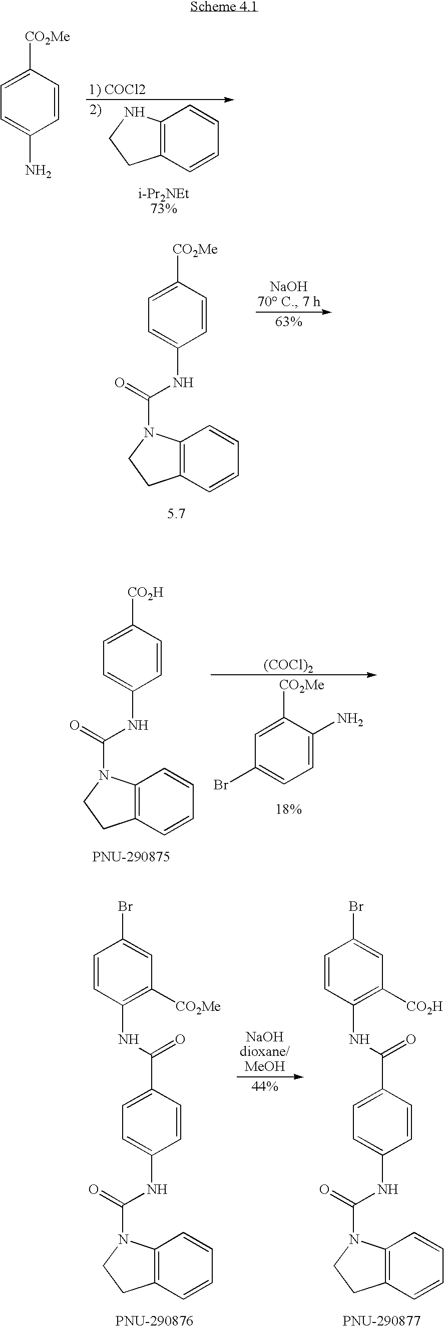

### Methyl 4-[(2,3-dihydro-1H-indol-1-ylcarbonyl)amino]benzoate

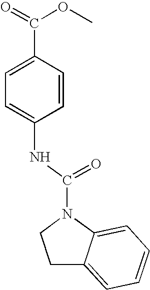

Methyl-4-aminobenzoate (100 g, 7.29 mmol) in DCM (50 mL) was slowly added to a solution of phosgene (1.93 M /toluene, 7.5 mL, 14.5 mol, 2.0 equiv) in DCM (200 mL) at 0° C., follwed by the addition of diisopropylethyl amine (1.14 mL, 6.56 mmol, 0.9 equiv). The mixture was allowed to warm to rt, then stirred for 1 h, and then concentrated in vacuo to ca 5 mL. The suspension was redissolved in DCM followed by the addition of indoline (2.45 mL, 21.87 mmol, 3.0 equiv) and diisopropylethyl amine (1.14 mL, 6.56 mmol, 0.9 equiv). The resulting mixture was stirred for 2 h, at rt, then washed with 1N HCl, brine, dried (MgSO₄) filtered and concentrated in vacuo. The residue was recrystallized from EtOH to afford 1.67 g of 5.7 as a white solid.

¹H NMR (300 MHz, CDCl₃) δ 8.04-8.01 (m, 2 H), 7.90 (d, J=7.9 Hz, 1 H), 7.58-7.55 (m, 2 H), 7.28-7.20 (m, 2 H), 7.01 (t, J=8.2 Hz, 1 H), 6.70 (s, 1 H), 4.12 (t, J=8.3 Hz, 2 H), 3.91 (s, 3 H), 3.26 (t, J=8.2 Hz, 2 H).

### 4-[(2,3-Dihydro-1H-indol-1-ylcarbonyl)amino]benzoic acid

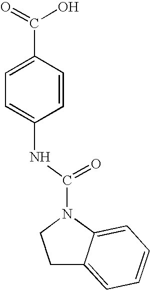

Methyl 4-[(2,3-dihydro-1H-indol-1-ylcarbonyl)amino]benzoate (1.30 g, 4.37 mmol) was placed in dioxane (50 mL) with 5 N NaOH (10 mL) and the resulting solution was heated at 70° C. for 7 h. The reaction was cooled to rt, acidified, diluted with EtOAc and washed with H₂O, brine, dried (MgSO₄) filtered and concentrated in vacuo. The residue was recrystallized from EtOH to afford 776 mg (63%) of a white solid.

¹H NMR (300 MHz, DMSO-d₆) δ 8.82 (s, 1 H), 7.87 (d, J=8.6 Hz, 3 H), 7.71 (d, J=8.7 Hz, 2 H), 7.22-7.14 (m, 2 H), 6.92 (t, J=7.3 Hz, 1 H), 4.16 (t, J=8.4 Hz, 2 H), 3.18 (t, J=8.5 Hz, 2 H).

### 5-Bromo-2-({4-[(2,3-dihydro-1H-indol-1-ylcarbonyl)amino]benzoyl}amino)benzoic acid, PNU-290877

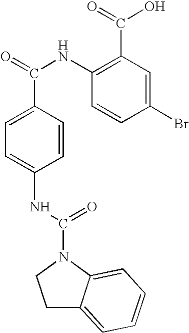

4-[(2,3-dihydro-1H-indol-1-ylcarbonyl)amino]benzoic acid (627 mg, 2.22 mmol) was dissolved in DCM (30 mL) follwed by the addition of oxalyl chloride (490 μL, 5.55 mmol, 2.5 equiv) and DMF (30 μL). The mixture was stirred for 1 h, then diluted with heptane (10 mL), concentrated in vacuo to dryness. The residue was redissolved in DCM (50 mL) followed by the addition of methyl-2-amino-5-bromo benzoate (510 mg, 2.2 mmol, 1.0 equiv.) and pyridine (360 μL, 4.4 mmol, 2.0 equiv.) The reaction was stirred for 3 h at rt, then washed with 1 N HCl, 1 N NaOH, H₂O, brine, dried (MgSO₄) filtered and concentrated in vacuo. The residue was purified by silica gel chromatography (heptane/EtOAc 19/1, 9/1, 4/1, 1/1, 0/1) to afford 198 mg (18%) of a white solid as the methyl ester. The ester (177 mg, 0.35 mmol) was dissolved in dioxane (10 mL) follwed by the addition of 5 N NaOH (5 mL). The reaction was stirred for 3 h at rt, diluted with EtOAc, washed with 1 N HCl, brine, dried (MgSO₄), filtered and concentrated in vacuo. The residue was recrystallized from EtOH to afford 76 mg (44%) of a white solid.

¹H NMR (300 MHz, DMSO-d₆) δ 8.88 (s, 1 H), 8.69 (d, J=9.0 Hz, 1 H), 8.13 (d, J=2.4 Hz, 1 H), 7.86-7.78 (m, 6 H), 7.21-7.14 (m, 2 H), 6.93 (t, J=8.6 Hz, 1 H), 4.17 (t, J=8.2 Hz, 2 H), 3.19 (t, J=8.2 Hz, 1 H).

### Example 5

### Alkyl Derivatives

### Preparation of 3-(Phenylethynyl)benzoic acid

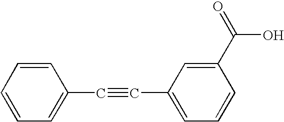

A flask containing ethyl 3-iodobenzoate (2.21 g, 8.00 mmol, Lancaster), copper (I) iodide (550 mg, 2.88 mmol, Alfa), and tetrabutylammonium iodide (5.9 g, 16 mmol, Aldrich) was placed under argon. DMF (40 mL), diisopropylethylamine (4.5 mL, 26 mmol, Aldrich), and tri-t-butylphosphine (1.8 g of 10 wt % solution in hexane, 0.89 mmol, Strem) were added by syringe. Tris(dibenzylideneacetone)dipalladium(0)-chloroform adduct (220 mg, 0.21 mmol, Aldrich) was added as a solid under a flow or argon. The mixture was stirred for 5 minutes, and phenylacetylene (0.88 mL, 8.0 mmol, Lancaster) was added by syringe. After 40 minutes, the mixture was added to a separatory funnel with 200 mL of saturated aqueous NaHCO₃. Product was extracted into 3×100 mL of EtOAc. The combined EtOAc was washed with 4×200 mL of water and then dried over MgSO₄. Product was adsorbed onto silica and purified by chromatography using a Biotage Flash 40 M silica cartridge with a gradient from 25%-40% CH₂Cl₂ in heptane. The ethyl 3-(phenylethynyl)benzoate was isolated as 1.82 g of brown oil that was contaminated with tri-t-butylphosphine. 990 mg of this oil was dissolved in dioxane (15 mL) and treated with 1 M aqueous sodium hydroxide (6 mL), and the mixture was stirred for 3.5 hours. It was then added to a separatory funnel with 100 mL of 1 M aqueous HCl and 100 mL of CH₂Cl₂. A few milliliters of THF were added to help with solubility. The organics were washed with an additional 100 mL of HCl followed by 100 mL of water and then dried over MgSO₄. Solvent was removed leaving 782 mg of tan solid that was still contaminated with phosphine. Most of this material was carried on without further purification. For the purposes of characterization, the remainder was recrystallized from ethanol/heptane yielding a white solid.

### 5-Bromo-2-{[3-(phenylethynyl)benzoyl]amino}benzoic acid

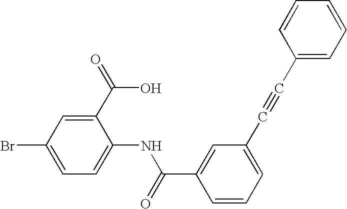

To 3-(phenylethynyl)benzoic acid (569 mg, 2.56 mmol) in CH₂Cl₂ (20 mL) was added DMF (40 82 L) and oxalyl chloride (450 μL, 5.16 mmol). The mixture was stirred for 2.5 hours, and the solvent and excess oxalyl chloride were removed by rotary evaporation. The residue was dissolved in CH₂Cl₂ (15 mL), and methyl 2-amino-5-bromobenzoate (504 mg, 2.19 mmol, Avocado) in pyridine (6 mL) was added. The mixture was stirred overnight and then added to a separatory funnel with 100 mL of CH₂Cl₂. This solution was washed with 2×100 mL of 1 M aqueous HCl and 100 mL of brine. The CH₂Cl₂ was evaporated in the presence of silica gel, and the product was purified by chromatography using a Biotage Flash 40 M silica cartridge with a gradient from 50% -60% CH₂Cl₂ in heptane as eluent. Yield was 694 mg of white solid as the methyl ester. To a mixture of the methyl ester (485 mg, 1.12 mmol) in dioxane (15 mL) was added 1 M aqueous sodium hydroxide (2.2 mL). The mixture was stirred for 2.75 hours. The reaction mixture was added to a separatory finnel with 100 mL of 1 M aqueous HCl, and the product was extracted into 100 mL of CH₂Cl₂. The CH₂Cl₂ was washed with an additional 100 mL of 1 M aqueous HCl followed by 100 mL of water. It was then dried over MgSO₄ and evaporated. The residue was recrystallized from hot ethanol/THF. The solids were washed with ethanol followed by heptane and then dried at 100° C. under vacuum yielding 295 mg of white solid. ¹H NMR (400 MHz, DMSO-d₆) δ 12.06 (s, 1 H), 8.60 (d, J=9.2 Hz, 1 H), 8.12 (d, J=2.0 Hz, 1 H), 8.10 (s, 1 H), 7.97 (d, J=7.6 Hz, 1 H), 7.87 (dd, J=9.2, 2.5 Hz, 1 H), 7.83 (d, J=8.1 Hz, 1 H), 7.66 (t, J=7.6 Hz, 1 H), 7.59-7.63 (m, 2 H), 7.45-7.48 (m, 3 H).

### Preparation of 3-(2-Phenylethyl)benzoic acid

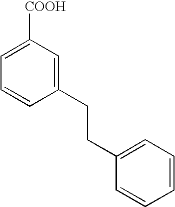

A mixture of 3-(phenylethynyl)benzoic acid (418 mg, 1.88 mmol) and palladium on carbon (315 mg, 10%, Aldrich) in 1:1 methanol/THF (20 mL) was stirred under 1 ATM of hydrogen overnight. The mixture was then filtered through a plug of celite and concentrated yielding 406 mg of white solid. This material was carried forward without further purification. For the purposes of characterization, a small amount of the product was recrystallized from toluene.

### 5-Bromo-2-{[3-(2-phenylethyl)benzoyl]amino}benzoic acid

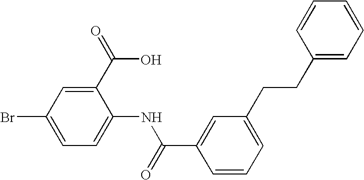

To 3-(2-phenylethyl)benzoic acid (292 mg, 1.29 mmol) in CH₂Cl₂ (20 mL) was added DMF (20 μL) and oxalyl chloride (225 μL, 2.58 mmol). The mixture was stirred for 2.5 hours, and the solvent and excess oxalyl chloride were removed by rotary evaporation. The residue was dissolved in CH₂Cl₂ (10 mL), and methyl 2-amino-5-bromobenzoate (248 mg, 1.08 mmol, Avocado) in pyridine (4 mL) was added. The mixture was stirred overnight and then added to a separatory funnel with 100 mL of CH₂Cl₂. This solution was washed with 2×100 mL of 1 M aqueous HCl and 100 mL of brine. The CH₂Cl₂ was evaporated in the presence of silica gel, and the product was purified by chromatography using a Biotage Flash 40 M silica cartridge with a gradient from 50% -100% CH₂Cl₂ in heptane as eluent. Yield was 361 mg of white solid as the methyl ester. To a mixture of the methyl ester (285 mg, 0.65 mmol) in dioxane (10 mL) was added 1 M aqueous sodium hydroxide (1.0 mL). The mixture was stirred at room temperature for 1 hour and then heated in a 50° C. oil bath for 15 minutes. The reaction mixture was added to a separatory funnel with 100 mL of 1 M aqueous HCl, and the product was extracted into 100 mL of CH₂Cl₂. The CH₂Cl₂ was washed with an additional 100 mL of 1 M aqueous HCl followed by 100 mL of water. It was then dried over MgSO₄ and evaporated. The residue was recrystallized from hot ethanol. The solids were washed with heptane and then dried at 100° C. under vacuum yielding 88 mg of white solid. ¹H NMR (400 MHz, DMSO-d₆) δ 12.10 (s, 1 H), 8.68 (d, J=9.1 Hz, 1 H), 8.12 (d, J=2.5 Hz, 1 H), 7.83-7.87 (m, 2 H), 7.75-7.78 (m, 1 H), 7.46-7.51 (m, 2 H), 7.16-7.31 (m, 5 H), 2.91-3.02 (m, 4 H).

### Example 6

Thioamide Linkers.

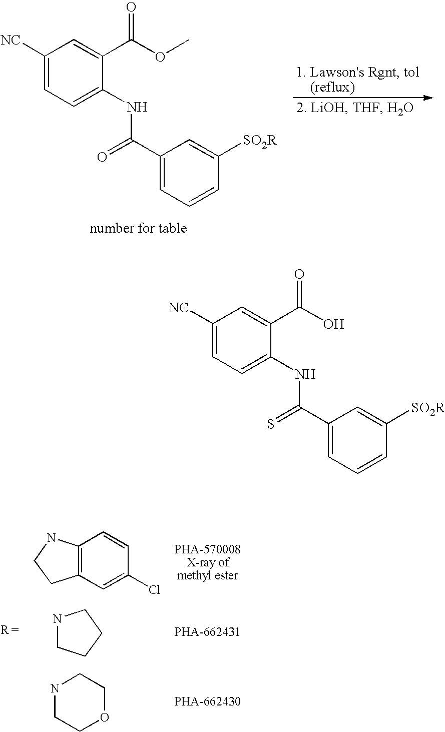

### 2-[({3-[(5-Chloro-2,3-dihydro-1H-indol-1-yl)sulfonyl]phenyl}carbonothioyl)amino]-5-cyanobenzoic acid

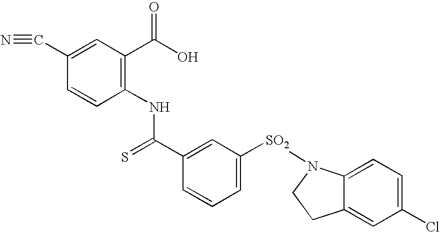

General procedure A: Methyl 2-[({3-[(5-chloro-2,3-dihydro-1H-indol-1-yl)sulfonyl]phenyl}carbonyl)amino]-5-cyanobenzoate (989 mg, 1.99 mmol) and Lawesson's reagent (4.5 g, 11.1 mmol) were combined in a flask equipped with a reflux condensor. The flask was evacuated and purged with N₂ several times. Tol (30 mL) was added and the reaction was refluxed overnight. The reaction was cooled to rt and filtered to remove excess Lawesson's reagent. The filtrate was absorbed in SiO₂ and the product was purified by silica gel chromatography using Hept/EtOac (19:1, 9:1, 3:17, 4:1). The product was triturated with MeOH to afford 670 mg (66%) of an orange solid as the methyl ester. ¹H NMR (DMSO-d₆) δ 12.40 (s, 1 H), 8.35 (d, J=2 Hz, 1 H), 8.29 (s, 1 H), 8.19 (dd, J=8, 2 Hz, 1 H), 8.14 (d, J=8 Hz, 1 H), 8.06 (d, J=8 Hz, 1 H), 7.98 (d, J=8 Hz, 1 H), 7.73 (t, J=8 Hz, 1 H), 7.49 (d, J=9 Hz, 1 H), 7.30-7.25 (m, 2 H), 4.02 (t, J=8 Hz, 2 H), 3.79 (s, 3 H), 2.97 (t, J=8 Hz, 2 H).

General procedure B: to a solution of the methyl ester (300 mg, 0.605 mmol) dissolved in THF (7 mL) and H₂O (1.5 mL) was added LiOH—H₂O (450 mg, 10.7 mmol) and the reaction was heated to 45° C. for 6 hr. The solution was diluted with MTBE, washed with 2 N HCl and brine, dried (MgSO₄), concentrated, and triturated with MeOH to afford 252 mg (84%) of an orange solid. ¹H NMR (DMSO-d₆) δ 8.62 (d, J=8 Hz, 1 H), 8.36 (dd, J=12, 2 Hz, 1 H), 8.18 (d, J=8 Hz, 1 H), 8.12 (dd, J=8, 2 Hz, 1 H), 7.95 (d, J=8 Hz, 1 H), 7.71 (t, J=8 Hz, 1 H), 7.48 (d, J=9 Hz, 1 H), 7.27-7.25 (m, 2 H), 4.02 (t, J=8 Hz, 2 H), 2.96 (t, J=8 Hz, 2 H).

### Methyl 2-{[3-(chlorosulfonyl)benzoyl]amino}-5-cyanobenzoate

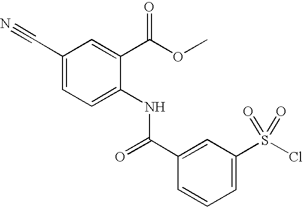

To a suspension of 3-(chlorosulfonyl)benzoic acid (10.8 g, 49.0 mmol) in CH₂Cl₂ (105 mL) and three drops of DMF was added oxalyl chloride (12.5 mL) and the reaction was stirred at rt overnight. The solution was concentrated in vacuo, diluted with CH₂Cl₂ (100 mL), and the solution was divided into two reactions. A 50 mL (24.5 mmol) aliquot of the acid chloride was added to a solution of PHA-522499 (4.49 g, 25.5 mmol) dissolved in CH₂Cl₂ (50 mL) and pyridine (3.0 mL) and stirred at rt overnight. The solution was diluted with MTBE, washed with 2 N HCl and brine, concentrated, triturated with MTBE to afford 7.91 g (85%) of methyl 2-{[3-(chlorosulfonyl)benzoyl]amino}-5-cyanobenzoate as a tan solid. ¹H NMR (400 MHz, DMSO-d₆) δ 11.73 (s, 1 H), 8.67 (d, J=9 Hz, 1 H), 8.37 (d, J=2 Hz, 1 H), 8.25 (s, 1 H), 8.12 (dd, J=9, 2 Hz, 1 H), 7.92 (d, J=8 Hz, 1 H), 7.88 (d, J=8 Hz, 1 H), 7.60 (t, J=8 Hz, 1 H), 3.93 (s, 3 H).

### 5-cyano-2-{[3-(pyrrolidin-1-ylsulfonyl)benzoyl]amino}benzoic acid

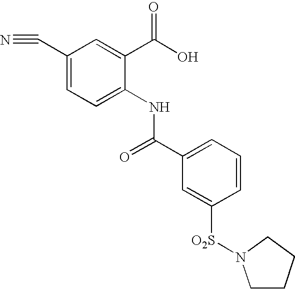

General procedure C: To a solution of methyl 2-{[3-(chlorosulfonyl)benzoyl]amino}-5-cyanobenzoate (1.863 g, 4.92 mmol) dissolved in CH₂Cl₂ (40 mL) was added pyrrolidine (1.5 mL, 18.0 mmol) and stirred at rt for 3 hr. The reaction was diluted with MTBE, washed with 2 N HCl and brine, concentrated, and triturated with MeOH to afford 1.70 g (84%) of methyl 5-cyano-2-{[3-(pyrrolidin-1-ylsulfonyl)benzoyl]amino}benzoate as a tan solid. ¹H NMR (300 MHz, DMSO-d₆) δ 11.75 (s, 1 H), 8.61 (d, J=9 Hz, 1 H), 8.38 (d, J=2 Hz, 1 H), 8.33 (s, 1 H), 8.25 (d, J=8 Hz, 1 H), 8.14 (dd, J=9, 2 Hz, 1 H), 8.10 (d, J=8 Hz, 1 H), 7.90 (t, J=8 Hz, 1 H), 3.91 (s, 3 H), 3.24-3.19 (m, 4 H), 1.71-1.66 (m, 4 H) Methyl 2-{[3-(chlorosulfonyl)benzoyl]amino}-5-cyanobenzoate (378 mg, 1.0 mmol) was dissolved in 15 mL of CHCl₃. Pyrrolidine (145 mg, 2.0 mmol) and Et₃N (1 mL) were then added and the reaction stirred at room temperature for 12 hr. The mixture was poured into 1 M HCl (20 mL) and extracted with EtOAc (3×20 mL). The combined organic solutions were dried over Na₂SO₄ and concentrated in vacuo. The resulting residue was purified by silica gel chromatography, providing 297 mg (72%) of the desired methyl ester. The ester was treated with LiOH in 1:1:1 THF/MeOH/H₂O for 12 hrs followed by acidification and extraction with EtOAc. The organic solution was dried over Na₂SO₄ and then concentrated in vacuo. The title compound (249 mg, 87%) was obtained as a white solid after recrystalization from MeOH. H NMR (300 MHz, DMSO) 1.67 (m, 4H), 3.20 (m, 4H), 7.88 (t, 1H), 8.09-8.14 (m, 2H), 8.26 (d, 1H), 8.33 (s, 1H), 8.42 (d, 1H), 8.83 (d, 1H), 12.56 (s, 1H)

### 5-Cyano-2-({[3-(pyrrolidin-1-ylsulfonyl)phenyl]carbonothioyl}amino)benzoic acid

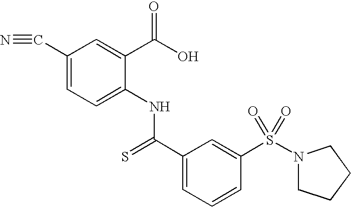

Prepared according to general procedure A: Methyl 5-cyano-2-{[3-(pyrrolidin-1-ylsulfonyl)benzoyl]amino}benzoate (1.12 g, 2.70 mmol) and Lawesson's reagent (5.5 g, 13.6 mmol) afforded 450 mg of a mixture of the methyl ester and Lawesson's reagent after puriflying by silica gel chromatography twice. The crude material was hydrolyzed according to general method B to afford 253 mg (29%) over two steps of an orange solid. ¹H NMR (300 MHz, DMSO-d₆) δ 9.80 (d, J=9 Hz, 1 H), 8.42 (d, J=2 Hz, 1 H), 8.33 (s, 1 H), 8.23 (d, J=8 Hz, 1 H), 7.97-7.91 (m, 2 H), 7.75 (t, J=7 Hz, 1 H), 3.23-3.19 (m, 4 H), 1.71-1.65 (m, 4 H).

### 5-cyano-2-{[3-(morpholin-4-ylsulfonyl)benzoyl]amino}benzoic acid

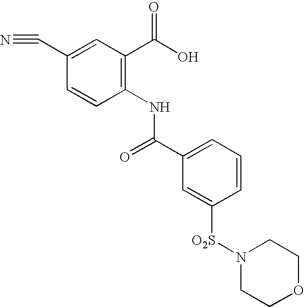

Methyl 2-{[3-(chlorosulfonyl)benzoyl]amino}-5-cyanobenzoate (378 mg, 1.0 mmol) was dissolved in 15 mL of CHCl₃. Morpholine (156 mg, 2.0 mmol) and Et₃N (1 mL) were then added and the reaction stirred at room temperature for 12 hr. The mixture was poured into 1 M HCl (20 mL) and extracted with EtOAc (3×20 mL). The combined organic solutions were dried over Na₂SO₄ and concentrated in vacuo. The resulting residue was purified by silica gel chromatography, providing 373 mg (87%) of the desired methyl ester. The ester was treated with LiOH in 1:1:1 THF/MeOH/H₂O for 12 hrs followed by acidification and extraction with EtOAc. The organic solution was dried over Na₂SO₄ and then concentrated in vacuo. The title compound (298 mg, 82%) was obtained as a white solid after recrystalization from MeOH. H NMR (400 MHz, DMSO) 2.94 (m, 4H), 3.65 (m, 4H), 7.96 (t, 1H), 8.03 (d, 1H), 8.13 (dd, 1H), 8.27-8.31 (m, 2H), 8.42 (d, 1H), 8.82 (d, 1H), 12.55 (s, 1H)

### 5-Cyano-2-({[3-(morpholin-4-ylsulfonyl)phenyl]carbonothioyl}amino)benzoic acid

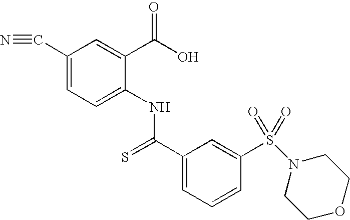

Prepared according to general method A and B: Methyl 5-cyano-2-{[3-(morpholin-4-ylsulfonyl)benzoyl]amino}benzoate (1.02 g, 2.38 mmol) and Lawesson's reagent (4.78 g, 11.8 mmol) afforded 532 g (50%) of the ester, 35527-bdw-118 as an orange solid. The ester (495 mg, 1.09 mmol) was hydrolyzed by general procedure B to afford 87 mg (20%) of an orange solid. ¹H NMR (300 MHz, DMSO-d₆) δ 9.72 (d, J=8 Hz, 1 H), 8.41 (d, J=2 Hz, 1 H), 8.27-8.25 (m, 2 H), 7.95 (dd, J=9, 6 Hz, 1 H), 7.90 (d, J=9 Hz, 1 H), 7.79 (t, J=6 Hz, 1 H).

### Example 7

### X—Y Derivatives

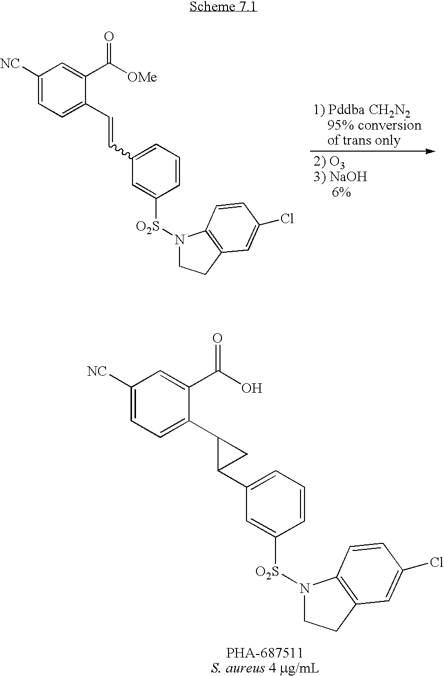

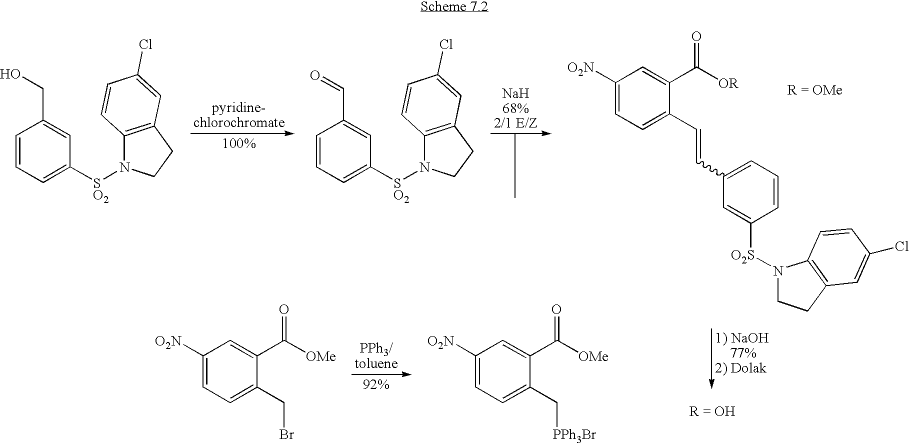

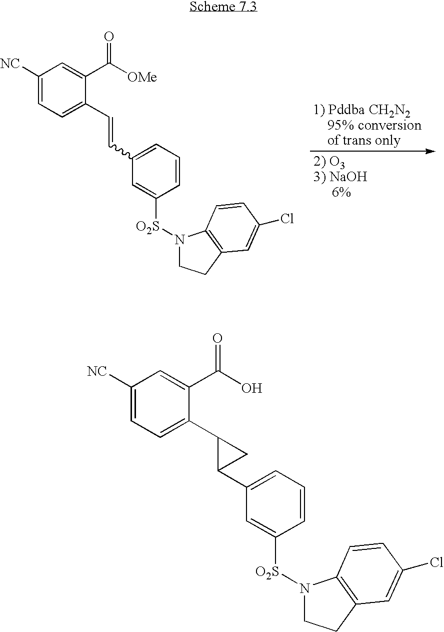

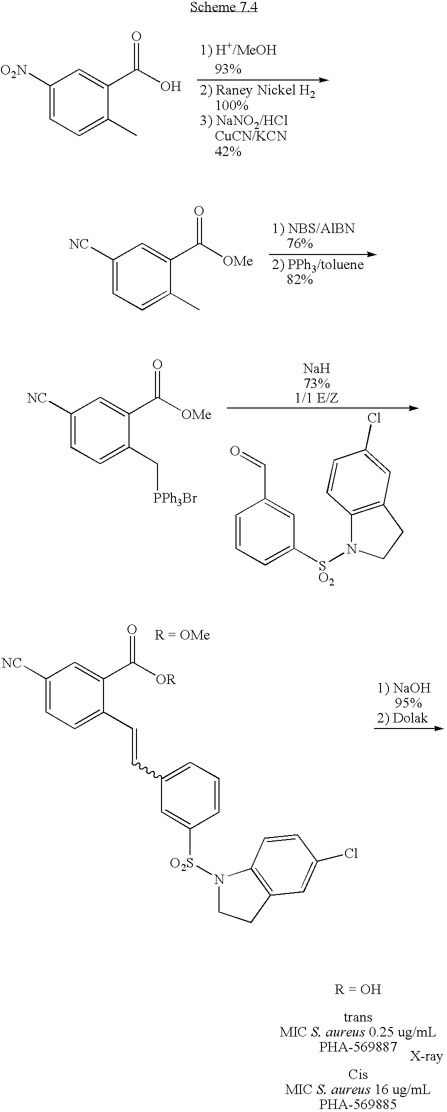

### Methyl 2-(bromomethyl)-5-cyanobenzoate

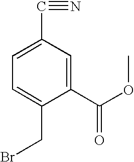

Methyl 5-cyanobenzoate (4.50 g, 25.6 mmol), NBS (5.03 g, 28.25 mmol) and AIBN (150 mg) were dissolved in dichloroethane (160 mL). The mixture was irratiated with a photolamp for 2 h. The mixture was cooled to rt and concentrated in vacuo. The residue was purified by silica gel chromatography (DCM/heptane 1/9, ¼, 1/1, 1/0) to afford 4.79 g (73%) of methyl 2-(bromomethyl)-5-cyanobenzoate. ¹H NMR (300 MHz, CDCl₃) δ 8.29 (d, J=1.7 Hz, 1 H), 7.79 (dd, J=8.0, 1.7 Hz, 1 H), 7.63 (d, J=8.0 Hz, 1 H), 4.97 (s, 2 H), 4.00 (s, 3 H).

### Methyl 2-{[bromo(triphenyl)phosphoranyl]methyl}-5-cyanobenzoate

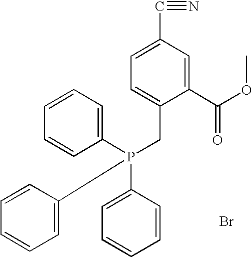

Methyl 2-(bromomethyl)-5-cyanobenzoate (2.80 g, 10.9 mol) was added to a solution of triphenylphosphine (2.87 g, 10.9 mmol) in toluene (50 mL). The resulting mixture was heated at reflux for 3 h, cooled to rt, the precipiate was isolated by filtration, washed with pentane to afford 4.64 g (82%) of methyl 2-{[bromo(triphenyl)phosphoranyl]methyl}-5-cyanobenzoate as a white solid. ¹H NMR (300 MHz, DMSO-d₆) δ 8.22 (s, 1 H), 8.08 (d, J=7.9 Hz, 1 H), 8.79-7.51 (m, 16 H), 5.63 (d, J=16.2 Hz, 2 H), 3.48 (s, 3 H).

### Methyl 2-methyl-5-nitrobenzoate

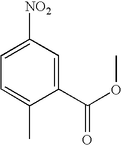

2-Methyl-5-nitrobenzoate (5.0 g, 27.6 mmol) was dissolved in MeOH (0.4 L) followed by the addition of H₂SO₄ (7 mL). The mixture was heated at reflux for 36 h, then cooled to rt and concentrated to ca 100 mL. The solution was diluted with MTBE neutralized with 6N NaOH, washed with 1N NaOH, brine, dried (MgSO₄), filtered and concentrated in vacuo to afford 4.72 g (87%) of methyl 2-methyl-5-nitrobenzoate as a white solid.

### Methyl 5-amino-2-methylbenzoate

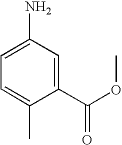

Methyl 2-methyl-5-nitrobenzoate (5.0 g, 25.6 mmol) was dissolved in EtOH with Raney nickel under a 35 psi atmosphere of H₂. The reaction was stirred for 20 h, then filtered through Celite washed with MeOH and concentrated in vacuo to afford 4.2 g (100%) of methyl 5-amino-2-methylbenzoate.

### Methyl 2-(bromomethyl)-5-nitrobenzoate

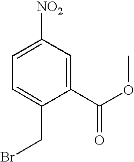

Methyl 2-methyl-5-nitrobenzoate (2.0 g, 10.2 mmol) NBS (2.73 g, 15.3 mmol) and AIBN (50 mg) were dissolved in dichloroethane (100 mL). The mixture was irratiated with a photolamp for 3 h. The mixture was cooled to rt and concentrated in vacuo. The residue was purified by silica gel chromatography (heptane/EtOAc 1/0, 19/1, 9/1) to afford 2.40 g (85%) of methyl 2-(bromomethyl)-5-nitrobenzoate.

### Methyl 2-{[bromo(triphenyl)phosphoranyl]methyl}-5-nitrobenzoate

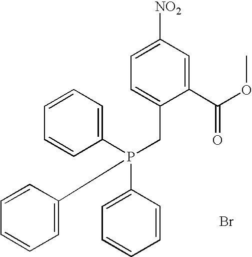

Methyl 2-(bromomethyl)-5-nitrobenzoate (666 mg, 2.43 mmol) was added to a solution of triphenylphosphine (640 mg, 2.4 mmol) in toluene (20 mL). The resulting mixture was heated at reflux for 3 h, cooled to rt, the precipitate was isolated by filtration, washed with pentane to afford 1.2 g (92%) of methyl 2-{[bromo(triphenyl)phosphoranyl]methyl}-5-nitrobenzoate as a white solid.

### Methyl 5-cyano-2-methylbenzoate

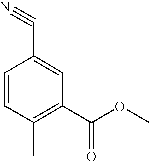

Methyl 5-amino-2-methylbenzoate (4.2 g, 25.4 mmol) was dissolved in MeOH/H₂O (20 mL/46 mL) was cooled with icebath followed by the addition of HCl (54 mL), NaNO₂ (2.63 g, 38.1 mmol, in H₂O 60 mL). The mixture was stirred for ½ h, then neutralized with solid NaHCO₃, extensive gasevolution. Then a cold mixture of KCN (2.48 g, 38 mmol) and CuCN (2.9 g, 33 mmol) in a H₂O (40 ml)/EtOAc (80 mL) was added. The reaction was stirred for ½ h, then filtered through Celite, extracted with EtOAc then washed with H₂O, brine, dried (MgSO₄), filtered anc concentrated in vacuo. The residue was purified by silica gel chromatography (heptane/DCM 19/1, 9/1, 1/1, 1/0) to afford 1.89 g (42%) of a white solid. ¹H NMR (300 MHz, CDCl₃) δ 8.23 (d, J=1.7 Hz, 1 H), 7.68 (dd, J=1.8, 7.9 Hz, 1 H), 7.38 (d, J=7.9 Hz, 1 H), 3.94 (s, 3 H).

### Methyl 5-chloro-2-methylbenzoate

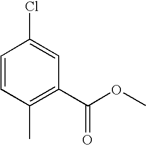

Methyl 5-chloro-2-methylbenzoate (25 g, 147 mmol) was dissolved in MeOH (0.6 L) followed by the addition of H₂SO₄ (50 mL). The mixture was heated at reflux for 12 h, then cooled to rt and concentrated to ca 200 mL. The solution was diluted with MTBE, washed with H2O, 1N NaOH, brine, dried (MgSO₄), filtered and concentrated in vacuo to afford 24.9 g (92%) of methyl 5-chloro-2-methyl-benzoate as a white solid. ¹H NMR (300 MHz, CDCl₃) δ 7.91 (d, J=2.3 Hz, 1 H), 7.38 (dd, J=2.3, 8.1 Hz, 1 H), 7.19 (d, J=8.2 Hz, 1 H), 3.91 (s, 3 H).

### Methyl 2-(bromomethyl)-5-chlorobenzoate

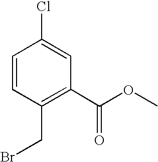

Methyl 5-chloro-2-methyl benzoate (10.0 g, 54 mmol) NBS (10.6 g, 59.5 mmol) and AIBN (200 mg) were dissolved in dichloroethane (300 mL). The mixture was irratiated with a photolamp for 2 h. The mixture was cooled to rt and concentrated in vacuo. The residue was purified by silica gel chromatography (heptane/DCM 9/1, 4/1, 1/1) to afford 11.8 g (83%) of methyl 2-(bromomethyl)-5-chlorobenzoate. ¹H NMR (300 MHz, CDCl₃) δ 7.98 (d, J=2.1 Hz, 1 H), 7.49 (dd, J=2.2, 8.2 Hz, 1 H), 7.43 (d, J=8.2 Hz, 1 H), 4.93 (s, 2 H), 3.97 (s, 3 H).

### Methyl 2-{[bromo(triphenyl)phosphoranyl]methyl}-5-chlorobenzoate

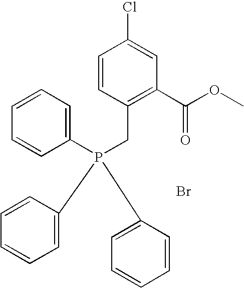

Methyl 2-(bromomethyl)-5-chlorobenzoate (11.8 g, 44.6 mmol) was added to a solution of triphenylphosphine (11.6 g, 44.6 mmol) in toluene (400 mL). The resulting mixture was heated at reflux for 3 h, cooled to rt, the precipitate was isolated by filtration, washed with pentane to afford 18.7 g (80%) of methyl 2-{[bromo(triphenyl)phosphoranyl]methyl}-5-clorobenzoate as a white solid. ¹H NMR (300 MHz, CDCl₃) δ 7.85-7.68 (m, 5 H), 7.63-7.57 (m, 12 H), 7.38-7.28 (m, 1 H), 5.88 (d, J=15.0 Hz, 2 H), 3.43 (s, 3 H).

### 2-((Z)-2-{3-[(5-Chloro-2,3-dihydro-1H-indol-1-yl)sulfonyl]phenyl}ethenyl)-4-nitrobenzoic acid

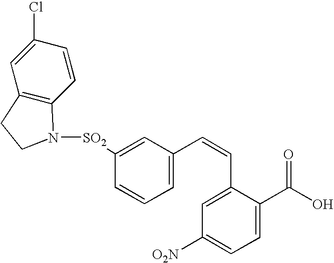

Methyl 2-{[bromo(triphenyl)phosphoranyl]methyl}-5-nitrobenzoate (1.20 g, 2.24 mmol) was added to DMSO (30 mL) followed by NaH (100 mg, 2.4 mmol), gas evolution was observed, and the resulting mixture was heated at 60° C. for 2 h. Then 3-[(5-chloro-2,3-dihydro-1H-indol-1-yl)sulfonyl]benzaldehyde (800 mg, 2.5 mmol) in toluene (50 mL) was added the reaction was stired at rt for 2 h, then at 60° C. for 2 h. The mixture was diluted with MTBE, washed with H₂O, brine, dried (MgSO₄), filtered and concentrated in vacuo. The residue was purified by silica gel chromatography (DCM/MeOH 1/0, 19/1) to afford 760 mg (68%) of a Z/E mixture (4/1). The solid was dissolved in THF/MeOH (2/1, 60 mL) and 6N NaOH (6 mL) was added. The mixture was stirred at rt for 1 h, then diluted with MTBE, washed with 1N HCl, H₂O, brine, dried (MgSO₄), filtered and concentrated in vacuo. The residue was purified by silica gel chromatography (DCM/MeOH 1/0, 19/1) to afford 574 mg (77%). This was recrystallized from MeOH. The mother liquid was recrystallized several time to afford 182 mg. ¹H NMR (400 MHz, DMSO-d₆) δ 8.66 (d, J=2.4 Hz, 1 H), 8.14-8.12 (m, 1 H), 7.63-7.57 (m, 1 H), 7.49-7.18 (m, 8 H), 6.84 (d, J=12.3 Hz, 1 H), 3.65 (t, J=8.4 Hz, 2 H), 2.86 (t, J=8.4 Hz, 2 H).

### 2-((E)-2-{3-[(5-Chloro-2,3-dihydro-1H-indol-1-yl)sulfonyl]phenyl}ethenyl)-4-nitrobenzoic acid

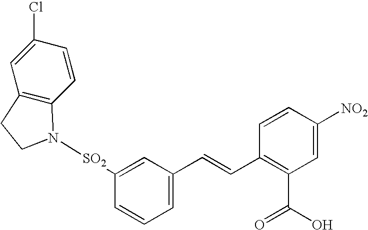

¹H NMR (400 MHz, DMSO-d₆) δ 8.56 (d, J=2.4 Hz, 1 H), 8.33-8.31 (m, 1 H), 8.18 (d, J=16.5 Hz, 1 H), 8.08-8.03 (m, 2 H), 7.92 (d, J=7.8 Hz, 1 H), 7.75-7.73 (m, 1 H), 7.62 (t, J=7.8 Hz, 1 H), 7.51-7.47 (m, 2 H), 7.27-7.23 (m, 2 H), 3.98 (t, J=8.4 Hz, 2 H), 2.94 (t, J=8.4 Hz, 2 H).

### 5-Chloro-2-((E)-2-{3-[(5-chloro-2,3-dihydro-1H-indol-1-yl)sulfonyl]phenyl}ethenyl)benzoic acid

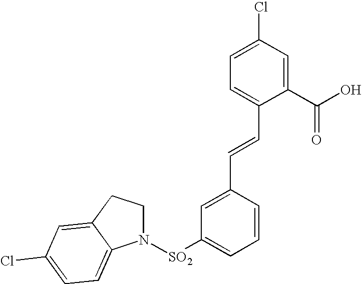

Methyl 2-{[bromo(triphenyl)phosphoranyl]methyl}-5-chlorobenzoate (392 mg, 0.74 mmol) was added to THF (10 mL) in icebath, followed by LiCl (260 mg, 6.2 mmol), and n-BuLi (300 μL, 0.74 mmol). The reaction was stirred at rt for 10 min, then 3-[(5-chloro-2,3-dihydro-1H-indol-1-yl)sulfonyl]benzaldehyde (200 mg, 0.6 mmol) was added and the reaction was stirred at rt for 2 h. The mixture was diluted with MTBE, washed with H₂O, brine, dried (MgSO₄), filtered and concentrated in vacuo. The residue was purified by silica gel plug (DCM) to afford 271 mg of a Z/E mixture. The solid was dissolved in toluene (10 mL) followed by the addition of thiophenol (32 μL, 0.28 mmol) and AIBN (14 mg, 0.08 mmol). The reaction was heated at reflux for 12 h, then concentrated in vacuo. The residue was dissolved in THF (60 mL) and 6N NaOH (5 mL) was added. The mixture was stirred at 100° C. for 4 h, then diluted with MTBE, washed with 1N HCl, H₂O, brine, dried (MgSO₄), filtered and concentrated in vacuo. The residue was recrystallized from MeOH to afford 123 mg. ¹H NMR (300 MHz, DMSO-d₆) δ 7.97-7.85 (m, 5 H), 7.70-7.60 (m, 3 H), 7.48 (d, J=8.2 Hz, 1 H), 7.33-7.24 (m, 3 H), 3.97 (t, J=8.4 Hz, 2 H), 2.93 (t, J=8.4 Hz, 2 H).

### 5-Cyano-2-((E)-2-{3-[(5-chloro-2,3-dihydro-1H-indol-1-yl)sulfonyl]phenyl}ethenyl)benzoic acid

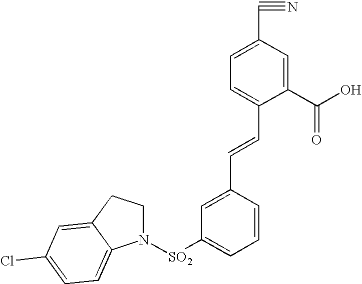

Methyl 2-{[bromo(triphenyl)phosphoranyl]methyl}-5-cyanobenzoate (1.36 g, 2.6 mmol) was added to DMSO (20 mL) followed by NaH (105 mg, 2.6 mmol), gas evolution was observed, and the resulting mixture was heated at 60° C. for 2 h. Then 3-[(5-chloro-2,3-dihydro-1H-indol-1-yl)sulfonyl]benzaldehyde (564 mg, 1.7 mmol) in toluene (50 mL) was added the reaction was stirred at rt for 1 h, then at 60° C. for 1 h. The mixture was diluted with MTBE, washed with H₂O, brine, dried (MgSO₄), filtered and concentrated in vacuo. The residue was purified by silica gel chromatography (DCM/heptane 1/1, 1/0) to afford 616 mg (73%) of a Z/E mixture. The solid was dissolved in THF (60 mL) and iN NaOH (10 mL) was added. The mixture was stirred at rt for 12 h, then diluted with MTBE, washed with 1N HCl, H₂O, brine, dried (MgSO₄), filtered and concentrated in vacuo to afford 567 mg (95%). This was purified by preparative reverse phase HPLC to afford 144 mg of pure (E) and 99 mg of (Z). ¹H NMR (300 MHz, DMSO-d₆) δ 8.24 (s, 1 H), 8.05-7.89 (m, 5 H), 7.76-7.73 (m, 1 H), 7.63 (t, J=7.7 Hz, 1 H), 7.49-7.44 (m, 2 H), 7.27-7.24 (m, 2 H), 3.98 (t, J=8.5 Hz, 2 H), 2.93 (t, J=8.5 Hz, 2 H).

### 5-Cyano-2-((Z)-2-{3-[(5-chloro-2,3-dihydro-1H-indol-1-yl)sulfonyl]phenyl}ethenyl)benzoic acid

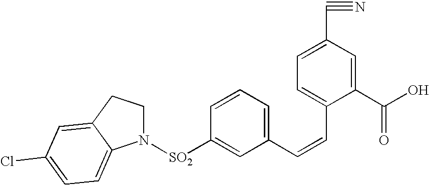

¹H NMR (300 MHz, DMSO-d₆) δ 8.33 (d, J=1.7 Hz, 1 H), 7.84-7.81 (m, 1 H), 7.59-7.57 (m, 1 H), 7.47-7.12 (m, 8 H), 6.82 (d, J=12.2 Hz, 1 H), 3.66 (t, J=8.5 Hz, 2 H), 2.88 (t, J=8.3 Hz, 2 H).

### 2-(2-{3-[(5-Chloro-2,3-dihydro-1H-indol-1-yl)sulfonyl]phenyl}cyclopropyl)-5-cyanobenzoic acid

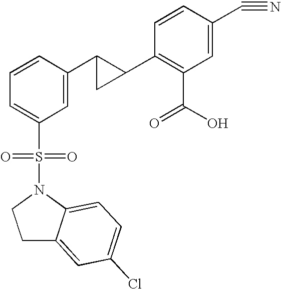

Diazomethane solution (400 ml, from 36 g Dizald, for procedure see Denmark, S. E.; Stavenger, R. A.; Faucher, A-M.; Edwards, J. P. J. Org. Chem. 1997, 62, 3375) was added to a solution of methyl 5-cyano-2-(2-{3-[(5-chloro-2,3-dihydro-1H-indol-1-yl)sulfonyl]phenyl}ethenyl)benzoate (850 mg, 1.7 mmol) and Pdba (100 mg) in DCM (150 mL). Extensive gas evolution was observed, the resulting mixture was stirred for 12 h, then HOAc (5 mL) was added, filtered through Celite, washed with 1 n NaOH, brine, dried (MgSO₄), filtered and concentrated in vacuo to afford 982 mg of a solid. The residue in DCM (100 mL) was cooled with icebath and O3 was bubbled through for 30 min. Then NaBH4 (500 mg) was added and the mixture was stirred for 30 min at rt. The mixture was passed through a silica plug and concentrated in vacuo. The residue was purified by silica gel chromatography (heptane/DCM 9/1, 4/1, 1/1, 0/1) to afford 124 mg of the desired cyclopropane. The solid was dissolved in THF (25 mL) and 6N NaOH (5 mL) was added, the resulting mixture was stirred for 16 h at rt, then diluted with MTBE, washed with 1N HCl, H₂O, brine, dried (MgSO₄) filtered and concentrated in vacuo. The residue was purifed by silica gel chromatography (DCM/MeOH 1/0, 19/1, 9/1, 4/1) to afford 51 mg (6%). ¹H NMR (300 MHz, CDCl₃) δ 8.27 (d, J=1.6 Hz, 1 H), 7.79 (d, J=8.1 Hz, 1 H), 7.60-7.57 (m, 3 H), 7.40 (d, J=5.3 Hz, 2 H), 7.25 (d, J=8.1 Hz, 1 H), 7.18-7.16 (m, 1 H), 7.04 (s, 1 H), 3.95 (t, J=8.3 Hz, 2 H), 3.18-3.13 (m, 1 H), 2.87 (t, J=8.3 Hz, 2 H), 2.24-2.19 (m, 1 H), 1.65-1.60 (m, 1 H), 1.54-1.49 (m, 1 H).

### Example 8

In other embodiments, the invention includes benzoxazine derivatives of the formula 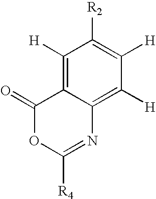
wherein
R₂ is an electron withdrawing group; and
R₄ is an optionally substituted aryl, provided that the aryl is not simultaneously substituted with a sulfonamide and a urea or thiourea, and fuirther provided that the aryl is not solely substituted at the ortho-position relative to Y.

### 2-{3-[(5-Chloro-2,3-dihydro-1H-indol-1-yl)sulfonyl]phenyl}-4-oxo-4H-3,1-benzoxazine-6-carbonitrile

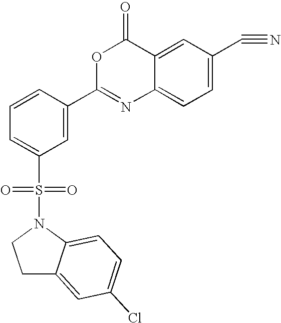

2-({3-[(5-Chloro-2,3-dihydro-1H-indol-1-yl)sulfonyl]benzoyl}amino)-5-cyanobenzoic acid (PHA-524523, 884 m, 1.84 mmol) was dissolved in anhydrous THF (30 mL) and Et₃N (0.563 mL, 4.04 mmol) under N₂. Addition of ethyl chloroformate (0.193 mL, 2.02 mmol, Aldrich) to the yellow solution produced a white precipitate, which was stirred overnight at RT. The solvent was evaporated and the resultant residue suspended in CH₂Cl₂ (100 mL). The organic layer was washed 2× with 1.0M HCl, 1× with water and 1× with brine (100 mL each). The crude product was purified on a Biotage Flash 40M (90 g) silica cartridge using a step gradient of 0-1% CH₃OH in CH₂Cl₂. After evaporation the resultant solid was dried under vacuum at 100° C. to afford 280 mg (33%) of white solid. ¹H NMR (400 MHz, DMSO-d₆) δ 8.65 (d, J=1.9 Hz, 1 H), 8.52 (s, 1 H), 8.47 (d, J=8.0 Hz, 1 H), 8.36 (dd, J=8.4, 1.9 Hz, 1 H), 8.11 (d, J=8.4 Hz, 1 H), 7.92 (d, J=8.4 Hz, 1 H), 7.85 (t, J=7.9 Hz, 1 H), 7.53 (d, J=8.6 Hz, 1 H), 7.30 (d, J=8.6 Hz, 1 H), 7.26 (s, 1 H), 3.99 (t, J=8.4 Hz, 2 H), 2.94 (t, J=8.4 Hz, 2 H).

### Example 9

### Library Synthesis

General Experimental

¹H NMR spectra were measured using a Bruker AVANCE 300 spectrometer at rt in DMSO-d₆ at an operating frequency of 300.13 MHz and are referenced to residual DMSO-d₆ (2.54 ppm) unless otherwise noted. All coupling constants are reported in Hz. All non-combinatorial reactions were performed under a nitrogen atmosphere.
Synthetic Procedures Using Wang Resins

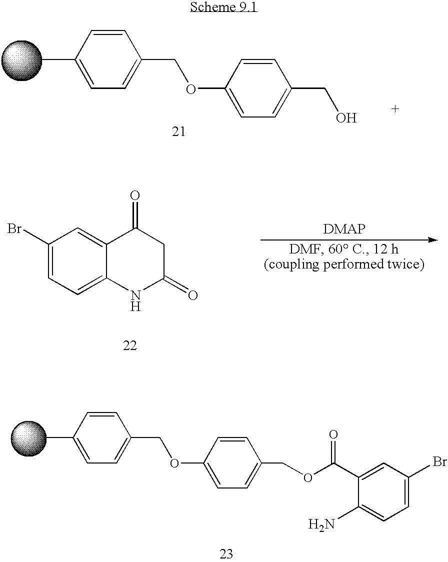

To a dry, 2-L polypropylene bottle equipped with a nitrogen inlet and an overhead stirrer was added Wang resin (21, 38.6 g, 49.7 mmol, 1.3 mmol/g, Novabiochem), DMF (600 mL), 5-bromoisatoic anhydride (22, 60.0 g, 248 mmol, dissolved in 100 mL of DMF), and DMAP (30.3 g, 248 mmol, dissolved in 100 mL of DMF). The reaction was heated under nitrogen to 65° C. and stirred for 12 h. The reaction was then filtered and washed as follows: DMF, CH₃CN, DMF, CH₃CN, DMF, CH₃CN, CH₃CN, CH₂Cl₂, CH₃CN, CH₂Cl₂, CH₃CN, and CH₂Cl₂. The washed resin was transferred back to the 2-L reaction flask and treated a second time with DMF (600 mL), 5-bromoisatoic anhydride (60.0 g, 248 mmol, dissolved in 100 mL of DMF), and DMAP (30.3 g, 248 mmol, dissolved in 100 mL of DMF). The reaction was stirred at 65° C. for 4 h and then filtered and washed with DMF, CH₃CN, DMF, CH₃CN, DMF, CH₃CN, CH₃CN, CH₂Cl₂, CH₃CN, CH₂Cl₂, CH₃CN, and CH₂Cl₂) to afford (48.57 g) of 23 as an off-white resin. CNH analysis: Calcd (1.3 mmol): N, 1.82, Found: N, 1.67% (loading=1.2 mmol/g).

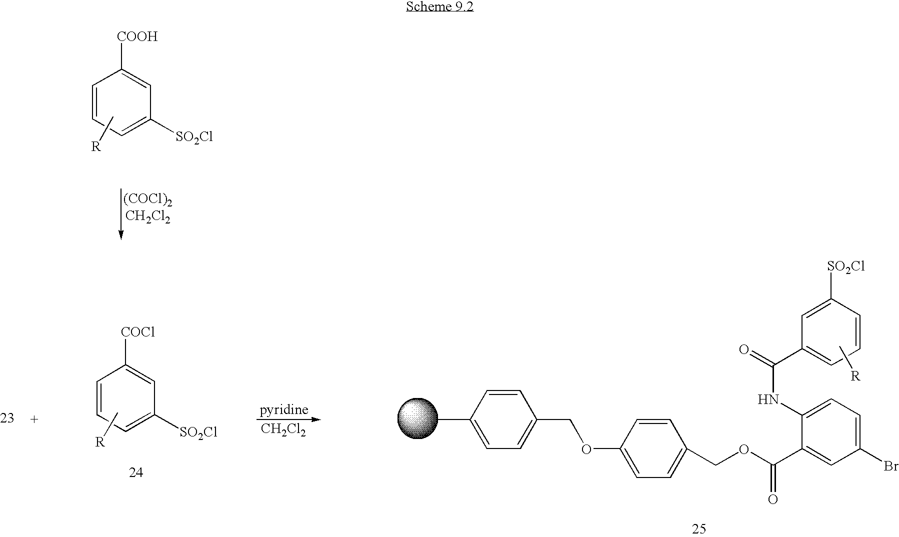

To a suspension of a 3-chlorosulfonylbenzoic derivative (24, 31.6 mmol) in CH₂Cl₂ (100 mL) was added DMF (two drops), followed by oxalyl chloride (31.6 mL of a 2 M solution in CH₂Cl₂, 63.2 mmol) under a nitrogen atmosphere. Gas evolution and disappearance of the suspension was noted during the course of the reaction. After the reaction was stirred for 18 h, the acid chloride was concentrated to dryness, azeotroped with toluene (2×25 mL), and then placed on a high vacuum. Dry anthranilic acid-derivatized Wang resin (7.0 g, 8.4 mmol) was added to an 8-oz wide-mouth bottle, followed by CH₂Cl₂ (35 mL) and pyridine (35 mL). The acid chloride was dissolved in CH₂Cl₂ (20 mL) and added to resin, effecting HCl (g) evolution. The reaction jar was flushed with nitrogen, capped and shaken for 4 h. The resin was then filtered and washed (CH₂Cl₂, MeCN, CH₂Cl₂, MeCN, CH₂Cl₂, MeCN, CH₂Cl₂, CHCl₃, CH₂Cl₂, THF, MeCN, THF, CH₂Cl₂; 50 mL each wash) to afford 25 as a tan resin.

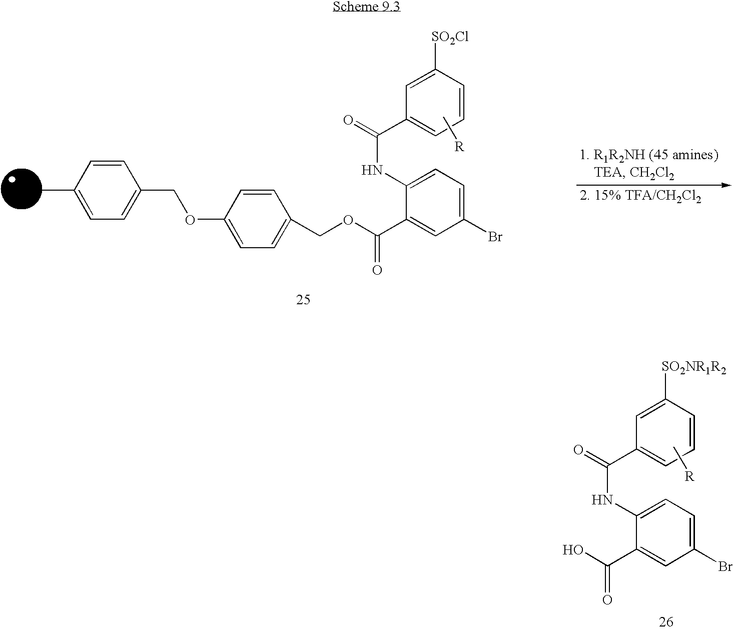

Sulfonyl chloride resin (25, 50 mg, 60 μmol) was added down the columns of a 96-well microtiter filter plate using a CH₂Cl₂ isopycnic slurry. After draining the wells, the plate was inserted into a solid phase reaction apparatus. Amines (300 μL of a 0.75 M solution, 225 μmol) were then added across the rows, followed by triethylamine (250 μL of a 1.8 M solution) and CH₂Cl₂ (250 μL). The plate was capped and spun on an overhead rotisserie for 16 h. After removal of the plate from the solid phase reaction apparatus, the wells drained and each well was washed (DMF, CH₃CN, DMF, CH₃CN, DMF, CH₃CN, CH₃CN, CH₂Cl₂, CH₃CN, CH₂Cl₂, CH₃CN, and CH₂Cl₂).

The plate was again inserted into the solid phase reaction apparatus and a 15% solution of TFA in CH₂Cl₂ (625 μL) was added. The plate was spun on an overhead rotisserie for 3 h and the crude sulfonamides were then drained into a 1-mL 96-well plate. The resin was washed with CH₂Cl₂ (1.5 mL) and the washes collected in additional 1-mL plates. LC/MS samples were prepared by transferring 40 μL of solution to a separate 96-well plate, concentrating the samples and then dissolving in DMSO (125 μL) and diluting with acetonitrile (750 μL).

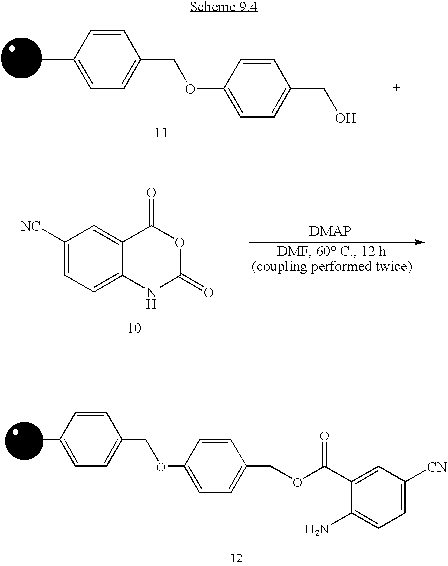

To a dry, 2-L polypropylene bottle equipped with a nitrogen inlet and an overhead stirrer was added Wang resin (11, 15.1 g, 21.1 mmol, 1.4 mmol/g, Novabiochem), DMF (500 mL), 5-cyanoisatoic anhydride (10, 20.0 g, 106 mmol, dissolved in 100 mL DMF), and DMAP (13.0 g, 106 mmol, dissolved in 100 mL DMF). The mixture was heated under nitrogen to 53° C. and stirred for 16 h. The reaction was then filtered and washed with 500 μL of the following solvents: DMF, CH₃CN, DMF, CH₃CN, DMF, CH₃CN, DMF, DMF, CH₂Cl₂, CH₂Cl₂, CH₂Cl₂, CH₂Cl₂, DMF, DMF, and DMF. The resin was transferred back to the 2-L reaction flask and treated a second time with DMF (500 mL), 5-cyanoisatoic anhydride (10, 20.0 g, 106 mmol, dissolved in 100 mL DMF), and DMAP (13.0 g, 106 mmol, dissolved in 100 mL DMF). The reaction was stirred at 60° C. for 22 h and then filtered and washed with 500 μL of CH₃CN, DMF, CH₃CN, DMF, CH₃CN, DMF, CH₃CN, CH₂Cl₂, CH₃CN, CH₂Cl₂, CH₃CN, CH₂Cl₂, to afford 15.3 g of 12 as a pale yellow resin. Elemental analysis: N, 3.20% (loading=1.14 mmol/g).⁵

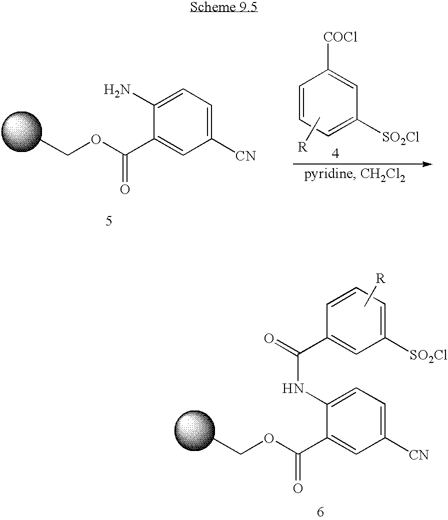

Dry 5-cyano anthranilic acid-derivatized Wang resin (5, 5.0 g, 1.0 mmol/g loading, 5.0 mmol) was added to an 8-oz wide mouth bottle, followed by CH₂Cl₂ (30 mL) and pyridine (30 mL). The acid chloride (4) was dissolved in CH₂Cl₂ (30 mL) and added to the resin, effecting HCl (gas) evolution. The jar was flushed with nitrogen, capped, and shaken for 64 h. The resin was then filtered and washed (DMF, CH₃CN, DMF, CH₃CN, DMF, CH₃CN, DMF, THF, THF, THF, CH₃CN, CH₂Cl₂, CH₃CN, CH₂Cl₂, CH₃CN, CH₂Cl₂; 400 mL each wash) to afford 6.

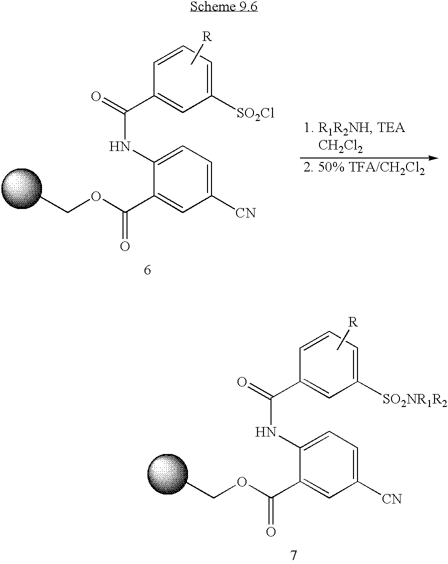

Sulfonyl chloride resin (6, 50 mg, 50 μmol) was added to the wells of a 96-well filter plate using a CH₂Cl₂ isopycnic slurry. After draining the wells, the plate was inserted into a solid phase reaction apparatus. Amines (250 μL of a 2 M solution, 500 μmol) were then added, followed by triethylamine (250 μL of a 2 M solution) and CH₂Cl₂ (250 μL). The plate was then capped and spun on an overhead rotisserie for 20 h. After removal of the plate from the solid phase reaction block, the wells were drained and washed (DMF, CH₃CN, DMF, CH₃CN, DMF, CH₃CN, H₂O, THF, H₂O, THF, H₂O, THF, CH₃CN, CH₂Cl₂, CH₃CN, CH₂CI₂, CH₃CN, CH₂Cl₂; 375 μL each wash).

The plate was again inserted into the solid phase reaction apparatus and a 50% solution of TFA in CH₂Cl₂ (500 μL) was added. The plate was spun on an overhead rotisserie for 3 h and the crude sulfonamides (7) were then drained into a standard 96-well plate. The resin was washed with 250 μL of additional 50% TFA solution. Products were concentrated under nitrogen and then analyzed by LC/MS (see general LC/MS procedure). The crude samples were dissolved in THF, and eluted through a plug of Celite®. LC/MS showed a reduced amount of impurity in all of the samples. The samples that were less than 70% pure were then eluted through a plug of silica gel using THF as the mobile phase and the samples were analyzed by LC/MS.

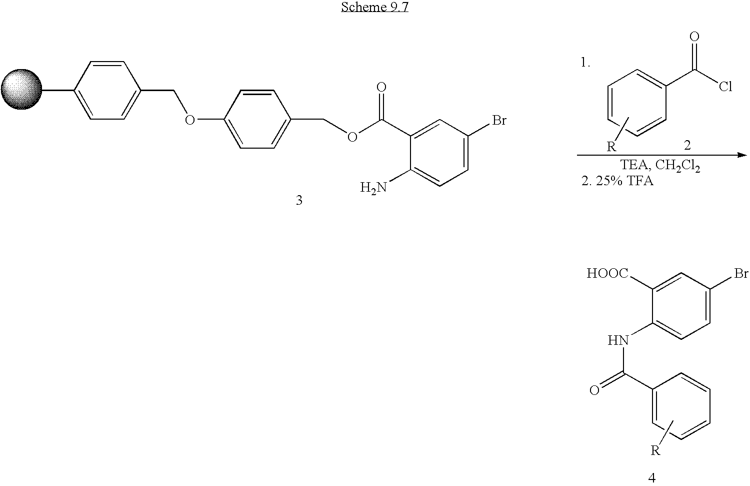

To a standard 96-well filter plate was added 50 mg (60 μmol) of 5-bromoanthanilic acid derivatized Wang resin as an isopycnic solution in CH₂Cl₂ (3). After the wells were drained, the plate was inserted into a plate clamp assembly. The acid chloride diversity set (2) was dissolved in CH₂Cl₂ (300 μL) and added to the plate, followed by TEA (250 μL, 1 M CH₂Cl₂, 250 μmol) and CH₂Cl₂ (300 μL). The plate was capped and spun on an overhead rotisserie for 16 h. After removal of the plate from the plate clamp assembly, the wells were drained and the resin washed with 500 μL of the following solvents: CH₂Cl₂, MeCN, CH₂Cl₂, MeCN, CH₂Cl₂, MeCN, CH₂Cl₂, CHCl₃, CH₂Cl₂, THF, MeCN, THF, CH₂Cl₂. The plate was reinserted into the plate clamp assembly and the washed resin was treated with 750 μL of 25% TFA/CH₂Cl₂ solution for 3 h. The solution was then filtered from the Wang resin and collected in a separate plate to afford the crude amides (4). The plates were concentrated and analyzed by LC/MS (see general LC/MS procedure).

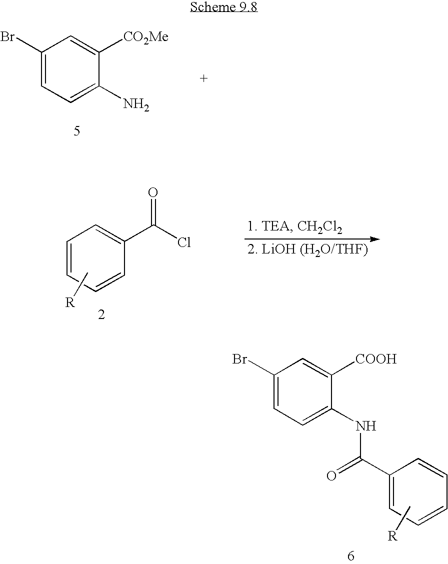

After concentration of the acid chloride solutions (2), methyl-2-amino-5-bromobenzoate 5, 125 μL, 1 M THF, 125 μmol/well) was added to the plate followed by potassium carbonate (1 mL, 0.38 M THF, 380 μmol/well). The reactions were capped, heated to 50° C. and shaken for 12 h. Triethylenetetramine resin (160 mg, 464 μmol) was added to the wells to scavenge the excess acid chloride and the plate spun for 2.5 h. The crude methyl esters were purified (if necessary) using a column consisting of basic alumina (ca 200 mg), SAX (ca 200 mg), and SCX (ca 400 mg, activated with 1% HOAc/MeOH) in descending order. The products were eluted with THF and the fractions analyzed by LC/MS.

LiOH [375 μL, 1 M H₂O/THF (50:50), 3 equiv)] was added to the esters and the plate was capped and spun for 1 h. The THF was then removed in vacuo. The crude solids were suspended in methyl ethyl ketone (MEK, 500 μL) and extracted with 2 N HCl (250 μL). The MEK layer was removed and the aqueous layer extracted again with MEK (500 μL). The combined organic layers were washed with 50% brine solution, passed through a plug of sodium sulfate, collected in a 1-mL plate, and dried under nitrogen to afford the amide products (6). The solids were then analyzed using LC/MS (see general LC/MS procedure).

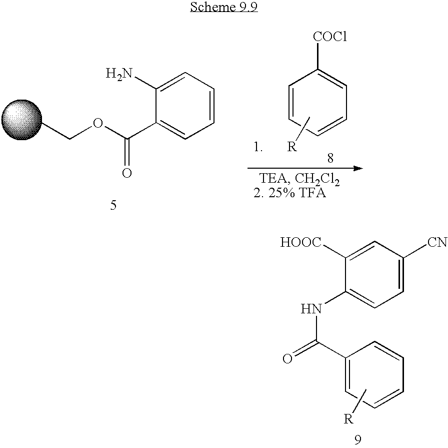

To each vial of an array of 1-mL vials arranged in a 96-well format was added 44 mg (50 μmol) of 5-cyanoanthranilic acid-derivatized Wang resin (5) as an isopycnic solution in CH₂Cl₂. The acid chloride diversity set² (8, 500 μmol) was dissolved in CH₂Cl₂ (300 μL) and added to the vials, followed by TEA (250 μL, 2 M CH₂Cl₂, 500 μmol), and CH₂Cl₂ (300 μL). The vials were capped, heated to 60° C., and shaken for 21 h. After completion of the reaction, the resin was transferred to a 96-well filter plate and washed with of the following solvents: DMF, CH₃CN, DMF, CH₃CN, DMF, CH₃CN, H₂O, THF, H₂O, THF, H₂O, THF, CH₃CN, CH₂Cl₂, CH₃CN, CH₂Cl₂. CH₃CN, CH₂Cl₂, CH₃CN, CH₂Cl₂ (500 μL/wash). The plate was placed into a clamp assembly and each well was treated with 500 μL of 50% TFA/CH₂Cl₂ solution for 2 h.³ The resultant solution was then filtered from the Wang resin, collected in a separate plate, and dried under nitrogen to afford the crude amides (9).

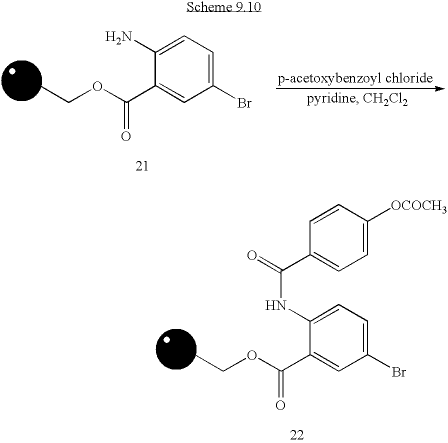

Resin-bound 4-Acetoxybenzoyl Anthranilic Acid. To a 500-mL round bottom flask under nitrogen was added 4-acetoxybenzoic acid (20.7 g, 115.5 mmol) and CH₂Cl₂ (200 mL). After cooling the flask to 0° C., oxalyl chloride (57.8 mL of a 2 M solution, 116 mmol) and a few drops of DMF were added. The reactions were allowed to warm to room temperature and stirred for 3 h. These solutions were directly transferred to a 2-L serum flask containing 5-bromoanthranillic acid resin (21, 7.0 g, 7.7 mmol), pyridine (100 mL) and CH₂Cl₂ (100 mL). The resulting mixtures were stirred under nitrogen overnight and then filtered into a glass fritted funnel. The resin was then washed with DMF (3×100 mL), CH₂Cl₂ (5×100 mL), and MeOH (5×100 mL). The resin was then dried in a vacuum oven at 60° C. for 72 h to afford 22 (8.0 g). A sample was cleaved from the resin by stirring in 25% TFA in CH₂Cl₂ for 3 h: ¹H NMR (acetone-d₆) δ 2.31 (s, 3H), 7.35 (d, J=2.1, 1H), 7.37 (d, J=2.0, 1H), 7.82 (d, J=2.5, 1H), 7.86 (d, J=2.5, 1H), 8.07 (dd, J=2.1, 8.7, 1H), 8.27 (d, J=2.5, 1H), 8.90 (d, J=9.0, 1H).

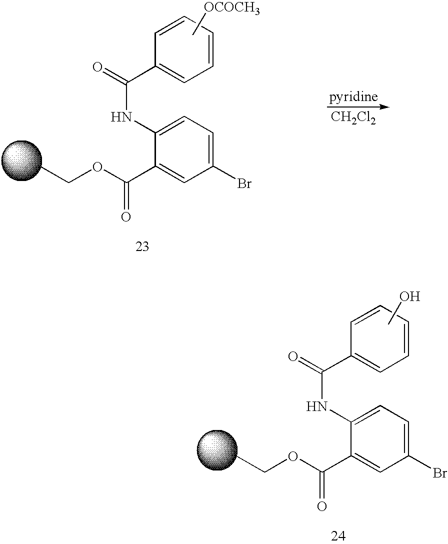

Resin-bound 4-Hydroxybenzoyl Anthranilic Acid. To a 250-mL serum bottle was added acetoxy resin 23 (7.0 g, 7.7 mmol), CH₂Cl₂ (70 mL), and piperidine (150 mL, 2 M CH₂Cl₂). The slurry was stirred for 2 h at room temperature. The resins were then filtered and washed with DMF (3×100 mL), Et₃N (1 M CH₂Cl₂, 2×100 mL), and MeOH (2×100 mL), CH₂Cl₂ (40 mL), MeOH (40 mL), CH₂Cl₂ (40 mL), MeOH (40 mL), CH₂Cl₂ (40 mL), and MeOH (40 mL). The resin was then dried for 72 h in a vacuum oven at room temperature to afford 6.6 g of 24 as a yellow resin.

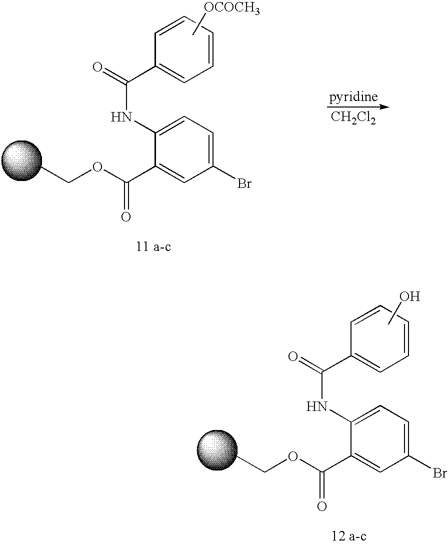

Synthesis of Resin-Bound Phenol 12a. To a 200-mL Wheaton bottle equipped with an overhead stirrer was added resin-bound acetate (11a, 5.0 g) followed by piperidine (150 mL of a 2 M solution in CH₂Cl₂, 300 mmol). The reaction was stirred for 2 h at room temperature. The resin was then filtered from the reaction mixture, washed with DMF, DMF, DMF, Et₃N (1 M in CH₂Cl₂), MeOH, CH₂Cl₂, MeOH, CH₂Cl₂, MeOH, CH₂Cl₂, MeOH, CH₂Cl₂, MeOH, CH₂Cl₂, (50 mL each), and dried in a vacuum oven at 50° C. overnight to afford resin-bound phenol 12a as a brownish solid.

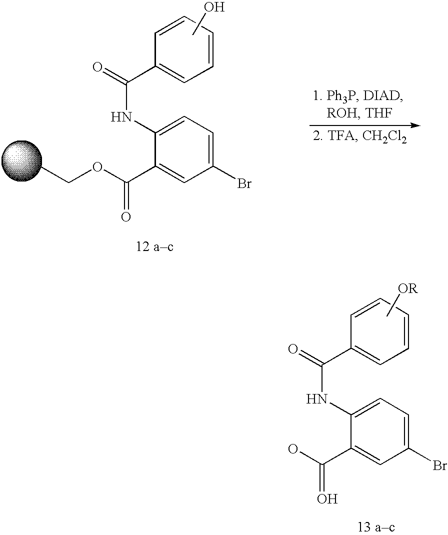

Mitsunobu Reaction (Procedure A). To each well of a fritted 96-well plate was added phenol resin (12a-c, 20.0 mg, 20.0 μmol) as an isopycnic solution (20% THF in CH₂Cl₂) and the plate was placed in a solid phase reaction assembly. The alcohol diversity element (200 μL of a 1 M solution in THF, 200 μmol) was then added, followed by triphenylphosphine (200 μL of a 1 M solution in THF, 200 μmol). The wells were flushed with nitrogen, capped, and placed in the −20° C. freezer for 1 h. While in the freezer, DIAD [200 μL of a cooled (−20° C.), freshly made 1 M solution in THF] was added to each well. The plate was removed from the freezer after 1 h and then spun on the rotisserie for 16 h. The reaction mixture was drained from the plate and the resin then washed with THF, THF, THF (the plate was capped and spun on an overhead rotisserie for 30 min), THF, MeOH, THF, MeOH, THF, MeOH, MeOH, CH₂Cl₂, MeOH, CH₂Cl₂, MeOH, CH₂Cl₂, MeOH, CH₂Cl₂, MeOH, CH₂Cl₂, MeOH, CH₂Cl₂, MeOH (the plate was capped and spun on an overhead rotisserie for 30 min), CH₂Cl₂, CH₂Cl₂, CH₂Cl₂; 500 μL each solvent. The crude aryl ethers were then cleaved from the resin using 500 μL of 50% TFA in CH₂Cl₂. The resulting products (13a-c) were concentrated under a nitrogen stream and analyzed by HPLC/MS.

Mitsunobu Reaction (Procedure B). To 72 wells of a fritted 96-well plate was added phenol resin as an isopycnic solution (12a-c, 20.0 mg, 20.0 μmol) and the plate was placed in a solid phase reaction assembly. The alcohol diversity element (200 μL of a 1 M solution in THF, 200 μmol) was then added, followed by triphenylphosphine (200 μL of a 1 M solution in THF, 200 μmol) and Et₃N (200 μL of a 1 M solution in THF, 200 μmol). The wells were flushed with N₂, capped, and placed in the −20° C. freezer for 1 h. While in the freezer, DIAD [200 μL of a cooled (−20° C.), freshly made 1 M solution in THF] was added to each well. The plate was removed from the freezer after an hour and then spun on the rotisserie for 16 h. The reaction mixture was drained from the plate and the resin then washed with THF, THF, THF (the plate was capped and spun on an overhead rotisserie for 30 min), THF, MeOH, THF, MeOH, THF, MeOH, MeOH, CH₂Cl₂, MeOH, CH₂Cl₂, MeOH, CH₂Cl₂, MeOH, CH₂Cl₂, MeOH, CH₂Cl₂, MeOH, CH₂Cl₂, MeOH (the plate was capped and spun on an overhead rotisserie for 30 min), CH₂Cl₂, CH₂Cl₂, CH₂Cl₂; 500 μL each solvent. The crude aryl ethers were then cleaved from the resin using 500 μL of 50% TFA in CH₂Cl₂. The resulting products (13a-c) were concentrated under a nitrogen stream and analyzed by HPLC/MS.

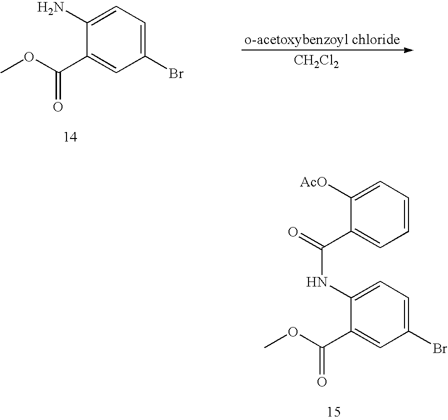

Synthesis of Acetate 15. To a 250-mL round bottom flask was added a solution of methyl-2-amino-5-bromobenzoate [14, 5.0 g, 21.7 mmol dissolved in pyridine (10 mL) and CH₂Cl₂ (10 mL)], followed by o-acetoxybenzoyl chloride⁵ (4.7 g, 33.8 mmol dissolved in 60 mL of CH₂Cl₂). The mixture was stirred overnight under a nitrogen atmosphere. Polyamine resin (4.0 g) was then added to the reaction mixture and the reaction was stirred for 4 h. After filtration and concentration of the reaction mixture, a white residue was obtained. The residue was recrystallized from CH₂Cl₂ to afford 8.0 g (94%) of 15 as a white solid: ¹H NMR (DMSO-d₆) δ 2.24 (s, 3H), 3.86 (s, 3H), 7.30 (d, J=8.1, 1H), 7.46 (dt, J=1.1, 7.6, 1H), 7.66 (dt, J=1.7, 8.0, 1H), 7.82 (dd, J=1.7, 7.7, 1H), 7.86 (dd, J=2.5, 8.9, 1H), 8.06 (d, J=2.5, 1H), 8.38 (d, J=8.9, 1H).

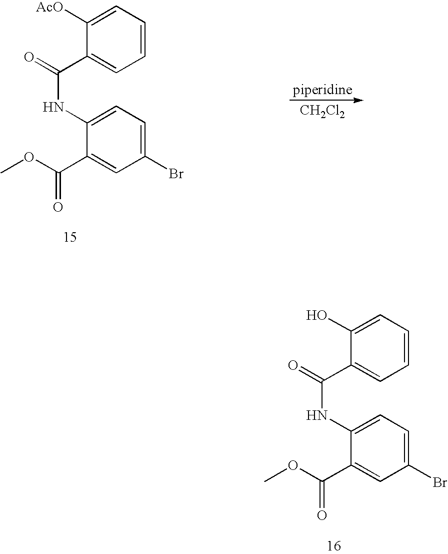

Synthesis of Phenol 16. To a 50-mL round bottom flask was added o-acetoxy methyl ester 15 (1.0 g, 2.9 mmol), CH₂Cl₂ (10 mL) and piperidine (2.0 mL of a 2 M solution in CH₂Cl₂, 4.0 mmol). After the reaction mixture was stirred for 3 h, the solvent was removed and the crude residue dried under high vacuum overnight. The residue was then dissolved in CH₂Cl₂ and acid chloride resin (2.0 g, 2.1 mrnmol) was added to scavenge excess piperidine. The mixture was stirred for 4 h, filtered, and concentrated to afford 0.54 g (60%) of phenol 16 as a white solid: ¹H NMR (DMSO-d₆) δ 3.90 (s, 3H), 6.98 (t, J=7.6, 1H), 7.02 (d, J=7.6, 1H), 7.44 (dt, J=1.8, 8.2, 1H), 7.82 (dd, J=2.5, 9.0, 1H), 7.93 (dd, J=1.8, 7.9, 1H), 8.08 (d, J=2.5, 1H), 8.60 (d, J=9.0, 1H).

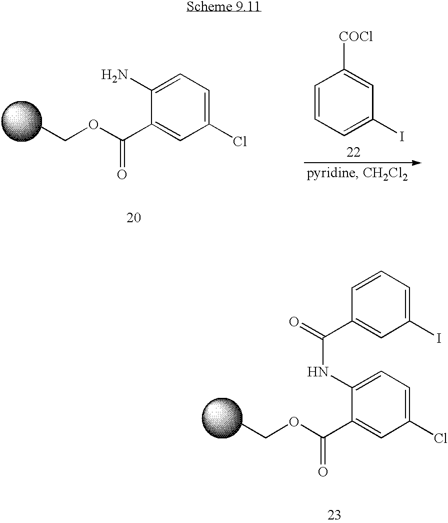

Resin-Bound m-Iodo Benzamide 23. Acid chloride 22 was redissolved in CH₂Cl₂ (30 mL) and added to resin-bound 5-chloroanthranilic acid (20, 3 g, 1.06 mmol/g loading, 3.18 mmol) swollen with pyridine (30 mL) in a 500-mL serum flask equipped with an overhead stirrer. The flask was purged with nitrogen and the resin stirred for 16 h. The resin was filtered from the reaction mixture and washed with alternating CH₃CN and CH₂Cl₂ washes (8×300 mL) to afford 23.

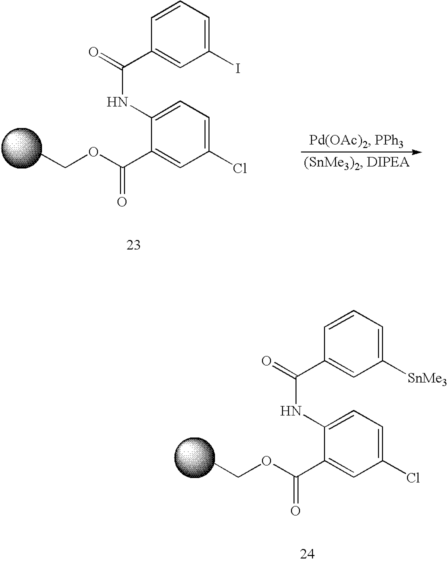

Resin-Bound Stannylate 24. To a CH₂Cl₂ slurry of m-iodo resin (23, 1 g, 1.06 mmol/g) in a 250-mL serum flask was added 1 mL of the following solutions; palladium acetate (0.0022 g/1 mL, 0.01 mmol, 0.1 equiv,), triphenyl phosphine (0.0065 g/mL, 0.025 mmol, 0.25 equiv), DIPEA (0.0065 g/mL, 0.05 mmol, 0.5 equiv) in DMF. Hexamethyl ditin (0.065 g, 0.2 mmol, 2.0 equiv) was added to the flask, which was then purged with nitrogen and heated to 60° C. for 18 h. The reaction mixture was drained and the resin washed with alternating DMF, CH₃CN and CH₂Cl₂ (10×150 mL) to yield 24 as a dark brown resin.

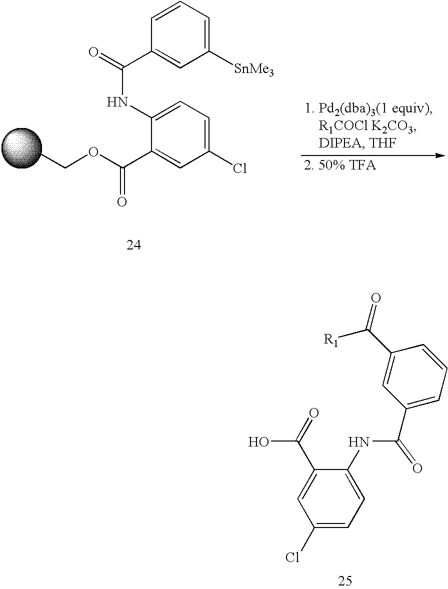

Resin Bound Library of Aryl Ketones. Hexamethyl ditin derivatized Wang resin (24, 24 mg, 24 μmol) was added as an isopycnic solution (degassed THF) to an array of 1-dram vials arranged in a 96-well format. Tris(dibenzylidene acetone) dipalladium (0) (22 mg, 24 ,mol, 1.0 equiv) was added to each vial (in a solution of degassed THF). DIPEA (20 μL) was added to each vial followed by K₂CO₃ (10 mg) and degassed THF (0.5 mL). The vials were capped and shaken. The vials were uncapped and the acid chloride diversity elements⁷ (10 equiv) were then added, the vials purged with nitrogen for 5 sec, capped, shaken and heated 60° C. for 20 h. After the reactions cooled to room temperature, the resin was transferred to a 96-well polypropylene fritted plate. The resin was washed (CH₃CN, DMF, CH₃CN, DMF, CH₃CN, DMF, H₂O, THF, H₂O, THF, H₂O, THF, CH₃CN, CH₂Cl₂, CH₃CN, CH₂Cl₂, CH₃CN, CH₂Cl₂, CH₂Cl₂, CH₂Cl₂, 250 μL each wash) and the plate inserted into a solid phase reaction block. A solution of 50% TFA in CH₂Cl₂ (600 μL) was added to the plate. The plate was capped and spun on an overhead rotisserie for 3 h. The crude aryl ketones (25) were then drained into a 96-well collection plate, concentrated to dryness, and analyzed by HPLC/MS.

Purification Procedures

Liquid-liquid extraction (basic). To a 96-well plate of crude samples was added methyl ethyl ketone (MEK, 500 μL) and 2 N NaOH (500 μL). The plates were capped and shaken. After the plates were uncapped, the organic layer was separated from the aqueous layer.

Liquid-liquid extraction (acidic). The aqueous layer of the above extraction was treated with 6 N HCl (500 μL) and extracted with MEK (1 mL). The plates were capped, shaken, and the organic layer was separated from the aqueous layer.

Hydromatrix® extraction (AMRI SEC-C-44). A set of 2-mL square-well plates were filled with Hydromatrix® and washed with MEK and CH₂Cl₂ (500 μL/well). The plates were then placed in a vacuum oven (T=35° C.) overnight. After cooling, the Hydromatrix® was treated with 2 N HCl (600 μL)⁷ and the plates were stacked. The crude library samples were dissolved in MEK and pipetted onto the columns. MEK was used to elute the compounds, and several 2-mL fractions were collected.

Crystallization

After treatment with Hydromatrix®, several compounds crystallized out of the 50% MeOH/MEK solution. The liquid was removed from the well, and the solid dissolved in DMSO (250 μL) and transferred to a Marsh tube.

HPLC analysis method. The purity of the library was determined from the relative peak area of the UV absorbance. The identity of the compound was determined by MS confirmation of the molecular weight. The samples from this library were best prepared from DMSO solutions of the crude compounds. To a 96-well LC/MS plate was added ca 30 μL of DMSO solution (solution concentration was typically ca 30 mM). DMSO (ca 50 μL) and MeCN (ca 750 μL) were then used to dilute the samples.

HPLC Conditions

| | |
|---|---|
| Column: | Zorbax SB-C18 (4.6 × 75 mm, 3.5 microns) |
| Gradient: | A solvent: 100% MeCN (0.075% HCO₂H), |
| | B solvent: 100% H₂0 (0.075% HCO₂H) |
| Flow: | 2 mL/min |
| Detection wavelength: | 220 nm (UV) |
| Autosampler: | Gilson 215 Liquid Handler |
| Pump: | Shimadzu LC-10AD VP |
| Detector: | Shimadzu UV-VIS Detector SPD-10A VP |
| Injection volume: | 40 μL |
| Mass Spectrometer: | PESCIEX API 150EX |

TABLE 1
Gradient Profile

| Time | |
|---|---|
| (min) | % B |
| 0 | 75 |
| 4.5 | 10 |
| 7.0 | 10 |
| 9.0 | 75 |

Preparation of Benzioic Acid Derivatives for Library Synthesis

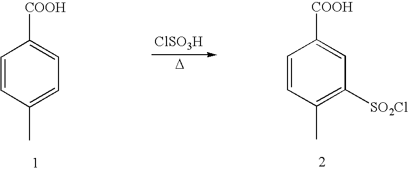

Chlorosulfonic acid (50 mL, 752 mmol) was added to a 250-mL round-bottom flask and cooled to 0° C. in the presence of nitrogen. p-Toluic acid (1, 10 g, 73.7 mmol) was added in small portions over 5 min to give a yellow solution. The solution was warmed to room temperature and heated to 100° C. overnight. The reaction mixture was then cooled to room temperature and poured over ice (ca 750 g). The resulting precipitate was filtered, washed with water and dried in a vacuum oven at 70° C. for 8 h to afford 14.38 g (83%) of 2 as an off-white solid: ¹H NMR (DMSO-d₆) δ 2.60 (s, 3H), 7.28 (d, J=7.9 Hz, 1H), 7.79 (d, J=7.8 Hz, 1H), 8.31 (s, lH), 13.87 (br s, 1H).

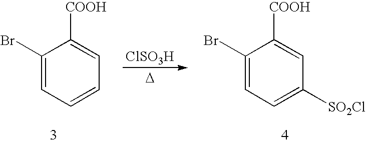

To a 250-mL round-bottom flask cooled to 0° C. under nitrogen was added chlorosulfonic acid (50 mL, 752 mmol), followed by o-bromobenzoic acid (3, 10.0 g, 49.7 mmol) in small portions over 2 min to give a brownish solution. The solution was warmed to room temperature and heated to 115° C. overnight. The reaction mixture was then cooled to room temperature and poured over ice (ca 750 g).¹ The resulting precipitate was filtered, washed with water and dried in a vacuum oven at 80° C. for 7 h to afford 12.81 g (86%) of 4 as an off-white solid: ¹H NMR (DMSO-d₆) δ 7.75 (d, J=10.1 Hz, 1H), 7.65 (d, J=10.1 Hz, 1H), 8.46 (s, 1H), 13.96 (br s, 1H).

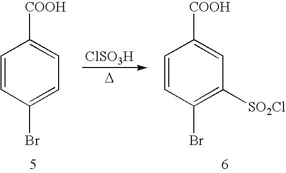

Chlorosulfonic acid (50 mL 752 mmol) was added to a 250-mL round-bottom flask and cooled to 0° C. in the presence of nitrogen. p-Bromobenzoic acid (5, 10.0 g, 49.7 mmol) was added in small portions over 2 min to give a brownish solution. The solution was warmed to room temperature and heated to 145° C. overnight. The reaction mixture was then cooled to room temperature and poured over ice (ca 750 g).¹ The resulting precipitate was filtered, washed with water and dried in a vacuum oven at 80° C. for 7 h to afford 13.21 g (89%) of 6 as a tan solid: ¹H NMR (DMSO-d₆) δ 7.60 (dd, J=2.1, 8.3 Hz, 1H), 7.69 (d, J=8.2 Hz, 1H), 8.31 (d, J=2.1 Hz, 1H), 14.05 (br s, 1H).

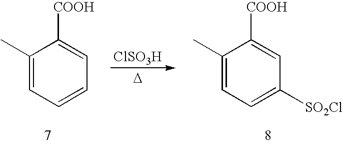

Chlorosulfonic acid (50 mL, 752 mmol) was added to a 250-mL round-bottom flask and cooled to 0° C. in the presence of nitrogen. o-Toluic acid (7, 10.0 g, 73.4 mmol) was added in small portions over 2 min to give a brownish solution. The solution was warmed to room temperature and heated to 145° C. overnight. The reaction mixture was then cooled to room temperature and poured over ice (ca 750 g).¹ The resulting precipitate was filtered, washed with water and dried in a vacuum oven at 80° C. for 7 h to afford 15.53 g (90%) of 8 as an off-white solid: ¹H NMR (DMSO-d₆) δ 2.53 (s, 3H), 7.26 (d, J=7.9 Hz, 1H), 7.63 (d, J=7.9 Hz, 1H), 8.07 (s, 1H), 13.60 (br s, 1H).

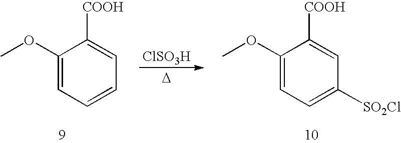

Chlorosulfonic acid (50 mL, 752 mmol) was added to a 250-mL round-bottom flask and cooled to 0° C. in the presence of nitrogen. p-Anisic acid (9, 10.0 g, 73.4 mmol) was added in small portions over 2 min to give a yellow solution. The solution was warmed to room temperature and heated to 63° C. for 1 h. The reaction mixture was then cooled to room temperature and poured over ice (ca 750 g).¹ The resulting precipitate was filtered, washed with water and dried in a vacuum oven at 70° C. for 12 h to afford 14.62 g (85%) of 10 as a white solid: ¹H NMR (DMSO-d₆) δ 3.84 (s, 3H), 7.06 (d, J=8.7 Hz, 1H), 7.70 (dd, J=2.3, 8.7 Hz, 1H), 8.31 (d, J=2.3 Hz, 1H), 13.82 (br s, 1 H).

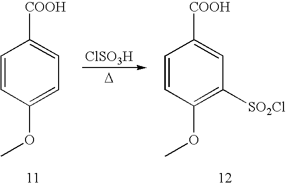

Solid p-anisic acid (11, 10.0 g, 66 mmol) was added to an ice-cooled, 250-mL round-bottom flask containing chlorosulfonic acid (50 mL, 752 mnuol) under nitrogen. The solution was heated at 65° C. for 1 h and turned bright yellow. The reaction mixture was cooled to room temperature and poured over ice (ca 750 g). The resulting precipitate was then filtered, washed with water and dried in a vacuum oven at 70° C. for 8 h to yield 13.18 g (80%) of 12 as a pale yellow solid: ¹H NMR (DMSO-d₆) δ 3.88 (s, 3H), 7.06 (d, J=8.7 Hz, 1H), 7.90 (dd, J=2.4, 8.6 Hz, 1H), 8.31 (d, J=2.3 Hz, 1H), 13.82 (br s, 1 H).
General Procedure for the Conversion of Acids to Acid Chlorides in a Plate Format

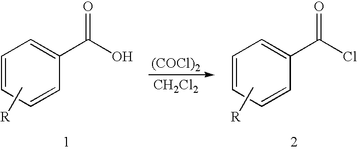

To a plate of 2-mL glass reaction tubes arranged in a standard 96-well format was added the diversity set of carboxylic acids (1, 250 μL, 1 M THF, 250 μmol). The samples were concentrated in a Genevac HT-4 (20% heat with no heat boost for 1 h). A solution of 1% DMF/CH₂Cl₂ (50 μL) was added to the wells, followed by CH₂Cl₂ (250 μL). The carboxylic acid plate was placed in a nitrogen-filled glove bag and oxalyl chloride (125 μL, 2 M CH₂Cl₂, 250 μmol) was added. After the addition of CH₂Cl₂ (250 μL), a capmat with 96 predrilled holes was fitted on the plate. The plate was shaken on an orbital shaker in a N₂ filled glove bag for 6-8 h.
Preparation of Isatioc Anhydride Derivatives

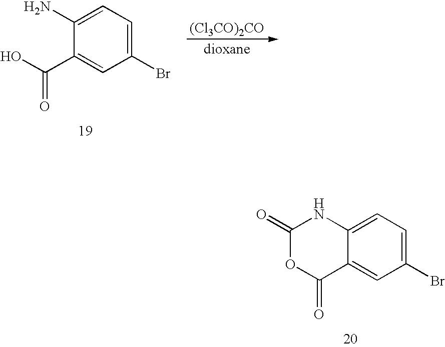

To a dry, 4-L round bottom flask was added 175 g (810 mmol) of 2-amino-5-bromobenzoic acid (19), triphosgene (83 g, 278 mmol), and dioxane (3 L). The suspension was stirred under N₂ and heated to reflux. The reaction was found to be complete by TLC and NMR after stirring at reflux for 3 h, but did not become homogenous at any time. After cooling to room temperature, the reaction was filtered and the precipitate washed with ether. The solid was dried in the vacuum oven at 40° C. to afford 5-bromoisatoic anhydride (20, 151.1 g, 72%) as a white solid: ¹H NMR (DMSO-d₆) δ 7.29 (d, J=8.7, 1H), 7.91 (dd, J=2.5, 8.7, 1H), 8.09 (d, J=2.3, 1H).

Sodium cyanoborohydride (4.88 g, 77.8 mmol) was added to a solution of 6-chloroindoline (5.9 g, 38.9 mmol) in acetic acid (100 mL). Gas evolution was evident at the beginning of the reaction. After stirring for 10 h, the solution was diluted with water (100 mL) and 6 N NaOH was added until the pH of the reaction mixture was 12-13. The resulting mixture was extracted with CH₂Cl₂ (3×200 mL), and the combined organic layers dried over MgSO₄. Flash column chromatography on silica gel (35% EtOAc/hexanes) yielded 2.3 g (39%) of a clear liquid: ¹H NMR (DMSO-d₆) δ 2.87 (t, J=8.4 Hz, 2H), 3.44 (t, J=8.4 Hz, 2H), 6.45 (d, J=1.8 Hz, 1H), 6.47 (dd, J=1.8, 7.6 Hz, 1H), 6.96 (d, J=7.3 Hz, 1H).

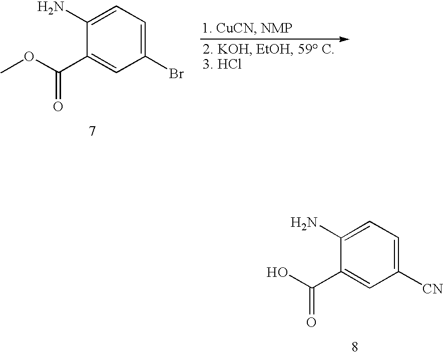

To a 3-L, three-necked, round bottom flask equipped with a reflux condenser was added methyl-2-amino-5-bromobenzoate (7, 125 g, 543 mmol), copper cyanide (56.2 g, 624 mmol), and NMP (1 L). The reaction was heated to 200° C. and stirred for 4 h under nitrogen. The dark brown reaction mixture was allowed to cool and a brown precipitate was formed. The mixture was poured into a 16-L beaker containing sodium cyanide solution (1 kg NaCN in 6 L H₂O) followed by the addition of EtOAc (4 L). The precipitate was dissolved by agitation and the layers were separated. The aqueous layer was extracted with EtOAc (2×1.5 L) and the combined organic layers were washed with 10% NaCN solution (2 L), H₂O (2 L) and then dried over MgSO₄. The light brown solution was concentrated and then dried in a vacuum oven overnight.

The ester was then dissolved in EtOH (2 L) and added to a 3-L round bottom flask followed by KOH solution (96.7 g of KOH in 500 mL H₂O). The reaction mixture was heated to 50° C. and stirred for 2 h. The resultant dark-brown solution was poured into a chilled 2 N HCl solution (1.5 L), creating a yellowish precipitate. The solid was collected on a sintered glass filter frit, washed with cold water, and dried at 35° C. in a vacuum oven overnight to give 64.0 g (73%) of 5-cyanoanthranilic acid: ¹H NMR (DMSO-d₆) δ 7.16 (d, J=8.7, 1 H), 7.79 (dd, J=2.5, 8.7, 1 H), 7.87 (d, J=2.4, 1H).

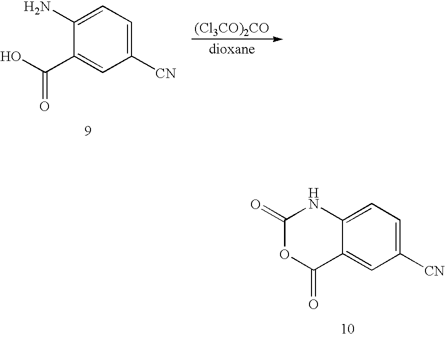

To a dry 4-L round bottom flask was added 5-cyanoanthranilic acid (9, 64 g, 395 mmol), triphosgene (39.4 g, 131 mmol) and dioxane (2 L). The suspension was stirred under N₂ and heated to reflux. The reaction mixture became homogeneous after stirring at reflux for 2 h. As the carbonylation product was formed, white precipitate appeared in the solution. After stirring at reflux for an additional 3 h, the reaction was cooled to room temperature, filtered, and the precipitate washed with ether. The solid was dried in the vacuum oven to afford 5-cyanoisatoic anhydride (10, 51.5 g, 68%) as a pale yellow solid: ¹H NMR (DMSO-d₆) δ 6.86 (d, J=9.3, 1H), 7.55 (dd, J=2.6, 9.0, 1H), 8.04 (d, J=2.4, 1H).

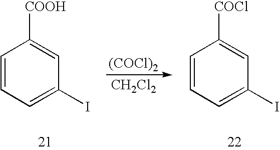

3-lodobenzoyl Chloride. To a suspension of m-iodobenzoic acid (21, 5.0 g, 20.1 mmol) suspended in CH₂Cl₂ (60 mL) was added DMF (2 drops), followed by oxalyl chloride (20.1 mL of a 2 M solution in CH₂Cl₂, 40.2 mmol) under nitrogen atmosphere. After stirring for 18 h, the reaction mixture was nearly homogeneous. Acid chloride 22 was then concentrated, azeotroped with toluene (2×25 mL), and placed on a high vacuum.

### Example 10

### Additional Compounds Useful for Sterilization, Sanitation, Antisepis, and Disenfection

The following compounds may be synthesized using the methodology described above or via methods known in the art.

Compound No., Structure

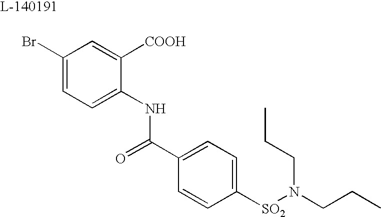

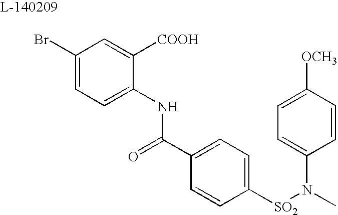

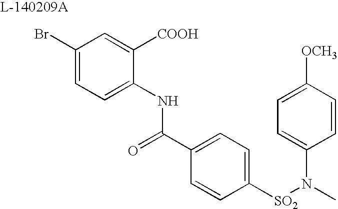

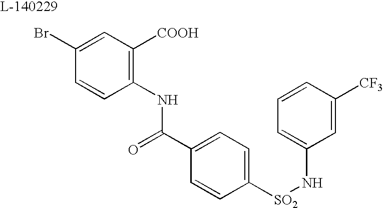

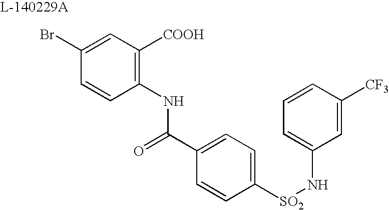

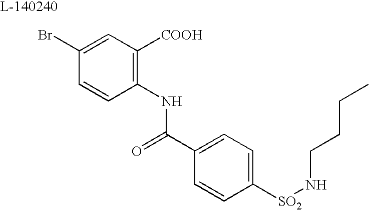

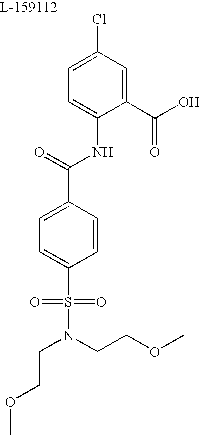

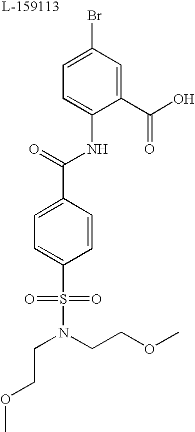

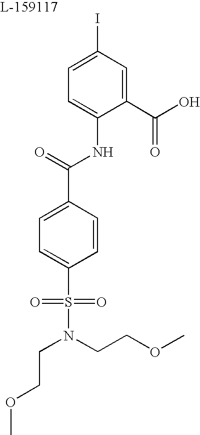

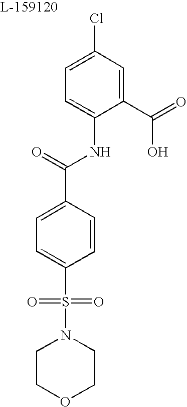

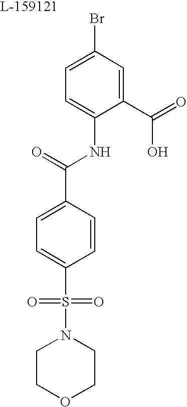

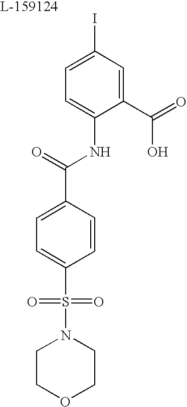

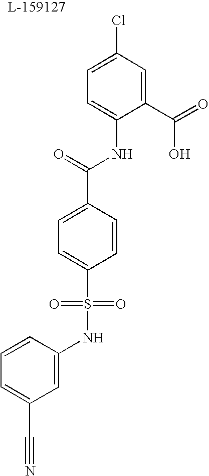

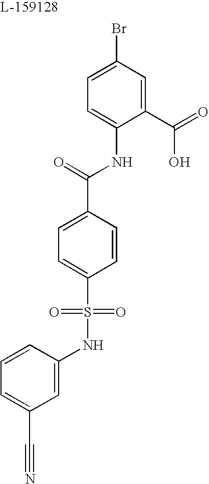

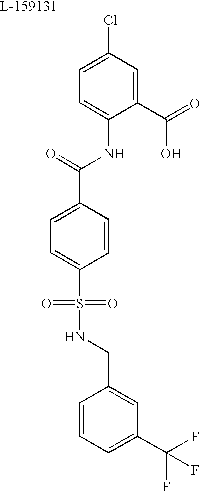

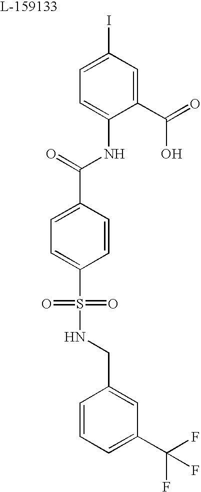

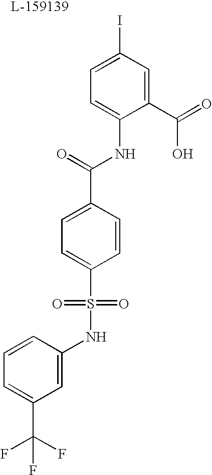

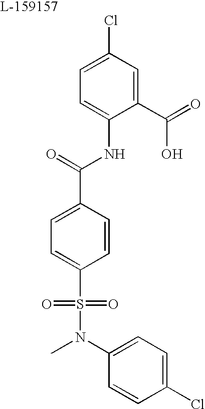

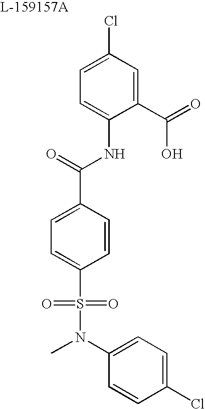

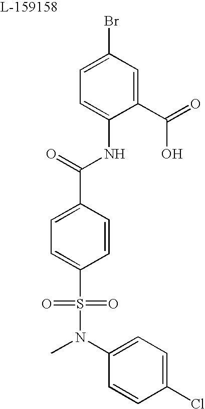

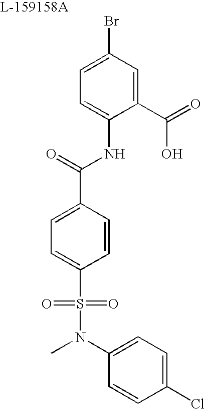

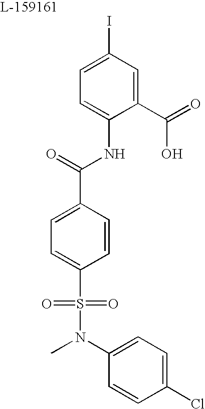

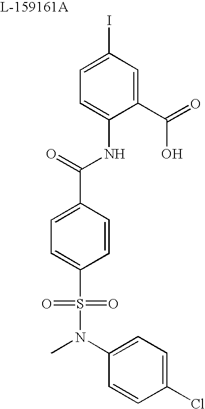

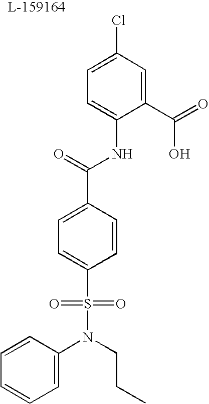

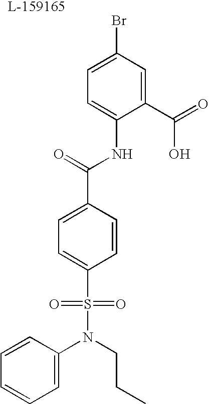

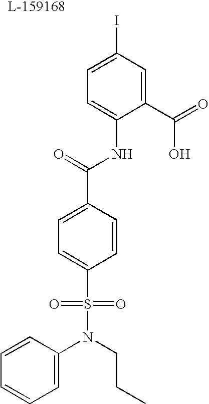

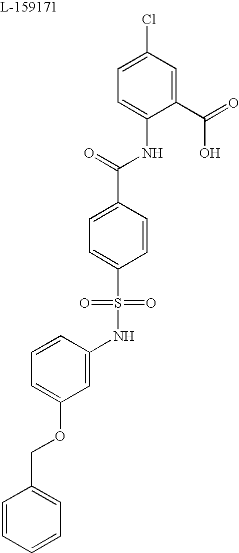

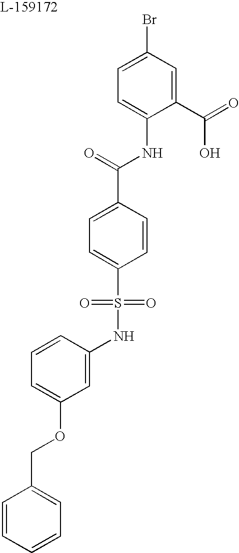

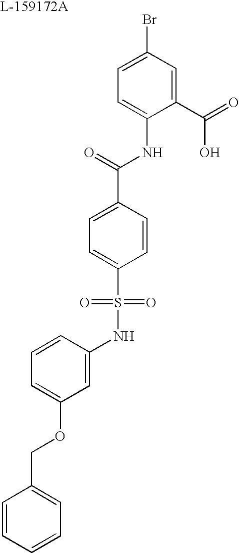

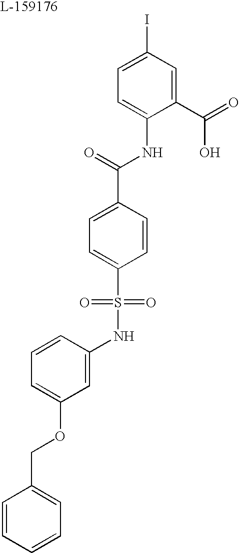

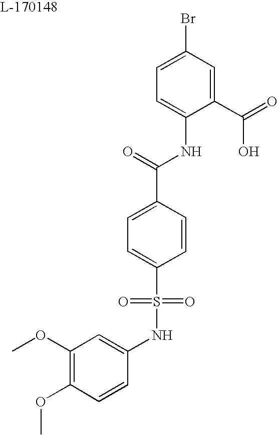

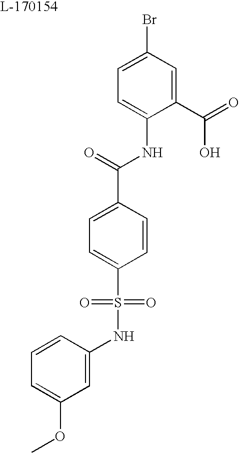

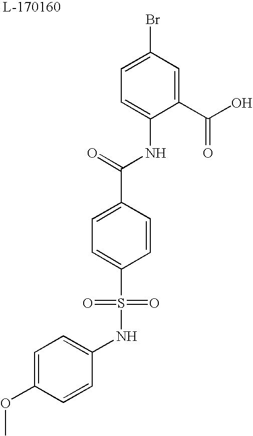

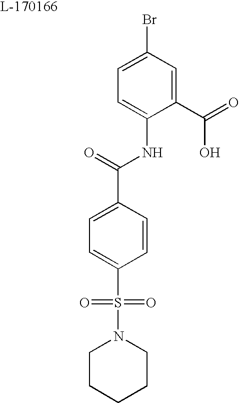

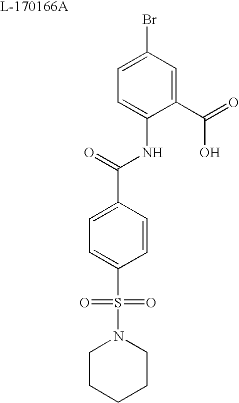

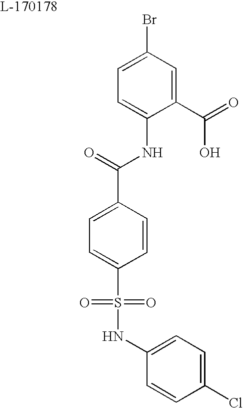

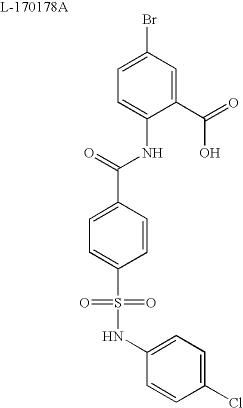

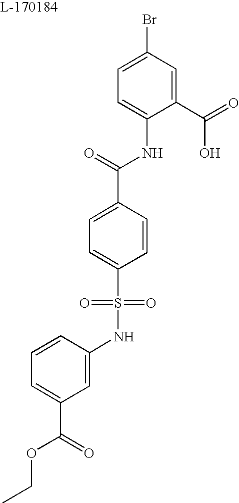

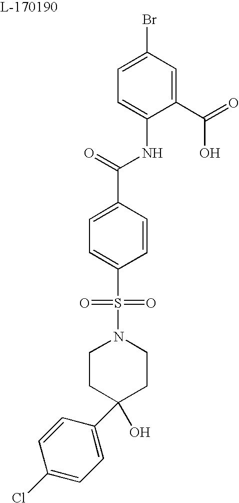

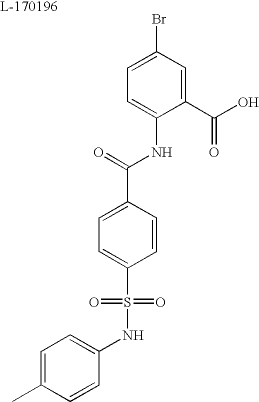

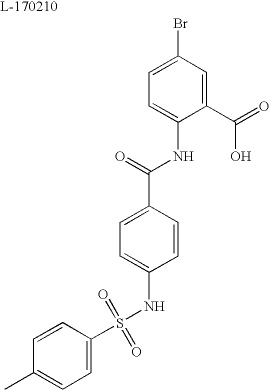

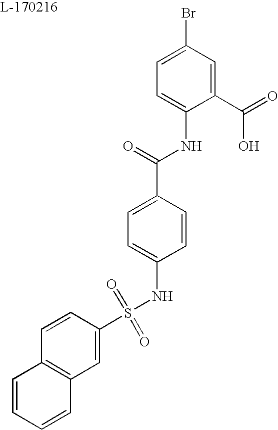

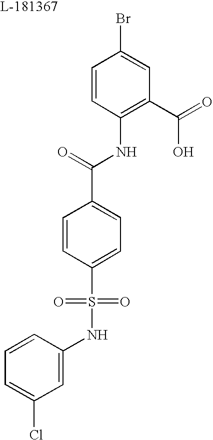

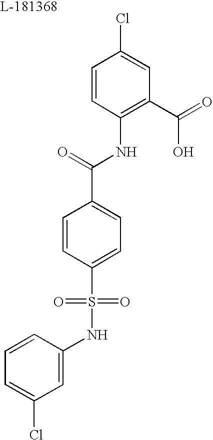

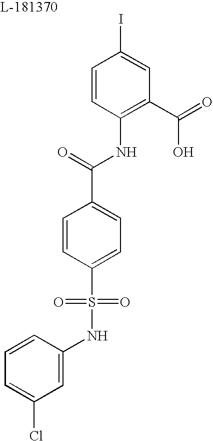

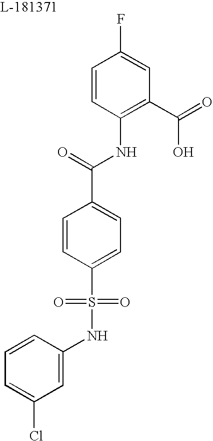

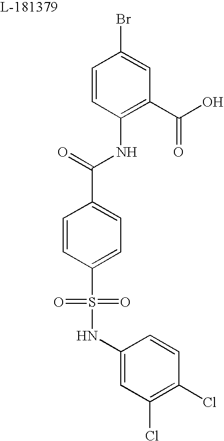

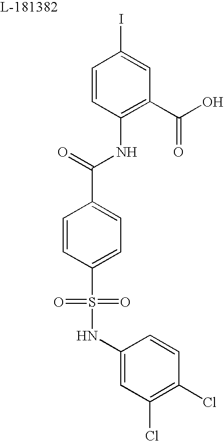

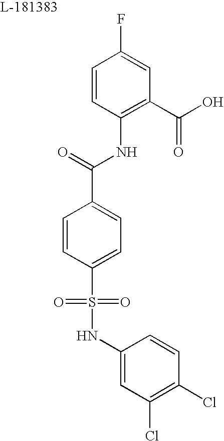

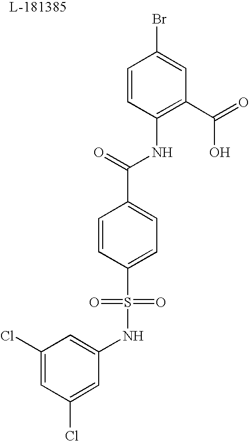

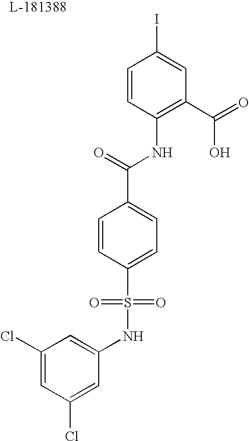

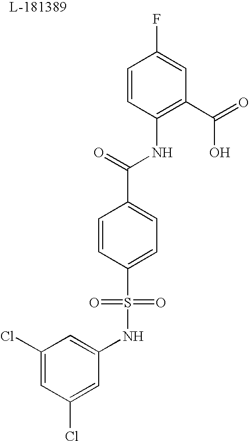

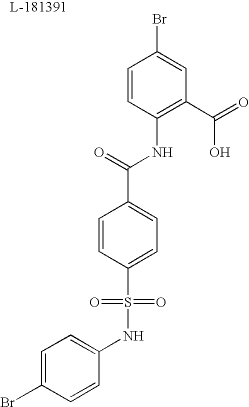

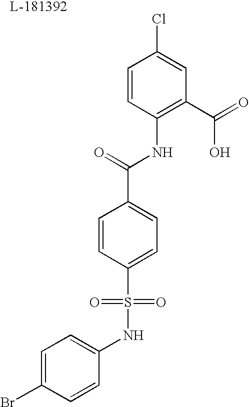

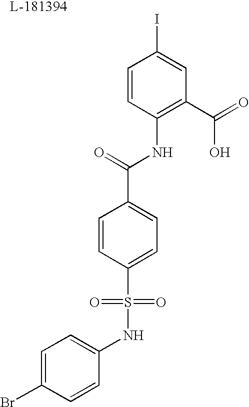

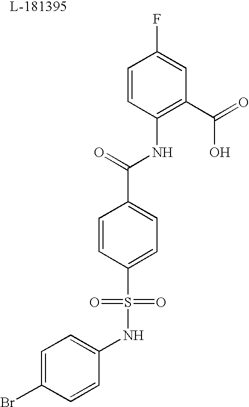

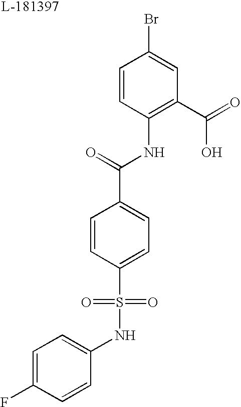

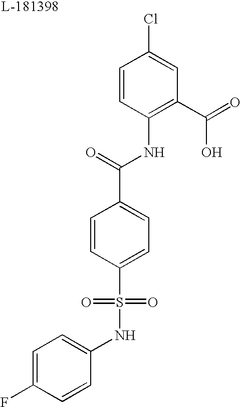

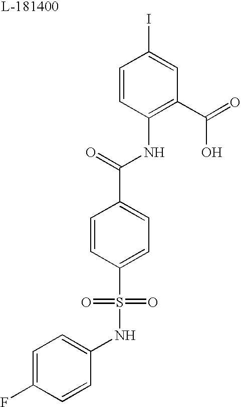

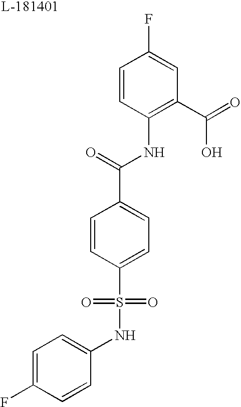

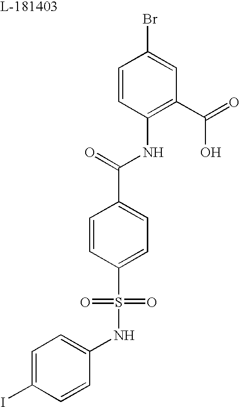

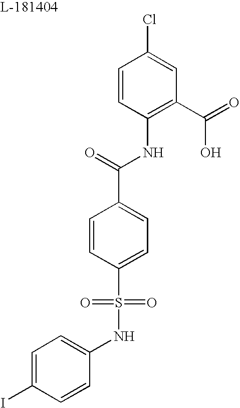

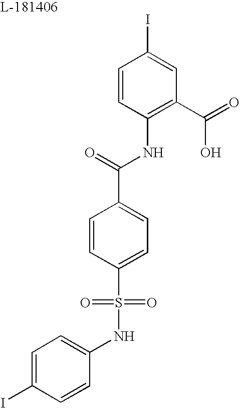

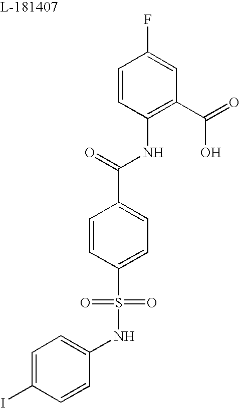

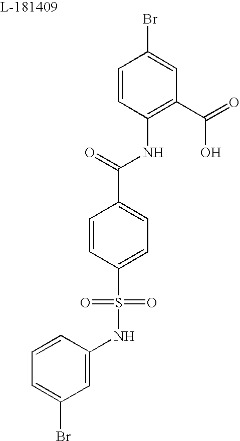

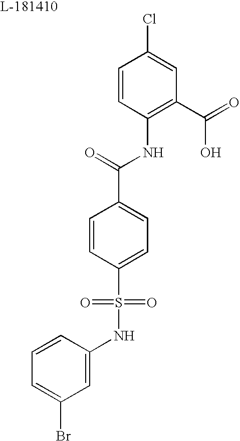

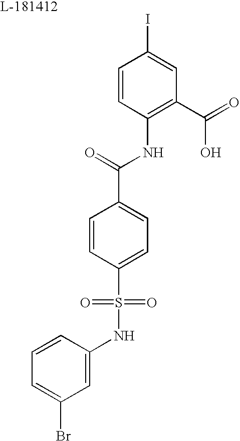

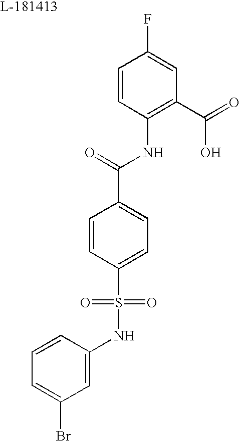

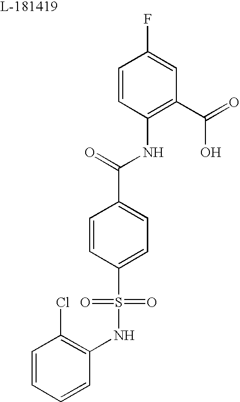

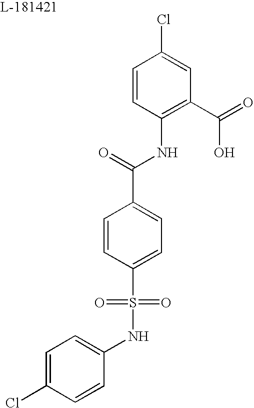

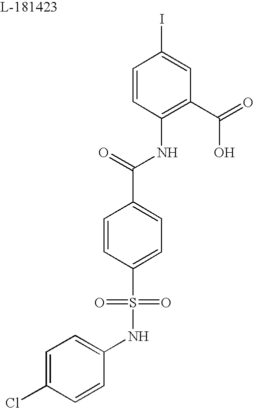

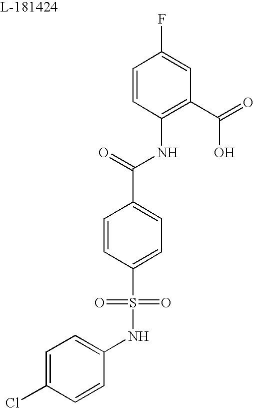

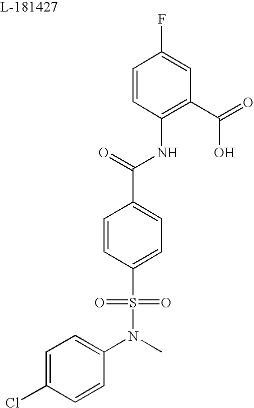

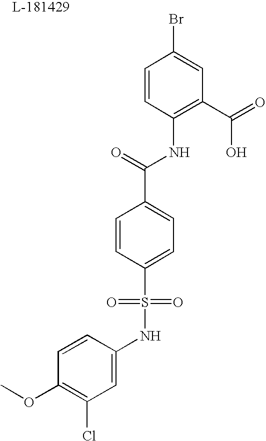

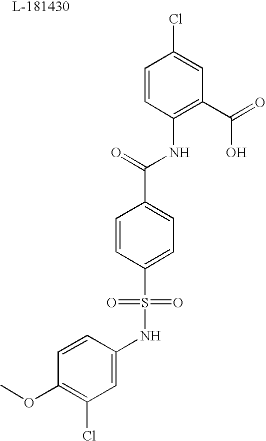

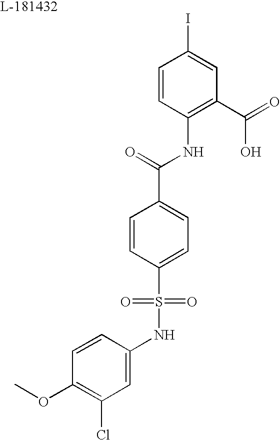

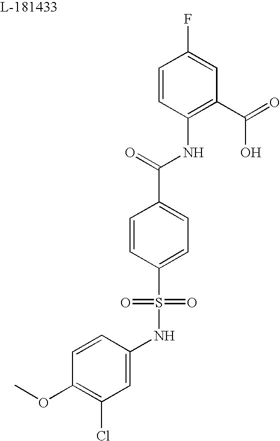

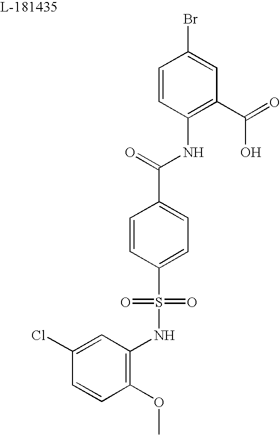

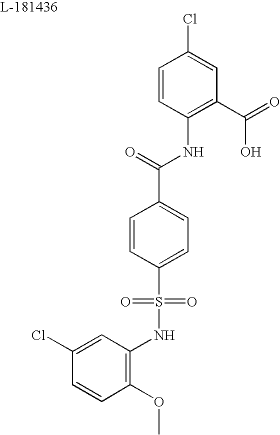

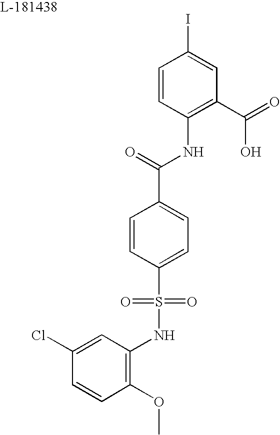

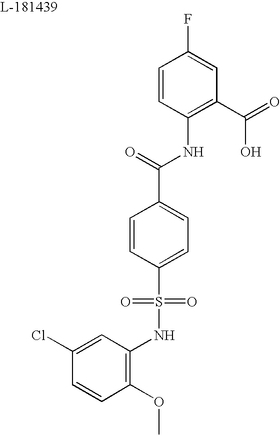

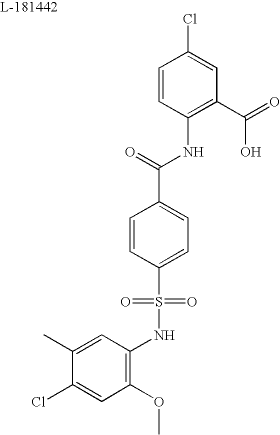

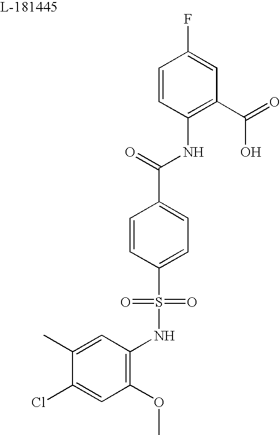

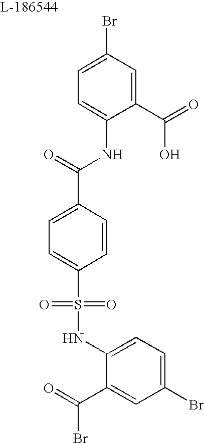

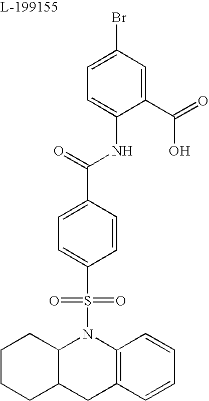

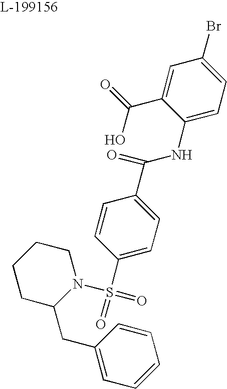

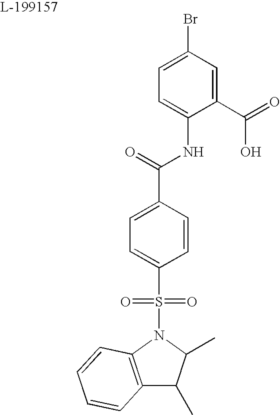

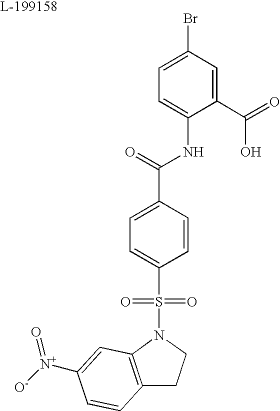

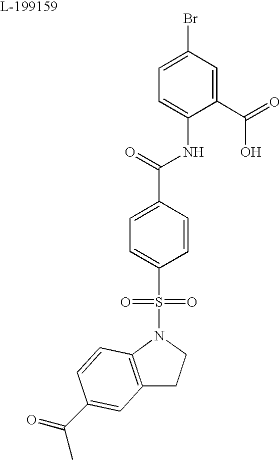

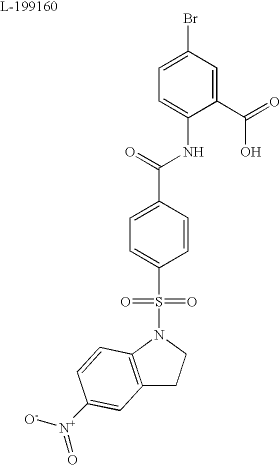

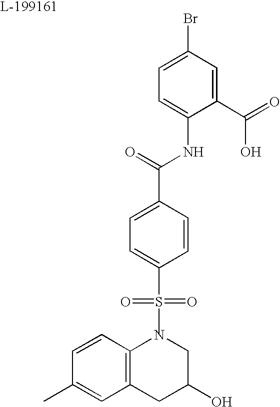

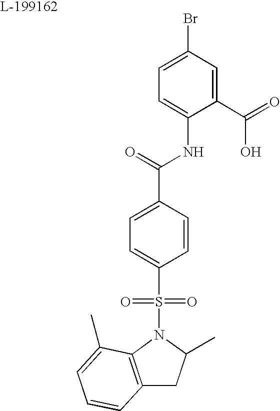

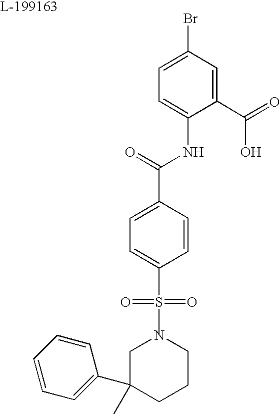

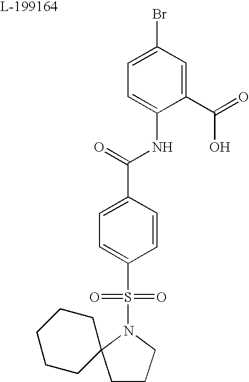

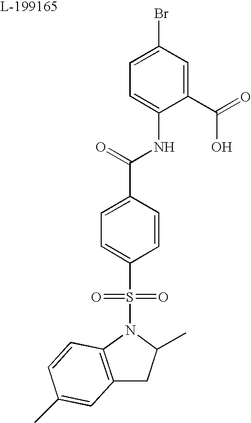

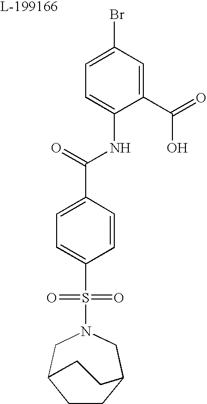

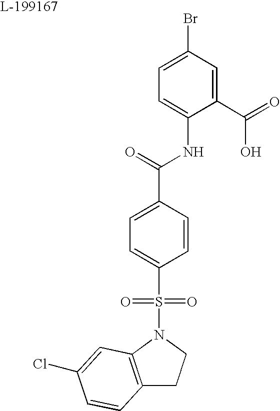

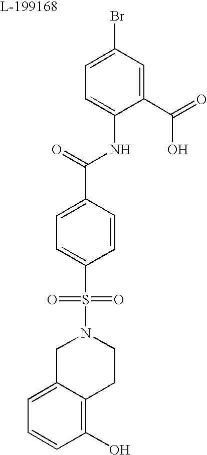

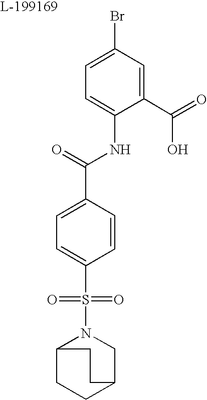

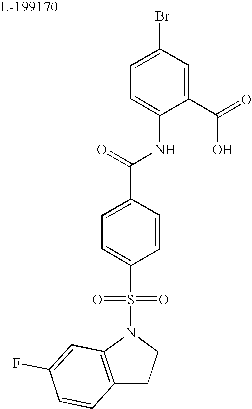

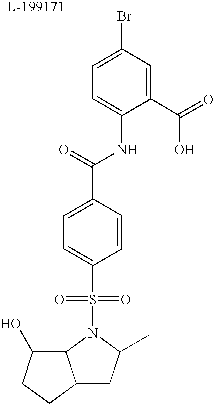

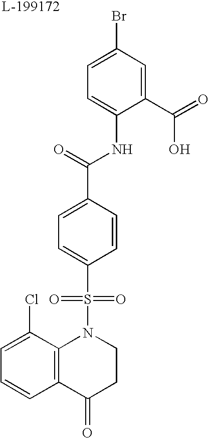

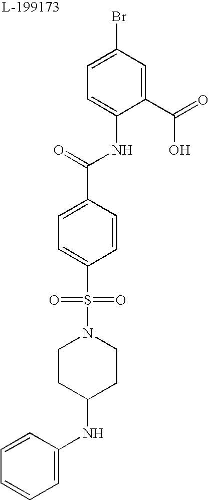

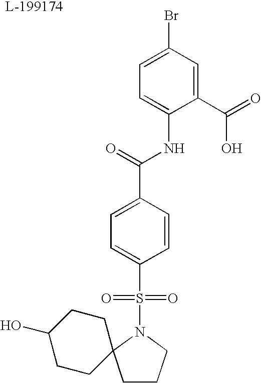

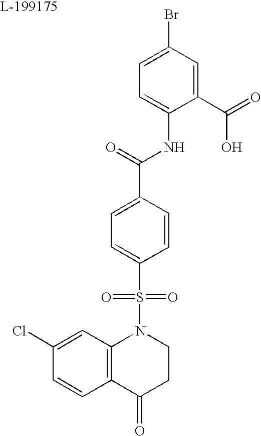

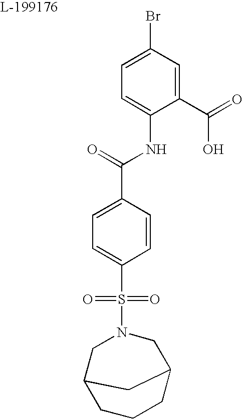

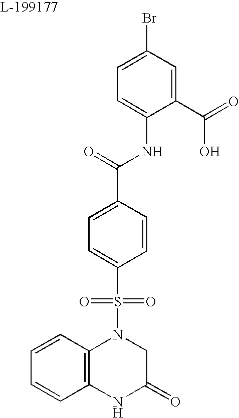

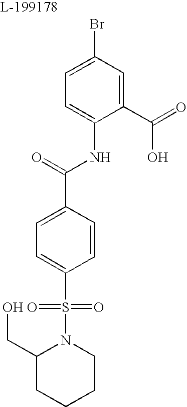

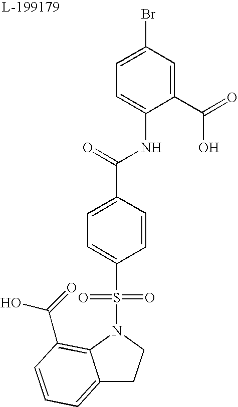

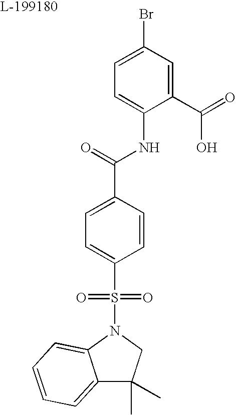

Compound No., Structure

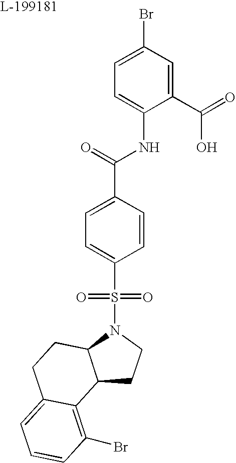

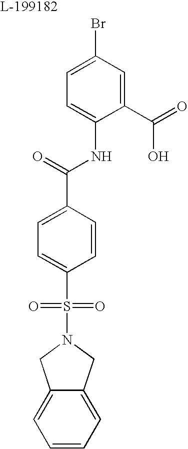

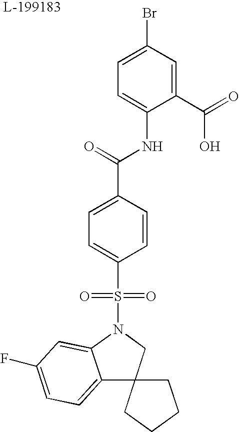

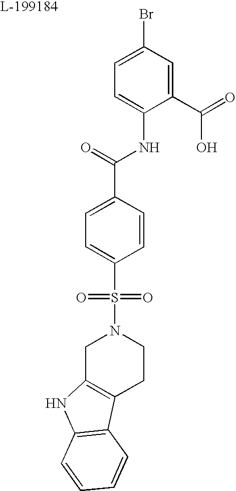

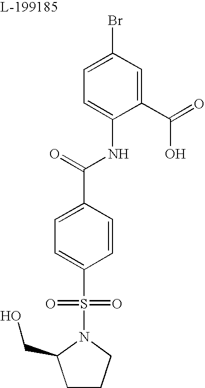

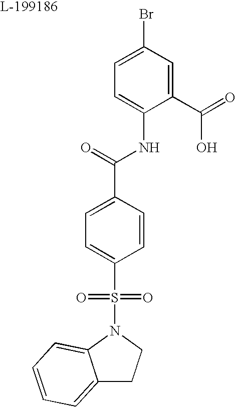

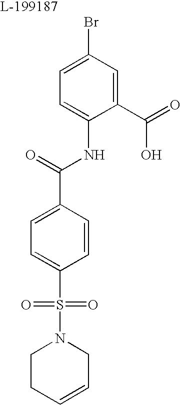

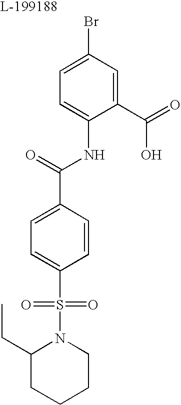

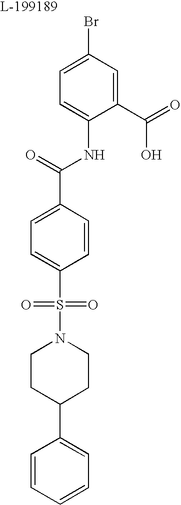

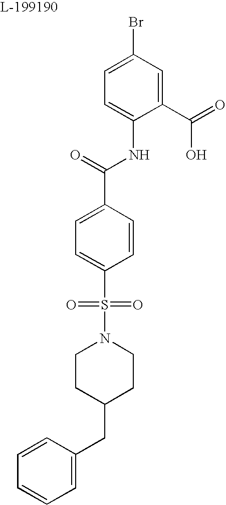

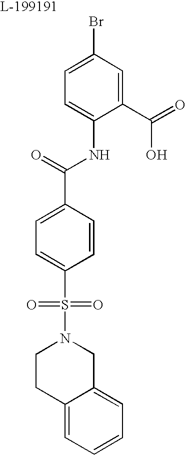

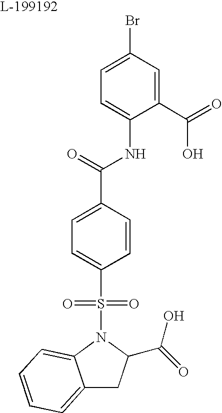

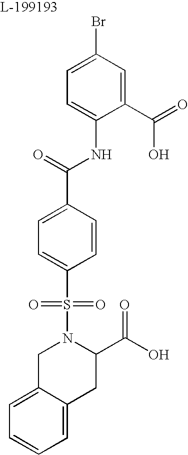

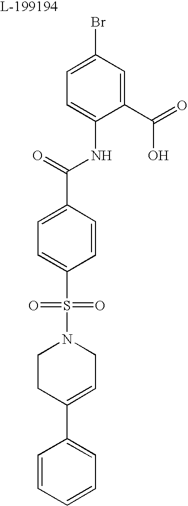

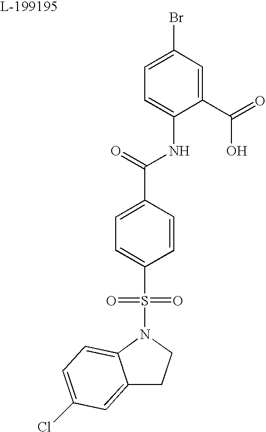

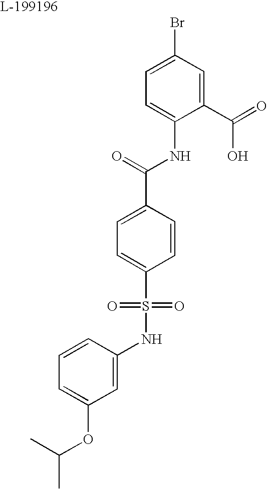

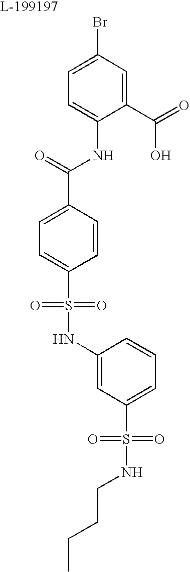

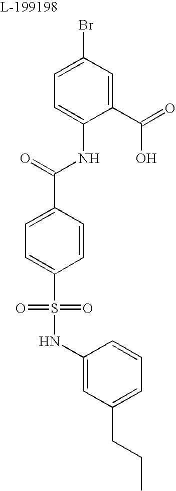

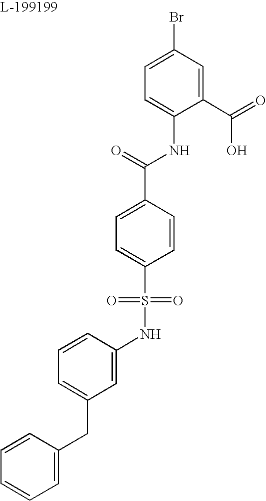

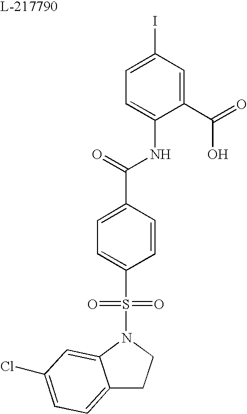

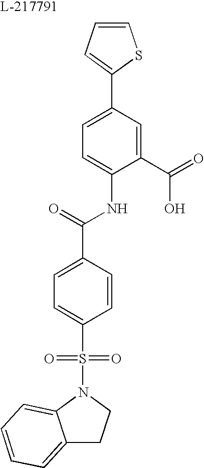

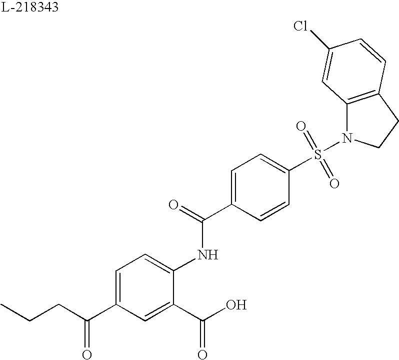

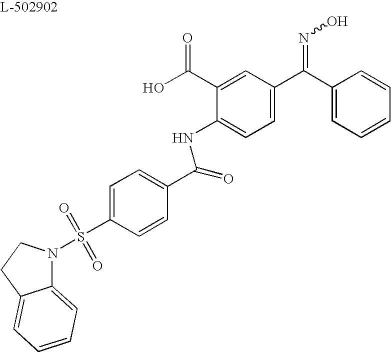

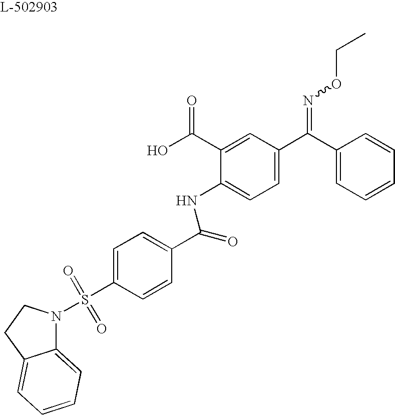

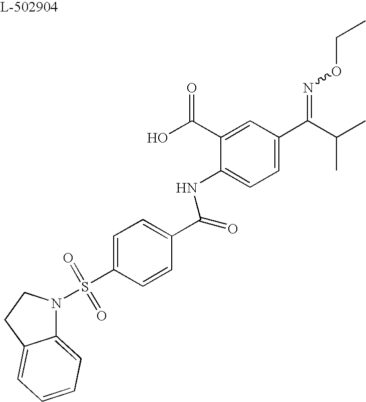

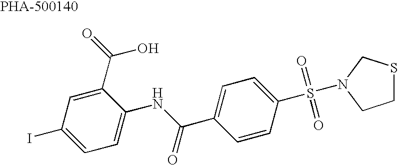

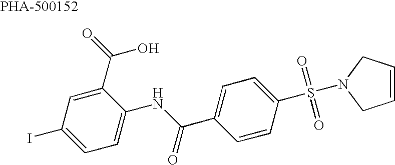

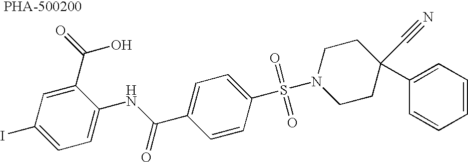

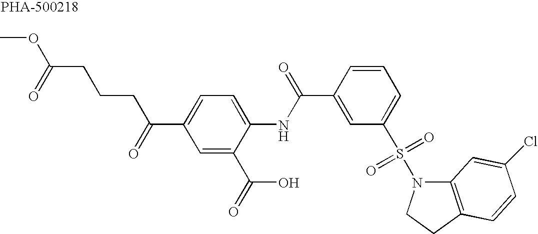

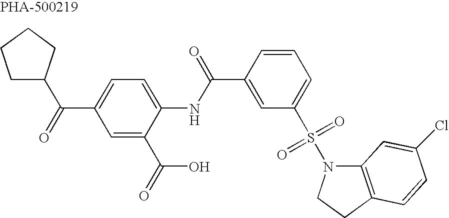

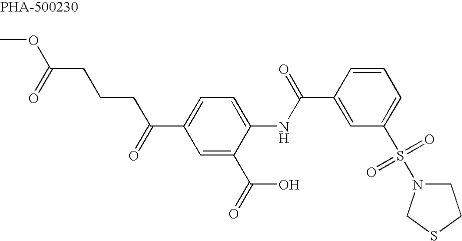

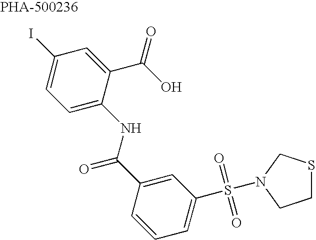

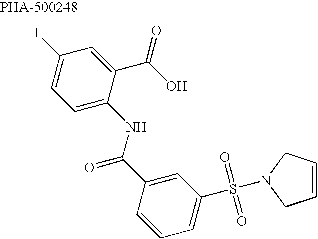

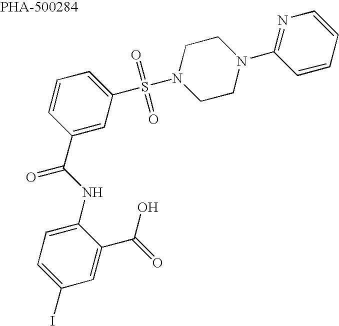

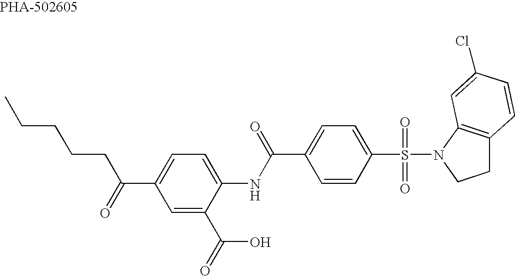

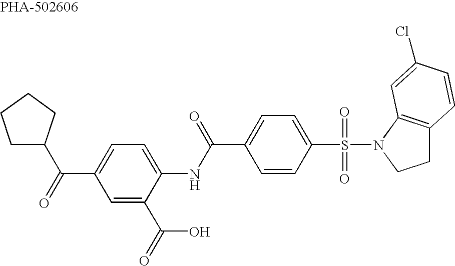

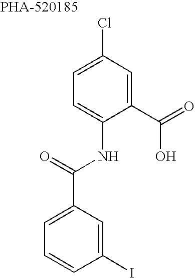

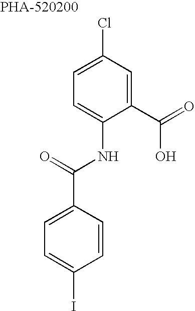

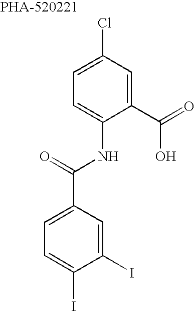

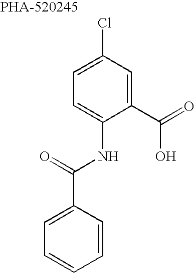

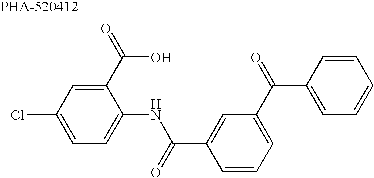

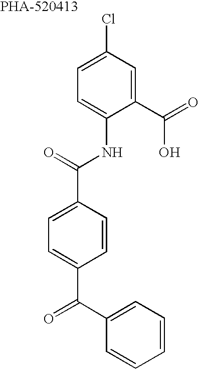

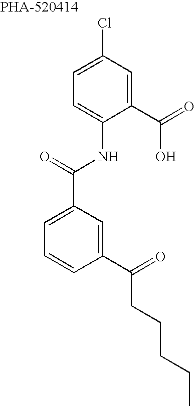

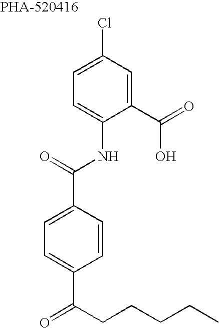

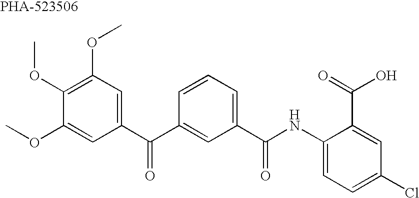

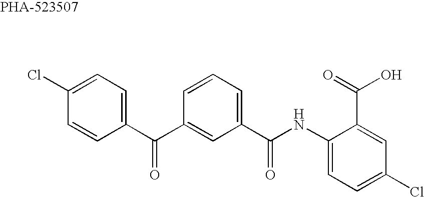

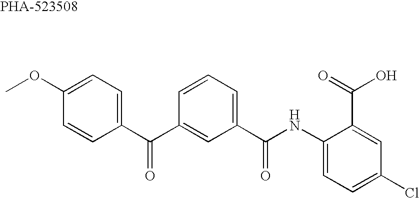

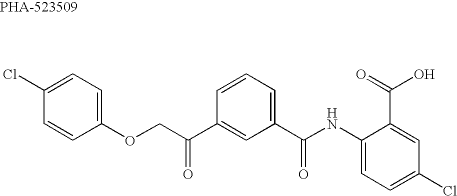

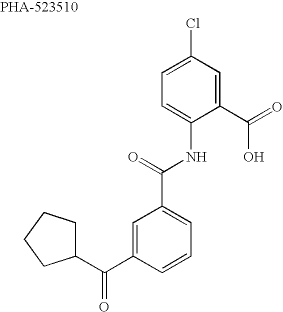

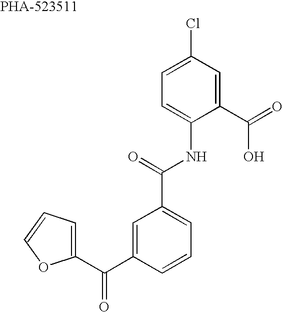

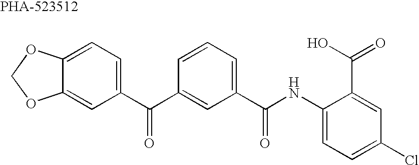

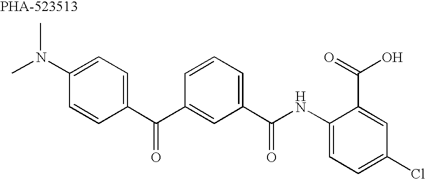

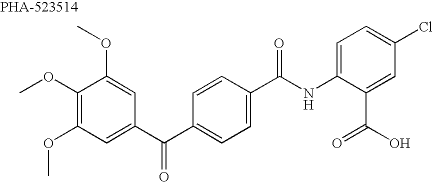

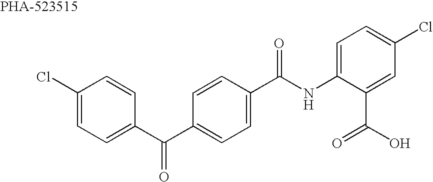

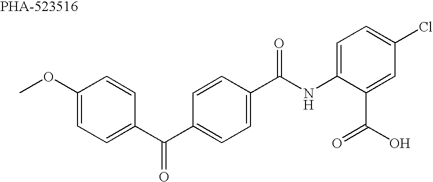

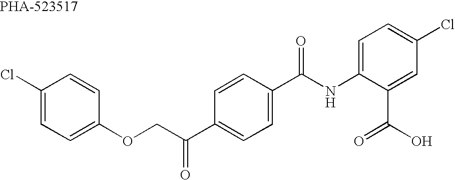

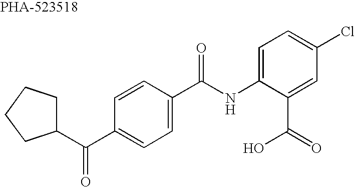

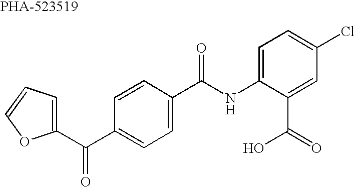

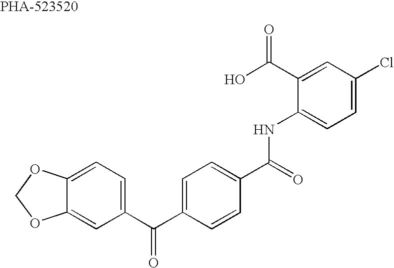

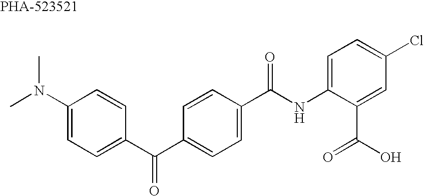

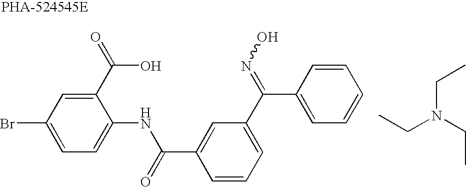

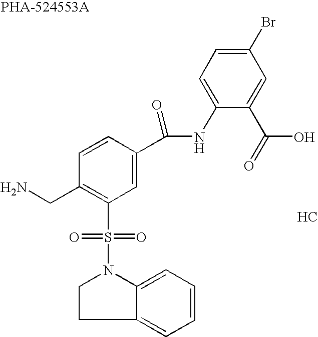

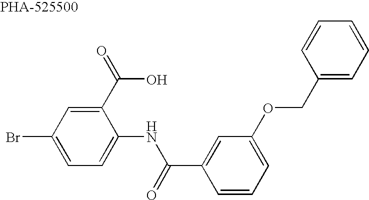

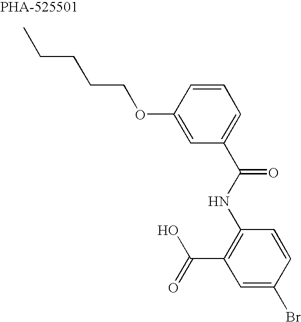

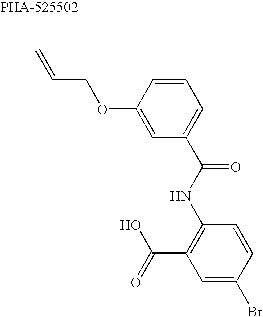

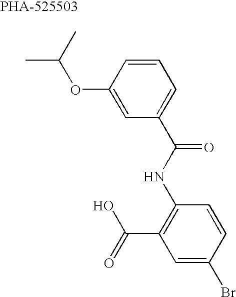

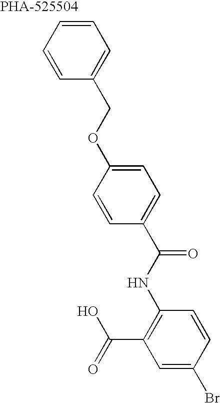

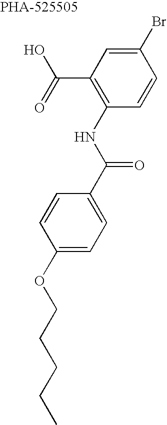

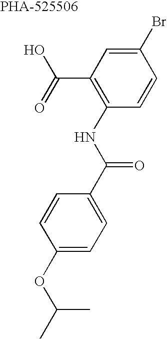

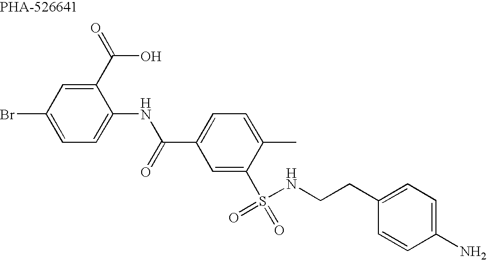

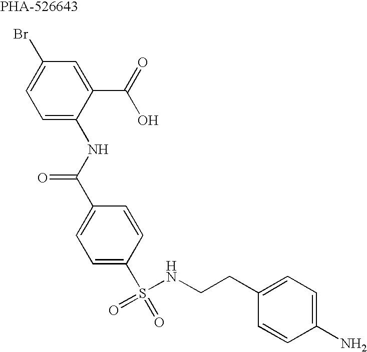

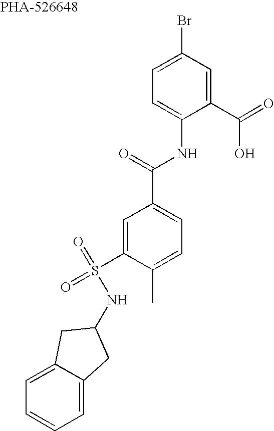

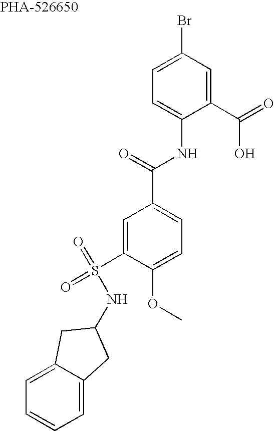

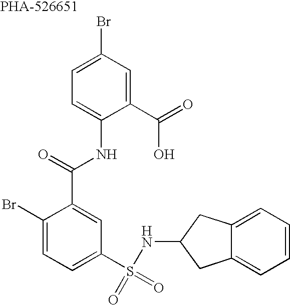

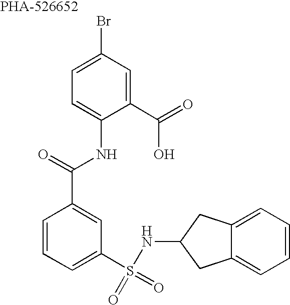

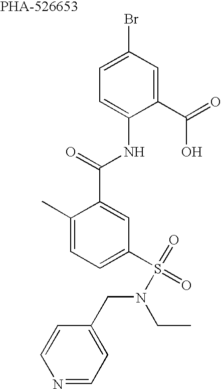

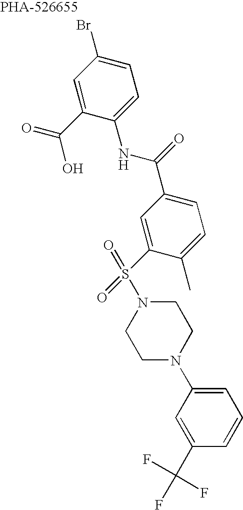

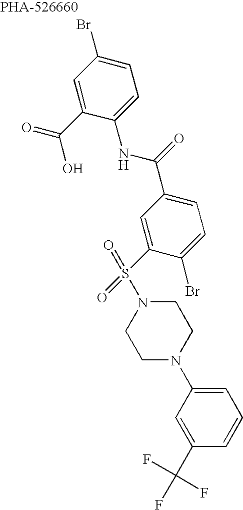

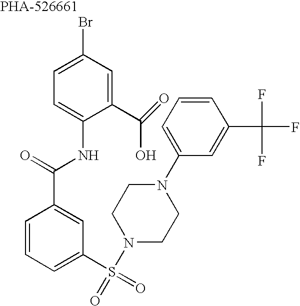

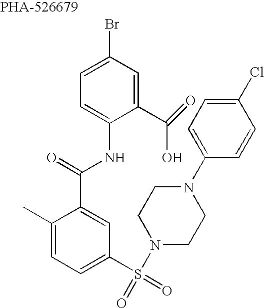

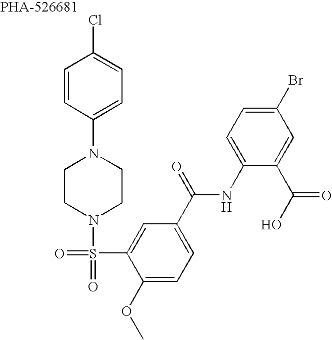

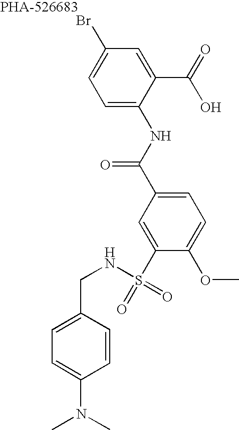

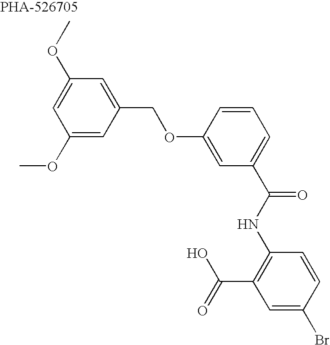

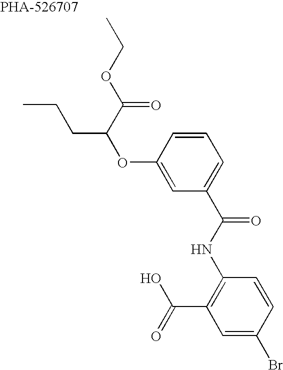

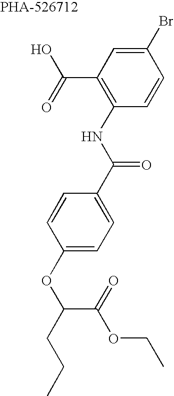

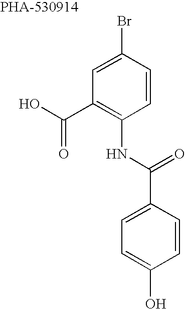

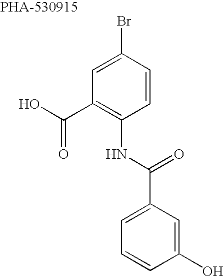

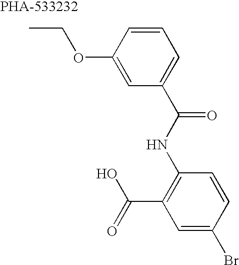

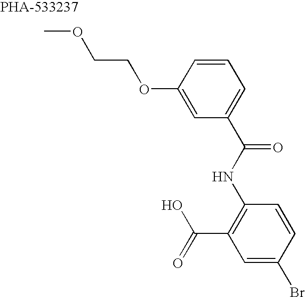

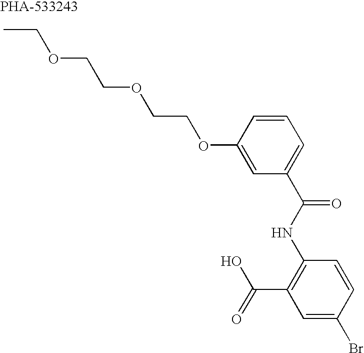

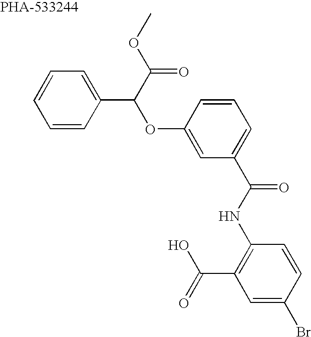

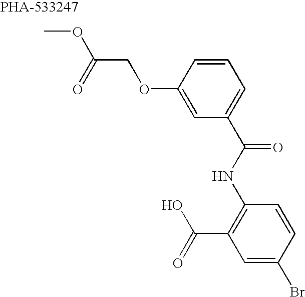

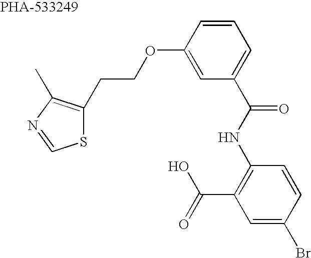

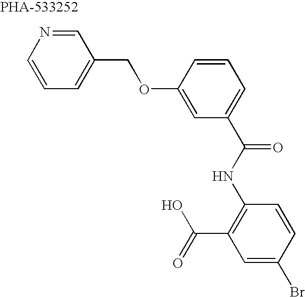

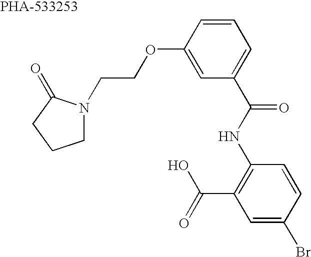

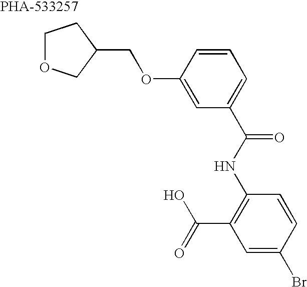

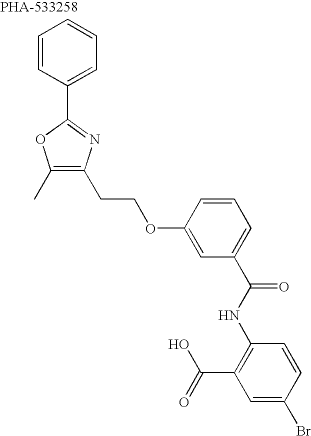

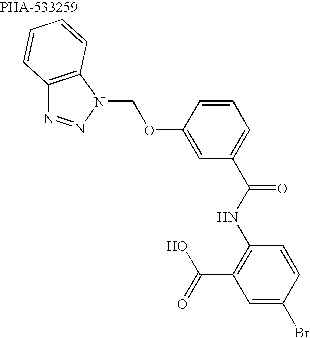

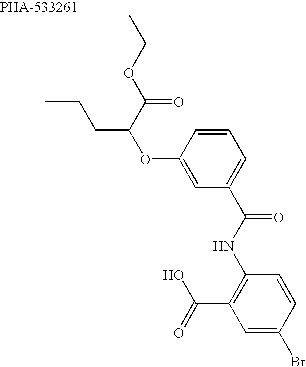

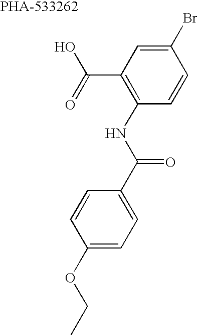

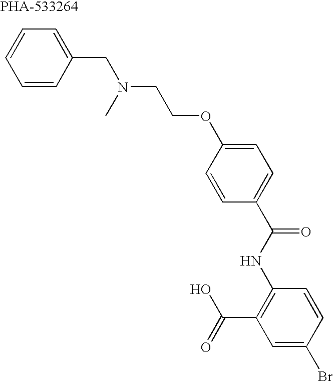

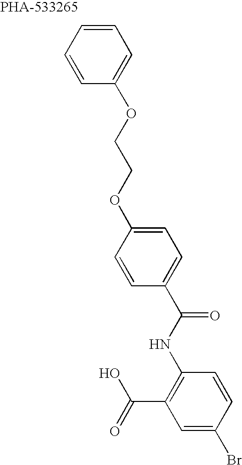

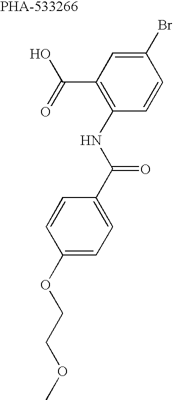

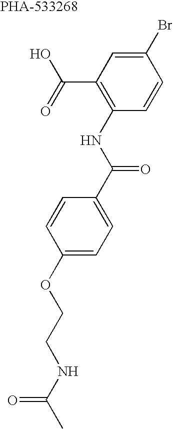

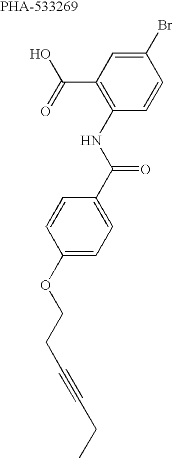

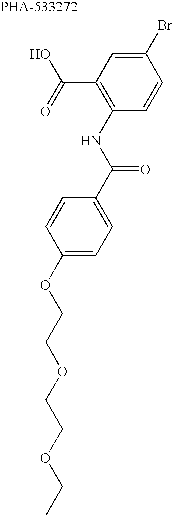

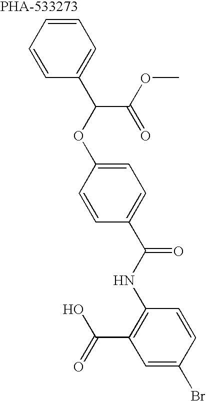

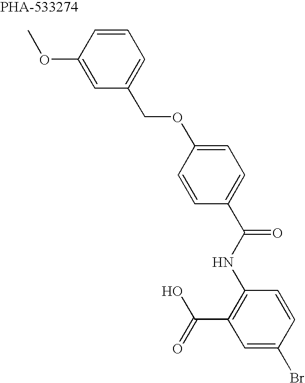

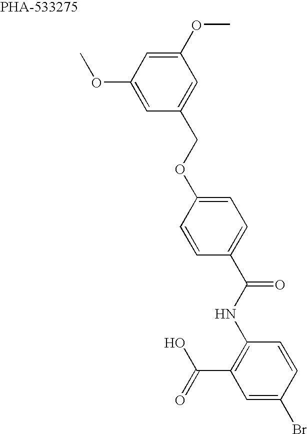

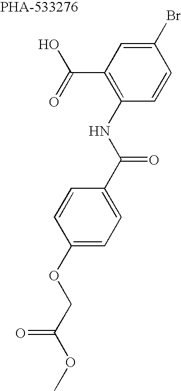

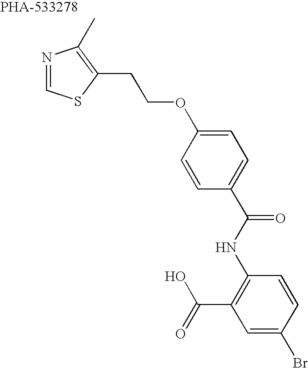

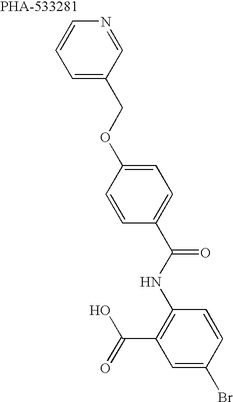

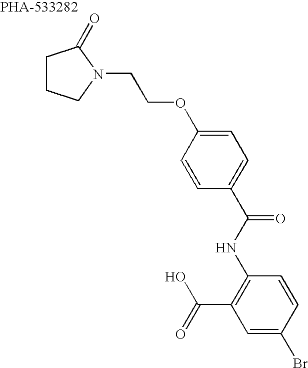

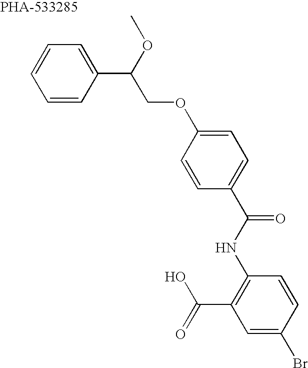

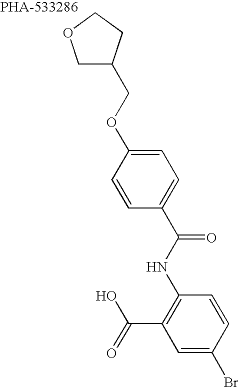

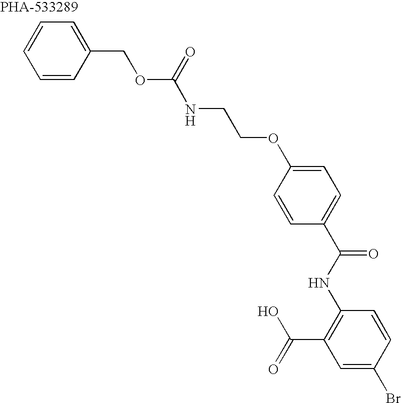

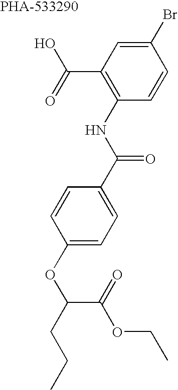

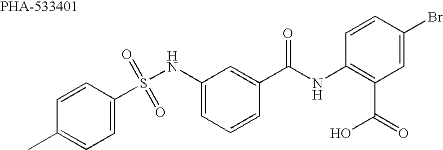

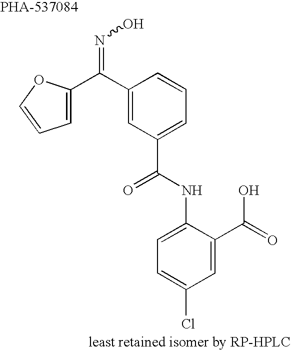

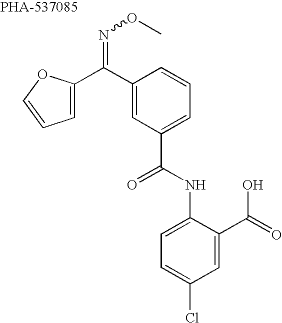

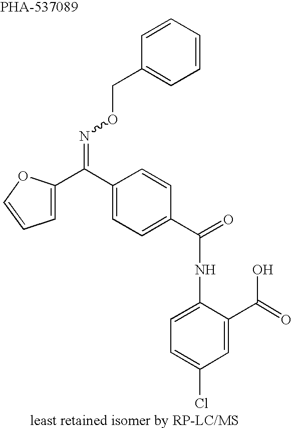

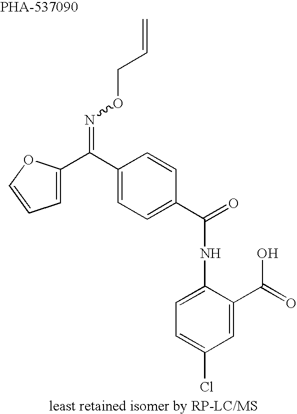

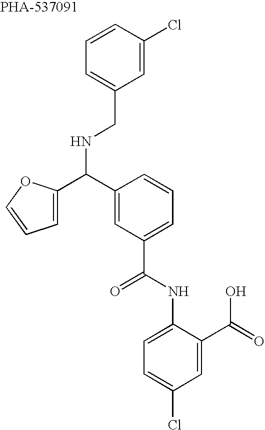

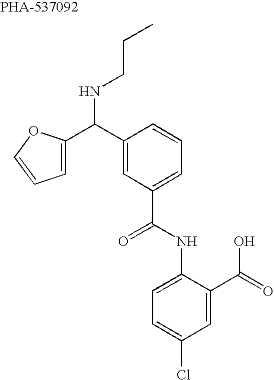

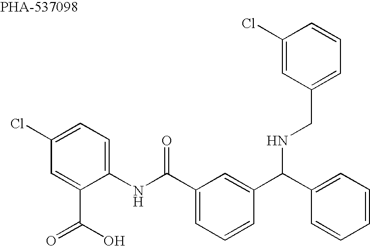

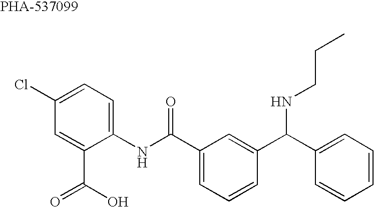

| Compound No., Structure | Compound No., Structure |
|---|---|
| 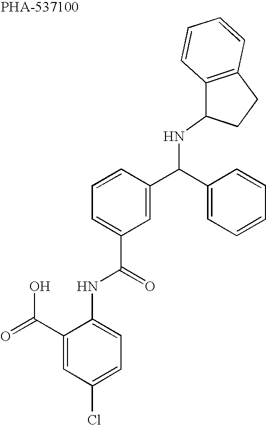 | 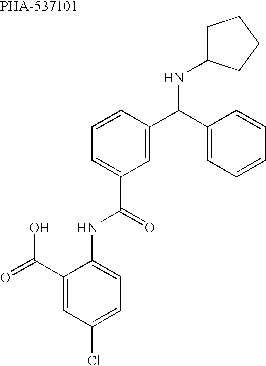 |
| 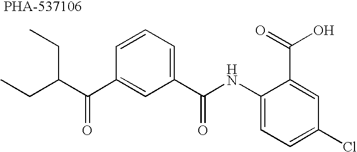 | 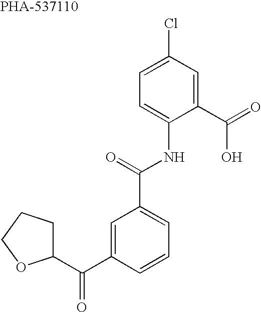 |
| 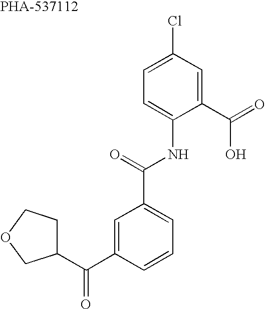 | 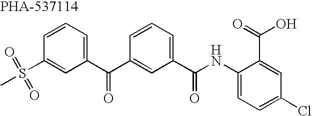 |
| 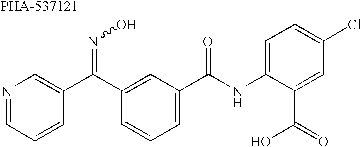 | 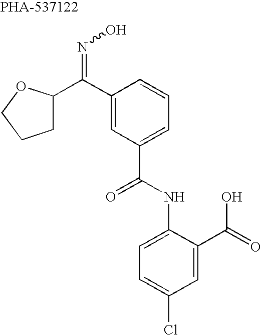 |
| 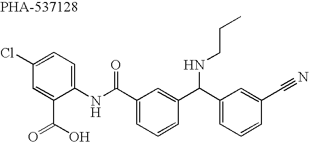 | 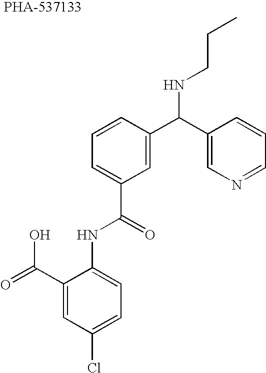 |
| 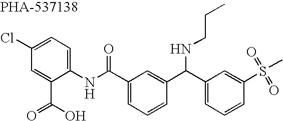 | 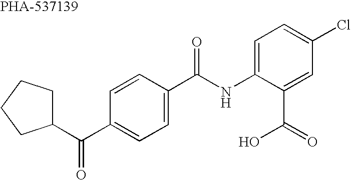 |
| 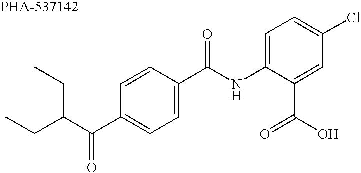 | 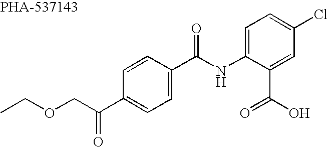 |
| 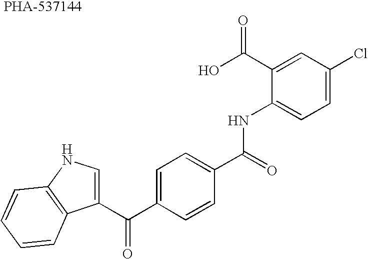 | 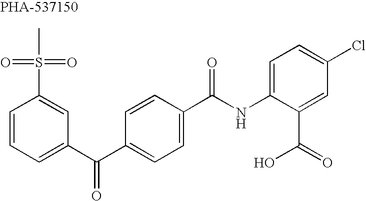 |
| 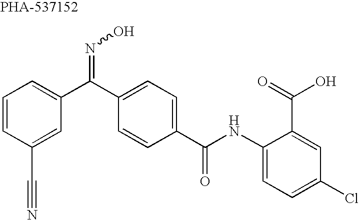 | 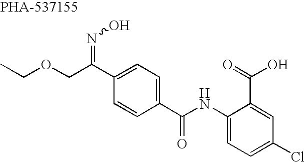 |
| 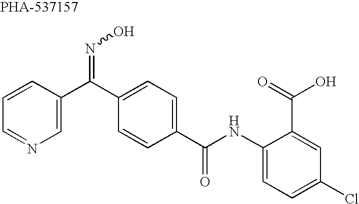 | 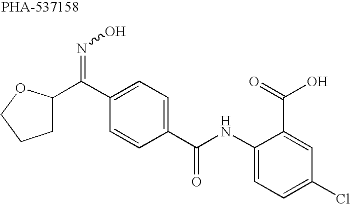 |
| 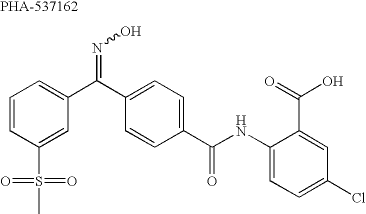 | 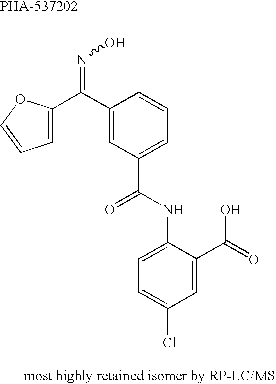 |
| 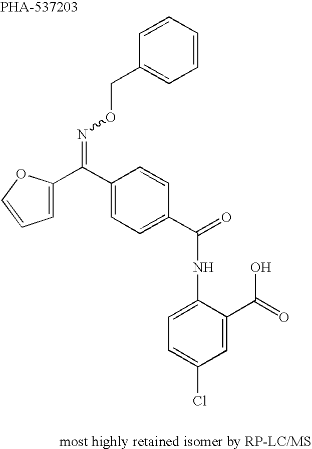 | 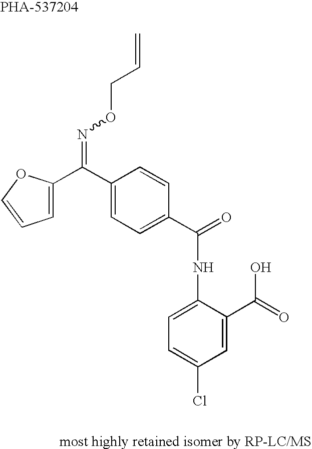 |
| 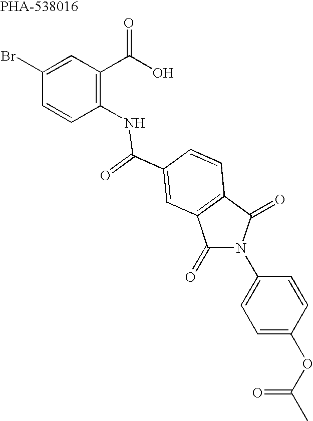 | 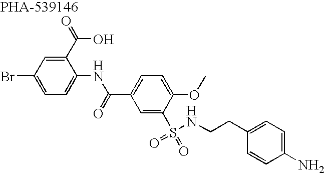 |
| 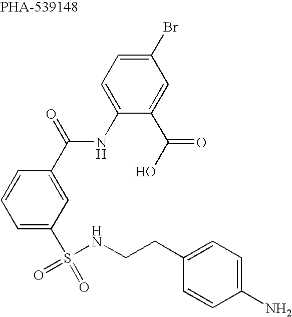 | 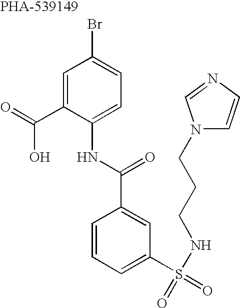 |
| 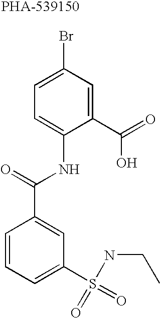 | 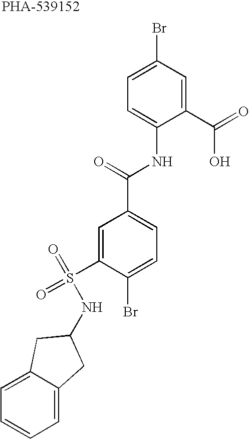 |
| 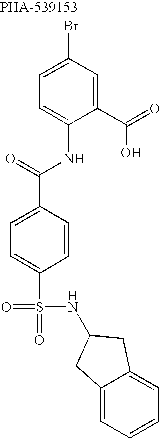 | 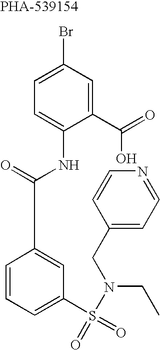 |
| 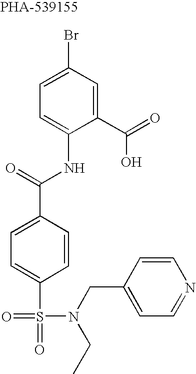 | 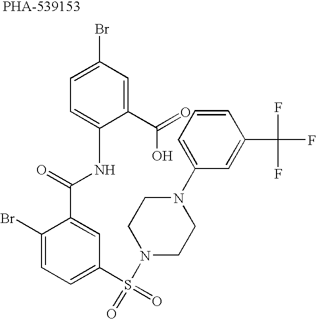 |
| 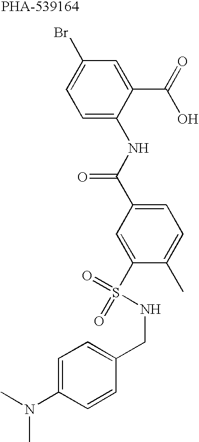 | 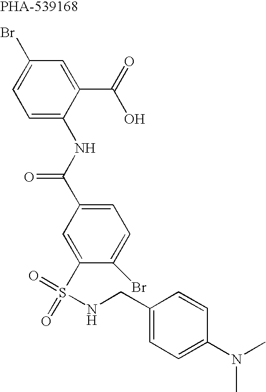 |
| 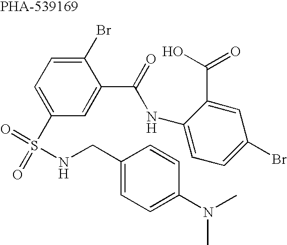 | 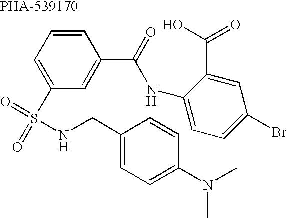 |
| 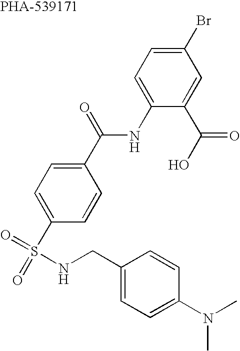 | 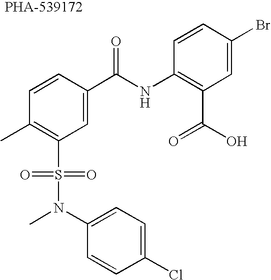 |
| 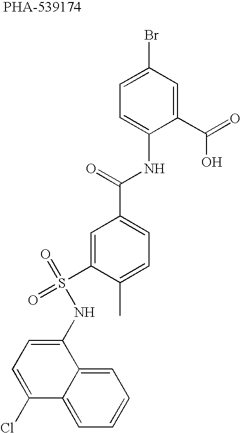 | 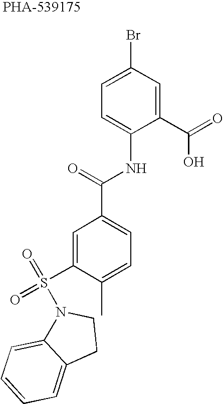 |
| 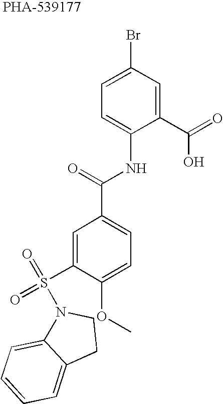 | 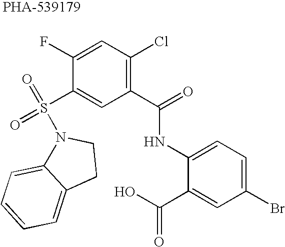 |
| 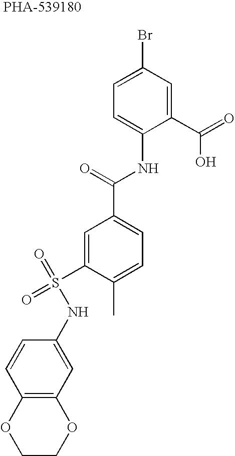 | 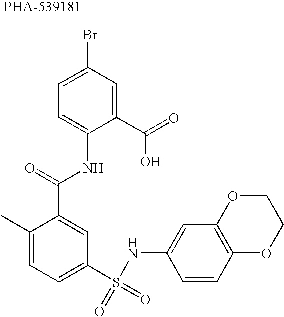 |
| 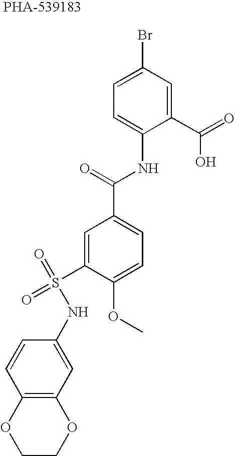 | 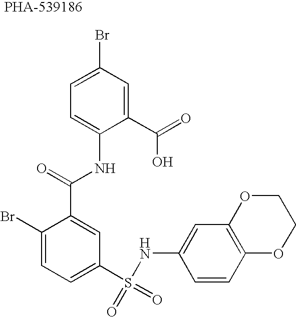 |
| 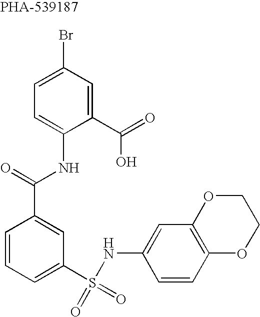 | 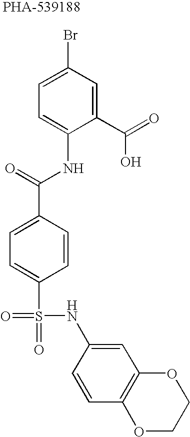 |
| 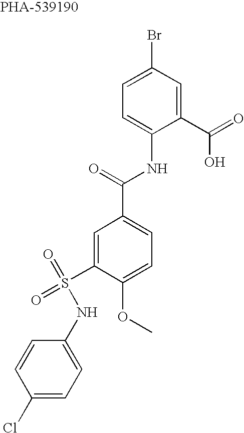 | 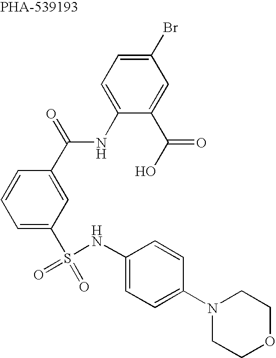 |
| 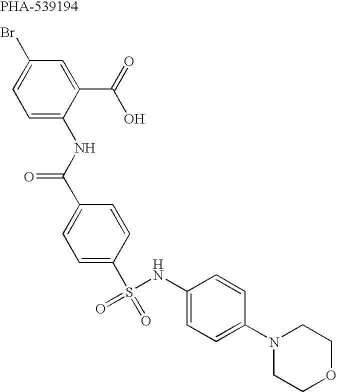 | 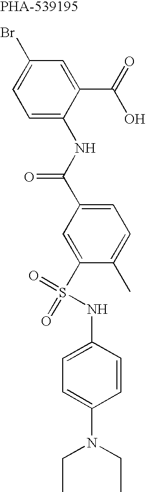 |
| 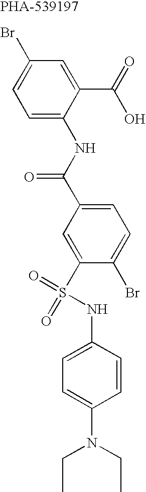 | 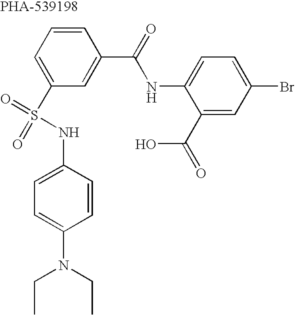 |
| 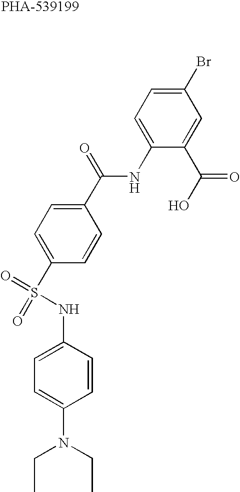 | 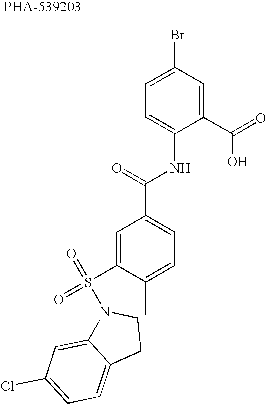 |
| 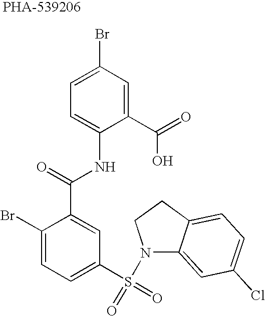 | 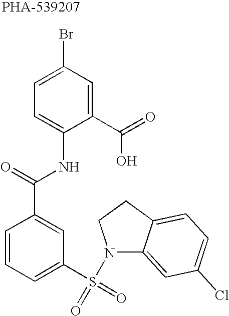 |
| 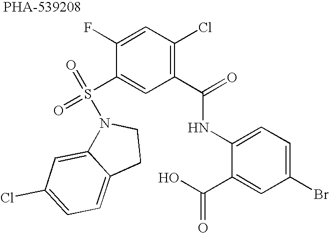 | 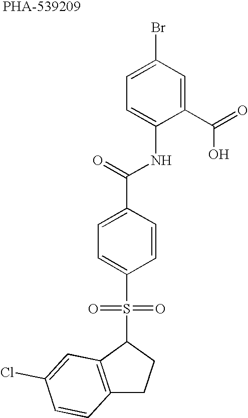 |
| 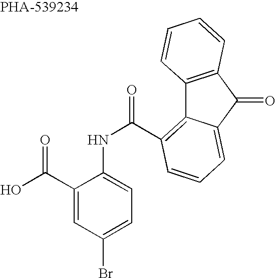 | 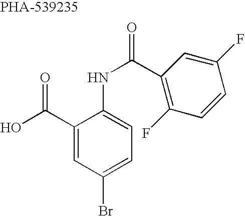 |
| 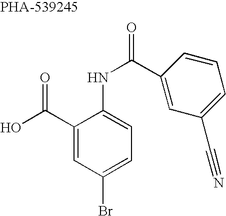 | 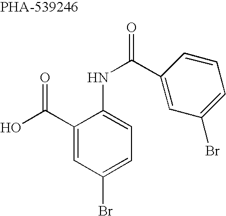 |
| 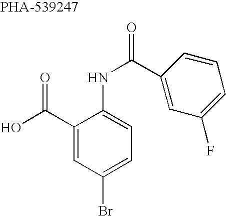 | 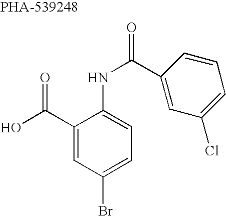 |
| 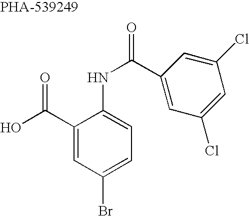 | 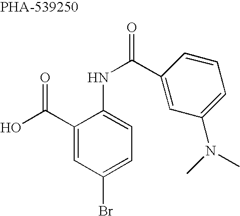 |
| 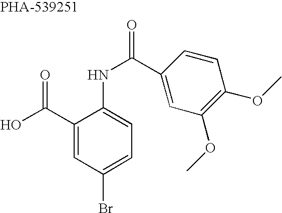 | 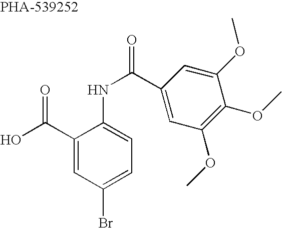 |
| 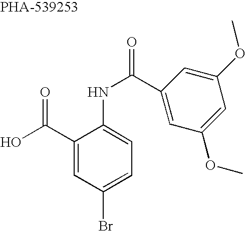 | 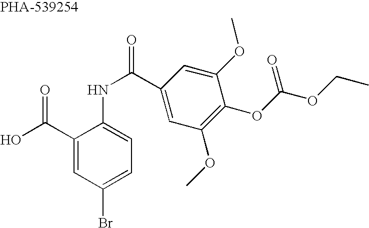 |
| 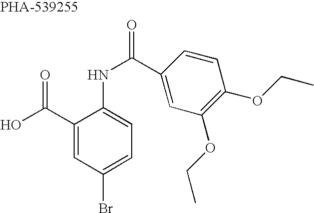 | 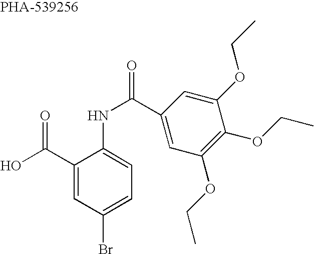 |
| 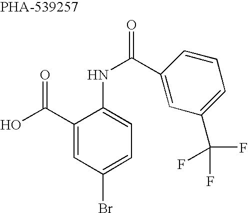 | 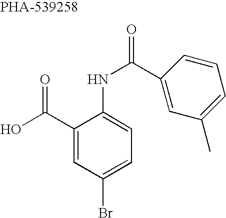 |
| 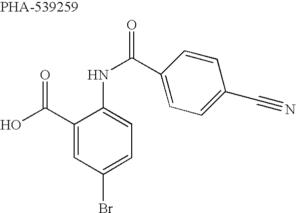 | 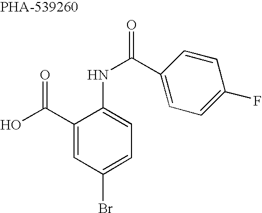 |
| 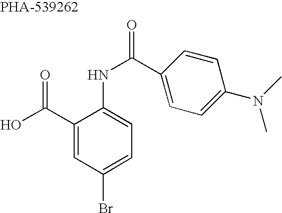 | 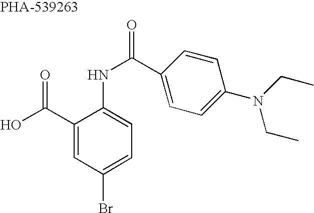 |
| 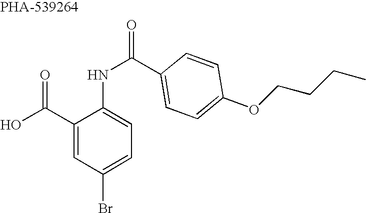 | 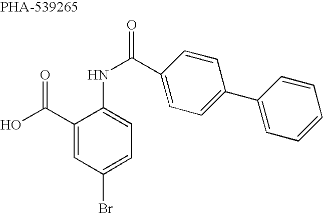 |
| 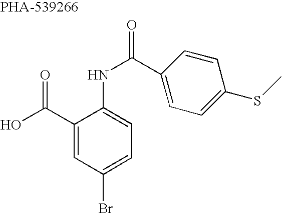 | 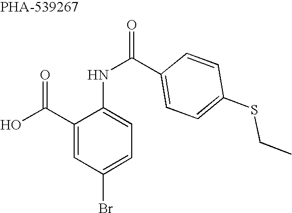 |
| 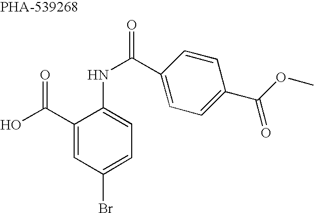 | 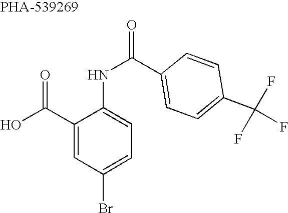 |
| 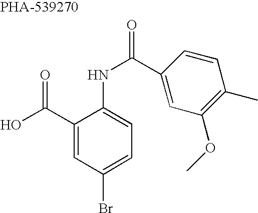 | 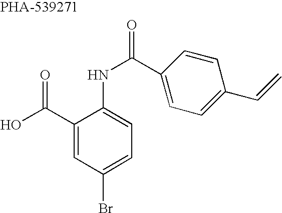 |
| 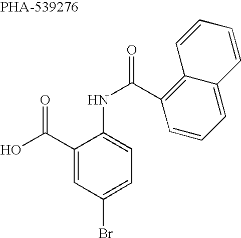 | 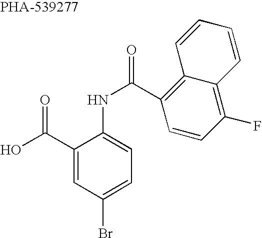 |
| 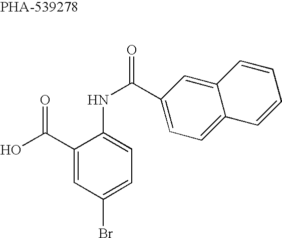 | 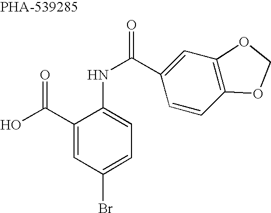 |
| 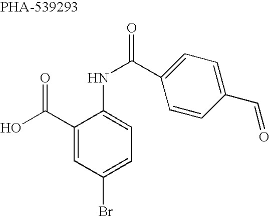 | 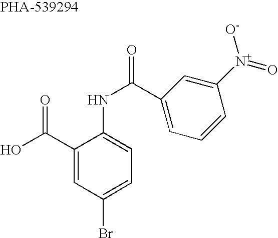 |
| 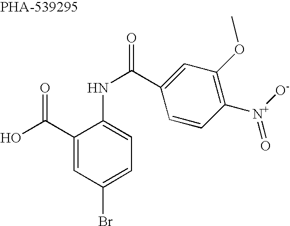 | 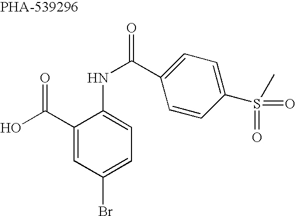 |
| 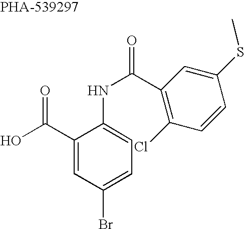 | 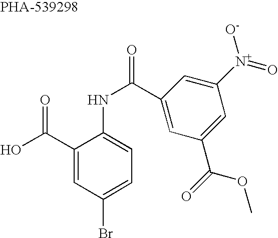 |
| 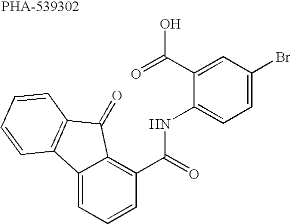 | 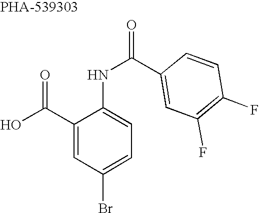 |
| 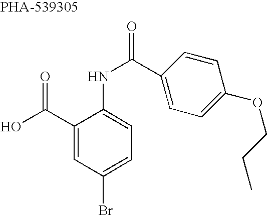 | 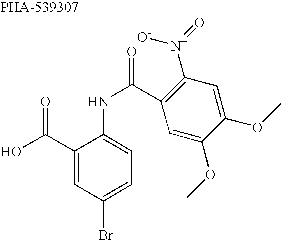 |
| 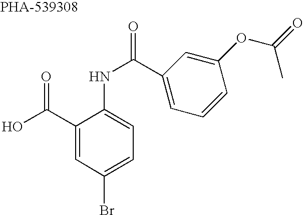 | 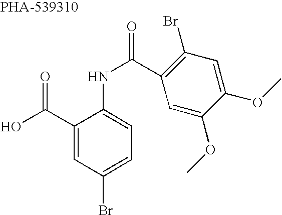 |
| 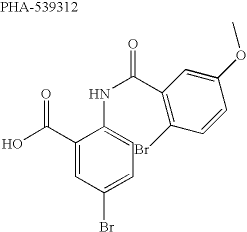 | 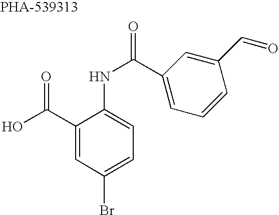 |
| 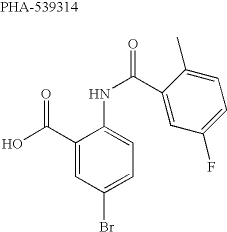 | 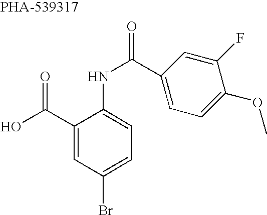 |
| 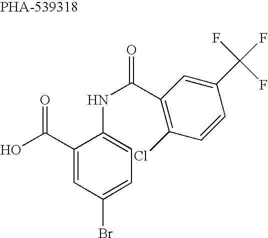 | 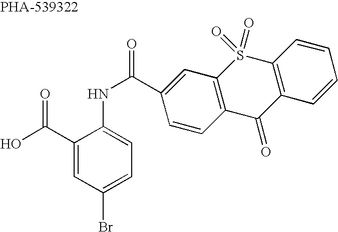 |
| 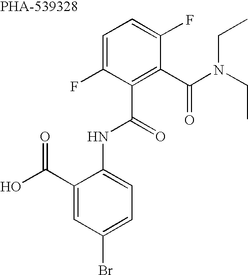 | 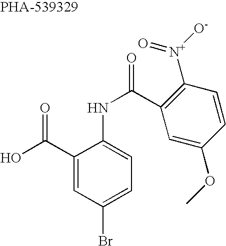 |
| 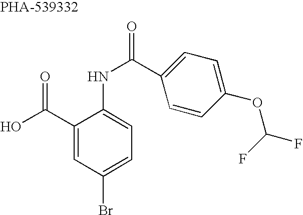 | 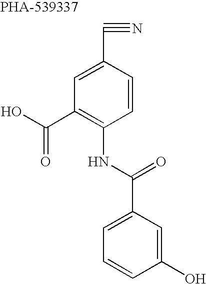 |
| 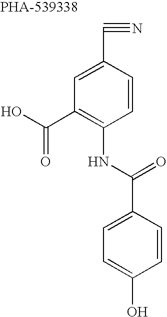 | 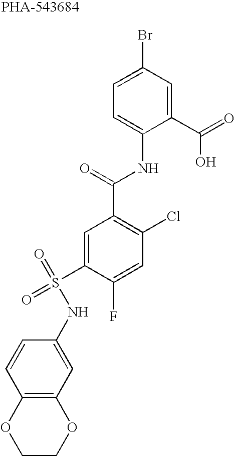 |
| 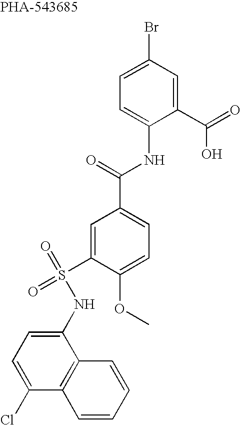 | 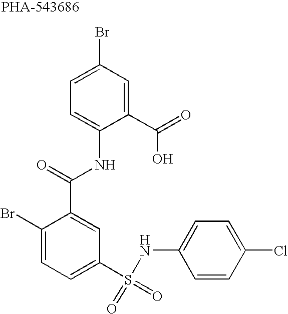 |
| 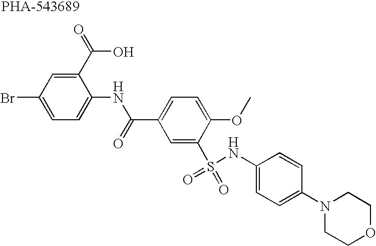 | 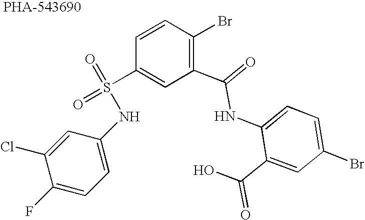 |
| 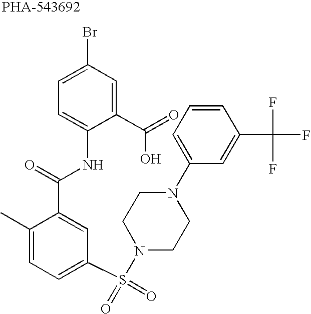 | 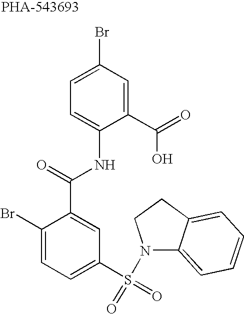 |
| 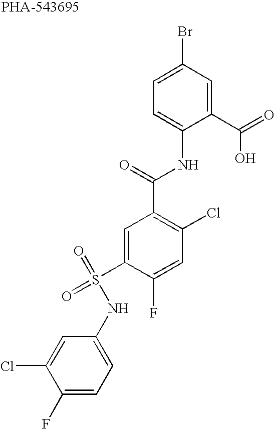 | 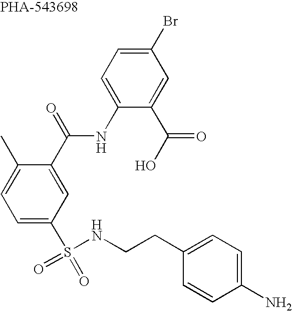 |
| 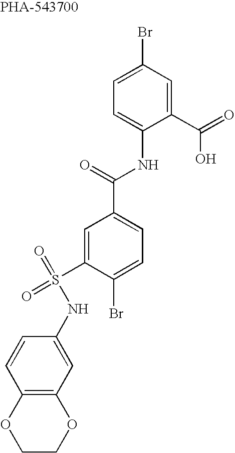 | 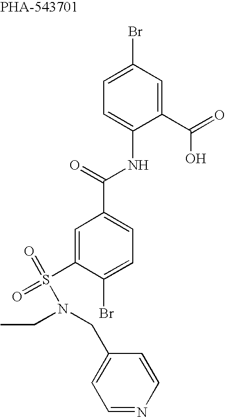 |
| 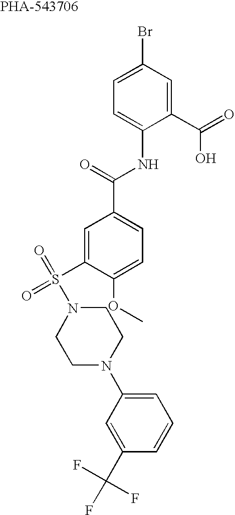 | 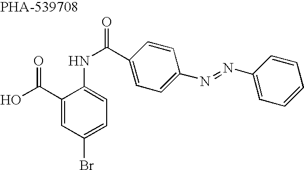 |
| 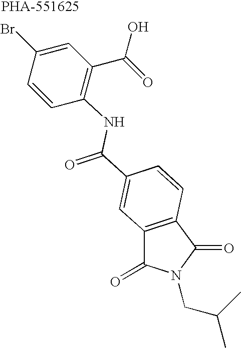 | 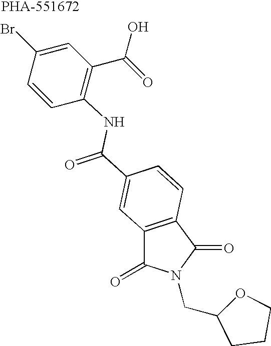 |
| 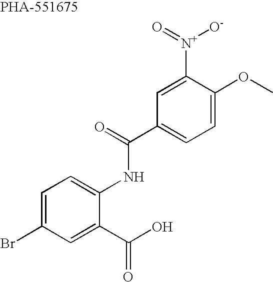 | 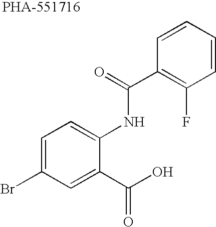 |
| 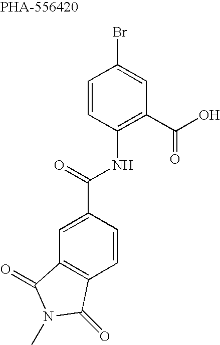 | 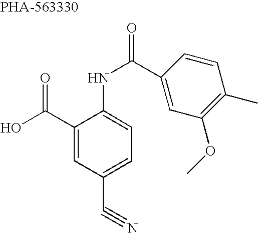 |
| 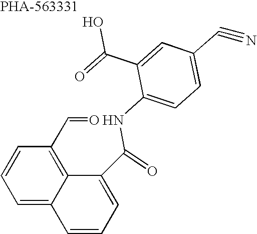 | 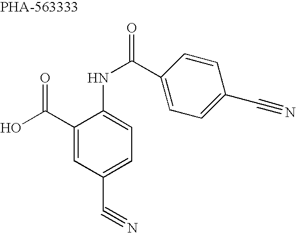 |
| 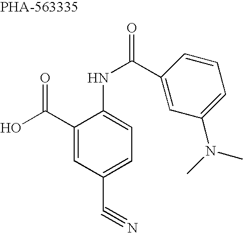 | 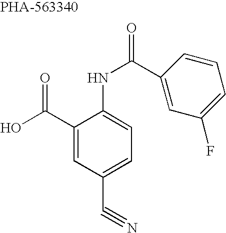 |
| 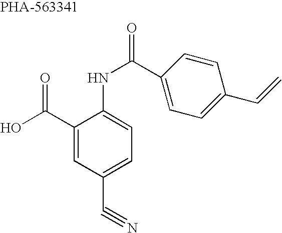 | 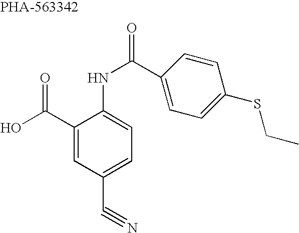 |
| 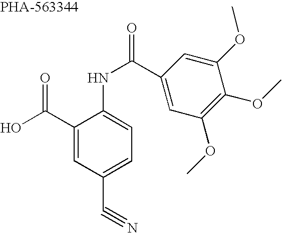 | 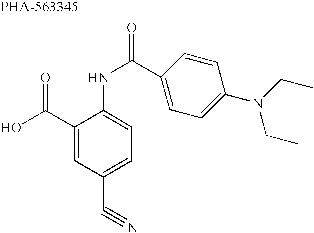 |
| 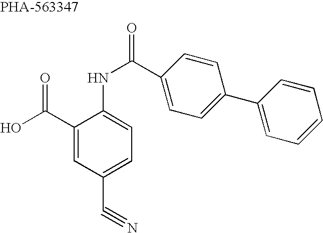 | 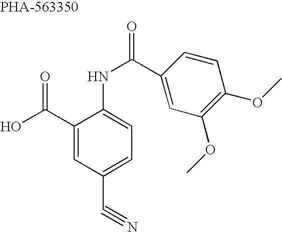 |
| 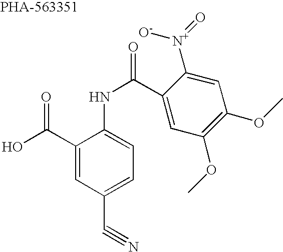 | 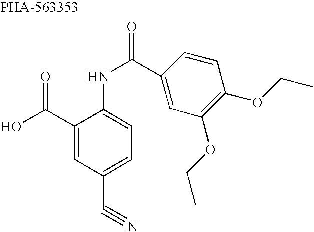 |
| 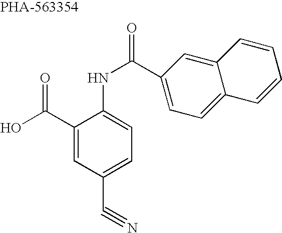 | 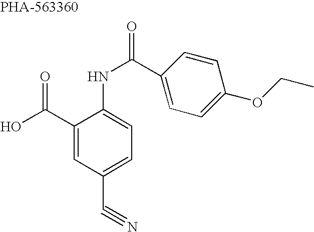 |

| Compound No., Structure | Compound No., Structure |
|---|---|
| 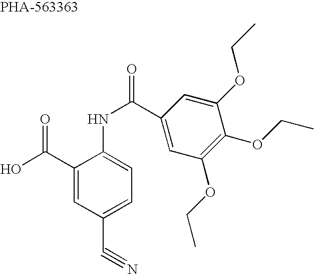 | 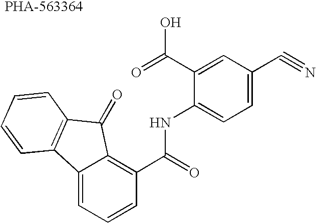 |
| 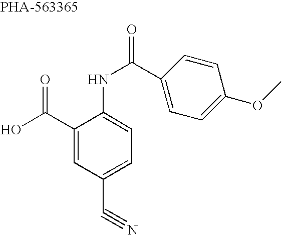 | 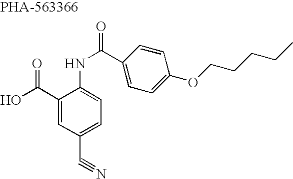 |
| 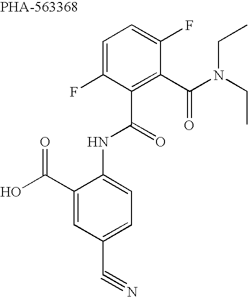 | 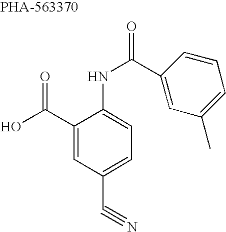 |
| 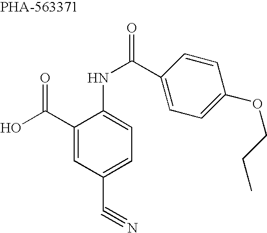 | 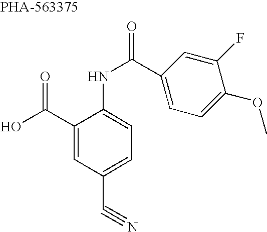 |
| 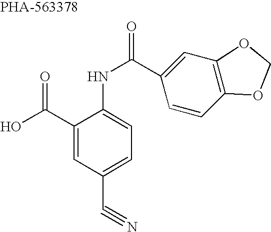 | 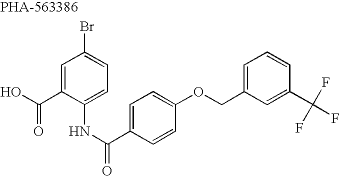 |
| 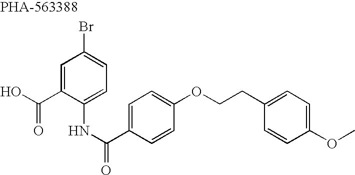 | 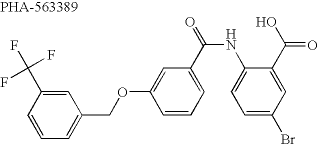 |
| 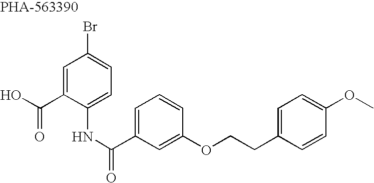 | 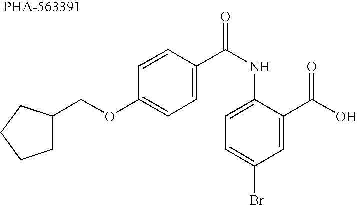 |
| 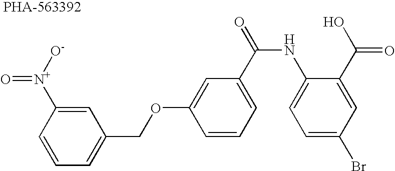 | 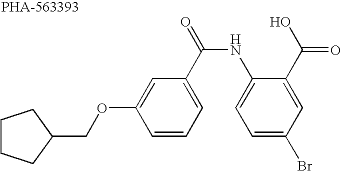 |
| 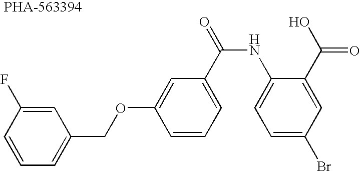 | 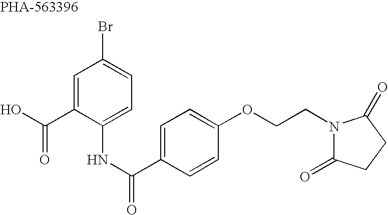 |
| 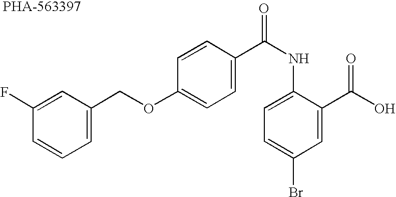 | 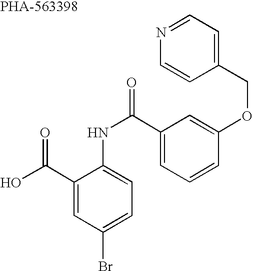 |
| 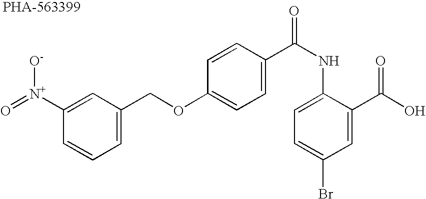 | 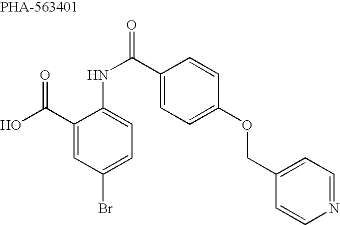 |
| 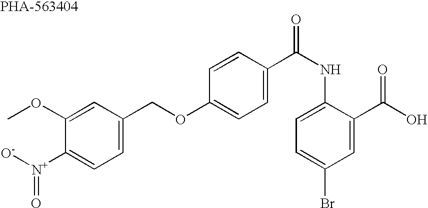 | 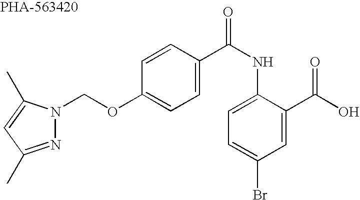 |
| 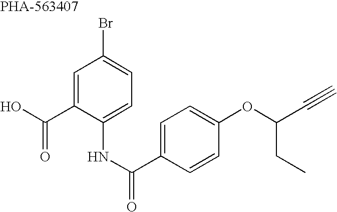 | 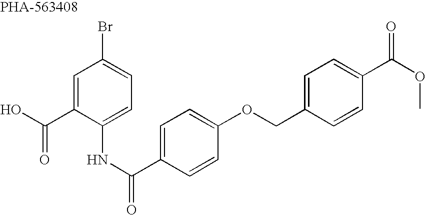 |
| 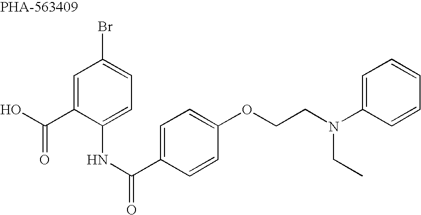 | 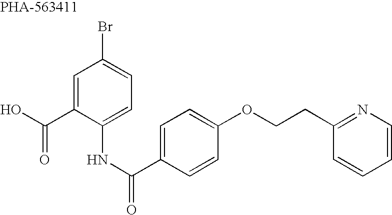 |
| 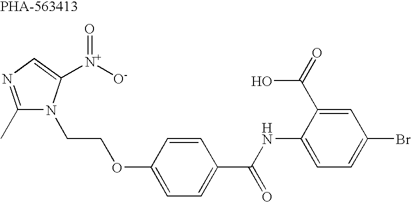 | 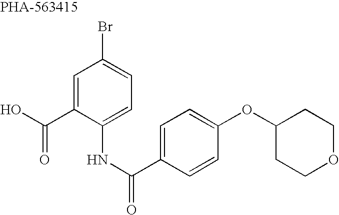 |
| 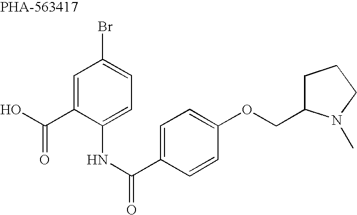 | 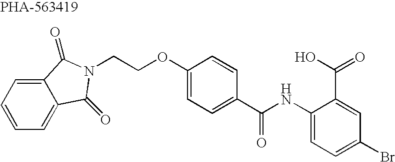 |
| 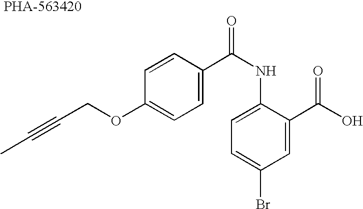 | 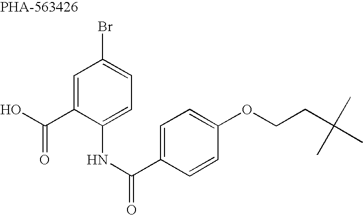 |
| 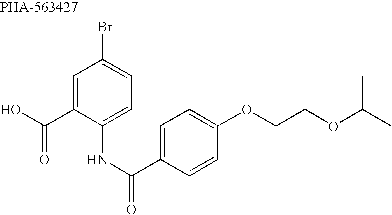 | 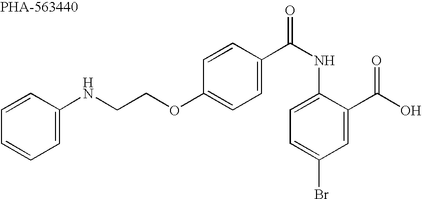 |
| 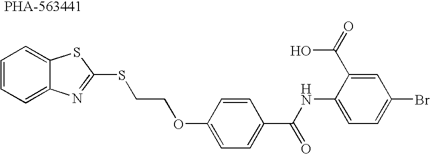 | 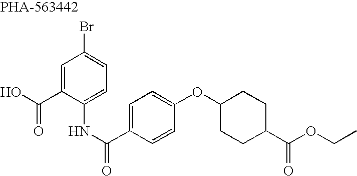 |
| 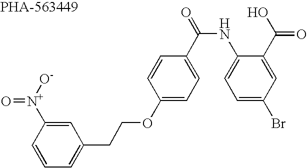 | 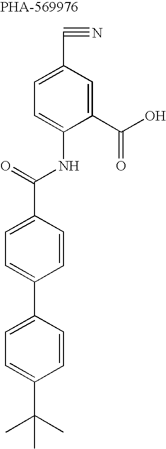 |
| 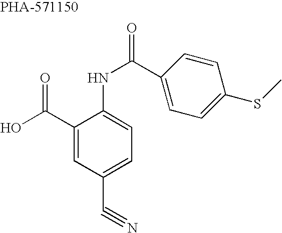 | 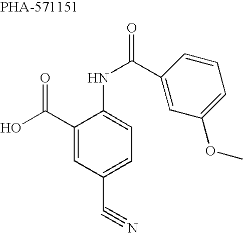 |
| 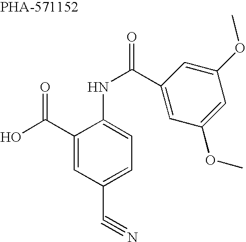 | 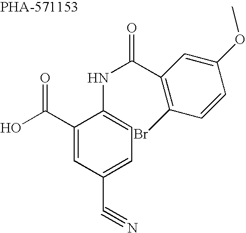 |
| 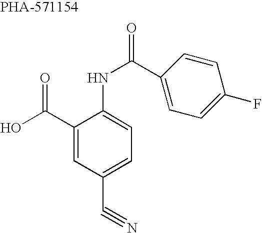 | 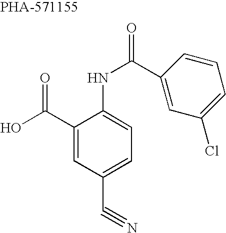 |
| 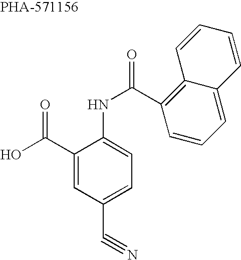 | 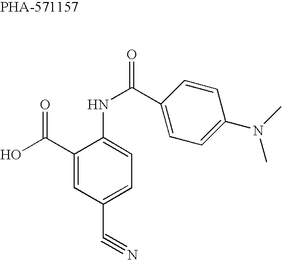 |
| 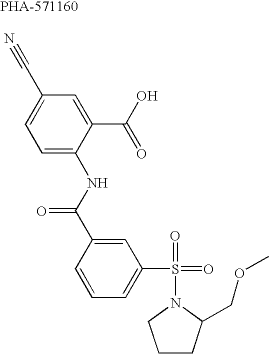 | 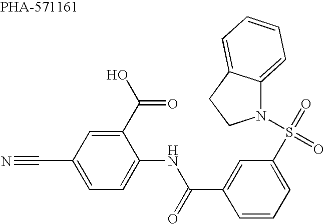 |
| 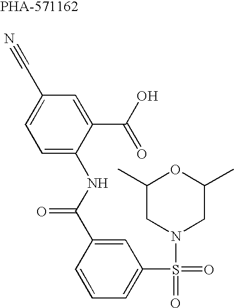 | 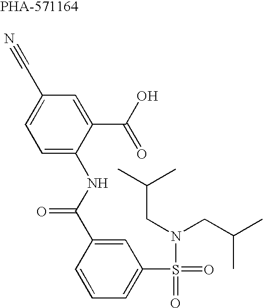 |
| 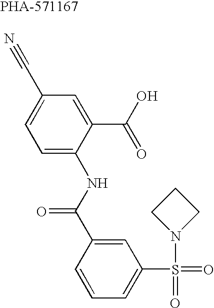 | 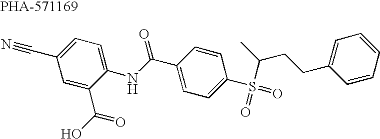 |
| 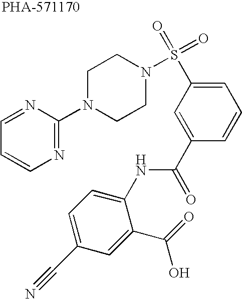 | 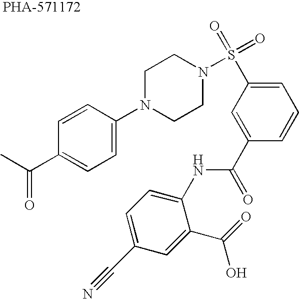 |
| 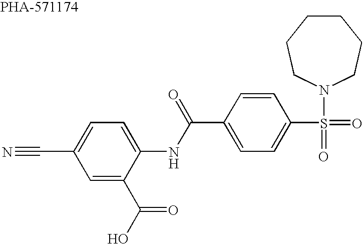 | 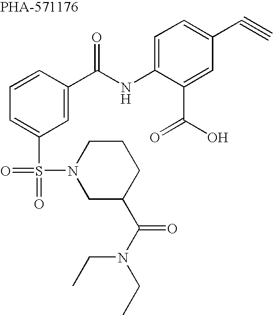 |
| 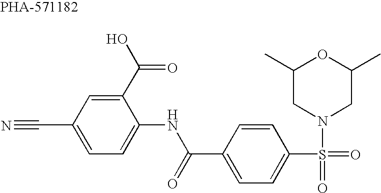 | 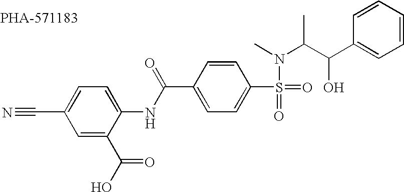 |
| 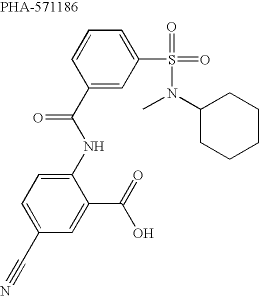 | 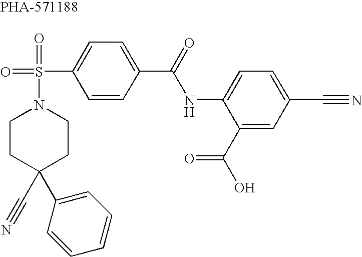 |
| 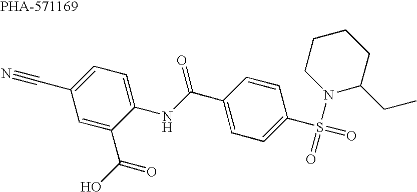 | 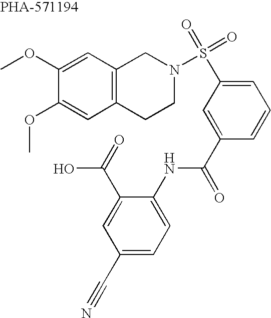 |
| 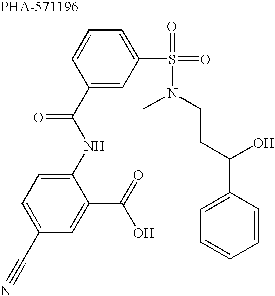 | 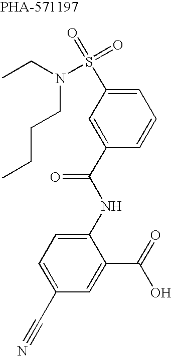 |
| 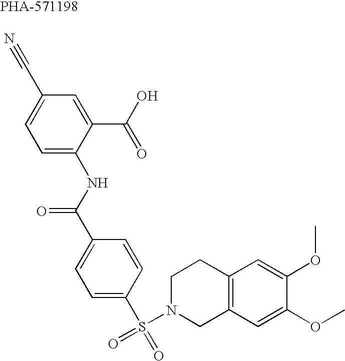 | 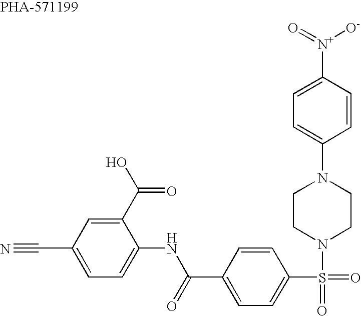 |
| 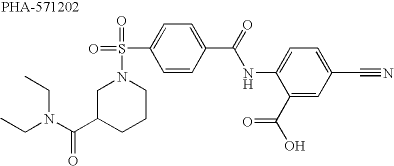 | 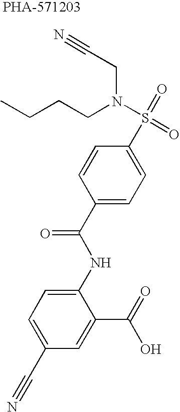 |
| 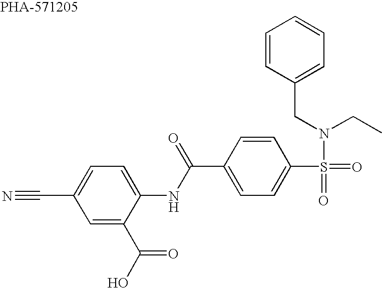 | 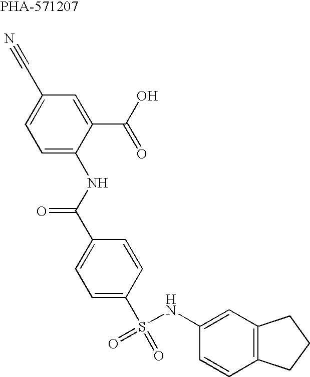 |
| 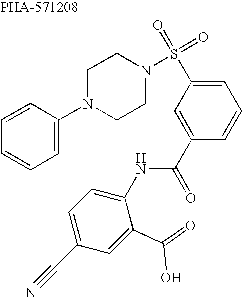 | 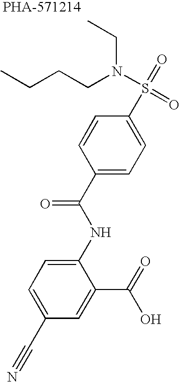 |
| 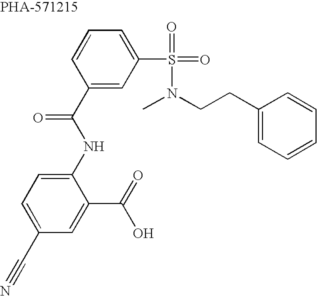 | 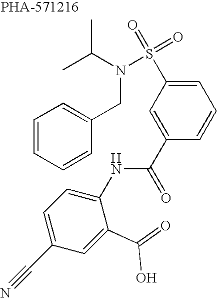 |
| 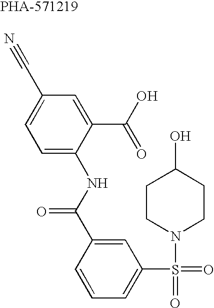 | 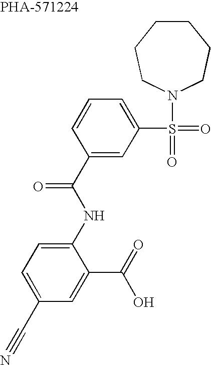 |
| 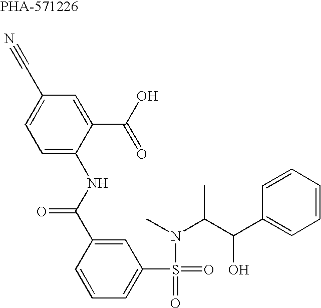 | 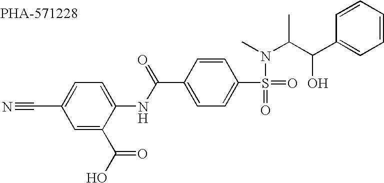 |
| 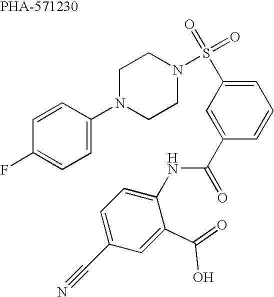 | 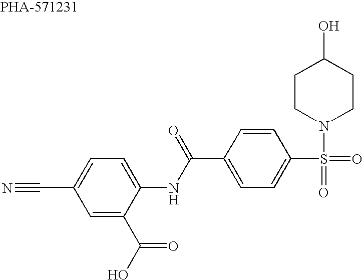 |
| 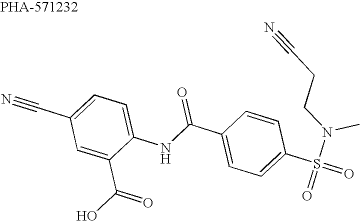 | 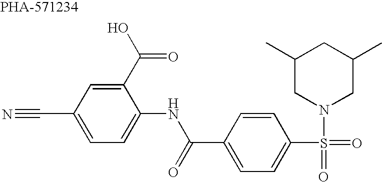 |
| 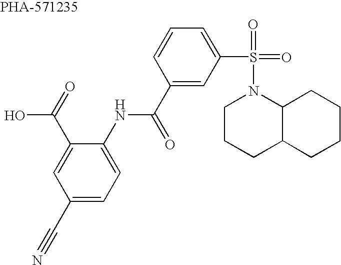 | 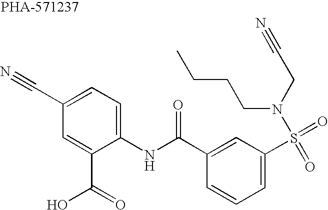 |
| 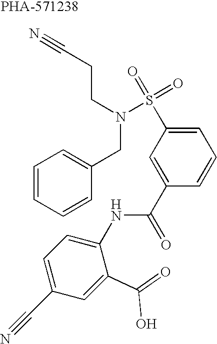 | 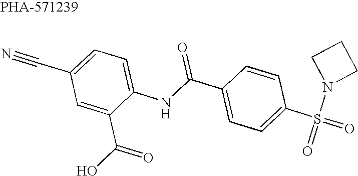 |
| 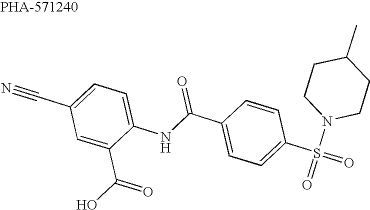 | 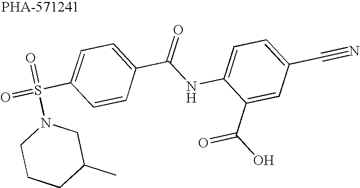 |
| 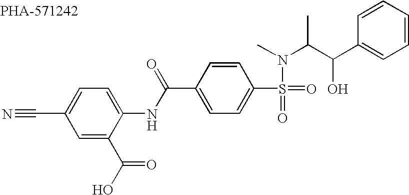 | 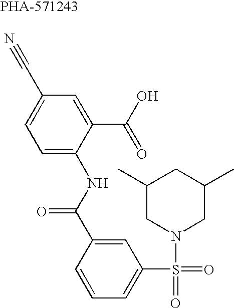 |
| 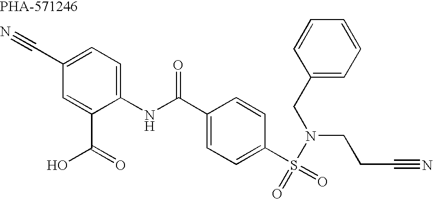 | 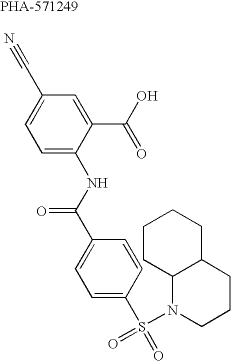 |
| 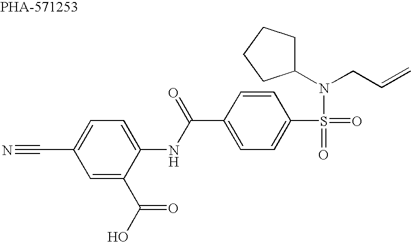 | 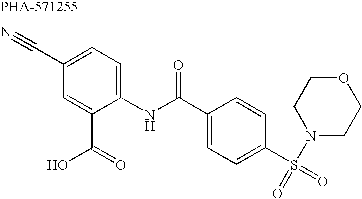 |
| 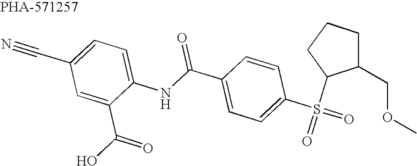 | 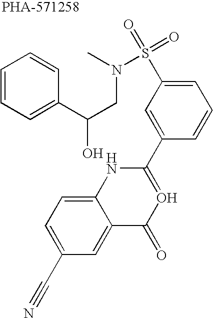 |
| 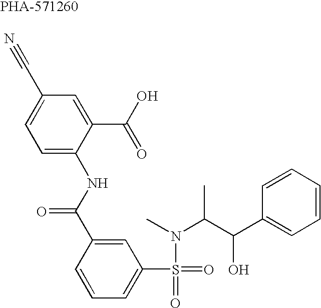 | 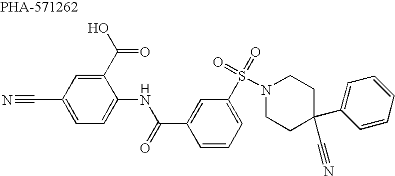 |
| 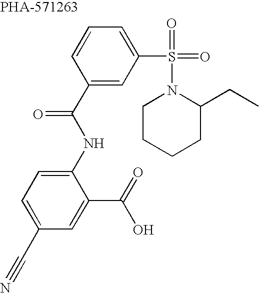 | 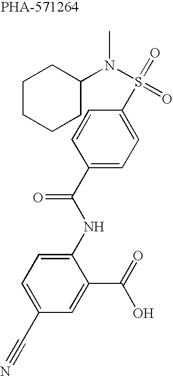 |
| 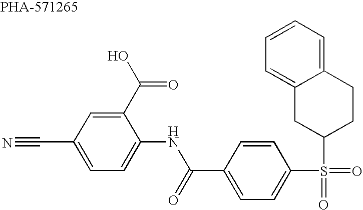 | 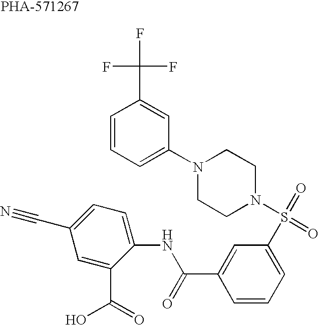 |
| 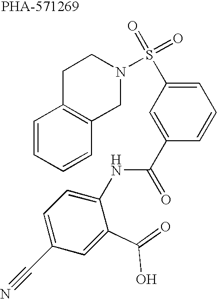 | 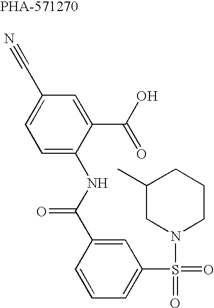 |
| 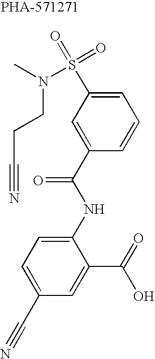 | 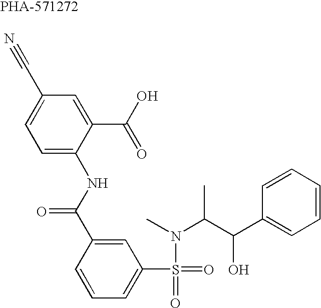 |
| 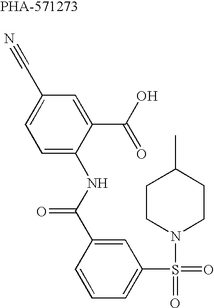 | 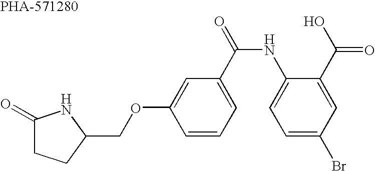 |
| 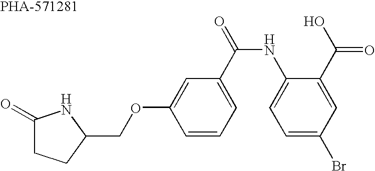 | 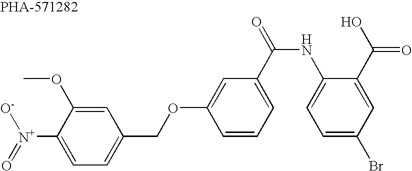 |
| 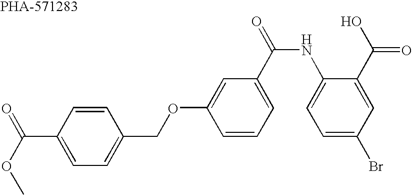 | 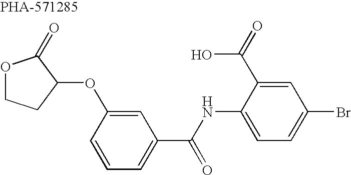 |
| 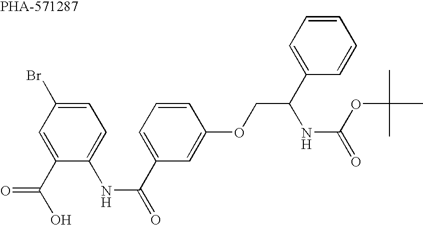 | 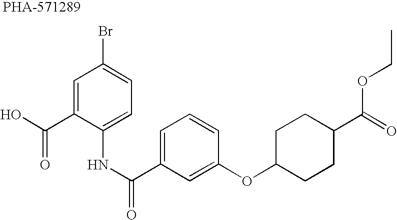 |
| 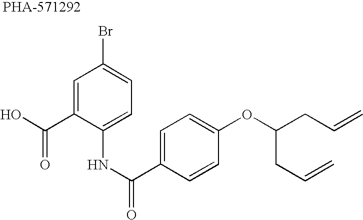 | 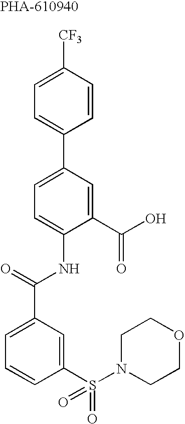 |
| 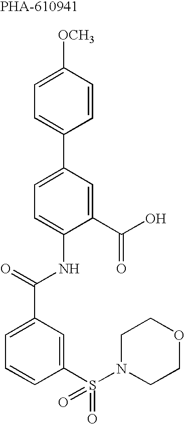 | 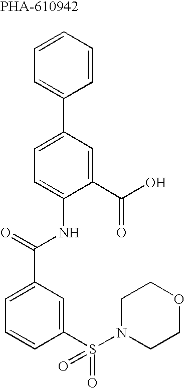 |
| 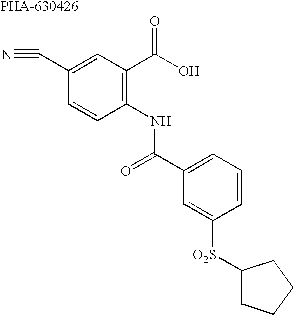 | 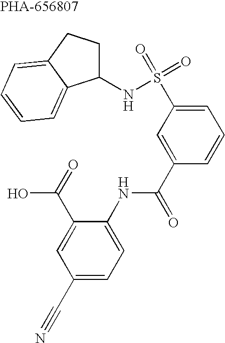 |
| 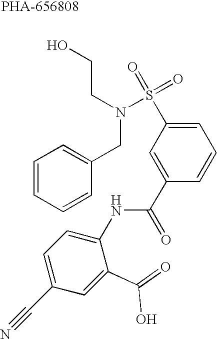 | 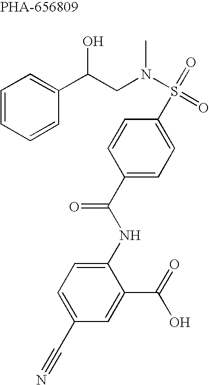 |
| 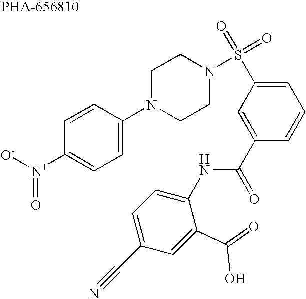 | 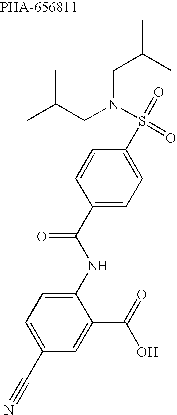 |
| 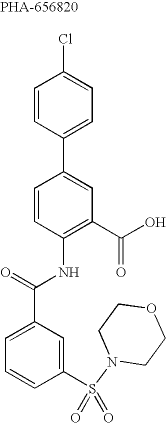 | 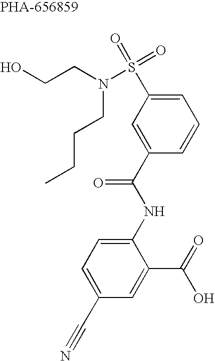 |
| 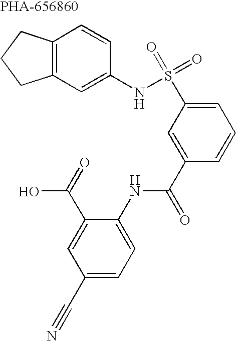 | 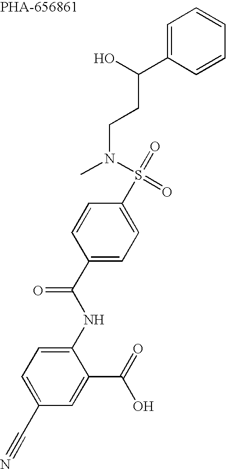 |
| 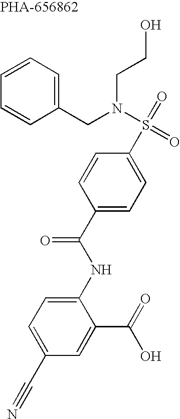 | 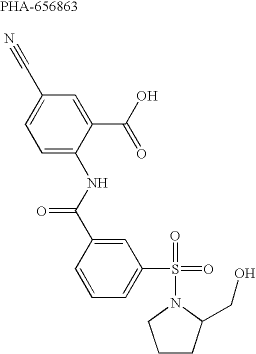 |
| 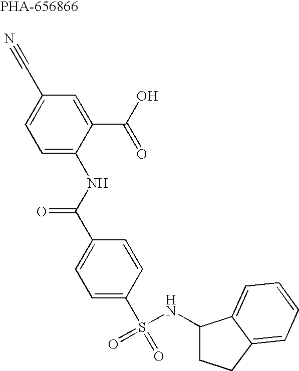 | 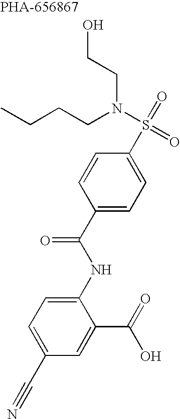 |
| 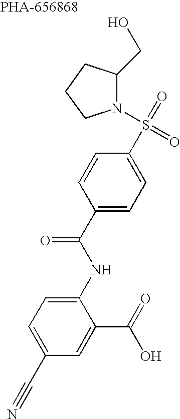 | 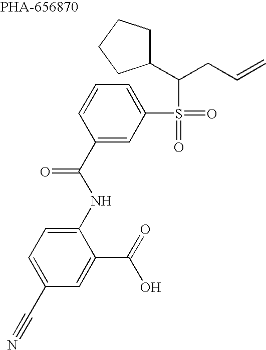 |
| 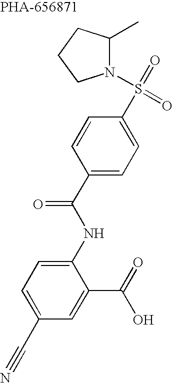 | 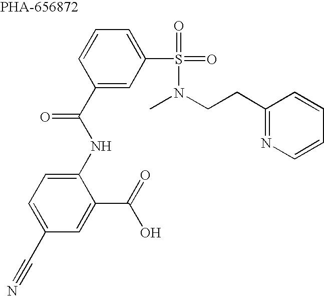 |
| 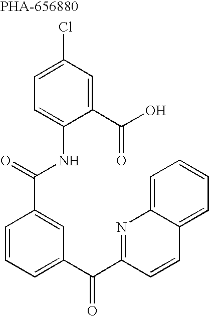 | 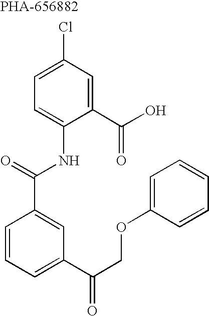 |
| 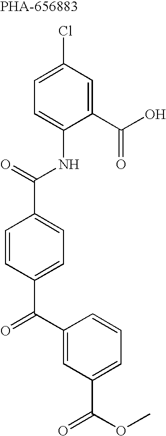 | 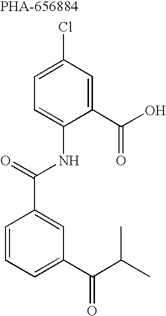 |
| 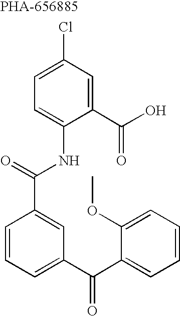 | 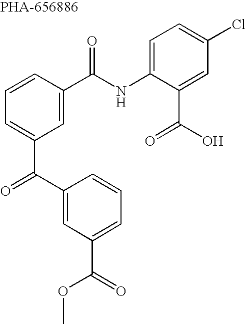 |
| 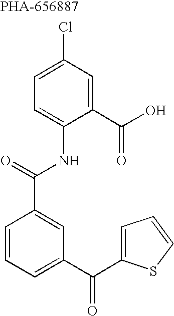 | 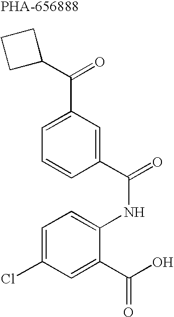 |
| 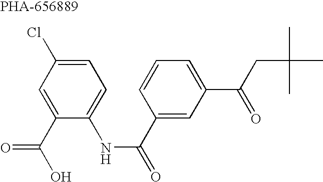 | 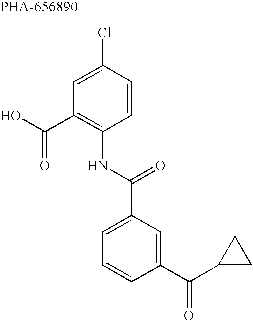 |
| 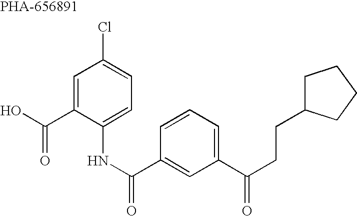 | 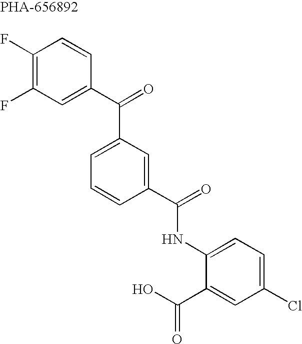 |
| 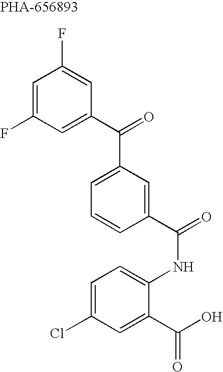 | 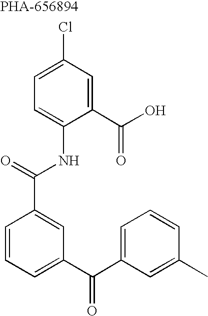 |
| 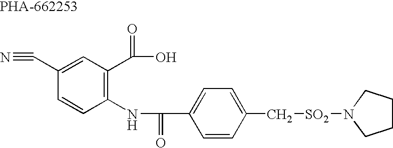 | 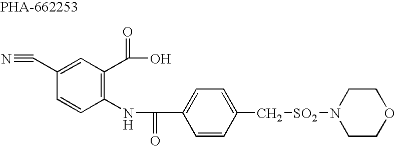 |
| 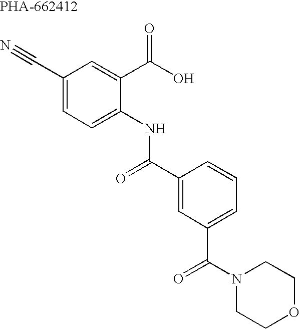 | 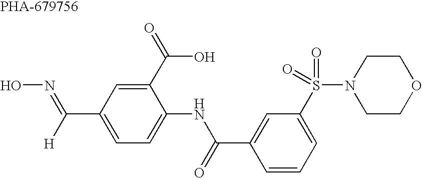 |
| 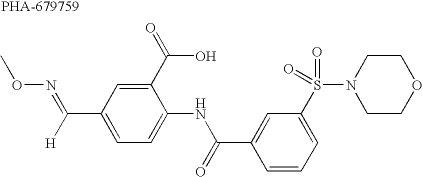 | 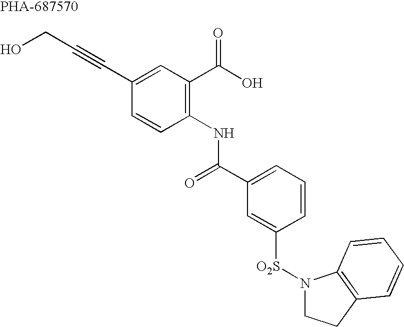 |
| 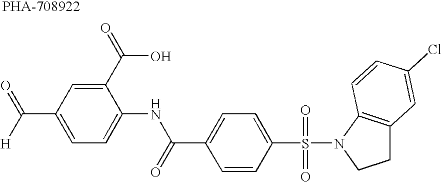 | 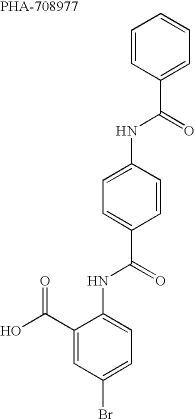 |
| 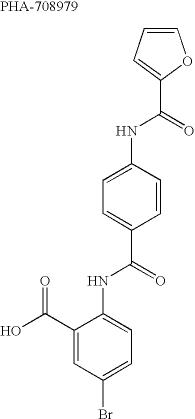 | 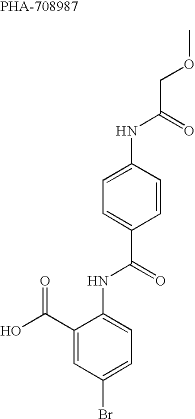 |
| 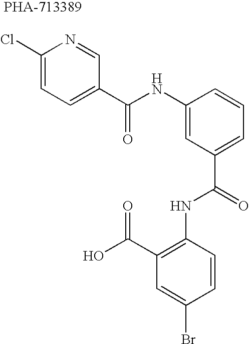 | 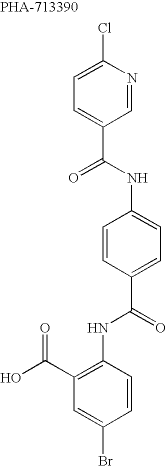 |
| 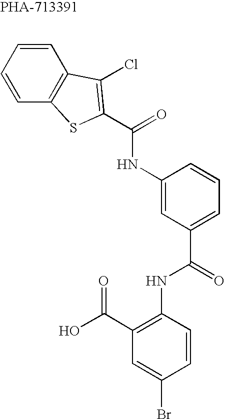 | 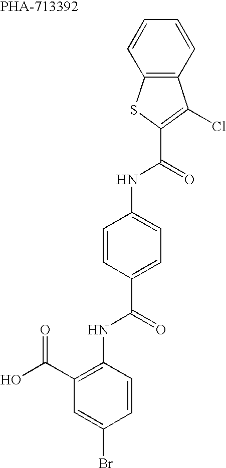 |
| 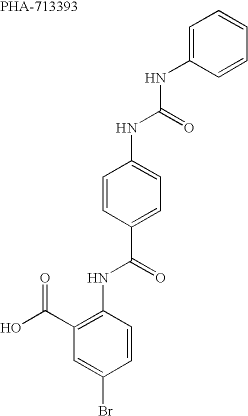 | 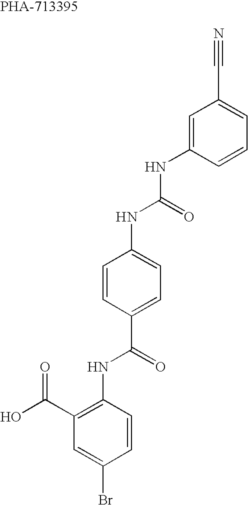 |
| 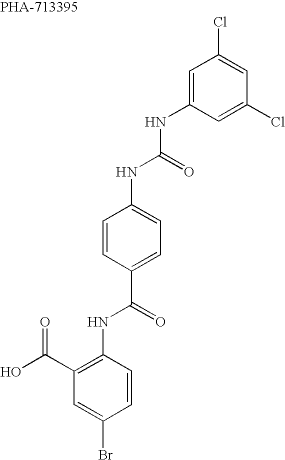 | 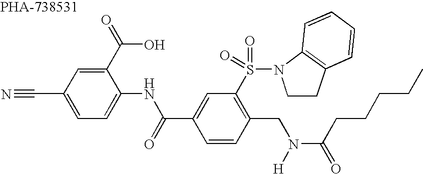 |
| 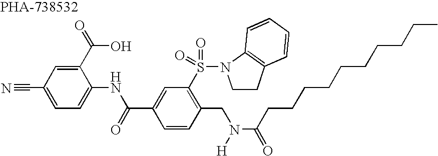 | 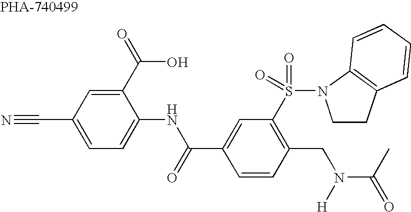 |
| 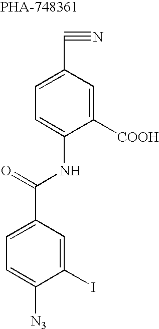 | 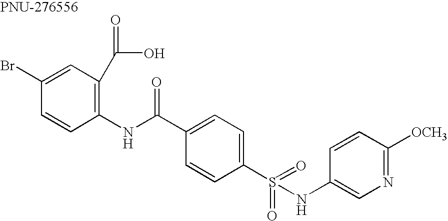 |
| 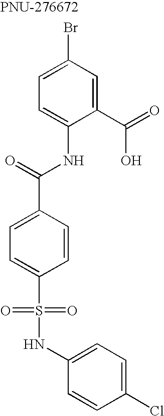 | 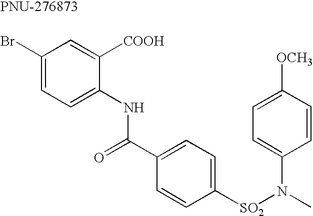 |
| 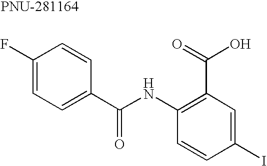 | 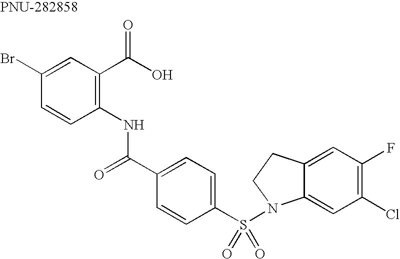 |
| 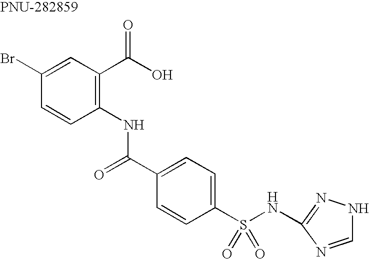 | 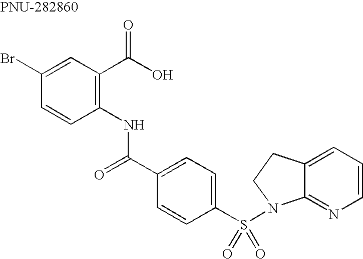 |
| 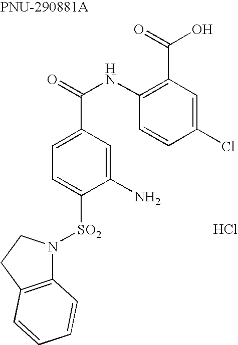 | 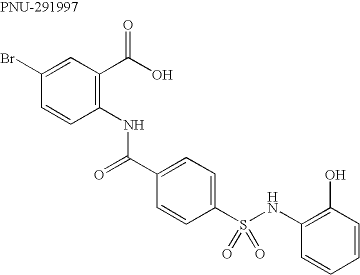 |
| 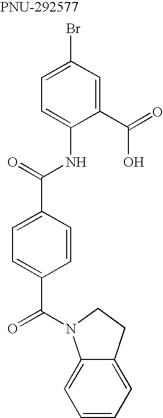 | |

### Example 11

### Activity Data

MIC Test Method

The in vitro MICs of test compounds were determined by a standard agar dilution method. A stock drug solution of each analog was prepared in the preferred solvent, usually DMSO:H₂O (1:3). Serial 2-fold dilutions of each sample are made using 1.0 ml aliquots of sterile distilled water. To each 1.0 ml aliquot of drug was added 9 ml of molten Mueller Hinton agar medium. The drug-supplemented agar was mixed, poured into 15×100 mm petri dishes, and allowed to solidify and dry prior to inoculation.

Vials of each of the test organisms are maintained frozen in the vapor phase of a liquid nitrogen freezer. Test cultures are grown overnight at 35° C. on the medium appropriate for the organism. Colonies are harvested with a sterile swab, and cell suspensions are prepared in Trypticase Soy broth (TSB) to equal the turbidity of a 0.5 McFarland standard. A 1:20 dilution of each suspension was made in TSB. The plates containing the drug supplemented agar are inoculated with a 0.001 ml drop of the cell suspension using a Steers replicator, yielding approximately 10⁴ to 10⁵ cells per spot. The plates are incubated overnight at 35° C.

Following incubation the Minimum Inhibitory Concentration (MIC μg/ml), the lowest concentration of drug that inhibits visible growth of the organism, was read and recorded. The data is shown in Tables I and II.

TABLE 1
Activity Data

| | SA 9218 |
|---|---|
| Compound No., Structure | MIC |
| L-217792 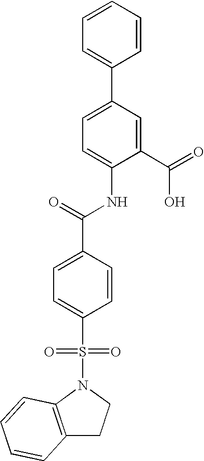 | 8 |
| PHA-501684 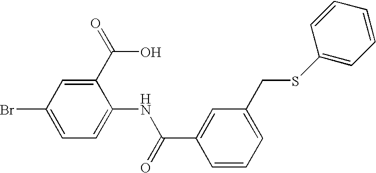 | 1 |
| PHA-501685 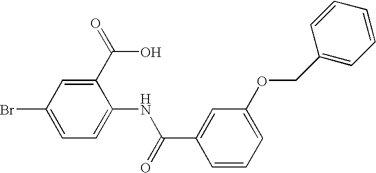 | 2 |
| PHA-501748 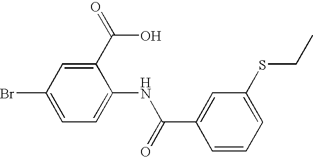 | 2 |
| PHA-504639 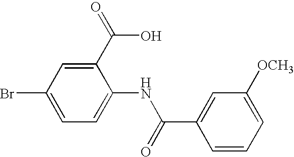 | 4 |
| PHA-515448 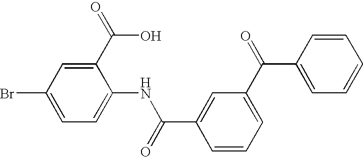 | 2 |
| PHA-515585 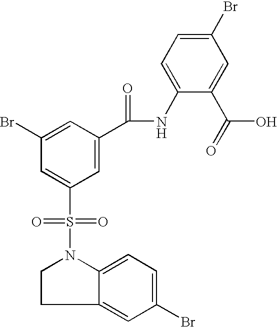 | 1 |
| PHA-516113 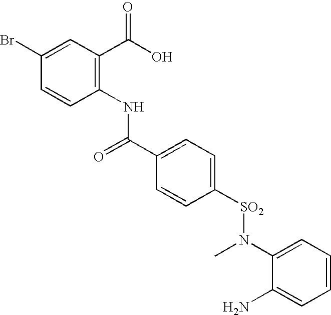 | 2 |
| PHA-519402 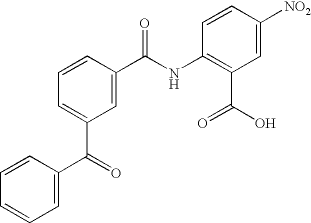 | 0.5 |
| PHA-521534 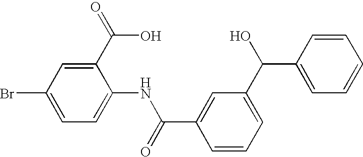 | 1 |
| PHA-522145 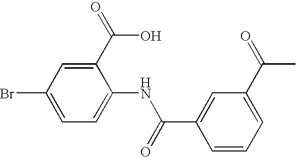 | 32 |
| PHA-524523 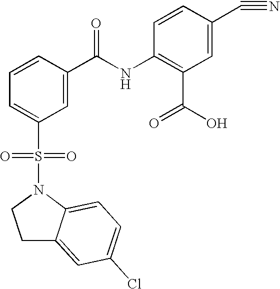 | 0.125 |
| PHA-524545 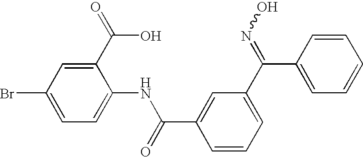 | 0.25 |
| PHA-526580 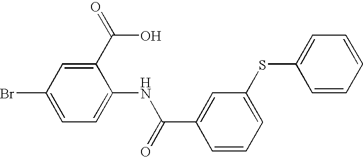 | 1 |
| PHA-530687 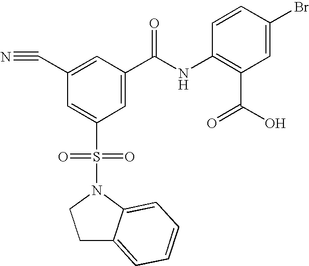 | 8 |
| PHA-535548 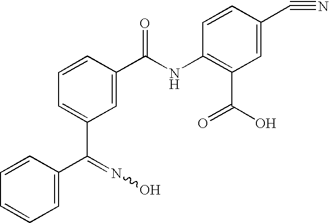 | 0.25 |
| PHA-535549 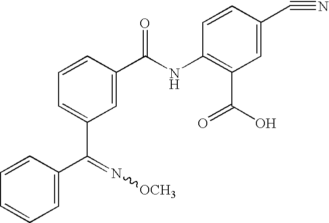 | 0.25 |
| PHA-535553 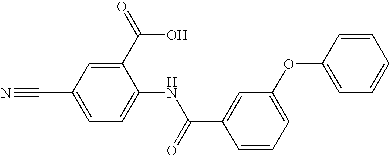 | 1 |
| PHA-543140 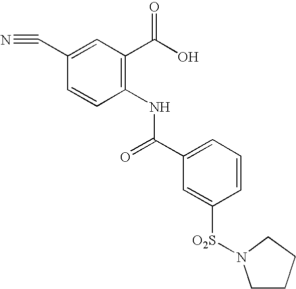 | 1 |
| PHA-546926 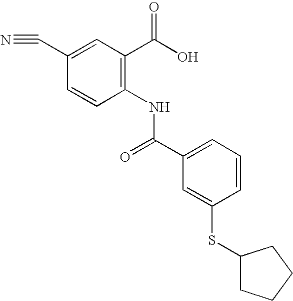 | 0.5 |
| PHA-547267 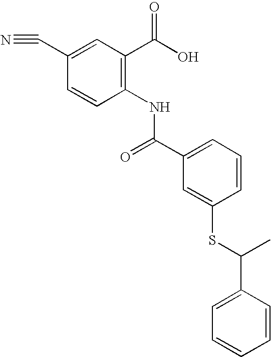 | 0.125 |
| PHA-552831 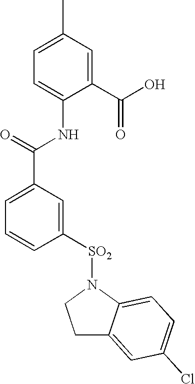 | 1 |
| PHA-556214 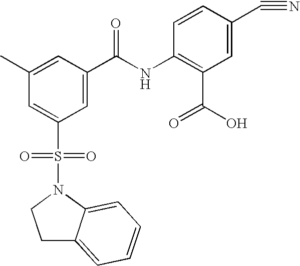 | 1 |
| PHA-556658 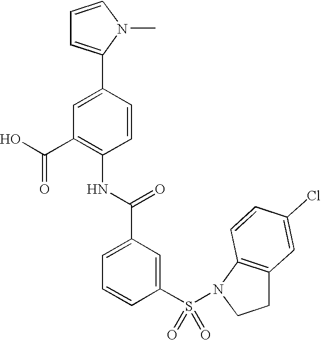 | 8 |
| PHA-556663 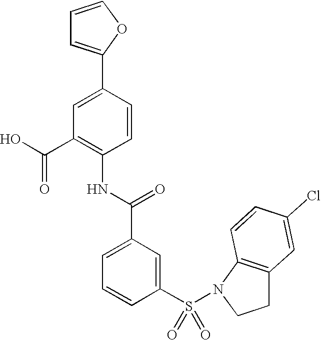 | 8 |
| PHA-561055 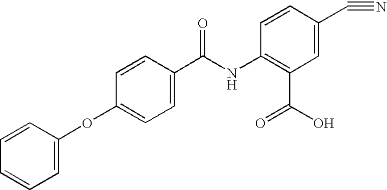 | 1 |
| PHA-562733 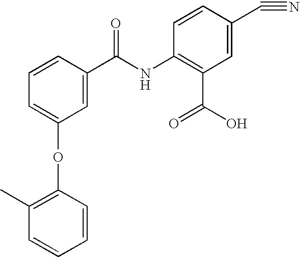 | 0.25 |
| PHA-562862 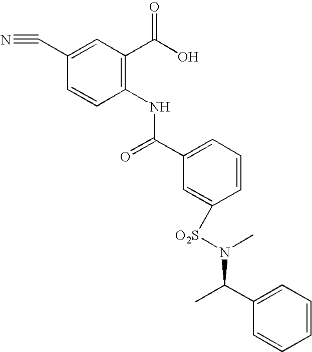 | 4 |
| PHA-562863 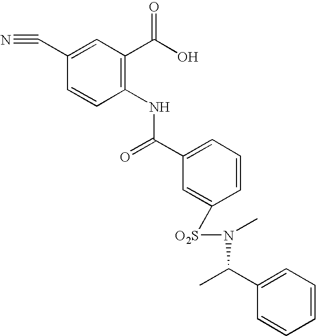 | 2 |
| PHA-563274 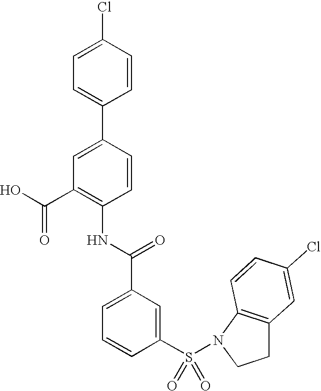 | 2 |
| PHA-563276 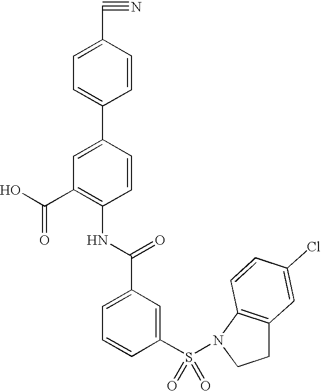 | 2 |
| PHA-563278 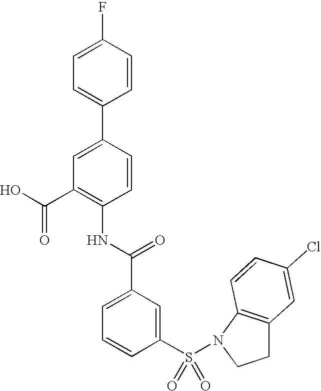 | 2 |
| PHA-563280 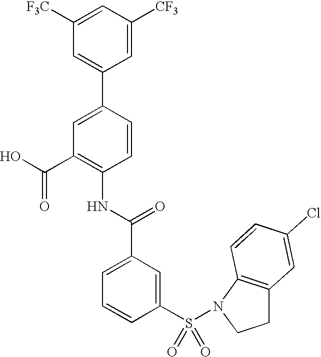 | 1 |
| PHA-563282 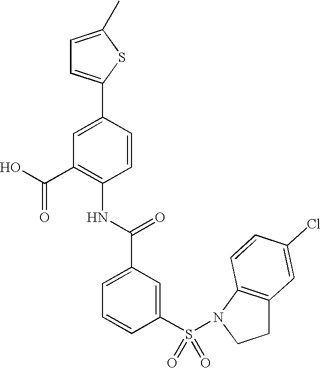 | 1 |
| PHA-563284 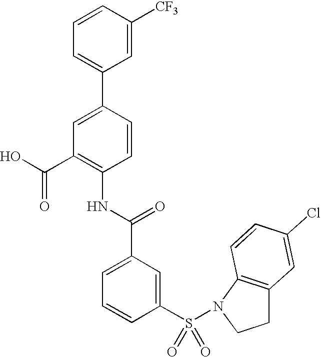 | 2 |
| PHA-563324 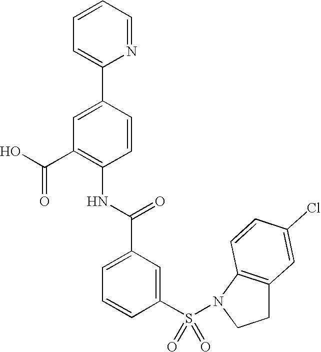 | >128 |
| PHA-564218 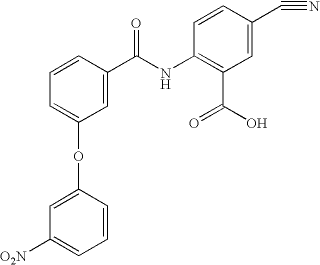 | 1 |
| PHA-566947 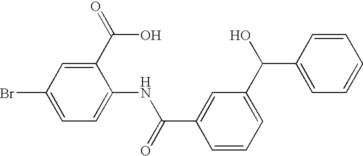 (+)-enantiomer | 0.5 |
| PHA-568196 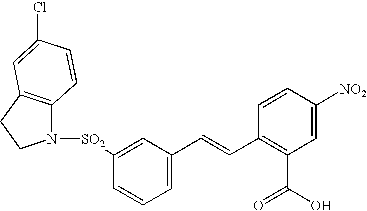 98/2 mixture of trans/cis | 1 |
| PHA-568206 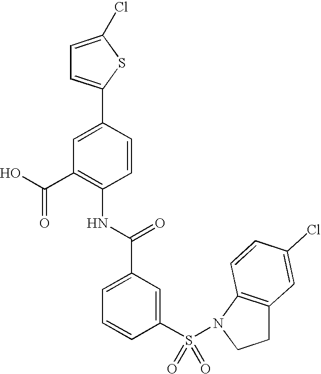 | 2 |
| PHA-568378 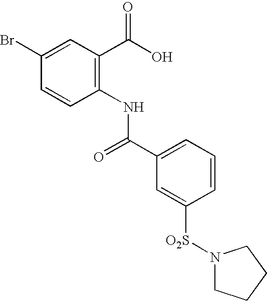 | 2 |
| PHA-568461 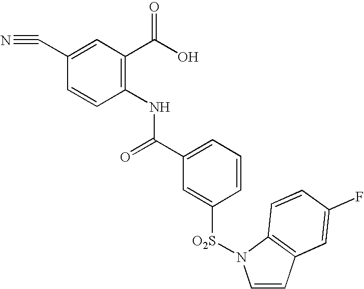 | 0.125 |
| PHA-568907 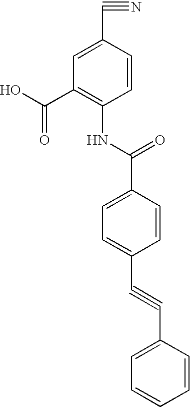 | 8 |
| PHA-569044 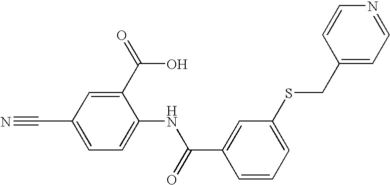 | 0.25 |
| PHA-569064 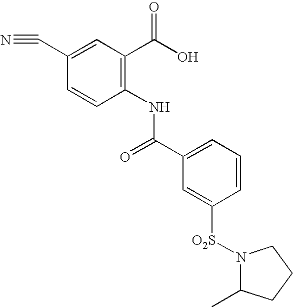 | 1 |
| PHA-569887 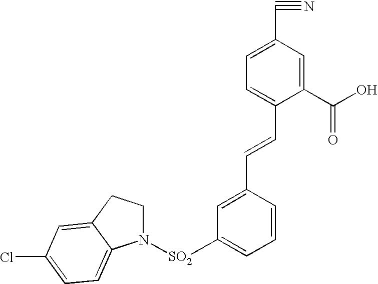 Trans | 0.25 |
| PHA-569977 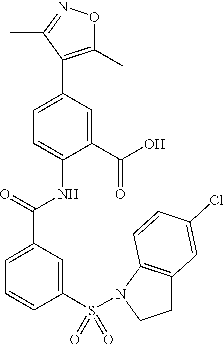 | 16 |
| PHA-570949 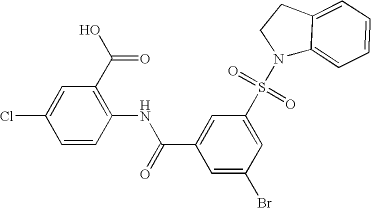 | 1 |
| PHA-571396 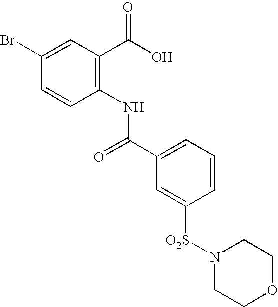 | 4 |
| PHA-571458 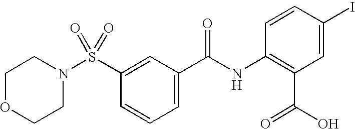 | 4 |
| PHA-615551 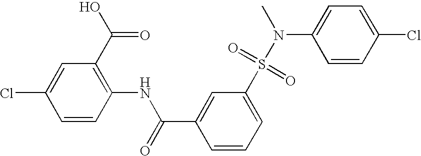 | 1 |
| PHA-630427 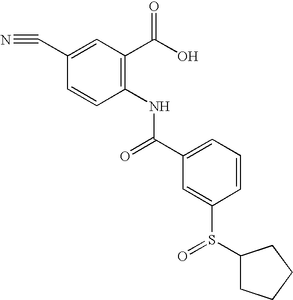 | 4 |
| PHA-630852 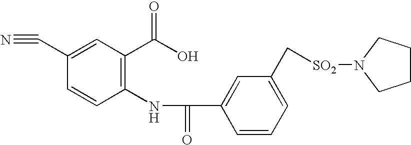 | 4 |
| PHA-630966 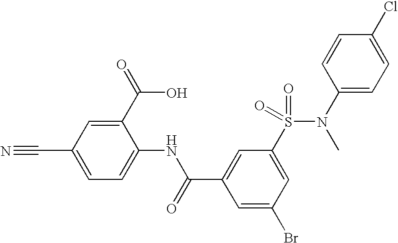 | 0.25 |
| PHA-630989 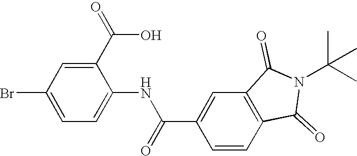 | 4 |
| PHA-662430 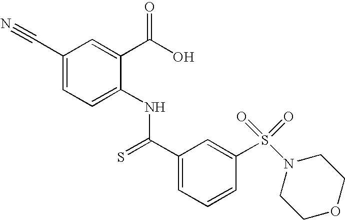 | 1 |
| PHA-662951 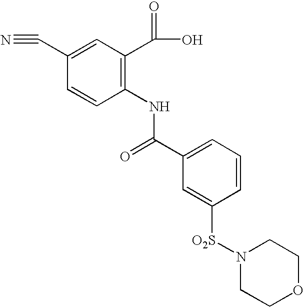 | 32 |
| PHA-666124 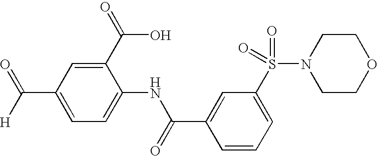 | 32 |
| PHA-681768 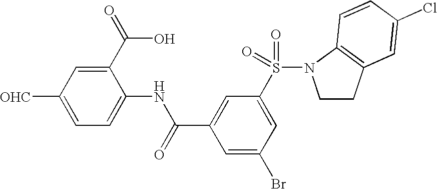 | 1 |
| PHA-686834 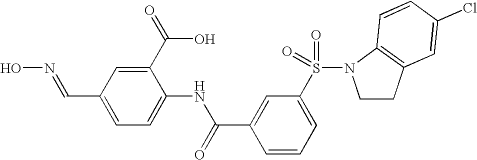 | 4 |
| PHA-707801 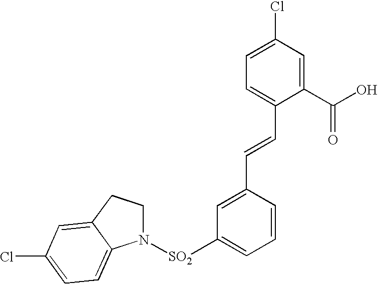 | 4 |
| PHA-708976 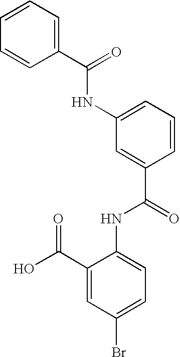 | 32 |
| PHA-708980 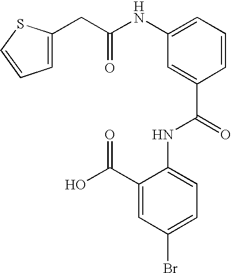 | 16 |
| PHA-708982 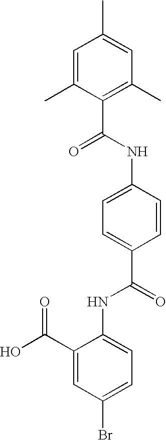 | 128 |
| PHA-708984 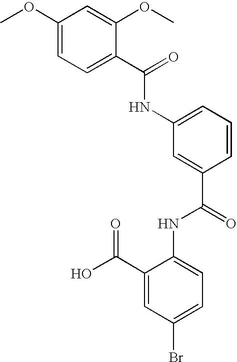 | 32 |
| PHA-708986 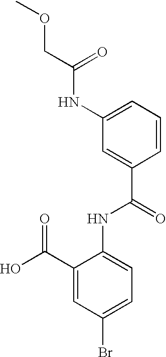 | 64 |
| PHA-708989 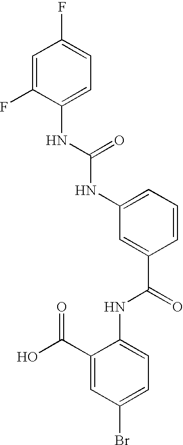 | 8 |
| PHA-708991 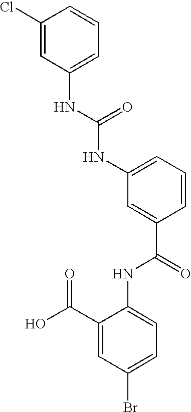 | 4 |
| PHA-708993 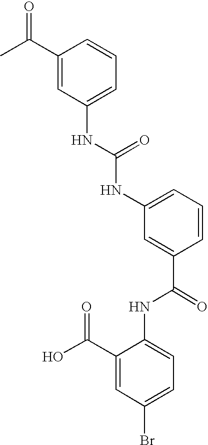 | 4 |
| PHA-500334 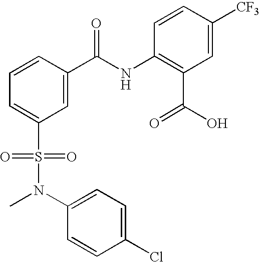 | 16 |
| PHA-502339 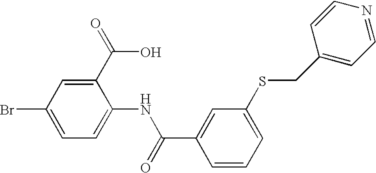 | 2 |
| PHA-502339A 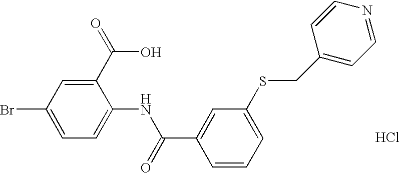 | 8 |
| PHA-509059 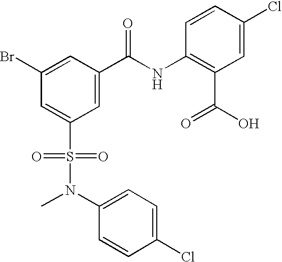 | 0.5 |
| PHA-513535 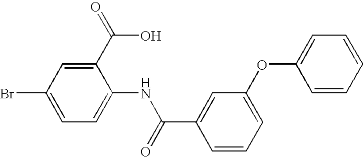 | 2 |
| PHA-513541 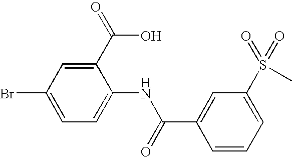 | 64 |
| PHA-515583 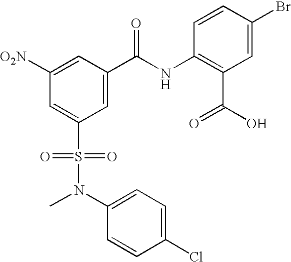 | 8 |
| PHA-516112 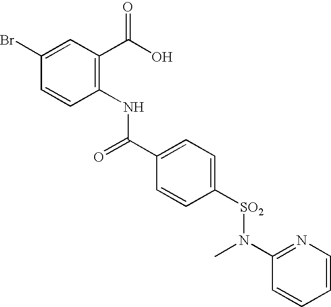 | 8 |
| PHA-516116 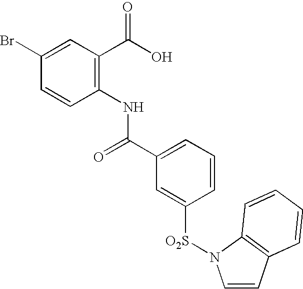 | 0.5 |
| PHA-518226 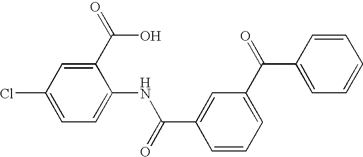 | 2 |
| PHA-520446 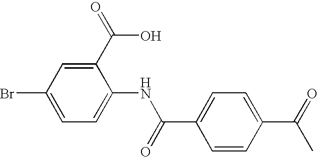 | 16 |
| PHA-520447 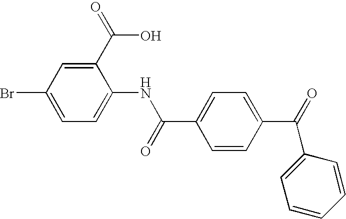 | 1 |
| PHA-520938 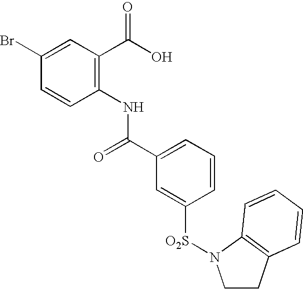 | 1 |
| PHA-521535 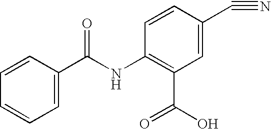 | >128 |
| PHA-522146 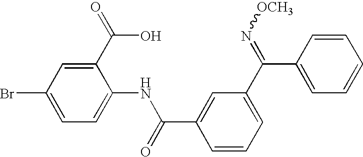 | 0.5 |
| PHA-524524 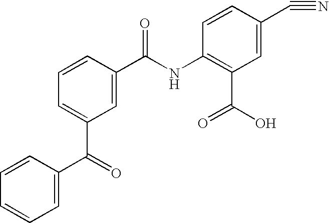 | 1 |
| PHA-526578 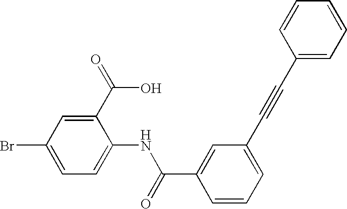 | 2 |
| PHA-530685 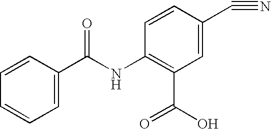 | 32 |
| PHA-530989 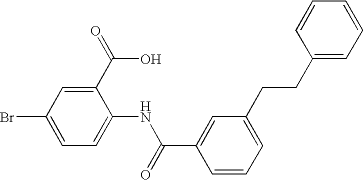 | 4 |
| PHA-543139 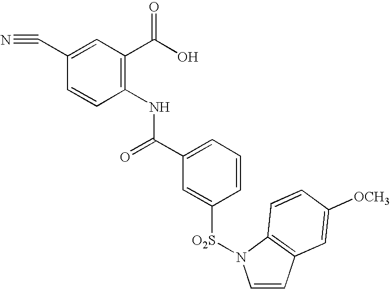 | 0.125 |
| PHA-543141 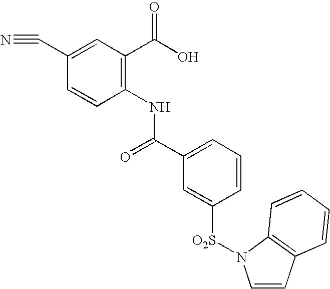 | 0.125 |
| PHA-543681 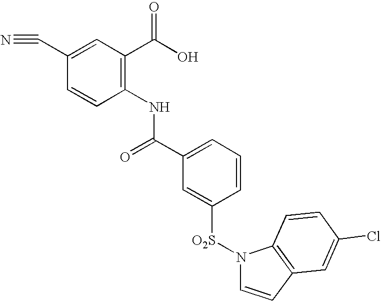 | 0.125 |
| PHA-555027 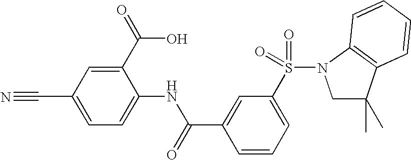 | 1 |
| PHA-556657 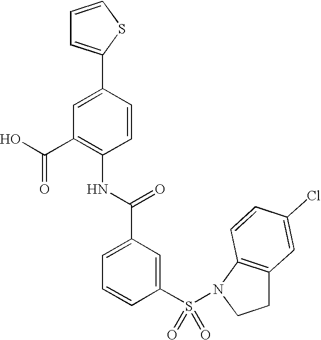 | 2 |
| PHA-556661 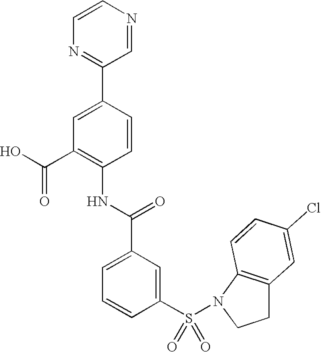 | 8 |
| PHA-557035 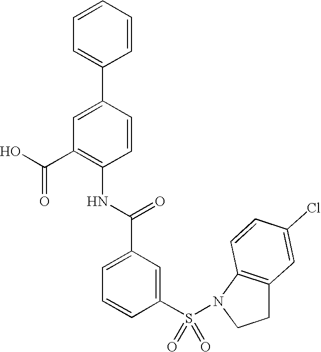 | 4 |
| PHA-562731 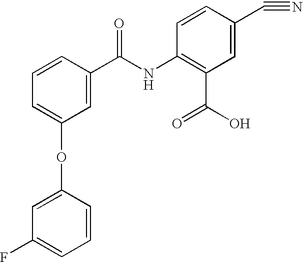 | 1 |
| PHA-562745 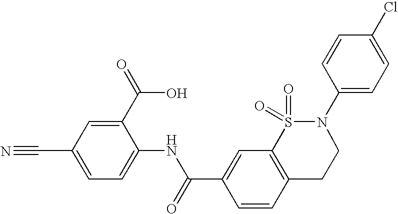 | 0.25 |
| PHA-563275 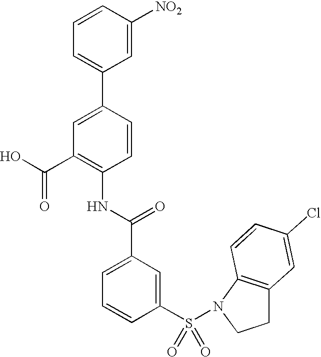 | 2 |
| PHA-563277 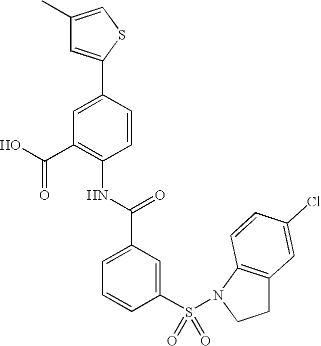 | 2 |
| PHA-563279 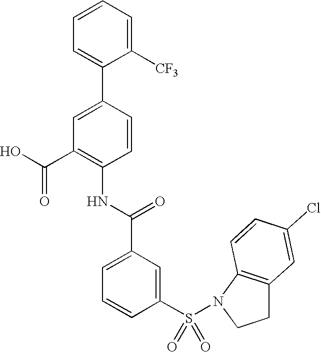 | 0.5 |
| PHA-563281 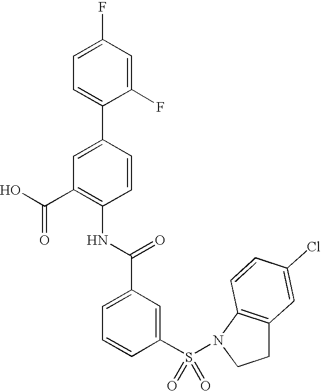 | 1 |
| PHA-563283 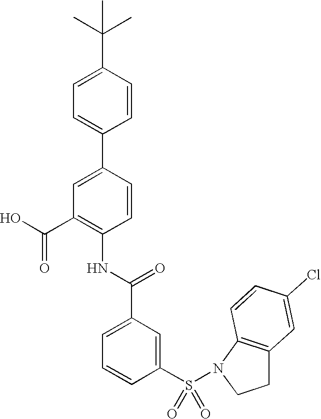 | 16 |
| PHA-563285 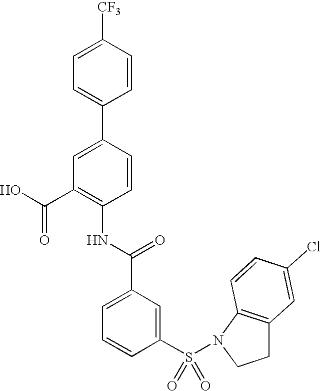 | 2 |
| PHA-564215 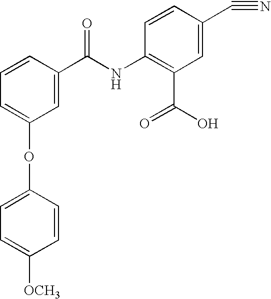 | 0.5 |
| PHA-564750 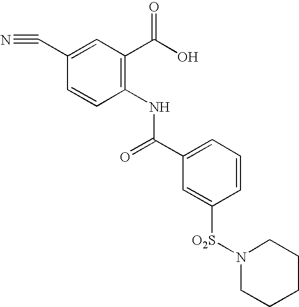 | 0.25 |
| PHA-566948 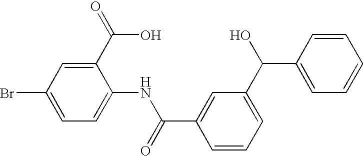 (−)-enantiomer | 1 |
| PHA-568197 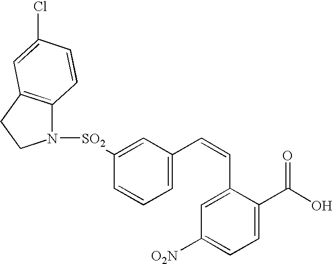 6.3/93.7 trans/cis | 16 |
| PHA-568205 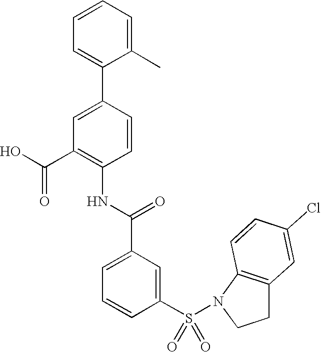 | 2 |
| PHA-568376 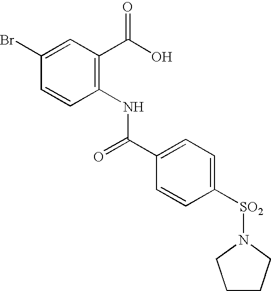 | 16 |
| PHA-568420 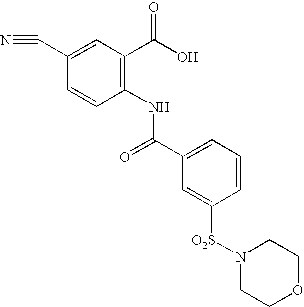 | 0.5 |
| PHA-568422 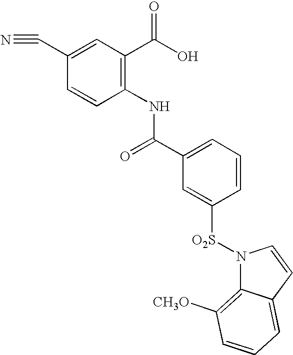 | 0.125 |
| PHA-568424 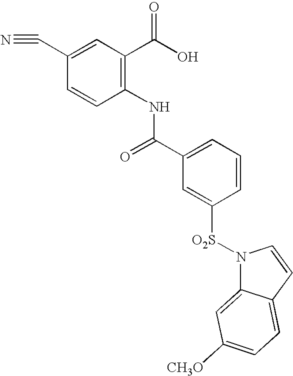 | 1 |
| PHA-568425 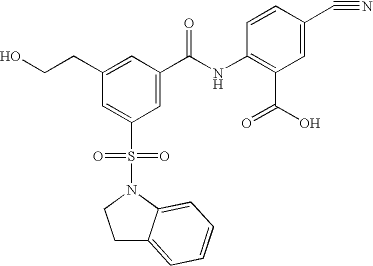 | 8 |
| PHA-568906 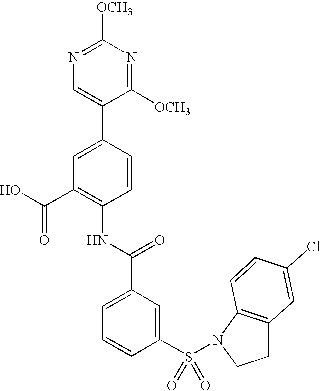 | 8 |
| PHA-569044A 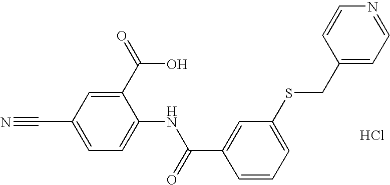 | 0.5 |
| PHA-569077 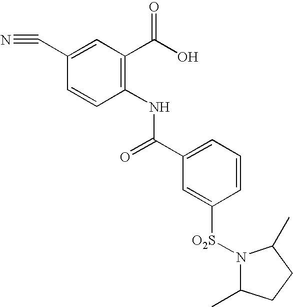 | 1 |
| PHA-569885 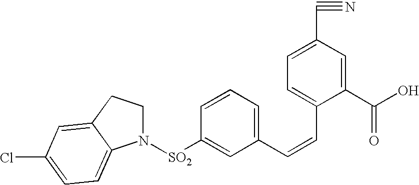 This is 97.9/2.1 cis/trans | 16 |
| PHA-569974 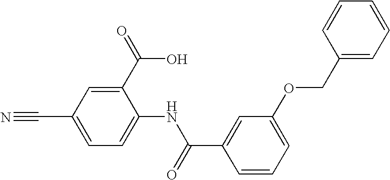 | 1 |
| PHA-570008 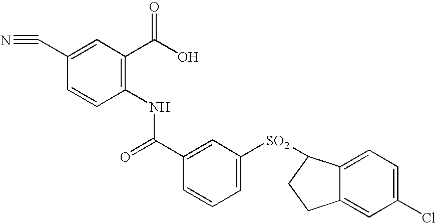 | 0.125 |
| PHA-570042 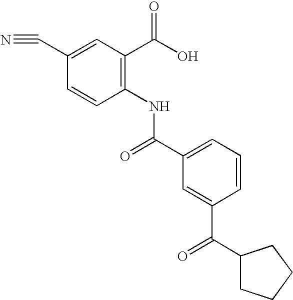 | 2 |
| PHA-571395 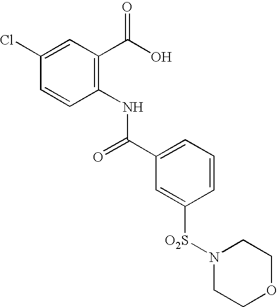 | 4 |
| PHA-571397 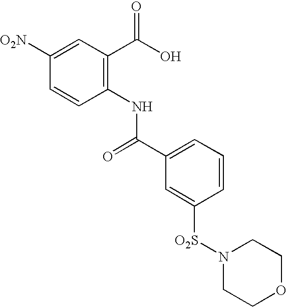 | 4 |
| PHA-610938 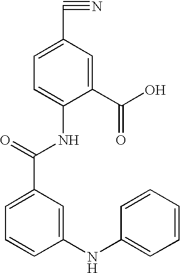 | 1 |
| PHA-630368 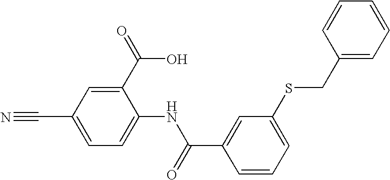 | 0.5 |
| PHA-630726 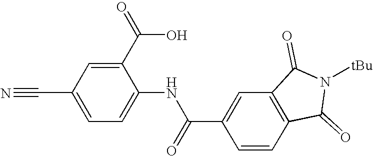 | 4 |
| PHA-630965 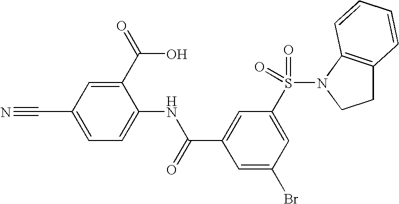 | 0.25 |
| PHA-631082 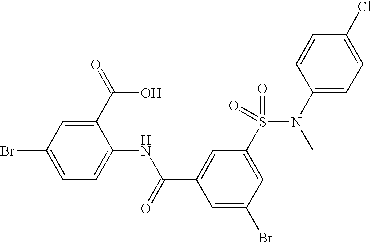 | 0.25 |
| PHA-662250 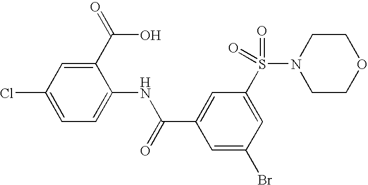 | 1 |
| PHA-662431 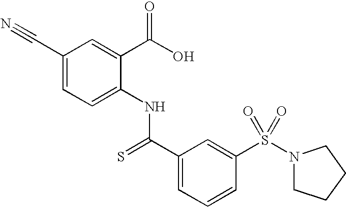 | 1 |
| PHA-664658 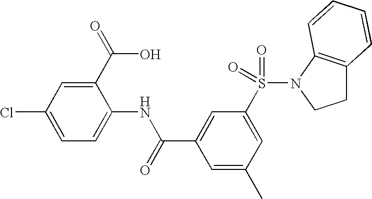 | 4 |
| PHA-670083 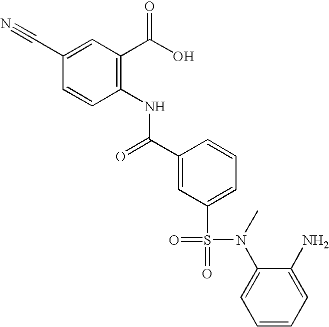 | 0.5 |
| PHA-682996 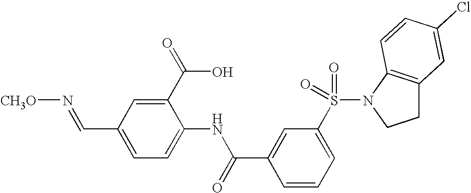 | 64 |
| PHA-687511 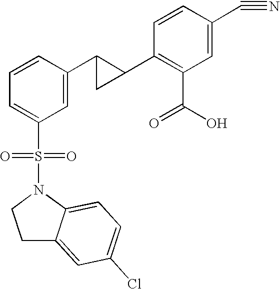 is >99 trans | 4 |
| PHA-708923 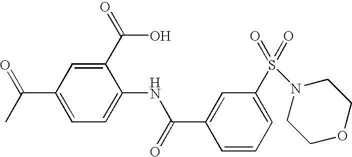 | 32 |
| PHA-708978 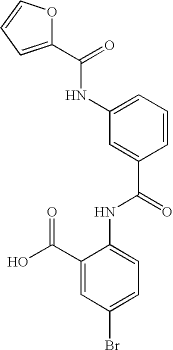 | 32 |
| PHA-708981 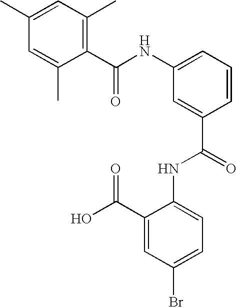 | 16 |
| PHA-708983 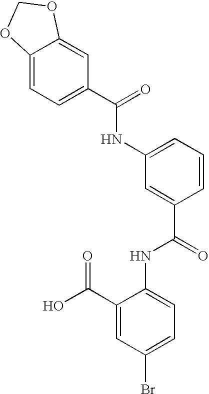 | 32 |
| PHA-708985 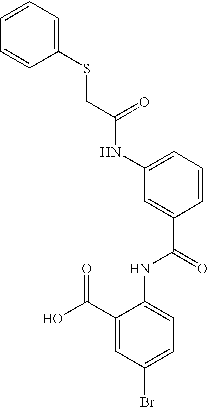 | 8 |

| | SA 9218 |
|---|---|
| Compound No., Structure | MIC |
| PHA-708995 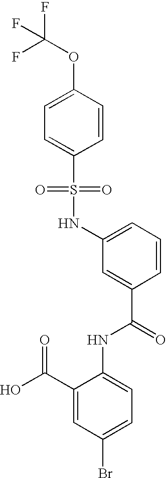 | 0.125 |
| PHA-708997 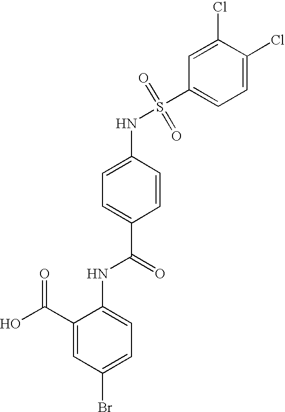 | 8 |
| PHA-713387 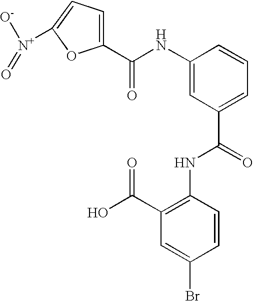 | 128 |
| PHA-713394 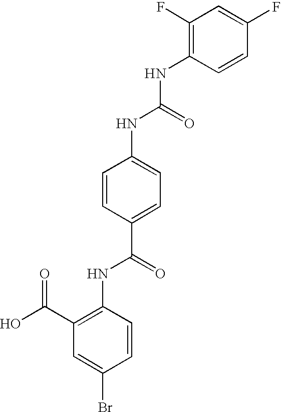 | 128 |
| PHA-713398 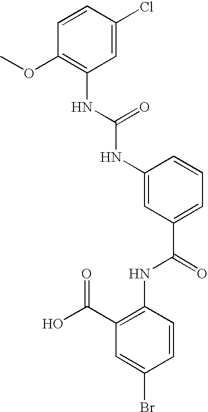 | 4 |
| PHA-713400 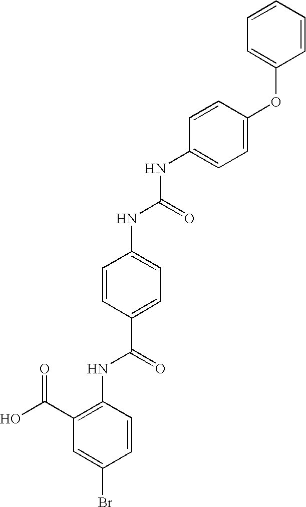 | 16 |
| PHA-713403 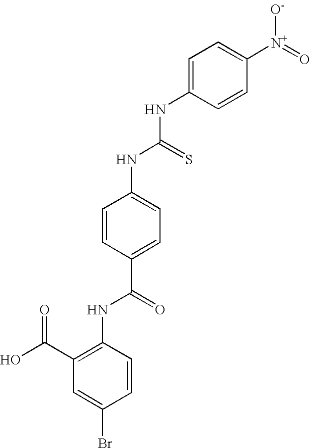 | 64 |
| PHA-713406 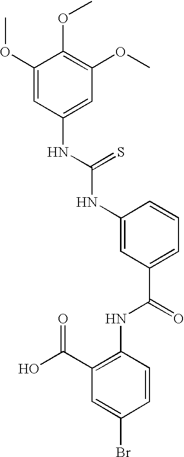 | 64 |
| PHA-713408 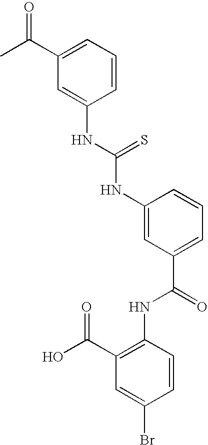 | 64 |
| PHA-713410 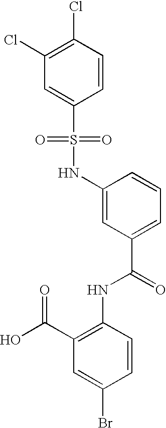 | 1 |
| PHA-717196 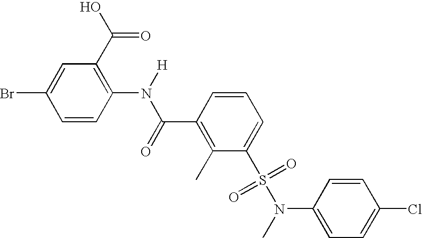 | 4 |
| PHA-728844 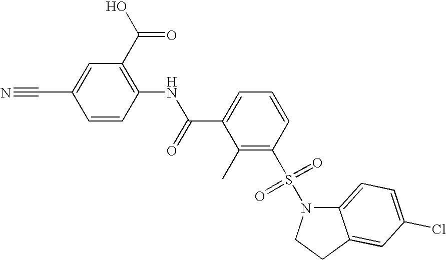 | 0.25 |
| PNU-263533 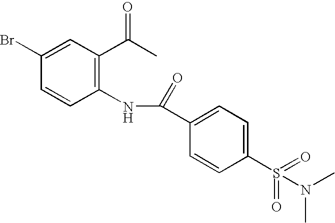 | |
| PNU-271584 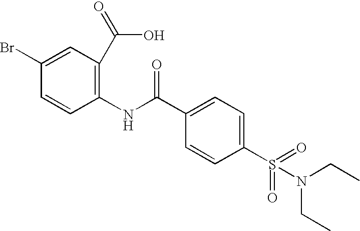 | |
| PNU-276296 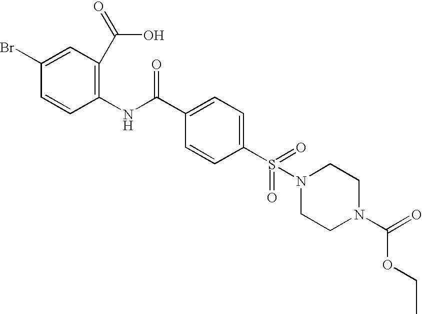 | |
| PNU-276637 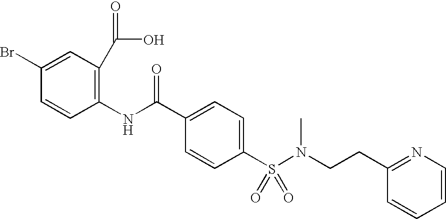 | |
| PNU-276670 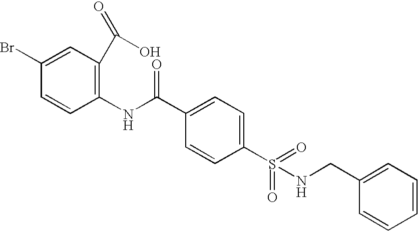 C21H17BrN2O5S Exact wt. 488.0042 | |
| PNU-276817 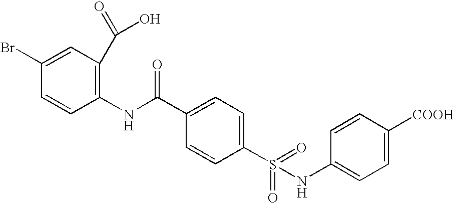 | 4 |
| PNU-276854 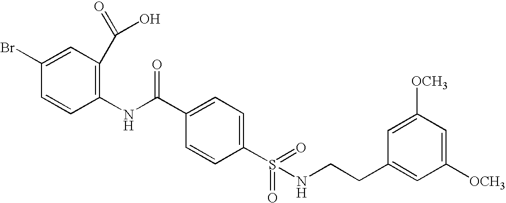 | |
| PNU-276933 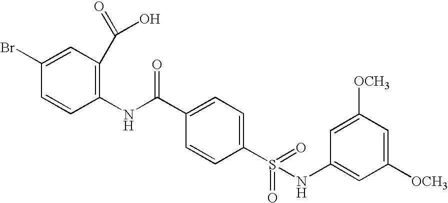 | |
| PNU-276988 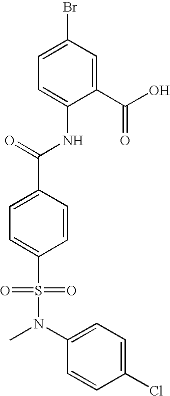 | 16 |
| PNU-277231 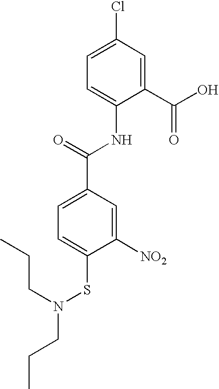 | 1 |
| PNU-280772 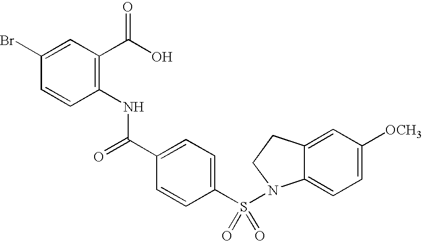 | |
| PNU-283076 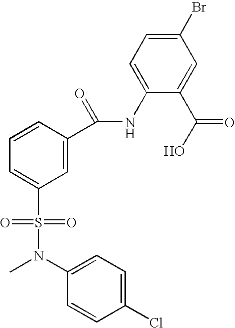 | 1 |
| PNU-283599 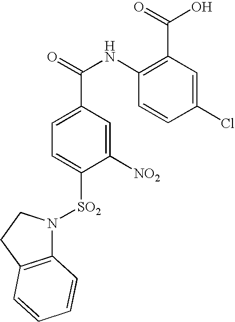 | 1 |
| PNU-283603A 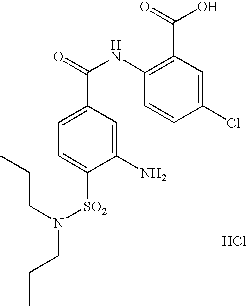 | 16 |
| PNU-288969 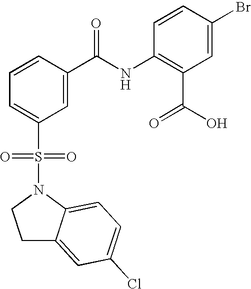 | 0.25 |
| PNU-290821 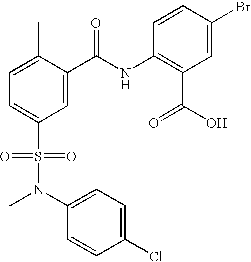 | 64 |
| PNU-290877 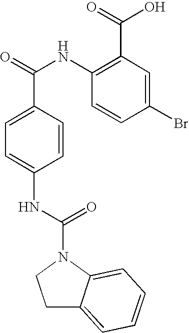 See Comments | >128 |
| PNU-290905 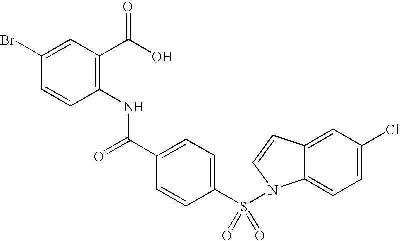 | 1 |
| PNU-290906 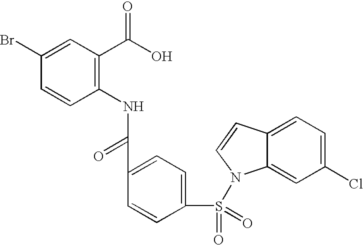 | 1 |
| PNU-291061 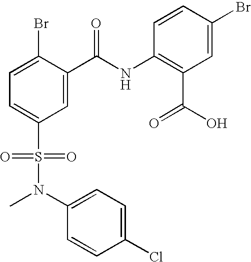 | 16 |
| PNU-291410 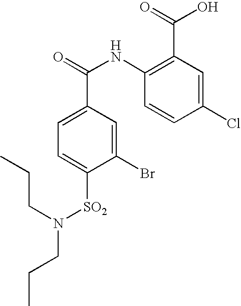 | 4 |
| PNU-291570 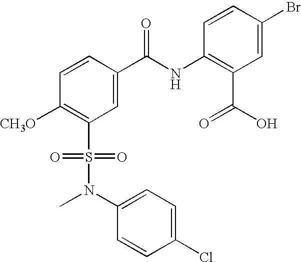 | 8 |
| PNU-291571 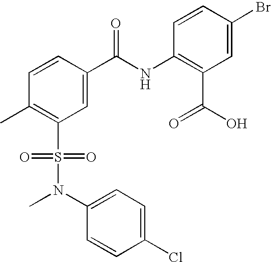 | 0.5 |
| PNU-292070 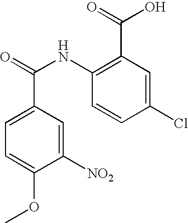 | 2 |
| PNU-293032 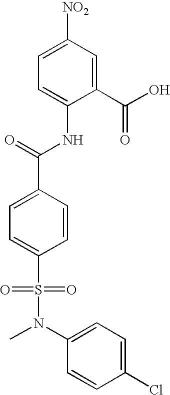 | 16 |
| PNU-293905 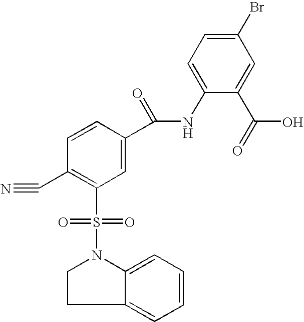 | 8 |
| PHA-630331 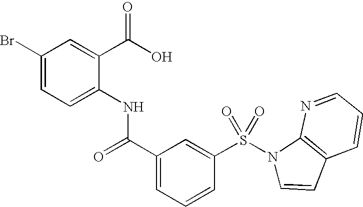 | 2 |
| PNU-293795 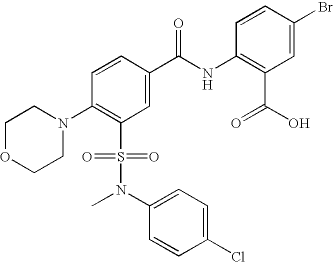 | 32 |
| PNU-294595 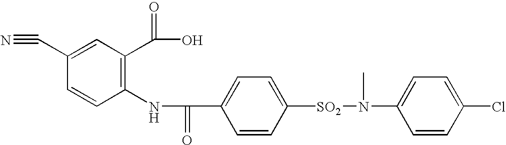 | 16 |
| PHA-630330 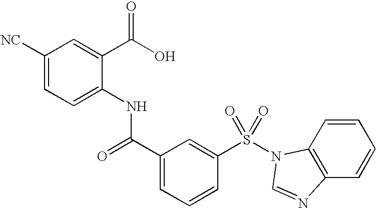 | 0.5 |
| PHA-708988 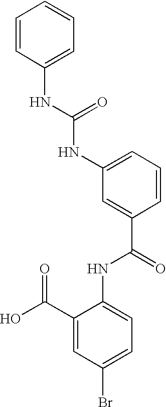 | 32 |
| PHA-708990 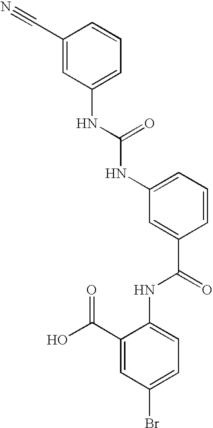 | 8 |
| PHA-708992 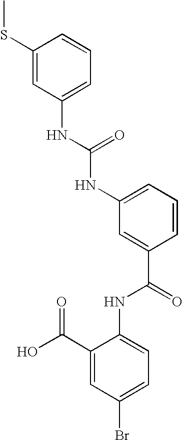 | 4 |
| PHA-708994 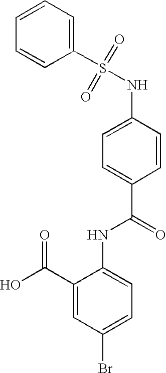 | 8 |
| PHA-708996 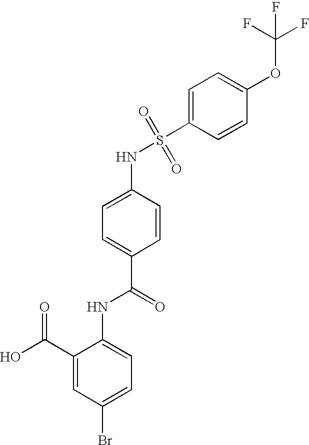 | 16 |
| PHA-713386 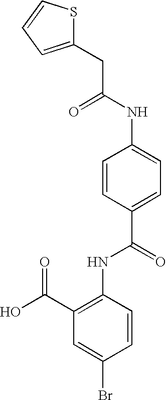 | 128 |
| PHA-713388 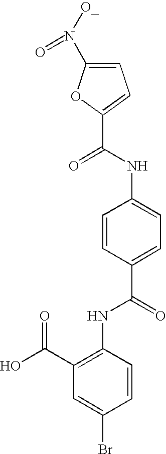 | 16 |
| PHA-713396 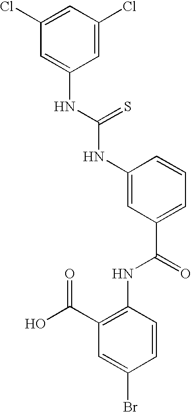 | 8 |
| PHA-713399 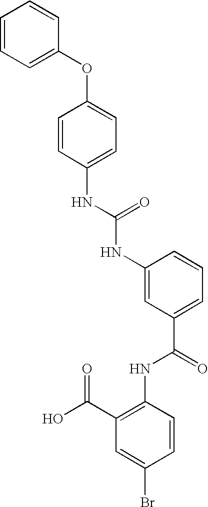 | 16 |
| PHA-713401 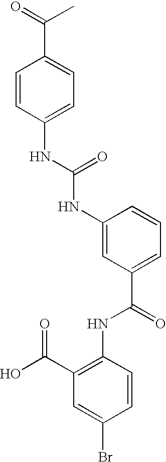 | 32 |
| PHA-713405 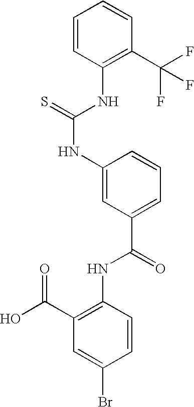 | 128 |
| PHA-713407 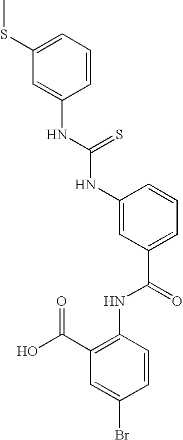 | 32 |
| PHA-713409 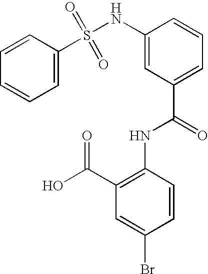 | 1 |
| PHA-713411 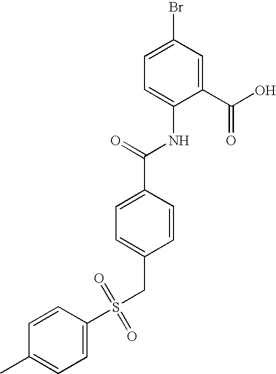 | 32 |
| PHA-719201 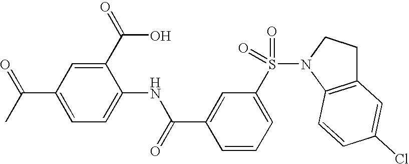 | 2 |
| PHA-735753 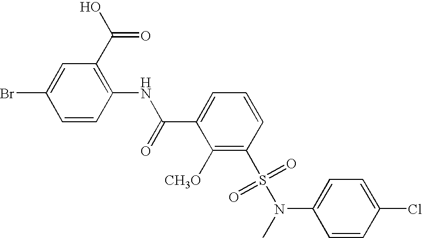 | 16 |
| PNU-268205 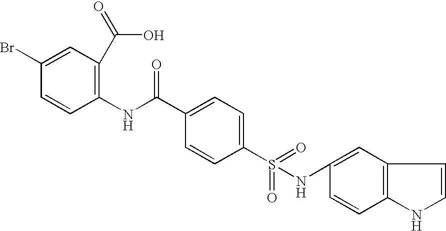 | |
| PNU-275747 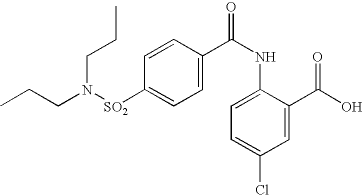 | |
| PNU-276301 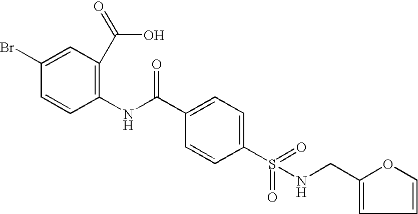 | |
| PNU-276638 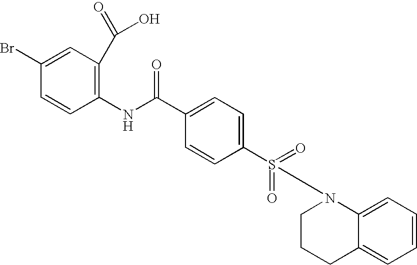 C18H17BrN2O6S Exact wt. 467.9991 | |
| PNU-276728 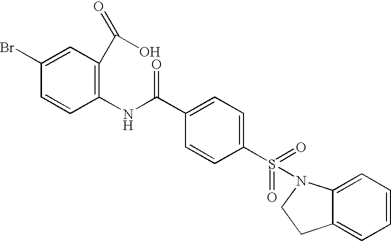 C22H17BrN2O5S Exact wt. 500.0042 | |
| PNU-276770 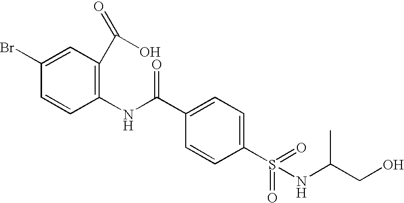 C17H17BrN2O6S Exact wt. 455.9991 | |
| PNU-276818 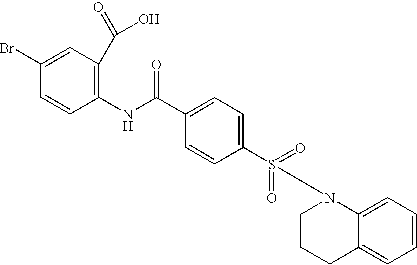 | |
| PNU-276913 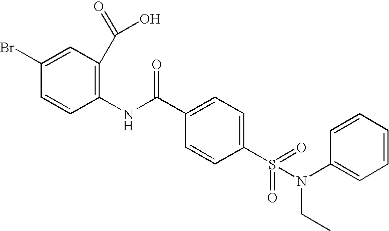 | |
| PNU-276952 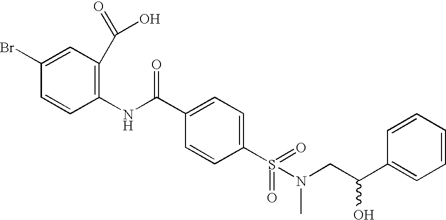 racemic | |
| PNU-280727 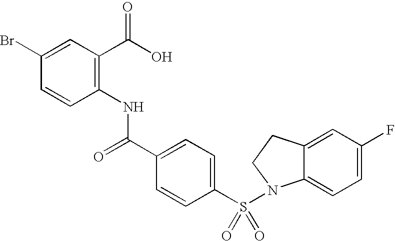 | |
| PNU-282958 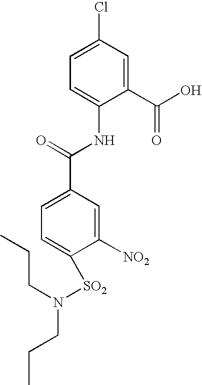 | |
| PNU-283318 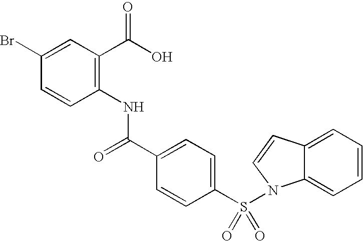 | 0.125 |
| PNU-283371 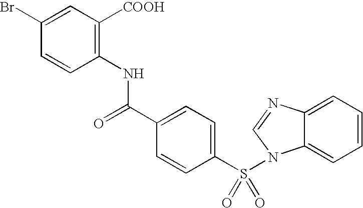 | 4 |
| PNU-283601A 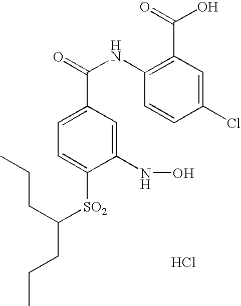 | 32 |
| PNU-283604 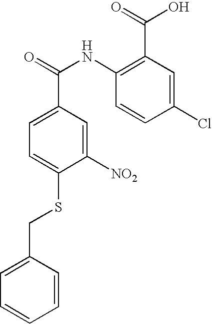 | 4 |
| PNU-289815 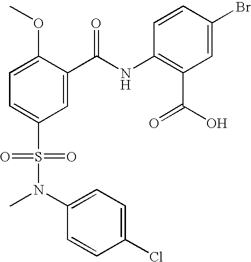 | 8 |
| PNU-290882 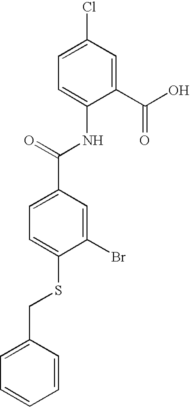 | 1 |
| PNU-291010 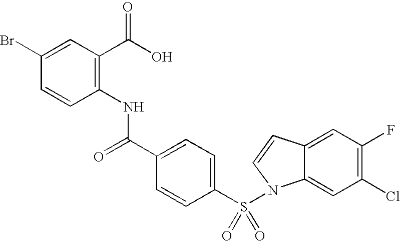 | 1 |
| PNU-291011 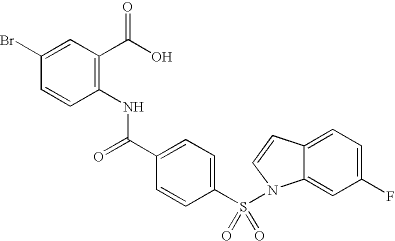 | 0.25 |
| PNU-291129 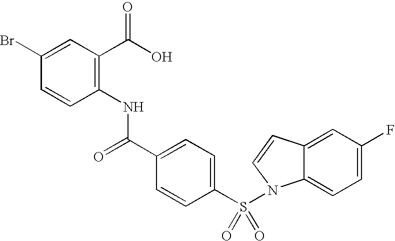 | 0.5 |
| PNU-291130 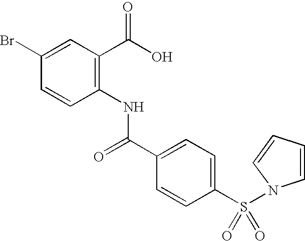 | 4 |
| PNU-291408 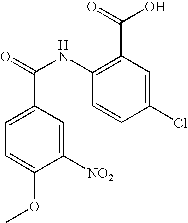 | 32 |
| PNU-291517 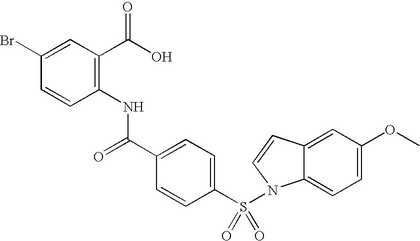 | 2 |
| PNU-291679 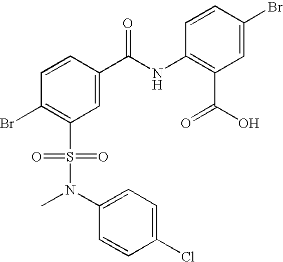 | 1 |
| PNU-292379 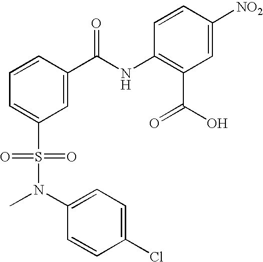 | 0.5 |
| PNU-293049 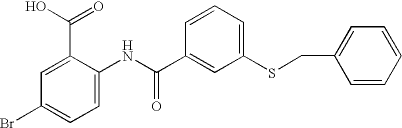 | 4 |

TABLE 2
Activity Data

| | SA 9218 | | SA 9218 |
|---|---|---|---|
| Compound No., Structure | MIC | Compound No., Structure | MIC |
| 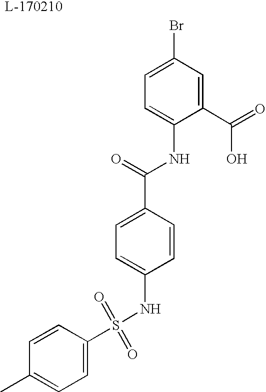 | 16 | 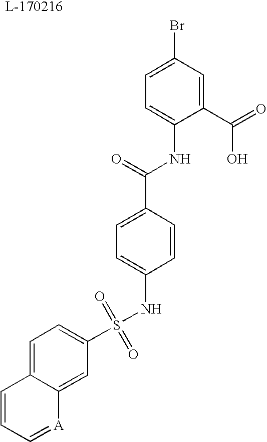 | |
| 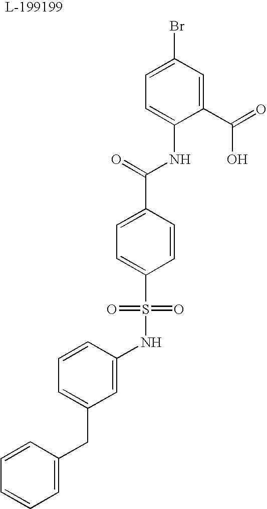 | | 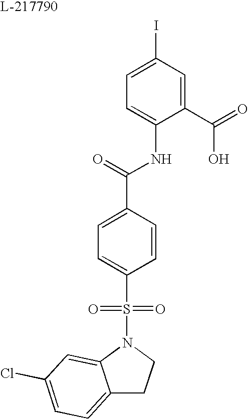 | 4 |
| 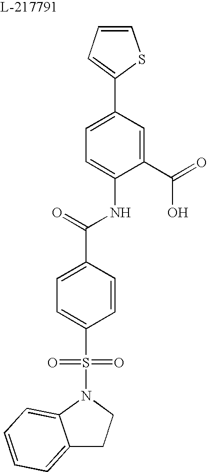 | 4 | 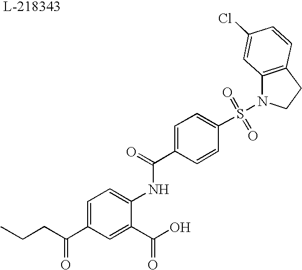 | 16 |
| 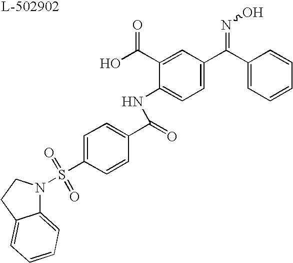 | 128 | 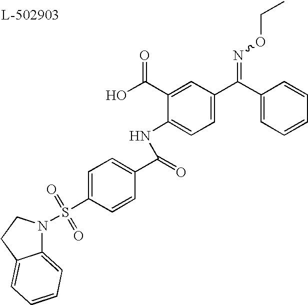 | 16 |
| 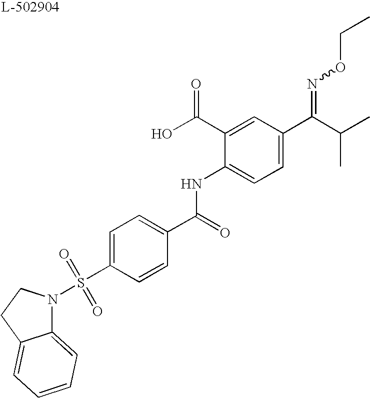 | 64 | 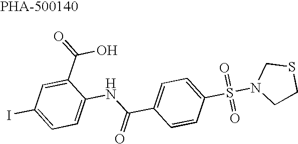 | 32 |
| 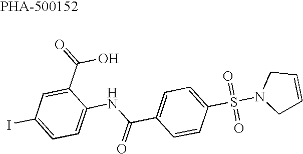 | 32 | 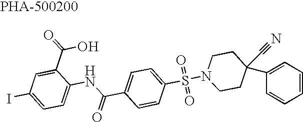 | 4 |
| 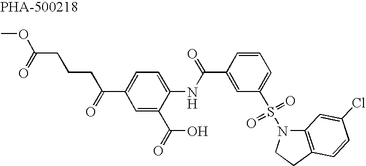 | 64 | 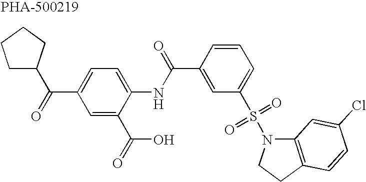 | 32 |
| 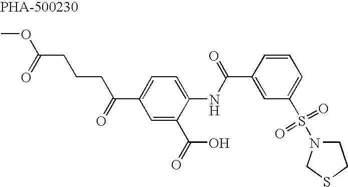 | >128 | 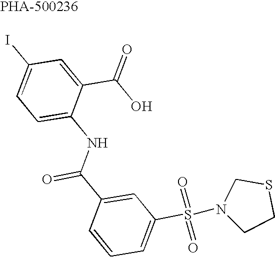 | 8 |
| 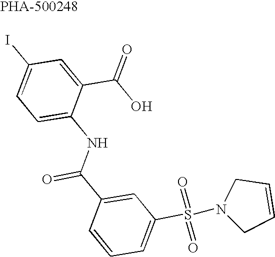 | 8 | 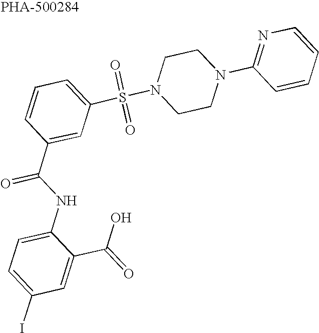 | 32 |
| 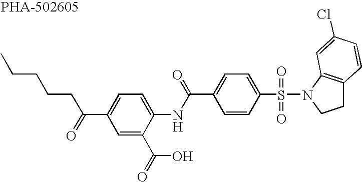 | 8 | 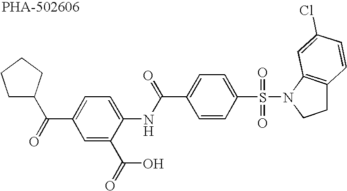 | 16 |
| 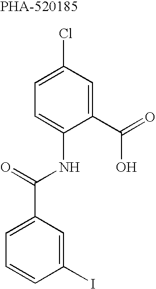 | 8 | 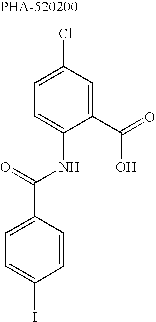 | 2 |
| 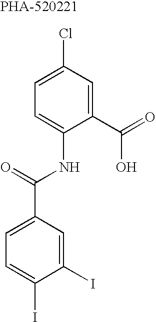 | 2 | 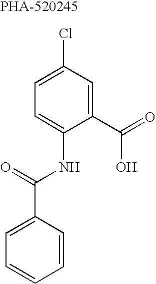 | 128 |
| 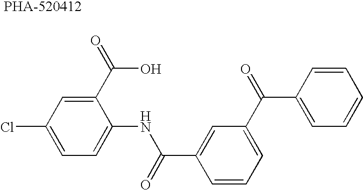 | 4 | 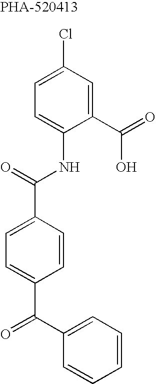 | 4 |
| 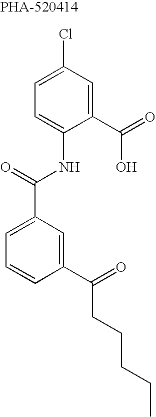 | 8 | 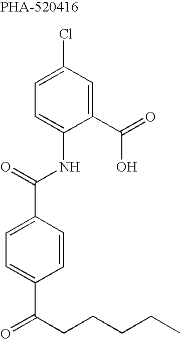 | 4 |
| 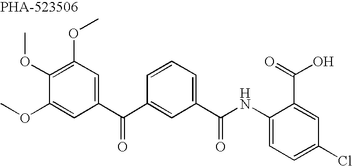 | 32 | 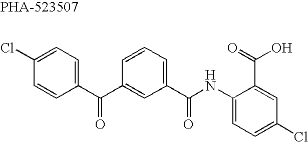 | 4 |
| 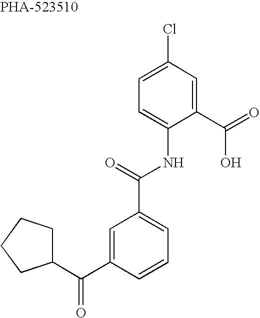 | 8 | 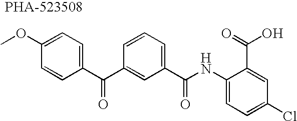 | 8 |
| 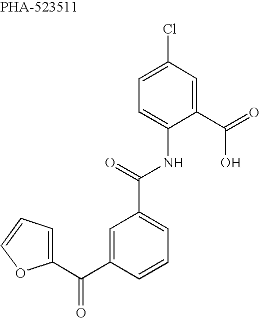 | 8 | 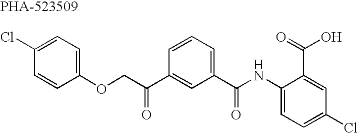 | 8 |
| 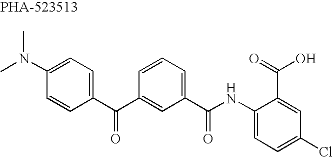 | 4 | 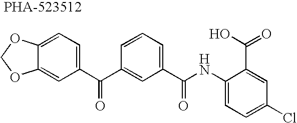 | 4 |
| 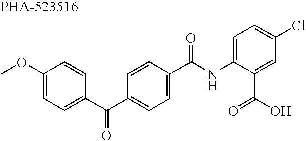 | 2 | 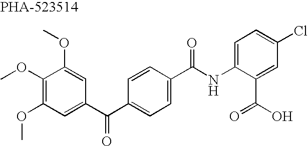 | 4 |
| 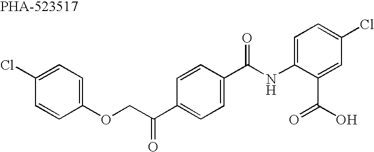 | 4 | 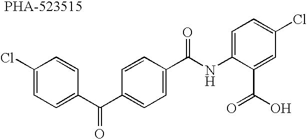 | 4 |
| 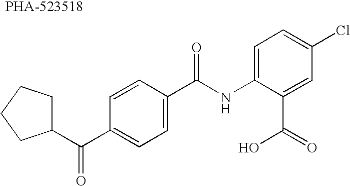 | 8 | 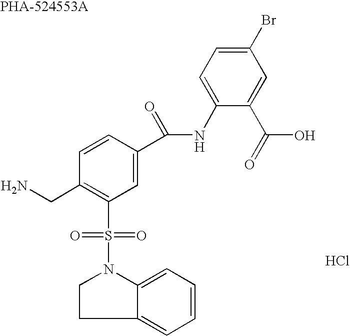 | >128 |
| 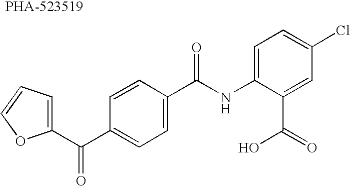 | 4 | 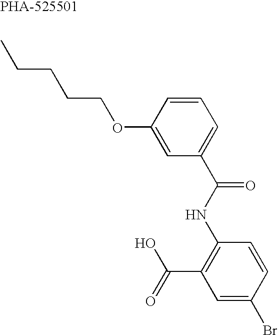 | 8 |
| 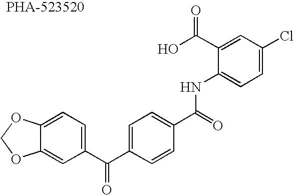 | 2 | 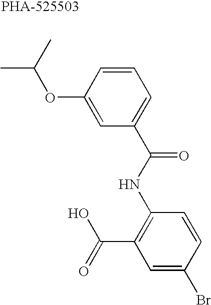 | 2 |
| 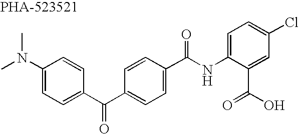 | 16 | 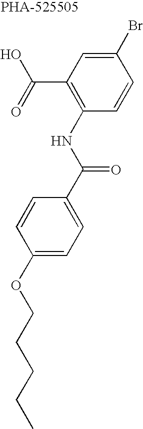 | 16 |
| 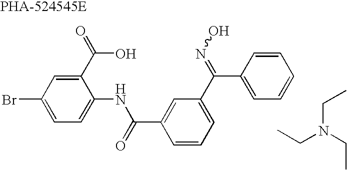 | 0.5 | 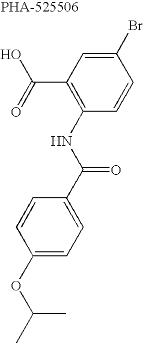 | 64 |
| 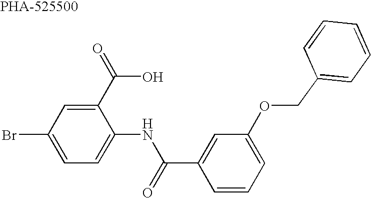 | 4 | 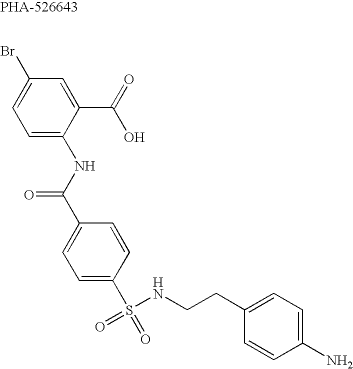 | 64 |
| 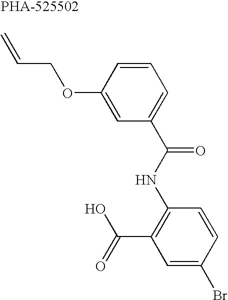 | 8 | 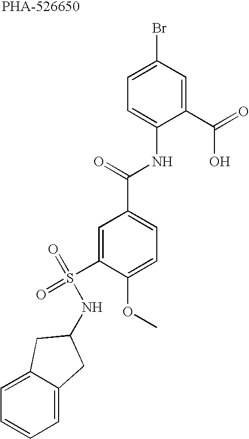 | 2 |
| 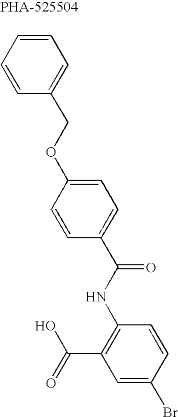 | 16 | 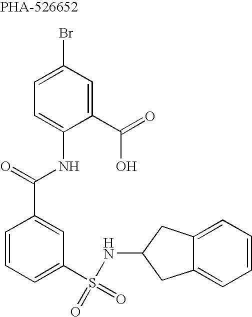 | 1 |
| 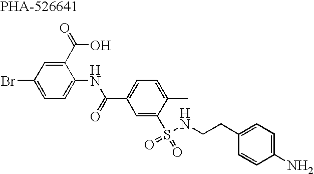 | 8 | 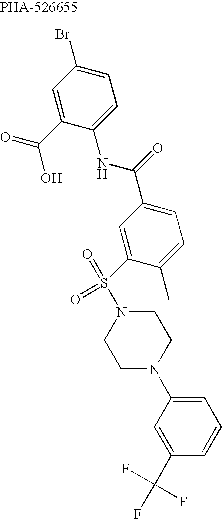 | 16 |
| 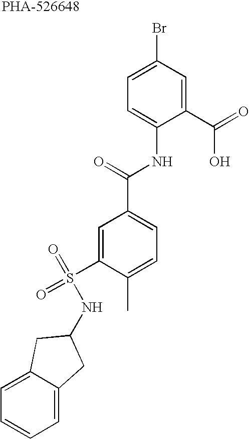 | 0.25 | 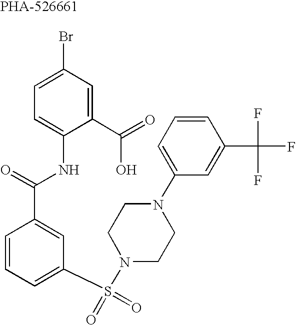 | 16 |
| 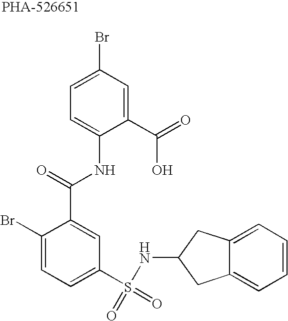 | 0.25 | 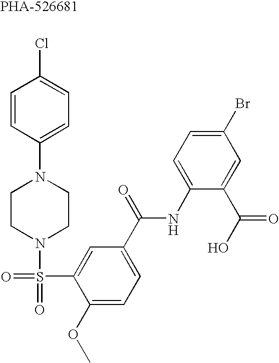 | 8 |
| 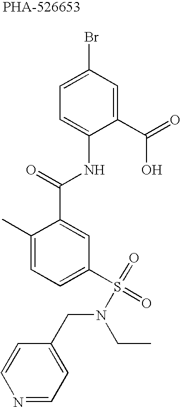 | >128 | 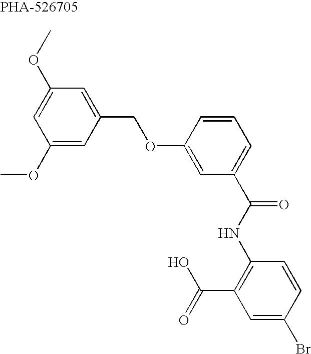 | 16 |
| 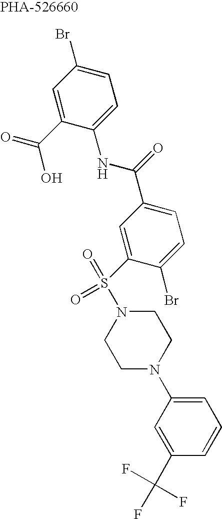 | 8 | 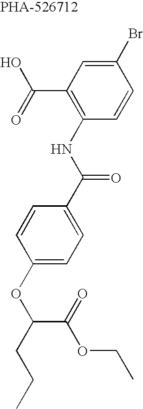 | 64 |
| 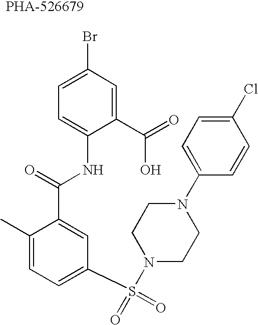 | 32 | 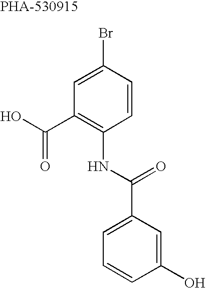 | 32 |
| 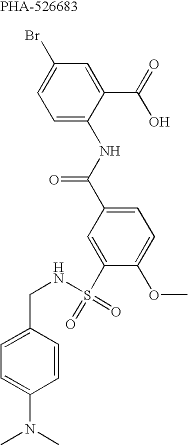 | >128 | 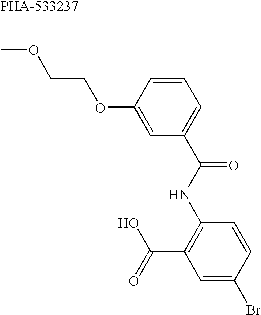 | 16 |
| 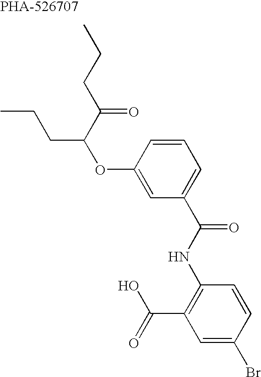 | 2 | 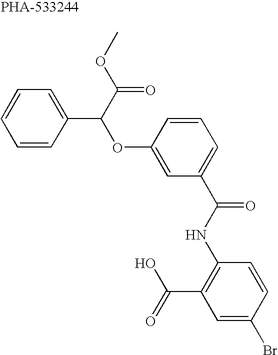 | 4 |
| 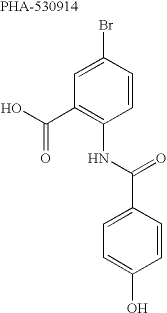 | 32 | 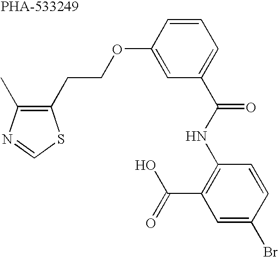 | 8 |
| 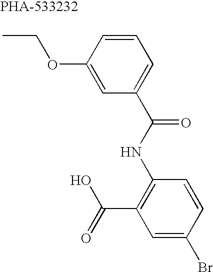 | 64 | 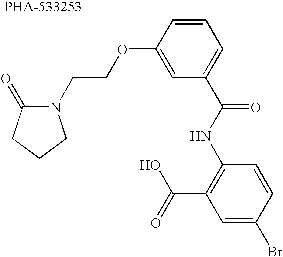 | 32 |
| 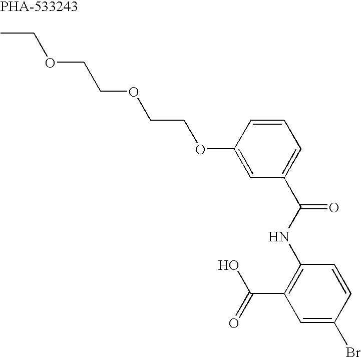 | 32 | 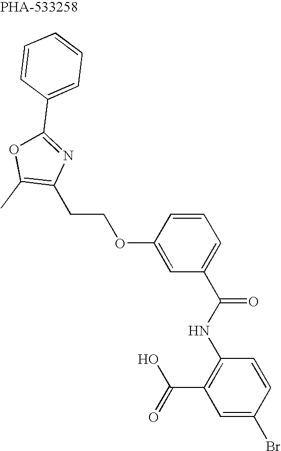 | 32 |
| 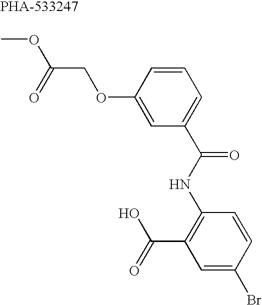 | 32 | 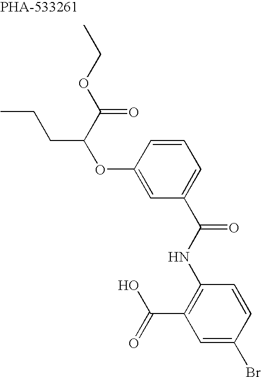 | 8 |
| 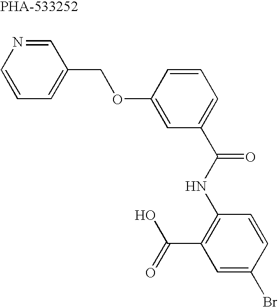 | 32 | 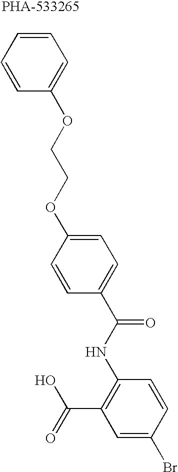 | 128 |
| 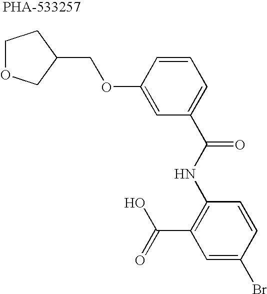 | 16 | 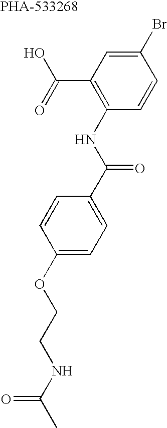 | 64 |
| 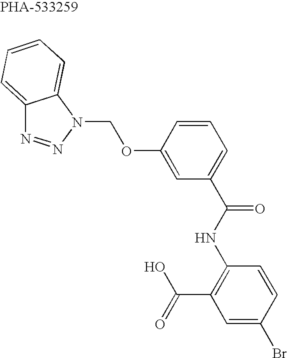 | 32 | 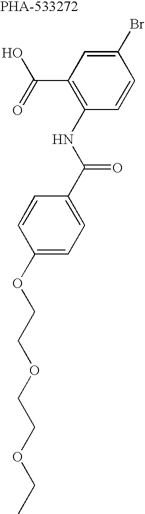 | 128 |
| 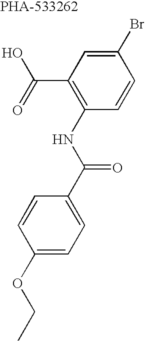 | 64 | 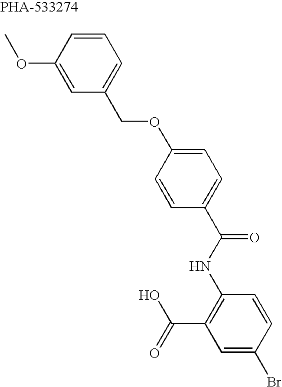 | 8 |
| 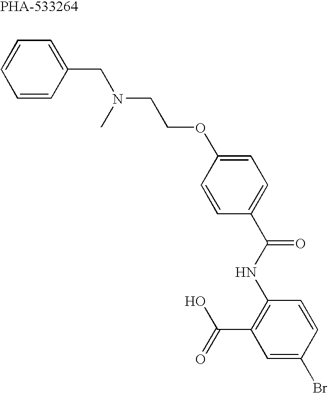 | 128 | 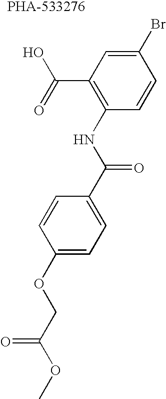 | 64 |
| 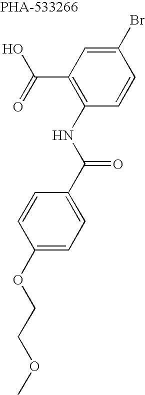 | 128 | 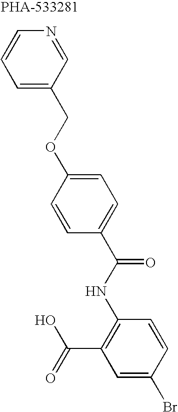 | 64 |
| 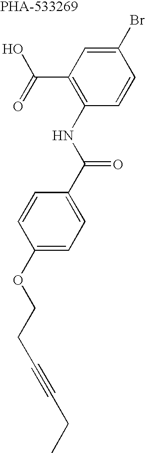 | 16 | 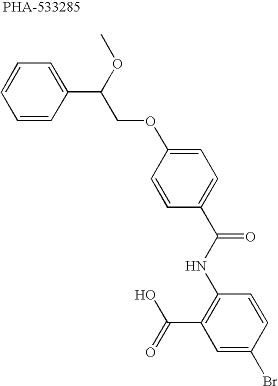 | 64 |
| 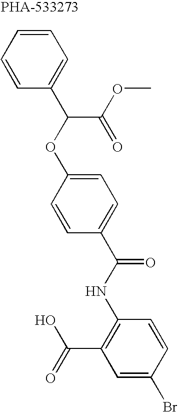 | 64 | 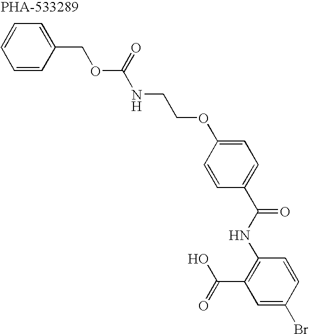 | 64 |
| 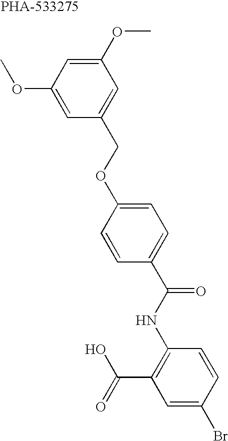 | 8 | 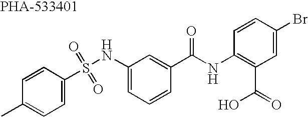 | 0.5 |
| 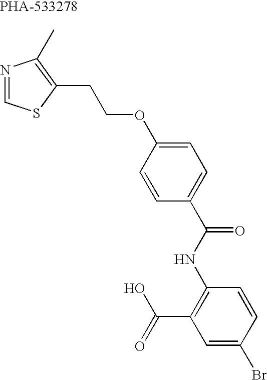 | 32 | 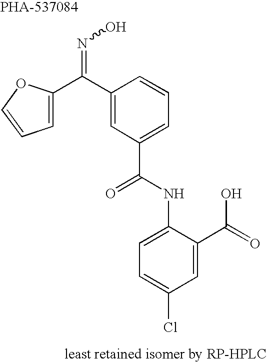 | 2 |
| 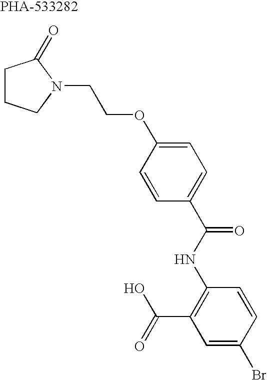 | >128 | 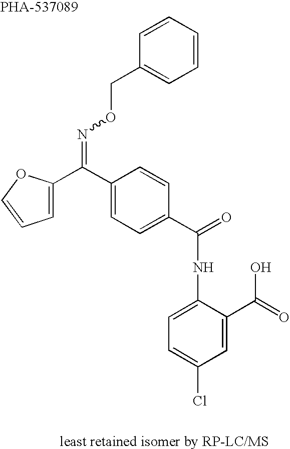 | 32 |
| 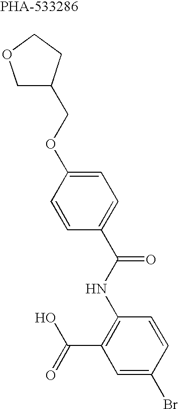 | 128 | 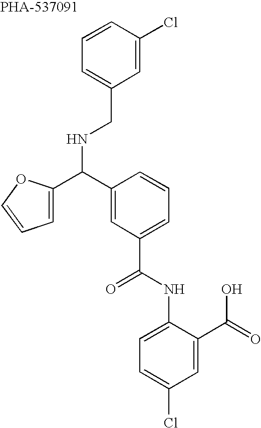 | 8 |
| 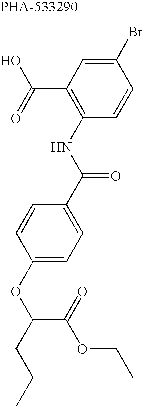 | 64 | 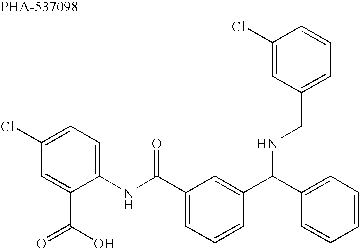 | 16 |
| 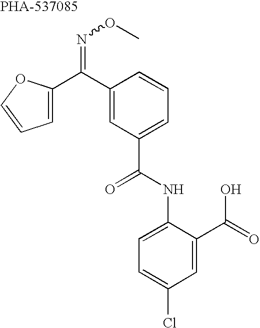 | 16 | 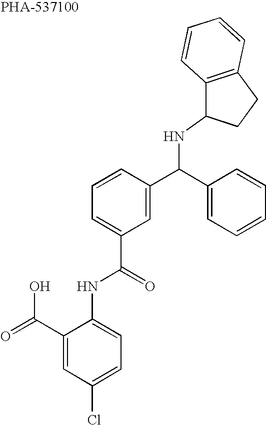 | 16 |
| 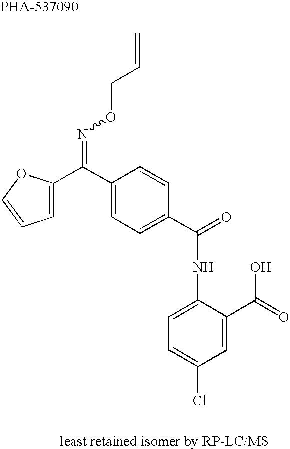 | 32 | 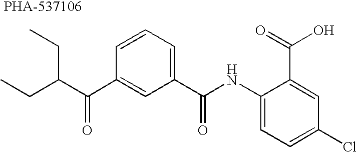 | 8 |
| 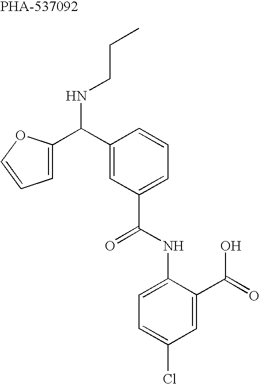 | 16 | 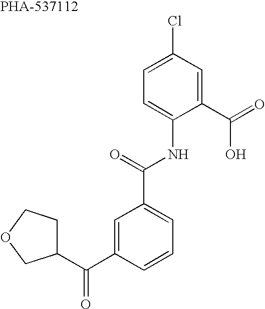 | 128 |
| 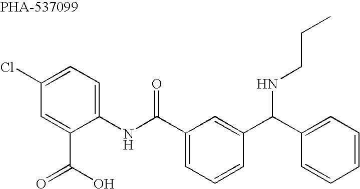 | 8 | 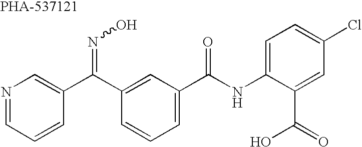 | 4 |
| 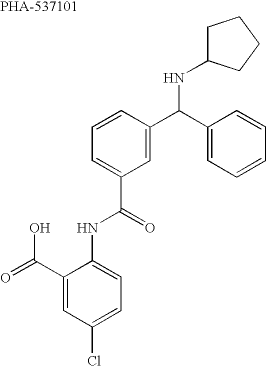 | 4 | 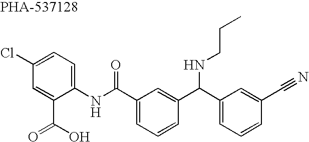 | 8 |
| 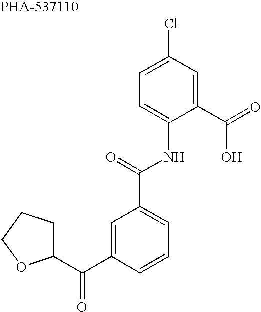 | 64 | 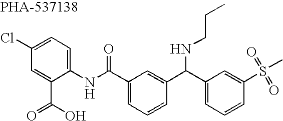 | 32 |
| 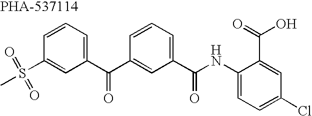 | 16 | 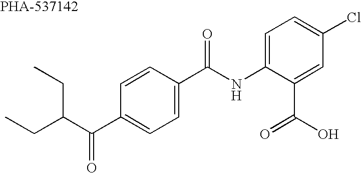 | 4 |
| 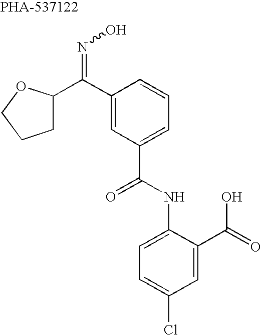 | 16 | 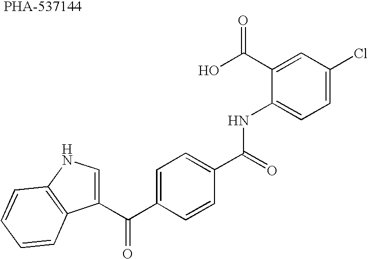 | 8 |
| 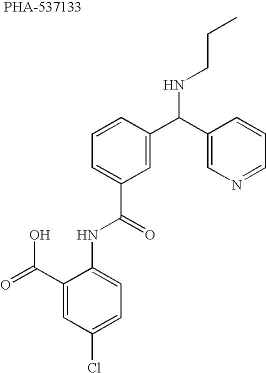 | 8 | 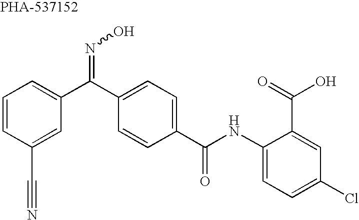 | 8 |
| 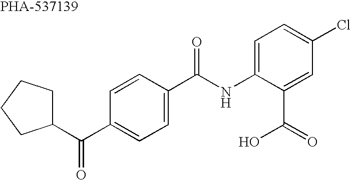 | 4 | 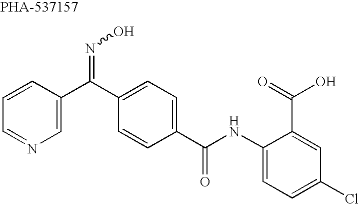 | 32 |
| 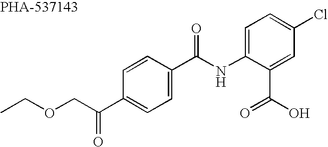 | 16 | 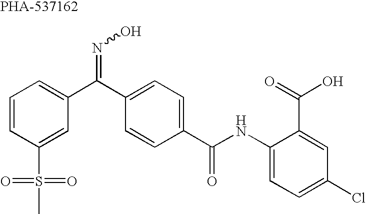 | 16 |
| 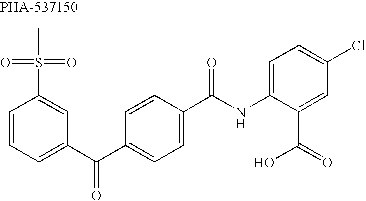 | 32 | 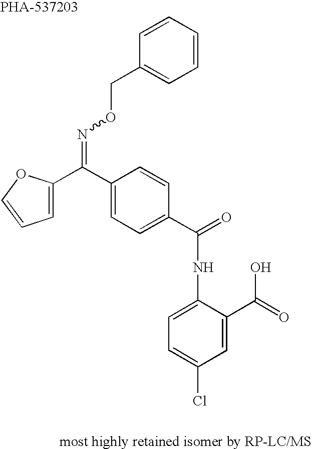 | 32 |

| | SA 9218 | | SA 9218 |
|---|---|---|---|
| Compound No., Structure | MIC | Compound No., Structure | MIC |
| 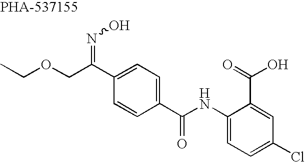 | 64 | 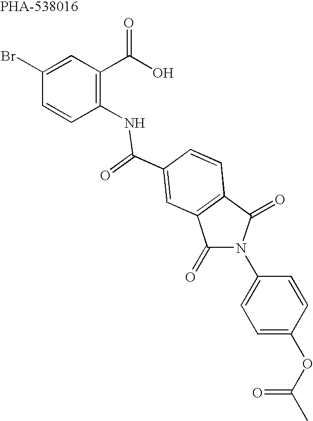 | 64 |
| 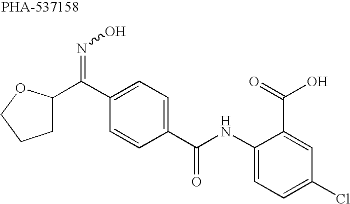 | 32 | 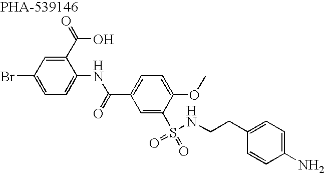 | 128 |
| 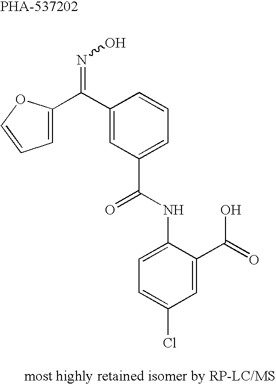 | 8 | 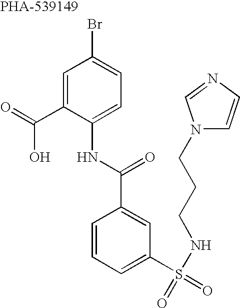 | 64 |
| 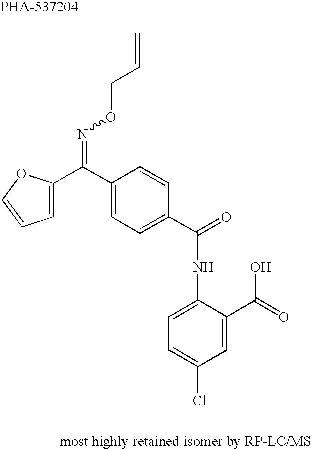 | 64 | 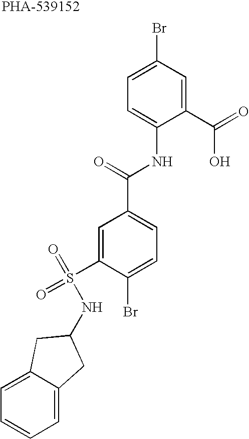 | 64 |
| 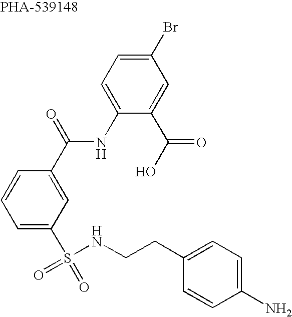 | 64 | 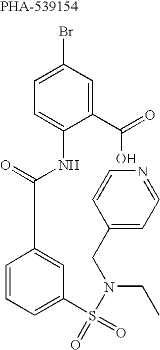 | 32 |
| 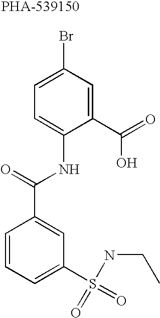 | 64 | 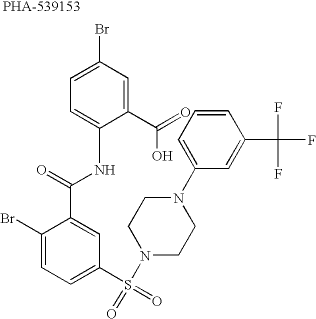 | 8 |
| 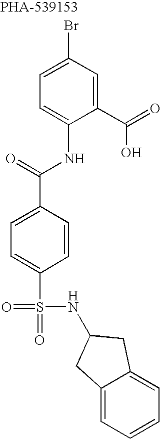 | 32 | 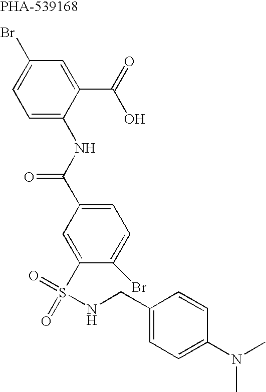 | 64 |
| 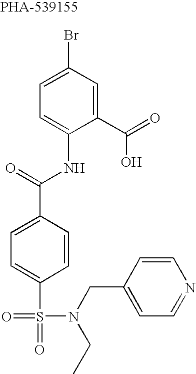 | 32 | 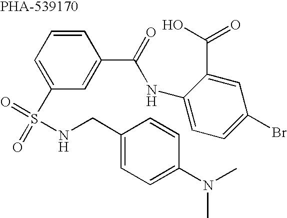 | 64 |
| 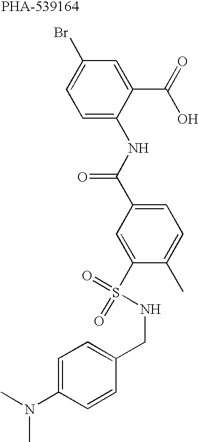 | 128 | 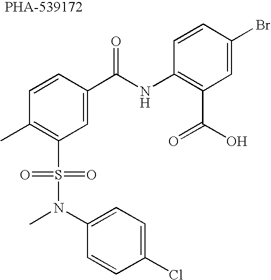 | 1 |
| 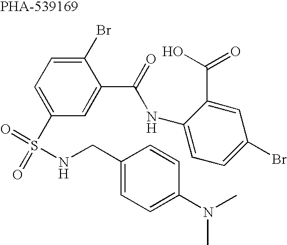 | 16 | 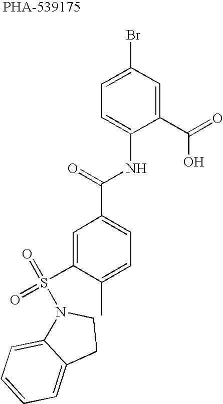 | 1 |
| 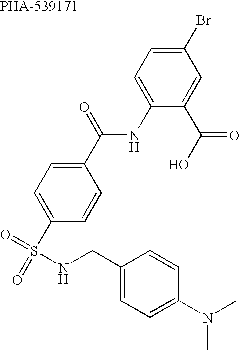 | 128 | 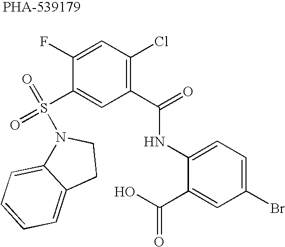 | 8 |
| 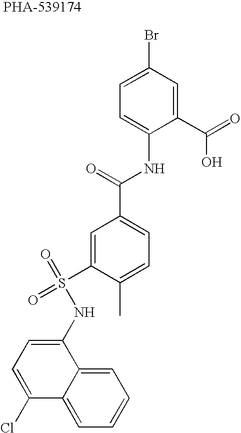 | 8 | 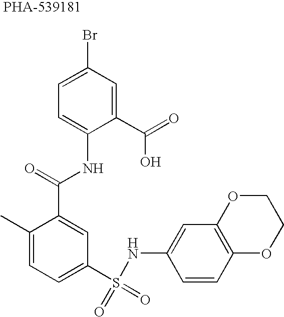 | 64 |
| 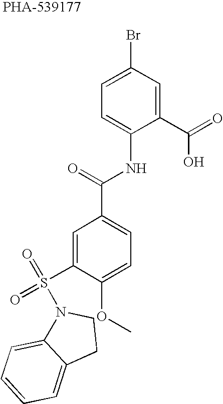 | 4 | 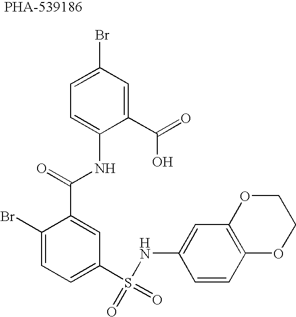 | 0.5 |
| 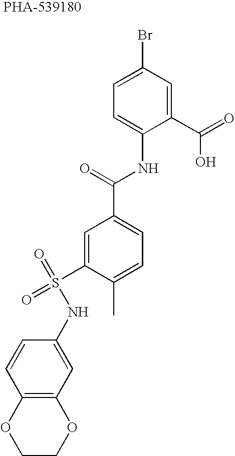 | 1 | 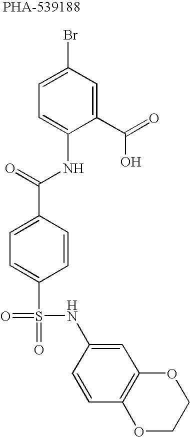 | 16 |
| 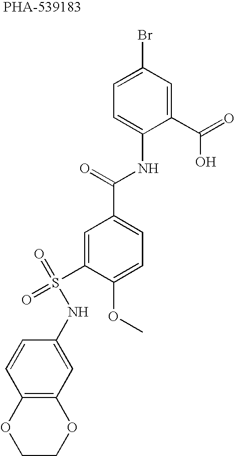 | 8 | 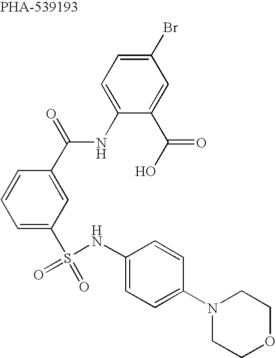 | 32 |
| 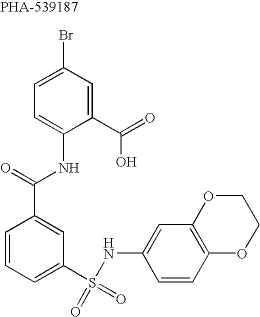 | 4 | 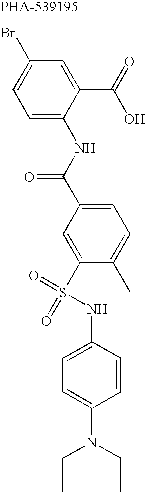 | 64 |
| 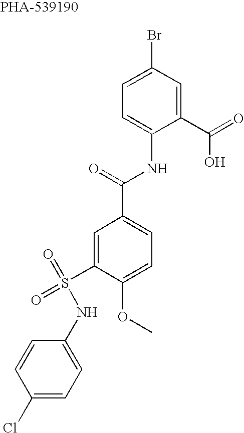 | 16 | 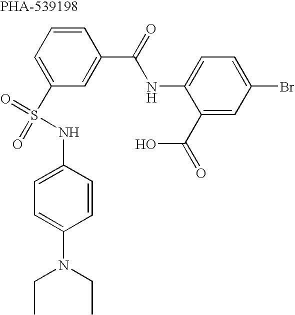 | 128 |
| 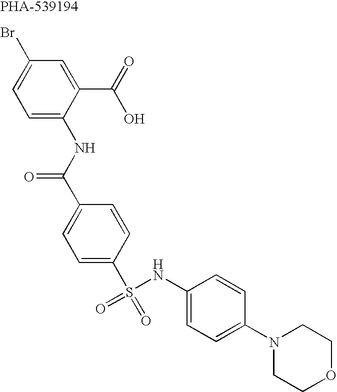 | 64 | 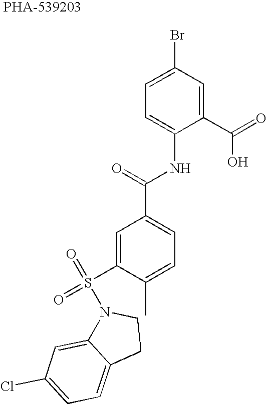 | 1 |
| 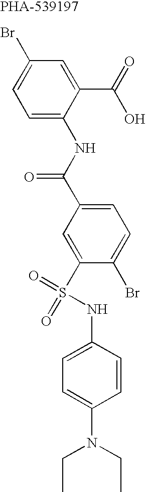 | 64 | 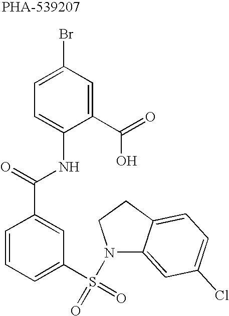 | 2 |
| 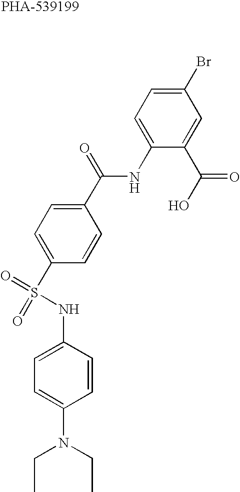 | 32 | 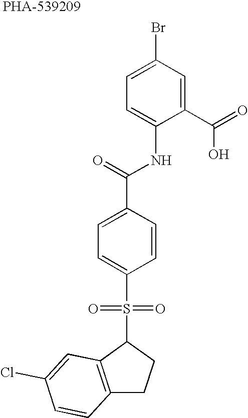 | 0.5 |
| 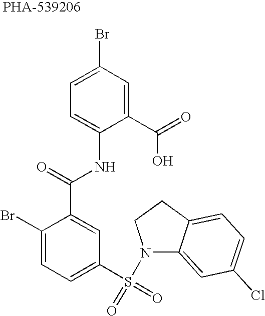 | 2 | 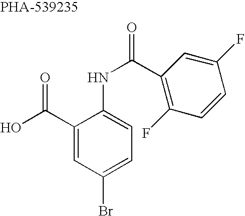 | 128 |
| 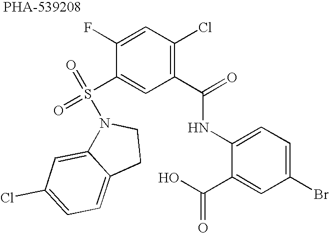 | 16 | 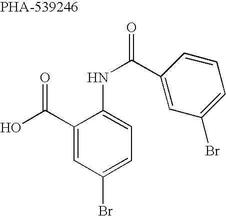 | 8 |
| 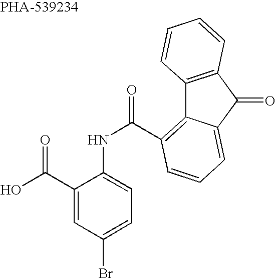 | 128 | 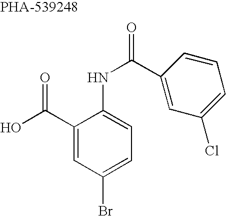 | 8 |
| 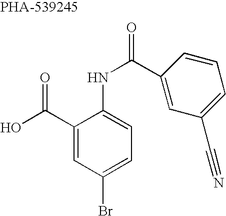 | >128 | 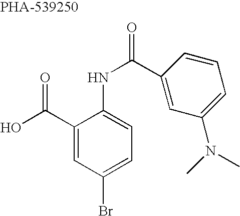 | 32 |
| 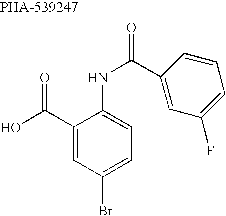 | 64 | 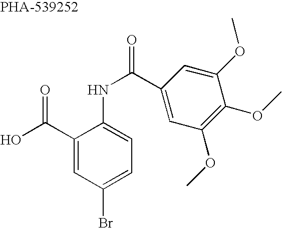 | 32 |
| 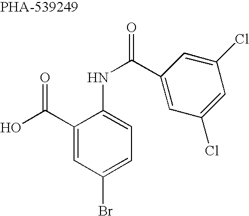 | 8 | 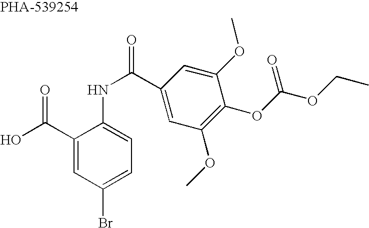 | 128 |
| 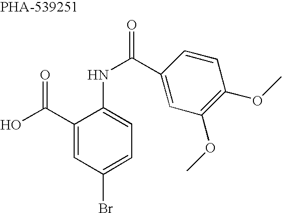 | 128 | 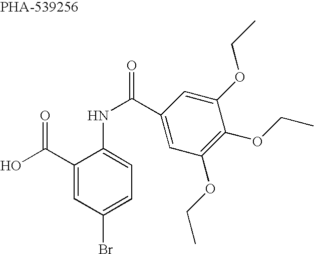 | 32 |
| 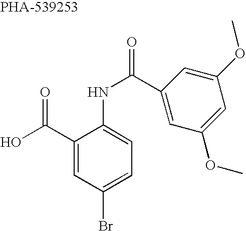 | 16 | 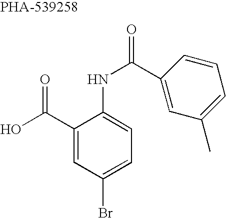 | 64 |
| 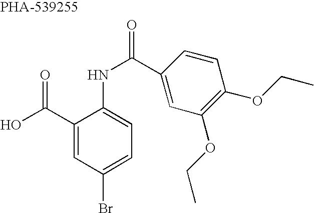 | >128 | 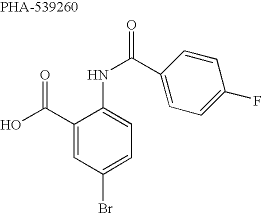 | 64 |
| 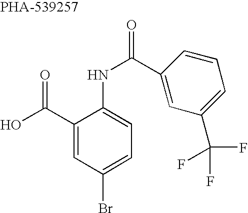 | 8 | 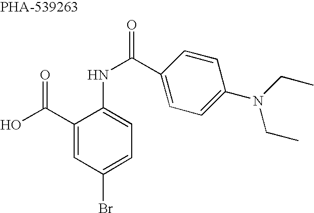 | 32 |
| 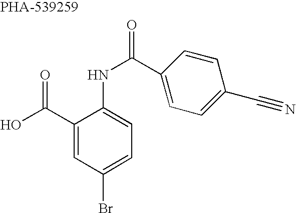 | 128 | 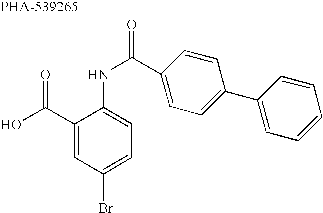 | 32 |
| 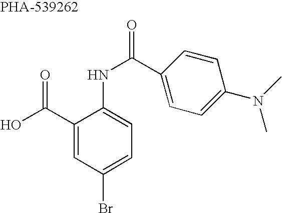 | 32 | 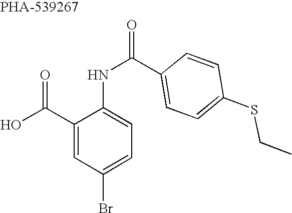 | 0.5 |
| 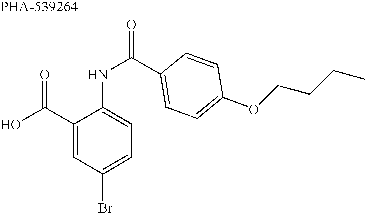 | 8 | 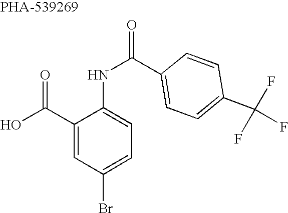 | 32 |
| 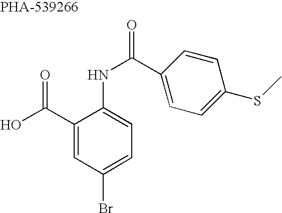 | 2 | 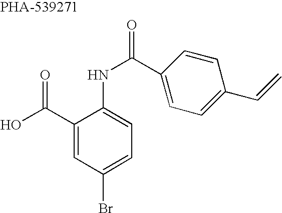 | >128 |
| 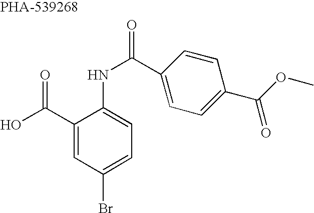 | 32 | 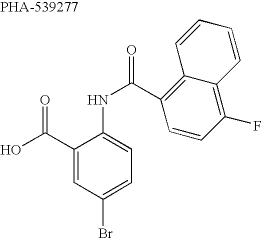 | 32 |
| 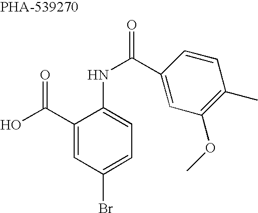 | >128 | 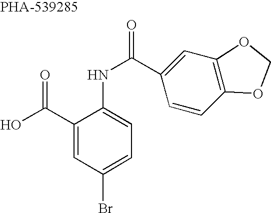 | 16 |
| 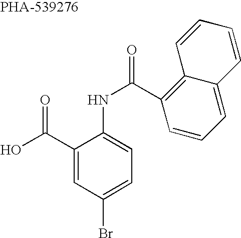 | 32 | 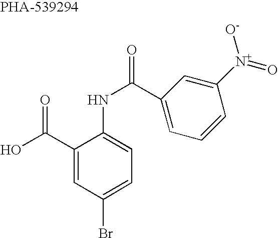 | 128 |
| 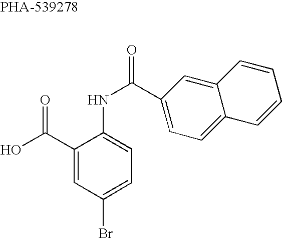 | 2 | 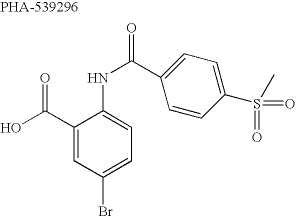 | 64 |
| 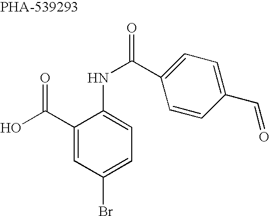 | >128 | 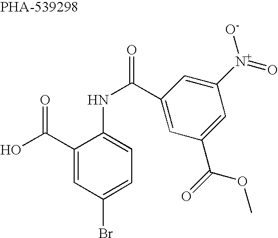 | 64 |
| 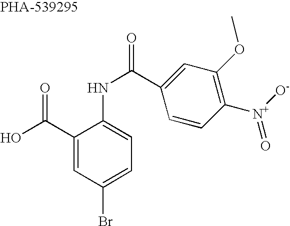 | 32 | 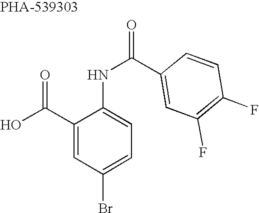 | 32 |
| 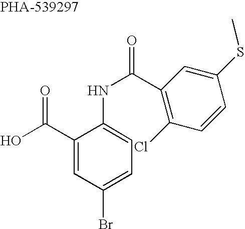 | >128 | 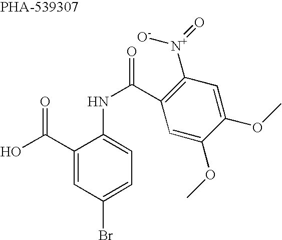 | >128 |
| 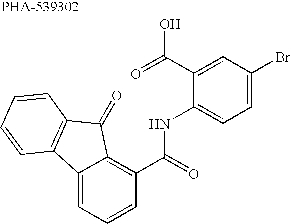 | >128 | 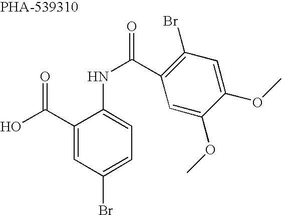 | >128 |
| 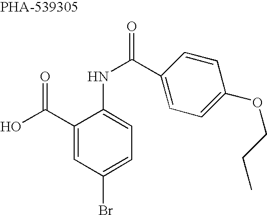 | 64 | 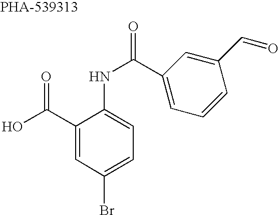 | >128 |
| 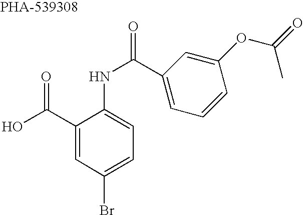 | 128 | 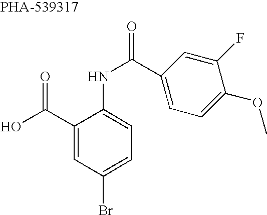 | 16 |
| 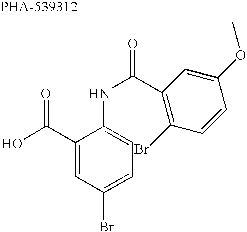 | 128 | 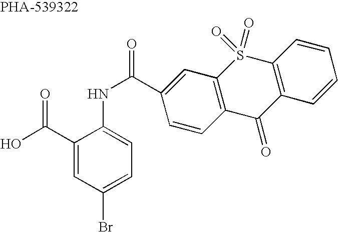 | 16 |
| 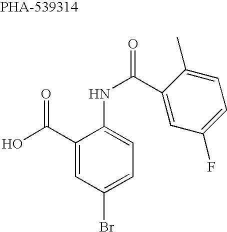 | 64 | 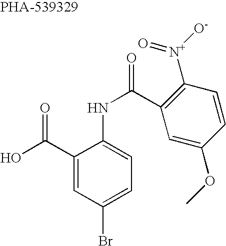 | >128 |
| 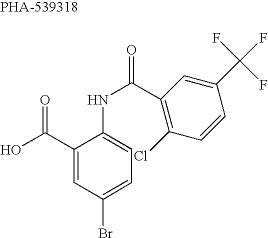 | >128 | 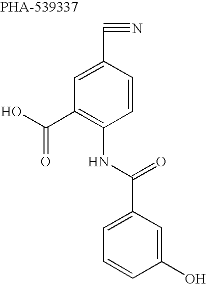 | 32 |
| 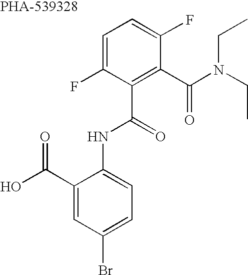 | >128 | 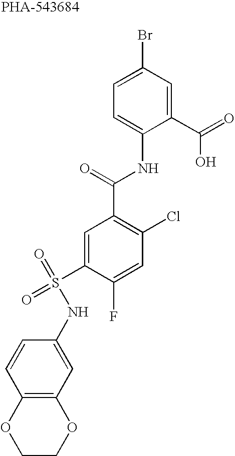 | 128 |
| 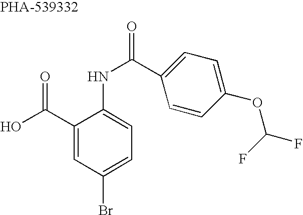 | 64 | 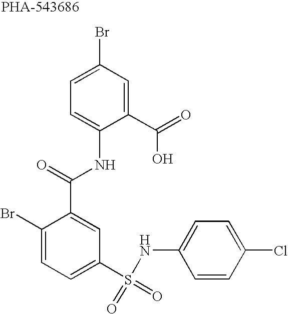 | 4 |
| 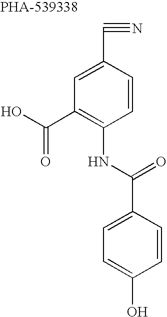 | 64 | 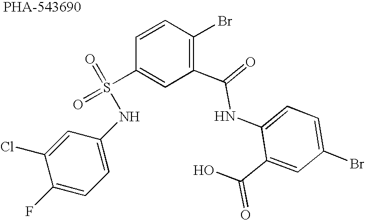 | 32 |
| 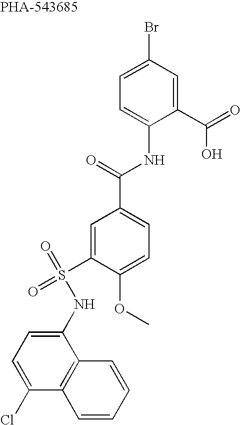 | >128 | 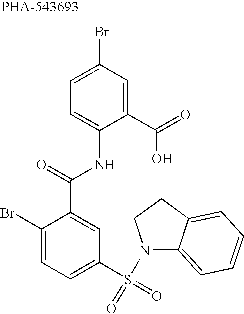 | 2 |
| 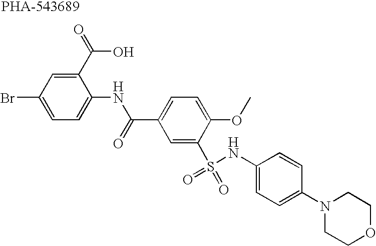 | 64 | 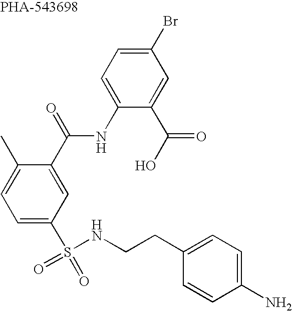 | >128 |
| 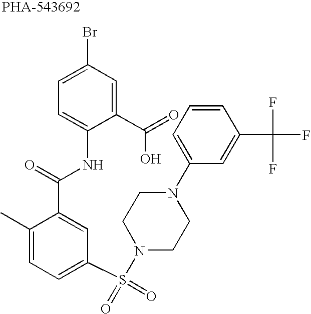 | 16 | 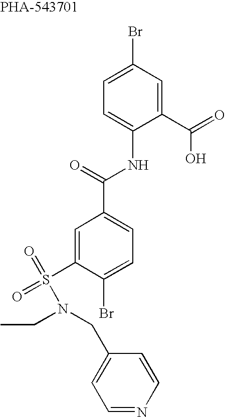 | 128 |
| 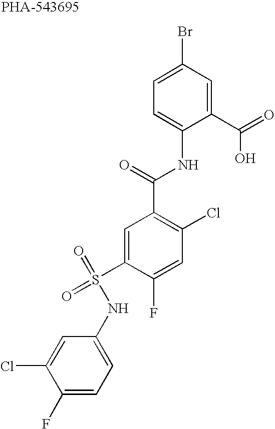 | >128 | 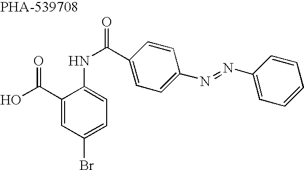 | 16 |
| 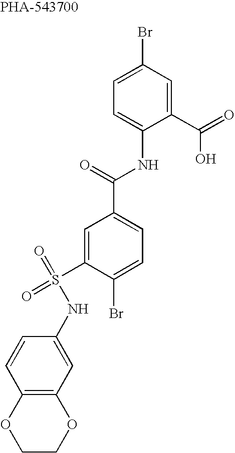 | 64 | 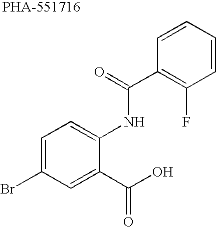 | 128 |
| 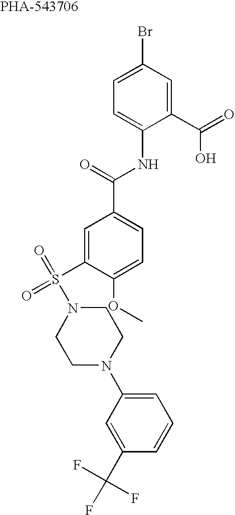 | 32 | 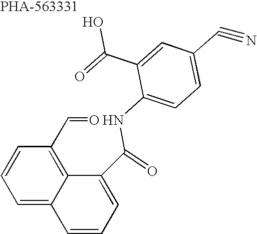 | >128 |
| 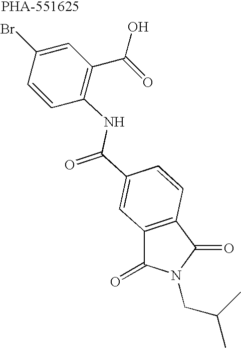 | 2 | 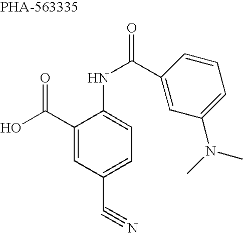 | 8 |
| 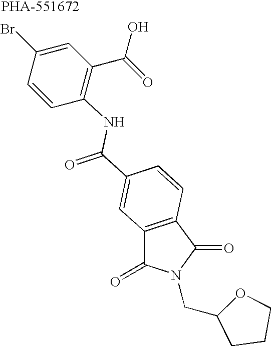 | 8 | 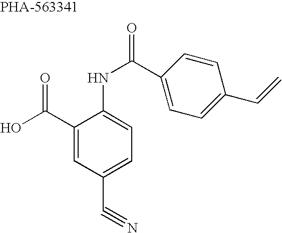 | 8 |
| 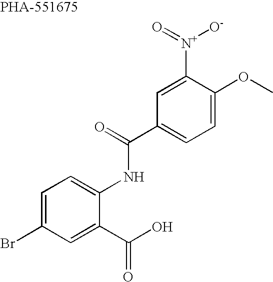 | 32 | 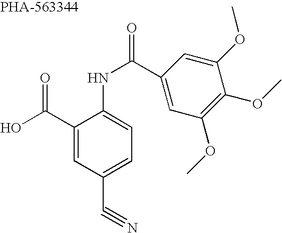 | 64 |
| 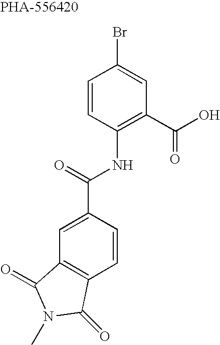 | 128 | 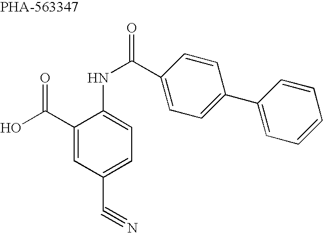 | 64 |
| 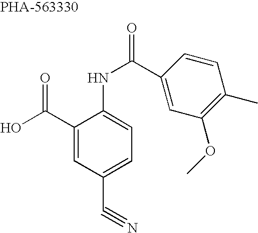 | >128 | 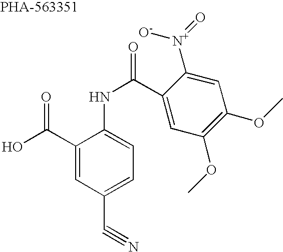 | >128 |
| 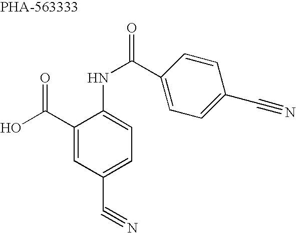 | >128 | 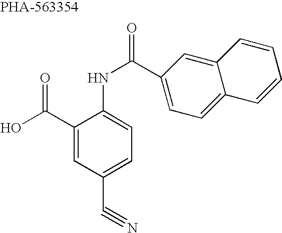 | 2 |
| 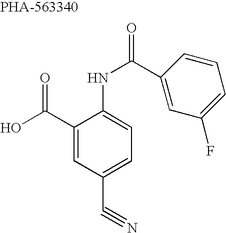 | 64 | 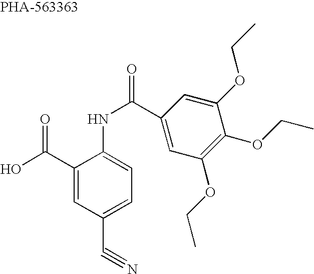 | 16 |
| 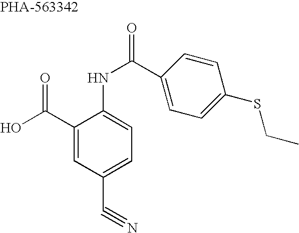 | 2 | 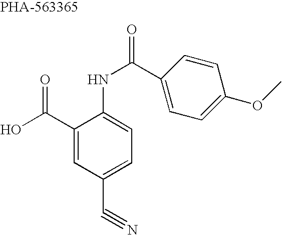 | 16 |
| 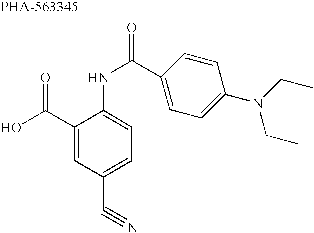 | 16 | 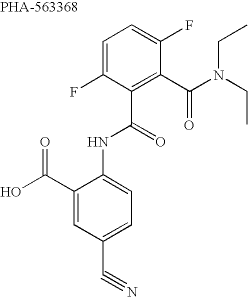 | >128 |
| 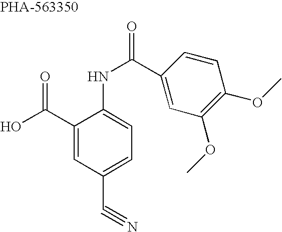 | 64 | 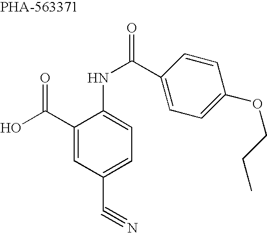 | 16 |
| 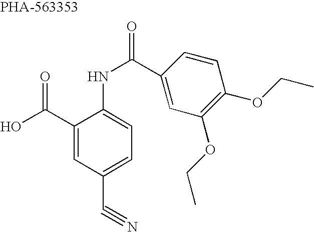 | 128 | 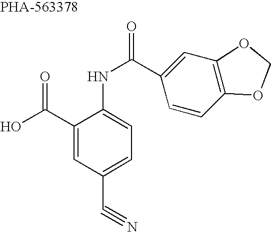 | 16 |
| 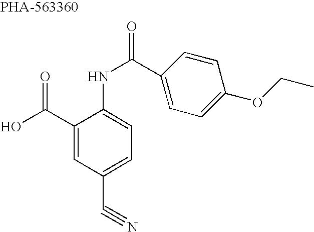 | 32 | 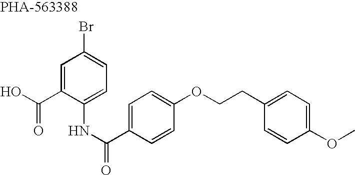 | >128 |
| 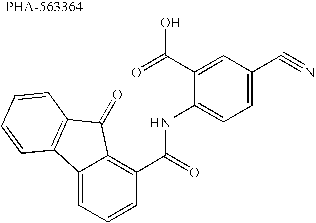 | >128 | 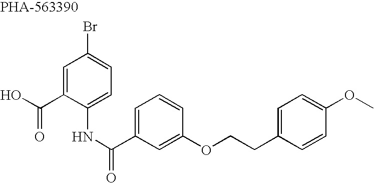 | 32 |
| 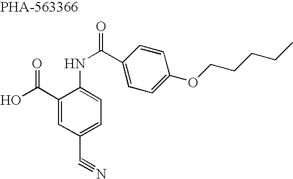 | 4 | 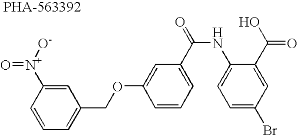 | 16 |
| 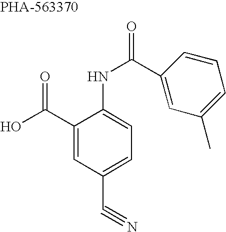 | 32 | 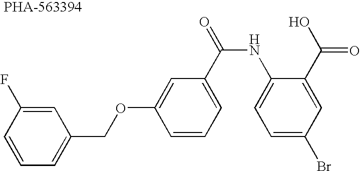 | 16 |
| 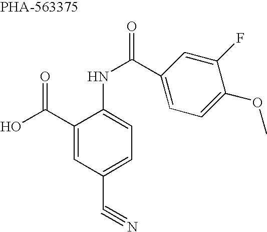 | 8 | 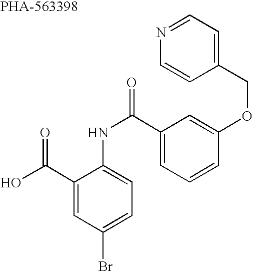 | >128 |
| 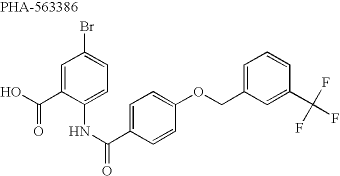 | 32 | 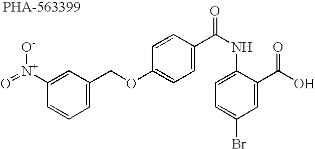 | 16 |
| 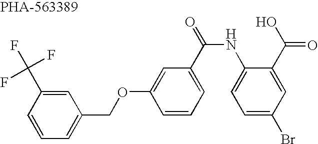 | 64 | 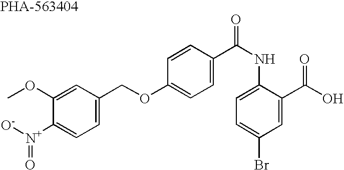 | 8 |
| 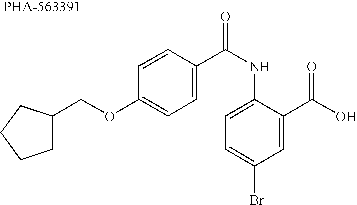 | >128 | 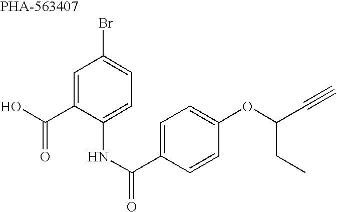 | >128 |
| 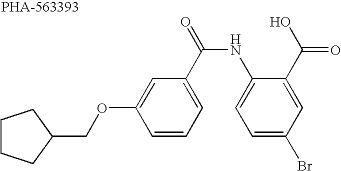 | 128 | 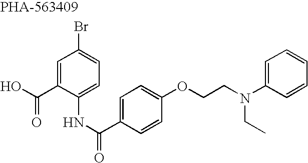 | 64 |
| 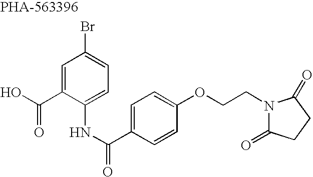 | >128 | 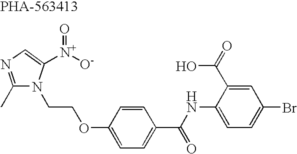 | 128 |
| 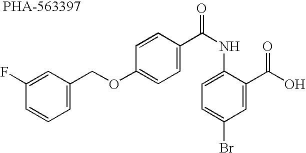 | 32 | 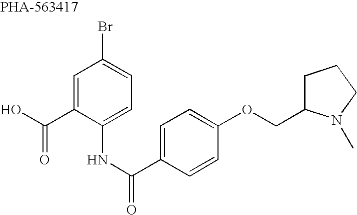 | >128 |
| 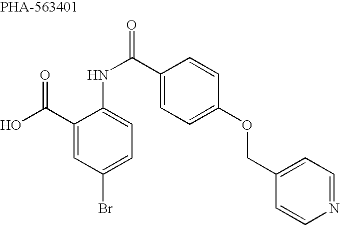 | >128 | 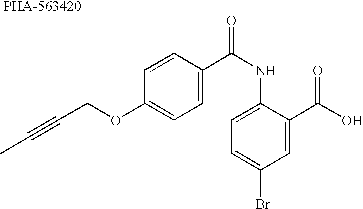 | 16 |
| 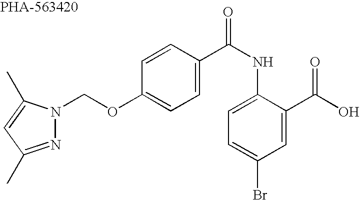 | 64 | 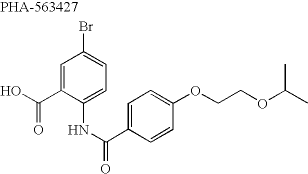 | >128 |
| 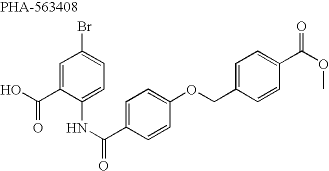 | >128 | 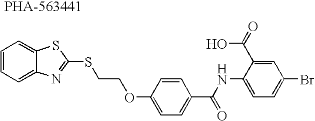 | 64 |
| 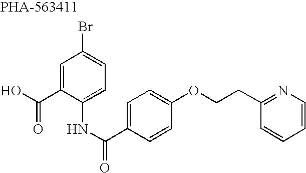 | 128 | 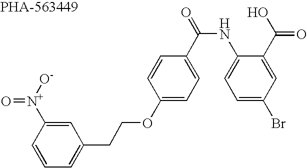 | 64 |
| 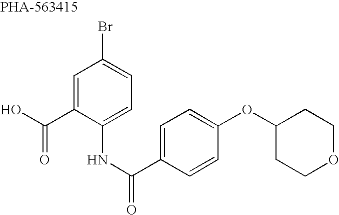 | 128 | 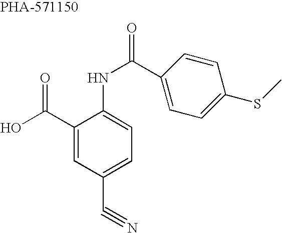 | 0.5 |
| 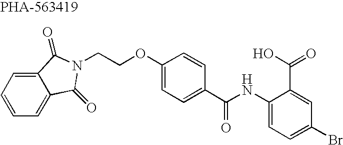 | 64 | 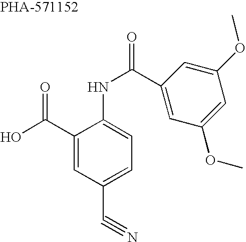 | 8 |
| 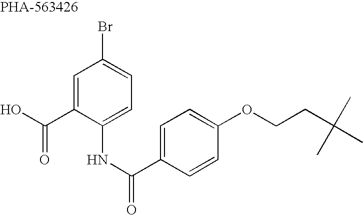 | 64 | 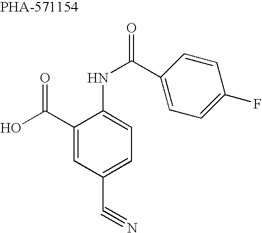 | 128 |
| 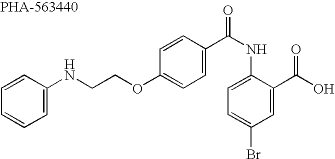 | 64 | 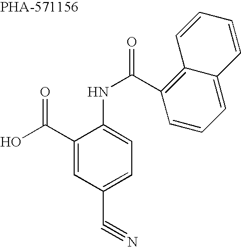 | 16 |
| 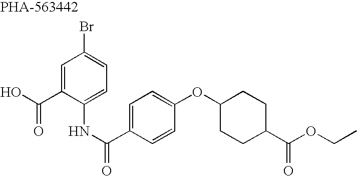 | >128 | 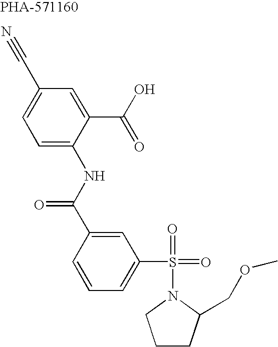 | 64 |
| 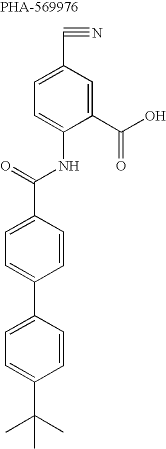 | 32 | 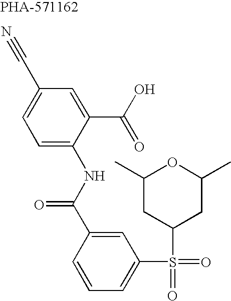 | 16 |

| | SA 9218 |
|---|---|
| Compound No., Structure | MIC |
| PHA-571151 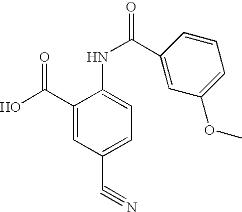 | 8 |
| PHA-571153 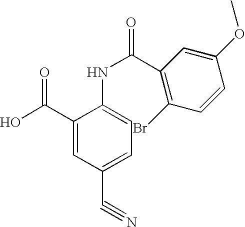 | 64 |
| PHA-571155 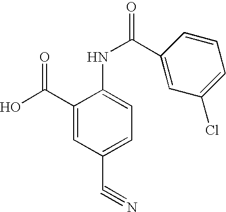 | 32 |
| PHA-571157 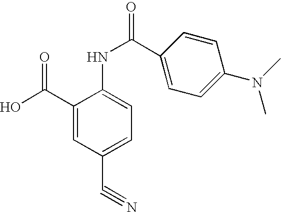 | 32 |
| PHA-571161 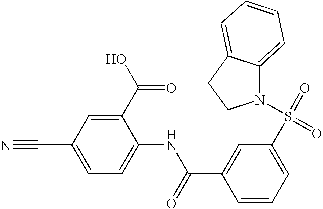 | >128 |
| PHA-571164 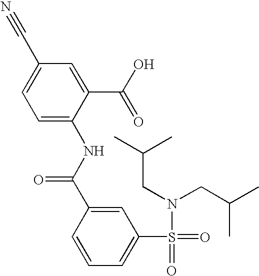 | 8 |
| PHA-571169 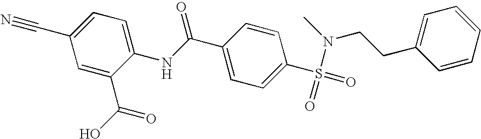 | 32 |
| PHA-571172 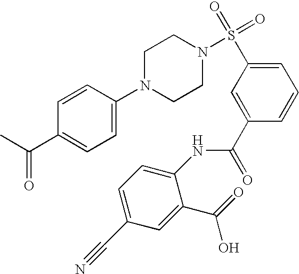 | 32 |
| PHA-571176 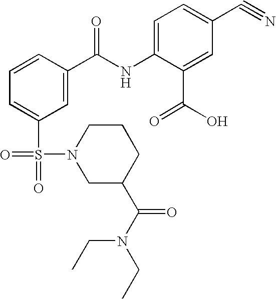 | 64 |
| PHA-571183 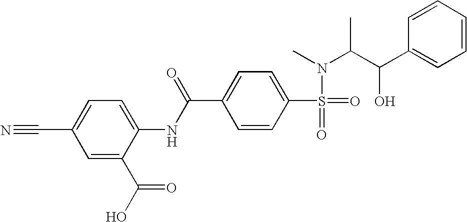 | 32 |
| PHA-571188 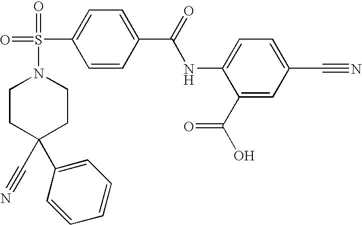 | 8 |
| PHA-571194 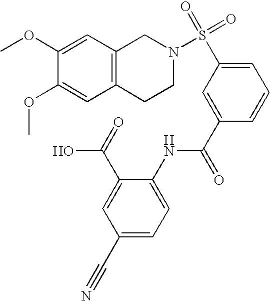 | 4 |
| PHA-571197 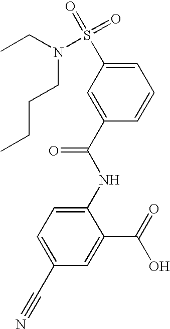 | 16 |
| PHA-571199 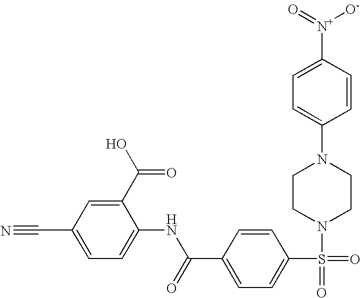 | 64 |
| PHA-571203 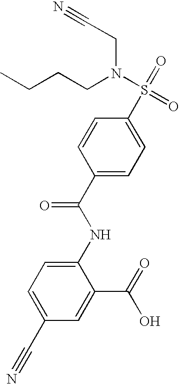 | 32 |
| PHA-571207 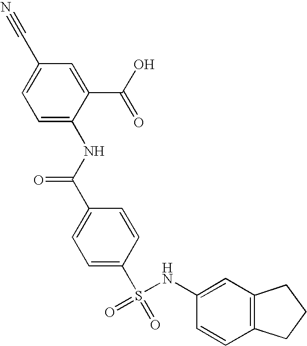 | 32 |
| PHA-571214 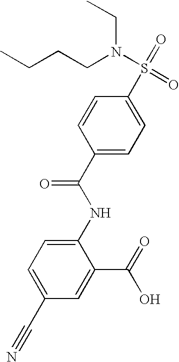 | 16 |
| PHA-571216 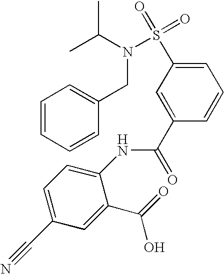 | 32 |
| PHA-571224 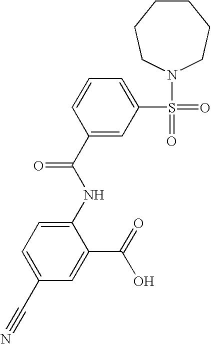 | 8 |
| PHA-571228 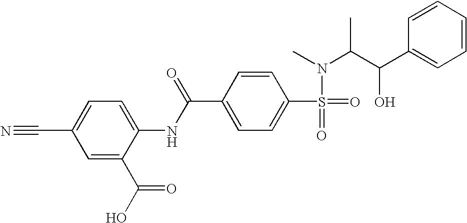 | 32 |
| PHA-571231 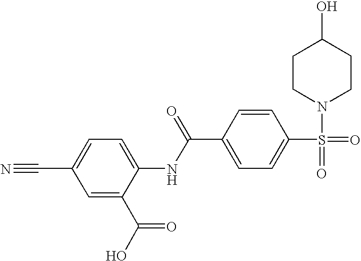 | >128 |
| PHA-571234 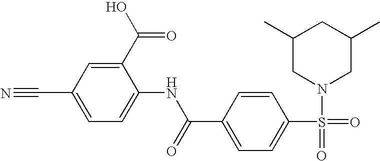 | 8 |
| PHA-571237 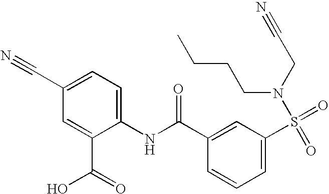 | 16 |
| PHA-571239 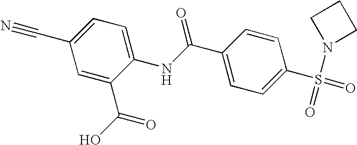 | 128 |
| PHA-571241 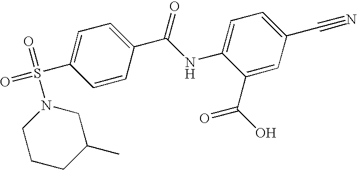 | 16 |
| PHA-571243 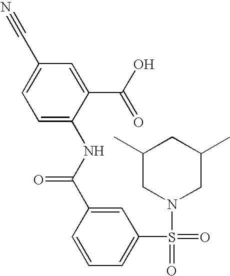 | 4 |
| PHA-571249 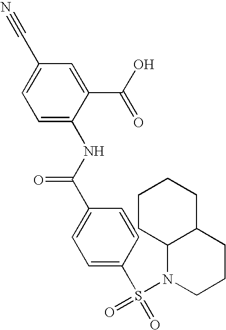 | 16 |
| PHA-571255 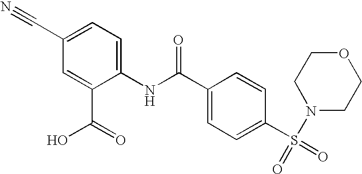 | >128 |
| PHA-571258 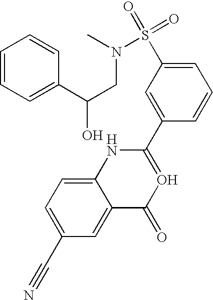 | 8 |
| PHA-571262 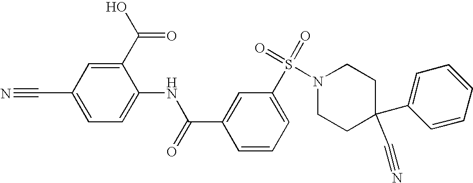 | 32 |
| PHA-571264 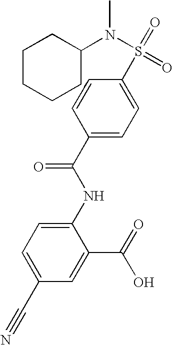 | 32 |
| PHA-571267 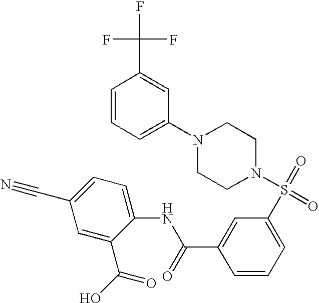 | 32 |
| PHA-571270 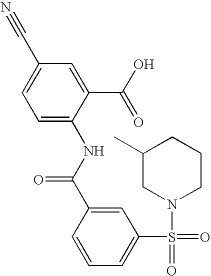 | 8 |
| PHA-571272 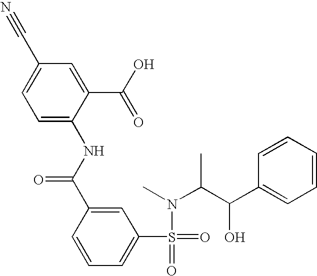 | 32 |
| PHA-571280 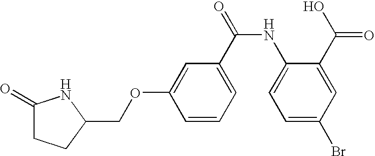 | >128 |
| PHA-571282 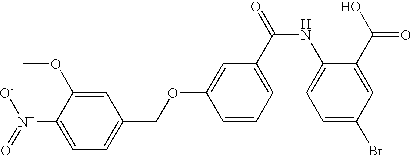 | 16 |
| PHA-571285 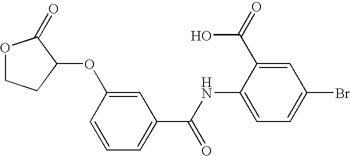 | 64 |
| PHA-571289 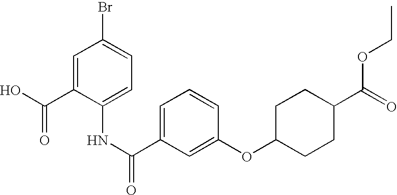 | 32 |
| PHA-610940 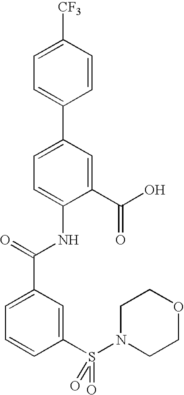 | >128 |
| PHA-610942 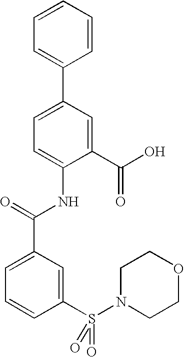 | >128 |
| PHA-656807 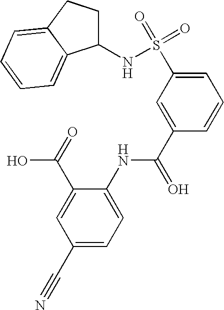 | 64 |
| PHA-656809 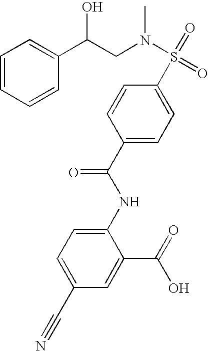 | 64 |
| PHA-656811 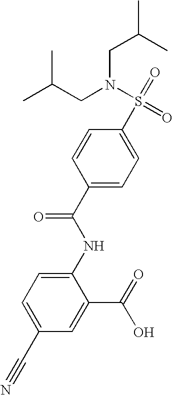 | 32 |
| PHA-656859 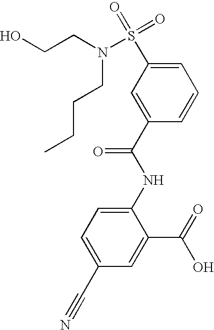 | 16 |
| PHA-656861 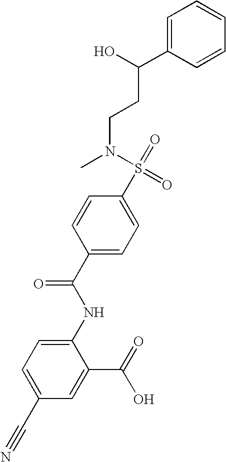 | 32 |
| PHA-656863 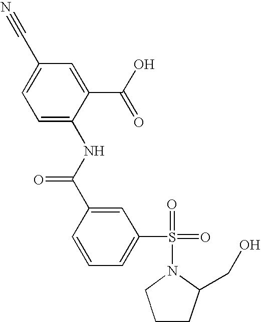 | 8 |
| PHA-656867 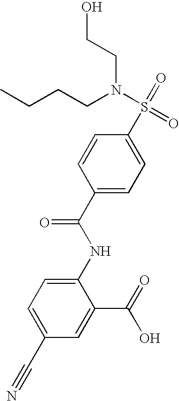 | 64 |
| PHA-656870 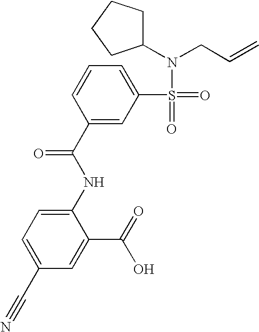 | 8 |
| PHA-656872 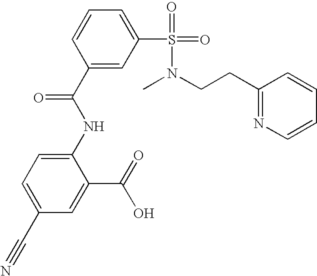 | >128 |
| PHA-656882 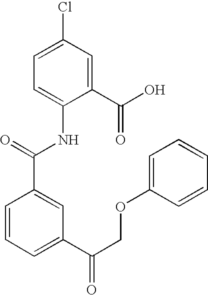 | 16 |
| PHA-656884 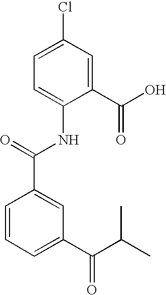 | 16 |
| PHA-656886 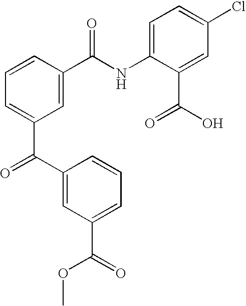 | 16 |
| PHA-656888 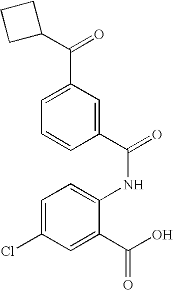 | 16 |
| PHA-656890 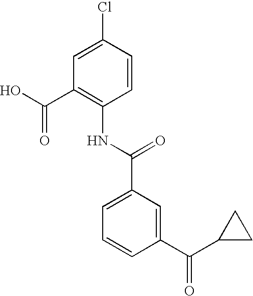 | 16 |
| PHA-656892 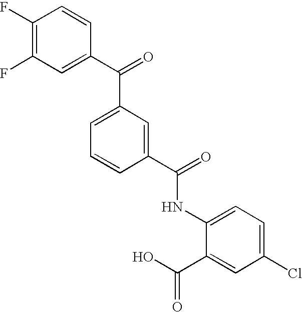 | 8 |
| PHA-656894 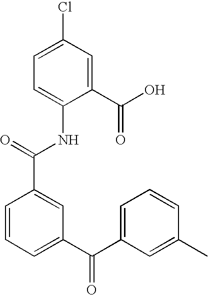 | 16 |
| PHA-662254 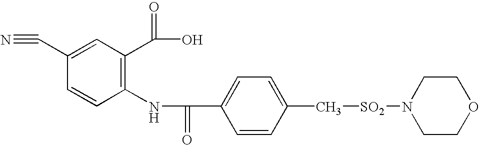 | >128 |
| PHA-679756 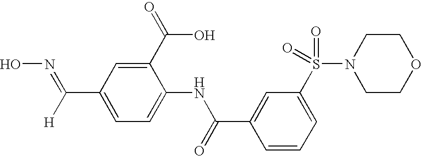 | >128 |
| PHA-687570 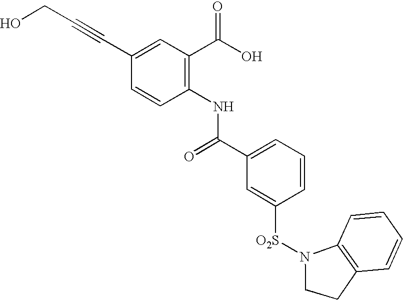 | 128 |
| PHA-708979 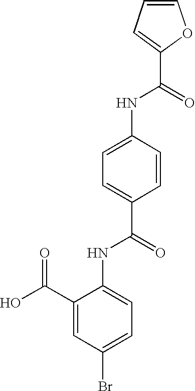 | >128 |
| PHA-713389 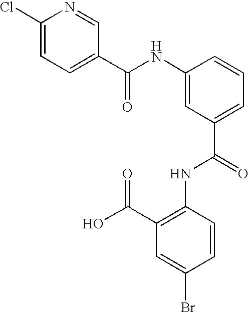 | >128 |
| PHA-713391 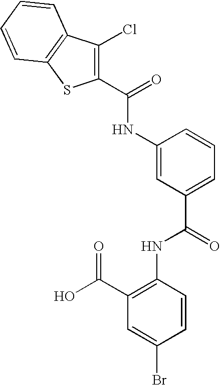 | >128 |
| PHA-713393 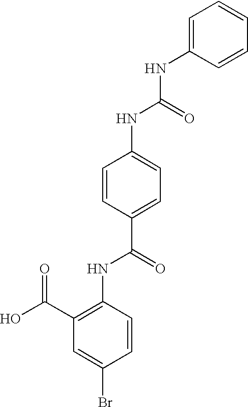 | >128 |
| PHA-713397 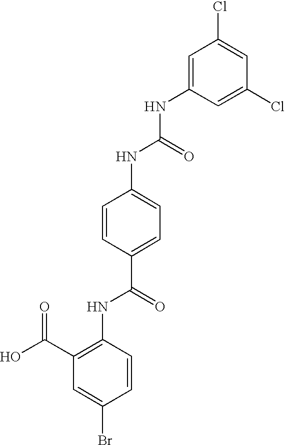 | >128 |
| PHA-738532 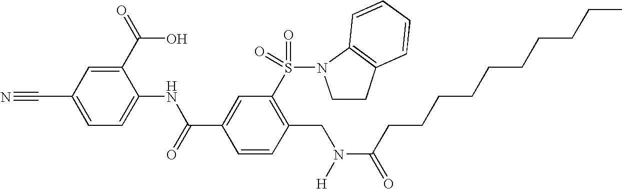 | 32 |
| PHA-748361 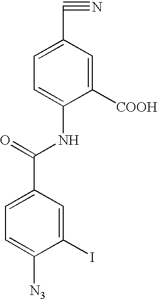 | 8 |
| PNU-276672 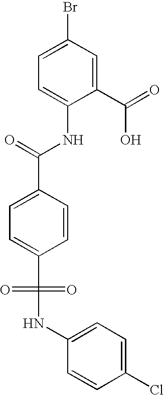 | |
| PNU-292577 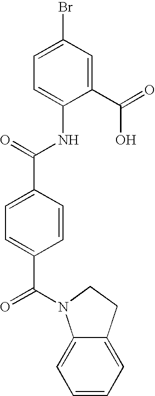 | 128 |
| PHA-571167 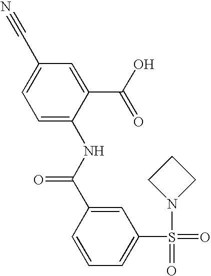 | 32 |
| PHA-571170 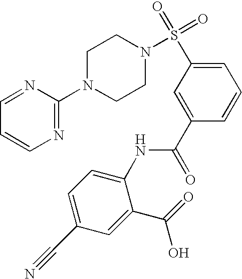 | 64 |
| PHA-571174 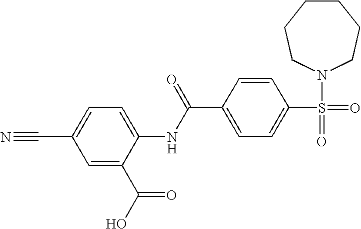 | 64 |
| PHA-571182 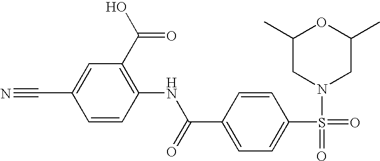 | 64 |
| PHA-571186 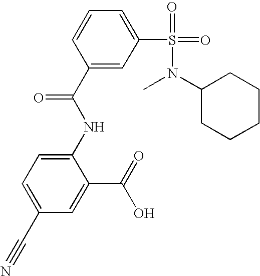 | 128 |
| PHA-571189 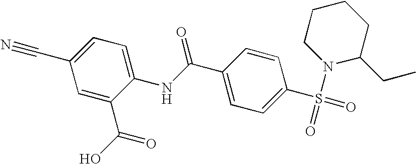 | 64 |
| PHA-571196 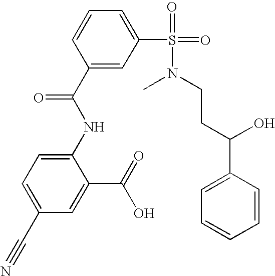 | 64 |
| PHA-571198 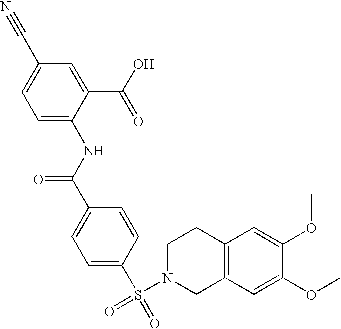 | >128 |
| PHA-571202 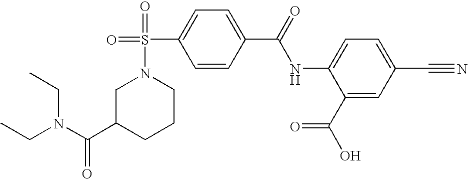 | 128 |
| PHA-571205 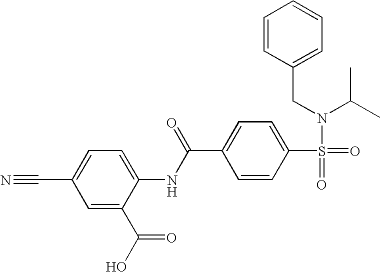 | 32 |
| PHA-571208 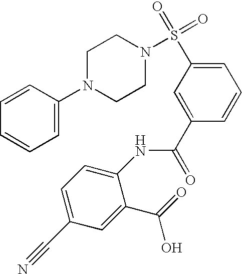 | 64 |
| PHA-571215 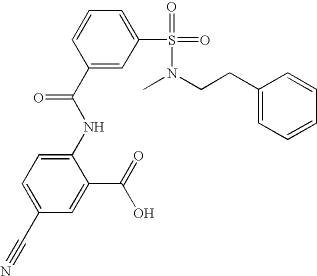 | 8 |
| PHA-571219 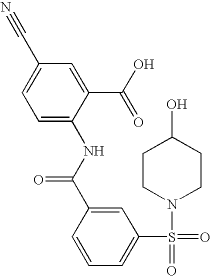 | 32 |
| PHA-571226 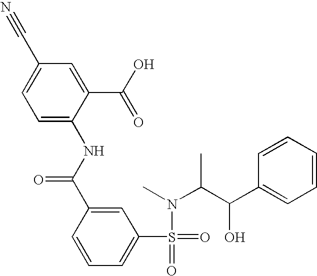 | 64 |
| PHA-571230 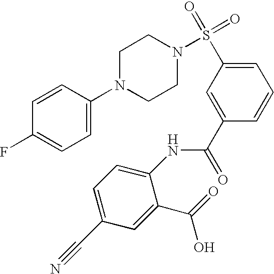 | 16 |
| PHA-571232 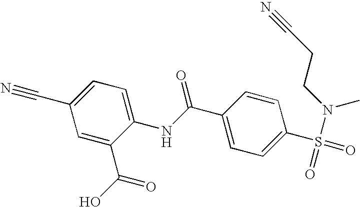 | >128 |
| PHA-571235 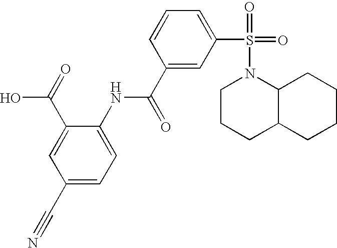 | 8 |
| PHA-571238 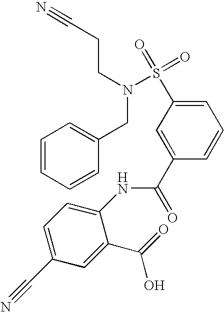 | 128 |
| PHA-571240 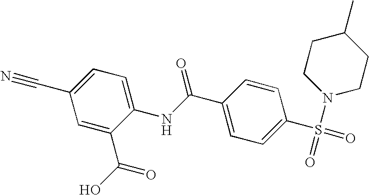 | 16 |
| PHA-571242 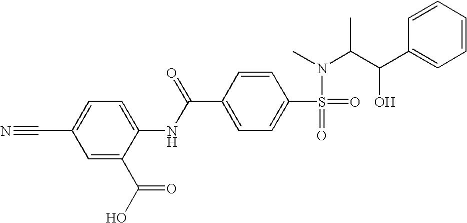 | 32 |
| PHA-571246 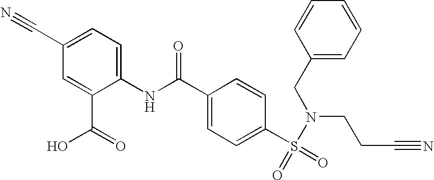 | 32 |
| PHA-571253 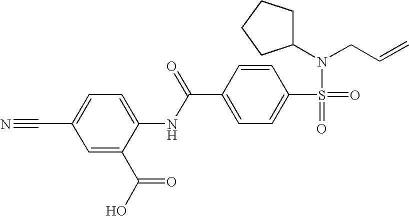 | 16 |
| PHA-571257 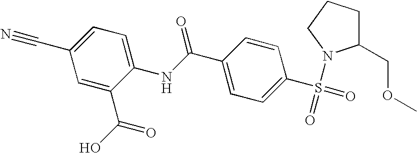 | 64 |
| PHA-571260 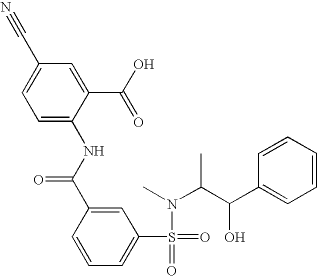 | 32 |
| PHA-571263 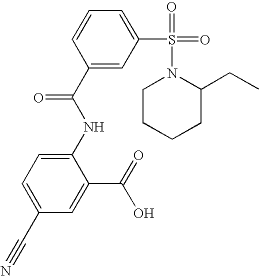 | 16 |
| PHA-571265 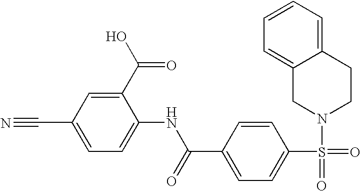 | 16 |
| PHA-571269 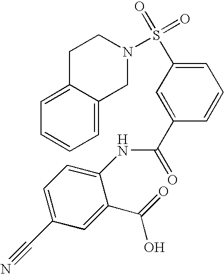 | 16 |
| PHA-571271 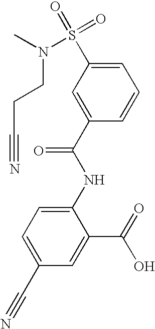 | 64 |
| PHA-571273 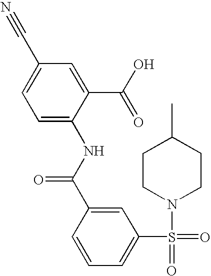 | 8 |
| PHA-571281 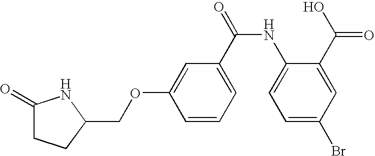 | 128 |
| PHA-571283 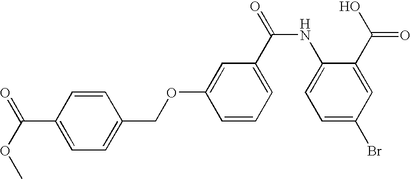 | 16 |
| PHA-571287 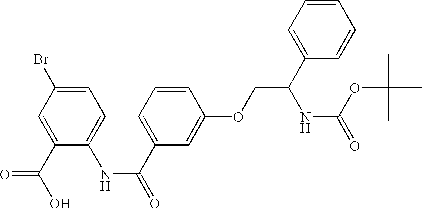 | 2 |
| PHA-571292 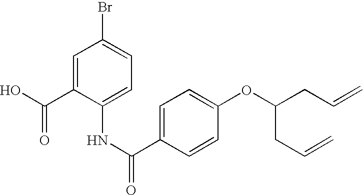 | 32 |
| PHA-610941 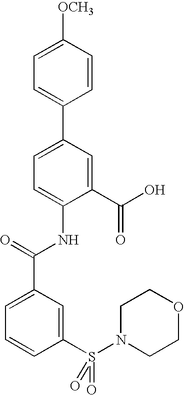 | >128 |
| PHA-630426 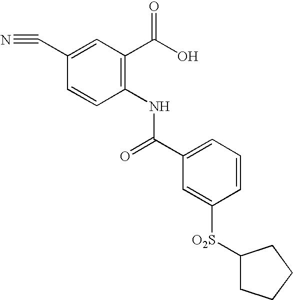 | >128 |
| PHA-656808 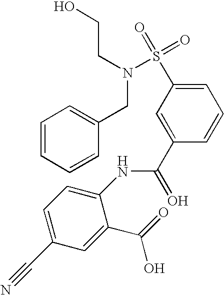 | 64 |
| PHA-656810 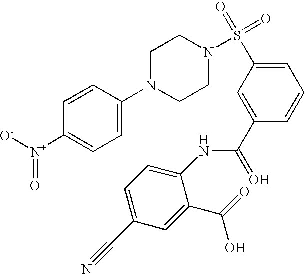 | 2 |
| PHA-656820 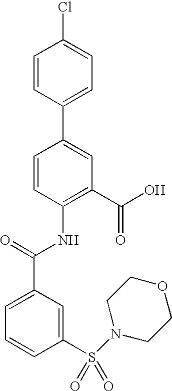 | >128 |
| PHA-656860 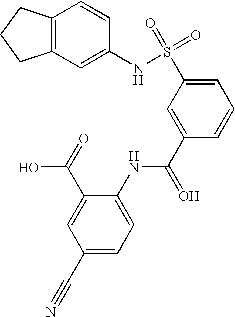 | 8 |
| PHA-656862 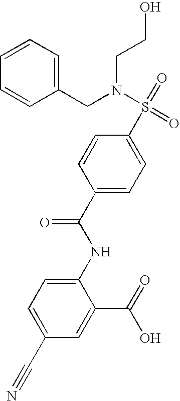 | 32 |
| PHA-656866 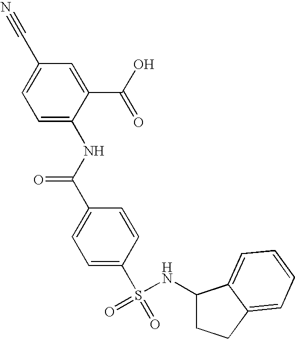 | >128 |
| PHA-656868 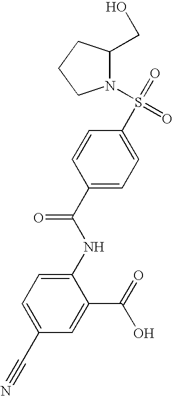 | >128 |
| PHA-656871 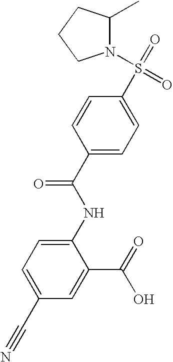 | 128 |
| PHA-656880 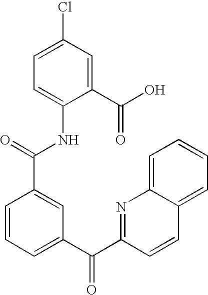 | 16 |
| PHA-656883 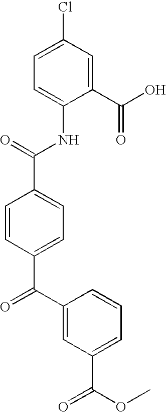 | 16 |
| PHA-656885 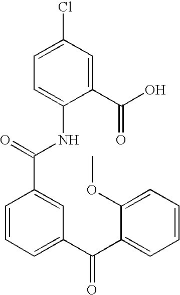 | 16 |
| PHA-656887 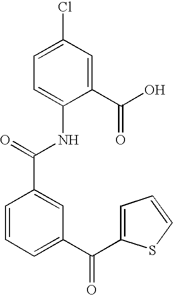 | 8 |
| PHA-656889 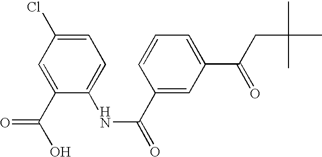 | 16 |
| PHA-656891 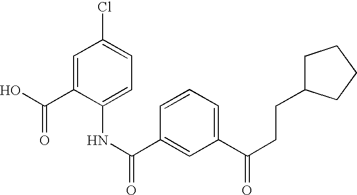 | 16 |
| PHA-656893 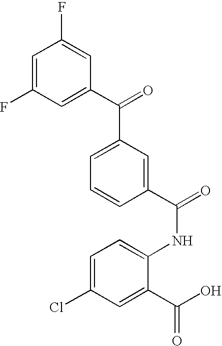 | 8 |
| PHA-662253 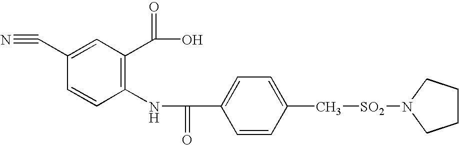 | 128 |
| PHA-662412 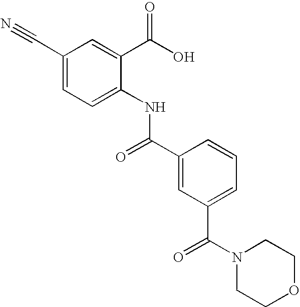 | 64 |
| PHA-679759 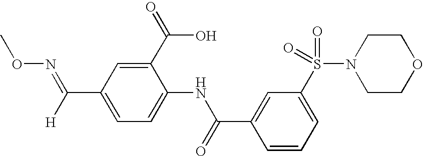 | >128 |
| PHA-708922 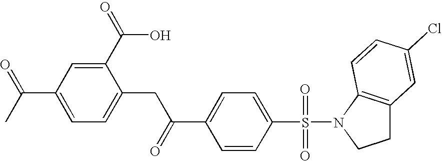 | >128 |
| PHA-708977 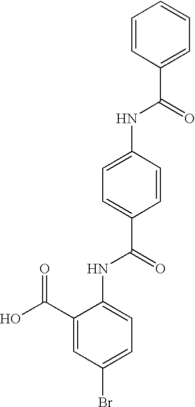 | >128 |
| PHA-708987 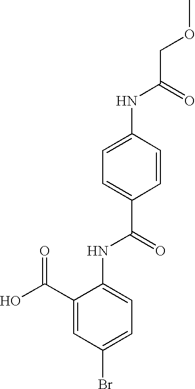 | >128 |
| PHA-713390 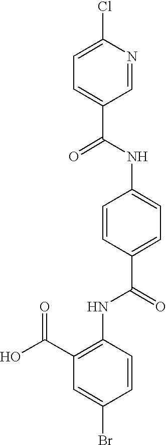 | >128 |
| PHA-713392 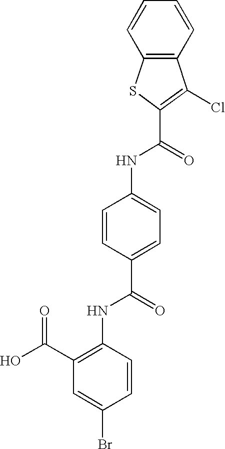 | >128 |
| PHA-713395 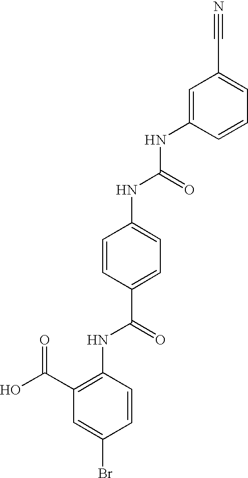 | >128 |
| PHA-738531 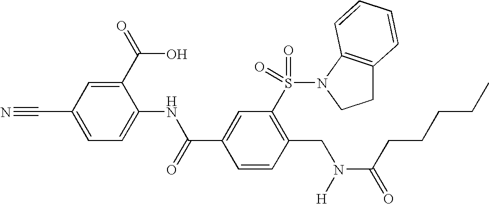 | 64 |
| PHA-740499 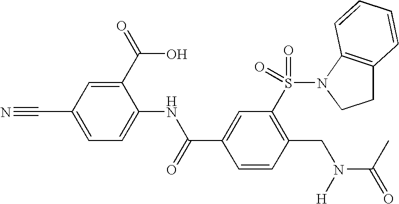 | 128 |
| PNU-276556 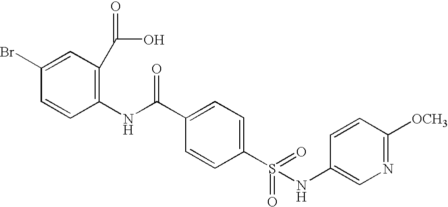 | |
| PNU-276873 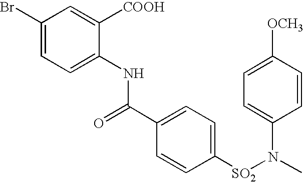 | |
| PNU-282858 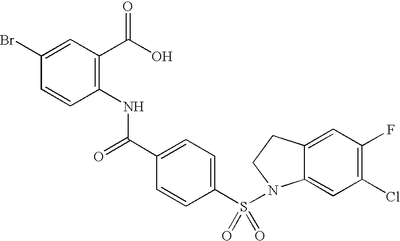 | |
| PNU-282860 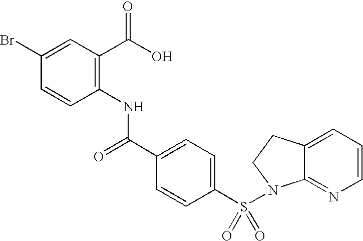 | |
| PNU-291997 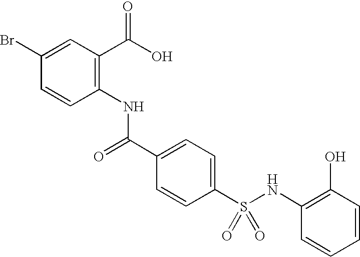 | 1 |
| PNU-281164 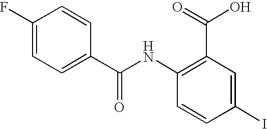 | >128 |
| PNU-282859 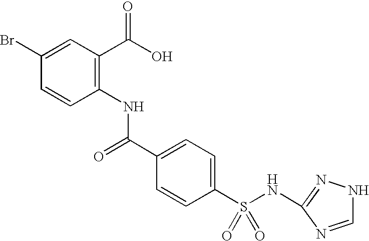 | 32 |
| PNU-290881A 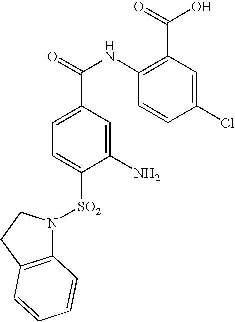 | 4 |

1. A compound of formula I, 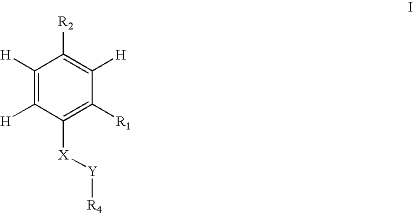
or a pharmaceutically acceptable salt thereof,
wherein
X═NH
Y═CO, CS, —C(═N═CN) or
X and Y together form an alkene, or C₃-C₅ cycloalkyl;
R₁ is —COOH;
R₂ is an electron withdrawing group;
R₄ is an optionally substituted aryl, provided that the aryl is not simultaneously substituted with a sulfonamide and a urea or thiourea, further provided that the aryl is not solely substituted at the ortho-position relative to Y, and still further provided that the aryl is not substituted with a group selected from 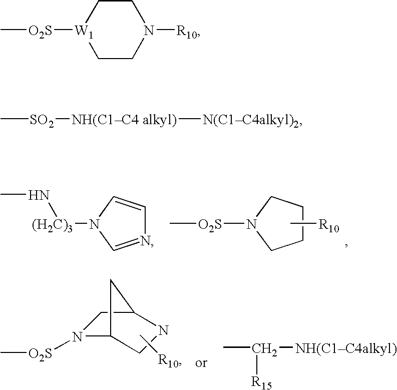
W₁ is N or CH;
R₁₀ is C₁-C₄ alkyl, C₁-C₄ substituted alkyl, Het, substituted Het, aryl, or substituted aryl; and
R₁₅ is H, C₁-C₄ alkyl, C₁-C₄ substituted alkyl, Het, substituted Het, C₄-C₇ cycloalkyl.

2. The compound of claim 1 having the formula 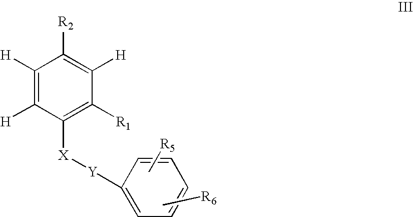
or a pharmaceutically acceptable salt thereof,
wherein
X═NH
Y═CO, CS, —C(═N—CN) or
X and Y together form an alkene, or C₃-C₅ cycloalkyl;
R₁ is —COOH;
R₂ is an electron withdrawing group;
R₅ is —(CH₂)ₖ—S(O)ᵢ—R₇, —NH—SO₂—R₇, —(CH₂)ₖ—W—R₈, —NH—(CZ₁)—R₈, —NH—(CZ₁)—NR₈, substituted aryl, substituted C₁₋₄alkyl, or substituted C₁₋₄alkenyl;
R₆ is selected from H, halo, HET, —CN, NH₂, NO₂, alkyl, substituted alkyl, alkoxy, substituted alkoxy, —NH—CO-HET, and —NH—CO-aryl;
R₇ is selected from alkyl, substituted alkyl, aryl, substituted aryl, —N(Q₁₅)₂, HET, and substituted HET;
R₈ is H, alkyl, substituted alkyl, aryl, substituted aryl, HET, substituted HET, cycloalkyl, substituted cycloalkyl;
Each Q₁₅ is independently alkyl, cycloalkyl, heterocycloalkyl, heteroaryl, phenyl, or naphthyl, each optionally substituted with 1-4 substituents independently selected from —F, —Cl, —Br, —I, —OQ₁₆, —SQ₁₆, —S(O)₂Q₁₆, —S(O)Q₁₆, —OS(O)₂Q₁₆, —C(═NQ₁₆)Q₁₆, —S(O)₂—N═S(O)(Q₁₆)₂, —S(O)₂—N═S(Q₁₆)₂, —SC(O)Q₁₆, —NQ₁₆Q₁₆, —C(O)Q₁₆, —C(S)Q₁₆, —C(O)OQ₁₆, —OC(O)Q₁₆, —C(O)NQ₁₆Q₁₆, —C(S)NQ₁₆Q₁₆, —C(O)C(Q₁₆)₂OC(O)Q₁₆, —CN, —NQ₁₆C(O)Q₁₆, —NQ₁₆C(S)Q₁₆, —NQ₁₆C(O)NQ₁₆Q₁₆, —NQ₁₆C(S)NQ₁₆Q₁₆, —S(O)₂NQ₁₆Q₁₆, —NQ₁₆S(O)₂Q₁₆, —NQ₁₆S(O)Q₁₆, —NQ₁₆SQ₁₆, —NO₂, and —SNQ₁₆Q₁₆. The alkyl, cycloalkyl, and cycloalkenyl being furher optionally substituted with ═O or ═S;
Each Q₁₆ is independently selected from —H, alkyl, and cycloalkyl. The alkyl and cycloalkyl optionally including 1-3 halos;
W is O, —(CZ₂)—, or —(CHZ₃)—;
Z₁ is O or S;
Z₂ is ═O, ═S , ═N—OH, ═N—O-alkyl, or ═N—O-substituted alkyl;
Z₃ is —OH, —N═NH, —N═N-alkyl, —NH-alkyl, or —NH-substituted alkyl;
i is 0, 1, or 2;
k is 0, 1, or 2 provided that when R₆ is H that R₅ is not attached to the phenyl ring at the ortho-position relative to Y, and further provided that R₅ is not 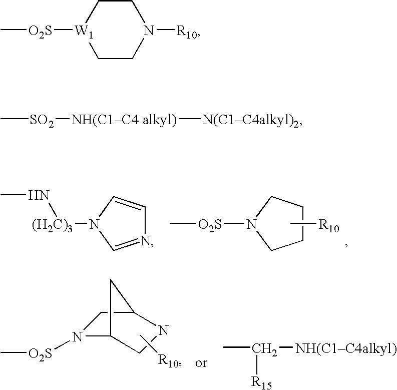
W₁ is N or CH;
R₁₀ is C₁-C₄ alkyl, C₁-C₄ substituted alkyl, Het, substituted Het, aryl, or substituted aryl; and
R₁₅ is H, C₁-C₄ alkyl, C₁-C₄ substituted alkyl, Het, substituted Het, C₄-C₇ cycloalkyl.

3. The compound of claim 1 having the formula 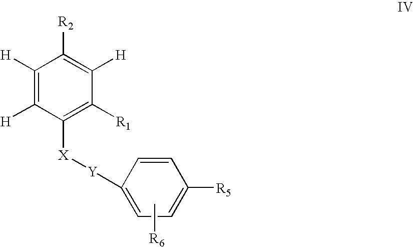
or a pharmaceutically acceptable salt thereof,
wherein
X═NH
Y═CO, CS, —C(═N—CN) or
X and Y together form an alkene, or C₃-C₅ cycloalkyl;
R₁ is —COOH;
R₂ is an electron withdrawing group;
R₅ is —CH₂)ₖ—S(O)ᵢ—R₇, —NH—SO₂—R₇, —(CH₂)ₖ—W—R₈, —NH—(CZ₁)—R₈, —NH—(CZ₁)—NR₈, substituted aryl, substituted C₁₋₄alkyl, or substituted C₁₋₄alkenyl;
R₆ is selected from H, halo, HET, —CN, NH₂, NO₂, alkyl, substituted alkyl, alkoxy, substituted alkoxy, —NH—CO-HET, and —NH—CO-aryl;
R₇ is selected from alkyl, substituted alkyl, aryl, substituted aryl, —N(Q₁₅)₂, HET, and substituted HET;
R₈ is H, alkyl, substituted alkyl, aryl, substituted aryl, HET, substituted HET, cycloalkyl, substituted cycloalkyl;
Each Q₁₅ is independently alkyl, cycloalkyl, heterocycloalkyl, heteroaryl, phenyl, or naphthyl, each optionally substituted with 1-4 substituents independently selected from —F, —Cl, —Br, —I, —OQ₁₆, —SQ₁₆, —S(O)₂Q₁₆, —S(O)Q₁₆, —OS(O)₂Q₁₆, —C(═NQ₁₆)Q₁₆, —S(O)₂—N═S(O)(Q₁₆)₂, —S(O)₂—N═S(Q₁₆)₂, —SC(O)Q₁₆, —NQ₁₆Q₁₆, —C(O)Q₁₆, —C(S)Q₁₆, —C(O)OQ₁₆, —OC(O)Q₁₆, —C(O)NQ₁₆Q₁₆, —C(S)NQ₁₆Q₁₆, —C(O)C(Q₁₆)₂OC(O)Q₁₆, —CN, —NQ₁₆C(O)Q₁₆, —NQ₁₆C(S)Q₁₆, —NQ₁₆C(O)NQ₁₆Q₁₆, —NQ₁₆C(S)NQ₁₆Q₁₆, —S(O)₂NQ₁₆Q₁₆, —NQ₁₆S(O)₂Q₁₆, —NQ₁₆S(O)Q₁₆, —NQ₁₆SQ₁₆, —NO₂, and —SNQ₁₆Q₁₆. The alkyl, cycloalkyl, and cycloalkenyl being furher optionally substituted with ═O or ═S;
Each Q₁₆ is independently selected from —H, alkyl, and cycloalkyl. The alkyl and cycloalkyl optionally including 1-3 halos;
W is O, —(CZ₂)—, or —(CHZ₃)—;
Z₁ is O or S;
Z₂ is ═O, ═S, ═N—OH, ═N—O-alkyl, or ═N—O-substituted alkyl;
Z₃ is —OH, —N═NH, —N═N-alkyl, —NH-alkyl, or —NH-substituted alkyl;
i is 0, 1, or 2;
k is 0, 1, or 2;
provided that R₅ is not 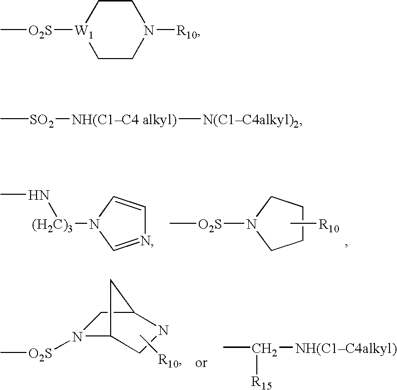
W₁ is N or CH;
R₁₀ is C₁-C₄ alkyl, C₁-C₄ substituted alkyl, Het, substituted Het, aryl, or substituted aryl; and
R₁₅ is H, C₁-C₄ alkyl, C₁-C₄ substituted alkyl, Het, substituted Het, C₄-C₇ cycloalkyl.

4. The compound of claim 1 having the formula 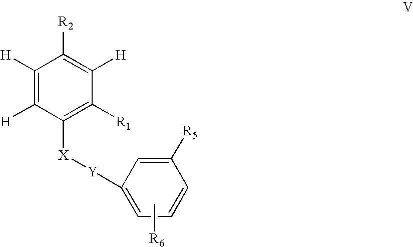
or a pharmaceutically acceptable salt thereof,
wherein
X═NH
Y═CO, CS, —C(═N—CN) or
X and Y together form an alkene, or C₃-C₅ cycloalkyl;
R₁ is —COOH;
R₂ is an electron withdrawing group;
R₅ is —(CH₂)ₖ—S(O)—ᵢ—R₇, —NH—SO₂—R₇, —(CH₂)ₖ—W—R₈, —NH—(CZ₁)—R₈, —NH—(CZ₁)—NR₈, substituted aryl, substituted C₁₋₄alkyl, or substituted C₁₋₄alkenyl;
R₆ is selected from H, halo, HET, —CN, NH₂, NO₂, alkyl, substituted alkyl, alkoxy, substituted alkoxy, —NH—CO-HET, and —NH—CO-aryl;
R₇ is selected from alkyl, substituted alkyl, aryl, substituted aryl, —N(Q₁₅)₂, HET, and substituted HET;
R₈ is H, alkyl, substituted alkyl, aryl, substituted aryl, HET, substituted HET, cycloalkyl, substituted cycloalkyl;
Each Q₁₅ is independently alkyl, cycloalkyl, heterocycloalkyl, heteroaryl, phenyl, or naphthyl, each optionally substituted with 1-4 substituents independently selected from —F, —Cl, —Br, —I, —OQ₁₆, —SQ₁₆, —S(O)₂Q₁₆, —S(O)Q₁₆, —OS(O)₂Q₁₆, —C(═NQ₁₆)Q₁₆, —S(O)₂—N═S(O)(Q₁₆)₂, —S(O)₂—N═S(Q₁₆)₂, —SC(O)Q₁₆, —NQ₁₆Q₁₆, —C(O)Q₁₆, —C(S)Q₁₆, —C(O)OQ₁₆, —OC(O)Q₁₆, —C(O)NQ₁₆Q₁₆, —C(S)NQ₁₆Q₁₆, —C(O)C(Q₁₆)₂OC(O)Q₁₆, —CN, —NQ₁₆C(O)Q₁₆, —NQ₁₆C(S)Q₁₆, —NQ₁₆C(O)NQ₁₆Q₁₆, —NQ₁₆C(S)NQ₁₆Q₁₆, —S(O)₂NQ₁₆Q₁₆, —NQ₁₆S(O)₂Q₁₆, —NQ₁₆S(O)Q₁₆, —NQ₁₆SQ₁₆, —NO₂, and —SNQ₁₆Q₁₆. The alkyl, cycloalkyl, and cycloalkenyl being furher optionally substituted with ═O or ═S;
Each Q₁₆ is independently selected from —H, alkyl, and cycloalkyl. The alkyl and cycloalkyl optionally including 1-3 halos;
W is O, S, —(CZ₂)—, or —(CHZ₃)—;
Z₁ is O;
Z₂ is ═O, ═S , ═N—OH, ═N—O-alkyl, or ═N—O-substituted alkyl;
Z₃ is —OH, —N═NH, —N═N-alkyl, —NH-alkyl, or —NH-substituted alkyl;
i is 0, 1, or 2;
k is 0, 1, or 2;
provided that R₅ is not 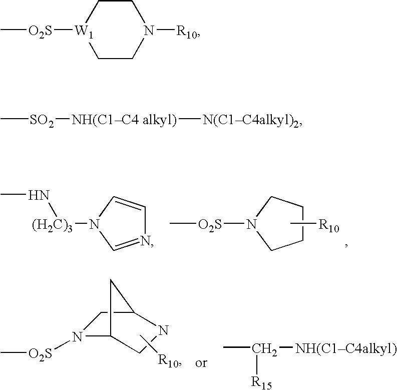
W₁ is Nor CH;
R₁₀ is C₁-C₄ alkyl, C₁-C₄ substituted alkyl, Het, substituted Het, aryl, or substituted aryl; and
R₁₅ is H, C₁-C₄ alkyl, C₁-C₄ substituted alkyl, Het, substituted Het, C₄-C₇ cycloalkyl.

5. The compound of claim 1, wherein Y is —CO—.

6. The compound of claim 1, wherein Y is —CS—.

7. The compound of claim 1, wherein X—Y is —C═C—.

8. The compound of claim 1, wherein X—Y is cyclopropyl.

9. The compound of claim 1, wherein R₂ is halo, —CN, —NO₂, HET, substituted HET, aryl, substituted aryl, —(CO)-alkyl, —(CO)-substituted alkyl, —(CO)-aryl, —(CO)-substituted aryl, —(CO)—O-alkyl, —(CO)—O-substituted alkyl, —(CO)—O-aryl, —(CO)—O-substituted aryl, —OC(Z,)₃, —C(Zn)₃,
—C(Zₙ)₂—O—C(Zₘ)₃, —SO₂—C(Zₙ)₃, —SO₂-aryl, —C(NQ₁₇)Q₁₇. —CH═C(Q₁₇)₂, —C≡C-Q₁₇, in which each Zn and Zm is independently H, halo, —CN, —NO₂—OH, or C₁₋₄alkyl optionally substituted with 1-3 halo,
—OH, NO₂, provided that at least one of Zn is halo, —CN, or NO₂, provided that R₂ is not pyridine or substituted pyridine.

10. The compound of claim 9, wherein R₂ is Br, Cl, F, I, CF₃, —CN, formyl, methoxyimino, hydroxyimino, —CH₂-halo, CH₂—CN, phenyl, thienyl, pyrazinyl, 1-methyl-1H-pyrrol-2-yl, chlorophenyl, nitrophenyl, cyanophenyl, chlorothienyl, methylthienyl, fluorophenyl, (trifluoromethy)phenyl, di(trifluoromethyl)phenyl, difluorophenyl, dimethylisoxazolyl, dimethoxypyrimidinyl.

11. The compound of claim 1, wherein R₅ is —SO₂—NH-alkyl, —SO₂—NH-substituted alkyl, —SO₂—NH-aryl, —SO₂—NH-substituted aryl, —SO₂—NH-HET, —SO₂—NH-substituted HET, —SO₂—N(alkyl)(substituted alkyl), —SO₂—N(alkyl)(aryl), —SO₂—N(alkyl)(substituted aryl); —SO₂—N(alkyl)(HET), —SO₂—N(alkyl)(substituted HET), —CH₂—SO₂-HET, —CH₂—SO₂-substituted HET, —S-alkyl, —S-substituted alkyl, —O-alkyl, —S-substituted alkyl, —CH₂—S-alkyl, —CH₂—S-substituted alkyl, —(CH₂)₂—S-alkyl, —(CH₂)₂—S-substituted alkyl, —C(O)-aryl, —C(O)H, —C(OH)-aryl, —C(N—OCH₃)-aryl, —C(N—OH)-aryl, —C(O)—C₁₋₆cycloalkyl, —NH—C(O)—O—C1-4alkyl, —NH—C(O)-aryl, —NH—C(O)-substituted aryl, —NH—C(O)-HET, —NH—C(O)-substituted HET, —NHC(O)NH-aryl, —NHC(O)NH-substituted aryl, —NHC(O)NH-het, —NHC(O)NH-substituted het; provided that R₅ is not 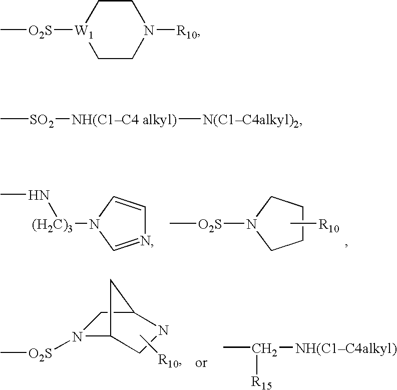
W₁ is N or CH;
R₁₀ is C₁-C₄ alkyl, C₁-C₄ substituted alkyl, Het, substituted Het, aryl, or substituted aryl; and
R₁₅ is H, C₁-C₄ alkyl, C₁-C₄ substituted alkyl, Het, substituted Het, C₄-C₇ cycloalkyl.

12. The compound of claim 11, wherein R₅ is (diethylamino)sulfonyl, (1H-indol-5-yl)aminosulfonyl, (furylmethylamino)sulfonyl, (ethoxycarbonyl)-1-piperazinylsulfonyl, pyridinylethylaminosulfonyl, (benzylamino)sulfonyl, (2-hydroxy-1-methylethyl)aminosulfonyl, (4-carboxyanilino)sulfonyl, (3,4-dihydro-1(2H)-quinolinyl)sulfonyl, [2-(3,5-dimethoxyphenyl)ethyl]aminosulfonyl, [(3S)-3-hydroxypyrrolidinyl]sulfonyl, (ethylanilino)sulfonyl, (3,5-dimethoxyanilino)sulfonyl, (2-hydroxy-2-phenylethyl)(methyl)amino]sulfonyl, (2,3-dihydro-1H-indol-1-yl)sulfonyl, (5-methoxy-2,3-dihydro-1H-indol-1-yl)sulfonyl, (5-fluoro-2,3-dihydro-1H-indol-1-yl)sulfonyl, (1H-benzimidazol-1-yl)sulfonyl, (5-fluoro-1H-indol-1-yl)sulfonyl, (1H-indol-1-yl)sulfonyl, (6-fluoro-1H-indol-1-yl)sulfonyl, (5-chloro-1H-indol-1-yl)sulfonyl, (6-chloro-1H-indol-1-yl)sulfonyl, (6-chloro-5-fluoro-1H-indol-1-yl)sulfonyl, (1H-pyrrol-1-yl)sulfonyl, (5-methoxy-1H-indol-1-yl)sulfonyl, (1H-pyrrolo[2,3-b]pyridin-1-yl)sulfonyl, (5-bromo-2,3-dihydro-1H-indol-1-yl)sulfonyl, (3,3-dimethyl-2,3-dihydro-1H-indol-1-yl)sulfonyl, (4-chlorophenyl)(methyl)amino]sulfonyl, benzylthio, methyl(pyridin-2-yl)amino]sulfonyl, (1H-indol-1-yl)sulfonyl, (pyrrolidin-1-yl)sulfonyl, (2-methylpyrrolidin-1-yl)sulfonyl, (morpholin-4-yl)sulfonyl, (piperidin-1-yl)sulfonyl, (methoxy-1H-indol-1-yl)sulfonyl, {methyl[(1R)-1-phenylethyl]amino}sulfonyl, {methyl[(1S)-1-phenylethyl]amino}sulfonyl, [(2-aminophenyl)(methyl)amino]sulfonyl, (dipropylamino)sulfonyl, benzylsulfanyl, (dipropylamino)sulfanyl, (dipropylamino)sulfinyl, [4-chloro(methyl)anilino]sulfonyl, (phenylthio)methyl, benzyloxy, 3-(ethylthio), (pyridin-4-ylmethyl)thio, phenoxy, phenylthio, (pyridin-4-ylmethyl)thio, benzylthio, (1-phenylethyl)thio, cyclopentylthio, cyclopentylsulfinyl, benzoyl, hydroxy(phenyl)methyl, (methoxyimino)(phenyl)methyl, (hydroxyimino)(phenyl)methyl, cyclopentylcarbonyl, benzoylamino, furoylamino, (thien-2-ylacetyl)amino, (mesitylcarbonyl)amino, (1,3-benzodioxol-5-ylcarbonyl)amino, 3-(2,4-dimethoxybenzoyl)amino, (phenylthio)acetylamino, (anilinocarbonyl)amino, (2,4-difluorophenyl)amino carbonylamino, (3-cyanophenyl)aminocarbonylamino, (3-acetylphenyl)aminocarbonylamino, -(trifluoromethoxy)phenylsulfonylamino, (thien-2-ylacetyl)amino, (5-nitro-2-furoyl)amino, (5-chloro-2-methoxyphenyl)aminocarbonylamino, (4-phenoxyphenyl)aminocarbonylamino, (4-acetylphenyl)aminocarbonylamino, phenylethynyl, 2-phenylethyl, and 1-pyrrolidinylsulfonyl)methyl.

13. The compound of claim 1, wherein P₆ is H, halo, —CN, NH₂, NO₂, methyl, methoxy, —(CH₂)₂—OH, morpholinyl, and —(CH₂)₂—O—CO—CH₃.

14. A compound of claim 1 having the structure
2-[(3-{[4-chloro(methyl)anilino]sulfonyl}benzoyl)amino]-5-(trifluoromethyl)benzoic acid;
5-bromo-2-({3-[(phenylsulfanyl)methyl]benzoyl}amino)benzoic acid;
2-{[3-(benzyloxy)benzoyl]amino}-5-bromobenzoic acid;
5-bromo-2-{[3-(ethylsulfanyl)benzoyl]amino}benzoic acid;
5-bromo-2-({3-[(4-pyridinylmethyl)sulfanyl]benzoyl}amino)benzoic acid;
5-bromo-2-({3-[(4-pyridinylmethyl)sulfanyl]benzoyl}amino)benzoic acid hydrochloride;
5-bromo-2-[(3-methoxybenzoyl)amino]benzoic acid;
2-[(3-bromo-5-{[(4-chlorophenyl)(methyl)amino]sulfonyl}benzoyl)amino]-5-chlorobenzoic acid;
5-bromo-2-[(4-methoxybenzoyl)amino]benzoic acid;
5-bromo-2-[(3-phenoxybenzoyl)amino]benzoic acid;
5-bromo-2-{[3-(methylsulfonyl)benzoyl]amino}benzoic acid;
2-[(3-benzoylbenzoyl)amino]-5-bromobenzoic acid;
5-bromo-2-[(3-{[4-chloro(methyl)anilino]sulfonyl}-5-nitrobenzoyl)amino]benzoic acid;
5-bromo-2-({3-bromo-5-[(5-bromo-2,3-dihydro-1H-indol-1-yl)sulfonyl]benzoyl}amino)benzoic acid;
5-bromo-2-[(4-{[methyl(2-pyridinyl)amino]sulfonyl}benzoyl)amino]benzoic acid;
2-[(4-{[2-amino(methyl)anilino]sulfonyl}benzoyl)amino]-5-bromobenzoic acid;
5-bromo-2-{[3-(1H-indol-1-ylsulfonyl)benzoyl]amino}benzoic acid;
2-[(3-benzoylbenzoyl)amino]-5-chlorobenzoic acid;
2-[(3-benzoylbenzoyl)amino]-5-nitrobenzoic acid;
2-[(4-acetylbenzoyl)amino]-5-bromobenzoic acid;
2-[(4-benzoylbenzoyl)amino]-5-bromobenzoic acid;
5-bromo-2-{[3-(2,3-dihydro-1H-indol-1-ylsulfonyl)benzoyl]amino}benzoic acid;
5-bromo-2-({3-[hydroxy(phenyl)methyl]benzoyl}amino)benzoic acid;
methyl 5-bromo-2-({3-[(E)-(methoxyimino)(phenyl)methyl]benzoyl}amino)benzoate;
2-[(3-acetylbenzoyl)amino]-5-bromobenzoic acid;
5-bromo-2-({3-[(E)-(methoxyimino)(phenyl)methyl]benzoyl}amino)benzoic acid;
2-({3-[(5-chloro-2,3-dihydro-1H-indol-1-yl)sulfonyl]benzoyl}amino)-5-cyanobenzoic acid;
3-[(3-benzoylbenzoyl)amino]-5-cyanobenzoic acid;
5-bromo-2-{[3-(phenylethynyl)benzoyl]amino}benzoic acid;
5-bromo-2-{[3-(phenylthio)benzoyl]amino}benzoic acid;
2-(benzoylamino)-5-cyanobenzoic acid;
5-bromo-2-{[3-cyano-5-(2,3-dihydro-1H-indol-1-ylsulfonyl)benzoyl]amino}benzoic acid;
5-bromo-2-{[3-(2-phenylethyl)benzoyl]amino}benzoic acid;
5-cyano-2-({3-[(hydroxyimino)(phenyl)methyl]benzoyl}amino)benzoic acid;
5-cyano-2-({3-[(methoxyimino)(phenyl)methyl]benzoyl}amino)benzoic acid;
5-cyano-2-[(3-phenoxybenzoyl)amino]benzoic acid;
5-cyano-2-({3-[(5-methoxy-1H-indol-1-yl)sulfonyl]benzoyl}amino)benzoic acid;
5-cyano-2-{[3-(pyrrolidin-1-ylsulfonyl)benzoyl]amino}benzoic acid;
5-cyano-2-{[3-(1H-indol-1-ylsulfonyl)benzoyl]amino}benzoic acid;
2-({3-[(5-chloro-1H-indol-1-yl)sulfonyl]benzoyl}amino)-5-cyanobenzoic acid;
5-cyano-2-{[3-(cyclopentylthio)benzoyl]amino}benzoic acid;
5-cyano-2-({3-[(1-phenylethyl)thio]benzoyl}amino)benzoic acid;
2-({3-[(5-chloro-2,3-dihydro-1H-indol-1-yl)sulfonyl]benzoyl}amino)-5-iodobenzoic acid;
5-cyano-2-({3-[(3,3-dimethyl-2,3-dihydro-1H-indol-1-yl)sulfonyl]benzoyl}amino)benzoic acid;
5-cyano-2-{[3-(2,3-dihydro-1H-indol-1-ylsulfonyl)-5-methylbenzoyl]amino}benzoic acid;
2-({3-[(5-chloro-2,3-dihydro-1H-indol-1-yl)sulfonyl]benzoyl}amino)-5-thien-2-ylbenzoic acid;
2-({3-[(5-chloro-2,3-dihydro-1H-indol-1-yl)sulfonyl]benzoyl}amino)-5-(1-methyl-1H-pyrrol-2-yl)benzoic acid;
2-({3-[(5-chloro-2,3-dihydro-1H-indol-1-yl)sulfonyl]benzoyl}amino)-5-pyrazin-2-ylbenzoic acid;
2-({3-[(5-chloro-2,3-dihydro-1H-indol-1-yl)sulfonyl]benzoyl}amino)-5-(2-furyl)benzoic acid;
4-({3-[(5-chloro-2,3-dihydro-1H-indol-1-yl)sulfonyl]benzoyl}amino)-1,1′-biphenyl-3-carboxylic acid;
5-cyano-2-[(4-phenoxybenzoyl)amino]benzoic acid;
5-cyano-2-{[3-(3-fluorophenoxy)benzoyl]amino}benzoic acid;
5-cyano-2-{[3-(2-methylphenoxy)benzoyl]amino}benzoic acid;
2-({[2-(4-chlorophenyl)-1,1-dioxido-3,4-dihydro-2H-1,2-benzothiazin-7-yl]carbonyl}amino)-5-cyanobenzoic acid;
5-cyano-2-{[3-({methyl[(1R)-1-phenylethyl]amino}sulfonyl)benzoyl]amino}benzoic acid;
5-cyano-2-{[3-( 55 methyl[(1S)-1-phenylethyl]amino}sulfonyl)benzoyl]amino}benzoic acid;
4′-chloro-4-({3-[(5-chloro-2,3-dihydro-1H-indol-1-yl)sulfonyl]benzoyl}amino)-1,1′-biphenyl-3-carboxylic acid;
4-({3-[(5-chloro-2,3-dihydro-1H-indol-1-yl)sulfonyl]benzoyl}amino)-3′-nitro-1,1′-biphenyl-3-carboxylic acid;
4-({3-[(5-chloro-2,3-dihydro-1H-indol-1-yl)sulfonyl]benzoyl}amino)-4′-cyano-1,1′-biphenyl-3-carboxylic acid;
2-({3-[(5-chloro-2,3-dihydro-1H-indol-1-yl)sulfonyl]benzoyl}amino)-5-(4-methylthien-2-yl)benzoic acid;
4-({3-[(5-chloro-2,3-dihydro-1H-indol-1-yl)sulfonyl]benzoyl}amino)-4′-fluoro-1,1′-biphenyl-3-carboxylic acid;
4-({3-[(5-chloro-2,3-dihydro-1H-indol-1-yl)sulfonyl]benzoyl}amino)-2′-(trifluoromethyl)-1,1′-biphenyl-3-carboxylic acid;
4-({3-[(5-chloro-2,3-dihydro-1H-indol-1-yl)sulfonyl]benzoyl}amino)-3′,5′-bis(trifluoromethyl)-1,1′-biphenyl-3-carboxylic acid;
4-({3-[(5-chloro-2,3-dihydro-1H-indol-1-yl)sulfonyl]benzoyl}amino)-2′,4′-difluoro-1,1′-biphenyl-3-carboxylic acid;
2-({3-[(5-chloro-2,3-dihydro-1H-indol-1-yl)sulfonyl]benzoyl}amino)-5-(5-methylthien-2-yl)benzoic acid;
4′-tert-butyl-4-({3-[(5-chloro-2,3-dihydro-1H-indol-1-yl)sulfonyl]benzoyl}amino)-1,1′-biphenyl-3-carboxylic acid;
4-({3-[(5-chloro-2,3-dihydro-1H-indol-1-yl)sulfonyl]benzoyl}amino)-3′-(trifluoromethyl)-1,1′-biphenyl-3-carboxylic acid;
4-({3-[(5-chloro-2,3-dihydro-1H-indol-1-yl)sulfonyl]benzoyl}amino)-4′-(trifluoromethyl)-1,1′-biphenyl-3-carboxylic acid;
2-({3-[(5-chloro-2,3-dihydro-1H-indol-1-yl)sulfonyl]benzoyl}amino)-5-pyridin-2-ylbenzoic acid;
5-cyano-2-{[3-(4-methoxyphenoxy)benzoyl]amino}benzoic acid;
5-cyano-2-{[3-(3-nitrophenoxy)benzoyl]amino}benzoic acid;
5-cyano-2-{[3-(piperidin-1-ylsulfonyl)benzoyl]amino}benzoic acid;
5-bromo-2-({3-[hydroxy(phenyl)methyl]benzoyl}amino)benzoic acid;
5-bromo-2-({3-[hydroxy(phenyl)methyl]benzoyl}amino)benzoic acid;
2-((E)-2-{3-[(5-chloro-2,3-dihydro-1H-indol-1-yl)sulfonyl]phenyl}ethenyl)-5-nitrobenzoic acid;
2-((Z)-2-{3-[(5-chloro-2,3-dihydro-1H-indol-1-yl)sulfonyl]phenyl}ethenyl)-4-nitrobenzoic acid;
4-({3-[(5-chloro-2,3-dihydro-1H-indol-1-yl)sulfonyl]benzoyl}amino)-2′-methyl-1,1′-biphenyl-3-carboxylic acid;
2-({3-[(5-chloro-2,3-dihydro-1H-indol-1-yl)sulfonyl]benzoyl}amino)-5-(5-chlorothien-2-yl)benzoic acid;
5-bromo-2-{[4-(pyrrolidin-1-ylsulfonyl)benzoyl]amino}benzoic acid;
5-bromo-2-{[3-(pyrrolidin-1-ylsulfonyl)benzoyl]amino}benzoic acid;
5-cyano-2-{[3-(morpholin-4-ylsulfonyl)benzoyl]amino}benzoic acid;
5-cyano-2-({3-[(7-methoxy-1H-indol-1-yl)sulfonyl]benzoyl}amino)benzoic acid;
5-cyano-2-({3-[(6-methoxy-1H-indol-1-yl)sulfonyl]benzoyl}amino)benzoic acid;
5-cyano-2-{[3-(2,3-dihydro-1H-indol-1-ylsulfonyl)-5-(2-hydroxyethyl)benzoyl]amino}benzoic acid;
5-cyano-2-({3-[(5-fluoro-1H-indol-1-yl)sulfonyl]benzoyl}amino)benzoic acid;
2-({3-[(5-chloro-2,3-dihydro-1H-indol-1-yl)sulfonyl]benzoyl}amino)-5-(2,4-dimethoxypyrimidin-5-yl)benzoic acid;
5-cyano-2-{[4-(phenylethynyl)benzoyl]amino}benzoic acid;
5-cyano-2-({3-[(pyridin-4-ylmethyl)thio]benzoyl}amino)benzoic acid;
5-cyano-2-({3-[(pyridin-4-ylmethyl)thio]benzoyl}amino)benzoic acid hydrochloride;
5-cyano-2-({3-[(2-methylpyrrolidin-1-yl)sulfonyl]benzoyl}amino)benzoic acid;
5-cyano-2-({3-[(2,5-dimethylpyrrolidin-1-yl)sulfonyl]benzoyl}amino)benzoic acid;
2-((Z)-2-{3-[(5-chloro-2,3-dihydro-1H-indol-1-yl)sulfonyl]phenyl}ethenyl)-5-cyanobenzoic acid;
2-((E)-2-{3-[(5-chloro-2,3-dihydro-1H-indol-1-yl)sulfonyl]phenyl}ethenyl)-5-cyanobenzoic acid;
2-{[3-(benzyloxy)benzoyl]amino}-5-cyanobenzoic acid;
2-({3-[(5-chloro-2,3-dihydro-1H-indol-1-yl)sulfonyl]benzoyl}amino)-5-(3,5-dimethylisoxazol-4-yl)benzoic acid;
2-[({3-[(5-chloro-2,3-dihydro-1H-indol-1-yl)sulfonyl]phenyl}carbonothioyl)amino]-5-cyanobenzoic acid;
5-cyano-2-{[3-(cyclopentylcarbonyl)benzoyl]amino}benzoic acid;
2-{[3-bromo-5-(2,3-dihydro-1H-indol-1-ylsulfonyl)benzoyl]amino}-5-chlorobenzoic acid;
5-chloro-2-{[3-(morpholin-4-ylsulfonyl)benzoyl]amino}benzoic acid;
5-bromo-2-{[3-(morpholin-4-ylsulfonyl)benzoyl]amino}benzoic acid;
2-{[3-(morpholin-4-ylsulfonyl)benzoyl]amino}-5-nitrobenzoic acid;
5-iodo-2-{[3-(morpholin-4-ylsulfonyl)benzoyl]amino}benzoic acid;
2-[(3-anilinobenzoyl)amino]-5-cyanobenzoic acid;
5-chloro-2-[(3-{[(4-chlorophenyl)(methyl)amino]sulfonyl}benzoyl)amino]benzoic acid;
2-{[3-(benzylthio)benzoyl]amino}-5-cyanobenzoic acid;
5-cyano-2-{[3-(cyclopentylsulfinyl)benzoyl]amino}benzoic acid;
2-{[(2-tert-butyl-1,3-dioxo-2,3-dihydro-1H-isoindol-5-yl)carbonyl]amino}-5-cyanobenzoic acid;
5-cyano-2-({3-[(pyrrolidin-1-ylsulfonyl)methyl]benzoyl}amino)benzoic acid;
2-{[3-bromo-5-(2,3-dihydro-1H-indol-1-ylsulfonyl)benzoyl]amino}-5-cyanobenzoic acid;
2-[(3-bromo-5-{[(4-chlorophenyl)(methyl)amino]sulfonyl}benzoyl)amino]-5-cyanobenzoic acid;
5-bromo-2-{[(2-tert-butyl-1,3-dioxo-2,3-dihydro-1H-isoindol-5-yl)carbonyl]amino}benzoic acid;
5-bromo-2-[(3-bromo-5-{[(4-chlorophenyl)(methyl)amino]sulfonyl}benzoyl)amino]benzoic acid;
2-{[3-bromo-5-(morpholin-4-ylsulfonyl)benzoyl]amino}-5-chlorobenzoic acid;
5-cyano-2-({[3-(morpholin-4-ylsulfonyl)phenyl]carbonothioyl}amino)benzoic acid;
5-cyano-2-({[3-(pyrrolidin-1-ylsulfonyl)phenyl]carbonothioyl}amino)benzoic acid;
5-(cyanomethyl)-2-{[3-(morpholin-4-ylsulfonyl)benzoyl]amino}benzoic acid;
5-chloro-2-{[3-(2,3-dihydro-1H-indol-1-ylsulfonyl)-5-methylbenzoyl]amino}benzoic acid;
5-formyl-2-{[3-(morpholin-4-ylsulfonyl)benzoyl]amino}benzoic acid;
2-[(3-{[(2-aminophenyl)(methyl)amino]sulfonyl}benzoyl)amino]-5-cyanobenzoic acid;
2-({3-[(5-chloro-2,3-dihydro-1H-indol-1-yl)sulfonyl]benzoyl}amino)-5-formylbenzoic acid;
2-({3-[(5-chloro-2,3-dihydro-1H-indol-1-yl)sulfonyl]benzoyl}amino)-5-[(E)-(methoxyimino)methyl]benzoic acid;
2-({3-[(5-chloro-2,3-dihydro-1H-indol-1-yl)sulfonyl]benzoyl}amino)-5-[(E)-(hydroxyimino)methyl]benzoic acid;
2-(2-{3-[(5-chloro-2,3-dihydro-1H-indol-1-yl)sulfonyl]phenyl}cyclopropyl)-5-cyanobenzoic acid;
5-chloro-2-((E)-2-{3-[(5-chloro-2,3-dihydro-1H-indol-1-yl)sulfonyl]phenyl}ethenyl)benzoic acid;
5-acetyl-2-{[3-(morpholin-4-ylsulfonyl)benzoyl]amino}benzoic acid;
2-{[3-(benzoylamino)benzoyl]amino}-5-bromobenzoic acid;
5-bromo-2-{[3-(2-furoylamino)benzoyl]amino}benzoic acid;
5-bromo-2-({3-[(thien-2-ylacetyl)amino]benzoyl}amino)benzoic acid;
5-bromo-2-({3-[(mesitylcarbonyl)amino]benzoyl}amino)benzoic acid;
5-bromo-2-({4-[(mesitylcarbonyl)amino]benzoyl}amino)benzoic acid;
2-({3-[(1,3-benzodioxol-5-ylcarbonyl)amino]benzoyl}amino)-5-bromobenzoic acid;
5-bromo-2-({3-[(2,4-dimethoxybenzoyl)amino]benzoyl}amino)benzoic acid;
5-bromo-2-[(3-{[(phenylthio)acetyl]amino}benzoyl)amino]benzoic acid;
5-bromo-2-({3-[(methoxyacetyl)amino]benzoyl}amino)benzoic acid;
2-({3-[(anilinocarbonyl)amino]benzoyl}amino)-5-bromobenzoic acid;
5-bromo-2-{[3-({[(2,4-difluorophenyl)amino]carbonyl}amino)benzoyl]amino}benzoic acid;
5-bromo-2-{[3-({[(3-cyanophenyl)amino]carbonyl}amino)benzoyl]amino}benzoic acid;
5-bromo-2-{[3-({[(3-chlorophenyl)amino]carbonyl}amino)benzoyl]amino}benzoic acid;
5-bromo-2-({3-[({[3-(methylthio)phenyl]amino}carbonyl)amino]benzoyl}amino)benzoic acid;
2-{(3-({[(3-acetylphenyl)amino]carbonyl}amino)benzoyl]amino}-5-bromobenzoic acid;
5-bromo-2-({4-[(phenylsulfonyl)amino]benzoyl}amino)benzoic acid;
5-bromo-2-{[3-({[4-(trifluoromethoxy)phenyl]sulfonyl}amino)benzoyl]amino}benzoic acid;
5-bromo-2-{[4-({[4-(trifluoromethoxy)phenyl]sulfonyl}amino)benzoyl]amino}benzoic acid;
5-bromo-2-[(4-{[(3,4-dichlorophenyl)sulfonyl]amino}benzoyl)amino]benzoic acid;
5-bromo-2-({4-[(thien-2-ylacetyl)amino]benzoyl}amino)benzoic acid;
5-bromo-2-({3-[(5-nitro-2-furoyl)amino]benzoyl}amino)benzoic acid;
5-bromo-2-({4-[(5-nitro-2-furoyl)amino]benzoyl}amino)benzoic acid;
5-bromo-2-{[4-({[(2,4-difluorophenyl)amino]carbonyl}amino)benzoyl]amino}benzoic acid;
5-bromo-2-{[3-({[(3,5-dichlorophenyl)amino]carbonyl}amino)benzoyl]amino}benzoic acid;
5-bromo-2-{[3-({[(5-chloro-2-methoxyphenyl)amino]carbonyl}amino)benzoyl]amino}benzoic acid;
5-bromo-2-{[3-({[(4-phenoxyphenyl)amino]carbonyl}amino)benzoyl]amino}benzoic acid;
5-bromo-2-{[4-({[(4-phenoxyphenyl)amino]carbonyl}amino)benzoyl]amino}benzoic acid;
2-{[3-({[(4-acetylphenyl)amino]carbonyl}amino)benzoyl]amino}-5-bromobenzoic acid;
8618 or 5-bromo-2-{[4-({[(4-nitrophenyl)amino]carbonothioyl}amino)benzoyl]amino}benzoic acid;
5-bromo-2-({3-[({[2-(trifluoromethyl)phenyl]amino}carbonothioyl)amino]benzoyl}amino)benzoic acid;
5-bromo-2-{[3-({[(3,4,5-trimethoxyphenyl)amino]carbonothioyl}amino)benzoyl]amino}benzoic acid;
5-bromo-2-({3-[({[3-(methylthio)phenyl]amino}carbonothioyl)amino]benzoyl}amino)benzoic acid;
2-{[3-({[(3-acetylphenyl)amino]carbonothioyl}amino)benzoyl]amino}-5-bromobenzoic acid;
5-bromo-2-({3-[(phenylsulfonyl)amino]benzoyl}amino)benzoic acid;
5-bromo-2-[(3-{[(3,4-dichlorophenyl)sulfonyl]amino}benzoyl)amino]benzoic acid;
5-bromo-2-[(4-{[(4-methylphenyl)sulfonyl]amino}benzoyl)amino]benzoic acid;
5-bromo-2-[(3-{[(4-chlorophenyl)(methyl)amino]sulfonyl}-2-methylbenzoyl)amino]benzoic acid;
5-acetyl-2-({3-[(5-chloro-2,3-dihydro-1H-indol-1-yl)sulfonyl]benzoyl}amino)benzoic acid;
2-({3-[(5-chloro-2,3-dihydro-1H-indol-1-yl)sulfonyl]-2-methylbenzoyl}amino)-5-cyanobenzoic acid;
5-bromo-2-[(3-{[(4-chlorophenyl)(methyl)amino]sulfonyl}-2-methoxybenzoyl)amino]benzoic acid;
5-bromo-2-({4-[(dimethylamino)sulfonyl]benzoyl}amino)benzoic acid;
5-bromo-2-({4-[(1H-indol-5-ylamino)sulfonyl]benzoyl}amino)benzoic acid;
5-bromo-2-({4-[(diethylamino)sulfonyl]benzoyl}amino)benzoic acid;
5-chloro-2-({4-[(dipropylamino)sulfonyl]benzoyl}amino)benzoic acid;
5-bromo-2-[(4-{[4-(ethoxycarbonyl)-1-piperazinyl]sulfonyl}benzoyl)amino]benzoic acid;
5-bromo-2-[(4-{[(2-furylmethyl)amino]sulfonyl}benzoyl)amino]benzoic acid;
5-bromo-2-{[4-({methyl[2-(2-pyridinyl)ethyl]amino}sulfonyl)benzoyl]amino}benzoic acid;
5-bromo-2-[(4-{[(3S)-3-hydroxypyrrolidinyl]sulfonyl}benzoyl)amino]benzoic acid;
2-({4-[(benzylamino)sulfonyl]benzoyl}amino)-5-bromobenzoic acid;
5-bromo-2-{[4-(2,3-dihydro-1H-indol-1-ylsulfonyl)benzoyl]amino}benzoic acid;
5-bromo-2-[(4-{[(2-hydroxy-1-methylethyl)amino]sulfonyl}benzoyl)amino]benzoic acid;
5-bromo-2-[(4-{[(4-carboxyphenyl)amino]sulfonyl}benzoyl)amino]benzoic acid;
5-bromo-2-({4-[3,4-dihydro-1(2H)-quinolinylsulfonyl]benzoyl}amino)benzoic acid;
5-bromo-2-{[4-({[2-(3,5-dimethoxyphenyl)ethyl]amino}sulfonyl)benzoyl]amino}benzoic acid;
5-bromo-2-({4-[(ethylanilino)sulfonyl]benzoyl}amino)benzoic acid;
5-bromo-2-[(4-{[(3,5-dimethoxyphenyl)amino]sulfonyl}benzoyl)amino]benzoic acid;
5-bromo-2-[(4-{[(2-hydroxy-2-phenylethyl)(methyl)amino]sulfonyl}benzoyl)amino]benzoic acid;
5-bromo-2-[(4-{[(4-chlorophenyl)(methyl)amino]sulfonyl}benzoyl)amino]benzoic acid;
5-chloro-2-({4-[(dipropylamino)thio]-3-nitrobenzoyl}amino)benzoic acid;
5-bromo-2-({4-[(5-fluoro-2,3-dihydro-1H-indol-1-yl)sulfonyl]benzoyl}amino)benzoic acid;
5-bromo-2-({4-[(5-methoxy-2,3-dihydro-1H-indol-1-yl)sulfonyl]benzoyl}amino)benzoic acid;
5-chloro-2-({4-[(dipropylamino)sulfonyl]-3-nitrobenzoyl}amino)benzoic acid;
5-bromo-2-[(3-{[4-chloro(methyl)anilino]sulfonyl}benzoyl)amino]benzoic acid;
5-bromo-2-{[4-(1H-indol-1-ylsulfonyl)benzoyl]amino}benzoic acid;
2-{[4-(1H-benzimidazol-1-ylsulfonyl)benzoyl]amino}-5-bromobenzoic acid;
5-chloro-2-{[4-(2,3-dihydro-1H-indol-1-ylsulfonyl)-3-nitrobenzoyl]amino}benzoic acid;
5-chloro-2-({3-(hydroxyamino)-4-[(1-propylbutyl)sulfonyl]benzoyl}amino)benzoic acid hydrochloride;
2-({3-amino-4-[(dipropylamino)sulfonyl]benzoyl}amino)-5-chlorobenzoic acid hydrochloride;
2-{[4-(benzylthio)-3-nitrobenzoyl]amino}-5-chlorobenzoic acid;
5-bromo-2-({3-[(5-chloro-2,3-dihydro-1H-indol-1-yl)sulfonyl]benzoyl}amino)benzoic acid;
5-bromo-2-[(5-{[(4-chlorophenyl)(methyl)amino]sulfonyl}-2-methoxybenzoyl)amino]benzoic acid;
5-bromo-2-[(5-{[(4-chlorophenyl)(methyl)amino]sulfonyl}-2-methylbenzoyl)amino]benzoic acid;
5-bromo-2-({4-[(2,3-dihydro-1H-indol-1-ylcarbonyl)amino]benzoyl}amino)benzoic acid;
2-{[4-(benzylsulfanyl)-3-bromobenzoyl]amino}-5-chlorobenzoic acid;
5-bromo-2-({4-[(5-chloro-1H-indol-1-yl)sulfonyl]benzoyl}amino)benzoic acid;
5-bromo-2-({4-[(6-chloro-1H-indol-1-yl)sulfonyl]benzoyl}amino)benzoic acid;
5-bromo-2-({4-[(6-chloro-5-fluoro-1H-indol-1-yl)sulfonyl]benzoyl}amino)benzoic acid;
5-bromo-2-({4-[(6-fluoro-1H-indol-1-yl)sulfonyl]benzoyl}amino)benzoic acid;
5-bromo-2-[(2-bromo-5-{[(4-chlorophenyl)(methyl)amino]sulfonyl}benzoyl)amino]benzoic acid;
5-bromo-2-({4-[(5-fluoro-1H-indol-1-yl)sulfonyl]benzoyl}amino)benzoic acid;
5-bromo-2-{[4-(1H-pyrrol-1-ylsulfonyl)benzoyl]amino}benzoic acid;
5-chloro-2-[(4-methoxy-3-nitrobenzoyl)amino]benzoic acid;
2-({3-bromo-4-[(dipropylamino)sulfonyl]benzoyl}amino)-5-chlorobenzoic acid;
5-bromo-2-({4-[(5-methoxy-1H-indol-1-yl)sulfonyl]benzoyl}amino)benzoic acid;
5-bromo-2-[(3-{[(4-chlorophenyl)(methyl)amino]sulfonyl}-4-methoxybenzoyl)amino]benzoic acid;
5-bromo-2-[(3-{[4-chloro(methyl)anilino]sulfonyl}-4-methylbenzoyl)amino]benzoic acid;
5-bromo-2-[(4-bromo-3-{[4-chloro(methyl)anilino]sulfonyl}benzoyl)amino]benzoic acid;
5-bromo-2-{[4-(1H-pyrrolo[2,3-b]pyridin-1-ylsulfonyl)benzoyl]amino}benzoic acid;
2-[(3-{[4-chloro(methyl)anilino]sulfonyl}benzoyl)amino]-5-nitrobenzoic acid;
2-[(4-{[4-chloro(methyl)anilino]sulfonyl}benzoyl)amino]-5-nitrobenzoic acid;
2-{[3-(benzylsulfanyl)benzoyl]amino}-5-bromobenzoic acid;
5-bromo-2-{[3-{[4-chloro(methyl)anilino]sulfonyl}-4-(4-morpholinyl)benzoyl]amino}benzoic acid;
5-bromo-2-{[4-cyano-3-(2,3-dihydro-1H-indol-1-ylsulfonyl)benzoyl]amino}benzoic acid;
2-[(4-{[4-chloro(methyl)anilino]sulfonyl}benzoyl)amino]-5-cyanobenzoic acid;
2-{[3-(1H-benzimidazol-1-ylsulfonyl)benzoyl]amino}-5-isocyanobenzoic acid;
5-isocyano-2-{[3-(1H-pyrrolo[2,3-b]pyridin-1-ylsulfonyl)benzoyl]amino}benzoic acid;
5-iodo-2-{[4-(1,3-thiazolidin-3-ylsulfonyl)benzoyl]amino}benzoic acid;
2-{[4-(2,5-dihydro-1H-pyrrol-1-ylsulfonyl)benzoyl]amino}-5-iodobenzoic acid;
2-({4-[(4-cyano-4-phenylpiperidin-1-yl)sulfonyl]benzoyl}amino)-5-iodobenzoic acid;
2-({3-[(6-chloro-2,3-dihydro-1H-indol-1-yl)sulfonyl]benzoyl}amino)-5-(5-methoxy-5-oxopentanoyl)benzoic acid;
2-({3-[(6-chloro-2,3-dihydro-1H-indol-1-yl)sulfonyl]benzoyl}amino)-5-(cyclopentylcarbonyl)benzoic acid;
5-(5-methoxy-5-oxopentanoyl)-2-{[3-(1,3-thiazolidin-3-ylsulfonyl)benzoyl]amino}benzoic acid;
5-iodo-2-{[3-(1,3-thiazolidin-3-ylsulfonyl)benzoyl]amino}benzoic acid;
2-{[3-(2,5-dihydro-1H-pyrrol-1-ylsulfonyl)benzoyl]amino}-5-iodobenzoic
5-iodo-2-({3-[(4-pyridin-2-ylpiperazin-1-yl)sulfonyl]benzoyl}amino)benzoic acid;
2-({4-[(6-chloro-2,3-dihydro-1H-indol-1-yl)sulfonyl]benzoyl}amino)-5-hexanoylbenzoic acid;
2-({4-[(6-chloro-2,3-dihydro-1H-indol-1-yl)sulfonyl]benzoyl}amino)-5-(cyclopentylcarbonyl)benzoic acid;
5-chloro-2-[(3-iodobenzoyl)amino]benzoic acid;
5-chloro-2-[(4-iodobenzoyl)amino]benzoic acid;
5-chloro-2-[(3-iodo-4-methylbenzoyl)amino]benzoic acid;
2-(benzoylamino)-5-chlorobenzoic acid;
2-[(4-benzoylbenzoyl)amino]-5-chlorobenzoic acid;
5-chloro-2-[(3-hexanoylbenzoyl)amino]benzoic acid;
5-chloro-2-[(4-hexanoylbenzoyl)amino]benzoic acid;
5-chloro-2-{[3-(3,4,5-trimethoxybenzoyl)benzoyl]amino}benzoic acid;
5-chloro-2-{[3-(4-chlorobenzoyl)benzoyl]amino}benzoic acid;
5-chloro-2-{[3-(4-methoxybenzoyl)benzoyl]amino}benzoic acid;
5-chloro-2-({3-[(4-chlorophenoxy)acetyl]benzoyl}amino)benzoic acid;
5-chloro-2-{[3-(cyclopentylcarbonyl)benzoyl]amino}benzoic acid;
5-chloro-2-{[3-(2-furoyl)benzoyl]amino}benzoic acid;
2-{[3-(1,3-benzodioxol-5-ylcarbonyl)benzoyl]amino}-5-chlorobenzoic acid;
5-chloro-2-({3-[4-(dimethylamino)benzoyl]benzoyl}amino)benzoic acid;
5-chloro-2-{[4-(3,4,5-trimethoxybenzoyl)benzoyl]amino}benzoic acid;
5-chloro-2-{[4-(4-chlorobenzoyl)benzoyl]amino}benzoic acid;
5-chloro-2-{[4-(4-methoxybenzoyl)benzoyl]amino}benzoic acid;
5-chloro-2-({4-[(4-chlorophenoxy)acetyl]benzoyl}amino)benzoic acid;
5-chloro-2-{[4-(cyclopentylcarbonyl)benzoyl]amino}benzoic acid;
5-chloro-2-{[4-(2-furoyl)benzoyl]amino}benzoic acid;
2-{[4-(1,3-benzodioxol-5-ylcarbonyl)benzoyl]amino}-5-chlorobenzoic acid;
5-chloro-2-({4-[4-(dimethylamino)benzoyl]benzoyl}amino)benzoic acid;
5-bromo-2-({3-[(E)-(hydroxyimino)(phenyl)methyl]benzoyl}amino)benzoic acid compound with N,N,N-triethylamine (1:1);
2-{[4-(aminomethyl)-3-(2,3-dihydro-1H-indol-1-ylsulfonyl)benzoyl]amino}-5-bromobenzoic acid hydrochloride;
2-{[3-(benzyloxy)benzoyl]amino}-5-bromobenzoic acid;
5-bromo-2-{[3-(pentyloxy)benzoyl]amino}benzoic acid;
2-{[3-(allyloxy)benzoyl]amino}-5-bromobenzoic acid;
5-bromo-2-[(3-isopropoxybenzoyl)amino]benzoic acid;
2-{[4-(benzyloxy)benzoyl]amino}-5-bromobenzoic acid;
5-bromo-2-{[4-(pentyloxy)benzoyl]amino}benzoic acid;
5-bromo-2-[(4-isopropoxybenzoyl)amino]benzoic acid;
2-{[3-({[2-(4-aminophenyl)ethyl]amino}sulfonyl)-4-methylbenzoyl]amino}-5-bromobenzoic acid;
2-{[4-({[2-(4-aminophenyl)ethyl]amino}sulfonyl)benzoyl]amino}-5-bromobenzoic acid;
5-bromo-2-({3-[(2,3-dihydro-1H-inden-2-ylamino)sulfonyl]-4-methylbenzoyl}amino)benzoic acid;
5-bromo-2-({3-[(2,3-dihydro-1H-inden-2-ylamino)sulfonyl]-4-methoxybenzoyl}amino)benzoic acid;
5-bromo-2-({2-bromo-5-[(2,3-dihydro-1H-inden-2-ylamino)sulfonyl]benzoyl}amino)benzoic acid;
5-bromo-2-({3-[(2,3-dihydro-1H-inden-2-ylamino)sulfonyl]benzoyl}amino)benzoic acid;
5-bromo-2-[(5-{[ethyl(pyridin-4-ylmethyl)amino]sulfonyl}-2-methylbenzoyl)amino]benzoic acid;
5-bromo-2-{[4-methyl-3-({4-[3-(trifluoromethyl)phenyl]piperazin-1-yl}sulfonyl)benzoyl]amino}benzoic acid;
5-bromo-2-{[4-bromo-3-({4-[3-(trifluoromethyl)phenyl]piperazin-1-yl}sulfonyl)benzoyl]amino}benzoic acid;
5-bromo-2-{[3-({4-[3-(trifluoromethyl)phenyl]piperazin-1-yl}sulfonyl)benzoyl]amino}benzoic acid;
5-bromo-2-[(5-{[4-(4-chlorophenyl)piperazin-1-yl]sulfonyl}-2-methylbenzoyl)amino]benzoic acid;
5-bromo-2-[(3-{[4-(4-chlorophenyl)piperazin-1-yl]sulfonyl}-4-methoxybenzoyl)amino]benzoic acid;
5-bromo-2-{[3-({[4-(dimethylamino)benzyl]amino}sulfonyl)-4-methoxybenzoyl]amino}benzoic acid;
5-bromo-2-({3-[(3,5-dimethoxybenzyl)oxy]benzoyl}amino)benzoic acid;
5-bromo-2-({3-[1-(ethoxycarbonyl)butoxy]benzoyl}amino)benzoic acid;
5-bromo-2-({4-[1-(ethoxycarbonyl)butoxy]benzoyl}amino)benzoic acid;
5-bromo-2-[(4-hydroxybenzoyl)amino]benzoic acid;
5-bromo-2-[(3-hydroxybenzoyl)amino]benzoic acid;
5-bromo-2-[(3-ethoxybenzoyl)amino]benzoic acid;
5-bromo-2-{[3-(2-methoxyethoxy)benzoyl]amino}benzoic acid;
5-bromo-2-({3-[2-(2-ethoxyethoxy)ethoxy]benzoyl}amino)benzoic acid;
5-bromo-2-{[3-(2-methoxy-2-oxo-1-phenylethoxy)benzoyl]amino}benzoic acid;
5-bromo-2-{[3-(2-methoxy-2-oxoethoxy)benzoyl]amino}benzoic acid;
5-bromo-2-({3-[2-(4-methyl-1,3-thiazol-5-yl)ethoxy]benzoyl}amino)benzoic acid;
5-bromo-2-{[3-(pyridin-3-ylmethoxy)benzoyl]amino}benzoic acid;
5-bromo-2-({3-[2-(2-oxopyrrolidin-1-yl)ethoxy]benzoyl}amino)benzoic acid;
5-bromo-2-{[3-(tetrahydrofuran-3-ylmethoxy)benzoyl]amino}benzoic acid;
5-bromo-2-({3-[2-(5-methyl-2-phenyl-1,3-oxazol-4-yl)ethoxy]benzoyl}amino)benzoic acid;
2-{[3-(1H-1,2,3-benzotriazol-1-ylmethoxy)benzoyl]amino}-5-bromobenzoic acid;
5-bromo-2-({3-[1-(ethoxycarbonyl)butoxy]benzoyl}amino)benzoic acid;
5-bromo-2-[(4-ethoxybenzoyl)amino]benzoic acid;
2-[(4-{2-[benzyl(methyl)amino]ethoxy}benzoyl)amino]-5-bromobenzoic acid;
5-bromo-2-{[4-(2-phenoxyethoxy)benzoyl]amino}benzoic acid;
5-bromo-2-{[4-(2-methoxyethoxy)benzoyl]amino}benzoic acid;
2-({4-[2-(acetylamino)ethoxy]benzoyl}amino)-5-bromobenzoic acid;
5-bromo-2-{[4-(hex-3-ynyloxy)benzoyl]amino}benzoic acid;
5-bromo-2-({4-[2-(2-ethoxyethoxy)ethoxy]benzoyl}amino)benzoic acid;
5-bromo-2-{[4-(2-methoxy-2-oxo-1-phenylethoxy)benzoyl]amino}benzoic acid;
5-bromo-2-({4-[(3-methoxybenzyl)oxy]benzoyl}amino)benzoic acid;
5-bromo-2-({4-[(3,5-dimethoxybenzyl)oxy]benzoyl}amino)benzoic acid;
5-bromo-2-{[4-(2-methoxy-2-oxoethoxy)benzoyl]amino}benzoic acid;
5-bromo-2-({4-[2-(4-methyl-1,3-thiazol-5-yl)ethoxy]benzoyl}amino)benzoic acid;
5-bromo-2-{[4-(pyridin-3-ylmethoxy)benzoyl]amino}benzoic acid;
5-bromo-2-({4-[2-(2-oxopyrrolidin-1-yl)ethoxy]benzoyl}amino)benzoic acid;
5-bromo-2-{[4-(2-methoxy-2-phenylethoxy)benzoyl]amino}benzoic acid;
5-bromo-2-{[4-(tetrahydrofuran-3-ylmethoxy)benzoyl]amino}benzoic acid;
2-{[4-(2-{[(benzyloxy)carbonyl]amino}ethoxy)benzoyl]amino}-5-bromobenzoic acid;
5-bromo-2-({4-[1-(ethoxycarbonyl)butoxy]benzoyl}amino)benzoic acid;
5-bromo-2-[(3-{[(4-methylphenyl)sulfonyl]amino}benzoyl)amino]benzoic acid;
5-chloro-2-({3-[(E)-2-furyl(hydroxyimino)methyl]benzoyl}amino)benzoic acid;
5-chloro-2-({3-[(E)-2-furyl(methoxyimino)methyl]benzoyl}amino)benzoic acid;
2-({4-[(E)-[(benzyloxy)imino](2-furyl)methyl]benzoyl}amino)-5-chlorobenzoic acid;
2-({4-[(E)-[(allyloxy)imino](2-furyl)methyl]benzoyl}amino)-5-chlorobenzoic acid;
5-chloro-2-({3-[[(3-chlorobenzyl)amino](2-furyl)methyl]benzoyl}amino)benzoic acid;
5-chloro-2-({3-[2-furyl(propylamino)methyl]benzoyl}amino)benzoic acid;
5-chloro-2-({3-[[(3-chlorobenzyl)amino](phenyl)methyl]benzoyl}amino)benzoic acid;
5-chloro-2-({3-[phenyl(propylamino)methyl]benzoyl}amino)benzoic acid;
5-chloro-2-({3-[(2,3-dihydro-1H-inden-1-ylamino)(phenyl)methyl]benzoyl}amino)benzoic acid;
5-chloro-2-({3-[(cyclopentylamino)(phenyl)methyl]benzoyl}amino)benzoic acid;
5-chloro-2-{[3-(2-ethylbutanoyl)benzoyl]amino}benzoic acid;
5-chloro-2-{[3-(tetrahydrofuran-2-ylcarbonyl)benzoyl]amino}benzoic acid;
5-chloro-2-{[3-(tetrahydrofuran-3-ylcarbonyl)benzoyl]amino}benzoic acid;
5-chloro-2-({3-[3-(methylsulfonyl)benzoyl]benzoyl}amino)benzoic acid;
5-chloro-2-({3-[(E)-(hydroxyimino)(pyridin-3-yl)methyl]benzoyl}amino)benzoic acid;
5-chloro-2-({3-[(E)-(hydroxyimino)(tetrahydrofuran-2-yl)methyl]benzoyl}amino)benzoic acid;
5-chloro-2-({3-[(3-cyanophenyl)(propylamino)methyl]benzoyl}amino)benzoic acid;
5-chloro-2-({3-[(propylamino)(pyridin-3-yl)methyl]benzoyl}amino)benzoic acid;
5-chloro-2-({3-[[3-(methylsulfonyl)phenyl](propylamino)methyl]benzoyl}amino)benzoic acid;
5-chloro-2-{[4-(cyclopentylcarbonyl)benzoyl]amino}benzoic acid;
5-chloro-2-{[4-(2-ethylbutanoyl)benzoyl]amino}benzoic acid;
5-chloro-2-{[4-(ethoxyacetyl)benzoyl]amino}benzoic acid;
5-chloro-2-{[4-(1H-indol-3-ylcarbonyl)benzoyl]amino}benzoic acid;
5-chloro-2-({4-[3-(methylsulfonyl)benzoyl]benzoyl}amino)benzoic acid;
5-chloro-2-({4-[(E)-(3-cyanophenyl)(hydroxyimino)methyl]benzoyl}amino)benzoic acid;
5-chloro-2-({4-[(1E)-2-ethoxy-N-hydroxyethanimidoyl]benzoyl}amino)benzoic acid;
5-chloro-2-({4-[(E)-(hydroxyimino)(pyridin-3-yl)methyl]benzoyl}amino)benzoic acid;
5-chloro-2-({4-[(E)-(hydroxyimino)(tetrahydrofuran-2-yl)methyl]benzoyl}amino)benzoic acid;
5-chloro-2-[(4-{(E)-(hydroxyimino)[3-(methylsulfonyl)phenyl]methyl}benzoyl)amino]benzoic acid;
5-chloro-2-({3-[(E)-2-furyl(hydroxyimino)methyl]benzoyl}amino)benzoic acid;
2-({4-[(E)-[(benzyloxy)imino](2-furyl)methyl]benzoyl}amino)-5-chlorobenzoic acid;
2-({4-[(E)-[(allyloxy)imino](2-furyl)methyl]benzoyl}amino)-5-chlorobenzoic acid;
2-[({2-[4-(acetyloxy)phenyl]-1,3-dioxo-2,3-dihydro-1H-isoindol-5-yl}carbonyl)amino]-5-bromobenzoic acid;
2-{[3-({[2-(4-aminophenyl)ethyl]amino}sulfonyl)-4-methoxybenzoyl]amino}-5-bromobenzoic acid;
2-{[3-({[2-(4-aminophenyl)ethyl]amino}sulfonyl)benzoyl]amino}-5-bromobenzoic acid;
5-bromo-2-{[3-({[3-(1H-imidazol-1-yl)propyl]amino}sulfonyl)benzoyl]amino}benzoic acid;
5-bromo-2-({5-[(2,3-dihydro-1H-inden-2-ylamino)sulfonyl]-2-methylbenzoyl}amino)benzoic acid;
5-bromo-2-({4-bromo-3-[(2,3-dihydro-1H-inden-2-ylamino)sulfonyl]benzoyl}amino)benzoic acid;
5-bromo-2-({4-[(2,3-dihydro-1H-inden-2-ylamino)sulfonyl]benzoyl}amino)benzoic acid;
5-bromo-2-[(3-{[ethyl(pyridin-4-ylmethyl)amino]sulfonyl}benzoyl)amino]benzoic acid;
5-bromo-2-[(4-{[ethyl(pyridin-4-ylmethyl)amino]sulfonyl}benzoyl)amino]benzoic acid;
5-bromo-2-{[2-bromo-5-({4-[3-(trifluoromethyl)phenyl]piperazin-1-yl}sulfonyl)benzoyl]amino}benzoic acid;
5-bromo-2-{[3-({[4-(dimethylamino)benzyl]amino}sulfonyl)-4-methylbenzoyl]amino}benzoic acid;
5-bromo-2-{[4-bromo-3-({[4-(dimethylamino)benzyl]amino}sulfonyl)benzoyl]amino}benzoic acid;
5-bromo-2-{[2-bromo-5-({[4-(dimethylamino)benzyl]amino}sulfonyl)benzoyl]amino}benzoic acid;
5-bromo-2-{[3-({[4-(dimethylamino)benzyl]amino}sulfonyl)benzoyl]amino}benzoic acid;
5-bromo-2-{[4-({[4-(dimethylamino)benzyl]amino}sulfonyl)benzoyl]amino}benzoic acid;
5-bromo-2-[(3-{[(4-chlorophenyl)(methyl)amino]sulfonyl}-4-methylbenzoyl)amino]benzoic acid;
5-bromo-2-[(3-{[(4-chloro-1-naphthyl)amino]sulfonyl}-4-methylbenzoyl)amino]benzoic acid;
5-bromo-2-{[3-(2,3-dihydro-1H-indol-1-ylsulfonyl)-4-methylbenzoyl]amino}benzoic acid;
5-bromo-2-{[3-(2,3-dihydro-1H-indol-1-ylsulfonyl)-4-methoxybenzoyl]amino}benzoic acid;
5-bromo-2-{[2-chloro-5-(2,3-dihydro-1H-indol-1-ylsulfonyl)-4-fluorobenzoyl]amino}benzoic acid;
5-bromo-2-({3-[(2,3-dihydro-1,4-benzodioxin-6-ylamino)sulfonyl]-4-methylbenzoyl amino)benzoic acid;
5-bromo-2-({5-[(2,3-dihydro-1,4-benzodioxin-6-ylamino)sulfonyl]-2-methylbenzoyl}amino)benzoic acid;
5-bromo-2-({3-[(2,3-dihydro-1,4-benzodioxin-6-ylamino)sulfonyl]-4-methoxybenzoyl}amino)benzoic acid;
5-bromo-2-({2-bromo-5-[(2,3-dihydro-1,4-benzodioxin-6-ylamino)sulfonyl]benzoyl}amino)benzoic acid;
5-bromo-2-({3-[(2,3-dihydro-1,4-benzodioxin-6-ylamino)sulfonyl]benzoyl}amino)benzoic acid;
5-bromo-2-({4-[(2,3-dihydro-1,4-benzodioxin-6-ylamino)sulfonyl]benzoyl}amino)benzoic acid;
5-bromo-2-[(3-{[(4-chlorophenyl)amino]sulfonyl}-4-methoxybenzoyl)amino]benzoic acid;
5-bromo-2-[(3-{[(4-morpholin-4-ylphenyl)amino]sulfonyl}benzoyl)amino]benzoic acid;
5-bromo-2-[(4-{[(4-morpholin-4-ylphenyl)amino]sulfonyl}benzoyl)amino]benzoic acid;
5-bromo-2-{[3-({[4-(diethylamino)phenyl]amino}sulfonyl)-4-methylbenzoyl]amino}benzoic acid;
5-bromo-2-{[4-bromo-3-({[4-(diethylamino)phenyl]amino}sulfonyl)benzoyl]amino}benzoic acid;
5-bromo-2-{[3-({[4-(diethylamino)phenyl]amino}sulfonyl)benzoyl]amino}benzoic acid;
5-bromo-2-{[4-({[4-(diethylamino)phenyl]amino}sulfonyl)benzoyl]amino}benzoic acid;
5-bromo-2-({3-[(6-chloro-2,3-dihydro-1H-indol-1-yl)sulfonyl]-4-methylbenzoyl}amino)benzoic acid;
5-bromo-2-({2-bromo-5-[(6-chloro-2,3-dihydro-1H-indol-1-yl)sulfonyl]benzoyl}amino)benzoic acid;
5-bromo-2-({3-[(6-chloro-2,3-dihydro-1H-indol-1-yl)sulfonyl]benzoyl}amino)benzoic acid;
5-bromo-2-({2-chloro-5-[(6-chloro-2,3-dihydro-1H-indol-1-yl)sulfonyl]-4-fluorobenzoyl}amino)benzoic acid;
5-bromo-2-({4-[(6-chloro-2,3-dihydro-1H-indol-1-yl)sulfonyl]benzoyl}amino)benzoic acid;
5-bromo-2-{[(9-oxo-9H-fluoren-4-yl)carbonyl]amino}benzoic acid;
5-bromo-2-[(2,5-difluorobenzoyl)amino]benzoic acid;
5-bromo-2-[(3-cyanobenzoyl)amino]benzoic acid;
5-bromo-2-[(3-bromobenzoyl)amino]benzoic acid;
5-bromo-2-[(3-fluorobenzoyl)amino]benzoic acid;
5-bromo-2-[(3-chlorobenzoyl)amino]benzoic acid;
5-bromo-2-[(3,5-dichlorobenzoyl)amino]benzoic acid;
5-bromo-2-{[3-(dimethylamino)benzoyl]amino}benzoic acid;
5-bromo-2-[(3,4-dimethoxybenzoyl)amino]benzoic acid;
5-bromo-2-[(3,4,5-trimethoxybenzoyl)amino]benzoic acid;
5-bromo-2-[(3,5-dimethoxybenzoyl)amino]benzoic acid;
5-bromo-2-({4-[(ethoxycarbonyl)oxy]-3,5-dimethoxybenzoyl}amino)benzoic acid;
5-bromo-2-[(3,4-diethoxybenzoyl)amino]benzoic acid;
5-bromo-2-[(3,4,5-triethoxybenzoyl)amino]benzoic acid;
5-bromo-2-{[3-(trifluoromethyl)benzoyl]amino}benzoic acid;
5-bromo-2-[(3-methylbenzoyl)amino]benzoic acid;
5-bromo-2-[(4-cyanobenzoyl)amino]benzoic acid;
5-bromo-2-[(4-fluorobenzoyl)amino]benzoic acid;
5-bromo-2-{[4-(dimethylamino)benzoyl]amino}benzoic acid;
5-bromo-2-{[4-(diethylamino)benzoyl]amino}benzoic acid;
5-bromo-2-[(4-butoxybenzoyl)amino]benzoic acid;
2-[(1,1′-biphenyl-4-ylcarbonyl)amino]-5-bromobenzoic acid;
5-bromo-2-{[4-(methylthio)benzoyl]amino}benzoic acid;
5-bromo-2-{[4-(ethylthio)benzoyl]amino}benzoic acid;
5-bromo-2-{[4-(methoxycarbonyl)benzoyl]amino}benzoic acid;
5-bromo-2-{[4-(trifluoromethyl)benzoyl]amino}benzoic acid;
5-bromo-2-[(3-methoxy-4-methylbenzoyl)amino]benzoic acid;
5-bromo-2-[(4-vinylbenzoyl)amino]benzoic acid;
5-bromo-2-(1-naphthoylamino)benzoic acid;
5-bromo-2-[(4-fluoro-1-naphthoyl)amino]benzoic acid;
5-bromo-2-(2-naphthoylamino)benzoic acid;
2-[(1,3-benzodioxol-5-ylcarbonyl)amino]-5-bromobenzoic acid;
5-bromo-2-[(4-formylbenzoyl)amino]benzoic acid;
5-bromo-2-[(3-nitrobenzoyl)amino]benzoic acid;
5-bromo-2-[(3-methoxy-4-nitrobenzoyl)amino]benzoic acid;
5-bromo-2-{[4-(methylsulfonyl)benzoyl]amino}benzoic acid;
5-bromo-2-{[2-chloro-5-(methylthio)benzoyl]amino}benzoic acid;
5-bromo-2-{[3-(methoxycarbonyl)-5-nitrobenzoyl]amino}benzoic acid;
5-bromo-2-{[(9-oxo-9H-fluoren-1-yl)carbonyl]amino}benzoic acid;
5-bromo-2-[(3,4-difluorobenzoyl)amino]benzoic acid;
5-bromo-2-[(4-propoxybenzoyl)amino]benzoic acid;
5-bromo-2-[(4,5-dimethoxy-2-nitrobenzoyl)amino]benzoic acid;
2-{[3-(acetyloxy)benzoyl]amino}-5-bromobenzoic acid;
5-bromo-2-[(2-bromo-4,5-dimethoxybenzoyl)amino]benzoic acid;
5-bromo-2-[(2-bromo-5-methoxybenzoyl)amino]benzoic acid;
5-bromo-2-[(3-formylbenzoyl)amino]benzoic acid;
5-bromo-2-[(5-fluoro-2-methylbenzoyl)amino]benzoic acid;
5-bromo-2-[(3-fluoro-4-methoxybenzoyl)amino]benzoic acid;
5-bromo-2-{[2-chloro-5-(trifluoromethyl)benzoyl]amino}benzoic acid;
5-bromo-2-{[(10,10-dioxido-9-oxo-9H-thioxanthen-3-yl)carbonyl]amino}benzoic acid;
5-bromo-2-({2-[(diethylamino)carbonyl]-3,6-difluorobenzoyl}amino)benzoic acid;
5-bromo-2-[(5-methoxy-2-nitrobenzoyl)amino]benzoic acid;
5-bromo-2-{[4-(difluoromethoxy)benzoyl]amino}benzoic acid;
5-cyano-2-[(3-hydroxybenzoyl)amino]benzoic acid;
5-cyano-2-[(4-hydroxybenzoyl)amino]benzoic acid;
5-bromo-2-({2-chloro-5-[(2,3-dihydro-1,4-benzodioxin-6-ylamino)sulfonyl]-4-fluorobenzoyl}amino)benzoic acid;
5-bromo-2-[(3-{[(4-chloro-1-naphthyl)amino]sulfonyl}-4-methoxybenzoyl)amino]benzoic acid;
5-bromo-2-[(2-bromo-5-{[(4-chlorophenyl)amino]sulfonyl}benzoyl)amino]benzoic acid;
5-bromo-2-[(4-methoxy-3-1[(4-morpholin-4-ylphenyl)amino]sulfonyl}benzoyl)amino]benzoic acid;
5-bromo-2-[(2-bromo-5-{[(3-chloro-4-fluorophenyl)amino]sulfonyl}benzoyl)amino]benzoic acid;
5-bromo-2-{[2-methyl-5-({4-[3-(trifluoromethyl)phenyl]piperazin-1-yl}sulfonyl)benzoyl]amino}benzoic acid;
5-bromo-2-{[2-bromo-5-(2,3-dihydro-1H-indol-1-ylsulfonyl)benzoyl]amino}benzoic acid;
5-bromo-2-[(2-chloro-5-{[(3-chloro-4-fluorophenyl)amino]sulfonyl}-4-fluorobenzoyl)amino]benzoic acid;
2-{[5-({[2-(4-aminophenyl)ethyl]amino}sulfonyl)-2-methylbenzoyl]amino}-5-bromobenzoic acid;
5-bromo-2-({4-bromo-3-[(2,3-dihydro-1,4-benzodioxin-6-ylamino)sulfonyl]benzoyl}amino)benzoic acid;
5-bromo-2-[(4-bromo-3-{[ethyl(pyridin-4-ylmethyl)amino]sulfonyl}benzoyl)amino]benzoic acid;
5-bromo-2-{[4-methoxy-3-({4-[3-(trifluoromethyl)phenyl]piperazin-1-yl}sulfonyl)benzoyl]amino}benzoic acid;
5-bromo-2-({4-[(E)-phenyldiazenyl]benzoyl}amino)benzoic acid;
5-bromo-2-{[(2-isobutyl-1,3-dioxo-2,3-dihydro-1H-isoindol-5-yl)carbonyl]amino}benzoic acid;
5-bromo-2-({[1,3-dioxo-2-(tetrahydrofuran-2-ylmethyl)-2,3-dihydro-1H-isoindol-5-yl]carbonyl}amino)benzoic acid;
5-bromo-2-[(4-methoxy-3-nitrobenzoyl)amino]benzoic acid;
5-bromo-2-[(2-fluorobenzoyl)amino]benzoic acid;
5-bromo-2-{[(2-methyl-1,3-dioxo-2,3-dihydro-1H-isoindol-5-yl)carbonyl]amino}benzoic acid;
5-cyano-2-[(3-methoxy-4-methylbenzoyl)amino]benzoic acid;
5-cyano-2-[(8-formyl-1-naphthoyl)amino]benzoic acid;
5-cyano-2-[(4-cyanobenzoyl)amino]benzoic acid;
5-cyano-2-{[3-(dimethylamino)benzoyl]amino}benzoic acid;
5-cyano-2-[(3-fluorobenzoyl)amino]benzoic acid;
5-cyano-2-[(4-vinylbenzoyl)amino]benzoic acid;
5-cyano-2-{[4-(ethylthio)benzoyl]amino}benzoic acid;
5-cyano-2-[(3,4,5-trimethoxybenzoyl)amino]benzoic acid;
5-cyano-2-{[4-(diethylamino)benzoyl]amino}benzoic acid;
2-[(1,1′-biphenyl-4-ylcarbonyl)amino]-5-cyanobenzoic acid;
5-cyano-2-[(3,4-dimethoxybenzoyl)amino]benzoic acid;
5-cyano-2-[(4,5-dimethoxy-2-nitrobenzoyl)amino]benzoic acid;
5-cyano-2-[(3,4-diethoxybenzoyl)amino]benzoic acid;
5-cyano-2-(2-naphthoylamino)benzoic acid;
5-cyano-2-[(4-ethoxybenzoyl)amino]benzoic acid;
5-cyano-2-[(3,4,5-triethoxybenzoyl)amino]benzoic acid;
5-cyano-2-{[(9-oxo-9H-fluoren-1-yl)carbonyl]amino}benzoic acid;
5-cyano-2-[(4-methoxybenzoyl)amino]benzoic acid;
5-cyano-2-{[4-(pentyloxy)benzoyl]amino}benzoic acid;
5-cyano-2-({2-[(diethylamino)carbonyl]-3,6-difluorobenzoyl}amino)benzoic acid;
5-cyano-2-[(3-methylbenzoyl)amino]benzoic acid;
5-cyano-2-[(4-propoxybenzoyl)amino]benzoic acid;
5-cyano-2-[(3-fluoro-4-methoxybenzoyl)amino]benzoic acid;
2-[(1,3-benzodioxol-5-ylcarbonyl)amino]-5-cyanobenzoic acid;
5-bromo-2-[(4-{[3-(trifluoromethyl)benzyl]oxy}benzoyl)amino]benzoic acid;
5-bromo-2-({4-[2-(4-methoxyphenyl)ethoxy]benzoyl}amino)benzoic acid;
5-bromo-2-[(3-{[3-(trifluoromethyl)benzyl]oxy}benzoyl)amino]benzoic acid;
5-bromo-2-({3-[2-(4-methoxyphenyl)ethoxy]benzoyl}amino)benzoic acid;
5-bromo-2-{[4-(cyclopentylmethoxy)benzoyl]amino}benzoic acid;
5-bromo-2-({3-[(3-nitrobenzyl)oxy]benzoyl}amino)benzoic acid;
5-bromo-2-{[3-(cyclopentylmethoxy)benzoyl]amino}benzoic acid;
5-bromo-2-({3-[(3-fluorobenzyl)oxy]benzoyl}amino)benzoic acid;
5-bromo-2-({4-[2-(2,5-dioxopyrrolidin-1-yl)ethoxy]benzoyl}amino)benzoic acid;
5-bromo-2-({4-[(3-fluorobenzyl)oxy]benzoyl}amino)benzoic acid;
5-bromo-2-{[3-(pyridin-4-ylmethoxy)benzoyl]amino}benzoic acid;
5-bromo-2-({4-[(3-nitrobenzyl)oxy]benzoyl}amino)benzoic acid;
5-bromo-2-{[4-(pyridin-4-ylmethoxy)benzoyl]amino}benzoic acid;
5-bromo-2-({4-[(3-methoxy-4-nitrobenzyl)oxy]benzoyl}amino)benzoic acid;
5-bromo-2-({4-[(3,5-dimethyl-1H-pyrazol-1-yl)methoxy]benzoyl}amino)benzoic acid;
5-bromo-2-({4-[(1-ethylprop-2-ynyl)oxy]benzoyl}amino)benzoic acid;
5-bromo-2-[(4-{[4-(methoxycarbonyl)benzyl]oxy}benzoyl)amino]benzoic acid;
5-bromo-2-[(4-{2-[ethyl(phenyl)amino]ethoxy}benzoyl)amino]benzoic acid;
5-bromo-2-{[4-(2-pyridin-2-ylethoxy)benzoyl]amino}benzoic acid;
5-bromo-2-({4-[2-(2-methyl-5-nitro-1H-imidazol-1-yl)ethoxy]benzoyl}amino)benzoic acid;
5-bromo-2-{[4-(tetrahydro-2H-pyran-4-yloxy)benzoyl]amino}benzoic acid;
5-bromo-2-({4-[(1-methylpyrrolidin-2-yl)methoxy]benzoyl}amino)benzoic acid;
5-bromo-2-({4-[2-(1,3-dioxo-1,3-dihydro-2H-isoindol-2-yl)ethoxy]benzoyl}amino)benzoic acid;
5-bromo-2-{[4-(but-2-ynyloxy)benzoyl]amino}benzoic acid;
5-bromo-2-{[4-(3,3-dimethylbutoxy)benzoyl]amino}benzoic acid;
5-bromo-2-{[4-(2-isopropoxyethoxy)benzoyl]amino}benzoic acid;
2-{[4-(2-anilinoethoxy)benzoyl]amino}-5-bromobenzoic acid;
2-({4-[2-(1,3-benzothiazol-2-ylthio)ethoxy]benzoyl}amino)-5-bromobenzoic acid;
5-bromo-2-[(4-{[4-(ethoxycarbonyl)cyclohexyl]oxy}benzoyl)amino]benzoic acid;
5-bromo-2-({4-[2-(3-nitrophenyl)ethoxy]benzoyl}amino)benzoic acid;
2-{[(4′-tert-butyl-1,1′-biphenyl-4-yl)carbonyl]amino}-5-cyanobenzoic acid;
5-cyano-2-{[4-(methylthio)benzoyl]amino}benzoic acid;
5-cyano-2-[(3-methoxybenzoyl)amino]benzoic acid;
5-cyano-2-[(3,5-dimethoxybenzoyl)amino]benzoic acid;
2-[(2-bromo-5-methoxybenzoyl)amino]-5-cyanobenzoic acid;
5-cyano-2-[(4-fluorobenzoyl)amino]benzoic acid;
2-[(3-chlorobenzoyl)amino]-5-cyanobenzoic acid;
5-cyano-2-(1-naphthoylamino)benzoic acid;
5-cyano-2-{[4-(dimethylamino)benzoyl]amino}benzoic acid;
5-cyano-2-[(3-{[2-(methoxymethyl)pyrrolidin-1-yl]sulfonyl}benzoyl)amino]benzoic acid;
5-cyano-2-{[3-(2,3-dihydro-1H-indol-1-ylsulfonyl)benzoyl]amino}benzoic acid;
5-cyano-2-({3-[(2,6-dimethylmorpholin-4-yl)sulfonyl]benzoyl}amino)benzoic acid;
5-cyano-2-({3-[(diisobutylamino)sulfonyl]benzoyl}amino)benzoic acid;
2-{[3-(azetidin-1-ylsulfonyl)benzoyl]amino}-5-cyanobenzoic acid;
5-cyano-2-[(4-{[methyl(2-phenylethyl)amino]sulfonyl}benzoyl)amino]benzoic acid;
5-cyano-2-({3-[(4-pyrimidin-2-ylpiperazin-1-yl)sulfonyl]benzoyl}amino)benzoic acid;
2-[(3-{[4-(4-acetylphenyl)piperazin-1-yl]sulfonyl}benzoyl)amino]-5-cyanobenzoic acid;
2-{[4-(azepan-1-ylsulfonyl)benzoyl]amino}-5-cyanobenzoic acid;
5-cyano-2-{[3-({3-[(diethylamino)carbonyl]piperidin-1-yl}sulfonyl)benzoyl]amino}benzoic acid;
5-cyano-2-({4-[(2,6-dimethylmorpholin-4-yl)sulfonyl]benzoyl}amino)benzoic acid;
5-cyano-2-[(4-{[(2-hydroxy-1-methyl-2-phenylethyl)(methyl)amino]sulfonyl}benzoyl)amino]benzoic acid;
5-cyano-2-[(3-{[cyclohexyl(methyl)amino]sulfonyl}benzoyl)amino]benzoic acid;
5-cyano-2-({4-[(4-cyano-4-phenylpiperidin-1-yl)sulfonyl]benzoyl}amino)benzoic acid;
5-cyano-2-({4-[(2-ethylpiperidin-1-yl)sulfonyl]benzoyl}amino)benzoic acid;
5-cyano-2-({3-[(6,7-dimethoxy-3,4-dihydroisoquinolin-2(1H)-yl)sulfonyl]benzoyl}amino)benzoic acid;
5-cyano-2-[(3-{[(3-hydroxy-3-phenylpropyl)(methyl)amino]sulfonyl}benzoyl)amino]benzoic acid;
2-[(3-{[butyl(ethyl)amino]sulfonyl}benzoyl)amino]-5-cyanobenzoic acid;
5-cyano-2-({4-[(6,7-dimethoxy-3,4-dihydroisoquinolin-2(1H)-yl)sulfonyl]benzoyl}amino)benzoic acid;
5-cyano-2-[(4-{[4-(4-nitrophenyl)piperazin-1-yl]sulfonyl}benzoyl)amino]benzoic acid;
5-cyano-2-{[4-({3-[(diethylamino)carbonyl]piperidin-1-yl}sulfonyl)benzoyl]amino}benzoic acid;
2-[(4-{[butyl(cyanomethyl)amino]sulfonyl}benzoyl)amino]-5-cyanobenzoic acid;
2-[(4-{[benzyl(isopropyl)amino]sulfonyl}benzoyl)amino]-5-cyanobenzoic acid;
5-cyano-2-({4-[(2,3-dihydro-1H-inden-5-ylamino)sulfonyl]benzoyl}amino)benzoic acid;
5-cyano-2-({3-[(4-phenylpiperazin-1-yl)sulfonyl]benzoyl}amino)benzoic acid;
2-[(4-{[butyl(ethyl)amino]sulfonyl}benzoyl)amino]-5-cyanobenzoic acid;
5-cyano-2-[(3-{[methyl(2-phenylethyl)amino]sulfonyl}benzoyl)amino]benzoic acid;
2-[(3-{[benzyl(isopropyl)amino]sulfonyl}benzoyl)amino]-5-cyanobenzoic acid;
5-cyano-2-({3-[(4-hydroxypiperidin-1-yl)sulfonyl]benzoyl}amino)benzoic acid;
2-{[3-(azepan-1-ylsulfonyl)benzoyl]amino}-5-cyanobenzoic acid;
5-cyano-2-[(3-{[(2-hydroxy-1-methyl-2-phenylethyl)(methyl)amino]sulfonyl}benzoyl)amino]benzoic acid;
5-cyano-2-[(4-{[(2-hydroxy-1-methyl-2-phenylethyl)(methyl)amino]sulfonyl}benzoyl)amino]benzoic acid;
5-cyano-2-[(3-{[4-(4-fluorophenyl)piperazin-1-yl]sulfonyl}benzoyl)amino]benzoic acid;
5-cyano-2-({4-[(4-hydroxypiperidin-1-yl)sulfonyl]benzoyl}amino)benzoic acid;
5-cyano-2-[(4-{[(2-cyanoethyl)(methyl)amino]sulfonyl}benzoyl)amino]benzoic acid;
5-cyano-2-({4-[(3,5-dimethylpiperidin-1-yl)sulfonyl]benzoyl}amino)benzoic acid;
5-cyano-2-{[3-(octahydroquinolin-1(2H)-ylsulfonyl)benzoyl]amino}benzoic acid;
2-[(3-{[butyl(cyanomethyl)amino]sulfonyl}benzoyl)amino]-5-cyanobenzoic acid;
2-[(3-{[benzyl(2-cyanoethyl)amino]sulfonyl}benzoyl)amino]-5-cyanobenzoic acid;
2-{[4-(azetidin-1-ylsulfonyl)benzoyl]amino}-5-cyanobenzoic acid;
5-cyano-2-({4-[(4-methylpiperidin-1-yl)sulfonyl]benzoyl}amino)benzoic acid;
5-cyano-2-({4-[(3-methylpiperidin-1-yl)sulfonyl]benzoyl}amino)benzoic acid;
5-cyano-2-[(4-{[(2-hydroxy-1-methyl-2-phenylethyl)(methyl)amino]sulfonyl}benzoyl)amino]benzoic acid;
5-cyano-2-({3-[(3,5-dimethylpiperidin-1-yl)sulfonyl]benzoyl}amino)benzoic acid;
2-[(4-{[benzyl(2-cyanoethyl)amino]sulfonyl}benzoyl)amino]-5-cyanobenzoic acid;
5-cyano-2-{[4-(octahydroquinolin-1(2H)-ylsulfonyl)benzoyl]amino}benzoic acid;
2-[(4-{[allyl(cyclopentyl)amino]sulfonyl}benzoyl)amino]-5-cyanobenzoic acid;
5-cyano-2-{[4-(morpholin-4-ylsulfonyl)benzoyl]amino}benzoic acid;
5-cyano-2-[(4-{[2-(methoxymethyl)pyrrolidin-1-yl]sulfonyl}benzoyl)amino]benzoic acid;
5-cyano-2-[(3-{[(2-hydroxy-2-phenylethyl)(methyl)amino]sulfonyl}benzoyl)amino]benzoic acid;
5-cyano-2-[(3-{[(2-hydroxy-1-methyl-2-phenylethyl)(methyl)amino]sulfonyl}benzoyl)amino]benzoic acid;
5-cyano-2-({3-[(4-cyano-4-phenylpiperidin-1-yl)sulfonyl]benzoyl}amino)benzoic acid;
5-cyano-2-({3-[(2-ethylpiperidin-1-yl)sulfonyl]benzoyl}amino)benzoic acid;
5-cyano-2-[(4-{[cyclohexyl(methyl)amino]sulfonyl}benzoyl)amino]benzoic acid;
5-cyano-2-{[4-(3,4-dihydroisoquinolin-2(1H)-ylsulfonyl)benzoyl]amino}benzoic acid;
5-cyano-2-{[3-({4-[3-(trifluoromethyl)phenyl]piperazin-1-yl}sulfonyl)benzoyl]amino}benzoic acid;
5-cyano-2-{[3-(3,4-dihydroisoquinolin-2( 1H)-ylsulfonyl)benzoyl]amino}benzoic acid;
5-cyano-2-({3-[(3-methylpiperidin-1-yl)sulfonyl]benzoyl}amino)benzoic acid;
5-cyano-2-[(3-{[(2-cyanoethyl)(methyl)amino]sulfonyl}benzoyl)amino]benzoic acid;
5-cyano-2-[(3-{[(2-hydroxy-1-methyl-2-phenylethyl)(methyl)amino]sulfonyl}benzoyl)amino]benzoic acid;
5-cyano-2-({3-[(4-methylpiperidin-1-yl)sulfonyl]benzoyl}amino)benzoic acid;
5-bromo-2-({3-[(5-oxopyrrolidin-2-yl)methoxy]benzoyl}amino)benzoic acid;
5-bromo-2-({3-[(5-oxopyrrolidin-2-yl)methoxy]benzoyl}amino)benzoic acid;
5-bromo-2-({3-[(3-methoxy-4-nitrobenzyl)oxy]benzoyl}amino)benzoic acid;
5-bromo-2-[(3-{[4-(methoxycarbonyl)benzyl]oxy}benzoyl)amino]benzoic acid;
5-bromo-2-({3-[(2-oxotetrahydrofuran-3-yl)oxy]benzoyl}amino)benzoic acid;
5-bromo-2-[(3-{2-[(tert-butoxycarbonyl)amino]-2-phenylethoxy}benzoyl)amino]benzoic acid;
5-bromo-2-[(3-{[4-(ethoxycarbonyl)cyclohexyl]oxy}benzoyl)amino]benzoic acid;
2-({4-[(1-allylbut-3-enyl)oxy]benzoyl}amino)-5-bromobenzoic acid;
4-{[3-(morpholin-4-ylsulfonyl)benzoyl]amino}-4′-(trifluoromethyl)-1,1′-biphenyl-3-carboxylic acid;
4′-methoxy-4-{[3-(morpholin-4-ylsulfonyl)benzoyl]amino}-1,1′-biphenyl-3-carboxylic acid;
4-{[3-(morpholin-4-ylsulfonyl)benzoyl]amino}-1,1′-biphenyl-3-carboxylic acid;
5-cyano-2-{[3-(cyclopentylsulfonyl)benzoyl]amino}benzoic acid;
5-cyano-2-({3-[(2,3-dihydro-1H-inden-1-ylamino)sulfonyl]benzoyl}amino)benzoic acid;
2-[(3-{[benzyl(2-hydroxyethyl)amino]sulfonyl}benzoyl)amino]-5-cyanobenzoic acid;
5-cyano-2-[(4-{[(2-hydroxy-2-phenylethyl)(methyl)amino]sulfonyl}benzoyl)amino]benzoic acid;
5-cyano-2-[(3-{[4-(4-nitrophenyl)piperazin-1-yl]sulfonyl}benzoyl)amino]benzoic acid;
5-cyano-2-({4-[(diisobutylamino)sulfonyl]benzoyl}amino)benzoic acid;
4′-chloro-4-{[3-(morpholin-4-ylsulfonyl)benzoyl]amino}-1,1′-biphenyl-3-carboxylic acid;
2-[(3-{[butyl(2-hydroxyethyl)amino]sulfonyl}benzoyl)amino]-5-cyanobenzoic acid;
5-cyano-2-({3-[(2,3-dihydro-1H-inden-5-ylamino)sulfonyl]benzoyl}amino)benzoic acid;
5-cyano-2-[(4-{[(3-hydroxy-3-phenylpropyl)(methyl)amino]sulfonyl}benzoyl)amino]benzoic acid;
2-[(4-{[benzyl(2-hydroxyethyl)amino]sulfonyl}benzoyl)amino]-5-cyanobenzoic acid;
5-cyano-2-[(3-{[2-(hydroxymethyl)pyrrolidin-1-yl]sulfonyl}benzoyl)amino]benzoic acid;
5-cyano-2-({4-[(2,3-dihydro-1H-inden-1-ylamino)sulfonyl]benzoyl}amino)benzoic acid;
2-[(4-{[butyl(2-hydroxyethyl)amino]sulfonyl}benzoyl)amino]-5-cyanobenzoic acid;
5-cyano-2-[(4-{[2-(hydroxymethyl)pyrrolidin-1-yl]sulfonyl}benzoyl)amino]benzoic acid;
2-[(3-{[allyl(cyclopentyl)amino]sulfonyl}benzoyl)amino]-5-cyanobenzoic acid;
5-cyano-2-({4-[(2-methylpyrrolidin-1-yl)sulfonyl]benzoyl}amino)benzoic acid;
5-cyano-2-[(3-{[methyl(2-pyridin-2-ylethyl)amino]sulfonyl}benzoyl)amino]benzoic acid;
5-chloro-2-{[3-(quinolin-2-ylcarbonyl)benzoyl]amino}benzoic acid;
5-chloro-2-{[3-(phenoxyacetyl)benzoyl]amino}benzoic acid;
5-chloro-2-({4-[3-(methoxycarbonyl)benzoyl]benzoyl}amino)benzoic acid;
5-chloro-2-[(3-isobutyrylbenzoyl)amino]benzoic acid;
5-chloro-2-{[3-(2-methoxybenzoyl)benzoyl]amino}benzoic acid;
5-chloro-2-({3-[3-(methoxycarbonyl)benzoyl]benzoyl}amino)benzoic acid;
5-chloro-2-{[3-(thien-2-ylcarbonyl)benzoyl]amino}benzoic acid;
5-chloro-2-{[3-(cyclobutylcarbonyl)benzoyl]amino}benzoic acid;
5-chloro-2-{[3-(3,3-dimethylbutanoyl)benzoyl]amino}benzoic acid;
5-chloro-2-{[3-(cyclopropylcarbonyl)benzoyl]amino}benzoic acid;
5-chloro-2-{[3-(3-cyclopentylpropanoyl)benzoyl]amino}benzoic acid;
5-chloro-2-{[3-(3,4-difluorobenzoyl)benzoyl]amino}benzoic acid;
5-chloro-2-{[3-(3,5-difluorobenzoyl)benzoyl]amino}benzoic acid;
5-chloro-2-{[3-(3-methylbenzoyl)benzoyl]amino}benzoic acid;
5-cyano-2-({4-[(pyrrolidin-1-ylsulfonyl)methyl]benzoyl}amino)benzoic acid;
5-cyano-2-({4-[(morpholin-4-ylsulfonyl)methyl]benzoyl}amino)benzoic acid;
5-cyano-2-{[3-(morpholin-4-ylcarbonyl)benzoyl]amino}benzoic acid;
5-[(E)-(hydroxyimino)methyl]-2-{[3-(morpholin-4-ylsulfonyl)benzoyl]amino}benzoic acid;
5-[(E)-(methoxyimino)methyl]-2-{[3-(morpholin-4-ylsulfonyl)benzoyl]amino}benzoic acid;
2-{[3-(2,3-dihydro-1H-indol-1-ylsulfonyl)benzoyl]amino}-5-(3-hydroxyprop-1-ynyl)benzoic acid;
5-acetyl-2-({4-[(5-chloro-2,3-dihydro-1H-indol-1-yl)sulfonyl]benzoyl}amino)benzoic acid;
2-{[4-(benzoylamino)benzoyl]amino}-5-bromobenzoic acid;
5-bromo-2-{[4-(2-furoylamino)benzoyl]amino}benzoic acid;
5-bromo-2-({4-[(methoxyacetyl)amino]benzoyl}amino)benzoic acid;
5-bromo-2-[(3-{[(6-chloropyridin-3-yl)carbonyl]amino}benzoyl)amino]benzoic acid;
5-bromo-2-[(4-{[(6-chloropyridin-3-yl)carbonyl]amino}benzoyl)amino]benzoic acid;
5-bromo-2-[(3-{[(3-chloro-1-benzothien-2-yl)carbonyl]amino}benzoyl)amino]benzoic acid;
5-bromo-2-[(4-{[(3-chloro-1-benzothien-2-yl)carbonyl]amino}benzoyl)amino]benzoic acid;
2-({4-[(anilinocarbonyl)amino]benzoyl}amino)-5-bromobenzoic acid;
5-bromo-2-{[4-({[(3-cyanophenyl)amino]carbonyl}amino)benzoyl]amino}benzoic acid;
5-bromo-2-{[4-({[(3,5-dichlorophenyl)amino]carbonyl}amino)benzoyl]amino}benzoic acid;
5-cyano-2-({3-(2,3-dihydro-1H-indol-1-ylsulfonyl)-4-[(hexanoylamino)methyl]benzoyl}amino)benzoic acid;
5-cyano-2-({3-(2,3-dihydro-1H-indol-1-ylsulfonyl)-4-[(undecanoylamino)methyl]benzoyl}amino)benzoic acid;
2-{[4-[(acetylamino)methyl]-3-(2,3-dihydro-1H-indol-1-ylsulfonyl)benzoyl]amino}-5-cyanobenzoic acid;
2-[(4-azido-3-iodobenzoyl)amino]-5-cyanobenzoic acid;
5-bromo-2-[(4-{[(6-methoxypyridin-3-yl)amino]sulfonyl}benzoyl)amino]benzoic acid;
5-bromo-2-({4-[(4-chloroanilino)sulfonyl]benzoyl}amino)benzoic acid;
5-bromo-2-[(4-{[(4-methoxyphenyl)(methyl)amino]sulfonyl}benzoyl)amino]benzoic acid;
2-[(4-fluorobenzoyl)amino]-5-iodobenzoic acid;
5-bromo-2-({4-[(6-chloro-5-fluoro-2,3-dihydro-1H-indol-1-yl)sulfonyl]benzoyl}amino)benzoic acid;
5-bromo-2-({4-[(1H-1,2,4-triazol-3-ylamino)sulfonyl]benzoyl}amino)benzoic acid;
5-bromo-2-{[4-(2,3-dihydro-1H-pyrrolo[2,3-b]pyridin-1-ylsulfonyl)benzoyl]amino}benzoic acid;
2-{[3-amino-4-(2,3-dihydro-1H-indol-1-ylsulfonyl)benzoyl]amino}-5-chlorobenzoic acid hydrochloride;
5-bromo-2-({4-[(2-hydroxyanilino)sulfonyl]benzoyl}amino)benzoic acid; and
5-bromo-2-{[4-(2,3-dihydro-1H-indol-1-ylcarbonyl)benzoyl]amino}benzoic acid.

15. A method of disinfecting or sanitizing comprising administering a therapeutically effective amount of a compound of claim 1.
